(19) United States
(12) Patent Application Publication
(10) Pub. No.: US 2025/0345431 A1
(43) Pub. Date: Nov. 13, 2025

(54) GENETICALLY ENGINEERED T CELLS EXPRESSING A CD19 CHIMERIC ANTIGEN RECEPTOR (CAR) AND USES THEREOF FOR ALLOGENEIC CELL THERAPY

(71) Applicant: Juno Therapeutics, Inc., Seattle, WA (US)

(72) Inventors: CALVIN CHAN, Seattle, WA (US); CEDRIC CLEYRAT, Seattle, WA (US); LUCREZIA COLONNA, Seattle, WA (US); CYR CLOVIS CHUA DE IMUS, Kenmore, WA (US); GABRIELA DIAZ, Seattle, WA (US); JENNA FERNANDEZ, Lynnwood, WA (US); NATALYA ALEKSANDRA GOLOVIZNINA, Seattle, WA (US); GABRIELA HERNANDEZ-HOYOS, Seattle, WA (US); CHRISTOPHER MARK HILL, Seattle, WA (US); XUEZHOU HOU, Shoreline, WA (US); AKSHATA IJANTKAR, Seattle, WA (US); DARREN M. KAMIKURA, Belle Mead, NJ (US); WEI-MING KAO, San Mateo, CA (US); ASHLEY KERN KOEGEL, San Francisco, CA (US); ALEXIS LITKE, Seattle, WA (US); RUTH AMANDA SALMON, Bainbridge Island, WA (US); JIANBIN WANG, Marysville, WA (US); PAUL RICHARD WEINGARDEN, Seattle, WA (US); JOHN-MICHAEL WILLIFORD, Seattle, WA (US); SARAH KATHERINE WILSON, Seattle, WA (US); PEI YANG, Newark, CA (US)

(73) Assignee: Juno Therapeutics, Inc., Seattle, WA (US)

(21) Appl. No.: 19/203,908

(22) Filed: May 9, 2025

Related U.S. Application Data
(60) Provisional application No. 63/645,442, filed on May 10, 2024.
(60) Provisional application No. 63/758,598, filed on Feb. 14, 2025.

(51) Int. Cl. A61K 40/42; A61K 40/11; A61K 40/31; A61K 40/34; A61K 40/50; A61P 35/00; C12N 5/0783; C12N 15/90

(52) U.S. Cl. CPC A61K 40/4211; A61K 40/11; A61K 40/31; A61K 40/34; A61K 40/50; A61P 35/00; C12N 5/0636; C12N 15/907; A61K 2239/48; C12N 2510/00

(57) ABSTRACT

Provided herein are genetically engineered T cells containing a chimeric antigen receptor (CARs), and related methods and uses thereof in allogeneic cell therapy. In some embodiments, the T cells are genetically engineered with a CAR and are further genetically engineered by one or more strategies to reduce host immune recognition of the engineered T cells, such as by heterologous expression of one or more additional transgenes and by genetic disruption to reduce or eliminate expression or one or more endogenous protein. Also provided are cell compositions containing the engineered T cells, and related methods, kits and systems for producing the engineered T cells. Also provided are methods of making and using the engineered T cells for cell therapy, including in connection with cancer immunotherapy comprising adoptive transfer of the engineered T cells.

### CROSS-REFERENCE TO RELATED APPLICATIONS

This application claims the benefit of U.S. Provisional Application No. 63/645,442, filed May 10, 2024, and U.S. Provisional Application No. 63/758,598, filed Feb. 14, 2025, which are incorporated by reference herein in their entirety for any purpose.

### SEQUENCE LISTING

The present application contains a Sequence Listing, which has been submitted electronically in XML format. Said XML file was created on May 1, 2025, is named “14682-WO-PCT_ST26.xml”, and is 201,270 bytes in size. The information in the electronic format of the sequence listing is incorporated herein by reference in its entirety.

### FIELD

The present disclosure relates in some aspects to genetically engineered cells such as T cells containing chimeric antigen receptors (CARs), and related methods and uses thereof in allogeneic cell therapy. In some embodiments, the T cells are genetically engineered with a CAR and are further genetically engineered by one or more strategies to reduce host immune recognition of the engineered T cells, such as by heterologous expression of one or more additional transgenes and by genetic disruption to reduce or eliminate expression or one or more endogenous protein. Also disclosed are cell compositions containing the engineered T cells, and related methods, kits and systems for producing the engineered T cells. Also provided are methods of making and using the engineered T cells for cell therapy, including in connection with cancer immunotherapy comprising adoptive transfer of the engineered T cells.

### BACKGROUND

Various cell therapy methods are available for treating diseases and conditions. Among cell therapy methods are methods involving immune cells, such as T cells, genetically engineered with a recombinant receptor, such as a chimeric antigen receptor (CAR). However, in some cases, current methods for generating CAR T cells are not ideal because they require patient-specific manufacturing for autologous delivery. Further, even for allogenic cell therapies, there is in many cases a problem with the persistence of the cell therapy in the subject so that there can be a high rate of relapse. Also, in some cases, incidences of relapse following CAR-T cell therapy may be high because of insufficient targeting of disease cells by the CAR due to antigen escape of the antigen being targeted by the CAR and/or heterogeneity in the character of tumor cells so that targeting a single antigen may be insufficient. Improved CAR T cell therapies are needed, including in connection with allogenic administration.

### SUMMARY

Provided herein is a genetically engineered T cell comprising: (a) a first genetic disruption in the endogenous TRAC gene; (b) a second genetic disruption in the endogenous B-2 microglobulin (B2M) gene; (c) a nucleotide sequence comprising a transgene encoding a single chain HLA-E fusion protein; and (d) a nucleotide sequence encoding a chimeric antigen receptor (CAR).

Also provided herein is a genetically engineered T cell comprising: (a) a first genetic disruption in the endogenous TRAC gene; (b) a second genetic disruption in the endogenous B-2 microglobulin (B2M) gene; (c) a nucleotide sequence encoding a single chain HLA-E fusion protein; and (d) a nucleotide sequence encoding a chimeric antigen receptor directed against CD19.

In some embodiments, the gene editing technique is or comprises a CRISPR-Cas system. In some embodiments, the Cas is a Cas9. In some embodiments, the Cas is a S. pyogenes Cas9 (spCas9). In some embodiments, the Cas is a Cas12a. In some embodiments, the Cas12 as is Francisella novicida Cas12a (FnCas12a), Lachnospiraceae bacterium Cas12a (LbCas12a), Acidaminococcus sp. Cas12a (AsCas12a).

In some of any embodiments, the first genetic disruption is by a CRISPR-Cas system that comprises a Cas protein and a guide RNA (gRNA) targeting the endogenous TRAC gene that comprises a spacer sequence that is complementary to a target site sequence in the endogenous TRAC gene, optionally wherein the Cas protein is a Cas9. In some of any embodiments, the first genetic disruption in the endogenous TRAC gene is in a target site sequence in exon 1 of the TRAC gene.

In some embodiments, the target site sequence in exon 1 of the endogenous TRAC gene is located within a TRAC genome region at contiguous positions within the hg38 genomic region chr14:22,547,506-22,547,778. In some of any embodiments, the target site sequence in exon 1 of the endogenous TRAC gene is located at hg38 genomic coordinates chr14:22,547,576-22,547,595. In some of any embodiments, the target site sequence in exon 1 of the endogenous TRAC gene has the sequence set forth in SEQ ID NO: 84, a contiguous portion thereof of at least 14 nucleotides (nt), or a complementary sequence of the foregoing. In some of any embodiments, the target site sequence in exon 1 of the endogenous TRAC gene has the sequence set forth in SEQ ID NO: 84.

In some of any embodiments, the first genetic disruption is by a CRISPR-Cas system that comprises a Cas9 protein and a guide RNA (gRNA) comprising a spacer sequence comprising the nucleic acid sequence of SEQ ID NO: 87, or a contiguous portion thereof of at least 14 nt. In some of any embodiments, the CRISPR-Cas system is a ribonucleoprotein (RNP) complex comprising the Cas9 protein and the gRNA.

In some of any embodiments, the first genetic disruption disrupts one or more alleles of the endogenous TRAC gene. In some of any embodiments, the first genetic disruption disrupts all alleles of the endogenous TRAC gene. In some of any embodiments, the first genetic disruption reduces protein expression of TCR alpha chain encoded from the endogenous TRAC gene, optionally protein expression of the TCR alpha chain on the surface of the T cell, more optionally wherein there is no detectable expression of TCR alpha chain in the T cell.

In some of any embodiments, the genetically engineered cell has reduced expression of CD3 on the cell surface, optionally wherein the genetically engineered cell does not express detectable CD3 on the cell surface.

In some of any embodiments, the second genetic disruption is by a CRISPR-Cas system that comprises a Cas protein and a guide RNA (gRNA) targeting the endogenous B2M gene that comprises a spacer sequence that is complementary to a target site sequence in the endogenous B2M gene, optionally wherein the Cas protein is a Cas12a. In some of any embodiments, the second genetic disruption in the endogenous B2M gene is in a target site sequence in exon 2 of the B2M gene.

In some embodiments, the target site sequence in exon 2 of the endogenous B2M gene is located within a B2M genome region at contiguous positions within hg38 the genomic region 44,715,423-44,715,701. In some of any embodiments, the target site sequence in exon 2 of the endogenous B2M gene is located at hg38 genomic coordinates chr15:44,715,614-44,715,634. In some of any embodiments, the target site sequence in exon 2 of the endogenous B2M gene has the sequence set forth in SEQ ID NO: 85, a contiguous portion thereof of at least 14 nucleotides (nt), or a complementary sequence of the foregoing. In some of any embodiments, the target site sequence has the sequence set forth in SEQ ID NO: 85.

In some of any embodiments, the second genetic disruption is by a CRISPR-Cas system that comprises a Cas12a protein and a guide RNA (gRNA) comprising a spacer sequence comprising the nucleic acid sequence SEQ ID NO:105, or a contiguous portion thereof of at least 14 nt. In some of any embodiments, the CRISPR-Cas system is a ribonucleoprotein (RNP) complex comprising the Cas12a protein and the gRNA.

In some of any embodiments, the second genetic disruption disrupts one or more alleles of the endogenous B2M gene. In some of any embodiments, the second genetic disruption disrupts all alleles of the endogenous B2M gene. In some of any embodiments, the second genetic disruption reduces protein expression of B2M encoded from the endogenous B2M gene, optionally wherein there is no detectable expression of endogenous B2M in the T cell.

In some of any embodiments, the genetically engineered cell has reduced expression of one or more HLA class I molecules (e.g., HLA-A class I, HLA-B class I and/or HLA-C class I) on the cell surface, optionally wherein the genetically engineered cell has no detectable expression of one or more HLA class I molecules (e.g., HLA-A class I, HLA-B class I and/or HLA-C class I) on the cell surface. In some of any embodiments, the genetically engineered cell has no detectable expression of HLA-A class I, HLA-B class I and HLA-C class I on the cell surface.

In some of any embodiments, each gRNA independently comprises a spacer sequence between 14 nt and 24 nt, or between 16 nt and 22 nt in length. In some of any embodiments, the gRNA independently comprises a spacer sequence that is 18 nt, 19 nt, 20 nt, 21 nt, or 22 nt in length. In some of any embodiments, each gRNA further comprises a scaffold sequence for binding the respective Cas protein. In some of any embodiments, the gRNA is modified by one or more modified nucleotides, wherein the one or more modified nucleotides are for increased stability of the gRNA.

In some of any embodiments, the gRNA targeting the endogenous TRAC gene comprises the sequence set forth in SEQ ID NO:82. In some of any embodiments, the gRNA targeting the endogenous B2M gene comprises the sequence set forth in SEQ ID NO:83.

In some of any embodiments, the nucleotide sequence encoding the single chain HLA-E fusion protein is present in the disrupted B2M gene in the T cell under the operable control of a promoter.

In some embodiments, the promoter is the endogenous promoter of the B2M gene. In some embodiments, the promoter is a heterologous promoter of the B2M gene.

In some of any embodiments, the nucleotide sequence has been integrated in the disrupted B2M gene by homology directed repair (HDR).

In some of any embodiments, the single chain HLA-E fusion protein comprises at least a portion of the B2M protein linked to at least a portion of an HLA-E class I chain. In some of any embodiments, the at least a portion of the B2M protein is linked to at least a portion of an HLA-E class I chain by a peptide linker. In some of any embodiments, the single chain HLA-E fusion protein further comprises a peptide linked to the fusion protein comprising at least a portion of the B2M and at least a portion of an HLA-E.

In some embodiments, the peptide is a peptide epitope that is presented by the single chain HLA-E fusion protein when expressed on the cell surface, optionally wherein presentation of the peptide on the cell surface ensures proper folding of the single chain fusion on the cell surface. In some of any embodiments, the peptide is a portion of a signal sequence from an MHC class I molecule. In some of any embodiments, the peptide is VMAPRTLVL (SEQ ID NO:107), VMAPRTLLL (SEQ ID NO:108), VMAPRTVLL (SEQ ID NO:109), VMAPRTLFL (SEQ ID NO: 110), or VMAPRTLIL (SEQ ID NO:111). In some of any embodiments, the peptide is VMAPRTLVL (SEQ ID NO:107).

In some of any embodiments, the peptide is linked to the fusion protein comprising at least a portion of the B2M protein and at least a portion of an HLA-E class I chain by a peptide linker. In some of any embodiments, the peptide linker is a GS linker, optionally wherein the GS linker is 4 to 25 amino acids in length, optionally wherein the GS linker is 12 to 20 amino acids in length, more optionally at or about 15 amino acids in length. In some embodiments, the GS linker is a (G4S)x3 linker is a

| (SEQ ID NO: 112) | |
|---|---|
| | GGGGSGGGGSGGGGS |

In some of any embodiments, the single chain HLA-E fusion protein comprises the sequence of amino acids set forth in SEQ ID NO:81 or a sequence of amino acids that has at least about 85%, 86%, 87%, 88%, 89%, 90%, 91%, 92%, 93%, 94%, 95%, 96%, 97%, 98%, or 99% sequence identity to SEQ ID NO:81. In some of any embodiments, the single chain HLA-E fusion protein comprises the sequence of amino acids set forth in SEQ ID NO:81. In some of any embodiments, the single chain fusion HLA-E fusion protein is capable of engaging inhibitory receptors on the surface of NK cells.

In some of any embodiments, the nucleotide sequence encoding the CAR is present in the disrupted TRAC gene in the T cell under the operable control of a promoter. In some embodiments, the promoter is a heterologous promoter of the TRAC gene. In some of any embodiments, the heterologous promoter is or comprises a human elongation factor 1 alpha (EF1α) promoter or a variant thereof. In some embodiments, the promoter is the endogenous promoter of the TRAC gene.

In some of any embodiments, the nucleotide sequence has been integrated in the disrupted TRAC gene by homology directed repair (HDR).

In some of any embodiments, (i) the VH region of the CD19-binding domain comprises the sequences set forth in, or a sequence that is at least 90%, 91%, 92%, 93%, 94%, 95%, 96%, 97%, 98% or 99% identical to, SEQ ID NO: 1; and (ii) the VL region of the CD19-binding domain comprises the sequences set forth in, or a sequence that is at least 90%, 91%, 92%, 93%, 94%, 95%, 96%, 97%, 98% or 99% identical to, SEQ ID NO: 2. In some of any embodiments, wherein the VH region of the CD19-binding domain comprises the sequences set forth in SEQ ID NO: 1; and the VL region of the CD19-binding domain comprises the sequences set forth in SEQ ID NO: 2.

In some of any embodiments, the VH region of the CD19-binding domain is joined to the VL region of the CD19-binding domain via a linker. In some embodiments, the linker is a flexible linker. In some of any embodiments, the linker is 5 to 25 amino acids in length, optionally wherein the linker is 12 to 18 amino acids in length. In some of any embodiments, the linker comprises the sequence set forth in SEQ ID NO: 18 or the sequence set forth in SEQ ID NO: 19.

In some of any embodiments, the length of the linker is between 5 and 25 amino acids, inclusive, optionally wherein the length of the linker is between 5 and 15 amino acids, inclusive. In some of any embodiments, the linker is a G4S linker (SEQ ID NO: 20), a G4S2 linker (SEQ ID NO: 21) or a (G4S)4 linker (SEQ ID NO: 22).

In some of any embodiments, the spacer comprises a hinge region sequence, optionally wherein the hinge region sequence is a hinge region of an immunoglobulin or a variant thereof. In some embodiments, the hinge region of an immunoglobulin is an IgG4 hinge region, optionally a human IgG4 hinge region, or a variant thereof. In some of any embodiments, the spacer comprises a variant IgG4 hinge region comprising substitution of amino acids CPSC to CPPC compared to the wild-type IgG4 hinge region. In some of any embodiments, the spacer is between 12 and 15 amino acids in length. In some of any embodiments, the spacer comprises an amino acid sequence having at least at or about 85%, 86%, 87%, 88%, 89%, 90%, 91%, 92%, 93%, 94%, 95%, 96%, 97%, 98% or 99% identity to SEQ ID NO: 12, optionally wherein the spacer has the sequence set forth in SEQ ID NO: 12. In some of any embodiments, the spacer is between 200 and 250 amino acids in length, or between 220 and 240 amino acids in length.

In some of any embodiments, the spacer comprises a hinge region of an immunoglobulin, a CH2 region of an immunoglobulin or a chimeric CH2 region of two different immunoglobulins, and a CH3 region of an immunoglobulin. In some of any embodiments, the spacer comprises an IgG4 hinge region or a variant thereof, a chimeric CH2 region comprising a portion of an IgG4 CH2 and a portion of an IgG2 CH2 (IgG2/4 CH2 region), and an IgG4 CH3 region. In some of any embodiments, the spacer comprises an amino acid sequence having at least at or about 85%, 86%, 87%, 88%, 89%, 90%, 91%, 92%, 93%, 94%, 95%, 96%, 97%, 98% or 99% identity to SEQ ID NO: 13, optionally wherein the spacer has the sequence set forth in SEQ ID NO: 13.

In some of any embodiments, the transmembrane domain comprises a transmembrane domain from CD28, optionally a human CD28. In some of any embodiments, the transmembrane domain is or comprises SEQ ID NO: 15 or an amino acid sequence having at least at or about 90%, 91%, 92%, 93%, 94%, 95%, 96%, 97%, 98% or 99% sequence identity to SEQ ID NO: 15.

In some of any embodiments, the intracellular signaling domain is a cytoplasmic signaling domain of a CD3-zeta (CD3ζ) chain, optionally a human CD3ζ chain. In some of any embodiments, the intracellular signaling domain comprises the sequence set forth in SEQ ID NO: 17, or an amino acid sequence having at least at or about 90%, 91%, 92%, 93%, 94%, 95%, 96%, 97%, 98% or 99% sequence identity to SEQ ID NO: 17.

In some of any embodiments, the intracellular signaling region further comprises a costimulatory signaling region. In some embodiments, the costimulatory signaling region comprises an intracellular signaling domain of a T cell costimulatory molecule or a signaling portion thereof. In some of any embodiments, wherein the costimulatory signaling region comprises an intracellular signaling domain of 4-1BB, optionally a human 4-1BB. In some of any embodiments, the costimulatory signaling region comprises the sequence set forth in SEQ ID NO: 16 or an amino acid sequence having at least at or about 90%, 91%, 92%, 93%, 94%, 95%, 96%, 97%, 98% or 99% sequence identity to SEQ ID NO: 16.

In some of any embodiments, the CAR comprises the amino acid sequence set forth in SEQ ID NO: 78 or SEQ ID NO: 138, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 78 or SEQ ID NO: 138.

In some of any embodiments, the genetically engineered T cell comprises one or more further genetic disruptions to reduce cell surface expression of one or more HLA class II molecules. In some embodiments, the one or more further genetic disruptions is a genetic disruption in the CIITA gene.

In some of any embodiments, the T cell is a primary T cell. In some embodiments, the primary T cell is from a human donor. In some embodiments, the human donor is a healthy donor.

Also provided herein is a method of producing a genetically engineered T cell, the method comprising: (a) introducing, into a T cell, a first agent for inducing a first genetic disruption at a target site sequence in an endogenous endogenous B-2 microglobulin (B2M) gene; (b) introducing into the T cell a second agent for inducing a second genetic disruption at a target site sequence in a endogenous T cell receptor alpha constant (TRAC) gene; (c) introducing into the T cell a polynucleotide comprising a transgene encoding a single chain HLA-E fusion protein; and (d) introducing into the T cell a polynucleotide comprising a transgene encoding a chimeric antigen receptor (CAR).

Also provided herein is a method of producing a genetically engineered T cell, the method comprising: (a) introducing, into a T cell, a first agent for inducing a first genetic disruption at a target site sequence in an endogenous endogenous B-2 microglobulin (B2M) gene; (b) introducing into the T cell a second agent for inducing a second genetic disruption at a target site sequence in a endogenous T cell receptor alpha constant (TRAC) gene; (c) introducing into the T cell a polynucleotide comprising a transgene encoding a single chain HLA-E fusion protein; and (d) introducing into the T cell a polynucleotide comprising a transgene encoding a chimeric antigen receptor (CAR) directed against CD19.

In some of any embodiments, each genetic disruption is by a gene editing technique. In some embodiments, each introduced agent mediates the gene editing technique and is or comprises a CRISPR-Cas system comprising a guide RNA (gRNA) comprising a spacer sequence that binds to the target site and a Cas protein. In some embodiments, each CRISPR-Cas system is a ribonucleoprotein (RNP) complex comprising the Cas protein and the gRNA. In some of any embodiments, the first agent is a first CRISPR-Cas system comprising a guide RNA (gRNA) targeting the endogenous TRAC gene comprising a spacer sequence that is complementary to the target site in the endogenous TRAC gene, and a Cas9 protein. In some embodiments, the CRISPR-Cas system is a ribonucleoprotein (RNP) complex comprising the Cas9 protein and the gRNA. In some of any embodiments, the Cas is a S. pyogenes Cas9 (spCas9).

In some of any embodiments, the target site sequence in the endogenous T cell receptor alpha constant (TRAC) gene is in exon 1 of the TRAC gene. In some of any embodiments, the target site sequence in the endogenous TRAC gene is located within a TRAC genome region at contiguous positions within the hg38 genomic region chr14:22,547,506-22,547,778. In some of any embodiments, the target site sequence in the endogenous TRAC gene is located at hg38 genomic coordinates chr14:22,547,576-22,547,595. In some of any embodiments, the target site sequence in the endogenous TRAC gene has the sequence set forth in SEQ ID NO: 84, a contiguous portion thereof of at least 14 nucleotides (nt), or a complementary sequence of the foregoing. In some of any embodiments, the target site sequence in the endogenous TRAC gene has the sequence set forth in SEQ ID NO: 84.

In some of any embodiments, the gRNA comprises a spacer sequence comprising the nucleic acid sequence of SEQ ID NO: 87, or a contiguous portion thereof of at least 14 nt.

In some of any embodiments, the first genetic disruption disrupts one or more alleles of the endogenous TRAC gene. In some of any embodiments, the first genetic disruption disrupts all alleles of the endogenous TRAC gene.

In some of any embodiments, introducing the first agent into the T cell reduces protein expression of TCR alpha chain encoded from the endogenous TRAC gene, optionally protein expression of the TCR alpha chain on the surface of the T cell, more optionally wherein there is no detectable expression of TCR alpha chain in the T cell. In some of any embodiments, introducing the first agent into the T cell reduces expression of CD3 on the cell surface, optionally where there is no detectable CD3 on the cell surface.

In some of any embodiments, the second agent is a second CRISPR-Cas system comprising a guide RNA (gRNA) targeting the endogenous B2M gene comprising a spacer sequence that is complementary to the target site in the endogenous B2M gene, and a Cas12a protein. In some embodiments, the CRISPR-Cas system is a ribonucleoprotein (RNP) complex comprising the Cas12a protein and the gRNA. In some of any embodiments, wherein the Cas is a Cas12a is Francisella novicida Cas12a (FnCas12a), Lachnospiraceae bacterium Cas12a (LbCas12a), Acidaminococcus sp. Cas12a (AsCas12a).

In some of any embodiments, the target site sequence in the endogenous B2M gene is in exon 2 of the B2M gene. In some of any embodiments, the target site sequence in the endogenous B2M gene is located within a B2M genome region at contiguous positions within hg38 the genomic region 44,715,423-44,715,701. In some of any embodiments, the target site sequence in the endogenous B2M gene is located at hg38 genomic coordinates chr15:44,715,614-44,715,634. In some of any embodiments, the target site sequence in the endogenous B2M gene has the sequence set forth in SEQ ID NO: 85, a contiguous portion thereof of at least 14 nucleotides (nt), or a complementary sequence of the foregoing. In some of any embodiments, the target site sequence in the endogenous B2M gene has the sequence set forth in SEQ ID NO: 85.

In some of any embodiments, the gRNA comprises a spacer sequence comprising the nucleic acid sequence of SEQ ID NO: 105, or a contiguous portion thereof of at least 14 nt.

In some of any embodiments, wherein the second genetic disruption disrupts one or more alleles of the endogenous B2M gene. In some of any embodiments, the second genetic disruption disrupts all alleles of the endogenous B2M gene.

In some of any embodiments, introducing the second agent into the T cell reduces protein expression of B2M encoded from the endogenous B2M gene, optionally wherein there is no detectable expression of B2M in the T cell.

In some of any embodiments, introducing the second agent into the T cell reduces expression of one or more HLA class I molecules (e.g., HLA-A class I, HLA-B class I and/or HLA-C class I) on the cell surface, optionally wherein there is no detectable expression of one or more HLA class I molecules (e.g., HLA-A class I, HLA-B class I and/or HLA-C class I) on the cell surface. In some of any embodiments, introducing the second agent into the T cell results in no detectable expression of HLA-A class I, HLA-B class I and HLA-C class I on the cell surface.

In some of any embodiments, wherein each gRNA independently comprises a spacer sequence between 14 nt and 24 nt, or between 16 nt and 22 nt in length. In some of any embodiments, wherein each gRNA independently comprises a spacer sequence that is 18 nt, 19 nt, 20 nt, 21 nt, or 22 nt in length. In some of any embodiments, each gRNA further comprises a scaffold sequence for binding the respective Cas protein. In some of any embodiments, the gRNA is modified by one or more modified nucleotides, wherein the one or more modified nucleotides are for increased stability of the gRNA.

In some of any embodiments, the gRNA targeting the endogenous TRAC gene comprises the sequence set forth in SEQ ID NO: 82 or SEQ ID NO: 92. In some of any embodiments, the gRNA targeting the endogenous B2M gene comprises the sequence set forth in SEQ ID NO: 83. In some of any embodiments, the gRNA targeting the endogenous TRAC gene and/or the gRNA targeting the endogenous B2M gene induces a double strand break.

In some of any embodiments, the transgene encoding a single chain HLA-E fusion protein is integrated via homology directed repair (HDR) at the target site in the B2M gene.

In some of any embodiments, the polynucleotide encoding the single chain HLA-E fusion protein further comprises one or more homology arm(s) linked to the transgene, wherein the one or more homology arm(s) comprise a sequence homologous to nucleic acid sequences surrounding the target site sequence in the endogenous B2M gene. In some embodiments, the polynucleotide encoding the single chain HLA-E fusion protein comprises the structure [5′ homology arm]-[transgene]-[3′ homology arm], wherein the 5′ homology arm and 3′ homology arm comprises nucleic acid sequences homologous to the nucleic acid sequences surrounding the target site sequence in the endogenous B2M gene.

In some of any embodiments, the 5′ homology arm and 3′ homology arm independently are at or about 200, 300, 400, 500, 600, 700 or 800 nucleotides in length, or any value between any of the foregoing. In some of any embodiments, the 5′ homology arm comprises SEQ ID NO: 79 or a sequence that has at least at or about 90%, 91%, 92%, 93%, 94%, 95%, 96%, 97%, 98% or 99% sequence identity to SEQ ID NO: 79 or a partial sequence thereof, and/or the 3′ homology arm comprises SEQ ID NO: 80, a sequence that has at least at or about 90%, 91%, 92%, 93%, 94%, 95%, 96%, 97%, 98% or 99% sequence identity to SEQ ID NO: 80 or a partial sequence thereof. In some of any embodiments, the 5′ homology arm comprises SEQ ID NO: 79 and the 3′ homology arm comprises SEQ ID NO: 80.

In some of any embodiments, the single chain HLA-E fusion protein comprises at least a portion of the B2M protein linked to at least a portion of an HLA-E class I chain, optionally via a peptide linker. In some embodiments, the single chain HLA-E fusion comprises a peptide sequence, wherein the peptide is a peptide epitope that is presented by the single chain HLA-E fusion protein when expressed on the cell surface. In some embodiments, the peptide is VMAPRTLVL (SEQ ID NO: 107), VMAPRTLLL (SEQ ID NO: 108), VMAPRTVLL (SEQ ID NO: 109), VMAPRTLFL (SEQ ID NO: 110), or VMAPRTLIL (SEQ ID NO: 111), optionally wherein the peptide is VMAPRTLVL (SEQ ID NO: 107).

In some of any embodiments, the single chain HLA-E fusion protein comprises the sequence of amino acids set forth in SEQ ID NO: 81 or a sequence of amino acids that has at least about 85%, 86%, 87%, 88%, 89%, 90%, 91%, 92%, 93%, 94%, 95%, 96%, 97%, 98%, or 99% sequence identity to SEQ ID NO: 81.

In some of any embodiments, the transgene encoding the CAR is integrated via homology directed repair (HDR) at the target site in the TRAC gene.

In some of any embodiments, the polynucleotide encoding the CAR further comprises one or more homology arm(s) linked to the transgene, wherein the one or more homology arm(s) comprise a sequence homologous to nucleic acid sequences surrounding the target site sequence in the endogenous TRAC gene. In some embodiments, the polynucleotide encoding the CAR comprises the structure [5′ homology arm]-[transgene]-[3′ homology arm], wherein the 5′ homology arm and 3′ homology arm comprises nucleic acid sequences homologous to the nucleic acid sequences surrounding the target site sequence in the endogenous TRAC gene.

In some of any embodiments, the 5′ homology arm and 3′ homology arm independently are at or about 200, 300, 400, 500, 600, 700 or 800 nucleotides in length, or any value between any of the foregoing. In some of any embodiments, the 5′ homology arm comprises SEQ ID NO: 76 or a sequence that has at least at or about 90%, 91%, 92%, 93%, 94%, 95%, 96%, 97%, 98% or 99% sequence identity to SEQ ID NO: 76 or a partial sequence thereof, and/or the 3′ homology arm comprises SEQ ID NO: 77, a sequence that has at least at or about 90%, 91%, 92%, 93%, 94%, 95%, 96%, 97%, 98% or 99% sequence identity to SEQ ID NO: 77 or a partial sequence thereof. In some of any embodiments, the 5′ homology arm comprises SEQ ID NO: 76 and the 3′ homology arm comprises SEQ ID NO: 77.

In some of any embodiments, the CAR is directed against CD19. In some of any embodiments, the CAR comprises an extracellular domain, a spacer, a transmembrane domain, and an intracellular signaling domain. In some embodiments, the extracellular domain comprises a CD19-binding domain that binds to CD19 comprising a VH region and a VL region.

In some of any embodiments, (i) the VH region of the CD19-binding domain comprises the sequences set forth in, or a sequence that is at least 90%, 91%, 92%, 93%, 94%, 95%, 96%, 97%, 98% or 99% identical to, SEQ ID NO: 1; and (ii) the VL region of the CD19-binding domain comprises the sequences set forth in, or a sequence that is at least 90%, 91%, 92%, 93%, 94%, 95%, 96%, 97%, 98% or 99% identical to, SEQ ID NO: 2. In some of any embodiments, the VH region of the CD19-binding domain comprises the sequences set forth in SEQ ID NO: 1; and the VL region of the CD19-binding domain comprises the sequences set forth in SEQ ID NO: 2.

In some of any embodiments, the spacer comprises a hinge region sequence, optionally wherein the hinge region sequence is a hinge region of an immunoglobulin or a variant thereof.

In some of any embodiments, the transmembrane domain comprises a transmembrane domain from CD28, optionally a human CD28.

In some of any embodiments, the intracellular signaling domain is a cytoplasmic signaling domain of a CD3-zeta (CD3ζ) chain, optionally a human CD3ζ chain. In some of any embodiments, the intracellular signaling region further comprises a costimulatory signaling region comprising an intracellular signaling domain of a T cell costimulatory molecule or a signaling portion thereof. In some of any embodiments, the costimulatory signaling region comprises an intracellular signaling domain of 4-1BB, optionally a human 4-1BB.

In some of any embodiments, the transgene encoding the single chain HLA-E fusion is integrated to be under the operable control of the endogenous B2M promoter, optionally wherein the transgene encoding the single chain HLA-E fusion protein comprises one or more multicistronic element(s) positioned upstream of the nucleotide sequence encoding the single chain HLA-E fusion, more optionally wherein the one or more multicistronic element is or comprises a T2A, a P2A, an E2A, or an F2A element.

In some of any embodiments, the transgene encoding the CAR is operably linked to a heterologous promoter to control expression of the CAR. In some embodiments, the heterologous promoter is or comprises a human elongation factor 1 alpha (EF1α) promoter or a variant thereof.

In some of any embodiments, the introducing of the polynucleotide comprising a transgene encoding the single chain HLA-E fusion protein is by transduction of a first viral vector comprising the polynucleotide encoding the single chain HLA-E fusion; and/or the introducing of the polynucleotide comprising a transgene encoding the CAR is by transduction of a second viral vector comprising the polynucleotide comprising a transgene encoding the CAR. In some embodiments, a mixture comprising a first viral vector and a second viral vector are introduced into the T cell. In some of any embodiments, the first viral vector and the second viral vector is an AAV vector, optionally wherein the AAV vector is an AAV6 vector.

Also provided herein is a system for engineering a T cell, comprising: (a) a first agent for inducing a first genetic disruption at a target site sequence in an endogenous B-2 microglobulin (B2M) gene; (b) a second agent for inducing a second genetic disruption at a target site sequence in a endogenous T cell receptor alpha constant (TRAC) gene; (c) a polynucleotide comprising a transgene encoding a single chain HLA-E fusion protein; and (d) a polynucleotide comprising a transgene encoding a chimeric antigen receptor (CAR).

Also provided herein is a system for engineering a T cell, comprising: (a) a first agent for inducing a first genetic disruption at a target site sequence in an endogenous B-2 microglobulin (B2M) gene; (b) a second agent for inducing a second genetic disruption at a target site sequence in a endogenous T cell receptor alpha constant (TRAC) gene; (c) a polynucleotide comprising a transgene encoding a single chain HLA-E fusion protein; and (d) a polynucleotide comprising a transgene encoding a chimeric antigen receptor (CAR) directed against CD19.

In some of any embodiments, the first agent and/or second agent is or comprises a CRISPR-Cas system comprising a guide RNA (gRNA) comprising a spacer sequence that binds to the target site and a Cas protein. In some embodiments, each CRISPR-Cas system is a ribonucleoprotein (RNP) complex comprising the Cas protein and the gRNA. In some of any embodiments, the first agent is a ribonucleoprotein complex comprising a guide RNA (gRNA) targeting the endogenous TRAC gene comprising a spacer sequence that is complementary to the target site in the endogenous TRAC gene, and a Cas9 protein, optionally wherein the Cas is a S. pyogenes Cas9 (spCas9).

In some of any embodiments, the target site sequence in the endogenous T cell receptor alpha constant (TRAC) gene is in exon 1 of the TRAC gene. In some of any embodiments, the target site sequence in the endogenous TRAC gene has the sequence set forth in SEQ ID NO: 84, a contiguous portion thereof of at least 14 nucleotides (nt), or a complementary sequence of the foregoing.

In some of any embodiments, the gRNA comprises a spacer sequence comprising the nucleic acid sequence of SEQ ID NO: 87, or a contiguous portion thereof of at least 14 nt.

In some of any embodiments, the second agent is a ribonucleoprotein complex comprising a guide RNA (gRNA) targeting the endogenous B2M gene comprising a spacer sequence that is complementary to the target site in the endogenous B2M gene, and a Cas12a protein.

In some of any embodiments, the target site sequence in the endogenous B2M gene is in exon 2 of the B2M gene. In some of any embodiments, the target site sequence in the endogenous B2M gene has the sequence set forth in SEQ ID NO: 85, a contiguous portion thereof of at least 14 nucleotides (nt), or a complementary sequence of the foregoing.

In some of any embodiments, the gRNA comprises a spacer sequence comprising the nucleic acid sequence of SEQ ID NO: 105, or a contiguous portion thereof of at least 14 nt.

In some of any embodiments, the gRNA targeting the endogenous TRAC gene comprises the sequence set forth in SEQ ID NO:135. In some of any embodiments, the gRNA targeting the endogenous B2M gene comprises the sequence set forth in SEQ ID NO:136.

In some of any embodiments, the polynucleotide encoding the single chain HLA-E fusion protein further comprises one or more homology arm(s) linked to the transgene, wherein the one or more homology arm(s) comprise a 5′ homology arm and a 3′ homology arm comprises nucleic acid sequences homologous to the nucleic acid sequences surrounding the target site sequence in the endogenous B2M gene. In some embodiments, the 5′ homology arm comprises SEQ ID NO: 79 or a sequence that has at least at or about 90%, 91%, 92%, 93%, 94%, 95%, 96%, 97%, 98% or 99% sequence identity to SEQ ID NO: 79 or a partial sequence thereof, and/or the 3′ homology arm comprises SEQ ID NO: 80, a sequence that has at least at or about 90%, 91%, 92%, 93%, 94%, 95%, 96%, 97%, 98% or 99% sequence identity to SEQ ID NO: 80 or a partial sequence thereof.

In some of any embodiments, the single chain HLA-E fusion protein comprises the sequence of amino acids set forth in SEQ ID NO: 81 or a sequence of amino acids that has at least about 85%, 86%, 87%, 88%, 89%, 90%, 91%, 92%, 93%, 94%, 95%, 96%, 97%, 98%, or 99% sequence identity to SEQ ID NO: 81.

In some of any embodiments, the polynucleotide encoding the CAR further comprises one or more homology arm(s) linked to the transgene, wherein the one or more homology arm(s) comprise a 5′ homology arm and a 3′ homology arm comprising nucleic acid sequences homologous to the nucleic acid sequences surrounding the target site sequence in the endogenous TRAC gene. In some embodiments, the 5′ homology arm comprises SEQ ID NO: 76 or a sequence that has at least at or about 90%, 91%, 92%, 93%, 94%, 95%, 96%, 97%, 98% or 99% sequence identity to SEQ ID NO: 76 or a partial sequence thereof, and/or the 3′ homology arm comprises SEQ ID NO: 77, a sequence that has at least at or about 90%, 91%, 92%, 93%, 94%, 95%, 96%, 97%, 98% or 99% sequence identity to SEQ ID NO: 77 or a partial sequence thereof.

In some of any embodiments, (i) the VH region of the CD19-binding domain comprises the sequences set forth in, or a sequence that is at least 90%, 91%, 92%, 93%, 94%, 95%, 96%, 97%, 98% or 99% identical to, SEQ ID NO: 1; and (ii) the VL region of the CD19-binding domain comprises the sequences set forth in, or a sequence that is at least 90%, 91%, 92%, 93%, 94%, 95%, 96%, 97%, 98% or 99% identical to, SEQ ID NO: 2.

In some of any embodiments, the polynucleotide comprising a transgene encoding the single chain HLA-E fusion protein is comprised in a first viral vector comprising the polynucleotide encoding the single chain HLA-E fusion; and/or the polynucleotide comprising a transgene encoding the CAR is comprised in a second viral vector comprising the polynucleotide comprising a transgene encoding the CAR.

In some embodiments, the system comprises a mixture comprising a first viral vector and a second viral vector. In some of any embodiments, wherein the first viral vector and the second viral vector is an AAV vector, optionally wherein the AAV vector is an AAV6 vector.

Also provided herein is a kit comprising any of the systems disclosed herein, and optionally instructions for using the system to genetically engineer a T cell.

Also provided herein is a method of producing a genetically engineered T cell, the method comprising introducing the first agent, the second agent, the third agent, the polynucleotide comprising a transgene encoding an HLA-E fusion protein and the polynucleotide encoding the CAR of any of the systems disclosed herein into a T cell.

Also provided herein is a method of producing a genetically engineered T cell, the method comprising introducing the first agent, the second agent, the polynucleotide comprising a transgene encoding an HLA-E fusion protein and the polynucleotide encoding the CAR of any of the systems disclosed here into a T cell.

In some of any embodiments, the first agent and the second agent are introduced into the T cell via electroporation. In some embodiments, each of the first agent and second agent are independently introduced as a ribonucleoprotein complex (RNP) and the total concentration of the RNPs introduced into the T cell is between at or about 1 μM and at or about 5 μM, between at or about 1.5 μM and at or about 2.5 μM, between at or about 1.7 μM and at or about 2.5 μM, or between at or about 2 μM and at or about 2.5 μM, optionally at or about 1.0 μM, at or about 1.5 μM, at or about 1.7 μM, at or about 2 μM, at or about 2.2 μM, or at or about 2.5 μM.

In some of any embodiments, after the electroporation, the method comprises introducing the polynucleotides by transducing the T cells with a mixture of viral vectors, wherein the mixture of viral vectors comprises a first viral vector comprising the polynucleotide encoding the single chain HLA-E fusion and a second viral vector comprising the polynucleotide comprising a transgene encoding the CAR. In some embodiments, the first viral vector and the second viral vector is an AAV vector, optionally wherein the AAV vector is an AAV6 vector. In some of any embodiments, transducing is within about 15 minutes, within about 30 minutes, within about 60 minutes, or within about 2 hours, after the introductions.

In some of any embodiments, after the transducing the method further comprises incubating the cell under static conditions in serum free media for a period of time for recovery of the cells. In some of any embodiments, the method further comprises incubating the cells with one or more recombinant cytokines under conditions for expansion of T cells, optionally one or more recombinant IL-2, IL-7 and/or IL-15, optionally wherein expansion is carried out for 2 to 8 doublings of the T cells. In some embodiments, the incubating is carried out with perfusion.

In some of any embodiments, prior to each of the introducing, the method comprises stimulating the T cells with one or more stimulatory agent(s) under conditions to stimulate or activate the T cells, optionally wherein the one or more stimulatory agent(s) comprises anti-CD3 and/or anti-CD28 antibodies, optionally anti-CD3/anti-CD28 Fabs. In some of any embodiments, the T cell is a primary T cell. In some embodiments, the primary T cell is from a human donor. In some embodiments, the human donor is a healthy donor.

In some of any embodiments, the method disclosed herein is performed ex vivo. In some of any embodiments, the method is performed in vitro. In some of any embodiments, the method disclosed herein comprises harvesting T cells produced by the method.

In some embodiments, the method disclosed herein further comprises depleting CD3+ T cells from the harvested T cells. In some of any embodiments, the method disclosed herein further comprises formulating the harvested T cells with a cryoprotectant.

Also provided herein is composition comprising a population of genetically engineered T cells disclosed herein. Also provided herein is a composition comprising a population of genetically engineered T cells produced by any of the methods disclosed herein. In some of any embodiments, wherein the composition is a pharmaceutical composition comprising a pharmaceutically acceptable excipient. In some of any embodiments, the composition comprises a cyroprotectant, optionally wherein the cryoprotectant is DMSO.

In some of any embodiments, at least at or about 70%, 75%, 80%, 85%, 90%, or 95% of the engineered T cells, or of the total cells or total T cells, in the composition comprise a genetic disruption in the endogenous TRAC gene. In some of any embodiments, at least at or about 70%, 75%, 80%, 85%, 90%, or 95% of the engineered T cells, or of the total cells or total T cells, in the composition comprise no detectable expression of TCR alpha chain in the T cell. In some of any embodiments, at least at or about 70%, 75%, 80%, 85%, 90%, or 95% of the engineered T cells, or of the total cells or total T cells, in the composition comprise no detectable expression of CD3 on the cell surface of the T cell. In some of any embodiments, at least at or about 95%, 96%, 97% or 98% of the engineered T cells, or of the total cells or total T cells, in the composition comprise no detectable expression of CD3 on the cell surface of the T cell.

In some of any embodiments, at least at or about 70%, 75%, 80%, 85%, 90%, or 95% of the engineered T cells, or of the total cells or total T cells, in the composition comprise a genetic disruption in the endogenous B2M gene. In some of any embodiments, at least at or about 70%, 75%, 80%, 85%, 90%, or 95% of the engineered T cells, or of the total cells or total T cells, in the composition comprise no detectable expression of B2M in the T cell.

In some of any embodiments, at least at or about 70%, 75%, 80%, 85%, 90%, or 95% of the engineered T cells, or of the total cells or total T cells, in the composition comprise no detectable expression of one or more HLA class I molecules (e.g., HLA-A class I, HLA-B class I and/or HLA-C class I) on the cell surface. In some of any embodiments, at least at or about 70%, 75%, 80%, 85%, 90%, or 95% of the engineered T cells, or of the total cells or total T cells, in the composition comprise no detectable expression of HLA-A class I, HLA-B class I and HLA-C class I on the cell surface.

In some of any embodiments, at least at or about 50%, 60%, 65%, 70%, 75%, 80%, 85%, 90%, or 95% of the engineered T cells, or of the total cells or total T cells, in the composition express the single chain HLA-E fusion. In some of any embodiments, at least at or about 50%, 60%, 65%, 70%, 75%, 80%, 85%, 90%, or 95% of the engineered T cells, or of the total cells or total T cells, in the composition express the CAR. In some of any embodiments, at least at or about 50%, 60%, 65%, 70%, 75%, 80%, 85%, 90%, or 95% of the engineered T cells, or of the total cells or total T cells, in the composition express the single chain HLA-E fusion and the CAR.

In some of any embodiments, at least 90% of the total cells in the composition comprise a genetic disruption in the endogenous TRAC gene, at least 90% of the total cells in the composition comprise a genetic disruption in the endogenous B2M gene, at least 50% of the total cells in the composition express the HLA-E fusion and at least 50% of the total cells in the composition express the CAR.

In some of any embodiments, at least at or about 95%, 96%, 97% or 98% of the total cells in the composition comprise no detectable expression of CD3 on the cell surface of the T cell. In some of any embodiments, at least at or about 95%, 96%, 97% or 98% of the total cells in the composition comprise no detectable expression of HLA-A class I, HLA-B class I and HLA-C class I on the cell surface.

In some of any embodiments, at least at or about 90%, 91%, 92%, 93%, 94%, 95%, 96%, 97%, 98%, or 99% of the cells in the composition are T cells. In some of any embodiments, the composition comprises CD4+ T cells and CD8+ T cells. In some embodiments, the ratio of CD4+ T cells to CD8+ T cells is from at or about 1:5 to at or about 5:1, optionally from at or about 1:3 to at or about 3:1.

In some of any embodiments, at least at or about 80%, 85%, 90%, or 95% of the engineered T cells, or of the total cells or total T cells, in the composition are viable cells. In some of any embodiments, at least at or about 90%, 91%, 92%, 93%, 94%, 95%, 96%, 97%, 98%, or 99% of the cells in the composition are engineered T cells comprising a genetic disruption of one or more endogenous genes and expression of one or more transgene.

Also provided herein is a method of treatment, the method comprising administering any of the cells disclosed herein or the composition of any one of the compositions disclosed herein to a subject having a disease or disorder. In some embodiments, the disease or disorder is associated with an antigen targeted by the CAR. In some embodiments, the antigen is CD19.

In some of any embodiments, the disease or disorder is a cancer. In some of any embodiments, the disease or disorder is an autoimmune disease. In some embodiments, the cancer is a lymphoma or a leukemia. In some embodiments, the cancer is a lymphoma that is a large B cell lymphoma. In some of any embodiments, the lymphoma is a non-Hodgkin lymphoma.

### BRIEF DESCRIPTION OF THE DRAWINGS

FIG. 1 depicts the percent CD4+CCR7+CD45RA+ of live CD3+ T cells (y-axis) against healthy donor age in years (x-axis) (FIG. 1A), and the percent CD8+CCR7+CD45RA+ of live CD3+ T cells (y-axis) against healthy donor age in years (x-axis) (FIG. 1B).

FIG. 2 is a graph plotting the percent CD3+ T cells against healthy donor body mass index (BMI) (x-axis).

FIG. 3 depicts a schematic of the gene-edited T cell product (HD Allo CD19 CAR-T). The TRAC and B2M loci are disrupted, thus preventing endogenous TCR and HLA-I expression, respectively. An HLA-E fusion protein and CD19 CAR transgenes are delivered using rAAV6 vector for site-specific integration at the B2M and TRAC loci, respectively.

FIGS. 4 A, B, and C depict in vitro characterization of HD Allo CD19 CAR-T cells from three healthy donors in a Raji Burkitt's lymphoma 3-D spheroid tumor assay. FIG. 4A shows total integrated fluorescence intensity measured in co-cultures (effector to target cell ratio of 1:2) by monitoring NucLight Red fluorescence. FIG. 4B shows target-cell lysis for each donor by plotting total integrated intensity and the calculating the area under the curve (AUC). FIG. 4C shows measurement of proinflammatory cytokines in supernatants collected 24 hours post co-culture with HD Allo CD19 CAR-T from three donors and 3-D spheroid tumors using an electrochemiluminescence cytokine immunoassay.

FIGS. 5 A, B, and C depict in vitro cytotoxic activity of HD Allo CD19 CAR-T cells against B Cell Targets. B cells isolated from healthy and SLE donor material (3 HD; iSLE) were fluorescently labeled with Carboxyfluorescein Diacetate Succinimidyl Ester (CSFE) then co-cultured with HD Allo CD19 CAR-T cells in vitro at an effector to target cell ratio of 0.125:1 (12.5K effectors and 100K targets) and 2:1 (200K effectors and 100K targets) for 72 hours. Target B cell counts (FIG. 5A) and fold-expansion (FIG. 5B) were measured by flow cytometry and quantified using CountBright Absolute Counting Beads. Target B cell counts were gated on CSFE+/Caspase3− cells and fold expansion of HD Allo CD19 CAR-T cells were gated on CFSE−/CAR+ cells. Fold expansion of HD Allo CD19 CAR-T cells was calculated by dividing the HD Allo CD19 CAR-T cell counts at 72 hours by HD Allo CD19 CAR-T cell counts at 0 hours. HD Allo CD19 CAR-T cell activation and degranulation (FIG. 5C) were measured by quantification of CD38+CAR+ gMFI and CD107a+CAR+gMFI, respectively.

FIGS. 6 A and B depict a strategy to mitigate the effects of GvHD by knocking out the TRAC gene in the Allo CAR-T cells, which results in the abrogation of the expression of the endogenous TCR in Allo CAR T cells. To determine GvH alloreactivity, 1.0×10⁶ CSFE-labeled, unedited mock T cells or Allo CAR T cells derived from 3 healthy donors were incubated with HLA-mismatched host dendritic cells from 3 donors in vitro for 6 days and alloreactivity was determined by CSFE dilution.

FIG. 6A is a FACS plot from one of the 9 donor pairs. FIG. 6B is a graph showing combined data from the 9 donor pairs.

FIG. 7 depicts the protection of HD Allo CD19 CAR-T cells from NK-mediated CAR-T Cell Depletion in vitro. HD Allo CD19 CAR-T cells and allo CD19 B2M KO/No HLA-E KI CAR T cells were generated from 3 healthy donors and were plated at an E:T ratio of 0.6:1 with primary NK cells from 2 donors for 72 hours. After co-culture for 72 hours, cells were analyzed by multicolor flow cytometry to identify the percentage of live CD5+/CD56−/CAR+ T cells. (FlowJo software, TreeStar Inc., Ashland, OR). Data are displayed as group means±SEM. Data were analyzed using GraphPad Prism software (GraphPad Software, La Jolla, CA).

FIG. 8 depicts the in vivo activity of HD Allo CD19 CAR-T cells in Raji xenograft model. A single dose (1.0×10⁶ or 3.0×10⁶) of HD Allo CD19 CAR-T cells was injected intravenously into Raji Burkitt's lymphoma xenograft mice. Tumor growth and CAR T cell expansion were assessed.

### DETAILED DESCRIPTION

Provided herein are T cells engineered with a chimeric antigen receptor (CAR) that are further genetically engineered to have reduced recognition by the host immune response. In some embodiments, provided T cells are genetically engineered with a CAR and are genetically engineered by one or more strategies to mitigate graft versus host and host versus graft interaction as well as NK cell-mediated rejection, while preserving and in some cases enhancing T cell functions. In some embodiments, the provided engineered CAR T cell therapies are non-alloreactive so that they are not susceptible to, or exhibit reduced susceptibility compared to T cells without the genetic disruptions or modifications, to host immune system rejection. Also provided herein are methods for developing engineered non-alloreactive T-cells expressing the CAR for immunotherapy and more specifically for methods for increasing the persistence and/or the engraftment of allogeneic T cells.

In some embodiments, the T cell is genetically engineered by altering or modulating by genetic disruption one or more endogenous gene in the T cell. In some embodiments, the endogenous gene can be a gene sequence associated with host versus graft response or a gene sequence associated with graft versus host response. In some embodiments, the endogenous gene can be a gene sequence associated with a host versus graft response that is selected from the group consisting of B2M, CIITA, and RFX5, and combinations thereof. B2M is a common (invariant) component of MHC I complexes. CIITA and RFX5 are components of a transcription regulatory complex that is required for the expression of MHC II genes. Disrupting gene expression of these genes to eliminate their expression by gene editing can prevent host versus graft (e.g. T cell therapy) leading to increased allogeneic T cell persistence. In some embodiments, the endogenous gene can be a gene sequence associated with a graft versus host response that is selected from the group consisting of TRAC, CD3-epsilon (CD3ε), and combinations thereof. TRAC and CD3ε are components of the T cell receptor (TCR). Disrupting them by gene editing can take away the ability of the T cells to cause graft versus host disease.

In some cases, cells with reduced or eliminated cell-surface expression of MHC may become susceptible to NK cell-mediated cytotoxicity. In some aspects, certain MHC class I molecules, such as the non-classical MHC molecules MHC-E (HLA-E in humans and Qa-lb in mice) or MHC-G (HLA-G in humans), are ligands of and can be recognized by Natural Killer (NK) inhibitory receptors expressed on the surface of NK cells to induce an inhibitory signal to “stop” or halt an NK cell killing response. For example, MHC-E can interact with an inhibitor receptor on the surface of an NK cell that comprises CD94 and/or NKG2A, such as heterodimer of NKG2A disulfide-linked with the CD94 molecule. In some cases, an NK cell response can be triggered to kill cells that they interact with, unless those cells express the MHC molecule recognized by an NK inhibitory cell receptor on the NK cell. In cells in which MHC-I has been downregulated (e.g., as occurs in tumor or virally-infected cells), the NK cells can provide an immune surveillance by detection of “missing self,” which then results in cell killing. In the context of the provided embodiments, while reduced or disrupted expression of certain regulatory molecules (e.g. B2M) in the provided cells can reduce or eliminate expression of classical MHC class I (MHC class Ia) by the cell or on the cell surface, such targeting of regulatory molecules also may reduce or eliminate expression of non-classical MHC class I molecules MHC-E and MHC-G by the cell or on the cell surface, which also may render the cell susceptible to NK cell killing.

In some embodiments, to overcome or reduce risks of or associated with exposure to NK cell-mediated cytotoxicity, e.g., due to the reduced MHC expression, the provided engineered cells include those that are reduced or prevented from being the subject of “missing self” recognition, e.g., by NK cells, to prevent NK cell-mediated immune surveillance that could kill a provided engineered cell lacking an MHC molecule (e.g. MHC-E or MHC-G, also known as HLA-E or HLA-G, respectively). Thus, in some embodiments, in addition to reducing or eliminating expression of an MHC molecule (e.g. MHC class I), the provided engineered cells also include a recombinant NK cell modulator that is or comprises an NK cell modulating (e.g. inhibiting) moiety on the surface of the engineered immune cell. In some embodiments, the NK cell modulating (e.g. inhibiting) moiety is capable of inducing an inhibitory signal in an NK cell. In some embodiments, the binding or modulating induces an inhibitory signal in the NK cell to reduce or prevent lack-of-self recognition and NK cell-mediated rejection. In some embodiments, the inhibitory signal is transduced by a CD94/NKG2A receptor. In some embodiments, the modulating (e.g. inhibiting) moiety is a recombinant HLA-E molecule or binding portion thereof or a recombinant HLA-G molecule or binding portion thereof, for example, one that is exogenously introduced for expression on the surface of the cell. In some embodiments, such features of the provided cells result in enhanced efficacy or longevity of adoptive cell therapy in the context of engineered immune cells susceptibility to natural killer (NK) cell-mediated cytotoxicity.

In some embodiments, the engineered T cells are genetically engineered by expression of a CAR transgene, a genetic disruption that reduces or eliminates the expression or activity of a regulatory molecule that regulates expression and/or surface expression of an endogenous major histocompatibility complex (MHC) class I, a genetic disruption that reduces or eliminates the expression of TRAC, and also expression of an NK cell inhibiting moiety transgene. In some embodiments, the engineered T cells include a CAR transgene with an extracellular binding domain composed of a means for specifically binding an antigen or antigens, a genetic disruption that reduces or eliminates the expression of B2M to reduce or eliminate surface expression of endogenous MHC class I, a genetic disruption that reduces or eliminates the expression of TRAC, and a transgene for expression of a chimeric HLA-E transgene on the surface of the engineered T cell.

In some embodiments, the provided engineering strategies can be carried out by gene editing methods, including those involving CRISPR-Cas systems. In addition to disrupting or deleting genes by nuclease-directed targeted gene editing, introduction of transgenes (such as the CAR or NK cell inhibiting moiety) can be carried out by insertion into genomic loci at the site of a double stranded break that is repaired by homology directed repair (HDR) using a delivered donor template (e.g. by AAV delivery) with homology around the target site. Gene editing using rare-cutting endonucleases, such as CRISPR-Cas systems using guide RNA/Cas, to disrupt by knock-out (KO) target genes as well as to introduce by knock-in (KI) transgenes to a defined genomic loci has the benefit to modulate gene activity while also providing precise genome modification as compared to alternative methods such as lentivirus delivery and integration.

In some embodiments, the provided engineered T cells include a genetic disruption to inactivate or delete one or more genes implicated in the self/non-self-recognition (e.g., the TRAC and/or B2M gene) by the use of specific rare-cutting endonuclease, followed by a step of knock-in (KI) of said engineered T cells with at least one non-endogenous polypeptide transgene (such as HLA-E fusion protein and/or a recombinant CAR). In some embodiments, provided herein is a genetically engineered T cell with a genetic disruption in the endogenous TRAC gene; a genetic disruption in the endogenous B-2 microglobulin (B2M) gene; a nucleotide sequence encoding a single chain HLA-E fusion protein; and a nucleotide sequence encoding a chimeric antigen receptor (CAR). In some impairments, the genetic disruptions are by CRISPR-Cas systems using gRNAs useful for the creation of indels that result in disruption of the target gene, such as disruption of all alleles of the target gene, for example, reduction or elimination of gene expression and/or function. In some embodiments, the gRNAs are useful for the creation of double strand breaks (DSBs) that facilitate insertion of a donor template into the genome by HDR, In some embodiments, the CAR is integrated by KI into the disrupted TRAC gene by homology directed repair (HDR). In some embodiments, the single chain HLA-E fusion protein is integrated by KI into the disrupted B2M gene by homology directed repair (HDR).

Also provided herein are methods for engineering the T cells. Also provided herein are methods of administering the engineered T cells to a subject, such as for use in treating a disease or condition associated with expression of an antigen that is recognized by the recombinant receptor (e.g. CAR).

In some aspects, the provided engineered cells exhibit enhanced efficacy or longevity when used in adoptive cell therapy, for example, due to reduced or eliminated graft versus host rejection, host versus graft rejection and/or NK-cell mediated rejection. In some embodiments, the provided methods reduce or lessen or prevent an immune response in a subject administered with the genetically engineered T cells, compared to the immune response generated in the subject administered with T cells expressing the CAR in the absence of the genetic disruptions (e.g., KO of B2M and TRAC) and expression of an NK-cell inhibiting moiety transgene (e.g., HLA-E single chain fusion) in the engineered T cell. In some embodiments, the subject does not exhibit an immune response or a particular type or degree of immune response, against the genetically engineered T cells, such as following the administration of the cells to the subject. The type of immune response may be a detectable immune response, a humoral immune response, and/or a cell-mediated immune response. In some aspects, the provided genetically engineered T cells, compositions and methods result in an increased persistence and efficacy of cells used in adoptive cell therapy. In some aspects, the provided embodiments may reduce the number of T cells that need to be generated or delivered to each patient as the cells can be more efficacious and/or persist for longer. The provided embodiments may also reduce the number of sequential administrations of engineered cells required to treat a patient, or increase the amount of time needed between administrations as cells survive longer.

In some embodiments, the provided engineered cells, compositions and methods can be used regardless of the HLA type or subtype of a subject (e.g., a patient) to whom the cells may be administered, which can, in some aspects, permit “off-the-shelf” delivery to a wider variety of recipients. In some embodiments, the provided compositions and methods can be used to provide adoptive cell therapy using allogeneic cells engineered to treat a disease or disorder. In some cases, using allogeneic cells can provide certain advantages. In some embodiments, cells with known safety and efficacy profiles can be prepared for a wider variety of patients. For example, cells can be derived from a healthy donor and delivered to a subject that may be too sick to provide cells suitable for genetic engineering. In some cases, a subject may have a defect or disease in the cells or cell type typically used for a particular adoptive cell therapy regimen, such that cells from a healthy donor can be used that replace or supplement the diseased cells. In some cases, the ability to engineer or administer allogeneic cells permits the preparation of cells in advance, which can reduce the time needed before being delivered to a patient. In some cases, the engineered allogeneic cells may present lower risks of causing graft-versus-host disease or host-versus-graft disease.

Provided are T cells engineered according to the above provided strategies and engineered to express a CAR and compositions containing such cells. Also provided are approaches useful in the treatment of diseases and conditions and/or for targeting such cell types and compositions and articles of manufacture comprising the same.

Among the CAR in the provided genetically engineered T cells are chimeric antigen receptors (CARs) targeting or directed to CD19. Such CAR-engineered T cells can be used for targeting CD19-expressing cells, such as tumor cells associated with cancer. The CARs can contain antibodies (including antigen-binding antibody fragments, such as heavy chain variable (VH) regions, single domain antibody fragments and single chain fragments, including scFvs, and camelid-derived single domain antibody fragments such as VHH domains) specific for CD19. Also provided are cells, such as engineered or recombinant cells expressing such CD19-binding receptors, e.g., CARs and/or containing nucleic acids encoding such receptors, and compositions and articles of manufacture and therapeutic doses containing such cells.

Provided are cell therapy approaches utilizing CARs targeting CD19 expressed on autologous primary T cells for use as a therapeutic agent against cancer cells. In some cases, simultaneously targeting both antigens as provided herein may improve the depth and durability of responses across patients, in addition to minimizing relapse due to antigen escape. A mechanism of resistance to CAR T-cell therapies, as evidenced by data from CAR T-cell trials in B-cell malignancies, may be the loss or downregulation (“escape”) of the target antigen. (Robbie G. Majzner and Crystal L. Mackall, Cancer Discov Aug. 22 2018; DOI 10.1158/2159-8290.CD-18-0442). Such a dual targeting strategy may achieve synergistic or improved tumor responses based on targeting two antigens compared to approaches involving only single antigen targeting. A dual targeting approach may be advantageous to overcome problems due to potential for antigen loss and/or to maximize antigen targeting in cancer.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The Section headings used herein are for organizational purposes only and are not to be construed as limiting the subject matter described.

### I. CAR-ENGINEERED T CELLS AND METHODS OF MAKING THE SAME

Provided herein are genetically engineered T cells that express a recombinant chimeric antigen receptor (CAR), and that also are genetically engineered to have reduced or eliminated expression an endogenous major histocompatibility complex (MHC), e.g. MHC class I or MHC class II by genetic disruption of B2M, CIITA, RFX5, and combinations thereof; reduced or eliminated expression of TRAC or CD3-epsilon; and introduction of a transgene sequence to express a NK cell inhibiting moiety, such as an HLA-E or HLA-G. In some embodiments, the HLA-E or HLA-G are single chain fusion proteins with at least a portion of B2M to promote expression on the cell surface.

In some embodiments, the provided engineered T cells comprise a genetic disruption at a target site at an endogenous T cell receptor alpha constant (TRAC) locus, for example, to knock-out (KO) or reduce or eliminate the expression of the gene product of the TRAC locus. In some embodiments, the provided engineered T cells comprise a genetic disruption at a target site at Beta-2 microglobulin (B2M) locus, for example, to knock-out (KO) or reduce or eliminate the expression of the gene product of the B2M locus.

In some embodiments, the provided engineered T cells comprise a modified T cell receptor alpha constant (TRAC) locus comprising a transgene encoding the recombinant CAR, or portion thereof. In some aspects, the transgene (e.g., sequences that are exogenous or heterologous to the T cell) encoding the recombinant CAR or a portion thereof, is integrated at the TRAC locus of the T cell, by targeted knock-in (KI), and the expression of the endogenous TRAC gene product, the TCRa constant region, is reduced or eliminated. In some embodiments, the provided engineered T cells express a single chain HLA-E fusion transgene. In some aspects, the provided engineered T cells also comprise a modified Beta-2 microglobulin (B2M) locus comprising a transgene encoding the recombinant HLA-E fusion protein, or portion thereof. In some aspects, the transgene (e.g., sequences that are exogenous or heterologous to the T cell) encoding the recombinant HLA-E fusion protein or a portion thereof, is integrated at the B2M locus of the T cell, by targeted knock-in (KI), and the expression of the endogenous B2M gene product is reduced or eliminated.

In some aspects, the provided cells are engineered by CRISPR/Cas mediated gene editing to introduce a genetic disruption at a target site, and/or targeted integration (targeted knock-in, KI) of transgene sequences, for example encoding the recombinant CAR or HLA-E fusion protein, at or near one of the target sites with the genetic disruption. In some aspects, a genetic disruption is introduced at a target site at a TRAC or B2M locus, and in the presence of a polynucleotide comprising transgene sequences encoding a recombinant CAR or HLA-E fusion protein, respectively, or a portion thereof, the transgene sequences is integrated into a location at or near the target site with the genetic disruption, for example, by homology-directed repair (HDR). Exemplary methods for carrying out genetic disruptions at the endogenous loci and/or for carrying out HDR for targeted integration of the transgenes are described in this disclosure. Further, the engineered T cells can be generated using other methods, for example, as described in WO2015/161276, WO2015/070083, WO2019/070541, WO2019/195491, WO2019/195492, WO2019/089884, and WO2020/223535, the contents of which are incorporated by reference.

### A. Source T Cells

Any source of T cells can be used for genetically engineering T cells in accord with the provided embodiments. In some embodiments, the T cells are eukaryotic cells, such as mammalian cells, and typically are human cells. In some embodiments, the cells are derived from the blood, bone marrow, lymph, or lymphoid organs. In some embodiments, the T cells are derived such as differentiated from stem cells, such as multipotent and pluripotent stem cells, including induced pluripotent stem cells (iPSCs). The cells typically are primary cells, such as those isolated directly from a subject and/or isolated from a subject and frozen. In some embodiments, the T cells are from a healthy donor. In some embodiments, the healthy donor is age 18 to 35 years old. In some embodiments, a healthy donor has a body mass index (BMI) less than 30 kg/m². In some embodiments, a healthy donor is age 18 to 35 years old and has a body mass index (BMI) less than 30 kg/m².

In some embodiments, the cells include one or more subsets of T cells or other cell types, such as whole T cell populations, CD4′ cells, CD8′ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. In some embodiments, provided herein is a T cell comprising a provided bispecific CAR. In some embodiments the T cell is a CD4+ T cell. In some embodiments, the T cell is a CD8+ T cell.

Among the sub-types and subpopulations of T cells and/or of CD4+ and/or of CD8+ T cells are naïve T (T_{N}) cells, effector T cells (T_{EFF}), memory T cells and sub-types thereof, such as stem cell memory T (T_{SCM}), central memory T (T_{CM}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

With reference to the subject to be treated, the cells may be allogeneic and/or autologous. Typically, the cells are allogeneic to the subject being treated. Among the methods include off-the-shelf methods.

In some embodiments, the methods include isolating cells from the subject, preparing, processing, culturing, and/or engineering them, as described herein, and re-introducing them into the same patient, before or after cryopreservation.

In some embodiments, the cells include one or more polynucleotides introduced via genetic engineering, and thereby express recombinant or genetically engineered products of such polynucleotides. In some embodiments, the polynucleotides are heterologous, i.e., normally not present in a cell or sample obtained from the cell, such as one obtained from another organism or cell, which for example, is not ordinarily found in the cell being engineered and/or an organism from which such cell is derived. In some embodiments, the polynucleotides are not naturally occurring, such as a polynucleotide not found in nature, including one comprising chimeric combinations of polynucleotides encoding various domains from multiple different cell types. In some embodiments, the cells (e.g., engineered cells) comprise a vector (e.g., a viral vector, expression vector, etc.) as described herein such as a vector comprising a nucleic acid comprising a nucleic acid encoding a recombinant receptor described herein.

In some embodiments, T cells are isolated, selected, or enriched cells from a biological sample. In some embodiments, the biological sample is a sample from a donor subject (e.g. donor samples). In some embodiments, the donor subject is a subject that does not have a particular disease or condition or is not in need of a cell therapy or to which cell therapy will be administered. In some embodiments, the donor subject is a healthy subject or is believed to be a healthy subject (i.e. has not been diagnosed with a disease or condition).

In some embodiments, the T cells are primary T cells, such as primary human T cells. In some embodiments, the donor sample is a sample from an individual donor. In some embodiments, samples from a plurality of different individual donors are combined into a donor sample. In some aspects, the donor sample is from samples from a plurality of different individual donors. In some aspects, the donor sample is from a plurality of different donors. In some aspects, the individual donor is a human. In some aspects, each of the plurality of different donors is a human. In some aspects, the plurality of different donors are human donors. In some embodiments, the sample (e.g. donor sample) comprises primary human T cells from an individual donor. In some embodiments, each of the samples (e.g. donor samples) from a plurality of different individual donors are combined. In some embodiments, the sample (e.g. donor sample) comprises primary human T cells from a plurality of different donors. In some embodiments, the human donor is a healthy human donor.

In some embodiments, the samples include tissue, fluid, and other samples taken directly from the donor. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources. In some embodiments, the samples are from allogeneic sources (e.g. allogenic donors). In some embodiments, the samples are from autologous sources (e.g. autologous donors). In some embodiments, the sample is or comprises a whole blood sample, a buffy coat sample, a peripheral blood mononuclear cells (PBMC) sample, an unfractionated T cell sample, a lymphocyte sample, a white blood cell sample, an apheresis product, or a leukapheresis product.

In some examples, cells from the circulating blood of a donor are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

In some embodiments, the blood cells collected from the donor are washed, e.g., to remove the plasma fraction and to place the cells in an appropriate buffer or media for subsequent processing steps. In some embodiments, the sample containing cells (e.g., a donor sample, such as an apheresis product or a leukapheresis product) is washed in order to remove one or more anti-coagulants, such as heparin, added during apheresis or leukapheresis. In some embodiments, the cells are washed with phosphate buffered saline (PBS).

In some embodiments, the sample containing cells (e.g., donor sample, such as an apheresis product or a leukapheresis product) is cryopreserved and/or cryoprotected (e.g., frozen) and then thawed and optionally washed prior to any steps for isolating or selecting T cells or genetically engineering the cells.

In certain embodiments, subsets of T cells, e.g., CD3+, CD4+ or CD8+ T cells, are selected, isolated, or enriched from the donor sample or pooled donor samples. In some embodiments, CD4+ and CD8+ T cells are selected, isolated, or enriched the donor sample or pooled donor samples. In particular embodiments, CD3+ T cells are selected, isolated, or enriched the donor sample or pooled donor samples. In some embodiments, CD4+ and CD8+ are selected from a donor by adding CD4 and CD8 selection beads to a leukapheresis product from the donor. In some embodiments, CD8+ cells are selected first by adding CD8 selection beads to a leukapheresis product and keeping the CD8 selected fraction separate from the remaining leukapheresis product, then adding CD4 selection beads to the remaining leukapheresis product to yield a CD4 selected fraction. In some embodiments, the separate CD4+ and CD8+ fractions are mixed in a 1:1 ratio.

In some embodiments, selection, isolation, or enrichment includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components. In some embodiments, the methods include density-based cell separation methods, such as the preparation of white blood cells from peripheral blood by lysing the red blood cells and centrifugation through a Percoll or Ficoll gradient. In certain embodiments, methods, techniques, and reagents for selection, isolation, and enrichment are described, for example, in PCT Application Nos. WO2013124474 and WO2015164675, which are hereby incorporated by reference in their entirety.

In some embodiments, selection, isolation, or enrichment includes one or more selection steps. The selection can be a negative selection to deplete or remove unwanted cells or can be a positive selection of desired cells. In some embodiments, at least a portion of the selection step includes incubation of cells with a selection reagent. The incubation with a selection reagent or reagents, e.g., as part of selection methods which may be performed using one or more selection reagents for selection of one or more different cell types based on the expression or presence in or on the cell of one or more specific molecules, such as surface markers, e.g., surface proteins, intracellular markers, or nucleic acid. In certain embodiments, such surface proteins may include CD3, CD4, or CD8. In some embodiments, the selection reagent or reagents result in a separation that is affinity- or immunoaffinity-based separation.

In some embodiments, selected or isolated T cells are further enriched for naive, central memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in some aspects is particularly robust in such sub-populations. See Terakura et al., (2012) Blood. 1:72-82; Wang et al. (2012) J Immunother. 35(9):689-701. In certain embodiments, central memory T cells may include cells in various differentiation states and may be characterized by positive or high expression (e.g., surface expression) of certain cell markers and/or negative or low expression (e.g., surface expression) of other cell markers. In some aspects, less differentiated cells, e.g., central memory cells, are longer lived and exhaust less rapidly, thereby increasing persistence and durability. In some aspects, a responder to a cell therapy, such as a CAR-T cell therapy, has increased expression of central memory genes. See, e.g., Fraietta et al. (2018) Nat Med. 24(5):563-571. In some aspects, central memory T cells are characterized by positive or high expression of CD45RO, CD62L, CCR7, CD28, CD3, and/or CD127. In some aspects, central memory T cells are characterized by negative or low expression of CD45RA and/or granzyme B. In certain embodiments, central memory T cells or the T cells that are surface positive for a marker expressed on central memory T cells are CCR7+CD45RA−.

In some embodiments, “depleting” or “removing” when referring to one or more particular cell type or cell population, refers to decreasing the number or percentage of the cell type or population, e.g., compared to the total number of cells in or volume of the composition, or relative to other cell types, such as by negative selection based on markers expressed by the population or cell, or by positive selection based on a marker not present on the cell population or cell to be depleted. In general, the terms depleting or removing does not require complete removal of the cell, cell type, or population from the composition.

In some embodiments, “enriching” when referring to one or more particular cell type or cell population, refers to increasing the number or percentage of the cell type or population, e.g., compared to the total number of cells in or volume of the composition, or relative to other cell types, such as by positive selection based on markers expressed by the population or cell, or by negative selection based on a marker not present on the cell population or cell to be depleted. In general, the term enriching does not require complete removal of other cells, cell type, or populations from the composition and does not require that the cells so enriched be present at or even near 100% in the enriched composition.

Hence, it is understood that the separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some embodiments, the isolation and/or enrichment results in a population of enriched CD3+ T cells that includes at least 60%, at least 65%, at least 70%, at least 75%, at least 80%, at least 85%, at least 90%, at least 95%, at least 98%, at least 99%, at least 99.5%, at least 99.9%, or at or at about 100% CD3+ T cells.

In some embodiments, the isolation and/or enrichment results in a population of enriched CD4+ T cells that includes at least 60%, at least 65%, at least 70%, at least 75%, at least 80%, at least 85%, at least 90%, at least 95%, at least 98%, at least 99%, at least 99.5%, at least 99.9%, or at or at about 100% CD4+ T cells. In some embodiments, the isolation and/or enrichment results in a population of enriched CD8+ T cells that includes at least 60%, at least 65%, at least 70%, at least 75%, at least 80%, at least 85%, at least 90%, at least 95%, at least 98%, at least 99%, at least 99.5%, at least 99.9%, or at or at about 100% CD8+ T cells.

In some embodiments, the selected CD4+ cell population and the selected CD8+ cell population may be combined subsequent to the selecting. In some embodiments, the T cell population has a ratio of CD4+ to CD8+ T cells of between at or about 1:5 and at or about 5:1. In some embodiments, the T cell population has a ratio of CD4+ to CD8+ T cells of between at or about 1:3 and at or about 3:1. In some embodiments, the T cell population has a ratio of CD4+ to CD8+ T cells of between at or about 1:2 and at or about 2:1.

In some embodiments, the CD4+ and CD8+ T cells are activated by incubation with anti-CD3 and anti-CD28 antibodies. In some embodiments, CD4+ and CD8+ T cells are activated by culturing the T cells with a soluble anti-CD3/anti-CD28 Fab stimulatory reagent composed of the Fab agents on a streptavidin mutein backbone (see e.g., PCT publication No. WO2018/197949), followed by addition of D-Biotin to reversibly dissociate the Fab reagents from the backbone to disrupt the stimulation and washing off the stimulatory reagent. In some embodiments, CD4+ and CD8+ T cells are activated with Expamers as described in Poltorak, M. P., et al. Expamers: a new technology to control T cell activation. Sci Rep 10, 17832 (2020), which is incorporated by reference in its entirety. Expamer reagents comprise anti-CD3 and anti-CD28 antibody Fab fragments carrying the Twin-Strep-tag affinity tag and are used to functionalize polymerized Strep-Tactin (mutein of streptavidin) multimer backbones. D-biotin addition dissociates the Fab fragments from T cell surface.

### B. Genetic Disruption

In some embodiments, one or more genetic disruption is induced at one or more target sites in the T cell. In some aspects, during the engineering of the T cell, one or more genetic disruption is induced at one or more target sites in the T cell. In some aspects, the genetic disruptions at target sites at the endogenous TRAC or the B2M locus then result in targeted integration of the transgene sequences at or near that target site.

In some embodiments, one or more further targeted genetic disruptions is induced at the endogenous TRAC locus. In some embodiments, one or more further targeted genetic disruptions is induced at one or more target sites at or near the endogenous TRAC locus. In some embodiments, the genetic disruption is induced in an exon of the endogenous TRAC locus. In some embodiments, the genetic disruption is induced in an intron of the endogenous TRAC locus. In some aspects, the presence of the one or more further genetic disruption and a polynucleotide, e.g., a template polynucleotide that contains transgene sequences encoding a recombinant CAR or a portion thereof, can result in targeted integration of the transgene sequences at or near the one or more genetic disruption at the endogenous TRAC locus. In some aspects, such targeted integration produces a modified TRAC locus comprising a transgene encoding the recombinant CAR, or a portion of the recombinant CAR.

In some embodiments, one or more further targeted genetic disruptions is induced at the endogenous B2M locus. In some embodiments, one or more targeted genetic disruption is induced at one or more target sites at or near the endogenous B2M locus. In some embodiments, the genetic disruption is induced in an exon of the endogenous B2M locus. In some embodiments, the genetic disruption is induced in an intron of the endogenous B2M locus. In some aspects, the presence of the one or more further genetic disruption and a further polynucleotide, e.g., a template polynucleotide that contains transgene sequences encoding a recombinant HLA-E fusion protein or a portion thereof, can result in targeted integration of the transgene sequences at or near the one or more genetic disruption at the endogenous B2M locus. In some aspects, such targeted integration produces a modified B2M locus comprising a transgene encoding the recombinant HLA-E fusion protein, or a portion of the recombinant HLA-E fusion protein.

In some embodiments, genetic disruption results in a DNA break or a nick. In some embodiments, at the site of the DNA break, action of cellular DNA repair mechanisms can result in a knock-out (KO), an indel, an insertion, a missense or a frameshift mutation, such as a biallelic frameshift mutation, and/or a deletion of all or part of the gene. In some embodiments, the genetic disruption can be targeted to one or more exon of a gene or portion thereof, such as within the first or second exon. In some embodiments, a DNA binding protein or DNA-binding nucleic acid, which specifically binds to or hybridizes to the sequences at a region near one of the at least one target site(s), is used for targeted disruption. In some aspects, in the absence of exogenous template polynucleotides for HDR the disruption, the targeted genetic disruption results in an indel, a deletion, a mutation and/or an insertion within an exon of the gene.

In some embodiments, polynucleotides, e.g., template polynucleotides that include a transgene encoding a recombinant CAR, HLA-E fusion protein or a portion thereof, and homology sequences, can be introduced for targeted integration of the recombinant CAR-encoding transgene or the recombinant HLA-E fusion protein-encoding transgene at or near the sites of the genetic disruptions, for example a second or third target site at the TRAC and B2M loci, respectively, by HDR.

In some embodiments, the genetic disruption is carried by introducing one or more agent(s) capable of inducing a genetic disruption. In some embodiments, such agents comprise a DNA binding protein or DNA-binding nucleic acid that specifically binds to or hybridizes to the gene. In some embodiments, the agent comprises various components, such as a fusion protein comprising a DNA-targeting protein and a nuclease or an RNA-guided nuclease. In some embodiments, the agents can target one or more target sites, e.g., a first target site at a TRAC locus and/or a second target site at a B2M locus.

In some embodiments, the genetic disruption occurs at a target site (also referred to and/or known as “target position,” “target DNA sequence,” or “target location”). In some embodiments, target site is or includes a site on a target DNA (e.g., genomic DNA) that is modified by the one or more agent(s) capable of inducing a genetic disruption, e.g., a Cas molecule complexed with a gRNA that specifies the target site. For example, in some embodiments, the target site may include locations in the DNA, e.g., at an endogenous TRAC and/or B2M loci, where cleavage or DNA breaks occur. In some aspects, integration of nucleic acid sequences by HDR can occur at or near the target site or target sequence. In some embodiments, a target site can be a site between two nucleotides, e.g., adjacent nucleotides, on the DNA into which one or more nucleotides is added. The target site may comprise one or more nucleotides that are altered by a template polynucleotide. In some embodiments, the target site is within a target sequence (e.g., the sequence to which the gRNA binds). In some embodiments, a target site is upstream or downstream of a target sequence.

In some embodiments, genetic disruption results in a DNA break, such as a double-strand break (DSB) or a cleavage, or a nick, such as a single-strand break (SSB), at one or more target site in the genome. In some embodiments, at the site of the genetic disruption, e.g., DNA break or nick, action of cellular DNA repair mechanisms can result in knock-out, insertion, missense or frameshift mutation, such as a biallelic frameshift mutation, deletion of all or part of the gene; or, in the presence of a repair template, e.g., a template polynucleotide, can alter the DNA sequence based on the repair template, such as integration or insertion of the nucleic acid sequences, such as a transgene encoding all or a portion of a recombinant CAR and/or a recombinant HLA-E fusion protein, contained in the template. In some embodiments, the genetic disruption can be targeted to one or more exon of a gene or portion thereof. In some embodiments, the genetic disruption can be targeted near a desired site of targeted integration of exogenous sequences, e.g., transgene sequences encoding a recombinant CAR and/or a recombinant HLA-E fusion protein.

### 1. Target Site at an Endogenous TRAC Locus

In some aspects, the provided engineered T cells comprise a genetic disruption at the endogenous genes that encode one or more domains, regions and/or chains of the endogenous T cell receptor (TCR). In some embodiments, the genetic disruption is targeted at the endogenous gene locus that encodes TCRα. In some embodiments, the genetic disruption is targeted at the endogenous gene encoding TCRα constant domain (TRAC in humans).

In some aspects, a genetic disruption at the TRAC locus reduces expression of the gene product of the TRAC locus (e.g., endogenous TCR alpha chain constant region (Cα)) in the T cells. In some embodiments, the reduced expression of TRAC includes reduced expression of an endogenous TRAC mRNA. In some embodiments, the genetic disruption that reduces expression of TRAC includes reduced expression of an endogenous TCR alpha chain constant region (Cα) protein, the protein encoded by the TRAC mRNA. In some embodiments, the genetic disruption eliminates TRAC gene activity. In some embodiments, the genetic disruption includes inactivation or disruption of both alleles of the TRAC locus. In some embodiments, the genetic disruption includes inactivation or disruption of all alleles of the TRAC locus. In some embodiments, the genetic disruption comprises inactivation or disruption of all TRAC coding sequences in the cell. In some embodiments, the genetic disruption comprises an insertion of the transgene at the TRAC locus. In some embodiments, the genetic disruption comprises an indel at the TRAC locus. In some embodiments, the genetic disruption comprises an indel and results in a knock-out (KO) at the TRAC locus. In some embodiments, the TRAC indels can be detected or quantitated, among a population of engineered T cells, by PCR-based methods such as ddPCR. In some embodiments, the genetic disruption is a frameshift mutation or a deletion of a contiguous stretch of genomic DNA of the TRAC gene. In some embodiments, the TRAC gene is knocked out. In some aspects, the genetically engineered T cell does not encode a functional endogenous Cα polypeptide. In some aspects, the genetically engineered T cell does not encode an endogenous Cα polypeptide. In some aspects, the genetically engineered T cell does not encode a full length endogenous Cα polypeptide. In some aspects, the expression of an endogenous Cα polypeptide is reduced or eliminated in the genetically engineered T cell. In some aspects, the pairing of a TCRβ chain with a TCRα chain comprising an endogenous Cα is reduced or eliminated in the genetically engineered T cell. In some embodiments, the genetically engineered T cell has reduced expression of CD3 on the cell surface. In some embodiments, the genetically engineered T cell does not express detectable CD3 on the cell surface.

In some embodiments, the endogenous TCR Ca is encoded by the TRAC gene (IMGT nomenclature). An exemplary sequence of the human T cell receptor alpha chain constant domain (TRAC) gene locus is set forth in SEQ ID NO: 106 (NCBI Reference Sequence: NG_001332.3, TRAC). In certain embodiments, a genetic disruption is targeted at, near, or within a TRAC locus. In certain embodiments, a genetic disruption is targeted at, near, or within a TRAC locus. In particular embodiments, the genetic disruption is targeted at, near, or within an open reading frame of the TRAC locus. In certain embodiments, the genetic disruption is targeted at, near, or within an open reading frame that encodes a TCRα constant domain.

In humans, an exemplary genomic locus of TRAC comprises an open reading frame that contains 4 exons and 3 introns. An exemplary mRNA transcript of TRAC can span the sequence corresponding to coordinates Chromosome 14: 22,547,506-22,552,154, on the forward strand, with reference to human genome version GRCh38 (UCSC Genome Browser on Human December 2013 (GRCh38/hg38) Assembly). Table 1 sets forth the coordinates of the exons and introns of the open reading frames and the untranslated regions of the transcript of an exemplary human TRAC locus.

TABLE 1
Coordinates of exons and introns of exemplary human
TRAC locus (GRCh38, Chromosome 14, forward strand).

| | Start (GrCh38) | End (GrCh38) | Length |
|---|---|---|---|
| 5′ UTR and Exon 1 | 22,547,506 | 22,547,778 | 273 |
| Intron 1-2 | 22,547,779 | 22,549,637 | 1,859 |
| Exon 2 | 22,549,638 | 22,549,682 | 45 |
| Intron 2-3 | 22,549,683 | 22,550,556 | 874 |
| Exon 3 | 22,550,557 | 22,550,664 | 108 |
| Intron 3-4 | 22,550,665 | 22,551,604 | 940 |
| Exon 4 and 3′ UTR | 22,551,605 | 22,552,154 | 550 |

In some embodiments, the genetic disruption is targeted at or in close proximity to the beginning of the coding region (e.g., the early coding region, e.g., within 500 bp from the start codon or the remaining coding sequence, e.g., downstream of the first 500 bp from the start codon). In some embodiments, the genetic disruption is targeted at early coding region of a gene of interest, e.g., TRAC, including sequence immediately following a transcription start site, within a first exon of the coding sequence, or within 500 bp of the transcription start site (e.g., less than 500, 450, 400, 350, 300, 250, 200, 150, 100 or 50 bp), or within 500 bp of the start codon (e.g., less than 500, 450, 400, 350, 300, 250, 200, 150, 100 or 50 bp).

In some embodiments, the target site is within an exon of the endogenous TRAC locus. In certain embodiments, the target site is within an intron of the endogenous TRAC locus. In some aspects, the target site is within a regulatory or control element, e.g., a promoter, 5′ untranslated region (UTR) or 3′ UTR, of the TRAC locus. In certain embodiments, the target site is within an open reading frame of an endogenous TRAC locus. In particular embodiments, the target site is within an exon within the open reading frame of the TRAC locus.

In particular embodiments, the genetic disruption is targeted at or within an open reading frame of a gene or locus of interest, e.g., TRAC locus. In some embodiments, the genetic disruption is targeted at or within an intron within the open reading frame of a gene or locus of interest. In some embodiments, the genetic disruption is targeted within an exon within the open reading frame of the gene or locus of interest.

In particular embodiments, a genetic disruption is targeted at or within an intron. In certain embodiments, a genetic disruption is targeted at or within an exon. In some embodiments, a genetic disruption is targeted at or within an exon of a gene of interest, e.g., TRAC locus.

In some embodiments, a genetic disruption is targeted within an exon of the TRAC gene, open reading frame, or locus. In certain embodiments, the genetic disruption is within the first exon, second exon, third exon, or fourth exon of the TRAC gene, open reading frame, or locus. In particular embodiments, the genetic disruption is within the first exon of the TRAC gene, open reading frame, or locus. In some embodiments, the genetic disruption is within 500 base pairs (bp) downstream from the 5′ end of the first exon in the TRAC gene, open reading frame, or locus. In particular embodiments, the genetic disruption is between the most 5′ nucleotide of exon 1 and upstream of the most 3′ nucleotide of exon 1. In certain embodiments, the genetic disruption is within 400 bp, 350 bp, 300 bp, 250 bp, 200 bp, 150 bp, 100 bp, or 50 bp downstream from the 5′ end of the first exon in the TRAC gene, open reading frame, or locus. In particular embodiments, the genetic disruption is between 1 bp and 400 bp, between 50 and 300 bp, between 100 bp and 200 bp, or between 100 bp and 150 bp downstream from the 5′ end of the first exon in the TRAC gene, open reading frame, or locus, each inclusive. In certain embodiments, the genetic disruption is between 100 bp and 150 bp downstream from the 5′ end of the first exon in the TRAC gene, open reading frame, or locus, inclusive.

In some aspects, the target site is within an exon, such as exons corresponding to early coding regions. In some embodiments, the target site is within or in close proximity to exons corresponding to early coding region, e.g., exon 1, 2, 3, 4 or 5 of the open reading frame of the endogenous TRAC locus (such as described in Table 1 herein), or including sequence immediately following a transcription start site, within exon 1, 2, 3, 4 or 5, or within less than 500, 450, 400, 350, 300, 250, 200, 150, 100 or 50 bp of exon 1, 2, 3, 4 or 5. In some aspects, the target site is at or near exon 1 of the endogenous TRAC locus, e.g., within less than 500, 450, 400, 350, 300, 250, 200, 150, 100 or 50 bp of exon 1. In some embodiments, the target site is at or near exon 2 of the endogenous TRAC locus, or within less than 500, 450, 400, 350, 300, 250, 200, 150, 100 or 50 bp of exon 2. In some aspects, the target site is at or near exon 3 of the endogenous TRAC locus, e.g., within less than 500, 450, 400, 350, 300, 250, 200, 150, 100 or 50 bp of exon 3. In some aspects, the target site is at or near exon 4 of the endogenous TRAC locus, e.g., within less than 500, 450, 400, 350, 300, 250, 200, 150, 100 or 50 bp of exon 4. In some aspects, the target site is at or near exon 5 of the endogenous TRAC locus, e.g., within less than 500, 450, 400, 350, 300, 250, 200, 150, 100 or 50 bp of exon 5. In some aspects, the target site is within a regulatory or control element, e.g., a promoter, of the TRAC locus.

In certain embodiments, a genetic disruption is targeted at, near, or within a TRAC locus. In particular embodiments, the genetic disruption is targeted at, near, or within an open reading frame of the TRAC locus (such as described in Table 1 herein). In certain embodiments, the genetic disruption is targeted at, near, or within an open reading frame that encodes a TRAC. In some embodiments, the genetic disruption is targeted at, near, or within the TRAC locus (such as described in Table 1 herein), or a sequence having at or at least 70%, 75%, 80%, 85%, 90%, 95%, 97%, 98%, 99%, 99.5%, or 99.9% sequence identity to all or a portion, e.g., at or at least 500, 1,000, 1,500, 2,000, 2,500, 3,000, 3,500, or 4,000 contiguous nucleotides, of the TRAC locus (such as described in Table 1 herein).

In some embodiments, the genetic disruption is in a target site sequence in exon 1 of the TRAC gene. In some embodiments, the target site sequence in exon 1 of the endogenous TRAC gene is located within a TRAC genome region at contiguous positions within the hg38 genomic region chr14:22,547,506-22,547,778. In some embodiments, the target site sequence in exon 1 of the endogenous TRAC gene is located at hg38 genomic coordinates chr14:22,547,576-22,547,595. In some embodiments, the target site sequence in exon 1 of the endogenous TRAC gene has the sequence set forth in SEQ ID NO: 84, a contiguous portion thereof of at least 14 nucleotides (nt), or a complementary sequence of the foregoing. In some embodiments, the target site sequence is 12, 13, 14, 15, 16, 17, 18 or 20 contiguous nucleotides of SEQ ID NO: 84.

In some embodiments, a target site at the TRAC locus comprises SEQ ID NO: 84 (GAGAATCAAAATCGGTGAAT).

In some embodiments, the genetic disruption is by editing a genomic locus, e.g., a TRAC locus, with an RNA-guided nuclease. In some embodiments, the RNA-guided nuclease is a CRISPR/Cas nuclease. In some embodiments, the RNA-guided nuclease is Cas9. In some embodiments, the CRISPR-Cas system is a ribonucleoprotein (RNP) complex comprising the Cas9 protein and the gRNA.

In some embodiments, the nuclease is S. pyogenes Cas9 or N. meningitidis Cas9. In some embodiments, nuclease is S. pyogenes Cas9. Any of the targeting domains can be used with a S. pyogenes Cas9 molecule that generates a double stranded break (Cas9 nuclease) or a single-stranded break (Cas9 nickase). Cas9 molecules of, derived from, or based on the Cas9 proteins of other species listed herein can be used as well. In other words, while the much of the description herein uses S. pyogenes, S. aureus, N. meningitidis, and S. thermophilus Cas9 molecules, Cas9 molecules from the other species can replace them.

In some embodiments, a gRNA sequence comprises CRISPR (cr)RNA and trans-activating (tra) CRISPR (cr) RNA.

In some embodiments, for genetic disruption using a CRISPR/Cas based gene editing, a gRNA sequence that is or comprises a targeting domain sequence (in some cases also referred to as a spacer sequence) that can bind to and/or target a target site in the genome, e.g., a target site at a TRAC locus. A genome-wide gRNA database for CRISPR genome editing is publicly available, which contains exemplary single guide RNA (sgRNA) sequences targeting constitutive exons of genes in the human genome or mouse genome (see e.g., genescript.com/gRNA-database.html; see also, Sanjana et al. (2014) Nat. Methods, 11:783-4). In some aspects, the gRNA sequence is or comprises a sequence with minimal off-target binding to a non-target site or position.

In some embodiments, the spacer sequence is SEQ ID NO: 87, or a sequence having at or at least 70%, 75%, 80%, 85%, 90%, 95%, 97%, 98%, 99%, 99.5%, or 99.9% sequence identity to SEQ ID NO: 87. In some embodiments, the spacer sequence comprises a nucleic acid sequence set forth in SEQ ID NO: 87, or a contiguous portion thereof of at least 5 nucleotides (nt), 6, 7, 8, 9, 10, 11, 12, 13, 14, 15, 16, 17, 18, 19, 20, 21, 22, 23, 24, or 25 nt. In some embodiments, the spacer sequence comprises a nucleic acid sequence set forth in SEQ ID NO: 87, or a contiguous portion thereof of at least 12, at least 13, or at least 14 nt. In some embodiments, the spacer sequence is SEQ ID NO: 87.

In some embodiments, the target site that the targeting domain of the gRNA binds to or targets is located at an early coding region of a gene of interest, such as TRAC. Targeting of the early coding region can be used to genetic disruption (i.e., eliminate expression of) the gene of interest. In some embodiments, the early coding region of a gene of interest includes sequence immediately following a start codon (e.g., ATG), or within 500 bp of the start codon (e.g., less than 500, 450, 400, 350, 300, 250, 200, 150, 100, 50 bp, 40 bp, 30 bp, 20 bp, or 10 bp). In particular examples, the target nucleic acid is within 200 bp, 150 bp, 100 bp, 50 bp, 40 bp, 30 bp, 20 bp or 10 bp of the start codon. In some examples, the targeting domain of the gRNA is complementary, e.g., at least 80, 85, 90, 95, 98 or 99% complementary, e.g., fully complementary, to the target site or a complement of the target site, such as the target nucleic acid in the TRAC locus, or the targeting domain of the gRNA can bind to or hybridize to the target site or a complement of the target site.

In some embodiments, the gRNA can target a site at the TRAC locus near a desired site of targeted integration of transgene sequences, e.g., encoding a recombinant receptor. In some aspects, the gRNA can target a site based on the amount of sequences encoding the TRAC that is desired for expression in the cell expressing the recombinant receptor. In some aspects, the gRNA can target a site within an exon of the open reading frame of the endogenous TRAC locus. In some aspects, the gRNA can target a site within an intron of the open reading frame of the TRAC locus. In some aspects, the gRNA can target a site within a regulatory or control element, e.g., a promoter, of the TRAC locus. In some aspects, the target site at the TRAC locus that is targeted by the gRNA can be any target sites described herein. In some embodiments, the gRNA can target a site within or in close proximity to exons corresponding to early coding region, e.g., exon 1, 2, 3, 4 or 5 of the open reading frame of the endogenous TRAC locus, or including sequence immediately following a transcription start site, within exon 1, 2, 3, 4 or 5, or within less than 500, 450, 400, 350, 300, 250, 200, 150, 100 or 50 bp of exon 1, 2, 3, 4 or 5. In some embodiments, the gRNA can target a site at or near exon 2 of the endogenous TRAC locus, or within less than 500, 450, 400, 350, 300, 250, 200, 150, 100 or 50 bp of exon 2.

In some embodiments, for genetic disruption using a CRISPR/Cas based gene editing, a gRNA sequence further comprises a scaffold sequence that is responsible for Cas9 binding. In some embodiments, the scaffold sequence is SEQ ID NO: 88, or a sequence having at or at least 70%, 75%, 80%, 85%, 90%, 95%, 97%, 98%, 99%, 99.5%, or 99.9% sequence identity to SEQ ID NO: 88. In some embodiments, the scaffold sequence is SEQ ID NO: 88.

In a CRISPR-endonuclease system, a spacer sequence can be designed to hybridize to a target polynucleotide that is located 5′ of a PAM of the endonuclease used in the system. The spacer may perfectly match the target sequence or may have mismatches. Each endonuclease, e.g., Cas9 nuclease, has a particular PAM sequence that it recognizes in a target DNA. For example, S. pyogenes Cas9 recognizes a PAM that comprises the sequence 5′-NRG-3′, where R comprises either A or G, where N is any nucleotide and N is immediately 3′ of the target nucleic acid sequence targeted by the spacer sequence.

In some embodiments, the Cas9 molecule interacts with a gRNA molecule. In some embodiments, the gRNA targets for disruption the TRAC target site sequence GAGAATCAAAATCGGTGAAT (SEQ ID NO: 84) at the TRAC locus in which the gRNA includes the spacer sequence GAGAAUCAAAAUCGGUGAAU SEQ ID NO: 87 and a scaffold sequence (SEQ ID NO: 88) for S. pyogenes Cas9 (spCas9). In some embodiments, the gRNA includes base modifications. In some embodiments, the gRNA is modified by one or more modified nucleotides, wherein the one or more modified nucleotides are for increased stability of the gRNA. In some embodiments, the gRNA is SEQ ID NO: 82 or SEQ ID NO: 92, or a sequence having at or at least 70%, 75%, 80%, 85%, 90%, 95%, 97%, 98%, 99%, 99.5%, or 99.9% sequence identity to SEQ ID NO: 82 or SEQ ID NO: 92. In some embodiments, the gRNA sequence is SEQ ID NO: 82 or SEQ ID NO: 92.

In some aspects, a genetic disruption at the TRAC locus is introduced using a first agent comprising a zinc finger nuclease (ZFN), a TAL-effector nuclease (TALEN), or a CRISPR-Cas combination.

### 2. Target Site at an Endogenous B2M Locus

In some aspects, the provided engineered T cells comprise a genetic disruption at the endogenous genes that encode one or more domains, regions and/or chains of the endogenous Beta-2 microglobulin (B2M), for example, to knock-out (KO) or reduce or eliminate the expression of the gene product of the B2M locus.

B2M is a component of the Major Histocompatibility Complex (MHC) class I molecule, which would not assemble on the cell surface without B2M. Thus, knockout of B2M is a method of eliminating MHC class I molecules, which reduces GVHD when CAR T cells are administered to allogeneic patients.

In some aspects, a genetic disruption at the B2M locus reduces expression of the gene product of the B2M locus in the T cells. In some embodiments, the reduced expression of B2M includes reduced expression of an endogenous B2M mRNA. In some embodiments, the genetic disruption eliminates B2M gene activity. In some embodiments, the genetic disruption includes inactivation or disruption of both alleles of the B2M locus. In some embodiments, the genetic disruption includes inactivation or disruption of all alleles of the B2M locus. In some embodiments, the genetic disruption comprises inactivation or disruption of all B2M coding sequences in the cell. In some embodiments, the genetic disruption comprises an insertion of the transgene at the B2M locus. In some embodiments, the genetic disruption comprises an indel at the B2M locus. In some embodiments, the genetic disruption comprises an indel and results in a knock-out (KO) at the B2M locus. In some embodiments, the B2M indels can be detected or quantitated, among a population of engineered T cells, by PCR-based methods such as ddPCR. In some embodiments, the genetic disruption is a frameshift mutation or a deletion of a contiguous stretch of genomic DNA of the B2M gene. In some embodiments, the B2M gene is knocked out. In some embodiments, the genetically engineered T cell has reduced expression of one or more HLA class I molecules (e.g., HLA-A class I, HLA-B class I and/or HLA-C class I) on the cell surface, optionally wherein the genetically engineered cell has no detectable expression of one or more HLA class I molecules (e.g., HLA-A class I, HLA-B class I and/or HLA-C class I) on the cell surface. In some embodiments, the genetically engineered T cell has no detectable expression of HLA-A class I, HLA-B class I and HLA-C class I on the cell surface.

In some aspects, the genetically engineered T cell does not encode a functional endogenous B2M polypeptide. In some aspects, the genetically engineered T cell does not encode an endogenous B2M polypeptide. In some aspects, the genetically engineered T cell does not encode a full length endogenous B2M polypeptide. In some aspects, the expression of an endogenous B2M polypeptide is reduced or eliminated in the genetically engineered T cell.

In some embodiments, the endogenous Beta-2 microglobulin is encoded by the B2M gene (IMGT nomenclature). An exemplary sequence of the human B2M gene locus is set forth in SEQ ID NO: 93. In certain embodiments, a genetic disruption is targeted at, near, or within a B2M locus. In certain embodiments, a genetic disruption is targeted at, near, or within a B2M locus. In particular embodiments, the genetic disruption is targeted at, near, or within an open reading frame of the B2M locus.

An exemplary mRNA transcript of B2M can span the sequence corresponding to coordinates Chromosome 15: 44,711,517-44,718,145, on the forward strand, with reference to human genome version GRCh38 (UCSC Genome Browser on Human December 2013 (GRCh38/hg38) Assembly).

In some embodiments, the genetic disruption is targeted at or in close proximity to the beginning of the coding region (e.g., the early coding region, e.g., within 500 bp from the start codon or the remaining coding sequence, e.g., downstream of the first 500 bp from the start codon). In some embodiments, the genetic disruption is targeted at early coding region of a gene of interest, e.g., B2M, including sequence immediately following a transcription start site, within a second exon of the coding sequence, or within 500 bp of the transcription start site (e.g., less than 500, 450, 400, 350, 300, 250, 200, 150, 100 or 50 bp), or within 500 bp of the start codon (e.g., less than 500, 450, 400, 350, 300, 250, 200, 150, 100 or 50 bp).

In some embodiments, the target site is within an exon of the endogenous B2M locus. In certain embodiments, the target site is within an intron of the endogenous B2M locus. In some aspects, the target site is within a regulatory or control element, e.g., a promoter, 5′ untranslated region (UTR) or 3′ UTR, of the B2M locus. In certain embodiments, the target site is within an open reading frame of an endogenous B2M locus. In particular embodiments, the target site is within an exon within the open reading frame of the B2M locus.

In particular embodiments, the genetic disruption is targeted at or within an open reading frame of a gene or locus of interest, e.g., B2M locus. In some embodiments, the genetic disruption is targeted at or within an intron within the open reading frame of a gene or locus of interest. In some embodiments, the genetic disruption is targeted within an exon within the open reading frame of the gene or locus of interest.

In particular embodiments, the genetic disruption is within the second exon of the B2M gene, open reading frame, or locus. In some embodiments, the genetic disruption is within 500 base pairs (bp) downstream from the 5′ end of the second exon in the TRAC gene, open reading frame, or locus. In particular embodiments, the genetic disruption is between the most 5′ nucleotide of exon 2 and upstream of the most 3′ nucleotide of exon 2. In certain embodiments, the genetic disruption is within 400 bp, 350 bp, 300 bp, 250 bp, 200 bp, 150 bp, 100 bp, or 50 bp downstream from the 5′ end of the second exon in the B2M gene, open reading frame, or locus. In particular embodiments, the genetic disruption is between 1 bp and 400 bp, between 50 and 300 bp, between 100 bp and 200 bp, or between 100 bp and 150 bp downstream from the 5′ end of the second exon in the B2M gene, open reading frame, or locus, each inclusive. In certain embodiments, the genetic disruption is between 100 bp and 150 bp downstream from the 5′ end of the second exon in the B2M gene, open reading frame, or locus, inclusive.

In particular embodiments, a genetic disruption is targeted at or within an intron. In certain embodiments, a genetic disruption is targeted at or within an exon. In some embodiments, a genetic disruption is targeted at or within an exon of a gene of interest, e.g., B2M locus.

In some embodiments, the genetic disruption in the endogenous B2M gene is in a target site sequence in exon 2 of the B2M gene. In some embodiments, the target site sequence in exon 2 of the endogenous B2M gene is located within a B2M genome region at contiguous positions within hg38 the genomic region 44,715,423-44,715,701. In some embodiments, the target site sequence in exon 2 of the endogenous B2M gene is located at hg38 genomic coordinates chr15:44,715,614-44,715,634.

In some embodiments, the target site sequence in exon 2 of the endogenous B2M gene has the sequence set forth in SEQ ID NO: 85, a contiguous portion thereof of at least 14 nucleotides (nt), or a complementary sequence of the foregoing. In some embodiments, the target site sequence includes 12, 13, 14, 15, 16, 17, 18, 19, 20 or 21 contiguous nucleotides of SEQ ID NO: 85. In some embodiments, a target site at the B2M locus comprises SEQ ID NO: 85 (AGTGGGGGTGAATTCAGTGTA).

In some embodiments, the genetic disruption is by editing a genomic locus, e.g., a B2M locus, with an RNA-guided nuclease. In some embodiments, the RNA-guided nuclease is a CRISPR/Cas nuclease. In some embodiments, the RNA-guided nuclease is Cas12a (Cpf1). In some embodiments, the CRISPR-Cas system is a ribonucleoprotein (RNP) complex comprising the Cas12a protein and the gRNA.

In some embodiments, the nuclease is an Acidaminococcus sp. Cpf1 variant (AsCpf1 variant). Any of the targeting domains can be used with a Acidaminococcus sp. Cpf1 molecule that generates a double stranded break (Cas12a nuclease) or a single-stranded break (Cas12a nickase). Cas12a molecules of, derived from, or based on the Cas12a proteins of other species listed herein can be used as well. In other words, while much of the description herein uses Acidaminococcus sp. Cas12a, Cas12a molecules from the other species can replace them, such as Lachnospiraceae bacterium or Francisella novicida.

In some embodiments, a guide RNA (gRNA) sequence comprises CRISPR (cr)RNA. In some embodiments, the crRNA comprises a DNA extension to improve efficacy of the Cas12a activity. In some embodiments, the DNA extension is set forth in SEQ ID NO: 90.

In some embodiments, for genetic disruption using a CRISPR/Cas based gene editing, a gRNA sequence that is or comprises a targeting domain sequence (in some cases also referred to as a spacer sequence) that can bind to and/or target a target site in the genome, e.g., a target site at a B2M locus. A genome-wide gRNA database for CRISPR genome editing is publicly available, which contains exemplary guide RNA sequences targeting constitutive exons of genes in the human genome or mouse genome (see e.g., genescript.com/gRNA-database.html; see also, Sanjana et al. (2014) Nat. Methods, 11:783-4). In some aspects, the gRNA sequence is or comprises a sequence with minimal off-target binding to a non-target site or position.

In some embodiments, the spacer sequence is SEQ ID NO: 105, or a sequence having at or at least 70%, 75%, 80%, 85%, 90%, 95%, 97%, 98%, 99%, 99.5%, or 99.9% sequence identity to SEQ ID NO: 105. In some embodiments, the spacer sequence comprises a nucleic acid sequence set forth in SEQ ID NO: 105, or a contiguous portion thereof of at least 5 nucleotides (nt) 6, 7, 8, 9, 10, 11, 12, 13, 14, 15, 16, 17, 18, 19, 20, 21, 22, 23, 24, or 25 nt. In some embodiments, the spacer sequence comprises a nucleic acid sequence set forth in SEQ ID NO: 105, or a contiguous portion thereof of at least 12, at least 13, or at least 14 nt. In some embodiments, the spacer sequence is SEQ ID NO: 105.

In some embodiments, for genetic disruption using a CRISPR/Cas based gene editing, a gRNA sequence further comprises a scaffold sequence that is responsible for Cas12a binding. In some embodiments, the scaffold sequence is SEQ ID NO: 89, or a sequence having at or at least 70%, 75%, 80%, 85%, 90%, 95%, 97%, 98%, 99%, 99.5%, or 99.9% sequence identity to SEQ ID NO: 89. In some embodiments, the scaffold sequence is SEQ ID NO: 89.

A Cas12a molecule can interact with a gRNA molecule and, in concert with the gRNA molecule, homes or localizes to a site which comprises a target domain and PAM sequence.

In some embodiments, the Cas12a molecule interacts with a gRNA molecule. In some embodiments, the gRNA targets for disruption the B2M target site sequence AGTGGGGGTGAATTCAGTGTA (SEQ ID NO: 85 at the B2M locus in which the gRNA includes the DNA extension is set forth in SEQ ID NO: 90, the spacer sequence set forth in SEQ ID NO: 105, and the scaffold sequence set forth in SEQ ID NO: 89 for Cas12a. In some embodiments, the gRNA includes base modifications. In some embodiments, the gRNA is modified by one or more modified nucleotides, wherein the one or more modified nucleotides are for increased stability of the gRNA. In some embodiments, the gRNA is SEQ ID NO: 83, or a sequence having at or at least 70%, 75%, 80%, 85%, 90%, 95%, 97%, 98%, 99%, 99.5%, or 99.9% sequence identity to SEQ ID NO: 83. In some embodiments, the gRNA sequence is SEQ ID NO: 83.

In some aspects, a genetic disruption at the B2M locus is introduced using a first agent comprising a zinc finger nuclease (ZFN), a TAL-effector nuclease (TALEN), or a CRISPR-Cas combination.

### 3. Methods for Genetic Disruption

In some aspects, the methods for generating the genetically engineered cells involve introducing a genetic disruption at one or more target site(s), e.g., one or more target sites at a TRAC and/or B2M locus.

Methods for generating a genetic disruption, including those described herein, can involve the use of one or more agent(s) capable of inducing a genetic disruption, such as engineered systems to induce a genetic disruption, a cleavage and/or a double strand break (DSB) or a nick in a target site or target position in the endogenous DNA such that repair of the break by an error born process such as non-homologous end joining (NHEJ) or repair using a repair template HDR can result in the knock out of a gene and/or the insertion of a sequence of interest (e.g., exogenous nucleic acid sequences or transgene encoding a portion of a chimeric receptor) at or near the target site or position. Also provided are one or more agent(s) capable of inducing a genetic disruption, for example at one or more target sites described herein, for use in the methods provided herein. In some aspects, the one or more agent(s) can be used in combination with the template nucleotides provided herein, for homology directed repair (HDR) mediated targeted integration of the transgene sequences. Also provided are polynucleotides (e.g., nucleic acid molecules) encoding one or more components of the one or more agent(s) capable of inducing a genetic disruption.

In some aspects, the methods for generating the genetically engineered cells involve introducing a genetic disruption at a target site at a TRAC locus and/or a further target site at a B2M locus.

In some aspects, the genetic disruptions are introduced using one or more agents comprising a zinc finger nuclease (ZFN), a TAL-effector nuclease (TALEN), or a CRISPR-Cas combination. In some aspects, the one or more agents comprise a CRISPR-Cas combination comprising a guide RNA (gRNA) comprising a targeting domain that binds to the target site, and a Cas protein. In some aspects, the one or more agents comprise a first ribonucleoprotein (RNP) complex comprising the gRNA and the Cas protein.

In some embodiments, the one or more agent(s) specifically targets the at least one target site(s), e.g., a target site at a TRAC locus, a target site at a B2M locus, and/or a target site at a B2M locus. In some embodiments, the agent comprises a ZFN, TALEN or a CRISPR/Cas combination that specifically binds to, recognizes, or hybridizes to the target site(s). In some embodiments, the CRISPR/Cas system includes an engineered crRNA/tracr RNA (“single guide RNA”) to guide specific cleavage. In some embodiments, the CRISPR/Cas system does not include an engineered tracr RNA to guide specific cleavage. In some embodiments, the agent comprises nucleases based on the Argonaute system (e.g., from T. thermophilus, known as ‘TtAgo’, (Swarts et al. (2014) Nature 507(7491): 258-261). Targeted cleavage using any of the nuclease systems described herein can be exploited to insert the sequences of a transgene, e.g., nucleic acid sequences encoding a recombinant CAR or HLA-E fusion protein, into a specific target location, e.g., at a TRAC or at a B2M locus, using either HDR or NHEJ-mediated processes.

In some embodiments, the one or more agent(s) capable of inducing a genetic disruption comprises a DNA binding protein or DNA-binding nucleic acid that specifically binds to or hybridizes to a particular site or position in the genome, e.g., a target site or target position. In some aspects, the targeted genetic disruption, e.g., DNA break or cleavage, of the endogenous genes encoding TCR or B2M is achieved using a protein or a nucleic acid is coupled to or complexed with a gene editing nuclease, such as in a chimeric or fusion protein. In some embodiments, the one or more agent(s) capable of inducing a genetic disruption comprises an RNA-guided nuclease, or a fusion protein comprising a DNA-targeting protein and a nuclease.

In some embodiments, the agent comprises various components, such as an RNA-guided nuclease, or a fusion protein comprising a DNA-targeting protein and a nuclease. In some embodiments, the targeted genetic disruption is carried out using a DNA-targeting molecule that includes a DNA-binding protein such as one or more zinc finger protein (ZFP) or transcription activator-like effectors (TALEs), fused to a nuclease, such as an endonuclease. In some embodiments, the targeted genetic disruption is carried out using RNA-guided nucleases such as a clustered regularly interspaced short palindromic nucleic acid (CRISPR)-associated nuclease (Cas) system. In some embodiments, the targeted genetic disruption is carried using agents capable of inducing a genetic disruption, such as sequence-specific or targeted nucleases, including DNA-binding targeted nucleases and gene editing nucleases such as zinc finger nucleases (ZFN) and transcription activator-like effector nucleases (TALENs), and RNA-guided nucleases such as a CRISPR-associated nuclease (Cas) system, specifically designed to be targeted to the at least one target site(s), sequence of a gene or a portion thereof. Exemplary ZFNs, TALEs, and TALENs are described in, e.g., Lloyd et al., Frontiers in Immunology, 4(221): 1-7 (2013).

Various methods and compositions for targeted cleavage of genomic DNA have been described. Such targeted cleavage events can be used, for example, to induce targeted mutagenesis, induce targeted deletions of cellular DNA sequences, and facilitate targeted recombination at a predetermined chromosomal locus. See, e.g., U.S. Pat. Nos. 9,255,250; 9,200,266; 9,045,763; 9,005,973; 9,150,847; 8,956,828; 8,945,868; 8,703,489; 8,586,526; 6,534,261; 6,599,692; 6,503,717; 6,689,558; 7,067,317; 7,262,054; 7,888,121; 7,972,854; 7,914,796; 7,951,925; 8,110,379; 8,409,861; U.S. Patent Publications 20030232410; 20050208489; 20050026157; 20050064474; 20060063231; 20080159996; 201000218264; 20120017290; 20110265198; 20130137104; 20130122591; 20130177983; 20130196373; 20140120622; 20150056705; 20150335708; 20160030477 and 20160024474, the disclosures of which are incorporated by reference in their entireties.

In some embodiments, the agent comprises various components, such as an RNA-guided nuclease, or a fusion protein comprising a DNA-targeting protein and a nuclease. In some embodiments, the targeted genetic disruption is carried out using a DNA-targeting molecule that includes a DNA-binding protein such as one or more zinc finger protein (ZFP) or transcription activator-like effectors (TALEs), fused to a nuclease, such as an endonuclease. In some embodiments, the targeted genetic disruption is carried out using RNA-guided nucleases such as a clustered regularly interspaced short palindromic nucleic acid (CRISPR)-associated nuclease (Cas) system (including Cas9). In some embodiments, the targeted genetic disruption is carried using agents capable of inducing a genetic disruption, such as sequence-specific or targeted nucleases, including DNA-binding targeted nucleases and gene editing nucleases such as zinc finger nucleases (ZFN) and transcription activator-like effector nucleases (TALENs), and RNA-guided nucleases such as a CRISPR-associated nuclease (Cas) system, specifically designed to be targeted to the at least one target site(s), sequence of a gene or a portion thereof. Exemplary ZFNs, TALEs, and TALENs are described in, e.g., Lloyd et al., Frontiers in Immunology, 4(221): 1-7 (2013).

Various methods and compositions for targeted cleavage of genomic DNA have been described. Such targeted cleavage events can be used, for example, to induce targeted mutagenesis, induce targeted deletions of cellular DNA sequences, and facilitate targeted recombination at a predetermined chromosomal locus. See, e.g., U.S. Pat. Nos. 9,255,250; 9,200,266; 9,045,763; 9,005,973; 9,150,847; 8,956,828; 8,945,868; 8,703,489; 8,586,526; 6,534,261; 6,599,692; 6,503,717; 6,689,558; 7,067,317; 7,262,054; 7,888,121; 7,972,854; 7,914,796; 7,951,925; 8,110,379; 8,409,861; U.S. Patent Publications 20030232410; 20050208489; 20050026157; 20050064474; 20060063231; 20080159996; 201000218264; 20120017290; 20110265198; 20130137104; 20130122591; 20130177983; 20130196373; 20140120622; 20150056705; 20150335708; 20160030477 and 20160024474, the disclosures of which are incorporated by reference in their entireties.

A designed protein is a protein not occurring in nature whose design/composition results principally from rational criteria. Rational criteria for design include application of substitution rules and computerized algorithms for processing information in a database storing information of existing ZFP or TALE designs (canonical and non-canonical RVDs) and binding data. See, for example, U.S. Pat. Nos. 9,458,205; 8,586,526; 6,140,081; 6,453,242; and 6,534,261; see also WO 98/53058; WO 98/53059; WO 98/53060; WO 02/016536 and WO 03/016496.

Zinc finger proteins (ZFPs), transcription activator-like effectors (TALEs), and CRISPR system binding domains can be “engineered” to bind to a predetermined nucleotide sequence, for example via engineering (altering one or more amino acids) of the recognition helix region of a naturally occurring ZFP or TALE protein. Engineered DNA binding proteins (ZFPs or TALEs) are proteins that are non-naturally occurring. Rational criteria for design include application of substitution rules and computerized algorithms for processing information in a database storing information of existing ZFP and/or TALE designs and binding data. See, e.g., U.S. Pat. Nos. 6,140,081; 6,453,242; and 6,534,261; see also WO 98/53058; WO 98/53059; WO 98/53060; WO 02/016536 and WO 03/016496 and U.S. Publication No. 20110301073.

In some cases, the DNA-targeting molecule is or comprises a zinc-finger DNA binding domain fused to a DNA cleavage domain to form a zinc-finger nuclease (ZFN). For example, fusion proteins comprise the cleavage domain (or cleavage half-domain) from at least one Type IIS restriction enzyme and one or more zinc finger binding domains, which may or may not be engineered. In some cases, the cleavage domain is from the Type IIS restriction endonuclease FokI, which generally catalyzes double-stranded cleavage of DNA, at 9 nucleotides from its recognition site on one strand and 13 nucleotides from its recognition site on the other. See, e.g., U.S. Pat. Nos. 5,356,802; 5,436,150 and 5,487,994; Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269: 978-982. Some gene-specific engineered zinc fingers are available commercially. For example, a platform called CompoZr, for zinc-finger construction is available that provides specifically targeted zinc fingers for thousands of targets. See, e.g., Gaj et al., Trends in Biotechnology, 2013, 31(7), 397-405. In some cases, commercially available zinc fingers are used or are custom designed.

In some embodiments, the one or more target site(s), e.g., at a second target site at the TRAC locus genes can be targeted for genetic disruption by engineered ZFNs. Exemplary ZFN that target endogenous T cell receptor (TCR) genes include those described in, e.g., US 2015/0164954, US 2011/0158957, US 2015/0056705, U.S. Pat. No. 8,956,828 and Torikawa et al. (2012) Blood 119:5697-5705, the disclosures of which are incorporated by reference in their entireties.

Transcription Activator like Effector (TALE) are proteins from the bacterial species Xanthomonas comprise a plurality of repeated sequences, each repeat comprising di-residues in position 12 and 13 (RVD) that are specific to each nucleotide base of the nucleic acid targeted sequence. Binding domains with similar modular base-per-base nucleic acid binding properties (MBBBD) can also be derived from different bacterial species. In some embodiments, a “TALE DNA binding domain” or “TALE” is a polypeptide comprising one or more TALE repeat domains/units. The repeat domains, each comprising a repeat variable diresidue (RVD), are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. TALE proteins may be designed to bind to a target site using canonical or non-canonical RVDs within the repeat units. See, e.g., U.S. Pat. Nos. 8,586,526 and 9,458,205.

In some embodiments, a “TALE-nuclease” (TALEN) is a fusion protein comprising a nucleic acid binding domain typically derived from a Transcription Activator Like Effector (TALE) and a nuclease catalytic domain that cleaves a nucleic acid target sequence. The catalytic domain comprises a nuclease domain or a domain having endonuclease activity, like for instance I-TevI, ColE7, NucA and Fok-I. In a particular embodiment, the TALE domain can be fused to a meganuclease like for instance I-CreI and I-OnuI or functional variant thereof. In some embodiments, the TALEN is a monomeric TALEN. A monomeric TALEN is a TALEN that does not require dimerization for specific recognition and cleavage, such as the fusions of engineered TAL repeats with the catalytic domain of I-TevI described in WO2012138927. TALENs have been described and used for gene targeting and gene modifications (see, e.g., Boch et al. (2009) Science 326(5959): 1509-12.; Moscou and Bogdanove (2009) Science 326(5959): 1501; Christian et al. (2010) Genetics 186(2): 757-61; Li et al. (2011) Nucleic Acids Res 39(1): 359-72). In some embodiments, the TGFBR2 and/or TRAC genes can be targeted for genetic disruption by engineered TALENs. Exemplary TALEN that target endogenous T cell receptor (TCR) genes include those described in, e.g., WO 2017/070429, WO 2015/136001, US20170016025 and US20150203817, the disclosures of which are incorporated by reference in their entireties.

In some embodiments, a “TtAgo” is a prokaryotic Argonaute protein thought to be involved in gene silencing. TtAgo is derived from the bacteria Thermus thermophilus. See, e.g. Swarts et al, (2014) Nature 507(7491): 258-261, Sheng et al., (2013) Proc. Natl. Acad. Sci. U.S.A. 111, 652). A “TtAgo system” is all the components required including e.g. guide DNAs for cleavage by a TtAgo enzyme.

In some embodiments, an engineered zinc finger protein, TALE protein or CRISPR/Cas system is not found in nature and whose production results primarily from an empirical process such as phage display, interaction trap or hybrid selection. See e.g., U.S. Pat. Nos. 5,789,538; 5,925,523; 6,007,988; 6,013,453; 6,200,759; WO 95/19431; WO 96/06166; WO 98/53057; WO 98/54311; WO 00/27878; WO 01/60970; WO 01/88197 and WO 02/099084.

In some embodiments, the targeted genetic disruption of the endogenous genes such as TRAC and/or B2M in humans is carried out using clustered regularly interspaced short palindromic repeats (CRISPR) and CRISPR-associated (Cas) proteins. See Sander and Joung, (2014) Nature Biotechnology, 32(4): 347-355.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a targeting domain sequence (also referred to as a “spacer” in the context of an endogenous CRISPR system), and/or other sequences and transcripts from a CRISPR locus.

In some aspects, the CRISPR/Cas nuclease or CRISPR/Cas nuclease system includes a non-coding guide RNA (gRNA), which sequence-specifically binds to DNA, and a Cas protein (e.g., Cas9 or Cas12a), with nuclease functionality.

In some embodiments, the one or more agent(s) comprises a guide RNA (gRNA), having a targeting domain that binds to and/or is complementary with a target site at a TRAC gene or a complement thereof. In some embodiments, the one or more agent(s) comprises a further guide RNA (gRNA) having a targeting domain that binds to and/or is complementary with a target site at a B2M gene or a complement thereof.

In some aspects, a “gRNA molecule” is to a nucleic acid that promotes the specific targeting or homing of a gRNA molecule/Cas molecule complex to a target nucleic acid, such as a locus on the genomic DNA of a cell. gRNA molecules can be unimolecular (having a single RNA molecule), sometimes referred to herein as “chimeric” gRNAs, or modular (comprising more than one, and typically two, separate RNA molecules). In general, a guide sequence, e.g., guide RNA, is any polynucleotide sequences comprising at least a sequence portion that has sufficient complementarity with a target polynucleotide sequence, such as the TRAC and/or B2M genes in humans, to hybridize with the target sequence at the target site and direct sequence-specific binding of the CRISPR complex to the target sequence. In some embodiments, in the context of formation of a CRISPR complex, “target sequence” generally refers to a sequence to which a guide sequence is designed to have complementarity, where hybridization between the target sequence and a domain, e.g., targeting domain, of the guide RNA promotes the formation of a CRISPR complex. Full complementarity is not necessarily required, provided there is sufficient complementarity to cause hybridization and promote formation of a CRISPR complex. Generally, a guide sequence is selected to reduce the degree of secondary structure within the guide sequence. Secondary structure may be determined by any suitable polynucleotide folding algorithm.

In some embodiments, a guide RNA (gRNA) specific to a target locus of is used to RNA-guided nucleases, e.g., Cas, to induce a DNA break at the target site or target position. Methods for designing gRNAs and exemplary targeting domains can include those described in, e.g., WO2015/161276, WO2017/193107, WO2017/093969, US2016/272999 and US2015/056705, the contents of which are incorporated by reference. Methods for introducing a genetic disruption at one or more target sites and gRNAs that target the target sites include those described in, e.g., WO2015/161276, WO2015/070083, WO2019/070541, WO2019/195491, WO2019/195492, WO2019/089884, and WO2020/223535, the contents of which are incorporated by reference.

Several exemplary gRNA structures, with domains indicated thereon, are described in WO2015/161276. While not wishing to be bound by theory, with regard to the three dimensional form, or intra- or inter-strand interactions of an active form of a gRNA, regions of high complementarity are sometimes shown as duplexes in WO2015/161276.

In some cases, the gRNA is a unimolecular or chimeric gRNA comprising, from 5′ to 3′: a targeting domain which targets a target site (e.g., at the TRAC locus and/or the B2M locus); a first complementarity domain; a linking domain; a second complementarity domain (which is complementary to the first complementarity domain); a proximal domain; and optionally, a tail domain.

In other cases, the gRNA is a modular gRNA comprising first and second strands. In these cases, the first strand preferably includes, from 5′ to 3′: a targeting domain (which targets a target site); and a first complementarity domain. The second strand generally includes, from 5′ to 3′: optionally, a 5′ extension domain; a second complementarity domain; a proximal domain; and optionally, a tail domain.

Examples of the placement of targeting domains include those described in WO2015/161276. The targeting domain comprises a nucleotide sequence that is complementary, e.g., at least 80, 85, 90, 95, 98 or 99% complementary, e.g., fully complementary, to the target sequence on the target nucleic acid. The strand of the target nucleic acid comprising the target sequence is referred to herein as the “complementary strand” of the target nucleic acid. Guidance on the selection of targeting domains can be found, e.g., in Fu Y et al., Nat Biotechnol 2014 (doi: 10.1038/nbt.2808) and Sternberg S H et al., Nature 2014 (doi: 10.1038/nature13011). In some examples, the targeting domain of the gRNA is complementary, e.g., at least 80, 85, 90, 95, 98 or 99% complementary, e.g., fully complementary, to the target site.

The targeting domain is part of an RNA molecule and will therefore comprise the base uracil (U), while any DNA encoding the gRNA molecule will comprise the base thymine (T). While not wishing to be bound by theory, in some embodiments, it is believed that the complementarity of the targeting domain with the target sequence contributes to specificity of the interaction of the gRNA molecule/Cas molecule complex with a target nucleic acid. It is understood that in a targeting domain and target sequence pair, the uracil bases in the targeting domain will pair with the adenine bases in the target sequence. In some embodiments, the target domain itself comprises in the 5′ to 3′ direction, an optional secondary domain, and a core domain. In some embodiments, the core domain is fully complementary with the target sequence. In some embodiments, the targeting domain is 5 to 50 nucleotides in length. The strand of the target nucleic acid with which the targeting domain is complementary is referred to herein as the complementary strand. Some or all of the nucleotides of the domain can have a modification, e.g., to render it less susceptible to degradation, improve bio-compatibility, etc. By way of non-limiting example, the backbone of the target domain can be modified with a phosphorothioate, or other modification(s). In some cases, a nucleotide of the targeting domain can comprise a 2′ modification, e.g., a 2-acetylation, e.g., a 2′ methylation, or other modification(s).

In various embodiments, the targeting domain is 16-26 nucleotides in length (i.e. it is 16 nucleotides in length, or 17 nucleotides in length, or 18, 19, 20, 21, 22, 23, 24, 25 or 26 nucleotides in length.

### C. Targeted Integration Via Homology-Directed Repair (HDR)

In some of the embodiments provided herein, homology-directed repair (HDR) can be utilized for targeted integration of a specific portion of the template polynucleotide containing a transgene, e.g., nucleic acid sequence encoding a recombinant CAR or a NK cell inhibitory moiety (e.g., recombinant HLA-E fusion protein). In some embodiments, the targeted integration is at a particular location in the genome, e.g., the TRAC locus or the B2M locus.

In some embodiments, homology-directed repair (HDR) can be utilized for targeted integration or insertion of one or more nucleic acid sequences, e.g., transgene sequences, at one or more target site(s) in the genome. In some embodiments, the nuclease-induced HDR can be used to alter a target sequence, integrate a transgene at a particular target location, and/or to edit or repair a mutation in a particular target gene.

Alteration of nucleic acid sequences at the target site can occur by HDR with an exogenously provided polynucleotide (also referred to as donor polynucleotide or template sequence). For example, the template polynucleotide provides for alteration of the target sequence, such as insertion of the transgene contained within the template polynucleotide. In some embodiments, a plasmid or a vector can be used as a template for homologous recombination. In some embodiments, a linear DNA fragment can be used as a template for homologous recombination. In some embodiments, a single stranded template polynucleotide can be used as a template for alteration of the target sequence by alternate methods of homology directed repair (e.g., single strand annealing) between the target sequence and the template polynucleotide. Template polynucleotide-effected alteration of a target sequence depends on cleavage by a nuclease, e.g., a targeted nuclease such as CRISPR/Cas. Cleavage by the nuclease can comprise a double strand break or two single strand breaks.

In some embodiments, “recombination” refers to a process of exchange of genetic information between two polynucleotides. In some embodiments, “homologous recombination (HR)” refers to the specialized form of such exchange that takes place, for example, during repair of double-strand breaks in cells via homology-directed repair mechanisms. This process requires nucleotide sequence homology, uses a template polynucleotide to template repair of a target DNA (i.e., the one that experienced the double-strand break, e.g., target site in the endogenous gene), and is variously known as “non-crossover gene conversion” or “short tract gene conversion,” because it leads to the transfer of genetic information from the template polynucleotide to the target. In some embodiments, such transfer can involve mismatch correction of heteroduplex DNA that forms between the broken target and the template polynucleotide, and/or “synthesis-dependent strand annealing,” in which the template polynucleotide is used to resynthesize genetic information that will become part of the target, and/or related processes. Such specialized HR often results in an alteration of the sequence of the target molecule such that part or all of the sequence of the template polynucleotide is incorporated into the target polynucleotide. As described herein, the genetic disruption of the target site or target position can be created by any mechanisms, such as ZFNs, TALENs, CRISPR/Cas system, e.g., CRISPR/Cas9 or CRISPR/Cas12a, or TtAgo nucleases.

In some embodiments, double strand cleavage is affected by a nuclease, e.g., a Cas molecule having cleavage activity associated with an HNH-like domain and cleavage activity associated with a RuvC-like domain, e.g., an N-terminal RuvC-like domain, e.g., a wild type Cas nuclease.

In some embodiments, DNA repair mechanisms can be induced by a nuclease after (1) a single double-strand break, (2) two single strand breaks, (3) two double stranded breaks with a break occurring on each side of the target site, (4) one double stranded break and two single strand breaks with the double strand break and two single strand breaks occurring on each side of the target site (5) four single stranded breaks with a pair of single stranded breaks occurring on each side of the target site, or (6) one single stranded break. In some embodiments, a single-stranded template polynucleotide is used and the target site can be altered by alternative HDR.

Template polynucleotide-effected alteration of a target site depends on cleavage by a nuclease molecule. Cleavage by the nuclease can comprise a nick, a double strand break, or two single strand breaks, e.g., one on each strand of the DNA at the target site. After introduction of the breaks on the target site, resection occurs at the break ends resulting in single stranded overhanging DNA regions.

In canonical HDR, a double-stranded template polynucleotide is introduced, comprising homologous sequence to the target site that will either be directly incorporated into the target site or used as a template to insert the transgene or correct the sequence of the target site. After resection at the break, repair can progress by different pathways, e.g., by the double Holliday junction model (or double strand break repair, DSBR, pathway) or the synthesis-dependent strand annealing (SDSA) pathway.

In some embodiments, other DNA repair pathways such as single strand annealing (SSA), single-stranded break repair (SSBR), mismatch repair (MMR), base excision repair (BER), nucleotide excision repair (NER), intrastrand cross-link (ICL), translesion synthesis (TLS), error-free postreplication repair (PRR) can be employed by the cell to repair a double-stranded or single-stranded break created by the nucleases.

In some embodiments, one or more different template polynucleotides are used for targeting integration of the transgene at one or more different target sites. For targeting integration at different target sites, one or more genetic disruptions (e.g., DNA break) are generated at one or more of the target sites; and one or more different homology sequences are used for targeting integration of the transgene into the respective target site. In some embodiments, the transgene inserted at each site is the same or substantially the same. In some embodiments, transgene inserted at each site are different. In some embodiments, two or more different transgenes, encoding two or more different domains or chains of a protein, is inserted at one or more target sites.

The sequence of interest in the template polynucleotide may comprise one or more sequences encoding a functional polypeptide (e.g., a cDNA), with or without a promoter.

In some embodiments, nuclease-induced HDR results in an insertion of a transgene (also called “exogenous sequence” or “transgene sequence”) for expression of a transgene for targeted insertion. The template polynucleotide sequence is typically not identical to the genomic sequence where it is placed. A template polynucleotide sequence can contain a non-homologous sequence flanked by two regions of homology to allow for efficient HDR at the location of interest. Additionally, template polynucleotide sequence can comprise a vector molecule containing sequences that are not homologous to the region of interest in cellular chromatin. A template polynucleotide sequence can contain several, discontinuous regions of homology to cellular chromatin. For example, for targeted insertion of sequences not normally present in a region of interest, said sequences can be present in a transgene and flanked by regions of homology to sequence in the region of interest.

In some aspects, nucleic acid sequences of interest, including coding and/or non-coding sequences and/or partial coding sequences, that are inserted or integrated at the target location in the genome can also be referred to as “transgene,” “transgene sequences,” “exogenous nucleic acids sequences,” “heterologous sequences” or “donor sequences.” In some aspects, the transgene is a nucleic acid sequence that is exogenous or heterologous to an endogenous genomic sequences, such as the endogenous genomic sequences at a specific target locus or target location in the genome, of a T cell, e.g., a human T cell. In some aspects, the transgene is a sequence that is modified or different compared to an endogenous genomic sequence at a target locus or target location of a T cell, e.g., a human T cell. In some aspects, the transgene is a nucleic acid sequence that originates from or is modified compared to nucleic acid sequences from different genes, species and/or origins. In some aspects, the transgene is a sequence that is derived from a sequence from a different locus, e.g., a different genomic region or a different gene, of the same species.

Polynucleotides for insertion can also be referred to as “transgene” or “exogenous sequences” or “donor” polynucleotides or molecules. The template polynucleotide can be DNA, single-stranded and/or double-stranded and can be introduced into a cell in linear or circular form. The template polynucleotide can be RNA single-stranded and/or double-stranded and can be introduced as a RNA molecule (e.g., part of an RNA virus). See also, U.S. Patent Publication Nos. 20100047805 and 20110207221. The template polynucleotide can also be introduced in DNA form, which may be introduced into the cell in circular or linear form. If introduced in linear form, the ends of the template polynucleotide can be protected (e.g., from exonucleolytic degradation) by known methods. For example, one or more dideoxynucleotide residues are added to the 3′ terminus of a linear molecule and/or self-complementary oligonucleotides are ligated to one or both ends. See, for example, Chang et al. (1987) Proc. Natl. Acad. Sci. USA 84:4959-4963; Nehls et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. If introduced in double-stranded form, the template polynucleotide may include one or more nuclease target site(s), for example, nuclease target sites flanking the transgene to be integrated into the cell's genome. See, e.g., U.S. Patent Publication No. 20130326645.

In some embodiments, the double-stranded template polynucleotide includes sequences (also referred to as transgene) greater than 1 kb in length, for example between 2 and 200 kb, between 2 and 10 kb (or any value therebetween). The double-stranded template polynucleotide also includes at least one nuclease target site, for example. Typically, the nuclease target sites are outside the transgene sequences, for example, 5′ and/or 3′ to the transgene sequences, for cleavage of the transgene. The nuclease cleavage site(s) may be for any nuclease(s). In some embodiments, the nuclease target site(s) contained in the double-stranded template polynucleotide are for the same nuclease(s) used to cleave the endogenous target into which the cleaved template polynucleotide is integrated via homology-independent methods.

In some embodiments, the nucleic acid template system is double stranded. In some embodiments, the nucleic acid template system is single stranded. In some embodiments, the nucleic acid template system comprises a single stranded portion and a double stranded portion.

In some embodiments, the presence of a genetic disruption (e.g., a DNA break, such as described in Section I.B, and a template polynucleotide containing one or more homology arms (e.g., containing nucleic acid sequences homologous sequences surrounding the genetic disruption) can induce or direct HDR, with homologous sequences acting as a template for DNA repair. Based on homology between the endogenous gene sequence surrounding the genetic disruption and the 5′ and/or 3′ homology arms included in the template polynucleotide, cellular DNA repair machinery can use the template polynucleotide to repair the DNA break and resynthesize genetic information at the site of the genetic disruption, thereby effectively inserting or integrating the transgene sequences in the template polynucleotide at or near the site of the genetic disruption. In some embodiments, the genetic disruption can be generated by any of the methods for generating a targeted genetic disruption described herein.

Also provided are polynucleotides (in some aspects, referred to as “template polynucleotides”, e.g., comprising transgene sequences encoding a recombinant CAR or NK cell inhibitory moiety), as described herein. In some embodiments, the provided polynucleotides can be employed in the methods described herein, e.g., involving HDR, to target transgene sequences.

In some embodiments, the template polynucleotide is or comprises a polynucleotide containing a transgene (exogenous or heterologous nucleic acids sequences) encoding a recombinant CAR or an NK cell inhibitory moiety (e.g., a recombinant HLA-E fusion protein or a portion thereof), and homology sequences (e.g., homology arms) that are homologous to sequences at or near the endogenous genomic site, e.g., at the endogenous TRAC locus or at the endogenous B2M locus. In some aspects, the template polynucleotide is introduced as a linear DNA fragment or comprised in a vector. In some aspects, the step for inducing genetic disruption and the step for targeted integration (e.g., by introduction of the template polynucleotide) are performed simultaneously or sequentially.

### 1. CAR Transgene

In some embodiments, the T cell is engineered with a chimeric antigen receptor. Any of a variety of CARs can be engineered into the T cell, such as any described in Section IV. In some embodiments, the CAR is introduced into the T cell by targeted insertion into a genomic loci in the T cell. In some embodiments, the targeted insertion is by HDR. In some embodiments, the targeted insertion is by CRISPR/Cas-mediated HDR of a donor template comprising a polynucleotide sequence encoding the CAR. In some embodiments, the endogenous gene loci is any of the disrupted loci as described herein. In some embodiments, the endogenous gene locus is the endogenous TRAC gene.

In some aspects, in the presence of a genetic disruption at a target site at a TRAC locus (e.g., as described in Section I.B.1), and a polynucleotide, such as the template polynucleotide having homology with sequences at or near the target site in an endogenous TRAC locus, can be used to modify the DNA in the T cell by targeted insertion (for example, a knock-in (KI)) of a transgene (e.g., a recombinant CAR). In some embodiment, the transgene is targeted at or around the TRAC locus, for example by homology-dependent repair (HDR). In some embodiments, the homology sequences of the template polynucleotide target the transgene at a TRAC locus.

In some embodiments, a polynucleotide, such as a template polynucleotide having homology with sequences at or near one or more target site(s) in the endogenous DNA can be used to alter the structure of a target DNA, e.g., targeted insertion of the transgene encoding a recombinant CAR or a portion thereof. In some embodiments, the template polynucleotide contains homology sequences (e.g., homology arms) flanking the transgene, e.g., nucleic acid sequences encoding a recombinant CAR or a portion thereof, for targeted insertion. In some embodiments, the homology sequences target the transgene at a TRAC locus. In some aspects, the transgene encoding the CAR within the template polynucleotide can be used to guide the location of target sites and/or homology arms. In some aspects, the target site of genetic disruption of the TRAC gene can be used as a guide to design template polynucleotides and/or homology arms used for HDR. In some embodiments, the genetic disruption can be targeted near a desired site of targeted integration of transgene sequences (e.g., encoding a recombinant CAR or a portion thereof). In some aspects, the target site is within an exon of the open reading frame of the TRAC locus. In some aspects, the target site is within an intron of the open reading frame of the TRAC locus.

In some embodiments, the template polynucleotide includes additional sequences (coding or non-coding sequences) between the homology arms, such as a regulatory sequences, such as promoters and/or enhancers, splice donor and/or acceptor sites, internal ribosome entry site (IRES), sequences encoding ribosome skipping elements (e.g., 2A peptides), markers and/or SA sites, and/or one or more additional transgenes.

In some embodiments, the transgene contained in the polynucleotide, e.g., template polynucleotide, comprises a sequence encoding a recombinant CAR or a portion thereof. In some embodiments, the transgene can encode any of the recombinant CARs described herein or any chains, regions and/or domains thereof. In some embodiments, the transgene encodes a recombinant chimeric antigen receptor (CAR) or any chains, regions and/or domains thereof. In some aspects, the transgene encodes a CAR comprising a CD19-binding domain. In some aspects, the polynucleotide, e.g., template polynucleotide, comprises any transgene sequences provided herein or a nucleic acid sequence encoding any recombinant CAR described herein, e.g., in Section IV.

In some aspects, the polynucleotide, e.g., template polynucleotide, comprises any transgene sequences provided herein or a nucleic acid sequence encoding any recombinant CAR described herein, e.g., in Section IV. In some embodiments, the encoded recombinant CAR or portion thereof contains one or more domains that shares complete, e.g., at or about 100% identity, to all or a portion and/or fragment of an endogenous CAR constant domain.

In some embodiments, the transgene encoding the CAR comprises the nucleic acid sequence set forth in SEQ ID NO: 94, or a sequence that has at least at or about 90%, 91%, 92%, 93%, 94%, 95%, 96%, 97%, 98%, or 99% sequence identity to SEQ ID NO: 94.

In some embodiments, the transgene comprises a nucleic acid sequence having at or at least 70%, 75%, 80%, 85%, 90%, 95%, 97%, 98%, 99%, 99.5%, or 99.9% sequence identity to all or a portion of the nucleic acid sequence set forth in SEQ ID NO: 94.

In certain embodiments, the polynucleotide, e.g., template polynucleotide contains and/or includes a transgene encoding all or a portion of a recombinant CAR. In particular embodiments, the transgene is targeted at a target site(s) that is within a gene, locus, or open reading frame that encodes an endogenous receptor, e.g., an endogenous gene encoding one or more regions of a CAR.

In some embodiments, the template polynucleotide contains the transgene, e.g., recombinant CAR-encoding nucleic acid sequences, flanked by homology sequences (also called “homology arms”) on the 5′ and 3′ ends, to allow the DNA repair machinery, e.g., homologous recombination machinery, to use the template polynucleotide as a template for repair, effectively inserting the transgene into the target site of integration in the genome. The homology arm should extend at least as far as the region in which end may occur, e.g., in order to allow the resected single stranded overhang to find a complementary region within the template polynucleotide. The overall length could be limited by parameters such as plasmid size or viral packaging limits. In some embodiments, a homology arm does not extend into repeated elements, e.g., ALU repeats or LINE repeats.

Exemplary homology arm lengths include at least or at least about or is or is about 50, 100, 200, 250, 300, 400, 500, 600, 700, 750, 800, 900, 1000, 2000, 3000, 4000, or 5000 nucleotides. In some embodiments, the homology arm length is 50-100, 100-250, 250-500, 500-750, 750-1000, 1000-2000, 2000-3000, 3000-4000, or 4000-5000 nucleotides. Exemplary homology arm lengths include less than or less than about or is or is about 50, 100, 200, 250, 300, 400, 500, 600, 700, 750, 800, 900, 1000, 2000, 3000, 4000, or 5000 nucleotides. In some embodiments, the homology arm length is 50-100, 100-250, 250-500, 500-750, 750-1000, 1000-2000, 2000-3000, 3000-4000, or 4000-5000 nucleotides.

In some embodiments, the template polynucleotide comprises about 500 to 1000, e.g., 600 to 900 or 700 to 800, base pairs of homology on either side of the target site at the endogenous gene, such as a second target site at an endogenous TRAC locus. In some embodiments, the template polynucleotide comprises at least or less than or about 200, 300, 400, 500, 600, 700, 800, 900 or 1000 base pairs, homology 5′ of the target site, 3′ of the target site, or both 5′ and 3′ of the target site, e.g., within the TRAC gene, locus, or open reading frame (e.g., described in Table 1 herein).

In some embodiments, the template polynucleotide comprises about 10, 20, 30, 40, 50, 100, 200, 300, 400, 500, 600, 700, 800, 900, 1000, 1500, 2000, 3000, 4000, or 5000 base pairs homology 3′ of the target site. In some embodiments, the template polynucleotide comprises about 100 to 500, 200 to 400 or 250 to 350, base pairs homology 3′ of the transgene and/or target site. In some embodiments, the template polynucleotide comprises less than about 100, 90, 80, 70, 60, 50, 40, 30, 20, 15, or 10 base pairs homology 5′ of the target site, e.g., within the TRAC gene, locus, or open reading frame (e.g., described in Table 1 herein). In some embodiments, the template polynucleotide comprises about 10, 20, 30, 40, 50, 100, 200, 300, 400, 500, 600, 700, 800, 900, 1000, 1500, 2000, 3000, 4000, or 5000 base pairs homology 5′ of the target site. In some embodiments, the template polynucleotide comprises about 100 to 500, 200 to 400 or 250 to 350, base pairs homology 5′ of the transgene and/or target site. In some embodiments, the template polynucleotide comprises less than about 100, 90, 80, 70, 60, 50, 40, 30, 20, 15, or 10 base pairs homology 3′ of the target site, e.g., within the TRAC gene, locus, or open reading frame (e.g., described in Table 1 herein).

In some embodiments, a template polynucleotide is to a nucleic acid sequence which can be used in conjunction with one or more agent(s) capable of introducing a genetic disruption to alter the structure of a target site. In some embodiments, the target site is modified to have the some or all of the sequence of the template polynucleotide, typically at or near cleavage site(s). In some embodiments, the template polynucleotide is single stranded. In some embodiments, the template polynucleotide is double stranded. In some embodiments, the template polynucleotide is DNA, e.g., double stranded DNA In some embodiments, the template polynucleotide is single stranded DNA. In some embodiments, the template polynucleotide is encoded on the same vector backbone, e.g. AAV genome, plasmid DNA, as the Cas9 and gRNA. In some embodiments, the template polynucleotide is excised from a vector backbone in vivo, e.g., it is flanked by gRNA recognition sequences. In some embodiments, the template polynucleotide is on a separate polynucleotide molecule as the Cas9 and gRNA. In some embodiments, the Cas9 and the gRNA are introduced in the form of a ribonucleoprotein (RNP) complex, and the template polynucleotide is introduced as a polynucleotide molecule, e.g., in a vector.

In some embodiments, the polynucleotide, e.g., template polynucleotide, alters the structure of the target site, e.g., insertion of transgene, by participating in a homology directed repair event. In some embodiments, the template polynucleotide alters the sequence of the target site. In some embodiments, the template polynucleotide includes sequence that corresponds to a site on the target sequence that is cleaved by one or more agent(s) capable of introducing a genetic disruption. In some embodiments, the template polynucleotide includes sequence that corresponds to both, a first site on the target sequence that is cleaved in a first agent capable of introducing a genetic disruption, and a second site on the target sequence that is cleaved in a second agent capable of introducing a genetic disruption.

In some embodiments, a template polynucleotide comprises the following components: [5′ homology arm]-[transgene]-[3′ homology arm]. The homology arms provide for recombination into the chromosome, thus insertion of the transgene into the DNA at or near the cleavage site, e.g., target site(s). In some embodiments, the homology arms flank the most distal target site(s).

In some embodiments, the 3′ end of the 5′ homology arm is the position next to the 5′ end of the transgene. In some embodiments, the 5′ homology arm can extend at least 10, 20, 30, 40, 50, 100, 200, 300, 400, 500, 600, 700, 800, 900, 1000, 1500, 2000, 3000, 4000, or 5000 nucleotides 5′ from the 5′ end of the transgene. In some embodiments, the 5′ end of the 3′ homology arm is the position next to the 3′ end of the transgene. In some embodiments, the 3′ homology arm can extend at least 10, 20, 30, 40, 50, 100, 200, 300, 400, 500, 600, 700, 800, 900, 1000, 1500, 2000, 3000, 4000, or 5000 nucleotides 3′ from the 3′ end of the transgene.

In some embodiments, for targeted insertion, the homology arms, e.g., the 5′ and 3′ homology arms, may each comprise about 1000 base pairs (bp) of sequence flanking the most distal gRNAs (e.g., 1000 bp of sequence on either side of the target site).

It is contemplated herein that one or both homology arms may be shortened to avoid including certain sequence repeat elements, e.g., Alu repeats or LINE elements. For example, a 5′ homology arm may be shortened to avoid a sequence repeat element. In some embodiments, a 3′ homology arm may be shortened to avoid a sequence repeat element. In some embodiments, both the 5′ and the 3′ homology arms may be shortened to avoid including certain sequence repeat elements. It is contemplated herein that template polynucleotides for targeted insertion may be designed for use as a single-stranded oligonucleotide, e.g., a single-stranded oligodeoxynucleotide (ssODN). When using a ssODN, 5′ and 3′ homology arms may range up to about 200 base pairs (bp) in length, e.g., at least 25, 50, 75, 100, 125, 150, 175, or 200 bp in length. Longer homology arms are also contemplated for ssODNs as improvements in oligonucleotide synthesis continue to be made. In some embodiments, a longer homology arm is made by a method other than chemical synthesis, e.g., by denaturing a long double stranded nucleic acid and purifying one of the strands, e.g., by affinity for a strand-specific sequence anchored to a solid substrate.

Similarly, in some embodiments, the template polynucleotide has a 5′ homology arm, a transgene, and a 3′ homology arm, such that the template polynucleotide extends substantially the same distance on either side of the target site. For example, the homology arms may have different lengths, but the transgene may be selected to compensate for this. For example, the transgene may extend further 5′ from the target site than it does 3′ of the target site, but the homology arm 5′ of the target site is shorter than the homology arm 3′ of the target site, to compensate. The converse is also possible, e.g., that the transgene may extend further 3′ from the target site than it does 5′ of the target site, but the homology arm 3′ of the target site is shorter than the homology arm 5′ of the target site, to compensate.

The template polynucleotide can be linear single stranded DNA. In some embodiments, the template polynucleotide is (i) linear single stranded DNA that can anneal to the nicked strand of the target DNA, (ii) linear single stranded DNA that can anneal to the intact strand of the target DNA, (iii) linear single stranded DNA that can anneal to the transcribed strand of the target DNA, (iv) linear single stranded DNA that can anneal to the non-transcribed strand of the target DNA, or more than one of the preceding.

In some embodiments, the template polynucleotide is a single stranded nucleic acid. In another embodiment, the template polynucleotide is a double stranded nucleic acid. In some embodiments, the template polynucleotide is linear double stranded DNA. The length may be, e.g., about 200-5000 nucleotides, e.g., about 200, 300, 400, 500, 600, 700, 800, 900, 1000, 1200, 1400, 1600, 1800, 2000, 2500, 3000, 4000 or 5000 nucleotides. The length may be, e.g., at least 200, 300, 400, 500, 600, 700, 800, 900, 1000, 1200, 1400, 1600, 1800, 2000, 2500, 3000, 4000 or 5000 nucleotides. In some embodiments, the length is no greater than 200, 300, 400, 500, 600, 700, 800, 900, 1000, 1200, 1400, 1600, 1800, 2000, 2500, 3000, 4000 or 5000 nucleotides. In some embodiments, a double stranded template polynucleotide has a length of about 160 nucleotides, e.g., about 200-4000, 300-3500, 400-3000, 500-2500, 600-2000, 700-1900, 800-1800, 900-1700, 1000-1600, 1100-1500 or 1200-1400 nucleotides.

In some embodiments, the template polynucleotide is circular double stranded DNA, e.g., a plasmid. In some embodiments, the template polynucleotide comprises about 500 to 1000 nucleotides of homology on either side of the transgene and/or the target site. In some embodiments, the template polynucleotide comprises about 10, 20, 30, 40, 50, 100, 200, 300, 400, 500, 600, 700, 800, 900, 1000, 1500, or 2000 nucleotides of homology 5′ of the target site or transgene, 3′ of the target site or transgene, or both 5′ and 3′ of the target site or transgene. In some embodiments, the template polynucleotide comprises at least 10, 20, 30, 40, 50, 100, 200, 300, 400, 500, 600, 700, 800, 900, 1000, 1500, or 2000 nucleotides of homology 5′ of the target site or transgene, 3′ of the target site or transgene, or both 5′ and 3′ of the target site or transgene. In some embodiments, the template polynucleotide comprises no more than 10, 20, 30, 40, 50, 100, 200, 300, 400, 500, 600, 700, 800, 900, 1000, 1500, or 2000 nucleotides of homology 5′ of the target site or transgene, 3′ of the target site or transgene, or both 5′ and 3′ of the target site or transgene.

In some embodiments, the template polynucleotide contains homology arms for targeting the endogenous TRAC locus. In some embodiments, the genetic disruption of the TRAC locus is introduced at early coding region the gene, including sequence immediately following a transcription start site, within a first exon of the coding sequence, or within 500 bp of the transcription start site (e.g., less than 500, 450, 400, 350, 300, 250, 200, 150, 100 or 50 bp), or within 500 bp of the start codon (e.g., less than 500, 450, 400, 350, 300, 250, 200, 150, 100 or 50 bp). In some embodiments, the genetic disruption is introduced using any of the targeted nucleases and/or gRNAs described in Section I.B.1 herein. In some embodiments, the template polynucleotide comprises about 500 to 1000, e.g., 600 to 900 or 700 to 800, nucleotides of homology on either side of the genetic disruption introduced by the targeted nucleases and/or gRNAs. In some embodiments, the template polynucleotide comprises about 500, 600, 700, 800, 900 or 1000 nucleotides of 5′ homology arm sequences, which is homologous to 500, 600, 700, 800, 900 or 1000 nucleotides of sequences 5′ of the genetic disruption (e.g., at TRAC locus), the transgene, and about 500, 600, 700, 800, 900 or 1000 nucleotides of 3′ homology arm sequences, which is homologous to 500, 600, 700, 800, 900 or 1000 nucleotides of sequences 3′ of the genetic disruption (e.g., at TRAC locus). In some embodiments, exemplary 5′ and 3′ homology arms for targeted integration at the TRAC locus are set forth in SEQ ID NO: 76 and 77, respectively.

In some instances, the template polynucleotide comprises a promoter, e.g., a promoter that is exogenous and/or not present at or near the target locus. In some embodiments in which the functional polypeptide encoding sequences are promoterless, expression of the integrated transgene is then ensured by transcription driven by an endogenous promoter or other control element in the region of interest.

The transgene, including the transgene encoding the recombinant CAR or a portion thereof, can be inserted so that its expression is driven by the endogenous promoter at the integration site, namely the promoter that drives expression of the endogenous gene into which the transgene is inserted (e.g., TRAC). For example, the coding sequences in the transgene can be inserted without a promoter, but in-frame with the coding sequence of the endogenous target gene, such that expression of the integrated transgene is controlled by the transcription of the endogenous promoter at the integration site. In some embodiments, the transgene encoding the recombinant CAR or a portion thereof and/or the one or more further transgene independently is operably linked to the endogenous promoter of the gene at the target site. In some embodiments, a ribosome skipping element/self-cleavage element, such as a 2A element, is placed upstream of the transgene coding sequence, such that the ribosome skipping element/self-cleavage element is placed in-frame with the endogenous gene, such that the expression of the transgene encoding the recombinant or a portion thereof and/or the one or more further transgene is operably linked to the endogenous promoter.

In some embodiments, the transgene encoding the recombinant CAR or a portion thereof and/or the one or more second transgene independently comprises one or more multicistronic element(s). In some embodiments, the one or more multicistronic element(s) are upstream of the transgene encoding the recombinant CAR or a portion thereof and/or the one or more second transgene. In some embodiments, the multicistronic element(s) is positioned between the transgene encoding the recombinant CAR or a portion thereof and the one or more second transgene. In some embodiments, the ribosome skip element comprises a sequence encoding a ribosome skip element selected from among a T2A, a P2A, a E2A or a F2A or an internal ribosome entry site (IRES).

The transgene may be inserted into an endogenous gene such that all, some or none of the endogenous gene is expressed. In some embodiments, the transgene (e.g., with or without peptide-encoding sequences) is integrated into any endogenous locus. In some embodiments, the transgene is integrated into an endogenous TRAC locus.

In some embodiments, exogenous sequences may also include transcriptional or translational regulatory sequences, for example, promoters, enhancers, insulators, internal ribosome entry sites, sequences encoding 2A peptides and/or polyadenylation signals. Further, the control elements of the genes of interest can be operably linked to reporter genes to create chimeric genes (e.g., reporter expression cassettes). In an exemplary embodiment, the template polynucleotide includes homology arms for targeting at the TRAC locus, regulatory sequences, e.g., promoter, and nucleic acid sequences encoding a recombinant CAR.

In some cases, the ribosome skipping element/self-cleavage element, such as a T2A, can cause the ribosome to skip (ribosome skipping) synthesis of a peptide bond at the C-terminus of a 2A element, leading to separation between the end of the 2A sequence and the next peptide downstream (see, for example, de Felipe, Genetic Vaccines and Ther. 2:13 (2004) and de Felipe et al. Traffic 5:616-626 (2004)). This allows the inserted transgene to be controlled by the transcription of the endogenous promoter at the integration site, e.g., TRAC promoter. Exemplary ribosome skipping element/self-cleavage element include 2A sequences from the foot-and-mouth disease virus (F2A, e.g., SEQ ID NO: 95), equine rhinitis A virus (E2A, e.g., SEQ ID NO: 96), Thosea asigna virus (T2A, e.g., SEQ ID NO: 97 or SEQ ID NO: 98), and porcine teschovirus-1 (P2A, e.g., ID NO: 99 or SEQ ID NO: 100) as described in U.S. Patent Publication No. 20070116690. In some embodiments, the template polynucleotide includes a P2A ribosome skipping element (sequence set forth in SEQ ID NO: 99 or SEQ ID NO: 100 upstream of the transgene, e.g., recombinant TCR encoding nucleic acids or between the sequences encoding a TCRα chain and the sequences encoding a TCRβ chain.

In some embodiments, transgene may comprise a promoter and/or enhancer, for example a constitutive promoter or an inducible or tissue-specific promoter. In some embodiments, the promoter is or comprises a constitutive promoter. Exemplary constitutive promoters include, any described herein, such as a human elongation factor 1a promoter (EF1a). In some embodiments, the constitutive promoter is a synthetic or modified promoter. In some embodiments, the promoter is a tissue-specific promoter or a viral promoter. In some embodiments, the promoter is a non-viral promoter. In some embodiments, the promoter is a modified EF1α promoter with HTLV1 enhancer, for example set forth in SEQ ID NO: 91. In some embodiments, the transgene does not include a regulatory element, e.g. promoter.

In some embodiments, a “tandem” cassette is integrated into the selected site. In some embodiments, one or more of the “tandem” cassettes encode one or more polypeptide or factors, each independently controlled by a regulatory element or all controlled as a multi-cistronic expression system. In some embodiments, such as those where the polynucleotide contains a first and second nucleic acid sequence, the coding sequences encoding each of the different polypeptide chains can be operatively linked to a promoter, which can be the same or different. In some embodiments, the nucleic acid molecule can contain a promoter that drives the expression of two or more different polypeptide chains. In some embodiments, such nucleic acid molecules can be multicistronic (bicistronic or tricistronic, see e.g., U.S. Pat. No. 6,060,273). In some embodiments, transcription units can be engineered as a bicistronic unit containing an IRES (internal ribosome entry site), which allows coexpression of gene products by a message from a single promoter. Alternatively, in some cases, a single promoter may direct expression of an RNA that contains, in a single open reading frame (ORF), two or three polypeptides separated from one another by sequences encoding a self-cleavage peptide (e.g., 2A sequences) or a protease recognition site (e.g., furin), as described herein. The ORF thus encodes a single polypeptide, which, either during (in the case of 2A) or after translation, is processed into the individual proteins. In some embodiments, the “tandem cassette” includes the first component of the cassette comprising a promoterless sequence, followed by a transcription termination sequence, and a second sequence, encoding an autonomous expression cassette or a multi-cistronic expression sequence. In some embodiments, the tandem cassette encodes two or more different polypeptides or factors, e.g., two or more chains or domains of a recombinant TCR. In some embodiments, nucleic acid sequences encoding two or more chains or domains of the recombinant TCR are introduced as tandem expression cassettes or bi- or multi-cistronic cassettes, into one target DNA integration site.

The transgene may be inserted into an endogenous gene such that all, some or none of the endogenous gene is expressed. In some embodiments, the transgene (e.g., with or without peptide-encoding sequences) is integrated into any endogenous locus. In some embodiments, the transgene is integrated into an endogenous TRAC locus.

In some embodiments, exogenous sequences may also include transcriptional or translational regulatory sequences, for example, promoters, enhancers, insulators, internal ribosome entry sites, sequences encoding 2A peptides and/or polyadenylation signals. Further, the control elements of the genes of interest can be operably linked to reporter genes to create chimeric genes (e.g., reporter expression cassettes). In an exemplary embodiment, the template polynucleotide includes homology arms for targeting at the TRAC locus, regulatory sequences, e.g., promoter, and nucleic acid sequences encoding a recombinant TCR.

In some embodiments, exemplary template polynucleotides contain transgene encoding a recombinant T cell receptor under the operable control of the human elongation factor 1 alpha (EF1α) promoter with HTLV1 enhancer (sequence set forth in SEQ ID NO: 91), 5′ homology arm sequence of approximately 600 bp (e.g., set forth in SEQ ID NO: 76), 3′ homology arm sequence of approximately 600 bp (e.g., set forth in SEQ ID NO: 77) that are homologous to sequences surrounding the target integration site in exon 1 of the human TCRα constant domain (TRAC) gene.

In some embodiments, exemplary template polynucleotides contain transgene encoding a CAR (sequence set forth in SEQ ID NO: 136), 5′ homology arm sequence of approximately 600 bp (e.g., set forth in SEQ ID NO: 76), 3′ homology arm sequence of approximately 600 bp (e.g., set forth in SEQ ID NO: 77) that are homologous to sequences surrounding the target integration site in exon 1 of the human TCRα constant domain (TRAC) gene. In some embodiments, the template polynucleotide further contains other nucleic acid sequences, e.g., nucleic acid sequences encoding a marker, e.g., a surface marker or a selection marker. In some embodiments, the template polynucleotide further contains viral vector sequences, e.g., adeno-associated virus (AAV) vector sequences.

A polynucleotide can be introduced into a cell as part of a vector molecule having additional sequences such as, for example, replication origins, promoters and genes encoding antibiotic resistance. Moreover, template polynucleotides can be introduced as naked nucleic acid, as nucleic acid complexed with materials such as a liposome, nanoparticle or poloxamer, or can be delivered by viruses (e.g., adenovirus, AAV, herpesvirus, retrovirus, lentivirus and integrase defective lentivirus (IDLV)).

In other aspects, the template polynucleotide is delivered by viral and/or non-viral gene transfer methods. In some embodiments, the template polynucleotide is delivered to the cell via an adeno associated virus (AAV), such as any described herein.

In some embodiments, the template polynucleotide is comprised in a viral vector, and is at least at or about 2500, 2750, 3000, 3250, 3500, 3750, 4000, 4250, 4500, 4760, 5000, 5250, 5500, 5750, 6000, 7000, 7500, 8000, 9000 or 10000 nucleotides in length, or any value between any of the foregoing. In some embodiments, the polynucleotide is comprised in a viral vector, and is between at or about 2500 and at or about 5000 nucleotides, at or about 3500 and at or about 4500 nucleotides, or at or about 3750 nucleotides and at or about 4250 nucleotides in length. In some embodiments, the polynucleotide is comprised in a viral vector, and is at or about 2500, 2750, 3000, 3250, 3500, 3750, 4000, 4250, 4500, 4760, 5000, 5250, 5500, 5750, 6000, 7000, 7500, 8000, 9000 or 10000 nucleotides in length.

In some embodiments, the template polynucleotide is an adenovirus vector, e.g., an AAV vector, e.g., a ssDNA molecule of a length and sequence that allows it to be packaged in an AAV capsid. The vector may be, e.g., less than 5 kb and may contain an ITR sequence that promotes packaging into the capsid. The vector may be integration-deficient. In some embodiments, the template polynucleotide comprises about 150 to 1000 nucleotides of homology on either side of the transgene and/or the target site. In some embodiments, the template polynucleotide comprises about 100, 150, 200, 300, 400, 500, 600, 700, 800, 900, 1000, 1500, or 2000 nucleotides 5′ of the target site or transgene, 3′ of the target site or transgene, or both 5′ and 3′ of the target site or transgene. In some embodiments, the template polynucleotide comprises at least 100, 150, 200, 300, 400, 500, 600, 700, 800, 900, 1000, 1500, or 2000 nucleotides 5′ of the target site or transgene, 3′ of the target site or transgene, or both 5′ and 3′ of the target site or transgene. In some embodiments, the template polynucleotide comprises at most 100, 150, 200, 300, 400, 500, 600, 700, 800, 900, 1000, 1500, or 2000 nucleotides 5′ of the target site or transgene, 3′ of the target site or transgene, or both 5′ and 3′ of the target site or transgene.

In some embodiments, the template polynucleotide is a lentiviral vector, e.g., an IDLV (integration deficiency lentivirus).

The double-stranded template polynucleotides described herein may include one or more non-natural bases and/or backbones. In particular, insertion of a template polynucleotide with methylated cytosines may be carried out using the methods described herein to achieve a state of transcriptional quiescence in a region of interest.

The polynucleotide may comprise any transgene of interest (exogenous sequence). Exemplary exogenous sequences include, but are not limited to any polypeptide coding sequence (e.g., cDNAs or fragments thereof), promoter sequences, enhancer sequences, epitope tags, marker genes, cleavage enzyme recognition sites and various types of expression constructs. Marker genes include, but are not limited to, sequences encoding proteins that mediate antibiotic resistance (e.g., ampicillin resistance, neomycin resistance, G418 resistance, puromycin resistance), sequences encoding colored or fluorescent or luminescent proteins (e.g., green fluorescent protein, enhanced green fluorescent protein, red fluorescent protein, luciferase), and proteins which mediate enhanced cell growth and/or gene amplification (e.g., dihydrofolate reductase). Epitope tags include, for example, one or more copies of FLAG, His, myc, Tap, HA or any detectable amino acid sequence.

In some embodiments, the transgene comprises a polynucleotide encoding any polypeptide of which expression in the cell is desired, including, but not limited to antibodies, antigens, enzymes, receptors (cell surface or nuclear), hormones, lymphokines, cytokines, reporter polypeptides, growth factors, and functional fragments of any of the foregoing. In some embodiments, the coding sequences may be, for example, cDNAs.

In some embodiments, the transgene further encodes one or more marker(s). In some embodiments, the one or more marker(s) is a transduction marker, surrogate marker and/or a selection marker.

In some embodiments, the marker is a transduction marker or a surrogate marker. A transduction marker or a surrogate marker can be used to detect cells that have been introduced with the polynucleotide, e.g., a polynucleotide encoding a recombinant CAR. In some embodiments, the transduction marker can indicate or confirm modification of a cell. In some embodiments, the surrogate marker is a protein that is made to be co-expressed on the cell surface with the recombinant CAR. In particular embodiments, such a surrogate marker is a surface protein that has been modified to have little or no activity. In certain embodiments, the surrogate marker is encoded on the same polynucleotide that encodes the recombinant TCR. In some embodiments, the nucleic acid sequence encoding the recombinant TCR is operably linked to a nucleic acid sequence encoding a marker, optionally separated by an internal ribosome entry site (IRES), or a nucleic acid encoding a self-cleaving peptide or a peptide that causes ribosome skipping, such as a 2A sequence, such as a T2A, a P2A, an E2A or an F2A. Extrinsic marker genes may in some cases be utilized in connection with engineered cell to permit detection or selection of cells and, in some cases, also to promote cell suicide.

In some embodiments, the marker is a molecule, e.g., cell surface protein, not naturally found on T cells or not naturally found on the surface of T cells, or a portion thereof.

In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

In some embodiments, the polynucleotide contains the structure: [5′ homology arm]-[transgene sequence]-[3′ homology arm]. In some embodiments, the polynucleotide contains the structure: [5′ homology arm]-[multicistronic element]-[transgene sequence]-[3′ homology arm]. In some embodiments, the polynucleotide contains the structure: [5′ homology arm]-[promoter]-[transgene sequence]-[3′ homology arm].

Construction of such expression cassettes, following the teachings of the present specification, utilizes methodologies well known in molecular biology (see, for example, Ausubel or Maniatis). Before use of the expression cassette to generate a transgenic animal, the responsiveness of the expression cassette to the stress-inducer associated with selected control elements can be tested by introducing the expression cassette into a suitable cell line (e.g., primary cells, transformed cells, or immortalized cell lines).

### 2. NK Cell Inhibitory Moiety

In some embodiments, the T cell is genetically engineered with an NK cell inhibitory moiety that is a recombinant ligand of an NK inhibitory receptor. In some aspects, the provided engineered cells lack endogenous expression of, or have reduced expression of, a ligand for an NK inhibitory receptor, which may otherwise render the cell susceptible to NK cell-mediated cytotoxicity. For instance, as a result of complete elimination of B2M in accord with provided methods, T cells can become more vulnerable to attack by Natural Killer (NK) cells, which treat them as non-self.

In some embodiments, the NK cell inhibitory moiety is introduced into the T cell by targeted insertion into a genomic loci in the T cell. In some embodiments, the targeted insertion is by HDR. In some embodiments, the targeted insertion is by CRISPR/Cas-mediated HDR of a donor template comprising a polynucleotide sequence encoding the NK cell inhibitory moiety. In some embodiments, the endogenous gene loci is any of the disrupted loci as described herein. In some embodiments, the endogenous gene locus is the endogenous B2M gene.

In some aspects, in the presence of a genetic disruption at a target site at a B2M locus (e.g., as described in Section I.B.2), and a polynucleotide, such as the template polynucleotide having homology with sequences at or near the target site in an endogenous B2M locus, can be used to modify the DNA in the T cell by targeted insertion (for example, a knock-in (KI)) of a transgene (e.g., encoding a recombinant HLA-E fusion protein). In some embodiments, the targeted insertion is at or around the B2M locus, for example by homology-dependent repair (HDR). In some embodiments, the homology sequences of the template polynucleotide target the transgene at a B2M locus.

In some aspects, the transgene (e.g., exogenous nucleic acid sequences) within the template polynucleotide can be used to guide the location of target sites and/or homology arms. In some aspects, the target site of genetic disruption can be used as a guide to design template polynucleotides and/or homology arms used for HDR. In some embodiments, the genetic disruption can be targeted near a desired site of targeted integration of transgene sequences (e.g., encoding a recombinant HLA-E fusion protein or a portion thereof). In some aspects, the target site is within an exon of the open reading frame of the B2M locus. In some aspects, the target site is within an intron of the open reading frame of the B2M locus.

In some embodiments, the recombinant NK cell modulator includes a ligand or binding portion of a ligand capable of binding to an NK cell inhibitory receptor CD94/NKG2A, LIR-1/ILT2, KIR2DL4, LIR-2/ILT4 or SIRPα.

In some embodiments, the NK cell inhibitory moiety is an MHC-E (or HLA-E), MHC-G (or HLA-G) or CD47.

In some embodiments, the NK cell inhibitory moiety is CD47 (e.g., NCBI Ref. Sequence Nos. NP_001768.1 and NP_942088.1). CD47 engages with SIRPα and thrombospondin-1 (TSP-1) and has established its role as an inhibitory receptor involved in immune evasion by cancers through inhibition of phagocytosis, antigen presentation, and T/NK cell inhibition. In particular, engagement of SIRPα by CD47 mediates a strong inhibitory signal in NK cells. An exemplary sequence of CD47 is set forth in SEQ ID NO: 135. In some embodiments, the cell comprises a CD47 polypeptide having at least 95%, 96%, 97%, 98%, 99%, or more sequence identity to an amino acid sequence as set forth in SEQ ID NO: 135. In some embodiments, the cell comprises a CD47 polypeptide having the amino acid sequence as set forth in SEQ ID NO: 135. In some embodiments, the polynucleotide encoding CD47 is operably linked to a promoter. In some embodiments, a transgene polynucleotide sequence encoding CD47 is integrated into the genome of the cell by targeted or non-targeted methods of insertion, such as described further below.

In some embodiments, the NK cell inhibitory moiety includes an HLA-E or an HLA-G molecule. The sequence of an exemplary HLA-E is set forth in SEQ ID NO: 132 (corresponding to amino acids 164 to 500 of SEQ ID NO: 81). The sequence of an exemplary HLA-G is set forth in SEQ ID NO: 133. Expression of HLA-E and HLA-G on the surface of cells can be recognized by inhibitory receptor on NK cells to modulate NK cell activation. In some cases, the binding of peptides (e.g. nonameric peptides), typically derived from signal peptides of classical MHC class I molecules, can stabilize expression of HLA-E and HLA-G on the surface of cells. Typically, like the classical MHC molecules, HLA-E and HLA-G are expressed as a heterodimer containing an α heavy chain and a light chain (also called β-2 microglobulin). Thus, stable expression of HLA-E and HLA-G NK cell inhibitory receptors typically also require expression of B2M. In some cases, the complex of NKG2A and CD94 is involved in the recognition of HLA-E and its peptide (e.g. derived from a leader sequence of another peptide), which can mediate an inhibitory signal by the NK cell. In some embodiments, the inhibitory signal of HLA-G is mediated through the interaction with the NK receptors LIR-1/ILT2, KIR2DL4 and, in some cases, ILT4.

In some embodiments, an HLA-E chain or HLA-G chain can be introduced into the cells, such as by using an expression vector. In some embodiments, the cell also expresses a β2 microglobulin (β2M) or a component or functional fragment thereof. In some cases, at least a portion of the β2M enhances proper MHC folding and expression on the cell surface, including proper folding and expression of HLA-E or HLA-G. An exemplary sequence of β2M is set forth in SEQ ID NO: 134. In some embodiments, the β2M is covalently associated with the HLA-E or HLA-G. In some embodiments, the β2M is expressed as a hybrid or fusion molecule with HLA-E or HLA-G. In some embodiments, a single HLA-E chain or HLA-G chain and β2-microglobulin can be introduced into cell as a fusion protein. In some embodiments, expression of a recombinant HLA-E or HLA-G molecule on the surface of the cell can be stabilized by the addition of a binding peptide. In some embodiments, the binding peptide comprises a nonameric peptide.

Single chain fusion molecules of MHC proteins are known and described in the art (see e.g. published U.S. Pat. Appl. No. US20050196404. In some embodiments, the single chain fusion HLA-E or HLA-G protein comprises $2M or a functional portion thereof covalently linked to the mature α chain HLA-E or HLA-G or functional portion thereof. In some embodiments, the α chain HLA-E or HLA-G can include a transmembrane domain for cell surface expression of the fusion molecule. In some embodiments, the transmembrane domain is the native transmembrane domain of the α chain of the HLA-E or HLA-G. In some embodiments, the single chain fusion can further include a HLA-E or HLA-G binding peptide sufficient to stabilize expression of the HLA-E or HLA-G molecule on the surface. For example, in some embodiment, for stable expression of HLA-E, the binding peptide is a leader sequence of another MHC class I molecule, such as a classical MHC class I molecule, as described or known in the art.

In some embodiments, the binding peptide is a portion of a signal sequence from an MHC class I molecule. In some embodiments, the binding peptide is VMAPRTLVL (SEQ ID NO: 107), VMAPRTLLL (SEQ ID NO: 108), VMAPRTVLL (SEQ ID NO: 109), VMAPRTLFL (SEQ ID NO: 110), or VMAPRTLIL (SEQ ID NO: 111). In some embodiments, the binding peptide is VMAPRTLVL (SEQ ID NO: 107).

In some embodiments, the single chain MHC fusion molecule includes one or more linkers joining the components of the fusion molecule. In some embodiments, the fusion comprises one or more linkers between the binding peptide and B2M, the B2M and class I (e.g. HLA-E) α chain and/or between the binding peptide and class I α chain. In some embodiments, the fusion molecule is constructed to contain in order: HLA-E binding peptide, linker 1, B2M, linker 2 and HLA-E α chain. The linker typically is a peptide linker, e.g., a flexible and/or soluble peptide linker. Among the linkers are those rich in glycine and serine and/or in some cases threonine. In some embodiments, the linker comprises 10 to 20 residues, such as at least or about 10, 15, or 20 residues. In some embodiments, one or more linkers is (G₄S)₃₋₄ (SEQ ID NO: 101). In some embodiments, the linker is (G₄S)₂₋₃ (SEQ ID NO: 102) or GGGAS(G₄S)₂ (SEQ ID NO: 103). In some embodiments, the encoding nucleic acid molecule of the HLA-E fusion protein can include an N-terminal signal sequence for entry into the ER is required. In some embodiments, the signal sequence of B2M is normally used.

In some embodiments, the MHC molecule is a single chain trimer (SCT). In some embodiments, the SCT comprises a single polypeptide comprising an antigenic peptide followed by a first flexible linker that connects the C terminus of the peptide to the N terminus of a B2M, and a second flexible linker that connects the C terminus of the B2M with the N terminus of a heavy chain of an HLA-E molecule. In some embodiments, the linker comprises a cysteine, which can form a disulfide bond with a cysteine on the HLA-E heavy chain, including a disulfide trap SCT (dtSCT). For example, in some embodiments the linker between the peptide and the B2M comprises the sequence GCGASGGGGSGGGGS (SEQ ID NO: 104). Examples of SCT molecules are known in the art, including, for example, as described in US20050196404.

In some embodiments, the covalently linked peptide epitope is cleaved via a built-in protease cleavage site, and the cleaved peptide epitope can bind to the peptide binding site of the single chain protein for stabilization of the molecule.

In some embodiments, the transgene encoding the HLA-E fusion protein comprises a nucleotide sequence recited in SEQ ID NO: 138 or a sequence that has at least at or about 90%, 91%, 92%, 93%, 94%, 95%, 96%, 97%, 98%, or 99% sequence identity to SEQ ID NO: 138. In some embodiments, the nucleotide sequence of the transgene is recited in SEQ ID NO: 138.

In some embodiments, a polynucleotide, such as a template polynucleotide having homology with sequences at or near one or more target site(s) in the endogenous DNA can be used to alter the structure of a target DNA, e.g., targeted insertion of the transgene encoding an NK cell inhibitory moiety, such as a recombinant HLA-E fusion protein or a portion thereof. In some embodiments, the template polynucleotide contains homology sequences (e.g., homology arms) flanking the transgene, e.g., nucleic acid sequences encoding a recombinant HLA-E fusion protein or a portion thereof, for targeted insertion. In some embodiments, the homology sequences target the transgene at a B2M locus. In some embodiments, the template polynucleotide includes additional sequences (coding or non-coding sequences) between the homology arms, such as a regulatory sequences, such as promoters and/or enhancers, splice donor and/or acceptor sites, internal ribosome entry site (IRES), sequences encoding ribosome skipping elements (e.g., 2A peptides), markers and/or SA sites, and/or one or more additional transgenes. In some embodiments, the transgene contained in the polynucleotide, e.g., template polynucleotide, comprises a sequence encoding a recombinant HLA-E fusion protein or a portion thereof. In some embodiments, the transgene can encode any of the recombinant HLA-E molecules described herein. In some aspects, the polynucleotide, e.g., template polynucleotide, comprises any transgene sequences provided herein or a nucleic acid sequence encoding any recombinant HLA-E described herein.

In certain embodiments, the polynucleotide, e.g., template polynucleotide contains and/or includes a transgene encoding all or a portion of a recombinant HLA-E fusion protein. In particular embodiments, the transgene is targeted at a target site(s) that is within a gene, locus, or open reading frame that encodes an endogenous receptor, e.g., an endogenous gene encoding one or more regions of a HLA-E fusion protein.

In some embodiments, the template polynucleotide contains the transgene, e.g., recombinant HLA-E-encoding nucleic acid sequences, flanked by homology sequences (also called “homology arms”) on the 5′ and 3′ ends, to allow the DNA repair machinery, e.g., homologous recombination machinery, to use the template polynucleotide as a template for repair, effectively inserting the transgene into the target site of integration in the genome. The homology arm should extend at least as far as the region in which end resection may occur, e.g., in order to allow the resected single stranded overhang to find a complementary region within the template polynucleotide. The overall length could be limited by parameters such as plasmid size or viral packaging limits. In some embodiments, a homology arm does not extend into repeated elements, e.g., ALU repeats or LINE repeats.

Exemplary homology arm lengths include at least or at least about or is or is about 50, 100, 200, 250, 300, 400, 500, 600, 700, 750, 800, 900, 1000, 2000, 3000, 4000, or 5000 nucleotides. In some embodiments, the homology arm length is 50-100, 100-250, 250-500, 500-750, 750-1000, 1000-2000, 2000-3000, 3000-4000, or 4000-5000 nucleotides. Exemplary homology arm lengths include less than or less than about or is or is about 50, 100, 200, 250, 300, 400, 500, 600, 700, 750, 800, 900, 1000, 2000, 3000, 4000, or 5000 nucleotides. In some embodiments, the homology arm length is 50-100, 100-250, 250-500, 500-750, 750-1000, 1000-2000, 2000-3000, 3000-4000, or 4000-5000 nucleotides.

In some embodiments, the template polynucleotide comprises about 500 to 1000, e.g., 600 to 900 or 700 to 800, base pairs of homology on either side of the target site at the endogenous gene, such as a second target site at an endogenous B2M locus. In some embodiments, the template polynucleotide comprises at least or less than or about 200, 300, 400, 500, 600, 700, 800, 900 or 1000 base pairs, homology 5′ of the target site, 3′ of the target site, or both 5′ and 3′ of the target site, e.g., within the B2M gene, locus, or open reading frame.

In some embodiments, the template polynucleotide comprises about 10, 20, 30, 40, 50, 100, 200, 300, 400, 500, 600, 700, 800, 900, 1000, 1500, 2000, 3000, 4000, or 5000 base pairs homology 3′ of the target site. In some embodiments, the template polynucleotide comprises about 100 to 500, 200 to 400 or 250 to 350, base pairs homology 3′ of the transgene and/or target site. In some embodiments, the template polynucleotide comprises less than about 100, 90, 80, 70, 60, 50, 40, 30, 20, 15, or 10 base pairs homology 5′ of the target site, e.g., within the B2M gene, locus, or open reading frame. In some embodiments, the template polynucleotide comprises about 10, 20, 30, 40, 50, 100, 200, 300, 400, 500, 600, 700, 800, 900, 1000, 1500, 2000, 3000, 4000, or 5000 base pairs homology 5′ of the target site. In some embodiments, the template polynucleotide comprises about 100 to 500, 200 to 400 or 250 to 350, base pairs homology 5′ of the transgene and/or target site. In some embodiments, the template polynucleotide comprises less than about 100, 90, 80, 70, 60, 50, 40, 30, 20, 15, or 10 base pairs homology 3′ of the target site, e.g., within the B2M gene, locus, or open reading frame.

In some embodiments, a template polynucleotide is to a nucleic acid sequence which can be used in conjunction with one or more agent(s) capable of introducing a genetic disruption to alter the structure of a target site. In some embodiments, the target site is modified to have the some or all of the sequence of the template polynucleotide, typically at or near cleavage site(s). In some embodiments, the template polynucleotide is single stranded. In some embodiments, the template polynucleotide is double stranded. In some embodiments, the template polynucleotide is DNA, e.g., double stranded DNA In some embodiments, the template polynucleotide is single stranded DNA. In some embodiments, the template polynucleotide is encoded on the same vector backbone, e.g. AAV genome, plasmid DNA, as the Cas12a and gRNA. In some embodiments, the template polynucleotide is excised from a vector backbone in vivo, e.g., it is flanked by gRNA recognition sequences. In some embodiments, the template polynucleotide is on a separate polynucleotide molecule as the Cas12a and gRNA. In some embodiments, the Cas12a and the gRNA are introduced in the form of a ribonucleoprotein (RNP) complex, and the template polynucleotide is introduced as a polynucleotide molecule, e.g., in a vector. Types or nucleic acids and vectors for delivery include any of those described in Section I.D.

In some embodiments, the polynucleotide, e.g., template polynucleotide, alters the structure of the target site, e.g., insertion of transgene, by participating in a homology directed repair event. In some embodiments, the template polynucleotide alters the sequence of the target site. In some embodiments, the template polynucleotide includes sequence that corresponds to a site on the target sequence that is cleaved by one or more agent(s) capable of introducing a genetic disruption. In some embodiments, the template polynucleotide includes sequence that corresponds to both, a first site on the target sequence that is cleaved in a first agent capable of introducing a genetic disruption, and a second site on the target sequence that is cleaved in a second agent capable of introducing a genetic disruption.

In some embodiments, a template polynucleotide comprises the following components: [5′ homology arm]-[transgene]-[3′ homology arm]. The homology arms provide for recombination into the chromosome, thus insertion of the transgene into the DNA at or near the cleavage site, e.g., target site(s). In some embodiments, the homology arms flank the most distal target site(s).

In some embodiments, the 3′ end of the 5′ homology arm is the position next to the 5′ end of the transgene. In some embodiments, the 5′ homology arm can extend at least 10, 20, 30, 40, 50, 100, 200, 300, 400, 500, 600, 700, 800, 900, 1000, 1500, 2000, 3000, 4000, or 5000 nucleotides 5′ from the 5′ end of the transgene. In some embodiments, the 5′ end of the 3′ homology arm is the position next to the 3′ end of the transgene. In some embodiments, the 3′ homology arm can extend at least 10, 20, 30, 40, 50, 100, 200, 300, 400, 500, 600, 700, 800, 900, 1000, 1500, 2000, 3000, 4000, or 5000 nucleotides 3′ from the 3′ end of the transgene.

In some embodiments, for targeted insertion, the homology arms, e.g., the 5′ and 3′ homology arms, may each comprise about 1000 base pairs (bp) of sequence flanking the most distal gRNAs (e.g., 1000 bp of sequence on either side of the target site).

It is contemplated herein that one or both homology arms may be shortened to avoid including certain sequence repeat elements, e.g., Alu repeats or LINE elements. For example, a 5′ homology arm may be shortened to avoid a sequence repeat element. In some embodiments, a 3′ homology arm may be shortened to avoid a sequence repeat element. In some embodiments, both the 5′ and the 3′ homology arms may be shortened to avoid including certain sequence repeat elements. It is contemplated herein that template polynucleotides for targeted insertion may be designed for use as a single-stranded oligonucleotide, e.g., a single-stranded oligodeoxynucleotide (ssODN). When using a ssODN, 5′ and 3′ homology arms may range up to about 200 base pairs (bp) in length, e.g., at least 25, 50, 75, 100, 125, 150, 175, or 200 bp in length. Longer homology arms are also contemplated for ssODNs as improvements in oligonucleotide synthesis continue to be made. In some embodiments, a longer homology arm is made by a method other than chemical synthesis, e.g., by denaturing a long double stranded nucleic acid and purifying one of the strands, e.g., by affinity for a strand-specific sequence anchored to a solid substrate.

Similarly, in some embodiments, the template polynucleotide has a 5′ homology arm, a transgene, and a 3′ homology arm, such that the template polynucleotide extends substantially the same distance on either side of the target site. For example, the homology arms may have different lengths, but the transgene may be selected to compensate for this. For example, the transgene may extend further 5′ from the target site than it does 3′ of the target site, but the homology arm 5′ of the target site is shorter than the homology arm 3′ of the target site, to compensate. The converse is also possible, e.g., that the transgene may extend further 3′ from the target site than it does 5′ of the target site, but the homology arm 3′ of the target site is shorter than the homology arm 5′ of the target site, to compensate.

The template polynucleotide can be linear single stranded DNA. In some embodiments, the template polynucleotide is (i) linear single stranded DNA that can anneal to the nicked strand of the target DNA, (ii) linear single stranded DNA that can anneal to the intact strand of the target DNA, (iii) linear single stranded DNA that can anneal to the transcribed strand of the target DNA, (iv) linear single stranded DNA that can anneal to the non-transcribed strand of the target DNA, or more than one of the preceding.

In some embodiments, the template polynucleotide is a single stranded nucleic acid. In another embodiment, the template polynucleotide is a double stranded nucleic acid. In some embodiments, the template polynucleotide is linear double stranded DNA. The length may be, e.g., about 200-5000 nucleotides, e.g., about 200, 300, 400, 500, 600, 700, 800, 900, 1000, 1200, 1400, 1600, 1800, 2000, 2500, 3000, 4000 or 5000 nucleotides. The length may be, e.g., at least 200, 300, 400, 500, 600, 700, 800, 900, 1000, 1200, 1400, 1600, 1800, 2000, 2500, 3000, 4000 or 5000 nucleotides. In some embodiments, the length is no greater than 200, 300, 400, 500, 600, 700, 800, 900, 1000, 1200, 1400, 1600, 1800, 2000, 2500, 3000, 4000 or 5000 nucleotides. In some embodiments, a double stranded template polynucleotide has a length of about 160 nucleotides, e.g., about 200-4000, 300-3500, 400-3000, 500-2500, 600-2000, 700-1900, 800-1800, 900-1700, 1000-1600, 1100-1500 or 1200-1400 nucleotides.

In some embodiments, the template polynucleotide is circular double stranded DNA, e.g., a plasmid. In some embodiments, the template polynucleotide comprises about 500 to 1000 nucleotides of homology on either side of the transgene and/or the target site. In some embodiments, the template polynucleotide comprises about 10, 20, 30, 40, 50, 100, 200, 300, 400, 500, 600, 700, 800, 900, 1000, 1500, or 2000 nucleotides of homology 5′ of the target site or transgene, 3′ of the target site or transgene, or both 5′ and 3′ of the target site or transgene. In some embodiments, the template polynucleotide comprises at least 10, 20, 30, 40, 50, 100, 200, 300, 400, 500, 600, 700, 800, 900, 1000, 1500, or 2000 nucleotides of homology 5′ of the target site or transgene, 3′ of the target site or transgene, or both 5′ and 3′ of the target site or transgene. In some embodiments, the template polynucleotide comprises no more than 10, 20, 30, 40, 50, 100, 200, 300, 400, 500, 600, 700, 800, 900, 1000, 1500, or 2000 nucleotides of homology 5′ of the target site or transgene, 3′ of the target site or transgene, or both 5′ and 3′ of the target site or transgene.

In some embodiments, the length of any of the polynucleotides, e.g., template polynucleotides, is at or about 200-10000 nucleotides, e.g., at or about 200, 300, 400, 500, 600, 700, 800, 900, 1000, 1200, 1400, 1600, 1800, 2000, 2500, 3000, 4000, 5000, 6000, 7000, 8000, 9000 or 10000 nucleotides, or a value between any of the foregoing. In some embodiments, the length is at least at or about 200, 300, 400, 500, 600, 700, 800, 900, 1000, 1200, 1400, 1600, 1800, 2000, 2500, 3000, 4000, 5000, 6000, 7000, 8000, 9000 or 10000 nucleotides, or a value between any of the foregoing. In some embodiments, the length is no greater than at or about 200, 300, 400, 500, 600, 700, 800, 900, 1000, 1200, 1400, 1600, 1800, 2000, 2500, 3000, 4000, 5000, 6000, 7000, 8000, 9000 or 10000 nucleotides. In some embodiments, the length is at or about 200-4000, 300-3500, 400-3000, 500-2500, 600-2000, 700-1900, 800-1800, 900-1700, 1000-1600, 1100-1500 or 1200-1400 nucleotides. In some embodiments, the polynucleotide is at least at or about 2500, 2750, 3000, 3250, 3500, 3750, 4000, 4250, 4500, 4760, 5000, 5250, 5500, 5750, 6000, 7000, 7500, 8000, 9000 or 10000 nucleotides in length, or any value between any of the foregoing. In some embodiments, the polynucleotide is between at or about 2500 and at or about 5000 nucleotides, at or about 3500 and at or about 4500 nucleotides, or at or about 3750 nucleotides and at or about 4250 nucleotides in length. In some embodiments, the polynucleotide is at or about 2500, 2750, 3000, 3250, 3500, 3750, 4000, 4250, 4500, 4760, 5000, 5250, 5500, 5750, 6000, 7000, 7500, 8000, 9000 or 10000 nucleotides in length. The length is about 200-5000 base pairs, e.g., about 200, 300, 400, 500, 600, 700, 800, 900, 1000, 1200, 1400, 1600, 1800, 2000, 2500, 3000, 4000 or 5000 nucleotides. The length is at least 200, 300, 400, 500, 600, 700, 800, 900, 1000, 1200, 1400, 1600, 1800, 2000, 2500, 3000, 4000 or 5000 nucleotides. In some embodiments, the length is no greater than 200, 300, 400, 500, 600, 700, 800, 900, 1000, 1200, 1400, 1600, 1800, 2000, 2500, 3000, 4000 or 5000 nucleotides. In some embodiments, a single stranded template polynucleotide has a length of about 160 nucleotides, e.g., about 200-4000, 300-3500, 400-3000, 500-2500, 600-2000, 700-1900, 800-1800, 900-1700, 1000-1600, 1100-1500 or 1200-1400 nucleotides.

In some embodiments, the template polynucleotide contains homology arms for targeting the endogenous B2M locus. In some embodiments, the genetic disruption of the B2M locus is introduced at early coding region the gene, including sequence immediately following a transcription start site, within a first exon of the coding sequence, or within 500 bp of the transcription start site (e.g., less than 500, 450, 400, 350, 300, 250, 200, 150, 100 or 50 bp), or within 500 bp of the start codon (e.g., less than 500, 450, 400, 350, 300, 250, 200, 150, 100 or 50 bp). In some embodiments, the genetic disruption is introduced using any of the targeted nucleases and/or gRNAs described in Section I.B herein. In some embodiments, the template polynucleotide comprises about 500 to 1000, e.g., 600 to 900 or 700 to 800, nucleotides of homology on either side of the genetic disruption introduced by the targeted nucleases and/or gRNAs. In some embodiments, the template polynucleotide comprises about 500, 600, 700, 800, 900 or 1000 nucleotides of 5′ homology arm sequences, which is homologous to 500, 600, 700, 800, 900 or 1000 nucleotides of sequences 5′ of the genetic disruption (e.g., at B2M locus), the transgene, and about 500, 600, 700, 800, 900 or 1000 nucleotides of 3′ homology arm sequences, which is homologous to 500, 600, 700, 800, 900 or 1000 nucleotides of sequences 3′ of the genetic disruption (e.g., at B2M locus). In some embodiments, exemplary 5′ and 3′ homology arms for targeted integration at the B2M locus are set forth in SEQ ID NO: 79 and SEQ ID NO: 80, respectively.

In some instances, the template polynucleotide comprises a promoter, e.g., a promoter that is exogenous and/or not present at or near the target locus. In some embodiments in which the functional polypeptide encoding sequences are promoterless, expression of the integrated transgene is then ensured by transcription driven by an endogenous promoter or other control element in the region of interest.

The transgene, including the transgene encoding the recombinant HLA-E fusion protein or a portion thereof, can be inserted so that its expression is driven by the endogenous promoter at the integration site, namely the promoter that drives expression of the endogenous gene into which the transgene is inserted (e.g., B2M). For example, the coding sequences in the transgene can be inserted without a promoter, but in-frame with the coding sequence of the endogenous target gene, such that expression of the integrated transgene is controlled by the transcription of the endogenous promoter at the integration site. In some embodiments, the transgene encoding the recombinant HLA-E fusion protein or a portion thereof and/or the one or more further transgene independently is operably linked to the endogenous promoter of the gene at the target site. In some embodiments, a ribosome skipping element/self-cleavage element, such as a 2A element, is placed upstream of the transgene coding sequence, such that the ribosome skipping element/self-cleavage element is placed in-frame with the endogenous gene, such that the expression of the transgene encoding the recombinant or a portion thereof and/or the one or more further transgene is operably linked to the endogenous promoter.

In some embodiments, the transgene encoding the recombinant HLA-E fusion protein or a portion thereof and/or the one or more further transgene independently comprises one or more multicistronic element(s). In some embodiments, the one or more multicistronic element(s) are upstream of the transgene encoding the recombinant HLA-E fusion protein or a portion thereof and/or the one or more second transgene. In some embodiments, the multicistronic element(s) is positioned between the transgene encoding the recombinant HLA-E fusion protein or a portion thereof and the one or more second transgene. In some embodiments, the ribosome skip element comprises a sequence encoding a ribosome skip element selected from among a T2A, a P2A, a E2A or a F2A or an internal ribosome entry site (IRES).

The transgene may be inserted into an endogenous gene such that all, some or none of the endogenous gene is expressed. In some embodiments, the transgene (e.g., with or without peptide-encoding sequences) is integrated into any endogenous locus. In some embodiments, the transgene is integrated into an endogenous B2M locus.

In some embodiments, exogenous sequences may also include transcriptional or translational regulatory sequences, for example, promoters, enhancers, insulators, internal ribosome entry sites, sequences encoding 2A peptides and/or polyadenylation signals. Further, the control elements of the genes of interest can be operably linked to reporter genes to create chimeric genes (e.g., reporter expression cassettes). In an exemplary embodiment, the template polynucleotide includes homology arms for targeting at the B2M locus, regulatory sequences, e.g., promoter, and nucleic acid sequences encoding a recombinant HLA-E.

In some embodiments, exemplary template polynucleotides contain a transgene encoding a HLA-E fusion protein (sequence set forth in SEQ ID NO: 86), 5′ homology arm sequence of approximately 800 bp (e.g., set forth in SEQ ID NO: 79), 3′ homology arm sequence of approximately 800 bp (e.g., set forth in SEQ ID NO: 80) that are homologous to sequences surrounding the target integration site of the human B2M gene. In some embodiments, the template polynucleotide further contains other nucleic acid sequences, e.g., nucleic acid sequences encoding a marker, e.g., a surface marker or a selection marker. In some embodiments, the template polynucleotide further contains viral vector sequences, e.g., adeno-associated virus (AAV) vector sequences. In some embodiments, a template polynucleotide for inserting an HLA-E fusion protein into an endogenous B2M locus comprises the sequence set forth in SEQ ID NO: 137.

A polynucleotide can be introduced into a cell as part of a vector molecule having additional sequences such as, for example, replication origins, promoters and genes encoding antibiotic resistance. Moreover, template polynucleotides can be introduced as naked nucleic acid, as nucleic acid complexed with materials such as a liposome, nanoparticle or poloxamer, or can be delivered by viruses (e.g., adenovirus, AAV, herpesvirus, retrovirus, lentivirus and integrase defective lentivirus (IDLV)).

In other aspects, the template polynucleotide is delivered by viral and/or non-viral gene transfer methods. In some embodiments, the template polynucleotide is delivered to the cell via an adeno associated virus (AAV), such as any described herein.

In some embodiments, the template polynucleotide is comprised in a viral vector, and is at least at or about 2500, 2750, 3000, 3250, 3500, 3750, 4000, 4250, 4500, 4760, 5000, 5250, 5500, 5750, 6000, 7000, 7500, 8000, 9000 or 10000 nucleotides in length, or any value between any of the foregoing. In some embodiments, the polynucleotide is comprised in a viral vector, and is between at or about 2500 and at or about 5000 nucleotides, at or about 3500 and at or about 4500 nucleotides, or at or about 3750 nucleotides and at or about 4250 nucleotides in length. In some embodiments, the polynucleotide is comprised in a viral vector, and is at or about 2500, 2750, 3000, 3250, 3500, 3750, 4000, 4250, 4500, 4760, 5000, 5250, 5500, 5750, 6000, 7000, 7500, 8000, 9000 or 10000 nucleotides in length.

In some embodiments, the template polynucleotide is an adenovirus vector, e.g., an AAV vector, e.g., a ssDNA molecule of a length and sequence that allows it to be packaged in an AAV capsid. The vector may be, e.g., less than 5 kb and may contain an ITR sequence that promotes packaging into the capsid. The vector may be integration-deficient. In some embodiments, the template polynucleotide comprises about 150 to 1000 nucleotides of homology on either side of the transgene and/or the target site. In some embodiments, the template polynucleotide comprises about 100, 150, 200, 300, 400, 500, 600, 700, 800, 900, 1000, 1500, or 2000 nucleotides 5′ of the target site or transgene, 3′ of the target site or transgene, or both 5′ and 3′ of the target site or transgene. In some embodiments, the template polynucleotide comprises at least 100, 150, 200, 300, 400, 500, 600, 700, 800, 900, 1000, 1500, or 2000 nucleotides 5′ of the target site or transgene, 3′ of the target site or transgene, or both 5′ and 3′ of the target site or transgene. In some embodiments, the template polynucleotide comprises at most 100, 150, 200, 300, 400, 500, 600, 700, 800, 900, 1000, 1500, or 2000 nucleotides 5′ of the target site or transgene, 3′ of the target site or transgene, or both 5′ and 3′ of the target site or transgene.

In some embodiments, the template polynucleotide is a lentiviral vector, e.g., an IDLV (integration deficiency lentivirus).

The double-stranded template polynucleotides described herein may include one or more non-natural bases and/or backbones. In particular, insertion of a template polynucleotide with methylated cytosines may be carried out using the methods described herein to achieve a state of transcriptional quiescence in a region of interest.

In some embodiments, the transgene further encodes one or more marker(s). In some embodiments, the one or more marker(s) is a transduction marker, surrogate marker and/or a selection marker.

In some embodiments, the marker is a transduction marker or a surrogate marker. A transduction marker or a surrogate marker can be used to detect cells that have been introduced with the polynucleotide, e.g., a polynucleotide encoding a recombinant HLA-E fusion protein. In some embodiments, the transduction marker can indicate or confirm modification of a cell. In some embodiments, the surrogate marker is a protein that is made to be co-expressed on the cell surface with the recombinant HLA-E fusion protein. In particular embodiments, such a surrogate marker is a surface protein that has been modified to have little or no activity.

In some embodiments, the polynucleotide contains the structure: [5′ homology arm]-[transgene sequence]-[3′ homology arm]. In some embodiments, the polynucleotide contains the structure: [5′ homology arm]-[multicistronic element]-[transgene sequence]-[3′ homology arm]. In some embodiments, the polynucleotide contains the structure: [5′ homology arm]-[promoter]-[transgene sequence]-[3′ homology arm].

Construction of such expression cassettes, following the teachings of the present specification, utilizes methodologies well known in molecular biology (see, for example, Ausubel or Maniatis). Before use of the expression cassette to generate a transgenic animal, the responsiveness of the expression cassette to the stress-inducer associated with selected control elements can be tested by introducing the expression cassette into a suitable cell line (e.g., primary cells, transformed cells, or immortalized cell lines).

### D. Delivery of Agents for Genetic Disruption and Template Polynucleotides

In some embodiments, the genetic disruption, such as a genetic disruption at an endogenous TRAC and/or B2M locus is carried out by delivering or introducing one or more agent(s) capable of inducing a genetic disruption, e.g., Cas9, Cas12a, and/or gRNA components, to a cell, using any of a number of known delivery method or vehicle for introduction or transfer to cells, for example, using viral delivery vectors, or any of the known methods or vehicles for delivering Cas molecules and gRNAs. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505. In some embodiments, nucleic acid sequences encoding one or more components of one or more agent(s) capable of inducing a genetic disruption is introduced into the cells, e.g., by any methods for introducing nucleic acids into a cell described herein or known. In some embodiments, a vector encoding components of one or more agent(s) capable of inducing a genetic disruption such as a CRISPR guide RNA and/or a Cas enzyme can be delivered into the cell.

In some embodiments, the one or more agent(s) capable of inducing a genetic disruption, e.g., one or more agent(s) that is a Cas9/gRNA and/or Cas12a/gRNA, is introduced into the cell as a ribonucleoprotein (RNP) complex. RNP complexes include a sequence of ribonucleotides, such as an RNA or a gRNA molecule, and a protein, such as a Cas9 protein, a Cas12a protein, or variant thereof. For example, the Cas protein is delivered as RNP complex that comprises a Cas protein and a gRNA molecule targeting the target sequence, e.g., using electroporation or other physical delivery method. In some embodiments, the RNP is delivered into the cell via electroporation or other physical means, e.g., particle gun, Calcium Phosphate transfection, cell compression or squeezing. In some embodiments, the RNP can cross the plasma membrane of a cell without the need for additional delivery agents (e.g., small molecule agents, lipids, etc.). In some embodiments, delivery of the one or more agent(s) capable of inducing genetic disruption, e.g., CRISPR/Cas, as an RNP offers an advantage that the targeted disruption occurs transiently, e.g., in cells to which the RNP is introduced, without propagation of the agent to cell progenies. For example, delivery by RNP minimizes the agent from being inherited to its progenies, thereby reducing the chance of off-target genetic disruption in the progenies. In such cases, the genetic disruption and the integration of transgene can be inherited by the progeny cells, but without the agent itself, which may further introduce off-target genetic disruptions, being passed on to the progeny cells.

Agent(s) and components capable of inducing a genetic disruption, e.g., a Cas9 molecule and/or a Cas12a molecule and gRNA molecule, can be introduced into target cells in a variety of forms using a variety of delivery methods and formulations, as set forth in Tables 2 and 3, or methods described in, e.g., WO 2015/161276; US 2015/0056705, US 2016/0272999, US 2017/0211075; or US 2017/0016027. As described further herein, the delivery methods and formulations can be used to deliver template polynucleotides and/or other agents to the cell (such as those required for engineering the cells) in prior or subsequent steps of the methods described herein. When a Cas protein or gRNA component is encoded as DNA for delivery, the DNA may typically but not necessarily include a control region, e.g., comprising a promoter, to effect expression. Exemplary promoters for Cas9 molecule sequences include, e.g., CMV, EF1α, EFS, MSCV, PGK, or CAG promoters. Useful promoters for gRNAs include, e.g., H1, EF-1α, tRNA or U6 promoters. Promoters with similar or dissimilar strengths can be selected to tune the expression of components. Sequences encoding a Cas molecule may comprise a nuclear localization signal (NLS), e.g., an SV40 NLS. In some embodiments a promoter for a Cas molecule or a gRNA molecule may be, independently, inducible, tissue specific, or cell specific. In some embodiments, an agent capable of inducing a genetic disruption is introduced RNP complexes.

TABLE 2
Exemplary Delivery Methods

| Elements | | |
|---|---|---|
| Cas | gRNA | |
| Molecule(s) | molecule(s) | Comments |
| DNA | DNA | In this embodiment, a Cas molecule and a gRNA are transcribed |
| | | from DNA. In this embodiment, they are encoded on separate |
| | | molecules. |
| DNA | | In this embodiment, a Cas molecule and a gRNA are transcribed |
| | | from DNA, here from a single molecule. |
| DNA | RNA | In this embodiment, a Cas molecule is transcribed from DNA, and |
| | | a gRNA is provided as in vitro transcribed or synthesized RNA |
| mRNA | RNA | In this embodiment, a Cas molecule is translated from in vitro |
| | | transcribed mRNA, and a gRNA is provided as in vitro |
| | | transcribed or synthesized RNA. |
| mRNA | DNA | In this embodiment, a Cas molecule is translated from in vitro |
| | | transcribed mRNA, and a gRNA is transcribed from DNA. |
| Protein | DNA | In this embodiment, a Cas molecule is provided as a protein, and a |
| | | gRNA is transcribed from DNA. |
| Protein | RNA | In this embodiment, a Cas molecule is provided as a protein, and a |
| | | gRNA is provided as transcribed or synthesized RNA. |

TABLE 3
Comparison of Exemplary Delivery Methods

| | | Delivery | | | |
|---|---|---|---|---|---|
| | | into Non- | | | Type of |
| | | Dividing | Duration of | Genome | Molecule |
| Delivery Vector/Mode | | Cells | Expression | Integration | Delivered |
| Physical (e.g., electroporation, | | YES | Transient | NO | Nucleic |
| particle gun, Calcium Phosphate | | | | | Acids and |
| transfection, cell compression or | | | | | Proteins |
| squeezing) | | | | | |
| Viral | Retrovirus | NO | Stable | YES | RNA |
| | Lentivirus | YES | Stable | YES/NO | RNA |
| | | | | with | |
| | | | | modifications | |
| | Adenovirus | YES | Transient | NO | DNA |
| | Adeno-Associated | YES | Stable | NO | DNA |
| | Virus (AAV) | | | | |
| | Vaccinia Virus | YES | Very | NO | DNA |
| | | | Transient | | |
| | Herpes Simplex Virus | YES | Stable | NO | DNA |
| Non-Viral | Cationic Liposomes | YES | Transient | Depends on | Nucleic |
| | | | | what is | Acids and |
| | | | | delivered | Proteins |
| | Polymeric | YES | Transient | Depends on | Nucleic |
| | Nanoparticles | | | what is | Acids and |
| | | | | delivered | Proteins |
| Biological | Attenuated Bacteria | YES | Transient | NO | Nucleic |
| Non-Viral | | | | | Acids |
| Delivery | Engineered | YES | Transient | NO | Nucleic |
| Vehicles | Bacteriophages | | | | Acids |
| | Mammalian Virus- | YES | Transient | NO | Nucleic |
| | like Particles | | | | Acids |
| | Biological liposomes: | YES | Transient | NO | Nucleic |
| | Erythrocyte Ghosts | | | | Acids |
| | and Exosomes | | | | |

In some embodiments, DNA encoding Cas molecules and/or gRNA molecules, or RNP complexes comprising a Cas molecule and/or gRNA molecules, can be delivered into cells by known methods or as described herein. For example, Cas9-encoding and/or gRNA-encoding DNA can be delivered, e.g., by vectors (e.g., viral or non-viral vectors), non-vector based methods (e.g., using naked DNA or DNA complexes), or a combination thereof. Similarly, Cas12a-encoding and/or gRNA-encoding DNA can be delivered, e.g., by vectors (e.g., viral or non-viral vectors), non-vector based methods (e.g., using naked DNA or DNA complexes), or a combination thereof. In some embodiments, the polynucleotide containing the agent(s) and/or components thereof is delivered by a vector (e.g., viral vector/virus or plasmid). The vector may be any described herein.

In some aspects, a CRISPR enzyme (e.g. Cas nuclease) in combination with (and optionally complexed with) a guide sequence is delivered to the cell. For example, one or more elements of a CRISPR system is derived from a type I, type II, or type III CRISPR system. For example, one or more elements of a CRISPR system are derived from a particular organism comprising an endogenous CRISPR system, such as Streptococcus pyogenes, Staphylococcus aureus or Neisseria meningitides.

In some embodiments, the polynucleotide containing the agent(s) and/or components thereof or RNP complex is delivered by a non-vector based method (e.g., using naked DNA or DNA complexes). For example, the DNA or RNA or proteins or combination thereof, e.g., ribonucleoprotein (RNP) complexes, can be delivered, e.g., by organically modified silica or silicate (Ormosil), electroporation, transient cell compression or squeezing (such as described in Lee, et al. (2012) Nano Lett 12: 6322-27, Kollmannsperger et al (2016) Nat Comm 7, 10372), gene gun, sonoporation, magnetofection, lipid-mediated transfection, dendrimers, inorganic nanoparticles, calcium phosphates, or a combination thereof.

In some embodiments, delivery via electroporation comprises mixing the cells with the Cas- and/or gRNA-encoding DNA or RNP complex in a cartridge, chamber or cuvette and applying one or more electrical impulses of defined duration and amplitude. In some embodiments, delivery via electroporation is performed using a system in which cells are mixed with the Cas- and/or gRNA-encoding DNA in a vessel connected to a device (e.g., a pump) which feeds the mixture into a cartridge, chamber or cuvette wherein one or more electrical impulses of defined duration and amplitude are applied, after which the cells are delivered to a second vessel.

In some embodiments, the delivery vehicle is a non-viral vector. In some embodiments, the non-viral vector is an inorganic nanoparticle. Exemplary inorganic nanoparticles include, e.g., magnetic nanoparticles (e.g., Fe₃MnO₂) and silica. The outer surface of the nanoparticle can be conjugated with a positively charged polymer (e.g., polyethylenimine, polylysine, polyserine) which allows for attachment (e.g., conjugation or entrapment) of payload. In some embodiments, the non-viral vector is an organic nanoparticle. Exemplary organic nanoparticles include, e.g., SNALP liposomes that contain cationic lipids together with neutral helper lipids which are coated with polyethylene glycol (PEG), and protamine-nucleic acid complexes coated with lipid. Exemplary lipids and polymers for gene transfer include those described in, for example, WO 2019/195492 and WO 2020/223535.

In some embodiments, the vehicle has targeting modifications to increase target cell update of nanoparticles and liposomes, e.g., cell specific antigens, monoclonal antibodies, single chain antibodies, aptamers, polymers, sugars, and cell penetrating peptides. In some embodiments, the vehicle uses fusogenic and endosome-destabilizing peptides/polymers. In some embodiments, the vehicle undergoes acid-triggered conformational changes (e.g., to accelerate endosomal escape of the cargo). In some embodiments, a stimulus-cleavable polymer is used, e.g., for release in a cellular compartment. For example, disulfide-based cationic polymers that are cleaved in the reducing cellular environment can be used.

In some embodiments, the delivery vehicle is a biological non-viral delivery vehicle. In some embodiments, the vehicle is an attenuated bacterium (e.g., naturally or artificially engineered to be invasive but attenuated to prevent pathogenesis and expressing the transgene (e.g., Listeria monocytogenes, certain Salmonella strains, Bifidobacterium longum, and modified Escherichia coli), bacteria having nutritional and tissue-specific tropism to target specific cells, bacteria having modified surface proteins to alter target cell specificity). In some embodiments, the vehicle is a genetically modified bacteriophage (e.g., engineered phages having large packaging capacity, less immunogenicity, containing mammalian plasmid maintenance sequences and having incorporated targeting ligands). In some embodiments, the vehicle is a mammalian virus-like particle. For example, modified viral particles can be generated (e.g., by purification of the “empty” particles followed by ex vivo assembly of the virus with the desired cargo). The vehicle can also be engineered to incorporate targeting ligands to alter target tissue-specificity. In some embodiments, the vehicle is a biological liposome. For example, the biological liposome is a phospholipid-based particle derived from human cells (e.g., erythrocyte ghosts, which are red blood cells broken down into spherical structures derived from the subject (e.g., tissue targeting can be achieved by attachment of various tissue or cell-specific ligands), or secretory exosomes—subject-derived membrane-bound nanovescicles (30-100 nm) of endocytic origin (e.g., can be produced from various cell types and can therefore be taken up by cells without the need for targeting ligands).

In some embodiments, RNA encoding Cas molecules and/or gRNA molecules, can be delivered into cells, e.g., target cells described herein, by known methods or as described herein. For example, Cas-encoding and/or gRNA-encoding RNA can be delivered, e.g., by microinjection, electroporation, transient cell compression or squeezing (such as described in Lee, et al. (2012) Nano Lett 12: 6322-27), lipid-mediated transfection, peptide-mediated delivery, e.g., cell-penetrating peptides, or a combination thereof.

In some embodiments, delivery via electroporation comprises mixing the cells with the RNA encoding Cas molecules and/or gRNA molecules in a cartridge, chamber or cuvette and applying one or more electrical impulses of defined duration and amplitude. In some embodiments, delivery via electroporation is performed using a system in which cells are mixed with the RNA encoding Cas molecules and/or gRNA molecules in a vessel connected to a device (e.g., a pump) which feeds the mixture into a cartridge, chamber or cuvette wherein one or more electrical impulses of defined duration and amplitude are applied, after which the cells are delivered to a second vessel.

In some embodiments, Cas molecules can be delivered into cells by known methods or as described herein. For example, Cas protein molecules can be delivered, e.g., by microinjection, electroporation, transient cell compression or squeezing (such as described in Lee, et al. (2012) Nano Lett 12: 6322-27), lipid-mediated transfection, peptide-mediated delivery, or a combination thereof. Delivery can be accompanied by DNA encoding a gRNA or by a gRNA.

In some embodiments, the one or more agent(s) capable of introducing a cleavage, e.g., a Cas/gRNA system, is introduced into the cell as a ribonucleoprotein (RNP) complex. RNP complexes include a sequence of ribonucleotides, such as an RNA or a gRNA molecule, and a protein, such as a Cas protein or variant thereof. For example, the Cas protein is delivered as RNP complex that comprises a Cas protein and a gRNA molecule targeting the target sequence, e.g., using electroporation or other physical delivery method. In some embodiments, the RNP is delivered into the cell via electroporation or other physical means, e.g., particle gun, calcium phosphate transfection, cell compression or squeezing.

In some embodiments, delivery via electroporation comprises mixing the cells with the Cas molecules with or without gRNA molecules in a cartridge, chamber or cuvette and applying one or more electrical impulses of defined duration and amplitude. In some embodiments, delivery via electroporation is performed using a system in which cells are mixed with the Cas molecules with or without gRNA molecules in a vessel connected to a device (e.g., a pump) which feeds the mixture into a cartridge, chamber or cuvette wherein one or more electrical impulses of defined duration and amplitude are applied, after which the cells are delivered to a second vessel.

In some embodiments, delivery via electroporation comprises mixing the cells with the Cas molecules with or without gRNA molecules in a cartridge, chamber or cuvette and applying one or more electrical impulses of defined duration and amplitude. In some embodiments, delivery via electroporation is performed using a system in which cells are mixed with the Cas molecules.

In some embodiments, the polynucleotide containing the agent(s) and/or components thereof is delivered by a combination of a vector and a non-vector based method. For example, a virosome comprises a liposome combined with an inactivated virus (e.g., HIV or influenza virus), which can result in more efficient gene transfer than either a viral or a liposomal method alone.

In some embodiments, more than one agent(s) or components thereof are delivered to the cell. For example, in some embodiments, agent(s) capable of inducing a genetic disruption of three or more locations in the genome, e.g., a target site at a TRAC locus and a target site at a B2M locus are delivered to the cell. In some embodiments, agent(s) and components thereof are delivered using one method. For example, in some embodiments, one or more agents, for example, for inducing a genetic disruption at a target site at a TRAC locus and a further genetic disruption at a target site at a B2M locus are delivered as a first agent, e.g., a first RNP, and a second agent, e.g., a second RNP, respectively. In some aspects, the two or more different RNP complexes, such as an RNP targeting a target site at a TRAC locus and a further RNP targeting a target site at a B2M locus are delivered together, such as electroporated together, for example, in one electroporation reaction.

In some embodiments, one or more polynucleotides other than the one or more agent(s) capable of inducing a genetic disruption and/or component thereof, e.g., one or more CRISPR-Cas combinations, such as a template polynucleotide for HDR-directed integration (such as any template polynucleotide described herein, e.g., in Section I.C), are delivered. In some embodiments, the polynucleotide, e.g., template polynucleotide, is delivered at the same time as one or more of the components of the Cas system. In some embodiments, the polynucleotide is delivered before or after (e.g., less than about 1 minute, 5 minutes, 10 minutes, 15 minutes, 30 minutes, 1 hour, 2 hours, 3 hours, 6 hours, 9 hours, 12 hours, 1 day, 2 days, 3 days, 1 week, 2 weeks, or 4 weeks) one or more of the components of the Cas system are delivered. In some embodiments, the polynucleotide, e.g., template polynucleotide, is delivered by a different means from one or more of the components of the Cas system, e.g., the Cas9 molecule or the Cas12a molecule component and/or the gRNA molecule component. The polynucleotide, e.g., template polynucleotide, can be delivered by any of the delivery methods described herein. For example, the polynucleotide, e.g., template polynucleotide, can be delivered by a viral vector, e.g., any described herein such as an AAV vector, and the Cas component and/or the gRNA molecule component can be delivered by electroporation. In some embodiments, the polynucleotide, e.g., template polynucleotide, includes one or more exogenous sequences, e.g., transgene sequences that encode a recombinant CAR, a recombinant HLA-E fusion protein and/or a portion thereof and/or other exogenous gene nucleic acid sequences.

In some embodiments, the polynucleotide, e.g., a polynucleotide such as a template polynucleotide encoding the recombinant CAR, are introduced into the cells in nucleotide form, e.g., as a polynucleotide or a vector. In particular embodiments, the polynucleotide contains a transgene that encodes the recombinant CAR or a portion thereof.

In some embodiments, the polynucleotide, e.g., a polynucleotide such as a template polynucleotide encoding the recombinant HLA-E fusion protein, are introduced into the cells in nucleotide form, e.g., as a polynucleotide or a vector. In particular embodiments, the polynucleotide contains a transgene that encodes the recombinant HLA-E fusion protein or a portion thereof.

In some embodiments, the polynucleotide, e.g., template polynucleotide, is introduced into the cell for engineering, in addition to the agent(s) capable of inducing a targeted genetic disruption, e.g., nuclease and/or gRNAs. In some embodiments, the polynucleotide(s) may be delivered prior to, simultaneously or after the agent(s) capable of inducing a targeted genetic disruption is introduced into a cell. In some embodiments, the polynucleotide(s) are delivered simultaneously with the agents. In some embodiments, the polynucleotides are delivered prior to the agents, for example, seconds to hours to days before the agents, including, but not limited to, 1 to 60 minutes (or any time therebetween) before the agents, 1 to 24 hours (or any time therebetween) before the agents or more than 24 hours before the agents. In some embodiments, the polynucleotides are delivered after the agents, seconds to hours to days after the agents, including immediately after delivery of the agent, e.g., between or between about between 30 seconds to 4 hours, such as about 30 seconds, 1 minute, 2 minutes, 3 minutes, 4 minutes, 5 minutes, 6 minutes, 6 minutes, 8 minutes, 9 minutes, 10 minutes, 15 minutes, 20 minutes, 30 minutes, 40 minutes, 50 minutes, 60 minutes, 90 minutes, 2 hours, 3 hours or 4 hours after delivery of the agents and/or preferably within 4 hours of delivery of the agents. In some embodiments, the polynucleotide is delivered more than 4 hours after delivery of the agents. In some embodiments, the polynucleotides are delivered after the agents, for example, including, but not limited to, within 1 second to 60 minutes (or any time therebetween) after the agents, 1 to 4 hours (or any time therebetween) after the agents or more than 4 hours after the agents.

In some embodiments, the polynucleotides, e.g., template polynucleotides, may be delivered using the same delivery systems as the agent(s) capable of inducing a targeted genetic disruption, e.g., nuclease and/or gRNAs. In some embodiments, the polynucleotides may be delivered using different same delivery systems as the agent(s) capable of inducing a targeted genetic disruption, e.g., nuclease and/or gRNAs. In some embodiments, the polynucleotide is delivered simultaneously with the agent(s). In other embodiments, the polynucleotide is delivered at a different time, before or after delivery of the agent(s). Any of the delivery method described herein in Section I.C (e.g., in Tables 2 and 3) for delivery of nucleic acids in the agent(s) capable of inducing a targeted genetic disruption, e.g., nuclease and/or gRNAs, can be used to deliver the polynucleotide.

In some embodiments, the one or more agent(s) and the polynucleotide are delivered in the same format or method. For example, in some embodiments, the one or more agent(s) and the polynucleotide are both comprised in a vector, e.g., viral vector. In some embodiments, the polynucleotide is encoded on the same vector backbone, e.g. AAV genome, plasmid DNA, as the Cas and gRNA. In some aspects, the one or more agent(s) and the polynucleotide are in different formats, e.g., ribonucleic acid-protein complex (RNP) for the Cas-gRNA agent and a linear DNA for the polynucleotide, but they are delivered using the same method. In some aspects, the one or more agent(s) and the polynucleotide are in different formats, e.g., ribonucleic acid-protein complex (RNP) for the Cas-gRNA agent and the polynucleotide is in contained in an AAV vector, and the RNP is delivered using a physical delivery method (e.g., electroporation) and the polynucleotide is delivered via transduction of AAV viral preparations. In some aspects, the polynucleotide is delivered immediately after, e.g., within about 1, 2, 3, 4, 5, 10, 20, 30, 40, 50 or 60 minutes after, the delivery of the one or more agent(s).

In some embodiments, the one or more agent(s) is or comprises a ribonucleoprotein (RNP) complex. In some embodiments, the concentration of each RNP incubated with, added to or contacted with the cells for engineering is independently at a concentration of at or about 0.01, 0.025, 0.05, 0.1, 0.25, 0.5, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 4, 5, 6, 7, 7.5, 8, 9, 10 μM, or a range defined by any two of the foregoing values. In some aspects, the concentration of each RNP is independently between at or about 0.025 μM and at or about 5 μM, between at or about 0.025 μM and at or about 2.5 μM, between at or about 0.025 μM and at or about 1 μM, 0.025 μM and at or about 0.5 μM, between at or about 0.025 μM and at or about 0.1 μM, or between at or about 0.025 μM and at or about 0.25 μM. In some aspects, the concentration of each RNP is independently between at or about 1 μM and at or about 5 μM. In some aspects, the concentration of each RNP is independently between at or about 1.5 μM and at or about 2.5 μM. In some aspects, the concentration of each RNP is independently between at or about 0.05 μM and at or about 1 μM. In some aspects, the concentration of each RNP is independently between at or about 0.025 μM and at or about 0.25 μM.

In some embodiments, the one or more agent(s) is or comprises a ribonucleoprotein (RNP) complex. In some embodiments, the total concentration of RNPs incubated with, added to or contacted with the cells for engineering is at a concentration of at or about 0.01, 0.025, 0.05, 0.1, 0.25, 0.5, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 4, 5, 6, 7, 7.5, 8, 9, 10, 20, 30, 40, or 50 μM, or a range defined by any two of the foregoing values. In some aspects, the total concentration of RNPs is between at or about 0.025 μM and at or about 5 μM, between at or about 0.025 μM and at or about 2.5 μM, between at or about 0.025 μM and at or about 1 μM, 0.025 μM and at or about 0.5 μM, between at or about 0.025 μM and at or about 0.1 μM, or between at or about 0.025 μM and at or about 0.25 μM. In some aspects, the total concentration of RNP is between at or about 1 μM and at or about 5 μM.

In some embodiments, in the RNP complex, the ratio, e.g. the molar ratio, of the gRNA and the Cas molecule or other nucleases is at or about 5:1, 4:1, 3:1, 2:1, 1:1, 1:2, 1:3, 1:4 or 1:5, or a range defined by any two of the foregoing values. In some embodiments, in the RNP complex, the ratio, e.g., molar ratio, of the gRNA and the Cas molecule or other nucleases is at or about 3:1, 2.9:1, 2.8:1, 2.7:1, 2.6:1, 2.5:1, 2.4:1, 2.3:1, 2.2:1, 2.1:1, 2:1 or 1:1, or a range defined by any two of the foregoing values.

In some embodiments, the polynucleotide is a linear or circular polynucleotide, such as a linear or circular DNA or linear RNA, and can be delivered using any of the methods described in Section I.D herein (e.g., Tables 2 and 3) for delivering polynucleotides into the cell.

In particular embodiments, the polynucleotide, e.g., the template polynucleotide, are introduced into the cells in nucleotide form, e.g., as or within a non-viral vector. In some embodiments, the non-viral vector is or includes a polynucleotide, e.g., a DNA or RNA polynucleotide, that is suitable for transduction and/or transfection by any suitable and/or known non-viral method for gene delivery, such as but not limited to microinjection, electroporation, transient cell compression or squeezing (e.g., as described in Lee, et al. (2012) Nano Lett 12: 6322-27), lipid-mediated transfection, peptide-mediated delivery, e.g., cell-penetrating peptides, or a combination thereof. In some embodiments, the non-viral polynucleotide is delivered into the cell by a non-viral method described herein, such as a non-viral method listed in Table 3 herein.

In some embodiments, the polynucleotide sequence can be comprised in a vector molecule containing sequences that are not homologous to the region of interest in the genomic DNA.

In some embodiments, the polynucleotides and sequences encoding the one or more agents may be on the same vector, for example an AAV vector. In some embodiments, the polynucleotides are delivered using an AAV vector and the one or more agents for inducing a genetic disruption, e.g., one or more CRISPR-Cas combination, are delivered as a different form, e.g., as mRNAs encoding the nucleases and/or gRNAs. In some embodiments, the polynucleotides and nucleases are delivered using the same type of method, e.g., a viral vector, but on separate vectors. In some embodiments, the polynucleotides are delivered in a different delivery system as the agents capable of inducing a genetic disruption, e.g., nucleases and/or gRNAs. In some embodiments, the polynucleotide is excised from a vector backbone in vivo, e.g., it is flanked by gRNA recognition sequences. In some embodiments, the polynucleotide is on a separate polynucleotide molecule as the Cas and gRNA. In some embodiments, the Cas and the gRNA are introduced in the form of a ribonucleoprotein (RNP) complex, and the polynucleotide is introduced as a polynucleotide molecule, e.g., in a vector or a linear polynucleotide, e.g., linear DNA.

In some embodiments the vector or construct can contain a promoter and/or enhancer or regulatory elements to regulate expression of the encoded recombinant receptor. In some examples the promoter and/or enhancer or regulatory elements can be condition-dependent promoters, enhancers, and/or regulatory elements. In some examples these elements drive expression of the transgene. In some examples, the CAR transgene can be operatively linked to a promoter, such as an EF1alpha promoter with an HTLV1 enhancer (SEQ ID NO: 56). In some examples, the CAR transgene is operatively linked to a Woodchuck Hepatitis Virus (WHP) Posttranscriptional Regulatory Element (WPRE; SEQ ID NO: 57), located downstream of the transgene.

In some embodiments, the vector or construct can contain a single promoter that drives the expression of one or more nucleic acid molecules. In some embodiments, such nucleic acid molecules, e.g., transcripts, can be multicistronic (bicistronic or tricistronic, see e.g., U.S. Pat. No. 6,060,273). For example, in some embodiments, transcription units can be engineered as a bicistronic unit containing an IRES (internal ribosome entry site), which allows coexpression of gene products (e.g., encoding a first and second chimeric receptor) by a message from a single promoter.

Alternatively, in some cases, a single promoter may direct expression of an RNA that contains, in a single open reading frame (ORF), two or three genes (e.g., encoding a first and second binding molecules, e.g., antibody recombinant receptor) separated from one another by sequences encoding a self-cleavage peptide (e.g., 2A cleavage sequences) or a protease recognition site (e.g., furin). The ORF thus encodes a single polypeptide, which, either during (in the case of T2A) or after translation, is cleaved into the individual proteins. In some cases, the peptide, such as T2A, can cause the ribosome to skip (ribosome skipping) synthesis of a peptide bond at the C-terminus of a 2A element, leading to separation between the end of the 2A sequence and the next peptide downstream (see, for example, de Felipe. Genetic Vaccines and Ther. 2:13 (2004) and deFelipe et al. Traffic 5:616-626 (2004)). Many 2A elements are known. Examples of 2A sequences that can be used in the methods and polynucleotides disclosed herein, without limitation, 2A sequences from the foot-and-mouth disease virus (F2A, e.g., SEQ ID NO: 58 or SEQ ID NO: 59), equine rhinitis A virus (E2A, e.g., SEQ ID NO: 60 or SEQ ID NO: 61), Thosea asigna virus (T2A, e.g., SEQ ID NO: 62, SEQ ID NO: 63, or SEQ ID NO: 64), and porcine teschovirus-1 (P2A, e.g., SEQ ID NO: 65 or SEQ ID NO: 66) as described in U.S. Patent Publication No. 20070116690. In some embodiments, the one or more different or separate promoters drive the expression of one or more nucleic acid molecules encoding the one or more binding molecules, e.g., recombinant receptors.

In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

### E. Incubation, Cultivation and Harvesting

In some embodiments, the provided methods involve incubating the immune cells, e.g., T cells. In some embodiments, the incubating is carried out after the introducing of the one or more gene-editing agents.

In some embodiments, the incubating can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to stimulate the immune cells. In some embodiments, the cultivating is under conditions to maintain a target amount of carbon dioxide in the cell culture. In some aspects, the amount of carbon dioxide (CO₂) is between 10% and 0% (v/v) of said gas, such as between 8% and 2% (v/v) of said gas, for example an amount of or about 5% (v/v) CO₂.

In some embodiments, the incubating is carried out in a cell medium. In some embodiments, the cell medium is a serum-free medium. In some embodiments, the cell medium can include one or more recombinant cytokines. In some embodiments, the cell medium is a basal medium that does not include any added recombinant cytokines.

In particular embodiments, the one or more cytokines are human recombinant cytokines. In certain embodiments, the one or more cytokines bind to receptors that are expressed by T cells. In particular embodiments, the one or more cytokines include a member of the 4-alpha-helix bundle family of cytokines. In some embodiments, members of the 4-alpha-helix bundle family of cytokines include interleukin-2 (IL-2), interleukin-4 (IL-4), interleukin-7 (IL-7), interleukin-9 (IL-9), interleukin 12 (IL-12), interleukin 15 (IL-15), granulocyte colony-stimulating factor (G-CSF), and granulocyte-macrophage colony-stimulating factor (GM-CSF). In some embodiments, the one or more cytokines include IL-15. In particular embodiments, the one or more cytokines include IL-7. In particular embodiments, the one or more cytokines include IL-2. In particular embodiments, the one or more cytokines are selected from IL-2, IL-15, and IL-7. In particular embodiments, the cell medium contains recombinant IL-2, IL-15, and IL-7.

In certain embodiments, the amount or concentration of the one or more cytokines are measured and/or quantified with International Units (IU). International units may be used to quantify vitamins, hormones, cytokines, vaccines, blood products, and similar biologically active substances. In some embodiments, IU are or include units of measure of the potency of biological preparations by comparison to an international reference standard of a specific weight and strength, e.g., WHO 1st International Standard for Human IL-2, 86/504. International Units are the only recognized and standardized method to report biological activity units that are published and are derived from an international collaborative research effort. In particular embodiments, the IU for population, sample, or source of a cytokine may be obtained through product comparison testing with an analogous WHO standard product. For example, in some embodiments, the IU/mg of a population, sample, or source of human recombinant IL-2, IL-7, or IL-15 is compared to the WHO standard IL-2 product (NIBSC code: 86/500), the WHO standard IL-17 product (NIBSC code: 90/530), and the WHO standard IL-15 product (NIBSC code: 95/554), respectively.

In particular embodiments, the ED50 of recombinant human IL-2 or IL-15 is equivalent to the concentration required for the half-maximal stimulation of cell proliferation (XTT cleavage) with CTLL-2 cells. In certain embodiments, the ED50 of recombinant human IL-7 is equivalent to the concentration required for the half-maximal stimulation for proliferation of PHA-activated human peripheral blood lymphocytes. Details relating to assays and calculations of IU for IL-2 are discussed in Wadhwa et al., Journal of Immunological Methods (2013), 379 (1-2): 1-7; and Gearing and Thorpe, Journal of Immunological Methods (1988), 114 (1-2): 3-9; details relating to assays and calculations of IU for IL-15 are discussed in Soman et al. Journal of Immunological Methods (2009) 348 (1-2): 83-94.

In some embodiments, the cell medium contains IL-2, e.g., human recombinant IL-2, at a concentration between 1 IU/mL and 500 IU/mL, between 10 IU/mL and 250 IU/mL, between 50 IU/mL and 200 IU/mL, between 50 IU/mL and 150 IU/mL, between 75 IU/mL and 125 IU/mL, between 100 IU/mL and 200 IU/mL, or between 10 IU/mL and 100 IU/mL. In particular embodiments, the cell medium contains recombinant IL-2 at a concentration at or at about 50 IU/mL, 60 IU/mL, 70 IU/mL, 80 IU/mL, 90 IU/mL, 100 IU/mL, 110 IU/mL, 120 IU/mL, 130 IU/mL, 140 IU/mL, 150 IU/mL, 160 IU/mL, 170 IU/mL, 180 IU/mL, 190 IU/mL, or 100 IU/mL. In some embodiments, the cell medium contains about 100 IU/mL of recombinant IL-2, e.g., human recombinant IL-2.

In some embodiments, the cell medium contains recombinant IL-7, e.g., human recombinant IL-7, at a concentration between 100 IU/mL and 2,000 IU/mL, between 500 IU/mL and 1,000 IU/mL, between 100 IU/mL and 500 IU/mL, between 500 IU/mL and 750 IU/mL, between 750 IU/mL and 1,000 IU/mL, or between 550 IU/mL and 650 IU/mL. In particular embodiments, the cell medium contains IL-7 at a concentration at or at about 50 IU/mL, 100 IU/mL, 150 IU/mL, 200 IU/mL, 250 IU/mL, 300 IU/mL, 350 IU/mL, 400 IU/mL, 450 IU/mL, 500 IU/mL, 550 IU/mL, 600 IU/mL, 650 IU/mL, 700 IU/mL, 750 IU/mL, 800 IU/mL, 750 IU/mL, 750 IU/mL, 750 IU/mL, or 1,000 IU/mL. In particular embodiments, the cell medium contains about 600 IU/mL of IL-7, e.g., human recombinant IL-7.

In some embodiments, the cell medium contains recombinant IL-15, e.g., human recombinant IL-15, at a concentration between 1 IU/mL and 500 IU/mL, between 10 IU/mL and 250 IU/mL, between 50 IU/mL and 200 IU/mL, between 50 IU/mL and 150 IU/mL, between 75 IU/mL and 125 IU/mL, between 100 IU/mL and 200 IU/mL, or between 10 IU/mL and 100 IU/mL. In particular embodiments, the cell medium contains recombinant IL-15 at a concentration at or at about 50 IU/mL, 60 IU/mL, 70 IU/mL, 80 IU/mL, 90 IU/mL, 100 IU/mL, 110 IU/mL, 120 IU/mL, 130 IU/mL, 140 IU/mL, 150 IU/mL, 160 IU/mL, 170 IU/mL, 180 IU/mL, 190 IU/mL, or 200 IU/mL. In some embodiments, the cell medium contains about 100 IU/mL of recombinant IL-15, e.g., human recombinant IL-15.

In some embodiments, at least a portion of the incubating is carried out under conditions for recovery of the T cells from the gene editing, such as following electroporation of the cells. In certain embodiments, at least a portion of the incubating is performed under static conditions, such as conditions that do not involve centrifugation, shaking, rotating, rocking, or perfusion of media. In some embodiments, the incubating is performed under gentle mixing conditions, e.g., involving rocking. In some embodiments, the at least a portion of the incubating under static conditions is carried out for between or between about 2 hours and 30 hours, 2 hours and 26 hours, 2 hours and 22 hours, 2 hours and 18 hours, 2 hours and 14 hours, 2 hours and 10 hours, 2 hours and 6 hours, 2 hours and 4 hours, 4 hours and 30 hours, 4 hours and 26 hours, 4 hours and 22 hours, 4 hours and 18 hours, 4 hours and 14 hours, 4 hours and 10 hours, 4 hours and 6 hours, 6 hours and 30 hours, 6 hours and 26 hours, 6 hours and 22 hours, 6 hours and 18 hours, 6 hours and 14 hours, 6 hours and 10 hours, 10 hours and 30 hours, 10 hours and 26 hours, 10 hours and 22 hours, 10 hours and 18 hours, 10 hours and 14 hours, 14 hours and 30 hours, 14 hours and 26 hours, 14 hours and 22 hours, 14 hours and 18 hours, 18 hours and 30 hours, 18 hours and 26 hours, 18 hours and 22 hours, 22 hours and 30 hours, 22 hours and 26 hours, or 26 hours and 30 hours, each inclusive.

In some embodiments, at least a portion of the incubating is under cultivating conditions for expansion of the cells. In some embodiments, the cultivating is carried out under conditions to induce expansion of the immune cells, e.g., T cells. In particular embodiments, the cultivating conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to promote growth, division, and/or expansion of the immune cells, e.g., T cells. In some embodiments, the cultivating is carried out for a time period until a desired or threshold density, concentration, or number of cells is achieved.

In some embodiments, the cultivating is carried out in a bioreactor. Examples of suitable bioreactors for the cultivating include GE Xuri W25, GE Xuri W5, Sartorius BioSTAT RM 20|50, Finesse SmartRocker Bioreactor Systems, and Pall XRS Bioreactor Systems. In some embodiments, the bioreactor is used to perfuse and/or mix the immune cells, e.g., T cells, during at least a portion of the cultivating.

In some embodiments, the cultivating occurs in an incubator. In some embodiments, the immune cells, e.g., T cells, are transferred into a container for the cultivating. In some embodiments, the container is a vial. In particular embodiments, the container is a bag. In some embodiments, the immune cells, e.g., T cells, are transferred into the container under closed or sterile conditions. In some embodiments, the container, e.g., the vial or bag, is then placed into an incubator for all or a portion of the cultivating. In particular embodiments, the incubator is set at, at about, or at least 16° C., 24° C., or 35° C. In some embodiments, the incubator is set at 37° C., at about at 37° C., or at 37° C.±2° C., ±1° C., ±0.5° C., or ±0.1° C.

In some embodiments, the incubation is carried out until a threshold number of population doublings has occurred. In some embodiments, the incubation under conditions for cultivating the cells is carried out until 2, 3, 4, 5, 6, 7, 8, 9, 10 or more population doublings has occurred. In some embodiments, the incubation under conditions for cultivating the cells is carried out until 2 to 8 population doublings has occurred. In some embodiments, the incubation under conditions for cultivating the cells is carried out until 4 to 8 population doublings has occurred. In some embodiments, the incubation under conditions for cultivating the cells is carried out until 6 or 7 population doublings has occurred.

In certain embodiments, the cultivating is for, for about, or for at least 18 hours, 24 hours, 30 hours, 36 hours, 40 hours, 48 hours, 54 hours, 60 hours, 72 hours, 84 hours, 96 hours, or more than 96 hours. In some embodiments, the cultivating is performed for an amount of time between 30 minutes and 2 hours, between 1 hour and 8 hours, between 6 hours and 12 hours, between 12 hours and 18 hours, between 16 hours and 24 hours, between 18 hours and 30 hours, between 24 hours and 48 hours, between 24 hours and 72 hours, between 42 hours and 54 hours, between 60 hours and 120 hours between 96 hours and 120 hours, between 90 hours and between 1 days and 7 days, between 3 days and 8 days, between 1 day and 3 days, between 4 days and 6 days, or between 4 days and 5 days. In some embodiments, the cultivating is carried out for no more than 14 days. In some embodiments, the cultivating is carried out for no more than 12 days. In some embodiments, the cultivating is carried out for no more than 10 days. In some embodiments, the cultivating is carried out for no more than 8 days. In some embodiments, the cultivating is carried out for no more than 6 days. In some embodiments, the cultivating is carried out for no more than 5 days. In some embodiments, the cultivating is carried out for between or between about 12 hours and 36 hours, inclusive. In some embodiments, the cultivating is carried out for between or between about 18 hours and 30 hours, inclusive. In some embodiments, the cultivating is carried out for between or between about 22 hours and 26 hours, inclusive. In particular embodiments, the cultivating is for or for about 24 hours.

In some embodiments, the provided methods involve harvesting the genetically engineered immune cells, e.g., T cells, expressing the recombinant protein. In some embodiments, the harvesting is carried out following the engineering. In some embodiments, the harvesting is performed carried out following the cultivating.

In some embodiments, one or more polishing step of the harvested cells can be carried out. In some embodiments, the polishing step is carried out to enrich for successfully engineered cells by removing or depleting cells that have not been engineered. In some embodiments, polishing of the engineered cells is carried out by depleting CD3+ T cells to remove T cells that have not been edited by disruption of the TRAC locus. In some embodiments, a selection step (e.g., polishing step) is useful for increasing product control and/or decreasing between engineering process variance. In some embodiments, the engineered T cells are depleted for CD3+ T cells, such as by immunoaffinity-based selection for CD3+ T cells. In some embodiments, the immunoaffinity-based selection involves contacting the cells with an antibody or antigen-binding fragment thereof directed against the target antigen (e.g., CD3). In certain embodiments, the immunoaffinity reagents are immobilized on the outside surface of a bead. In particular embodiments, the bead is a magnetic bead. In some embodiments, the depleting cells bound to the immunoaffinity reagent involves exposing the cells to a magnetic field, in which the cells not bound to the magnet are recovered as CD3+ depleted cells and harvested. In some embodiments, the affinity-based selection is via magnetic-activated cell sorting (MACS) (Miltenyi Biotech, Auburn, CA). Magnetic Activated Cell Sorting (MACS), e.g., CliniMACS systems are capable of high-purity selection of cells having magnetized particles attached thereto.

In some embodiments, the provided methods involve formulating the harvested genetically engineered immune cells, e.g., T cells. In some embodiments, the harvested genetically engineered immune cells, e.g., T cells, are formulated in a container, such as a bag or vial.

In some embodiments, the harvested genetically engineered immune cells, e.g., T cells, are formulated for administration to a subject. In some embodiments, the harvested genetically engineered immune cells, e.g., T cells, are formulated in a pharmaceutically acceptable buffer, which may, in some aspects, include a pharmaceutically acceptable carrier or excipient. In some embodiments, the harvested genetically engineered immune cells, e.g., T cells, are formulated in the presence of a pharmaceutically acceptable excipient.

In some embodiments, the harvested genetically engineered immune cells, e.g., T cells, are formulated for cryopreservation. In some embodiments, the harvested genetically engineered immune cells, e.g., T cells, are formulated in the presence of a cryoprotectant. In some embodiments, the harvested genetically engineered immune cells, e.g., T cells, are formulated with a cyropreservative solution that contains 1.0% to 30% DMSO solution, such as a 5% to 20% DMSO solution or a 5% to 10% DMSO solution. In some embodiments, the cryopreservation solution is or contains, for example, PBS containing 20% DMSO and 8% human serum albumin (HSA), or other suitable cell freezing media. In some embodiments, the cryopreservative solution is or contains, for example, at least or about 7.5% DMSO. In some embodiments, the harvested genetically engineered immune cells, e.g., T cells, are frozen, e.g., cryoprotected or cryopreserved, in media and/or solution with a final concentration of or of about 12.5%, 12.0%, 11.5%, 11.0%, 10.5%, 10.0%, 9.5%, 9.0%, 8.5%, 8.0%, 7.5%, 7.0%, 6.5%, 6.0%, 5.5%, or 5.0% DMSO, or between 1% and 15%, between 6% and 12%, between 5% and 10%, or between 6% and 8% DMSO. In particular embodiments, the harvested genetically engineered immune cells, e.g., T cells, are frozen, e.g., cryoprotected or cryopreserved, in media and/or solution with a final concentration of or of about 5.0%, 4.5%, 4.0%, 3.5%, 3.0%, 2.5%, 2.0%, 1.5%, 1.25%, 1.0%, 0.75%, 0.5%, or 0.25% HSA, or between 0.1% and −5%, between 0.25% and 4%, between 0.5% and 2%, or between 1% and 2% HSA.

### II. EXEMPLARY FEATURES OF GENETICALLY ENGINEERED CELLS

In some embodiments, cells and compositions according to the provided disclosure (e.g. cells comprising a modified TRAC locus comprising a transgene sequence encoding a recombinant CAR or a portion thereof, and/or a modified B2M locus comprising a transgene sequence encoding a recombinant HLA-E fusion protein or a portion thereof) exhibit an altered immunogenic profile compared to a corresponding or reference cell or composition.

Graft-versus-host disease (GVHD), also known as graft versus host disease, is a common complication following an allogeneic tissue or cell transplant. It is commonly associated with stem cell or bone marrow transplant but the term also applies to other forms of tissue or cell therapy graft. Immune cells (white blood cells) in the tissue (the graft) recognize the recipient (the host) as “foreign”. The transplanted immune cells then attack the host's body cells. GVHD can also occur after a blood transfusion if the blood products used have not been irradiated or treated with an approved pathogen reduction system.

In some aspects, the provided embodiments are based on observations that the efficacy of adoptive cell therapy may be limited by the development of an immune response in the subject to the cells and/or construct administered. In some embodiments, exogenous engineered cells when administered to a subject can be recognized by a host's immune response as foreign. In some embodiments, such recognition can occur when cells are allogenic to the host subject to which they are administered. In some embodiments, such recognition can occur even when the cells are autologous to the host subject to which they are administered, such as when the cells are engineered with a recombinant molecule or receptor (e.g. CAR) that is not native to or normally expressed by the cells. In some embodiments, development of a host immune response to administered cells, either allogenic or autologous cells, can result in host-versus-graft responses that can lead to rejection of adoptively transferred cells.

In some embodiments, the provided engineered cells, compositions and methods can be used regardless of the HLA type or subtype of the subject, which can, in some aspects, permit “off-the-shelf” delivery to a wider variety of recipients. In some embodiments, the provided compositions and methods can be used to provide adoptive cell therapy using allogeneic cells engineered to treat a disease or disorder. In some cases, using allogeneic cells can provide certain advantages. In some embodiments, cells with known safety and efficacy profiles can be prepared for a wider variety of patients. For example, cells can be derived from a healthy donor and delivered to a subject that may be too sick to provide cells suitable for genetic engineering. In some cases, a subject may have a defect or disease in the cells or cell type typically used for a particular adoptive cell therapy regimen, such that cells from a healthy donor can be used that replace or supplement the diseased cells. In some cases, the ability to engineer or administer allogeneic cells permits the preparation of cells in advance, which can reduce the time needed before being delivered to a patient. In some cases, the engineered allogenic cells may present lower risks of causing graft-versus-host disease or host-versus-graft disease.

In some embodiments, the provided cells and compositions are less immunogenic and/or result in a reduced degree of recognition of the engineered cell by the host immune system upon administration of the cells. In some embodiments, the provided cells and compositions result in a reduced risk of development of graft-vs-host and/or host-vs-graft disease. In some embodiments, the provided cells and compositions are more cytotoxic and/or result in increased killing of tumor cells upon administration of the cells. In some embodiments, the provided cells and compositions exhibit increased efficiency and/or result in longer utility in the killing of tumor cells upon administration of the cells. In some cases, reference to a “reference cell” or “reference composition” (and/or a “corresponding cell” or a “corresponding composition”), may refer to a cell or composition (such as a T cell or composition of T cells) that are obtained, isolated, generated, produced and/or incubated under the same or substantially the same conditions, except that the such cells do not have a reduction, deletion, elimination, knockout or disruption in expression of the TRAC and B2M gene and/or do not express a recombinant CAR and/or HLA-E fusion protein. In some aspects, except for not containing introduction of the CAR and/or the recombinant HLA-E fusion protein and/or genetic disruption of the TRAC and/or B2M genes, such cells or T cells are treated identically or substantially identically as T cells or cells that have been introduced with CAR and/or the recombinant HLA-E fusion protein and/or genetic disruption of the TRAC and/or B2M genes, such that any one or more conditions that can influence the activity or properties of the cell is not varied or not substantially varied between the cells other than the introduction of the agent.

In some embodiments, the reduction, disruption, deletion or elimination of expression of the TRAC and/or B2M genes in the provided engineered cells does not impair or alter the function or activity of the recombinant receptor compared to a reference cell or composition. In some embodiments, the recombinant receptor retains specific binding to the antigen. In some embodiments, the recombinant receptor retains activating or stimulating activity, upon antigen binding, to induce cytotoxicity, proliferation, survival or cytokine secretion in cells. In some embodiments, the engineered cells of the provided compositions retain a functional property or activity compared to a corresponding or reference composition when assessed under the same conditions. In some embodiments, the cells retain cytotoxicity, proliferation, survival or cytokine secretion compared to such a corresponding or reference composition.

### III. CHIMERIC ANTIGEN RECEPTORS

In some embodiments, the provided genetically engineered T cells contain or are engineered to contain a chimeric antigen receptor (CAR). The CAR generally includes an extracellular domain comprising an extracellular binding domain (also called “extracellular antigen binding domain”) directed against an antigen or antigens, in which the extracellular domain is linked to one or more intracellular signaling components, in some aspects via linkers and/or transmembrane domain(s). In some embodiments, the extracellular binding domain provides a means for binding an antigen or antigens. In some embodiments, the extracellular binding domain includes an antibody or antibody fragment that provides specificity for a desired antigen or antigens. In some aspects, the recombinant receptor, e.g., CAR, further includes a spacer and/or a transmembrane domain or portion. In some aspects, the spacer and/or transmembrane domain can link the extracellular portion containing the antigen binding domain and the intracellular signaling region(s) or domain(s). In some embodiments, the CAR includes in order from N- to C-terminus: the extracellular binding domain, a spacer, a transmembrane domain, and an intracellular signaling domain. In such embodiments, the spacer is interposed between the extracellular binding domain and the transmembrane domain.

In some embodiments, the intracellular signaling domain is a stimulating or an activating intracellular domain portion, such as a T cell stimulating or activating domain, providing a primary activation signal or primary signal. In some embodiments, the intracellular signaling domain comprises an intracellular signaling domain capable of inducing a primary activation signal in a T cell. In some embodiments, the intracellular signaling domain is a domain from a T cell receptor (TCR) component and/or comprises an immunoreceptor tyrosine-based activation motif (ITAM). In some embodiments, the intracellular signaling domain is a cytoplasmic signaling domain of a CD3-zeta (CD3ζ) chain, for instance a human CD3ζ chain. In some embodiments, the intracellular signaling domain contains or additionally contains a costimulatory signaling domain to facilitate effector functions. In some embodiments, the intracellular signaling region further comprises a costimulatory signaling region, such as an intracellular signaling domain of a T cell costimulatory molecule or a signaling portion thereof. In some embodiments, the costimulatory signaling region is between the transmembrane region and the intracellular signaling domain. Upon specific binding to the molecule, e.g., antigen, the receptor generally delivers an immunostimulatory signal, such as an ITAM-transduced signal, into the cell, thereby promoting an immune response targeted to the disease or condition. In some embodiments, chimeric receptors when genetically engineered into immune cells can modulate T cell activity, and, in some cases, can modulate T cell differentiation or homeostasis, thereby resulting in genetically engineered cells with improved longevity, survival and/or persistence in vivo, such as for use in adoptive cell therapy methods.

Exemplary antigen receptors, including CARs, and methods for engineering and introducing such receptors into cells, include those described, for example, in international patent application publication numbers WO200014257, WO2013126726, WO2012/129514, WO2014031687, WO2013/166321, WO2013/071154, WO2013/123061, U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4): 388-398; Davila et al. (2013) PloS ONE 8(4): e61338; Turtle et al., Curr. Opin. Immunol., 2012 October; 24(5): 633-39; Wu et al., Cancer, 2012 March 18(2): 160-75. In some aspects, the antigen receptors include a CAR as described in U.S. Pat. No. 7,446,190, and those described in International Patent Application Publication No.: WO/2014055668 Al. Examples of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, Kochenderfer et al., 2013, Nature Reviews Clinical Oncology, 10, 267-276 (2013); Wang et al. (2012) J. Immunother. 35(9): 689-701; and Brentjens et al., Sci Transl Med. 2013 5(177). See also WO2014031687, U.S. Pat. Nos. 8,339,645, 7,446,179, US 2013/0149337, U.S. Pat. Nos. 7,446,190, and 8,389,282.

The CAR generally includes an extracellular antigen binding domain, such as a portion of an antibody molecule, generally a variable heavy (VH) chain region and/or variable light (VL) chain region of an antibody. Among the provided receptors are CARs in which the extracellular binding domain is composed of variable region sequences (e.g., variable heavy chain and variable light chain sequences) of antibodies or antigen-binding fragments thereof. In some embodiments, the extracellular binding domain is an scFv antibody fragment. In some embodiments, the antibody or antigen-binding portion thereof is expressed on cells as part of a chimeric antigen receptor (CAR), that binds, such as specifically binds, to the antigen (e.g., CD19). In some embodiments, the CAR contains an extracellular domain, a transmembrane domain, and an intracellular signaling domain. In some embodiments, the CAR also includes a spacer domain (e.g. hinge domain) separating the extracellular binding domain and the transmembrane domain.

In some aspects, the CAR contains an extracellular binding domain (e.g., CD19 binding domain and a transmembrane domain that contains a transmembrane portion of CD28). The extracellular domain and transmembrane can be linked directly or indirectly. In some embodiments, the extracellular domain and transmembrane are linked by a spacer, such as any described herein. In some embodiments, the chimeric antigen receptor contains an intracellular domain containing a CD3zeta intracellular signaling domain and a signaling domain of a T cell costimulatory molecule. In some embodiments, the costimulatory signaling domain is between the transmembrane domain and CD3zeta intracellular signaling domain. In some aspects, the T cell costimulatory molecule is 4-1BB.

Also provided herein are polynucleotides encoding any of the provided recombinant receptors, such as any of the provided CARs.

In some embodiments, the CARs are encoded by polynucleotides. The provided polynucleotides can be incorporated into constructs, such as deoxyribonucleic acid (DNA) or RNA constructs, such as those that can be introduced into cells for expression of the encoded CAR. Hence, also provided herein are engineered cells containing any of the provided CARs. Exemplary engineered cells and methods of preparing same are described in Section VI. Also provided herein are compositions and articles of manufacture and uses of any of the engineered cells. Also provided are cells expressing the recombinant receptors and uses thereof in adoptive cell therapy, such as treatment of diseases and disorders associated with expression of the antigen, such as CD19 expression.

### A. Extracellular Antigen-Binding Domains

The extracellular binding domain of a CAR provides a means for binding to an antigen or antigens. In some embodiments, the CAR includes an extracellular binding domain that is an antigen-binding portion or portions of an antibody molecule. In some embodiments, the antigen-binding domain is a portion of an antibody molecule, generally including a variable heavy (V_{H}) chain region and/or variable light (V_{L}) chain region of the antibody. In some embodiments, the CAR includes an antigen-binding portion or portions of an antibody molecule, such as a single-chain antibody fragment (scFv) derived from the variable heavy (V_{H}) and variable light (V_{L}) chains of a monoclonal antibody (mAb). In some embodiments, the antigen-binding domain is a single domain antibody (sdAb), such as sdFv, nanobody, V_{H}H and V_{NAR}. In some embodiments, an antigen-binding fragment comprises antibody variable regions joined by a flexible linker.

In some embodiments, the extracellular binding domain of the CAR provides a means for binding to CD19. In some embodiments, the extracellular binding domain is an antibody or an antigen-binding fragment (e.g., scFv or V_{H} domain) that specifically recognizes an antigen, such as CD19. In some embodiments, the extracellular binding domain includes antibody variable chain sequences (e.g., variable heavy chain and variable light chain sequences) that specifically recognize CD19. In some embodiments, the antibody or antigen-binding fragment, such as including antibody variable chain sequences, is derived from, or is a variant of, antibodies or antigen-binding fragment that specifically binds to CD19. In some embodiments, the antibody or an antigen-binding fragment (e.g., scFv) contains a variable heavy chain and a variable light chain with six CDRs, CDRH1-3 and CDRL1-3, that confer binding to CD19.

The terms “complementarity determining region,” and “CDR,” synonymous with “hypervariable region” or “HVR,” are known to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4).

Table 4, below, exemplifies exemplary numbering and lists exemplary position boundaries of CDR-L1, CDR-L2, CDR-L3 and CDR-H1, CDR-H2, CDR-H3 as identified by Kabat, Chothia, AbM, and Contact schemes, respectively. For CDR-H1, residue numbering is listed using both the Kabat and Chothia numbering schemes. FRs are located between CDRs, for example, with FR-Li located before CDR-L1, FR-L2 located between CDR-L1 and CDR-L2, FR-L3 located between CDR-L2 and CDR-L3 and so forth. It is noted that because the shown Kabat numbering scheme places insertions at H35A and H35B, the end of the Chothia CDR-H1 loop when numbered using the shown Kabat numbering convention varies between H32 and H34, depending on the length of the loop.

TABLE 4
Boundaries of CDRs according to various numbering schemes.

| CDR | Kabat | Chothia | AbM | Contact |
|---|---|---|---|---|
| CDR-L1 | L24--L34 | L24--L34 | L24--L34 | L30--L36 |
| CDR-L2 | L50--L56 | L50--L56 | L50--L56 | L46--L55 |
| CDR-L3 | L89--L97 | L89--L97 | L89--L97 | L89--L96 |
| CDR-H1 | H31--H35B | H26--H32 . . . 34 | H26--H35B | H30--H35B |
| (Kabat Numbering¹) | | | | |
| CDR-H1 | H31--H35 | H26--H32 | H26--H35 | H30--H35 |
| (Chothia Numbering²) | | | | |
| CDR-H2 | H50--H65 | H52--H56 | H50--H58 | H47--H58 |
| CDR-H3 | H95--H102 | H95--H102 | H95--H102 | H93--H101 |

¹Kabat et al. (1991), “Sequences of Proteins of Immunological Interest,” 5th Ed. Public Health Service, National Institutes of Health, Bethesda, MD
²Al-Lazikani et al., J Mol Biol., 1997; 273(4): 927-48).

Thus, unless otherwise specified, a “CDR” or “complementary determining region,” or individual specified CDRs (e.g., CDR-H1, CDR-H2, CDR-H3), of a given antibody or region thereof, such as a variable region thereof, should be understood to encompass a (or the specific) complementary determining region as defined by any of the aforementioned schemes, or other known schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes, or other known schemes. In some embodiments, where it is stated that an antibody or antigen-binding fragment thereof comprises a CDR-H1, a CDR-H2, and a CDR-H3 as contained within a given V_{H} region amino acid sequence and a CDR-L1, a CDR-L2, and a CDR-L3 as contained within a given V_{L} region amino acid sequence, the CDRs can be defined by any of the aforementioned schemes, such as Kabat, Chothia, AbM, IgBLAST, IMGT, or Contact method, or other known scheme. In some embodiments, specific CDR sequences are specified. Exemplary CDR sequences of provided antibodies are described using various numbering schemes, although it is understood that a provided antibody can include CDRs as described according to any of the other aforementioned numbering schemes or other known numbering schemes.

Likewise, unless otherwise specified, a FR or individual specified FR(s) (e.g., FR-H1, FR-H2, FR-H3, FR-H4, FR-L1, FR-L2, FR-L3, and/or FR-L4), of a given antibody or region thereof, such as a variable region thereof, should be understood to encompass a (or the specific) framework region as defined by any of the known schemes. In some instances, the scheme for identification of a particular CDR, FR, or FRs or CDRs is specified, such as the CDR as defined by the Kabat, Chothia, AbM, IgBLAST, IMGT, or Contact method, or other known schemes. In other cases, the particular amino acid sequence of a CDR or FR is given. In some embodiments, where it is stated that an antibody or antigen-binding fragment thereof comprises a FR-H1, a FR-H2, a FR-H3, and a FR-H4 as contained within a given V_{H} region amino acid sequence and a FR-Li, a FR-L2, a FR-L3, and a FR-L4 as contained within a given V_{L} region amino acid sequence, the FRs can be defined by any of the aforementioned schemes, such as Kabat, Chothia, AbM, IgBLAST, IMGT, or Contact method, or other known scheme.

The term “variable region” or “variable domain” refers to the domain of an antibody heavy or light chain that is involved in binding the antibody to antigen. The variable regions of the heavy chain and light chain (V_{H} and V_{L}, respectively) of a native antibody generally have similar structures, with each domain comprising four conserved framework regions (FRs) and three CDRs (See, e.g., Kindt et al. Kuby Immunology, 6th ed., W.H. Freeman and Co., page 91 (2007)). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

In some embodiments, the antibody or antigen-binding fragment thereof, in the provided CAR, is a single-chain antibody fragment, such as a single chain variable fragment (scFv) or a diabody or a single domain antibody (sdAb). In some embodiments, the antibody or antigen-binding fragment is a single domain antibody comprising only the V_{H} region. In some embodiments, the antibody or antigen binding fragment is an scFv comprising a heavy chain variable (V_{H}) region and a light chain variable (V_{L}) region. In some embodiments, the single-chain antibody fragment (e.g., scFv) includes one or more linkers joining two antibody domains or regions, such as a heavy chain variable (V_{H}) region and a light chain variable (V_{L}) region. The linker typically is a peptide linker, e.g., a flexible and/or soluble peptide linker. Among the linkers are those rich in glycine and serine and/or in some cases threonine. In some embodiments, the linkers further include charged residues such as lysine and/or glutamate, which can improve solubility. In some embodiments, the linkers further include one or more proline.

The linker typically is a peptide linker, e.g., a flexible and/or soluble peptide linker, such as one rich in glycine and serine. In some embodiments, the antigen binding domain comprises a linker between the V_{H} and V_{L} regions. In some embodiments, in order from N- to C-terminus, the antigen binding domain comprises one of the V_{H} and V_{L} regions, a linker, and the other of the V_{H} and V_{L} regions. In some aspects, the linkers rich in glycine and serine (and/or threonine) include at least 80%, 85%, 90%, 91%, 92%, 93%, 94%, 95%, 96%, 97%, 98%, or 99% such amino acid(s). In some embodiments, they include at least at or about 50%, 55%, 60%, 70%, or 75%, glycine, serine, and/or threonine. In some embodiments, the linker is comprised substantially entirely of glycine, serine, and/or threonine. The linkers generally are between about 5 and about 50 amino acids in length, typically between at or about 10 and at or about 30, e.g., 10, 11, 12, 13, 14, 15, 16, 17, 18, 19, 20, 21, 22, 23, 24, 25, 26, 27, 28, 29, or 30, and in some examples between 10 and 25 amino acids in length. Exemplary linkers include linkers having various numbers of repeats of the sequence GGGGS (4GS; SEQ ID NO: 20), such as between 2, 3, 4 and 5 repeats of such a sequence. Exemplary linkers include those having or consisting of an sequence set forth in SEQ ID NO: 18 (GGGGSGGGGSGGGGS). Exemplary linkers further include those having or consisting of the sequence set forth in SEQ ID NO: 19 (GSTSGSGKPGSGEGSTKG), SEQ ID NO: 21 (GGGGSGGGGS), and SEQ ID NO: 22 (GGGGSGGGGSGGGGSGGGGS). In some embodiments, the linker is set forth in SEQ ID NO: 18.

Accordingly, in some embodiments, the provided embodiments include single-chain antibody fragments, e.g., scFvs, comprising one or more of the aforementioned linkers, such as glycine/serine rich linkers, including linkers having repeats of GGGS (SEQ ID NO: 20), such as the linker set forth in SEQ ID NO: 18, SEQ ID NO: 21, or SEQ ID NO: 22.

In some embodiments, the CAR includes a CD19-binding domain comprising an antibody, such as a heavy chain variable (V_{H}) region and/or light chain variable (V_{L}) region of the antibody. In some embodiments, the (V_{H}) region and the (V_{L}) region of the CD19-binding domain are joined by a linker. In some embodiments, the (V_{H}) region and the (V_{L}) region of the CD19-binding domain comprise an scFv antibody fragment. In some embodiments, the provided CD19-binding CARs contain an antibody, such as an anti-CD19 antibody, or an antigen-binding fragment thereof that confers the CD19-binding properties of the provided CAR.

In some embodiments, the antibody, e.g., the anti-CD19 antibody, or antigen-binding fragment, contains a heavy and/or light chain variable (V_{H} or V_{L}) region sequence as described, or a sufficient antigen-binding portion thereof. In some embodiments, the anti-CD19 antibody, e.g., antigen-binding fragment, contains a V_{H} region sequence or sufficient antigen-binding portion thereof that contains a CDR-H1, CDR-H2 and/or CDR-H3 as described. In some embodiments, the anti-CD19 antibody, e.g., antigen-binding fragment, contains a V_{L} region sequence or sufficient antigen-binding portion that contains a CDR-L1, CDR-L2 and/or CDR-L3 as described. In some embodiments, the anti-CD19 antibody, e.g., antigen-binding fragment, contains a V_{H} region sequence that contains a CDR-H1, CDR-H2 and/or CDR-H3 as described and contains a V_{L} region sequence that contains a CDR-L1, CDR-L2 and/or CDR-L3 as described. Also among the antibodies are those having sequences at least at or about 90%, at or about 91%, at or about 92%, at or about 93%, at or about 94%, at or about 95%, at or about 96%, at or about 97%, at or about 98%, or at or about 99% identical to such a sequence.

In some embodiments, the VH and VL region of the anti-CD19 antibody in a provided CAR is the VH and VL sequence of a CAR T cell therapy that targets CD19. Exemplary CAR T cell therapies that target CD19 include those investigated or being investigated in clinical trials NCT02644655, NCT03744676, NCT01087294, NCT03366350, NCT03790891, NCT03497533, NCT04007029, NCT03960840, NCT04049383, NCT04094766, NCT03366324, NCT02546739, NCT03448393, NCT03467256, NCT03488160, NCT04012879, NCT03016377, NCT03468153, NCT03483688, NCT03398967, NCT03229876, NCT03455972, NCT03423706, NCT03497533, and NCT04002401, including FDA-approved products BREYANZI® (lisocabtagene maraleucel), TECARTUS™ (brexucabtagene autoleucel), KYMRIAH™ (tisagenlecleucel), and YESCARTA™ (axicabtagene ciloleucel).

In some embodiments, the V_{H} and a V_{L} is derived from an antibody or an antibody fragment specific to CD19. In some embodiments, the antibody or antibody fragment that binds CD19 is a mouse derived antibody such as FMC63 and SJ25C1. In some embodiments, exemplary antibody or antibody fragment include human anti-CD19 antibodies, such as those described in U.S. Patent Publication No. WO 2014/031687, US 2016/0152723 and WO 2016/033570, the contents of each of which are incorporated by reference in their entirety.

In some embodiments the antigen-binding domain includes a V_{H} and/or V_{L} derived from FMC63, which, in some aspects, can be an scFv. In some embodiments the scFv and/or V_{H} domains is derived from FMC63. FMC63 generally refers to a mouse monoclonal IgG1 antibody raised against Nalm-1 and -16 cells expressing CD19 of human origin (Ling, N. R., et al. (1987). Leucocyte typing III. 302). The FMC63 antibody comprises CDRH1 and H2 set forth in SEQ ID NOs: 3 and 4 respectively, and CDRH3 set forth in SEQ ID NOs: 5 or 6, and CDRL1 set forth in SEQ ID NO: 7 and CDRL2 set forth in SEQ ID NOs: 8, or 9, and CDRL3 sequences set forth in SEQ ID NOs: 10, or 11. The FMC63 antibody comprises the heavy chain variable region (V_{H}) comprising the amino acid sequence of SEQ ID NO: 1 and the light chain variable region (V_{L}) comprising the amino acid sequence of SEQ ID NO: 2. In some embodiments, the scFv comprises a variable light chain containing the CDRL1 sequence of SEQ ID NO: 7, a CDRL2 sequence of SEQ ID NO: 8, and a CDRL3 sequence of SEQ ID NO: 10 and/or a variable heavy chain containing a CDRH1 sequence of SEQ ID NO: 3, a CDRH2 sequence of SEQ ID NO: 4, and a CDRH3 sequence of SEQ ID NO: 5. In some embodiments, the scFv comprises a variable heavy chain region of FMC63 set forth in SEQ ID NO: 1 and a variable light chain region of FMC63 set forth in SEQ ID NO: 2.

In some embodiments, the anti-CD19 binding domain of the CAR includes the V_{H} and V_{L} sequences of the antigen-binding domain of the anti-CD19 CAR of BREYANZI® (lisocabtagene maraleucel).

In some embodiments, the anti-CD19 binding domain of the CAR includes the V_{H} and V_{L} sequences of the antigen-binding domain of the anti-CD19 CAR of TECARTUS™ (brexucabtagene autoleucel).

In some embodiments, the anti-CD19 binding domain of the CAR includes the V_{H} and V_{L} sequences of the antigen-binding domain of the anti-CD19 CAR of KYMRIAH™ (tisagenlecleucel).

In some embodiments, the anti-CD19 binding domain of the CAR includes the V_{H} and V_{L} sequences of the antigen-binding domain of the anti-CD19 CAR of YESCARTA™ (axicabtagene ciloleucel).

In some embodiments the anti-CD19 antigen-binding domain of the provided CAR includes a V_{H} and/or V_{L} derived from SJ25C1, which, in some aspects, can be an scFv. SJ25C1 is a mouse monoclonal IgG1 antibody raised against Nalm-1 and -16 cells expressing CD19 of human origin (Ling, N. R., et al. (1987). Leucocyte typing III. 302). The SJ25C1 antibody comprises CDRH1, H2 and H3 set forth in SEQ ID NOs: 71-73, respectively, and CDRL1, L2 and L3 sequences set forth in SEQ ID NOs: 68-70, respectively. The SJ25C1 antibody comprises the heavy chain variable region (V_{H}) comprising the amino acid sequence of SEQ ID NO: 74 and the light chain variable region (V_{L}) comprising the amino acid sequence of SEQ ID NO: 75. In some embodiments, the scFv comprises a variable light chain containing the CDRL1 sequence of SEQ ID NO: 68, a CDRL2 sequence of SEQ ID NO: 69, and a CDRL3 sequence of SEQ ID NO: 70 and/or a variable heavy chain containing a CDRH1 sequence of SEQ ID NO: 71, a CDRH2 sequence of SEQ ID NO: 72, and a CDRH3 sequence of SEQ ID NO: 73. In some embodiments, the scFv comprises a variable heavy chain region of SJ25C1 set forth in SEQ ID NO: 74 and a variable light chain region of SJ25C1 set forth in SEQ ID NO: 75.

In some embodiments, the CAR contains an antibody or antigen-binding fragment thereof, that has a heavy chain variable (V_{H}) region having the amino acid sequence set forth in SEQ ID NO: 1 or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the V_{H} region amino acid set forth in SEQ ID NO: 1 or contains a CDR-H1, CDR-H2, and/or CDR-H3 present in such a V_{H} sequence.

In some embodiments, the CAR contains an antibody or antigen-binding fragment thereof, that has a heavy chain variable (V_{H}) region having the amino acid sequence set forth in SEQ ID NO: 23 or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the V_{H} region amino acid set forth in SEQ ID NO: 23 or contains a CDR-H1, CDR-H2, and/or CDR-H3 present in such a V_{H} sequence.

In some embodiments, the CAR contains an antibody or antigen-binding fragment thereof, that has a heavy chain variable (V_{H}) region having the amino acid sequence set forth in SEQ ID NO: 24 or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the V_{H} region amino acid set forth in SEQ ID NO: 24 or contains a CDR-H1, CDR-H2, and/or CDR-H3 present in such a V_{H} sequence.

In some embodiments, the V_{H} region of an antibody or antigen-binding fragment thereof comprises a CDR-H1, CDR-H2, and/or CDR-H3 according to Kabat numbering. In some embodiments, the V_{H} region of an antibody or antigen-binding fragment thereof comprises a CDR-H1, CDR-H2, and/or CDR-H3 according to Chothia numbering. In some embodiments, the V_{H} region of an antibody or antigen-binding fragment thereof comprises a CDR-H1, CDR-H2, and/or CDR-H3 according to AbM numbering.

In some embodiments, the CAR contains an antibody or antigen-binding fragment thereof, that has a variable heavy chain (V_{H}) region comprising a CDR-H1 comprising the amino acid sequence set forth in SEQ ID NO: 3, a CDR-H2 comprising the amino acid sequence set forth in SEQ ID NO: 4, and a CDR-H3 comprising the amino acid sequence set forth in SEQ ID NO: 5 or 6. In some embodiments, the antibody or antigen-binding fragment thereof comprises a V_{H} region comprising a CDR-H1, a CDR-H2, and a CDR-H3 comprising the amino acid sequence set forth in SEQ ID NO: 3, 4, and 5, respectively or 3, 4, and 6 respectively. In some embodiments, the antibody or antigen-binding fragment thereof comprises a V_{H} region comprising the amino acid sequence set forth in SEQ ID NO: 3, 4, and 5. In some embodiments, the antibody or antigen-binding fragment thereof comprises a V_{H} region comprising the amino acid sequence set forth in SEQ ID NO: 3, 4, and 6. In some embodiments, the antibody or antigen-binding fragment thereof comprises a CDR-H1, CDR-H2 and CDR-H3, respectively, comprising the amino acid sequence of a CDR-H1, a CDR-H2, and a CDR-H3 contained within the V_{H} region amino acid sequence set forth in SEQ ID NO: 1.

In some embodiments of the antibody or antigen-binding fragment thereof provided herein, the V_{H} region comprises any of the CDR-H1, CDR-H2 and CDR-H3 as described and comprises a framework region 1 (FR1), a FR2, a FR3 and/or a FR4 having at least at or about 90%, at or about 91%, at or about 92%, at or about 93%, at or about 94%, at or about 95%, at or about 96%, at or about 97%, at or about 98%, or at or about 99% sequence identity, respectively, to a FR1, a FR2, a FR3 and/or a FR4 contained within the V_{H} region amino acid sequence set forth in SEQ ID NO: 1. In some embodiments, the antibody or antigen-binding fragment thereof comprises a V_{H} region comprising the amino acid sequence set forth in SEQ ID NO: 1.

In some embodiments, the CAR contains an antibody or antigen-binding fragment thereof, that has a variable heavy chain (V_{H}) region comprising a CDR-H1 comprising the amino acid sequence set forth in SEQ ID NO: 35, a CDR-H2 comprising the amino acid sequence set forth in SEQ ID NO: 36, and a CDR-H3 comprising the amino acid sequence set forth in SEQ ID NO: 37. In some embodiments, the antibody or antigen-binding fragment thereof comprises a V_{H} region comprising a CDR-H1, a CDR-H2, and a CDR-H3 comprising the amino acid sequence set forth in SEQ ID NO: 35, 36, and 37, respectively. In some embodiments, the antibody or antigen-binding fragment thereof comprises a V_{H} region comprising the amino acid sequence set forth in SEQ ID NO: 35, 36, and 37. In some embodiments, the antibody or antigen-binding fragment thereof comprises a CDR-H1, CDR-H2 and CDR-H3, respectively, comprising the amino acid sequence of a CDR-H1, a CDR-H2, and a CDR-H3 contained within the V_{H} region amino acid sequence set forth in SEQ ID NO: 23.

In some embodiments of the antibody or antigen-binding fragment thereof provided herein, the V_{H} region comprises any of the CDR-H1, CDR-H2 and CDR-H3 as described and comprises a framework region 1 (FR1), a FR2, a FR3 and/or a FR4 having at least at or about 90%, at or about 91%, at or about 92%, at or about 93%, at or about 94%, at or about 95%, at or about 96%, at or about 97%, at or about 98%, or at or about 99% sequence identity, respectively, to a FR1, a FR2, a FR3 and/or a FR4 contained within the V_{H} region amino acid sequence set forth in SEQ ID NO: 23. In some embodiments, the antibody or antigen-binding fragment thereof comprises a V_{H} region comprising the amino acid sequence set forth in SEQ ID NO: 23.

In some embodiments, the CAR contains an antibody or antigen-binding fragment thereof, that has a variable heavy chain (V_{H}) region comprising a CDR-H1 comprising the amino acid sequence set forth in SEQ ID NO: 35, a CDR-H2 comprising the amino acid sequence set forth in SEQ ID NO: 36, and a CDR-H3 comprising the amino acid sequence set forth in SEQ ID NO: 37. In some embodiments, the antibody or antigen-binding fragment thereof comprises a V_{H} region comprising a CDR-H1, a CDR-H2, and a CDR-H3 comprising the amino acid sequence set forth in SEQ ID NO: 35, 36, and 37, respectively. In some embodiments, the antibody or antigen-binding fragment thereof comprises a V_{H} region comprising the amino acid sequence set forth in SEQ ID NO: 35, 36, and 37. In some embodiments, the antibody or antigen-binding fragment thereof comprises a CDR-H1, CDR-H2 and CDR-H3, respectively, comprising the amino acid sequence of a CDR-H1, a CDR-H2, and a CDR-H3 contained within the V_{H} region amino acid sequence set forth in SEQ ID NO: 24.

In some embodiments of the antibody or antigen-binding fragment thereof provided herein, the V_{H} region comprises any of the CDR-H1, CDR-H2 and CDR-H3 as described and comprises a framework region 1 (FR1), a FR2, a FR3 and/or a FR4 having at least at or about 90%, at or about 91%, at or about 92%, at or about 93%, at or about 94%, at or about 95%, at or about 96%, at or about 97%, at or about 98%, or at or about 99% sequence identity, respectively, to a FR1, a FR2, a FR3 and/or a FR4 contained within the V_{H} region amino acid sequence set forth in SEQ ID NO: 24. In some embodiments, the antibody or antigen-binding fragment thereof comprises a V_{H} region comprising the amino acid sequence set forth in SEQ ID NO: 24.

In some embodiments, the antibody or antibody fragment, in the provided CAR comprising a V_{H} region further comprises a light chain or a sufficient antigen binding portion thereof. For example, in some embodiments, the antibody or antigen-binding fragment thereof contains a V_{H} region and a V_{L} region, or a sufficient antigen-binding portion of a V_{H} and V_{L} region. In such embodiments, a V_{H} region sequence can be any of the above described V_{H} sequence. In some such embodiments, the antibody is an antigen-binding fragment, such as a Fab or an scFv. In some such embodiments, the antibody is a full-length antibody that also contains a constant region.

In some embodiments, a CAR provided herein, contains an antibody such as an anti-CD19 antibody, or antigen-binding fragment thereof that contains any of the above V_{H} region and contains a variable light chain region or a sufficient antigen binding portion thereof. For example, in some embodiments, the CAR contains an antibody or antigen-binding fragment thereof that contains a V_{H} region and a variable light chain (V_{L}) region, or a sufficient antigen-binding portion of a V_{H} and V_{L} region. In such embodiments, a V_{H} region sequence can be any of the above described V_{H} sequence. In some such embodiments, the antibody is an antigen-binding fragment, such as a Fab or an scFv. In some such embodiments, the antibody is a full-length antibody that also contains a constant region.

In some embodiments, the CAR contains an antibody or antigen-binding fragment thereof, that has a light chain variable (V_{L}) region having the amino acid sequence set forth in SEQ ID NO: 2, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the V_{L} region amino acid set forth in SEQ ID NO: 2, or contains a CDR-L1, CDR-L2, and/or CDR-L3 present in such a V_{L} sequence.

In some embodiments, the CAR contains an antibody or antigen-binding fragment thereof, that has a light chain variable (V_{L}) region having the amino acid sequence set forth in SEQ ID NO: 25, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the V_{L} region amino acid set forth in SEQ ID NO: 25, or contains a CDR-L1, CDR-L2, and/or CDR-L3 present in such a V_{L} sequence.

In some embodiments, the CAR contains an antibody or antigen-binding fragment thereof, that has a light chain variable (V_{L}) region having the amino acid sequence set forth in SEQ ID NO: 26, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the V_{L} region amino acid set forth in SEQ ID NO: 26, or contains a CDR-L1, CDR-L2, and/or CDR-L3 present in such a V_{L} sequence.

In some embodiments, the CAR contains an antibody or antigen-binding fragment thereof, that has a light chain variable (V_{L}) region having the amino acid sequence set forth in SEQ ID NO: 27, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the V_{L} region amino acid set forth in SEQ ID NO: 27, or contains a CDR-L1, CDR-L2, and/or CDR-L3 present in such a V_{L} sequence.

In some embodiments, the CAR contains an antibody or antigen-binding fragment thereof, that has a light chain variable (V_{L}) region having the amino acid sequence set forth in SEQ ID NO: 28, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the V_{L} region amino acid set forth in SEQ ID NO: 28, or contains a CDR-L1, CDR-L2, and/or CDR-L3 present in such a V_{L} sequence.

In some embodiments, the CAR contains an antibody or antigen-binding fragment thereof, that has a light chain variable (V_{L}) region having the amino acid sequence set forth in SEQ ID NO: 29, or an amino acid sequence that has at least at or about 90%, at or about 91%, at or about 92%, at or about 93%, at or about 94%, at or about 95%, at or about 96%, at or about 97%, at or about 98%, or at or about 99% sequence identity to the V_{L} region amino acid set forth in SEQ ID NO: 29, or contains a CDR-L1, CDR-L2, and/or CDR-L3 present in such a V_{L} sequence.

In some embodiments, the V_{L} region of an antibody or antigen-binding fragment thereof comprises a CDR-L1, CDR-L2, and/or CDR-L3 according to Kabat numbering. In some embodiments, the V_{L} region of an antibody or antigen-binding fragment thereof comprises a CDR-L1, CDR-L2, and/or CDR-L3 according to Chothia numbering. In some embodiments, the V_{L} region of an antibody or antigen-binding fragment thereof comprises a CDR-L1, CDR-L2, and/or CDR-L3 according to AbM numbering.

In some embodiments, the CAR contains an antibody or antigen-binding fragment thereof, that has a variable light chain (V_{L}) region comprising a CDR-L1 comprising the amino acid sequence set forth in SEQ ID NO: 7, a CDR-L2 comprising the amino acid sequence set forth in SEQ ID NO: 8 or 9, and a CDR-L3 comprising the amino acid sequence set forth in SEQ ID NO: 10 or 11. In some embodiments, the antibody or antigen-binding fragment thereof comprises a V_{L} region comprising a CDR-L1, a CDR-L2, and a CDR-L3 comprising the amino acid sequence set forth in SEQ ID NO: 7, 8, and 10, respectively; 7, 9, and 10, respectively; 7, 8, and 11, respectively; or 7, 9, and 11, respectively.

In some embodiments, the antibody or antigen-binding fragment thereof contains a CDR-L1, CDR-L2, and CDR-L3, respectively, contained within the V_{L} region amino acid sequence set forth in SEQ ID NO: 2.

In some embodiments, the CAR contains an antibody or antigen-binding fragment thereof, that has a variable light chain (V_{L}) region comprising a CDR-L1 comprising the amino acid sequence set forth in SEQ ID NO: 38, a CDR-L2 comprising the amino acid sequence set forth in SEQ ID NO: 39, and a CDR-L3 comprising the amino acid sequence set forth in SEQ ID NO: 40. In some embodiments, the antibody or antigen-binding fragment thereof comprises a V_{L} region comprising a CDR-L1, a CDR-L2, and a CDR-L3 comprising the amino acid sequence set forth in SEQ ID NO: 38, 39, and 40, respectively. In some embodiments, the antibody or antigen-binding fragment thereof contains a CDR-L1, CDR-L2, and CDR-L3, respectively, contained within the V_{L} region amino acid sequence set forth in SEQ ID NO: 25.

In some embodiments, the CAR contains an antibody or antigen-binding fragment thereof, that has a variable light chain (V_{L}) region comprising a CDR-L1 comprising the amino acid sequence set forth in SEQ ID NO: 41, a CDR-L2 comprising the amino acid sequence set forth in SEQ ID NO: 42, and a CDR-L3 comprising the amino acid sequence set forth in SEQ ID NO: 43. In some embodiments, the antibody or antigen-binding fragment thereof comprises a V_{L} region comprising a CDR-L1, a CDR-L2, and a CDR-L3 comprising the amino acid sequence set forth in SEQ ID NO: 41, 42, and 43, respectively. In some embodiments, the antibody or antigen-binding fragment thereof contains a CDR-L1, CDR-L2, and CDR-L3, respectively, contained within the V_{L} region amino acid sequence set forth in SEQ ID NO: 26.

In some embodiments, the CAR contains an antibody or antigen-binding fragment thereof, that has a variable light chain (V_{L}) region comprising a CDR-L1 comprising the amino acid sequence set forth in SEQ ID NO: 44, a CDR-L2 comprising the amino acid sequence set forth in SEQ ID NO: 45, and a CDR-L3 comprising the amino acid sequence set forth in SEQ ID NO: 46. In some embodiments, the antibody or antigen-binding fragment thereof comprises a V_{L} region comprising a CDR-L1, a CDR-L2, and a CDR-L3 comprising the amino acid sequence set forth in SEQ ID NO: 44, 45, and 46, respectively. In some embodiments, the antibody or antigen-binding fragment thereof contains a CDR-L1, CDR-L2, and CDR-L3, respectively, contained within the V_{L} region amino acid sequence set forth in SEQ ID NO: 27.

In some embodiments, the CAR contains an antibody or antigen-binding fragment thereof, that has a variable light chain (V_{L}) region comprising a CDR-L1 comprising the amino acid sequence set forth in SEQ ID NO: 44, a CDR-L2 comprising the amino acid sequence set forth in SEQ ID NO: 45, and a CDR-L3 comprising the amino acid sequence set forth in SEQ ID NO: 47. In some embodiments, the antibody or antigen-binding fragment thereof comprises a V_{L} region comprising a CDR-L1, a CDR-L2, and a CDR-L3 comprising the amino acid sequence set forth in SEQ ID NO: 44, 45, and 47, respectively. In some embodiments, the antibody or antigen-binding fragment thereof contains a CDR-L1, CDR-L2, and CDR-L3, respectively, contained within the V_{L} region amino acid sequence set forth in SEQ ID NO: 28.

In some embodiments, the CAR contains an antibody or antigen-binding fragment thereof, that has a variable light chain (V_{L}) region comprising a CDR-L1 comprising the amino acid sequence set forth in SEQ ID NO: 48, a CDR-L2 comprising the amino acid sequence set forth in SEQ ID NO: 49, and a CDR-L3 comprising the amino acid sequence set forth in SEQ ID NO: 50. In some embodiments, the antibody or antigen-binding fragment thereof comprises a V_{L} region comprising a CDR-L1, a CDR-L2, and a CDR-L3 comprising the amino acid sequence set forth in SEQ ID NO: 48, 49, and 50, respectively. In some embodiments, the antibody or antigen-binding fragment thereof contains a CDR-L1, CDR-L2, and CDR-L3, respectively, contained within the V_{L} region amino acid sequence set forth in SEQ ID NO: 29.

Among the CARs provided herein is a CAR in which the antibody, such as an anti-CD19 antibody, or antibody fragment, in the provided CAR, comprises a V_{H} region amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 1 and a V_{L} region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 2.

In some embodiments, the V_{H} region of the antibody or antigen-binding fragment thereof comprises a CDR-H1, a CDR-H2, a CDR-H3, respectively, comprising the amino acid sequences of CDR-H1, CDR-H2, and CDR-H3 contained within the V_{H} region amino acid sequence set forth in SEQ ID NO: 1; and comprises a CDR-L1, a CDR-L2, a CDR-L3, respectively, comprising the amino acid sequences of CDR-L1, CDR-L2, and CDR-L3, respectively contained within the V_{L} region amino acid sequence set forth in SEQ ID NO: 2.

In some embodiments, the V_{H} region of the antibody or antigen-binding fragment thereof comprise the amino acid sequence set forth in SEQ ID NO: 1 and the V_{L} region of the antibody or antigen-binding fragment comprises the amino acid sequence set forth in SEQ ID NO: 2. In some embodiments, the V_{H} and V_{L} regions of the antibody or antigen-binding fragment thereof comprise the amino acid sequences set forth in SEQ ID NOs: 1 and 2, respectively, or any antibody or antigen-binding fragment thereof that has at least 90% sequence identity to any of the above V_{H} and V_{L}, such as at least 90%, 91%, 92%, 93%, 94%, 95%, 96%, 97%, 98%, or 99% sequence identity thereto.

For example, the V_{H} and V_{L} regions of the antibody or antigen-binding fragment thereof provided therein comprise the amino acid sequence set forth in SEQ ID NO: 1 and 2, respectively.

Among the CARs provided herein is a CAR in which the antibody, such as an anti-CD19 antibody, or antibody fragment, in the provided CAR, comprises a V_{H} region amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 23, and a V_{L} region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 25.

In some embodiments, the V_{H} region of the antibody or antigen-binding fragment thereof comprises a CDR-H1, a CDR-H2, a CDR-H3, respectively, comprising the amino acid sequences of CDR-H1, CDR-H2, and CDR-H3 contained within the V_{H} region amino acid sequence set forth in SEQ ID NO: 23; and comprises a CDR-L1, a CDR-L2, a CDR-L3, respectively, comprising the amino acid sequences of CDR-L1, CDR-L2, and CDR-L3, respectively contained within the V_{L} region amino acid sequence set forth in SEQ ID NO: 25.

In some embodiments, the V_{H} region of the antibody or antigen-binding fragment thereof comprise the amino acid sequence set forth in SEQ ID NO: 23 and the V_{L} region of the antibody or antigen-binding fragment comprises the amino acid sequence set forth in SEQ ID NO: 25. In some embodiments, the V_{H} and V_{L} regions of the antibody or antigen-binding fragment thereof comprise the amino acid sequences set forth in SEQ ID NO: 23 and 25, respectively, or any antibody or antigen-binding fragment thereof that has at least 90% sequence identity to any of the above V_{H} and V_{L}, such as at least 90%, 91%, 92%, 93%, 94%, 95%, 96%, 97%, 98%, or 99% sequence identity thereto.

For example, the V_{H} and V_{L} regions of the antibody or antigen-binding fragment thereof provided therein comprise the amino acid sequence set forth in SEQ ID NO: 23 and 25, respectively.

Among the CARs provided herein is a CAR in which the antibody, such as an anti-CD19 antibody, or antibody fragment, in the provided CAR, comprises a V_{H} region amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NOs: 23, and a V_{L} region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 26.

In some embodiments, the V_{H} region of the antibody or antigen-binding fragment thereof comprises a CDR-H1, a CDR-H2, a CDR-H3, respectively, comprising the amino acid sequences of CDR-H1, CDR-H2, and CDR-H3 contained within the V_{H} region amino acid sequence set forth in SEQ ID NO: 23; and comprises a CDR-L1, a CDR-L2, a CDR-L3, respectively, comprising the amino acid sequences of CDR-L1, CDR-L2, and CDR-L3, respectively contained within the V_{L} region amino acid sequence set forth in SEQ ID NO: 26.

In some embodiments, the V_{H} region of the antibody or antigen-binding fragment thereof comprise the amino acid sequence set forth in SEQ ID NO: 23 and the V_{L} region of the antibody or antigen-binding fragment comprises the amino acid sequence set forth in SEQ ID NO: 26. In some embodiments, the V_{H} and V_{L} regions of the antibody or antigen-binding fragment thereof comprise the amino acid sequences set forth in SEQ ID NO: 23 and 26, respectively, or any antibody or antigen-binding fragment thereof that has at least 90% sequence identity to any of the above V_{H} and V_{L}, such as at least 90%, 91%, 92%, 93%, 94%, 95%, 96%, 97%, 98%, or 99% sequence identity thereto.

For example, the V_{H} and V_{L} regions of the antibody or antigen-binding fragment thereof provided therein comprise the amino acid sequence set forth in SEQ ID NO: 23 and 26, respectively.

Among the CARs provided herein is a CAR in which the antibody, such as an anti-CD19 antibody, or antibody fragment, in the provided CAR, comprises a V_{H} region amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 24, and a V_{L} region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 27.

In some embodiments, the V_{H} region of the antibody or antigen-binding fragment thereof comprises a CDR-H1, a CDR-H2, a CDR-H3, respectively, comprising the amino acid sequences of CDR-H1, CDR-H2, and CDR-H3 contained within the V_{H} region amino acid sequence set forth in SEQ ID NO: 24; and comprises a CDR-L1, a CDR-L2, a CDR-L3, respectively, comprising the amino acid sequences of CDR-L1, CDR-L2, and CDR-L3, respectively contained within the V_{L} region amino acid sequence set forth in SEQ ID NO: 27.

In some embodiments, the V_{H} region of the antibody or antigen-binding fragment thereof comprise the amino acid sequence set forth in SEQ ID NO: 24 and the V_{L} region of the antibody or antigen-binding fragment comprises the amino acid sequence set forth in SEQ ID NO: 27. In some embodiments, the V_{H} and V_{L} regions of the antibody or antigen-binding fragment thereof comprise the amino acid sequences set forth in SEQ ID NO: 24 and 27, respectively, or any antibody or antigen-binding fragment thereof that has at least 90% sequence identity to any of the above V_{H} and V_{L}, such as at least 90%, 91%, 92%, 93%, 94%, 95%, 96%, 97%, 98%, or 99% sequence identity thereto.

For example, the V_{H} and V_{L} regions of the antibody or antigen-binding fragment thereof provided therein comprise the amino acid sequence set forth in SEQ ID NO: 24 and 27, respectively.

Among the CARs provided herein is a CAR in which the antibody, such as an anti-CD19 antibody, or antibody fragment, in the provided CAR, comprises a V_{H} region amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 24, and a V_{L} region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 28.

In some embodiments, the V_{H} region of the antibody or antigen-binding fragment thereof comprises a CDR-H1, a CDR-H2, a CDR-H3, respectively, comprising the amino acid sequences of CDR-H1, CDR-H2, and CDR-H3 contained within the V_{H} region amino acid sequence set forth in SEQ ID NO: 24; and comprises a CDR-L1, a CDR-L2, a CDR-L3, respectively, comprising the amino acid sequences of CDR-L1, CDR-L2, and CDR-L3, respectively contained within the V_{L} region amino acid sequence set forth in SEQ ID NO: 28.

In some embodiments, the V_{H} region of the antibody or antigen-binding fragment thereof comprise the amino acid sequence set forth in SEQ ID NO: 24 and the V_{L} region of the antibody or antigen-binding fragment comprises the amino acid sequence set forth in SEQ ID NO: 28. In some embodiments, the V_{H} and V_{L} regions of the antibody or antigen-binding fragment thereof comprise the amino acid sequences set forth in SEQ ID NO: 24 and 28, respectively, or any antibody or antigen-binding fragment thereof that has at least 90% sequence identity to any of the above V_{H} and V_{L}, such as at least 90%, 91%, 92%, 93%, 94%, 95%, 96%, 97%, 98%, or 99% sequence identity thereto.

For example, the V_{H} and V_{L} regions of the antibody or antigen-binding fragment thereof provided therein comprise the amino acid sequence set forth in SEQ ID NO: 24 and 28, respectively.

Among the CARs provided herein is a CAR in which the antibody, such as an anti-CD19 antibody, or antibody fragment, in the provided CAR, comprises a V_{H} region amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 24, and a V_{L} region comprising an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 29.

In some embodiments, the V_{H} region of the antibody or antigen-binding fragment thereof comprises a CDR-H1, a CDR-H2, a CDR-H3, respectively, comprising the amino acid sequences of CDR-H1, CDR-H2, and CDR-H3 contained within the V_{H} region amino acid sequence set forth in SEQ ID NO: 24; and comprises a CDR-L1, a CDR-L2, a CDR-L3, respectively, comprising the amino acid sequences of CDR-L1, CDR-L2, and CDR-L3, respectively contained within the V_{L} region amino acid sequence set forth in SEQ ID NO: 29.

In some embodiments, the V_{H} region of the antibody or antigen-binding fragment thereof comprise the amino acid sequence set forth in SEQ ID NO: 24 and the V_{L} region of the antibody or antigen-binding fragment comprises the amino acid sequence set forth in SEQ ID NO: 29. In some embodiments, the V_{H} and V_{L} regions of the antibody or antigen-binding fragment thereof comprise the amino acid sequences set forth in SEQ ID NO: 24 and 29, respectively, or any antibody or antigen-binding fragment thereof that has at least 90% sequence identity to any of the above V_{H} and V_{L}, such as at least 90%, 91%, 92%, 93%, 94%, 95%, 96%, 97%, 98%, or 99% sequence identity thereto.

For example, the V_{H} and V_{L} regions of the antibody or antigen-binding fragment thereof provided therein comprise the amino acid sequence set forth in SEQ ID NO: 24 and 29, respectively.

In some embodiments, the antibody or antigen-binding fragment thereof, in the provided CAR, is a single-chain antibody fragment, such as a single chain variable fragment (scFv) or a diabody or a single domain antibody (sdAb). In some embodiments, the antibody or antigen-binding fragment is a single domain antibody comprising only the V_{H} region. In some embodiments, the antibody or antigen binding fragment is based on an scFv comprising a heavy chain variable (V_{H}) region and a light chain variable (V_{L}) region. In some embodiments, the single-chain antibody fragment includes one or more linkers joining two antibody domains or regions, such as a heavy chain variable (V_{H}) region and a light chain variable (V_{L}) region. The linker typically is a peptide linker, e.g., a flexible and/or soluble peptide linker. Among the linkers are those rich in glycine and serine and/or in some cases threonine. In some embodiments, the linkers further include charged residues such as lysine and/or glutamate, which can improve solubility. In some embodiments, the linkers further include one or more proline.

Accordingly, the provided CARs contain anti-CD19 antibodies that include single-chain antibody fragments, such as scFvs and diabodies, particularly human single-chain antibody fragments, typically comprising linker(s) joining two antibody domains or regions, such V_{H} and V_{L} regions. The linker typically is a peptide linker, e.g., a flexible and/or soluble peptide linker, such as one rich in glycine and serine. In some embodiments, the CD19-binding domain comprises a linker between the V_{H} and V_{L} regions. In some embodiments, in order from N- to C-terminus, the CD19-binding domain comprises one of the V_{H} and V_{L} regions, a linker, and the other of the V_{H} and V_{L} regions. In some embodiments, the linker is set forth in SEQ ID NO: 19. Thus, in some embodiments, in order from N- to C-terminus, the CD19-binding domain comprises one of the V_{H} and V_{L} regions, the linker set forth in SEQ ID NO: 19, and the other of the V_{H} and V_{L} regions.

In some aspects, the linkers rich in glycine and serine (and/or threonine) include at least 80%, 85%, 90%, 91%, 92%, 93%, 94%, 95%, 96%, 97%, 98%, or 99% such amino acid(s). In some embodiments, they include at least at or about 50%, 55%, 60%, 70%, or 75%, glycine, serine, and/or threonine. In some embodiments, the linker is comprised substantially entirely of glycine, serine, and/or threonine. The linkers generally are between about 5 and about 50 amino acids in length, typically between at or about 10 and at or about 30, e.g., 10, 11, 12, 13, 14, 15, 16, 17, 18, 19, 20, 21, 22, 23, 24, 25, 26, 27, 28, 29, or 30, and in some examples between 10 and 25 amino acids in length. Exemplary linkers include linkers having various numbers of repeats of the sequence GGGGS (4GS; SEQ ID NO: 20), such as between 2, 3, 4 and 5 repeats of such a sequence. Exemplary linkers include those having or consisting of an sequence set forth in SEQ ID NO: 18 (GGGGSGGGGSGGGGS). Exemplary linkers further include those having or consisting of the sequence set forth in SEQ ID NO: 19 (GSTSGSGKPGSGEGSTKG), SEQ ID NO: 21 (GGGGSGGGGS), and SEQ ID NO: 22 (GGGGSGGGGSGGGGSGGGGS).

Accordingly, in some embodiments, the provided embodiments include single-chain antibody fragments, e.g., scFvs, comprising one or more of the aforementioned linkers, such as glycine/serine rich linkers, including linkers having repeats of GGGS (SEQ ID NO: 20), such as the linker set forth in SEQ ID NO: 18, 21, or 22.

Accordingly, in some embodiments, the provided embodiments include single-chain antibody fragments, e.g., scFvs, comprising one or more of the aforementioned linkers, such as glycine/serine rich linkers, including linkers having repeats of GGGGS (SEQ ID NO: 18), such as the linker set forth in SEQ ID NO: 20, 21, or 22. In some embodiments, the linker comprises the sequence set forth in SEQ ID NO: 18. In some embodiments, the linker comprises the sequence set forth in SEQ ID NO: 20. In some embodiments, the linker comprises the sequence set forth in SEQ ID NO: 21. In some embodiments, the linker comprises the sequence set forth in SEQ ID NO: 22.

In some embodiments, the V_{H} region may be amino terminal to the V_{L} region. In some embodiments, the V_{H} region may be carboxy terminal to the V_{L} region. In particular embodiments, the fragment may include a V_{H} region or portion thereof, followed by the linker, followed by a V_{L} region or portion thereof. In other embodiments, the fragment may include the V_{L} region or portion thereof, followed by the linker, followed by the V_{H} region or portion thereof.

In some aspects, a CD19 scFv provided herein comprises the amino acid sequence set forth in SEQ ID NO: 67, or has an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 67. In some aspects, an scFv provided herein comprises the amino acid sequence set forth in SEQ ID NO: 67, or has an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 67. In some aspects, an scFv provided herein comprises the amino acid sequence set forth in SEQ ID NO: 67. In some aspects, an scFv provided herein comprises an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 67.

In some aspects, an scFv provided herein comprises the amino acid sequence set forth in SEQ ID NO: 30, or has an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 30. In some aspects, an scFv provided herein comprises the amino acid sequence set forth in SEQ ID NO: 30. In some aspects, an scFv provided herein comprises an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 30.

In some aspects, an scFv provided herein comprises the amino acid sequence set forth in SEQ ID NO: 31, or has an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 31. In some aspects, an scFv provided herein comprises the amino acid sequence set forth in SEQ ID NO: 31. In some aspects, an scFv provided herein comprises an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 31.

In some aspects, an scFv provided herein comprises the amino acid sequence set forth in SEQ ID NO: 32, or has an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 32. In some aspects, an scFv provided herein comprises the amino acid sequence set forth in SEQ ID NO: 32. In some aspects, an scFv provided herein comprises an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 32.

In some aspects, an scFv provided herein comprises the amino acid sequence set forth in SEQ ID NO: 33, or has an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 33. In some aspects, an scFv provided herein comprises the amino acid sequence set forth in SEQ ID NO: 33. In some aspects, an scFv provided herein comprises an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 33.

In some aspects, an scFv provided herein comprises the amino acid sequence set forth in SEQ ID NO: 34, or has an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 34. In some aspects, an scFv provided herein comprises the amino acid sequence set forth in SEQ ID NO: 34. In some aspects, an scFv provided herein comprises an amino acid sequence having at least at or about 90%, at or about 91%, at or about 92%, at or about 93%, at or about 94%, at or about 95%, at or about 96%, at or about 97%, at or about 98%, or at or about 99% sequence identity to the amino acid sequence set forth in SEQ ID NO: 34.

Among the provided CARs is a CAR in which the CD19-binding domain contains a V_{H} region comprising the sequence set forth in SEQ ID NO: 1 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 1; and contains a V_{L} region comprising the sequence set forth in SEQ ID NO: 2 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 2. In some embodiments, the CD19-binding domain of the provided CAR contains a V_{H} region that has a CDRH1, a CDRH2 and a CDRH3 comprising the amino acid sequence of SEQ ID NOs: 3, 4, and 5, respectively, or 3, 4, and 6 respectively, and a V_{L} region that has a CDRL1, a CDRL2 and a CDRL3 comprising the amino acid sequence of SEQ ID NOs: 7, 8, and 10, respectively; 7, 9, and 10, respectively; 7, 8, and 11, respectively; or 7, 9, and 11, respectively. In some embodiments, the V_{H} region comprises the sequence set forth in SEQ ID NO: 1 and the V_{L} region comprises the sequence set forth in SEQ ID NO: 2.

Among the provided CARs is a CAR in which the CD19-binding domain contains a V_{H} region comprising the sequence set forth in SEQ ID NO: 23 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 23; and contains a V_{L} region comprising the sequence set forth in SEQ ID NO: 25 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 25. In some embodiments, the CD19-binding domain of the provided CAR contains a V_{H} region that has a CDRH1, a CDRH2 and a CDRH3 comprising the amino acid sequence of SEQ ID NOs: 35, 36, and 37, respectively, and a V_{L} region that has a CDRL1, a CDRL2 and a CDRL3 comprising the amino acid sequence of SEQ ID NOs: 38, 39, and 40. In some embodiments, the V_{H} region comprises the sequence set forth in SEQ ID NO: 23 and the V_{L} region comprises the sequence set forth in SEQ ID NO: 25.

Among the provided CARs is a CAR in which the CD19-binding domain contains a V_{H} region comprising the sequence set forth in SEQ ID NO: 23 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 23; and contains a V_{L} region comprising the sequence set forth in SEQ ID NO: 26 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 26. In some embodiments, the CD19-binding domain of the provided CAR contains a V_{H} region that has a CDRH1, a CDRH2 and a CDRH3 comprising the amino acid sequence of SEQ ID NOs: 35, 36, and 37, respectively, and a V_{L} region that has a CDRL1, a CDRL2 and a CDRL3 comprising the amino acid sequence of SEQ ID NOs: 41, 42, and 43, respectively. In some embodiments, the V_{H} region comprises the sequence set forth in SEQ ID NO: 23 and the V_{L} region comprises the sequence set forth in SEQ ID NO: 26.

Among the provided CARs is a CAR in which the CD19-binding domain contains a V_{H} region comprising the sequence set forth in SEQ ID NO: 24 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 24; and contains a V_{L} region comprising the sequence set forth in SEQ ID NO: 27 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 27. In some embodiments, the CD19-binding domain of the provided CAR contains a V_{H} region that has a CDRH1, a CDRH2 and a CDRH3 comprising the amino acid sequence of SEQ ID NOs: 35, 36, and 37, respectively, and a V_{L} region that has a CDRL1, a CDRL2 and a CDRL3 comprising the amino acid sequence of SEQ ID NOs: 44, 45, and 46, respectively. In some embodiments, the V_{H} region comprises the sequence set forth in SEQ ID NO: 24 and the V_{L} region comprises the sequence set forth in SEQ ID NO: 27.

Among the provided CARs is a CAR in which the CD19-binding domain contains a V_{H} region comprising the sequence set forth in SEQ ID NO: 24 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 24; and contains a V_{L} region comprising the sequence set forth in SEQ ID NO: 28 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 28. In some embodiments, the CD19-binding domain of the provided CAR contains a V_{H} region that has a CDRH1, a CDRH2 and a CDRH3 comprising the amino acid sequence of SEQ ID NOs: 35, 36, and 37, respectively, and a V_{L} region that has a CDRL1, a CDRL2 and a CDRL3 comprising the amino acid sequence of SEQ ID NOs: 44, 45, and 47. In some embodiments, the V_{H} region comprises the sequence set forth in SEQ ID NO: 24 and the V_{L} region comprises the sequence set forth in SEQ ID NO: 28.

Among the provided CARs is a CAR in which the CD19-binding domain contains a V_{H} region comprising the sequence set forth in SEQ ID NO: 24 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 24; and contains a V_{L} region comprising the sequence set forth in SEQ ID NO: 29 or an amino acid sequence having at least at or about 90%, at or about 91%, at or about 92%, at or about 93%, at or about 94%, at or about 95%, at or about 96%, at or about 97%, at or about 98%, or at or about 99% identity to SEQ ID NO: 29. In some embodiments, the CD19-binding domain of the provided CAR contains a V_{H} region that has a CDRH1, a CDRH2 and a CDRH3 comprising the amino acid sequence of SEQ ID NOs: 35, 36, and 37, respectively, and a V_{L} region that has a CDRL1, a CDRL2 and a CDRL3 comprising the amino acid sequence of SEQ ID NOs: 48, 49, and 50, respectively. In some embodiments, the V_{H} region comprises the sequence set forth in SEQ ID NO: 24 and the V_{L} region comprises the sequence set forth in SEQ ID NO: 29.

Among the antibodies, e.g., antigen-binding fragments, in the provided CARs, are human antibodies. In some embodiments of a provided human anti-CD19 antibody, e.g., antigen-binding fragments, the human antibody contains a V_{H} region that comprises a portion having at least 95%, 96%, 97%, 98%, 99%, or 100% sequence identity to an amino acid sequence encoded by a germline nucleotide human heavy chain V segment, a portion having at least 95%, 96%, 97%, 98%, 99%, or 100% sequence identity to an amino acid sequence encoded by a germline nucleotide human heavy chain D segment, and/or a portion having at least 95%, 96%, 97%, 98%, 99%, or 100% sequence identity to an amino acid sequence encoded by a germline nucleotide human heavy chain J segment; and/or contains a V_{L} region that comprises a portion having at least 95%, 96%, 97%, 98%, 99%, or 100% sequence identity to an amino acid sequence encoded by a germline nucleotide human kappa or lambda chain V segment, and/or a portion having at least 95%, 96%, 97%, 98%, 99%, or 100% sequence identity to an amino acid sequence encoded by a germline nucleotide human kappa or lambda chain J segment. In some embodiments, the portion of the V_{H} region corresponds to the CDR-H1, CDR-H2 and/or CDR-H3. In some embodiments, the portion of the V_{H} region corresponds to the framework region 1 (FR1), FR2, FR2 and/or FR4. In some embodiments, the portion of the V_{L} region corresponds to the CDR-L1, CDR-L2 and/or CDR-L3. In some embodiments, the portion of the V_{L} region corresponds to the FR1, FR2, FR2 and/or FR4.

In some embodiments, the human antibody, e.g., antigen-binding fragment, contains a CDR-H1 having at least 95%, 96%, 97%, 98%, 99%, or 100% sequence identity to an amino acid sequence of the corresponding CDR-H1 region within a sequence encoded by a germline nucleotide human heavy chain V segment. For example, the human antibody in some embodiments contains a CDR-H1 having a sequence that is 100% identical or with no more than one, two or three amino acid differences as compared to the corresponding CDR-H1 region within a sequence encoded by a germline nucleotide human heavy chain V segment.

In some embodiments, the human antibody, e.g., antigen-binding fragment, contains a CDR-H2 having at least 95%, 96%, 97%, 98%, 99%, or 100% sequence identity to an amino acid sequence of the corresponding CDR-H2 region within a sequence encoded by a germline nucleotide human heavy chain V segment. For example, the human antibody in some embodiments contains a CDR-H2 having a sequence that is 100% identical or with no more than one, two or three amino acid difference as compared to the corresponding CDR-H2 region within a sequence encoded by a germline nucleotide human heavy chain V segment.

In some embodiments, the human antibody, e.g., antigen-binding fragment, contains a CDR-H3 having at least 95%, 96%, 97%, 98%, 99%, or 100% sequence identity to an amino acid sequence of the corresponding CDR-H3 region within a sequence encoded by a germline nucleotide human heavy chain V segment, D segment and J segment. For example, the human antibody in some embodiments contains a CDR-H3 having a sequence that is 100% identical or with no more than one, two or three amino acid differences as compared to the corresponding CDR-H3 region within a sequence encoded by a germline nucleotide human heavy chain V segment, D segment and J segment.

In some embodiments, the human antibody, e.g., antigen-binding fragment, contains a CDR-L1 having at least 95%, 96%, 97%, 98%, 99%, or 100% sequence identity to an amino acid sequence of the corresponding CDR-L1 region within a sequence encoded by a germline nucleotide human light chain V segment. For example, the human antibody in some embodiments contains a CDR-L1 having a sequence that is 100% identical or with no more than one, two or three amino acid differences as compared to the corresponding CDR-L1 region within a sequence encoded by a germline nucleotide human light chain V segment.

In some embodiments, the human antibody, e.g., antigen-binding fragment, contains a CDR-L2 having at least 95%, 96%, 97%, 98%, 99%, or 100% sequence identity to an amino acid sequence of the corresponding CDR-L2 region within a sequence encoded by a germline nucleotide human light chain V segment. For example, the human antibody in some embodiments contains a CDR-L2 having a sequence that is 100% identical or with no more than one, two or three amino acid difference as compared to the corresponding CDR-L2 region within a sequence encoded by a germline nucleotide human light chain V segment.

In some embodiments, the human antibody, e.g., antigen-binding fragment, contains a CDR-L3 having at least 95%, 96%, 97%, 98%, 99%, or 100% sequence identity to an amino acid sequence of the corresponding CDR-L3 region within a sequence encoded by a germline nucleotide human light chain V segment and J segment. For example, the human antibody in some embodiments contains a CDR-L3 having a sequence that is 100% identical or with no more than one, two or three amino acid differences as compared to the corresponding CDR-L3 region within a sequence encoded by a germline nucleotide human light chain V segment and J segment.

In some embodiments, the human antibody, e.g., antigen-binding fragment, contains a framework region that contains human germline gene segment sequences. For example, in some embodiments, the human antibody contains a V_{H} region in which the framework region, e.g. FR1, FR2, FR3 and FR4, has at least 95%, 96%, 97%, 98%, 99%, or 100% sequence identity to a framework region encoded by a human germline antibody segment, such as a V segment and/or J segment. In some embodiments, the human antibody contains a V_{L} region in which the framework region e.g. FR1, FR2, FR3 and FR4, has at least 95%, 96%, 97%, 98%, 99%, or 100% sequence identity to a framework region encoded by a human germline antibody segment, such as a V segment and/or J segment. For example, in some such embodiments, the framework region sequence contained within the V_{H} region and/or V_{L} region differs by no more than 10 amino acids, such as no more than 9, 8, 7, 6, 5, 4, 3, 2 or 1 amino acid, compared to the framework region sequence encoded by a human germline antibody segment.

### B. Spacer

In some embodiments, the CAR further include a spacer domain (in some cases also called a spacer region) that is located between the extracellular binding domain and the transmembrane domain. In some embodiments, spacer contains a hinge region sequence, which in some aspects is a sequence that promotes receptor dimerization. In some embodiments, the spacer is or includes at least a portion of an immunoglobulin constant region or variant or modified version thereof. In some embodiments, the portion of the immunoglobulin constant region includes a hinge region, e.g., an IgG4 hinge region, and/or a C_{H}1, C_{H}2 or C_{H}3 and/or Fc region. In some embodiments, the constant region or portion is of a human IgG, such as IgG4 or IgG1. In some aspects, the portion of the constant region serves as a spacer region between the extracellular binding domain and transmembrane domain. In some embodiments, the constant region or portion is of IgD.

In some embodiments, the spacer is or comprises a glycine-serine rich sequence or other flexible linker such as known flexible linkers.

In some embodiments, the spacer is or includes at least a portion of human CD4, CD8, or CD28 proteins. In some embodiments, the spacer is or includes a hinge region from CD4, CD8, or CD28 extracellular domains. In some embodiments, the spacer is or includes a CD8 hinge domain sequence set forth in SEQ ID NO: 127. In some embodiments, the spacer is or includes a CD28 hinge domain spacer sequence set forth in SEQ ID NO: 128. In some embodiments, the spacer is or includes a CD28 hinge domain spacer sequence set forth in SEQ ID NO: 129.

In some embodiments, the length of the spacer is adjusted to optimize the biophysical synapse distance between the CAR-expressing cell and the target of the CAR, such as a CAR-expressing T-cell, and the target of the CAR, such as a CD19-expressing cell. In some embodiments, the CAR is expressed by a T cell, and the length of the spacer is adjusted to a length that is compatible for T cell activation or to optimize CAR T-cell performance.

In some embodiments, the spacer can be of a length that provides for increased responsiveness of the cell following antigen binding, as compared to in the absence of the spacer or as compared to an alternative spacer of a different length (e.g. longer in length). In some examples, the spacer is at or about 12 to 15 amino acids in length. In some examples, the spacer is at or about 220 to 240 amino acids in length.

Exemplary spacers include those having at least at or about 10 to at or about 300 amino acids, at or about 10 to at or about 229 amino acids, at or about 10 to at or about 200 amino acids, at or about 10 to at or about 175 amino acids, at or about 10 to at or about 150 amino acids, at or about 10 to at or about 125 amino acids, at or about 10 to at or about 100 amino acids, at or about 10 to at or about 75 amino acids, at or about 10 to at or about 50 amino acids, at or about 10 to at or about 40 amino acids, at or about 10 to at or about 30 amino acids, at or about 10 to at or about 20 amino acids, or at or about 12 to at or about 15 amino acids in length, and including any integer between the endpoints of any of the listed ranges. Exemplary spacers include those having at least at or about at or about 50 to at or about 175 amino acids, at or about 50 to at or about 150 amino acids, at or about 10 to at or about 125 amino acids, at or about 50 to at or about 100 amino acids, at or about 100 to at or about 300 amino acids, at or about 100 to at or about 250 amino acids, at or about 125 to at or about 250 amino acids, or at or about 200 to at or about 250 amino acids, and including any integer between the endpoints of any of the listed ranges. In some embodiments, a spacer is at least at or about 12 amino acids, at least at or about 119 amino acids, at least at or about 125 amino acids, at least at or about 200 amino acids, or at least at or about 220 amino acids, or at least at or about 225 amino acids in length. In some embodiments, a spacer is at least at or about 13 amino acids, at least at or about 120 amino acids, at least at or about 125 amino acids, at least at or about 200 amino acids, or at least at or about 220 amino acids, or at least at or about 229 amino acids in length. In some embodiments, a spacer is at or about 11, 12, 13, 14, 15, 16, 17, 18, 19, 20, 21, 22, 23, 24, 25 amino acids or less in length. In some embodiments, the spacer is at least at or about 100 amino acids in length, such as at least at or about 110, 111, 112, 113, 114, 115, 116, 117, 118, 119, 120, 121, 122, 123, 124, 125, 130, 135, 140, 145, 150, 160, 170, 180, 190, 200, 210, 220, 230, 240, or 250 amino acids in length. In some embodiments, the spacer is between 220 and 240 amino acids in length.

In some embodiments, the spacer is at least at or about 125 to at or about 300 amino acids, at or about 125 to at or about 250 amino acids, at or about 125 to at or about 230 amino acids, at or about 125 to at or about 200 amino acids, at or about 125 to at or about 180 amino acids, at or about 125 to at or about 150 amino acids, at or about 150 to at or about 300 amino acids, at or about 150 to at or about 250 amino acids, at or about 150 to at or about 230 amino acids, at or about 150 to at or about 200 amino acids, at or about 150 to at or about 180 amino acids, at or about 180 to at or about 300 amino acids, at or about 180 to at or about 250 amino acids, at or about 180 to at or about 230 amino acids, at or about 180 to at or about 200 amino acids, at or about 200 to at or about 300 amino acids, at or about 200 to at or about 250 amino acids, at or about 200 to at or about 230 amino acids, at or about 230 to at or about 300 amino acids, at or about 230 to at or about 250 amino acids in length or 250 to at or about 300 amino acids in length.

Exemplary spacers include an IgG hinge alone, an IgG hinge linked to one or more of a C_{H}2 and C_{H}3 domain, IgG hinge linked to the C_{H}3 domain. In some embodiments, the spacer includes an IgG hinge alone. In some embodiments, the IgG hinge, C_{H}2 and/or C_{H}3 can be derived all or in part from IgG4 or IgG2, such as all or in part from human IgG4 or human IgG2. In some embodiments, the spacer can be a chimeric polypeptide containing one or more of a hinge, C_{H}2 and/or C_{H}3 sequence(s) derived from IgG4, IgG2, and/or IgG2 and IgG4. In some embodiments, the hinge region comprises all or a portion of an IgG4 hinge region. In some embodiments, the hinge region comprises all or a portion of an IgG4 hinge region and/or of an IgG2 hinge region, wherein the IgG4 hinge region is optionally a human IgG4 hinge region and the IgG2 hinge region is optionally a human IgG2 hinge region; the C_{H}2 region comprises all or a portion of an IgG4 C_{H}2 region and/or of an IgG2 C_{H}2 region, wherein the IgG4 C_{H}2 region is optionally a human IgG4 C_{H}2 region and the IgG2 C_{H}2 region is optionally a human IgG2 C_{H}2 region; and/or the C_{H}3 region comprises all or a portion of an IgG4 C_{H}3 region and/or of an IgG2 C_{H}3 region, wherein the IgG4 C_{H}3 region is optionally a human IgG4 C_{H}3 region and the IgG2 C_{H}3 region is optionally a human IgG2 C_{H}3 region. In some embodiments, the hinge, C_{H}2 and C_{H}3 comprises all or a portion of each of a hinge region, C_{H}2 and C_{H}3 from IgG4. In some embodiments, the hinge region is chimeric and comprises a hinge region from human IgG4 and human IgG2; the C2 region is chimeric and comprises a C_{H}2 region from human IgG4 and human IgG2; and/or the C_{H}3 region is chimeric and comprises a C3 region from human IgG4 and human IgG2. In some embodiments, the spacer comprises an IgG4/2 chimeric hinge or a modified IgG4 hinge comprising at least one amino acid replacement compared to human IgG4 hinge region; an human IgG2/4 chimeric C2 region; and a human IgG4 C_{H}3 region.

In some examples, the spacer is at or about 12 amino acids in length or is no more than at or about 12 amino acids in length. In some examples, the spacer is at or about 15 amino acids in length or is no more than at or about 15 amino acids in length.

In some embodiments, the spacer comprises or consists of all or a portion of an immunoglobulin hinge or a modified version thereof. In some embodiments, the spacer is at or about 15 amino acids or less in length. In some embodiments, the spacer comprises or consists of all or a portion of an immunoglobulin hinge, optionally an IgG4 hinge, or a modified version thereof and/or comprises about 15 amino acids or less. In some embodiments, the spacer is at or about 13 amino acids in length and/or comprises or consists of all or a portion of an immunoglobulin hinge, optionally an IgG4, or a modified version thereof. In some embodiments, the spacer is at or about 12 amino acids in length and/or comprises or consists of all or a portion of an immunoglobulin hinge, optionally an IgG4, or a modified version thereof. In some embodiments, the spacer comprises the formula X₁PPX₂P (SEQ ID NO: 51), where X₁ is glycine, cysteine or arginine and X₂ is cysteine or threonine.

In some embodiments, the spacer contains only a hinge region of an IgG, such as only a hinge of IgG4 or IgG1, such as the hinge only spacer set forth in SEQ ID NO: 12 or 113, such as encoded by the sequence set forth in SEQ ID NO: 114. In some embodiments, the spacer is an Ig hinge, e.g., and IgG4 hinge, linked to a C_{H}2 and/or C_{H}3 domains. In some embodiments, the spacer is an Ig hinge, e.g., an IgG4 hinge, linked to C_{H}2 and C_{H}3 domains, such as set forth in SEQ ID NO: 13. In some embodiments, the spacer the spacer is an Ig hinge, e.g., an IgG4 hinge, linked to a C_{H}3 domain only, such as set forth in SEQ ID NO: 115. In some embodiments, the spacer is or comprises a glycine-serine rich sequence or other flexible linker such as known flexible linkers. In some embodiments, the constant region or portion is of IgD. In some embodiments, the spacer has the sequence set forth in SEQ ID NO: 116. In some embodiments, the spacer has a sequence of amino acids that exhibits at least or at least about 85%, 86%, 87%, 88%, 89%, 90%, 91%, 92%, 93%, 94%, 95%, 96%, 97%, 98%, 99% or more sequence identity to any of SEQ ID NOs: 12, 13, 115 and 116.

In some embodiments, the spacer is a hinge region sequence set forth in any one of SEQ ID NOs: 117, 118, 119, 120, 121, 122, 123, and 124.

In certain cases, the spacer has a methionine residue at the C-terminus. In some embodiments, the spacer comprises or consists of the sequence of SEQ ID NO: 12, or a variant of any of the foregoing having at least 85%, 86%, 87%, 88%, 89%, 90%, 91%, 92%, 93%, 94%, 95%, 96%, 97%, 98%, 99% or more sequence identity thereto.

In some embodiments, the spacer and can contain one or more single amino acid mutations in one or more domains of the immunoglobulin, such as in one or more domains of the hinge, CH2 or CH3 region. In some embodiments, the spacer can be from all or in part from IgG4 and/or IgG2 and can contain mutations, such as one or more single amino acid mutations in one or more domains. In some examples, the amino acid modification is a substitution of a proline (P) for a serine (S) in the hinge region of an IgG4. In some embodiments, the spacer is or contains a IgG4 hinge that is a variant IgG4 hinge region comprising substitution of amino acids CPSC to CPPC compared to the wild-type IgG4 hinge region. In some embodiments, the amino acid modification is a substitution of a glutamine (Q) for an asparagine (N) to reduce glycosylation heterogeneity, such as an N177Q mutation at position 177, in the C_{H}2 region, of the full-length IgG4 Fe sequence set forth in SEQ ID NO: 53 or an N176Q at position 176, in the C_{H}2 region, of the full-length IgG2 Fe sequence set forth in SEQ ID NO: 52.

In some embodiments, the spacer containing a hinge and constant region sequences of an immunoglobulin. In some embodiments, the spacer is or comprises an IgG4/2 chimeric hinge or a modified IgG4 hinge; an IgG2/4 chimeric C_{H}2 region; and an IgG4 C_{H}3 region. In some embodiments, the spacer is about 228 or 229 amino acids in length. In some embodiments, the spacer is set forth in SEQ ID NO: 13. In some embodiments, the spacer is or contains an amino acid sequence having at least at or about 85%, 86%, 87%, 88%, 89%, 90%, 91%, 92%, 93%, 94%, 95%, 96%, 97%, 98% or 99% identity to SEQ ID NO: 13. In some embodiments, the spacer is or contains the sequence set forth in SEQ ID NO: 13.

Exemplary spacers include, but are not limited to, those described in Hudecek et al. (2013) Clin. Cancer Res., 19:3153, Hudecek et al. (2015) Cancer Immunol. Res., 3(2):125-135, or WO2014031687.

In some embodiments, the nucleotide sequence of the spacer is optimized to reduce RNA heterogeneity upon expression. In some embodiments, the nucleotide sequence of the spacer is optimized to reduce cryptic splice sites or reduce the likelihood of a splice event at a splice site.

In some embodiments, the spacer has an amino acid sequence that exhibits at least 85%, 86%, 87%, 88%, 89%, 90%, 91%, 92%, 93%, 94%, 95%, 96%, 97%, 98%, 99% or more sequence identity to SEQ ID NO: 12 or 13, encoded by a polynucleotide that has been optionally optimized for codon usage and/or to reduce RNA heterogeneity. Methods to reduce RNA heterogeneity, such as by removing cryptic splice donor and/or acceptor sites, are described below. Observations have shown that cryptic splice donor and/or acceptor sites are present in the spacer region of certain immunoglobulin spacers when present in a CAR. In some embodiments, the spacer in a provided CAR is encoded by a polynucleotide in which one or more cryptic splice donor and/or acceptor sites are eliminated and/or are modified to reduce heterogeneity of the RNA transcribed from the construct, such as mRNA, following expression in a cell.

### C. Transmembrane Domain

The CAR includes a transmembrane domain (also referred to as transmembrane region) linking the extracellular domain containing the antigen binding domain (e.g., the CD19-binding domain) and the intracellular domain. In some embodiments, the V_{H} or the V_{L} of the binding domain most proximal to the cellular membrane is linked to the transmembrane domain. In some embodiments, the transmembrane domain is fused to the extracellular domain.

In some embodiments, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane domains include those derived from (i.e. comprise at least the transmembrane domain(s) of) the alpha, beta or zeta chain of the T-cell receptor, CD3 epsilon, CD4, CD5, CD8, CD9, CD16, CD22, CD28, CD33, CD37, CD45, CD64, CD80, CD86, CD134, CD137, and/or CD154. In some embodiments, the transmembrane domain can be a CD4 transmembrane. In some embodiments, the transmembrane domain is a transmembrane domain of human CD4 or variant thereof. In some embodiments, the transmembrane domain is a CD8 transmembrane domain. In some embodiments, the transmembrane domain is a transmembrane domain of human CD8 or variant thereof. In some embodiments, the transmembrane domain is a CD28 transmembrane domain. In some embodiments, the transmembrane domain is a transmembrane domain of human CD28 or variant thereof.

In some embodiments, the transmembrane domain is a CD28 transmembrane domain that comprises the sequence of amino acids set forth in SEQ ID NO: 15. In some embodiments, the transmembrane domain of the receptor is a transmembrane domain of human CD28 or variant thereof, e.g., a 2 transmembrane domain of a human CD28 (Accession No.: P10747.1). In some embodiments, the transmembrane domain is a transmembrane domain that comprises the sequence of amino acids set forth in SEQ ID NO: 15 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 15. In some embodiments, the transmembrane domain is a transmembrane domain that comprises the sequence of amino acids set forth in SEQ ID NO: 125 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 125.

In some embodiments, the transmembrane domain is or contains SEQ ID NO: 15 or an amino acid sequence having at least at or about 90%, 91%, 92%, 93%, 94%, 95%, 96%, 97%, 98% or 99% sequence identity to SEQ ID NO: 15. In some embodiments, the transmembrane domain is or contains the sequence set forth in SEQ ID NO: 15.

In some embodiments, the transmembrane domain of the is a transmembrane domain of a human CD8a. In some embodiments, the transmembrane domain is a transmembrane domain that comprises the sequence of amino acids set forth in SEQ ID NO: 126 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 126.

Alternatively, the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s).

### D. Intracellular Signaling Domain

The receptor, e.g., the CAR, generally includes an intracellular signaling region comprising at least one intracellular signaling component or components. T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such classes of cytoplasmic signaling sequences. Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the intracellular signaling domain of the CAR.

In some embodiments, upon ligation of the CAR, the cytoplasmic domain or intracellular signaling region of the CAR stimulates and/or activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptor to initiate signal transduction following antigen receptor engagement, and/or any derivative or variant of such molecules, and/or any synthetic sequence that has the same functional capability.

In some embodiments, the receptor includes an intracellular component or signaling domain of a TCR complex, such as a TCR CD3 chain that mediates T-cell activation and cytotoxicity, e.g., CD3 zeta (CD3-ζ) chain. Thus, in some aspects, the CD19 binding domain is linked to one or more cell signaling modules. In some embodiments, cell signaling modules include CD3 intracellular signaling domains and/or other CD transmembrane domains. In some embodiments, the receptor, e.g., CAR, further includes a portion of one or more additional molecules such as Fc receptor y, CD8, CD4, CD25, or CD16.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

In some aspects, the CAR includes a primary cytoplasmic signaling sequence that regulates primary stimulation and/or activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR or CD3 zeta, FcR gamma, CD3 gamma, CD3 delta and CD3 epsilon. In some embodiments, the intracellular signaling region in the CAR contain(s) a cytoplasmic signaling domain, portion thereof, or sequence derived from CD3 zeta. In some embodiments the CD3 zeta comprises the sequence of amino acids set forth in SEQ ID NO: 17.

In some embodiments, the intracellular signaling domain comprises a human CD3 zeta stimulatory signaling domain or functional variant thereof, such as an 112 AA cytoplasmic domain of isoform 3 of human CD3((Accession No.: P20963.2) or a CD3 zeta signaling domain as described in U.S. Pat. No. 7,446,190 or U.S. Pat. No. 8,911,993. In some embodiments, the intracellular signaling domain comprises the sequence of amino acids set forth in SEQ ID NO: 17 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 17. In some embodiments, the CD3 zeta is or contains the sequence set forth in SEQ ID NO: 17.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

In some aspects, the same CAR includes both the primary (or activating) cytoplasmic signaling regions and costimulatory signaling components.

In some embodiments, the intracellular signaling regions, e.g., comprising intracellular domain or domains, include the cytoplasmic sequences of a region or domain that is involved in providing costimulatory signal. In some embodiments, the CAR includes a signaling domain (e.g., an intracellular or cytoplasmic signaling domain) and/or transmembrane portion of a costimulatory molecule, such as a T cell costimulatory molecule. Exemplary costimulatory molecules include CD28, 4-1BB, OX40, DAP10, CD2, CD40, CD7, CD27, GITR, and ICOS. In some embodiments, the CAR costimulatory domain is derived from immune-stimulatory receptors such as TACI, BAFF-R, or BCMA.

In some embodiments, the costimulatory molecule from 4-1BB comprises the amino acid sequence set forth in SEQ ID NO: 16. In some embodiments, the intracellular domain comprises an intracellular costimulatory signaling domain of 4-1BB or functional variant or portion thereof, such as a 42-amino acid cytoplasmic domain of a human 4-1BB (Accession No. Q07011.1) or functional variant or portion thereof, such as the sequence of amino acids set forth in SEQ ID NO: 16 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 16.

In some embodiments, the intracellular signaling region or domain comprises an intracellular costimulatory signaling domain of human CD28 or functional variant or portion thereof, such as a 41 amino acid domain thereof and/or such a domain with an LL to GG substitution at positions 186-187 of a native CD28 protein. In some embodiments, the intracellular signaling domain can comprise the sequence of amino acids set forth in SEQ ID NO: 130 or 131 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 130 or 131.

In some embodiments, the intracellular region comprises an intracellular costimulatory signaling domain of 4-1BB or functional variant or portion thereof, such as a 42-amino acid cytoplasmic domain of a human 4-1BB (Accession No. Q07011.1) or functional variant or portion thereof, such as the sequence of amino acids set forth in SEQ ID NO: 16 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 16.

In some embodiments, the costimulatory molecule from 4-1BB is encoded by a polynucleotide that has been optionally optimized for codon usage and/or to reduce RNA heterogeneity, e.g., by removing cryptic splice sites.

In certain embodiments, the intracellular signaling region comprises a CD28 transmembrane and signaling domain linked to a CD3 (e.g., CD3-zeta) intracellular domain. In some embodiments, the intracellular signaling domain comprises a chimeric CD28 and 4-1BB (CD137; TNFRSF9) co-stimulatory domains, linked to a CD3 zeta intracellular domain.

In some embodiments, the CAR encompasses one or more, e.g., two or more, costimulatory domains and a stimulatory or an activation domain, e.g., primary activation domain, in the cytoplasmic portion. Exemplary CARs include intracellular components of CD3-zeta and 4-1BB.

In some embodiments, the CAR contains an extracellular binding domain containing antibody variable chain sequences (e.g., variable heavy chain and variable light chain sequences), a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. For example, in some embodiments, the CAR includes an extracellular binding domain containing antibody variable chain sequences (e.g., variable heavy chain and variable light chain sequences), a spacer (e.g., containing a hinge region, such as an Ig-hinge containing spacer), a transmembrane domain containing all or a portion of a CD28-derived transmembrane domain, a CD28-derived intracellular signaling domain, and a CD3 zeta signaling domain.

In some embodiments, the CAR contains an extracellular binding domain containing antibody variable chain sequences (e.g., variable heavy chain and variable light chain sequences), a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. For example, in some embodiments, the CAR includes an extracellular binding domain containing antibody variable chain sequences (e.g., variable heavy chain and variable light chain sequences), a spacer (e.g., containing a hinge region, such as an Ig-hinge containing spacer), a CD28-derived transmembrane domain, a 4-1BB-derived intracellular signaling domain, and a CD3 zeta-derived signaling domain.

### E. Exemplary CARs

In some embodiments, the CAR comprises, in order from N- to C-terminus: the V_{L} region of the CD19-binding domain set forth in SEQ ID NO: 2, the linker set forth in SEQ ID NO: 19, and the V_{H} region of the CD19-binding domain set forth in SEQ ID NO: 1.

In some embodiments, the CAR comprises, in order from N- to C-terminus: the V_{H} region of the CD19-binding domain set forth in SEQ ID NO: 1, the linker set forth in SEQ ID NO: 19, and the V_{L} region of the CD19-binding domain set forth in SEQ ID NO: 2.

In some embodiments, the CAR comprises, in order from N- to C-terminus: a signal peptide comprising the sequence set forth in SEQ ID NO: 54, a V_{L} region of a CD19-binding domain set forth in SEQ ID NO: 2, a linker comprising the sequence set forth in SEQ ID NO: 19, a V_{H} region of a CD19-binding domain set forth in SEQ ID NO: 1, a hinge comprising the sequence set forth in SEQ ID NO: 12, a transmembrane domain comprising the sequence set forth in SEQ ID NO: 15, a costimulatory domain comprising the sequence set forth in SEQ ID NO: 16, and a signaling domain comprising the sequence set forth in SEQ ID NO: 17.

In some of any embodiments, the CAR comprises the amino acid sequence set forth in SEQ ID NO: 78 or 138, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 78 or 138.

### F. Variants

In certain embodiments, CAR comprising the antibodies include one or more amino acid variations, e.g., substitutions, deletions, insertions, and/or mutations, compared to the sequence of an antibody described herein. Exemplary variants include those designed to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

In certain embodiments, the antibodies include one or more amino acid substitutions, e.g., as compared to an antibody sequence described herein and/or compared to a sequence of a natural repertoire, e.g., human repertoire. Sites of interest for substitutional mutagenesis include the CDRs and FRs. Amino acid substitutions may be introduced into an antibody of interest and the products screened for a desired activity, e.g., retained/improved antigen binding, decreased immunogenicity, improved half-life, and/or improved effector function, such as the ability to promote antibody-dependent cellular cytotoxicity (ADCC) or complement-dependent cytotoxicity (CDC).

In some embodiments, one or more residues within a CDR of a parent antibody (e.g. a humanized or human antibody) is/are substituted. In some embodiments, the substitution is made to revert a sequence or position in the sequence to a germline sequence, such as an antibody sequence found in the germline (e.g., human germline), for example, to reduce the likelihood of immunogenicity, e.g., upon administration to a human subject.

In some embodiments, alterations are made in CDR “hotspots,” residues encoded by codons that undergo mutation at high frequency during the somatic maturation process (see, e.g., Chowdhury, Methods Mol. Biol. 207:179-196 (2008)), and/or residues that contact antigen, with the resulting variant V_{H} or V_{L} being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001)). In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. Such alterations may, for example, be outside of antigen contacting residues in the CDRs. In certain embodiments of the variant V_{H} and V_{L} sequences provided above, each CDR either is unaltered, or contains no more than one, two or three amino acid substitutions.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme or a polypeptide which increases the serum half-life of the antibody.

### G. Polynucleotides Encoding the Chimeric Antigen Receptor

Also provided are polynucleotides encoding the chimeric antigen receptor and/or portions, e.g., chains, thereof. Among the provided polynucleotides are those encoding the bispecific chimeric antigen receptors (e.g., antigen-binding fragment) binding CD19 described herein. The polynucleotides may include those encompassing natural and/or non-naturally occurring nucleotides and bases, e.g., including those with backbone modifications. The terms “nucleic acid molecule”, “nucleic acid”, “sequence of nucleotides”, and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides that comprise the nucleic acid molecule or polynucleotide.

In some cases, the polynucleotide encoding the CD19-binding domain contains a signal sequence that encodes a signal peptide, in some cases encoded upstream of the nucleic acid sequences encoding the CD19-binding domain

In some aspects, the signal sequence may encode a signal peptide derived from a native polypeptide. In other aspects, the signal sequence may encode a heterologous or non-native signal peptide. In some aspects, a non-limiting exemplary signal peptide includes a signal peptide of a GM-CSF signal peptide set forth in SEQ ID NO: 54. In some aspects, a non-limiting exemplary signal peptide includes a signal peptide of a CD33 signal peptide set forth in SEQ ID NO: 55. In some cases, the polynucleotide encoding the CD19 binding domain can contain nucleic acid sequence encoding additional molecules, such as a surrogate marker or other markers, or can contain additional components, such as promoters, regulatory elements and/or multicistronic elements. In some embodiments, the nucleic acid sequence encoding the bispecific tandem CARs can be operably linked to any of the additional components.

In some embodiments, a polynucleotide encoding a CAR comprises or consists of the nucleic acid sequence set forth in SEQ ID NO: 136.

In some embodiments, among CARs provided herein are those encoded by polynucleotides that are optimized, or contain certain features designed for optimization, such as for codon usage, to reduce RNA heterogeneity and/or to modify, e.g., increase or render more consistent among cell product lots, expression, such as surface expression, of the encoded receptor. In some embodiments, polynucleotides, encoding CD19-binding domains, are modified as compared to a reference polynucleotide, such as to remove cryptic or hidden splice sites, to reduce RNA heterogeneity. In some embodiments, polynucleotides, encoding CD19-binding domains, are codon optimized, such as for expression in a mammalian, e.g., human, cell, such as in a human T cell. In some aspects, the modified polynucleotides result in in improved, e.g., increased or more uniform or more consistent level of, expression, e.g., surface expression, when expressed in a cell. Such polynucleotides can be utilized in constructs for generation of engineered cells that express the encoded CD19-binding domains. Thus, also provided are cells expressing the recombinant receptors encoded by the polynucleotides provided herein and uses thereof in adoptive cell therapy, such as treatment of diseases and disorders associated with CD19 expression.

Also provided are cells, such as T cells, engineered to express a polynucleotide encoding a provided polynucleotide, including polynucleotides encoding a CD19-binding domain, and compositions containing such cells. In some embodiments, the polynucleotide constructs are codon optimized for expression in a human cell. In some embodiments, one or more splice donor and/or acceptor sites in a polynucleotide construct is modified to reduce heterogeneity of the RNA transcribed from the construct, such as mRNA, following expression in a cell.

### IV. COMPOSITIONS COMPRISING ENGINEERED CD19 CAR-T CELLS

Provided herein are populations of the engineered T cells, such as containing features or generated by methods described herein, and compositions containing such cells. In some aspects, the provided engineered cells and/or composition of engineered cells include any described herein, e.g., comprising a genetic disruption of the TRAC locus and modification thereof by insertion of a transgene sequence encoding a recombinant CAR, and/or a genetic disruption of the B2M locus and modification thereof by insertion of a transgene sequence encoding a recombinant NK cell inhibitor moiety (e.g., HLA-E fusion protein). In some aspects, the population of engineered cells contain any of the engineered cells described herein, e.g., in Section I herein. In some aspects, the provided cells and cell composition can be engineered using any of the methods described herein, e.g., using agent(s) or methods for introducing genetic disruption, for example, as described in Section I.A herein, and/or using polynucleotides, such as template polynucleotide descried herein, for example in Section I.B, via homology-directed repair (HDR). In some aspects, such cell population is formulated as a pharmaceutical composition or a composition for therapeutic uses or methods.

In some embodiments, the provided compositions contain cells in which cells expressing the recombinant CAR make up at least 30%, 40%, 50%, 60%, 70%, 75%, 80%, 90%, 91%, 92%, 93%, 94%, 95%, 96%, 97%, 98%, 99%, or more of the total cells in the composition, or cells of a certain type, such as T cells or CD8+ or CD4+ cells. In some embodiments, at least 75% or more of the total cells in the composition, or cells of a certain type, such as T cells or CD8+ or CD4+ cells, express the recombinant CAR.

In some embodiments, at least or greater than 5%, 10%, 15%, 20%, 25%, 30%, 35%, 40%, 45%, 50%, 55%, 60%, 65%, 70%, 75%, 80%, 90%, 91%, 92%, 93%, 94%, 95%, 96%, 97%, 98%, 99%, or more of the cells in the composition comprise a genetic disruption at a target site within a gene encoding a domain or region of B2M gene and/or does not express a gene product of an endogenous B2M locus. In some embodiments, at least or greater than 5%, 10%, 15%, 20%, 25%, 30%, 35%, 40%, 45%, 50%, 55%, 60%, 65%, 70%, 75%, 80%, 90%, 91%, 92%, 93%, 94%, 95%, 96%, 97%, 98%, 99%, or more of the cells in the composition does not express a gene product of an endogenous B2M locus. In some embodiments, at least or greater than 95% of the cells in the composition does not express a gene product of an endogenous B2M locus.

In some embodiments, at least or greater than 5%, 10%, 15%, 20%, 25%, 30%, 35%, 40%, 45%, 50%, 55%, 60%, 65%, 70%, 75%, 80%, 90%, 91%, 92%, 93%, 94%, 95%, 96%, 97%, 98%, 99%, or more of the cells in the composition comprise a genetic disruption at a target site within a gene encoding a domain or region of T cell receptor alpha constant (TRAC) gene and/or does not express a gene product of an endogenous TRAC locus. In some embodiments, at least or greater than 35%, 40%, 45%, 50%, 55%, 60%, 65%, 70%, 75%, 80%, 90%, 91%, 92%, 93%, 94%, 95%, 96%, 97%, 98%, 99%, or more of the cells in the composition does not express a gene product of an endogenous TRAC locus. In some embodiments, at least or greater than 95% of the cells in the composition does not express a gene product of an endogenous TRAC locus.

In some embodiments, the provided compositions containing cells in which cells expressing the recombinant HLA-E fusion protein make up at least 30%, 40%, 50%, 60%, 70%, 75%, 80%, 90%, 91%, 92%, 93%, 94%, 95%, 96%, 97%, 98%, 99%, or more of the total cells in the composition, or cells of a certain type, such as T cells or CD8+ or CD4+ cells. In some embodiments, at least 75% or more of the total cells in the composition, or cells of a certain type, such as T cells or CD8+ or CD4+ cells, express the recombinant HLA-E fusion protein.

In some embodiments, at least at or about 50%, 55%, 60%, 65%, 70%, 75%, 80%, 85%, 90%, or 95% of the engineered T cells, or of the total cells or total T cells, in a composition containing a population of engineered T cells express the recombinant CAR and/or exhibits binding to the antigen recognized by the recombinant CAR. In some embodiments, at least at or about 50%, 55%, 60%, 65%, 70%, 75%, 80%, 85%, 90%, or 95% of the engineered T cells, or of the total cells or total T cells, in a composition containing a population of engineered T cells express the recombinant HLA-E fusion protein. In some embodiments, at least at or about 70%, 75%, 80%, 85%, 90%, or 95% of the engineered T cells, or of the total cells or total T cells, in a composition containing a population of engineered T cells do not express a gene product of an endogenous TRAC and/or B2M locus.

In some embodiments, provided are cell populations and/or compositions that include a population of engineered T cells expressing a recombinant CAR and/or HLA-E fusion protein, wherein the nucleic acid sequence encoding the recombinant CAR and/or HLA-E fusion protein is present at the TRAC and/or B2M locus, respectively, e.g., by integration of a transgene encoding recombinant CAR and/or HLA-E fusion protein or a portion thereof at the TRAC and/or B2M locus, respectively, via homology directed repair (HDR).

In some of any embodiments, among a population of cells containing a population of engineered T cells generated by any of the provided methods, greater than about 5%, 10%, 15%, 20%, 25%, 30%, 35%, 40%, 45%, 50%, 60%, 70%, 80%, 90%, 91%, 92%, 93%, 94%, 95%, 96%, 97%, 98%, 99% are knocked out for TRAC (TRAC KO) and/or for B2M (B2M KO), and greater than about 5%, 10%, 15%, 20%, 25%, 30%, 35%, 40%, 45%, 50%, 60%, 70%, 80%, 90%, 91%, 92%, 93%, 94%, 95%, 96%, 97%, 98%, 99% have knock-in of the recombinant CAR and/or greater than about 5%, 10%, 15%, 20%, 25%, 30%, 35%, 40%, 45%, 50%, 60%, 70%, 80%, 90%, 91%, 92%, 93%, 94%, 95%, 96%, 97%, 98%, 99% have knock in of the recombinant HLA-E fusion protein.

In some of any embodiments, among a population of cells containing a population of engineered T cells generated by any of the provided methods greater than 85% are knocked out for TRAC (TRAC KO) and/or for B2M (B2M KO), and greater than 50% have knock-in of the recombinant CAR and/or HLA-E fusion protein. In some of any embodiments, among a population of cells containing a population of engineered T cells generated by any of the provided methods greater than 90% are knocked out for TRAC (TRAC KO) and/or for B2M (B2M KO), and greater than 70% have knock-in of the recombinant CAR and/or HLA-E fusion protein. In some of any embodiments, among a population of cells containing a population of engineered T cells generated by any of the provided methods greater than 95% are knocked out for TRAC (TRAC KO) and/or for B2M (B2M KO), and greater than 75% have knock-in of the recombinant CAR and/or HLA-E fusion protein.

In some embodiments, knock-in of the recombinant CAR and/or HLA-E fusion protein is determined by a PCR-based method, such as ddPCR. In some embodiments, knock-in of the recombinant CAR and/or HLA-E fusion protein is determined by flow cytometry for expression of the recombinant CAR and/or HLA-E fusion protein. In some embodiments, knock-out of the endogenous TRAC or B2M locus is determined molecularly, such as by PCR-based methods (e.g. ddPCR) or next genome sequencing (NGS).

In some embodiments, at least at or about 50%, 60%, 70%, 80%, 90%, 91%, 92%, 93%, 94%, 95%, 96%, 97%, 98%, 99% of the engineered T cells in a composition or a population of T cells express the recombinant CAR and/or HLA-E fusion; and at least at or about 50%, 60%, 70%, 80%, 90%, 91%, 92%, 93%, 94%, 95%, 96%, 97%, 98%, 99% of the engineered T cells in a composition or a population of T cells do not express a gene product of an endogenous TRAC and/or B2M locus.

In some embodiments, at least at or about 50% of the engineered T cells in a composition or a population of T cells express the recombinant CAR and/or HLA-E fusion; and at least at or about 70% of the engineered T cells in a composition or a population of T cells do not express a gene product of an endogenous TRAC and/or B2M locus.

In some embodiments, at least at or about 75% of the engineered T cells, or of the total cells or total T cells, in a composition containing a population of engineered T cells express the recombinant CAR and/or HLA-E fusion; and at least at or about 95% of the engineered T cells, or of the total cells or total T cells, in a composition containing a population of engineered T cells do not express a gene product of an endogenous TRAC and/or B2M locus.

In some embodiments, at least at or about 75% of the total cells or total T cells, in a composition containing a population of engineered T cells express the recombinant CAR and/or HLA-E fusion; and at least at or about 95% of the total cells or total T cells, in a composition containing a population of engineered T cells do not express a gene product of an endogenous TRAC and/or B2M locus.

In some embodiments, the provided cell population and/or compositions containing engineered cells include a cell population that exhibits more improved, uniform, homogeneous and/or stable expression by the recombinant CAR and/or HLA-E fusion, e.g., exhibit reduced coefficient of variation, compared to the expression and/or antigen binding of cell populations and/or compositions generated using other methods. In some embodiments, the cell population and/or compositions exhibit at least 100%, 95%, 90%, 80%, 70%, 60%, 50%, 40%, 30%, 20% or 10% lower coefficient of variation of expression of the recombinant CAR and/or HLA-E fusion protein to a respective population generated using other methods, e.g., random integration of sequences encoding the recombinant CAR and/or HLA-E fusion protein. The coefficient of variation is defined as standard deviation of expression of the nucleic acid of interest (e.g., transgene sequences encoding a recombinant CAR and/or HLA-E fusion or portion thereof) within a population of cells, for example CD4+ and/or CD8+ T cells, divided by the mean of expression of the respective nucleic acid of interest in the respective population of cells. In some embodiments, the cell population and/or compositions exhibit a coefficient of variation that is lower than 0.70, 0.65, 0.60, 0.55, 0.50, 0.45, 0.40, 0.35 or 0.30 or less, when measured among CD4+ and/or CD8+ T cell populations that have been engineered using the methods provided herein.

In some embodiments, the provided cell population and/or compositions containing engineered cells include a cell population that exhibits minimal or reduced random integration of the transgene encoding a recombinant CAR and/or HLA-E fusion protein or a portion thereof. In some aspects, random integration of transgene into the genome of the cell can result in adverse effects or cell death due to integration of the transgene into undesired location in the genome, e.g., into an essential gene or a gene critical in regulating the activity of the cell, and/or unregulated or uncontrolled expression of the receptor. In some aspects, random integration of the transgene is reduced by at least or greater than 50%, 60%, 70%, 80%, 90%, 91%, 92%, 93%, 94%, 95%, 96%, 97%, 98%, 99%, or more compared to cell populations generated using other methods.

In some aspects, the composition of cells comprises CD4+ T cells and/or CD8+ T cells. In some aspects, the composition of cells comprises CD4+ T cells and CD8+ T cells. In some aspects, the percentage of CD4+ T cells in the composition is between at or about 20% and at or about 80%, or at or about 20%, 30%, 40%, 50%, 60%, 70%, or 80% of the total cells in the composition. In some aspects, the percentage of CD8+ T cells in the composition is between at or about 20% and at or about 80%, or at or about 20%, 30%, 40%, 50%, 60%, 70%, or 80% of the total cells in the composition. In some embodiments, the percentage of CD4+ T cells in the composition is between at or about 20% and at or about 80%, or at or about 20%, 30%, 40%, 50%, 60%, 70%, or 80% of the total cells in the composition; and the percentage of CD8+ T cells in the composition is between at or about 20% and at or about 80%, or at or about 20%, 30%, 40%, 50%, 60%, 70%, or 80% of the total cells in the composition. In some aspects, the composition comprises CD4+ T cells and CD8+ T cells, and the ratio of CD4+ T cells to CD8+ T cells is from at or about 1:3 to at or about 3:1. In some aspects, the composition comprises CD4+ T cells and CD8+ T cells, and the ratio of CD4+ T cells to CD8+ T cells is at or about 1:1.

Also provided are compositions, such as pharmaceutical compositions and formulations for administration, containing any of the engineered cells. Such compositions can be used in accord with the provided methods, and/or with the provided articles of manufacture or compositions, such as in the prevention or treatment of diseases, conditions, and disorders, or in detection, diagnostic, and prognostic methods.

Several embodiments relate to compositions comprising a population of CD19 CAR-T cells described herein. In certain embodiments, the T cells are from a healthy donor aged 18 to 35 years old. In certain embodiments, the T cells are from a healthy donor having a body mass index (BMI) less than 30 kg/m². In certain embodiments, the T cells are from a healthy donor between aged 18 to 35 years old and having a body mass index (BMI) less than 30 kg/m². In certain embodiments, the healthy donor is male. In certain embodiments, the healthy donor is female. In certain embodiments, the T cells are from a healthy male donor aged 18 to 35 years old and having a body mass index (BMI) less than 30 kg/m². In certain embodiments, the healthy donor is female. In certain embodiments, the T cells are from a healthy female donor aged 18 to 35 years old and having a body mass index (BMI) less than 30 kg/m². In certain embodiments, the female is nulliparous and non-pregnant. In certain embodiments, the T cells are from a healthy, nulliparous, non-pregnant female donor aged 18 to 35 years old and having a body mass index (BMI) less than 30 kg/m².

In some embodiments, a composition comprises a population of T cells from a healthy donor aged 18 to 35 years old and having a body mass index (BMI) less than 30 kg/m², wherein the T cells comprise (i) a modified TRAC locus comprising a transgene sequence encoding a recombinant CD19 CAR provided herein and (ii) a modified B2M locus comprising a transgene sequence encoding a recombinant HLA-E fusion protein provided herein. In certain embodiments, the healthy donor is male. In certain embodiments, the healthy donor is female. In certain embodiments, the female is nulliparous and non-pregnant. In certain embodiments, the T cells are from a healthy, nulliparous, non-pregnant female donor aged 18 to 35 years old and having a body mass index (BMI) less than 30 kg/m².

In some embodiments, a composition comprises a population of T cells from a healthy donor aged 18 to 35 years old and having a body mass index (BMI) less than 30 kg/m², wherein the T cells comprise (i) a modified TRAC locus comprising a transgene sequence comprising the sequence set forth in SEQ ID NO: 136, and (ii) a modified B2M locus comprising a transgene sequence comprising the sequence set forth in SEQ ID NO: 86. In certain embodiments, the healthy donor is male. In certain embodiments, the healthy donor is female. In certain embodiments, the female is nulliparous and non-pregnant. In certain embodiments, the T cells are from a healthy, nulliparous, non-pregnant female donor aged 18 to 35 years old and having a body mass index (BMI) less than 30 kg/m². In certain embodiments, the population of T cells is from a single healthy donor.

In some embodiments, a composition comprises a population of T cells from a healthy donor aged 18 to 35 years old and having a body mass index (BMI) less than 30 kg/m², wherein the T cells comprise (i) a modified TRAC locus comprising a transgene sequence comprising the sequence set forth in SEQ ID NO: 94, and (ii) a modified B2M locus comprising a transgene sequence comprising the sequence set forth in SEQ ID NO: 137. In certain embodiments, the healthy donor is male. In certain embodiments, the healthy donor is female. In certain embodiments, the female is nulliparous and non-pregnant. In certain embodiments, the T cells are from a healthy, nulliparous, non-pregnant female donor aged 18 to 35 years old and having a body mass index (BMI) less than 30 kg/m². In certain embodiments, the population of T cells is from a single healthy donor.

In some embodiments, a composition comprises a population of T cells from a healthy donor aged 18 to 35 years old and having a body mass index (BMI) less than 30 kg/m², wherein the T cells comprise (i) a modified TRAC locus comprising a transgene sequence encoding a CD19 CAR having an amino acid sequence set forth in SEQ ID NO: 138, and (ii) a modified B2M locus comprising a transgene sequence comprising the sequence set forth in SEQ ID NO: 132. In certain embodiments, the healthy donor is male. In certain embodiments, the healthy donor is female. In certain embodiments, the female is nulliparous and non-pregnant. In certain embodiments, the T cells are from a healthy, nulliparous, non-pregnant female donor aged 18 to 35 years old and having a body mass index (BMI) less than 30 kg/m². In certain embodiments, the population of T cells is from a single healthy donor.

In some embodiments, a composition comprises a population of T cells from a healthy donor aged 18 to 35 years old and having a body mass index (BMI) less than 30 kg/m², wherein the T cells comprise (i) a modified TRAC locus comprising a transgene sequence encoding a CD19 CAR having an amino acid sequence set forth in SEQ ID NO: 78, and (ii) a modified B2M locus comprising a transgene sequence comprising the sequence set forth in SEQ ID NO: 81. In certain embodiments, the healthy donor is male. In certain embodiments, the healthy donor is female. In certain embodiments, the female is nulliparous and non-pregnant. In certain embodiments, the T cells are from a healthy, nulliparous, non-pregnant female donor aged 18 to 35 years old and having a body mass index (BMI) less than 30 kg/m². In certain embodiments, the population of T cells is from a single healthy donor.

In certain embodiments, a composition comprises a population of T cells wherein the composition has one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, twenty-four or more, twenty-five or more, twenty-six or more, twenty-seven or more, or all the following attributes:
(i) at least about 50%, at least about 55%, at least about 60%, at least about 65%, at least about 70%, at least about 75%, at least about 80%, at least about 85%, at least about 90%, or at least about 95%, at least about 96%, at least about 97%, at least about 98%, at least about 99%, or 100% of the T cells are viable;
(ii) at least about 70%, at least about 75%, at least about 80%, at least about 85%, at least about 90%, at least about 95%, at least about 96%, at least about 97%, at least about 98%, at least about 99%, or 100% of the T cells are CD2+CD5+;
(iii) at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, at least about 96%, at least about 97%, at least about 98%, at least about 99%, or 100% of the T cells express CD19 CAR, such as the CD19 CAR having an amino acid sequence set forth in SEQ ID NO: 78 or SEQ ID NO: 138;
(iv) less than about 1%, less than about 2%, less than about 3%, less than about 4%, less than about 5%, less than about 6%, less than about 7%, less than about 8%, less than about 9%, or less than about 10% of the T cells are HLA-I+;
(v) the ratio of % TCR+ to % CAR+ T cells is less than about 0.005, less than about 0.006, less than about 0.007, less than about 0.008, less than about 0.009, or less than about 0.01;
(vi) less than about 1% of total alleles, less than about 2% of total alleles, less than about 3% of total alleles, less than about 4% of total alleles, less than about 5% of total alleles, less than about 6% of total alleles, less than about 7% of total alleles, less than about 8% of total alleles, less than about 9% of total alleles, or less than about 10% of total alleles have translocation between TRAC and B2M;
(vii) at least about 5,000,000, at least about 10,000,000, at least about 15,000,000, at least about 20,000,000, at least about 25,000,000, at least about 30,000,000, at least about 35,000,000, at least about 40,000,000, at least about 45,000,000, or at least about 50,000,000 viable CD2+CD5+CD19 CAR+ cells per mL of the composition;
(viii) the T cells are from a healthy donor, such as a single healthy donor, aged 18 to 35 years old;
(ix) the T cells are from a healthy donor, such as a single healthy donor, having a body mass index (BMI) less than 30 kg/m²;
(x) at least about 70% of total alleles, at least about 75% of total alleles, at least about 80% of total alleles, at least about 85% of total alleles, at least about 90% of total alleles, at least about 95% of total alleles, at least about 96% of total alleles, at least about 97% of total alleles, at least about 98% of total alleles, at least about 99% of total alleles, or 100% of total alleles have edited TRAC loci;
(xi) less than about 0.1%, less than about 0.2%, less than about 0.3%, less than about 0.4%, less than about 0.5%, less than about 0.6%, less than about 0.7%, less than about 0.8%, less than about 0.9%, or less than about 1% of the T cells are TCR+ T cells;
(xii) at least about 70% of total alleles, at least about 75% of total alleles, at least about 80% of total alleles, at least about 85% of total alleles, at least about 90% of total alleles, at least about 95% of total alleles, at least about 96% of total alleles, at least about 97% of total alleles, at least about 98% of total alleles, at least about 99% of total alleles, or 100% of total alleles have edited B2M loci;
(xiii) at least about 25% of total alleles, at least about 30% of total alleles, at least about 35% of total alleles, at least about 40% of total alleles, at least about 45% of total alleles, at least about 50% of total alleles, at least about 55% of total alleles, at least about 60% of total alleles, at least about 65% of total alleles, at least about 70% of total alleles, at least about 75% of total alleles, at least about 80% of total alleles, at least about 85% of total alleles, at least about 90% of total alleles, at least about 95% of total alleles, at least about 96% of total alleles, at least about 97% of total alleles, at least about 98% of total alleles, at least about 99% of total alleles, or 100% of total alleles have a nucleic acid encoding HLA-E(A) knocked-in at the B2M locus, such as a nucleic acid comprising the sequence set forth in SEQ ID NO: 86, or a nucleic acid encoding HLA-E(A) having an amino acid sequence set forth in SEQ ID NO: 81 or SEQ ID NO: 132;
(xiv) at least about 25% of total alleles, at least about 30% of total alleles, at least about 35% of total alleles, at least about 40% of total alleles, at least about 45% of total alleles, at least about 50% of total alleles, at least about 55% of total alleles, at least about 60% of total alleles, at least about 65% of total alleles, at least about 70% of total alleles, at least about 75% of total alleles, at least about 80% of total alleles, at least about 85% of total alleles, at least about 90% of total alleles, at least about 95% of total alleles, at least about 96% of total alleles, at least about 97% of total alleles, at least about 98% of total alleles, at least about 99% of total alleles, or 100% of total alleles have a nucleic acid encoding CD19 CAR knocked-in at the TRAC locus, such as a nucleic acid comprising the sequence set forth in SEQ ID NO: 136, or a nucleic acid encoding a CD19 CAR having an amino acid sequence set forth in SEQ ID NO: 78 or SEQ ID NO: 138;
(xv) less than about 1 ng, less than about 2 ng, less than about 3 ng, less than about 4 ng, less than about 5 ng, less than about 6 ng, less than about 7 ng, less than about 8 ng, less than about 9 ng, less than about 10 ng, less than about 11 ng, less than about 12 ng, less than about 13 ng, less than about 14 ng, less than about 15 ng, less than about 16 ng, less than about 17 ng, less than about 18 ng, less than about 19 ng, or less than about 20 ng of Cas9 or Cas12a per mL of the composition;
(xvi) less than about 0.01 fg, less than about 0.02 fg, less than about 0.03 fg, less than about 0.04 fg, less than about 0.05 fg, less than about 0.06 fg, less than about 0.07 fg, less than about 0.08 fg, less than about 0.09 fg, or less than about 1.0 fg of Cas9 or Cas12a per T cell;
(xvii) less than about 1×10¹⁰, less than about 5×10¹⁰, less than about 1×10¹¹, less than about 5×10¹¹, or less than about 1×10¹² AAV6 capsids per mL of the composition;
(xviii) ten or fewer, nine or fewer, eight or fewer, seven or fewer, six or fewer, five or fewer, four or fewer, three or fewer, two or fewer, one or fewer, or no verified off-target indel sites at a genomic locus other than TRAC and B2M;
(xix) less than about 0.5% of total alleles, less than about 1.0% of total alleles, less than about 1.5% of total alleles, less than about 2.0% of total alleles, less than about 2.5% of total alleles, less than about 3.0% of total alleles, less than about 3.5% of total alleles, or less than about 4.0% of total alleles have AAV6 integration at TRAC locus;
(xx) less than about 0.5% of total alleles, less than about 0.6% of total alleles, less than about 0.7% of total alleles, less than about 0.8% of total alleles, less than about 0.9% of total alleles, less than about 1.0% of total alleles, less than about 1.1% of total alleles, less than about 1.2% of total alleles, less than about 1.3% of total alleles, less than about 1.4% of total alleles, less than about 1.5% of total alleles, less than about 1.6% of total alleles, less than about 1.7% of total alleles, less than about 1.8% of total alleles, less than about 1.9% of total alleles, or less than about 2.0% of total alleles have AAV6 integration at B2M locus;
(xxi) undetectable AAV6 integration at loci other than TRAC and B2M;
(xxii) less than about 100 ng, less than about 150 ng, less than about 200 ng, less than about 250 ng, less than about 300 ng, less than about 350 ng, less than about 400 ng, less than about 450 ng, less than about 500 ng, less than about 550 ng, less than about 600 ng, less than about 650 ng, less than about 700 ng, less than about 750 ng, less than about 800 ng, less than about 850 ng, less than about 900 ng, less than about 950 ng, less than about 1000 ng, less than about 1100 ng, less than about 1200 ng, less than about 1300 ng, less than about 1400 ng, or less than about 1500 ng of Strep-Tactin Multimer activation reagent per mL of the composition;
(xxiii) no detected bacterial growth;
(xxiv) no more than about 10 EU/mL, no more than about 11 EU/mL, no more than about 12 EU/mL, no more than about 13 EU/mL, no more than about 14 EU/mL, or no more than about 15 EU/mL endotoxin;
(xxv) no detected mycoplasma
(xxvi) no cytokine-independent growth;
(xxvii) no significant unexpected karyotype; or
(xxviii) negative for the presence of HIV, HTLV, HAV, HBV, HCV, CMV, EBV, HHV6, HHV7, HHV8, and B19.

In certain embodiments, a composition comprises a population of T cells wherein the composition has one or more, two or more, three or more, four or more, five or more, six or more, seven or more, eight or more, nine or more, ten or more, eleven or more, twelve or more, thirteen or more, fourteen or more, or all the following attributes:
(i) at least about 50%, at least about 55%, at least about 60%, at least about 65%, at least about 70%, at least about 75%, at least about 80%, at least about 85%, at least about 90%, or at least about 95%, at least about 96%, at least about 97%, at least about 98%, at least about 99%, or 100% of the T cells are viable;
(ii) at least about 70%, at least about 75%, at least about 80%, at least about 85%, at least about 90%, at least about 95%, at least about 96%, at least about 97%, at least about 98%, at least about 99%, or 100% of the T cells are CD2+CD5+;
(iii) at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, at least about 96%, at least about 97%, at least about 98%, at least about 99%, or 100% of the T cells express CD19 CAR, such as the CD19 CAR having an amino acid sequence set forth in SEQ ID NO: 78 or SEQ ID NO: 138;
(iv) less than about 1%, less than about 2%, less than about 3%, less than about 4%, less than about 5%, less than about 6%, less than about 7%, less than about 8%, less than about 9%, or less than about 10% of the T cells are HLA-I+;
(v) the ratio of % TCR+ to % CAR+ T cells is less than about 0.005, less than about 0.006, less than about 0.007, less than about 0.008, less than about 0.009, or less than about 0.01;
(vi) less than about 1% of total alleles, less than about 2% of total alleles, less than about 3% of total alleles, less than about 4% of total alleles, less than about 5% of total alleles, less than about 6% of total alleles, less than about 7% of total alleles, less than about 8% of total alleles, less than about 9% of total alleles, or less than about 10% of total alleles have translocation between TRAC and B2M;
(vii) at least about 5,000,000, at least about 10,000,000, at least about 15,000,000, at least about 20,000,000, at least about 25,000,000, at least about 30,000,000, at least about 35,000,000, at least about 40,000,000, at least about 45,000,000, or at least about 50,000,000 viable CD2+CD5+CD19 CAR+ cells per mL of the composition;
(viii) no detected bacterial growth;
(ix) no more than about 10 EU/mL, no more than about 11 EU/mL, no more than about 12 EU/mL, no more than about 13 EU/mL, no more than about 14 EU/mL, or no more than about 15 EU/mL endotoxin;
(x) no detected mycoplasma;
(xi) no cytokine-independent growth;
(xii) no significant unexpected karyotype;
(xiii) negative for the presence of HIV, HTLV, HAV, HBV, HCV, CMV, EBV, HHV6, HHV7, HHV8, and B19.
(xiv) the T cells are from a healthy donor, such as a single healthy donor, aged 18 to 35 years old; or
(xv) the T cells are from a healthy donor, such as a single healthy donor, having a body mass index (BMI) less than 30 kg/m².

In certain embodiments, a composition comprises a population of T cells wherein the T cells are from a healthy donor, such as a single healthy donor, aged 18 to 35 years old and having a body mass index (BMI) less than 30 kg/m², and wherein the composition has one or more, two or more, three or more, four or more, five or more, six or more, seven or more, eight or more, nine or more, ten or more, eleven or more, twelve or more, or all the following attributes:
(i) at least about 50%, at least about 55%, at least about 60%, at least about 65%, at least about 70%, at least about 75%, at least about 80%, at least about 85%, at least about 90%, or at least about 95%, at least about 96%, at least about 97%, at least about 98%, at least about 99%, or 100% of the T cells are viable;
(ii) at least about 70%, at least about 75%, at least about 80%, at least about 85%, at least about 90%, at least about 95%, at least about 96%, at least about 97%, at least about 98%, at least about 99%, or 100% of the T cells are CD2+CD5+;
(iii) at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, at least about 96%, at least about 97%, at least about 98%, at least about 99%, or 100% of the T cells express CD19 CAR, such as the CD19 CAR having an amino acid sequence set forth in SEQ ID NO: 78 or SEQ ID NO: 138;
(iv) less than about 1%, less than about 2%, less than about 3%, less than about 4%, less than about 5%, less than about 6%, less than about 7%, less than about 8%, less than about 9%, or less than about 10% of the T cells are HLA-I+;
(v) the ratio of % TCR+ to % CAR+ T cells is less than about 0.005, less than about 0.006, less than about 0.007, less than about 0.008, less than about 0.009, or less than about 0.01;
(vi) less than about 1% of total alleles, less than about 2% of total alleles, less than about 3% of total alleles, less than about 4% of total alleles, less than about 5% of total alleles, less than about 6% of total alleles, less than about 7% of total alleles, less than about 8% of total alleles, less than about 9% of total alleles, or less than about 10% of total alleles have translocation between TRAC and B2M;
(vii) at least about 5,000,000, at least about 10,000,000, at least about 15,000,000, at least about 20,000,000, at least about 25,000,000, at least about 30,000,000, at least about 35,000,000, at least about 40,000,000, at least about 45,000,000, or at least about 50,000,000 viable CD2+CD5+CD19 CAR+ cells per mL of the composition;
(viii) no detected bacterial growth;
(ix) no more than about 10 EU/mL, no more than about 11 EU/mL, no more than about 12 EU/mL, no more than about 13 EU/mL, no more than about 14 EU/mL, or no more than about 15 EU/mL endotoxin;
(x) no detected mycoplasma;
(xi) no cytokine-independent growth;
(xii) no significant unexpected karyotype; or
(xiii) negative for the presence of HIV, HTLV, HAV, HBV, HCV, CMV, EBV, HHV6, HHV7, HHV8, and B19.

In certain embodiments, a composition comprises a population of T cells wherein the T cells are from a single healthy donor aged 18 to 35 years old and having a body mass index (BMI) less than 30 kg/m², and wherein the composition has one or more, two or more, three or more, four or more, five or more, six or more, seven or more, eight or more, nine or more, ten or more, eleven or more, twelve or more, or all the following attributes:
(i) at least about 50%, at least about 55%, at least about 60%, at least about 65%, at least about 70%, at least about 75%, at least about 80%, at least about 85%, at least about 90%, or at least about 95%, at least about 96%, at least about 97%, at least about 98%, at least about 99%, or 100% of the T cells are viable;
(ii) at least about 70%, at least about 75%, at least about 80%, at least about 85%, at least about 90%, at least about 95%, at least about 96%, at least about 97%, at least about 98%, at least about 99%, or 100% of the T cells are CD2+CD5+;
(iii) at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, at least about 96%, at least about 97%, at least about 98%, at least about 99%, or 100% of the T cells express CD19 CAR, such as the CD19 CAR having an amino acid sequence set forth in SEQ ID NO: 78 or SEQ ID NO: 138;
(iv) less than about 1%, less than about 2%, less than about 3%, less than about 4%, less than about 5%, less than about 6%, less than about 7%, less than about 8%, less than about 9%, or less than about 10% of the T cells are HLA-I+;
(v) the ratio of % TCR+ to % CAR+ T cells is less than about 0.005, less than about 0.006, less than about 0.007, less than about 0.008, less than about 0.009, or less than about 0.01;
(vi) less than about 1% of total alleles, less than about 2% of total alleles, less than about 3% of total alleles, less than about 4% of total alleles, less than about 5% of total alleles, less than about 6% of total alleles, less than about 7% of total alleles, less than about 8% of total alleles, less than about 9% of total alleles, or less than about 10% of total alleles have translocation between TRAC and B2M;
(vii) at least about 5,000,000, at least about 10,000,000, at least about 15,000,000, at least about 20,000,000, at least about 25,000,000, at least about 30,000,000, at least about 35,000,000, at least about 40,000,000, at least about 45,000,000, or at least about 50,000,000 viable CD2+CD5+CD19 CAR+ cells per mL of the composition;
(viii) no detected bacterial growth;
(ix) no more than about 10 EU/mL, no more than about 11 EU/mL, no more than about 12 EU/mL, no more than about 13 EU/mL, no more than about 14 EU/mL, or no more than about 15 EU/mL endotoxin;
(x) no detected mycoplasma;
(xi) no cytokine-independent growth;
(xii) no significant unexpected karyotype; or
(xiii) negative for the presence of HIV, HTLV, HAV, HBV, HCV, CMV, EBV, HHV6, HHV7, HHV8, and B19, and wherein the composition has one or more of the following attributes:
(a) at least about 70% of total alleles, at least about 75% of total alleles, at least about 80% of total alleles, at least about 85% of total alleles, at least about 90% of total alleles, at least about 95% of total alleles, at least about 96% of total alleles, at least about 97% of total alleles, at least about 98% of total alleles, at least about 99% of total alleles, or 100% of total alleles have edited TRAC loci;
(b) less than about 0.1%, less than about 0.2%, less than about 0.3%, less than about 0.4%, less than about 0.5%, less than about 0.6%, less than about 0.7%, less than about 0.8%, less than about 0.9%, or less than about 1% of the T cells are TCR+ T cells;
(c) at least about 70% of total alleles, at least about 75% of total alleles, at least about 80% of total alleles, at least about 85% of total alleles, at least about 90% of total alleles, at least about 95% of total alleles, at least about 96% of total alleles, at least about 97% of total alleles, at least about 98% of total alleles, at least about 99% of total alleles, or 100% of total alleles have edited B2M loci;
(d) at least about 25% of total alleles, at least about 30% of total alleles, at least about 35% of total alleles, at least about 40% of total alleles, at least about 45% of total alleles, at least about 50% of total alleles, at least about 55% of total alleles, at least about 60% of total alleles, at least about 65% of total alleles, at least about 70% of total alleles, at least about 75% of total alleles, at least about 80% of total alleles, at least about 85% of total alleles, at least about 90% of total alleles, at least about 95% of total alleles, at least about 96% of total alleles, at least about 97% of total alleles, at least about 98% of total alleles, at least about 99% of total alleles, or 100% of total alleles have a nucleic acid encoding HLA-E(A) knocked-in at the B2M locus, such as a nucleic acid comprising the sequence set forth in SEQ ID NO: 86, or a nucleic acid encoding HLA-E(A) having an amino acid sequence set forth in SEQ ID NO: 81 or SEQ ID NO: 132;
(e) at least about 25% of total alleles, at least about 30% of total alleles, at least about 35% of total alleles, at least about 40% of total alleles, at least about 45% of total alleles, at least about 50% of total alleles, at least about 55% of total alleles, at least about 60% of total alleles, at least about 65% of total alleles, at least about 70% of total alleles, at least about 75% of total alleles, at least about 80% of total alleles, at least about 85% of total alleles, at least about 90% of total alleles, at least about 95% of total alleles, at least about 96% of total alleles, at least about 97% of total alleles, at least about 98% of total alleles, at least about 99% of total alleles, or 100% of total alleles have a nucleic acid encoding CD19 CAR knocked-in at the TRAC locus, such as a nucleic acid comprising the sequence set forth in SEQ ID NO: 136, or a nucleic acid encoding a CD19 CAR having an amino acid sequence set forth in SEQ ID NO: 78 or SEQ ID NO: 138;
(f) less than about 1 ng, less than about 2 ng, less than about 3 ng, less than about 4 ng, less than about 5 ng, less than about 6 ng, less than about 7 ng, less than about 8 ng, less than about 9 ng, less than about 10 ng, less than about 11 ng, less than about 12 ng, less than about 13 ng, less than about 14 ng, less than about 15 ng, less than about 16 ng, less than about 17 ng, less than about 18 ng, less than about 19 ng, or less than about 20 ng of Cas9 or Cas12a per mL of the composition;
(g) less than about 0.01 fg, less than about 0.02 fg, less than about 0.03 fg, less than about 0.04 fg, less than about 0.05 fg, less than about 0.06 fg, less than about 0.07 fg, less than about 0.08 fg, less than about 0.09 fg, or less than about 1.0 fg of Cas9 or Cas12a per T cell;
(h) less than about 1×10¹⁰, less than about 5×10¹⁰, less than about 1×10¹¹, less than about 5×10¹¹, or less than about 1×10¹² AAV6 capsids per mL of the composition;
(i) ten or fewer, nine or fewer, eight or fewer, seven or fewer, six or fewer, five or fewer, four or fewer, three or fewer, two or fewer, one or fewer, or no verified off-target indel sites at a genomic locus other than TRAC and B2M;
(j) less than about 0.5% of total alleles, less than about 1.0% of total alleles, less than about 1.5% of total alleles, less than about 2.0% of total alleles, less than about 2.5% of total alleles, less than about 3.0% of total alleles, less than about 3.5% of total alleles, or less than about 4.0% of total alleles have AAV6 integration at TRAC locus;
(k) less than about 0.5% of total alleles, less than about 0.6% of total alleles, less than about 0.7% of total alleles, less than about 0.8% of total alleles, less than about 0.9% of total alleles, less than about 1.0% of total alleles, less than about 1.1% of total alleles, less than about 1.2% of total alleles, less than about 1.3% of total alleles, less than about 1.4% of total alleles, less than about 1.5% of total alleles, less than about 1.6% of total alleles, less than about 1.7% of total alleles, less than about 1.8% of total alleles, less than about 1.9% of total alleles, or less than about 2.0% of total alleles have AAV6 integration at B2M locus;
(l) undetectable AAV6 integration at loci other than TRAC and B2M; or
(m) less than about 100 ng, less than about 150 ng, less than about 200 ng, less than about 250 ng, less than about 300 ng, less than about 350 ng, less than about 400 ng, less than about 450 ng, less than about 500 ng, less than about 550 ng, less than about 600 ng, less than about 650 ng, less than about 700 ng, less than about 750 ng, less than about 800 ng, less than about 850 ng, less than about 900 ng, less than about 950 ng, less than about 1000 ng, less than about 1100 ng, less than about 1200 ng, less than about 1300 ng, less than about 1400 ng, or less than about 1500 ng of Strep-Tactin Multimer activation reagent per mL of the composition.

In certain embodiments, a composition comprises a population of T cells wherein the composition has one or more, two or more, three or more, four or more, five or more, six or more, seven or more, eight or more, nine or more, ten or more, eleven or more, twelve or more, thirteen or more, fourteen or more, or all the following attributes:
(i) at least about 70% of the T cells are viable;
(ii) at least about 90% of the T cells are CD2+CD5+;
(iii) at least about 50% of the T cells express CD19 CAR, such as the CD19 CAR having an amino acid sequence set forth in SEQ ID NO: 78 or SEQ ID NO: 138;
(iv) less than about 5% of the T cells are HLA-I+;
(v) the ratio of % TCR+ to % CAR+ T cells is less than about 0.008;
(vi) less than about 5% of total alleles have translocation between TRAC and B2M; or
(vii) at least about 20,000,000 viable CD2+CD5+CD19 CAR+ cells per mL of the composition;
(viii) the T cells are from a healthy donor, such as a single healthy donor, aged 18 to 35 years old;
(ix) the T cells are from a healthy donor, such as a single healthy donor, having a body mass index (BMI) less than 30 kg/m²;
(x) no detected bacterial growth;
(xi) no more than about 13.3 EU/mL endotoxin;
(xii) no detected mycoplasma;
(xiii) no cytokine-independent growth;
(xiv) no significant unexpected karyotype;
(xv) negative for the presence of HIV, HTLV, HAV, HBV, HCV, CMV, EBV, HHV6, HHV7, HHV8, and B19.

In certain embodiments, a composition comprises a population of T cells, wherein the T cells are from a single healthy donor aged 18 to 35 years old and having a body mass index (BMI) less than 30 kg/m², and wherein the composition has one or more, two or more, three or more, four or more, five or more, six or more, seven or more, eight or more, nine or more, ten or more, eleven or more, twelve or more, or all the following attributes:
(i) at least about 70% of the T cells are viable;
(ii) at least about 90% of the T cells are CD2+CD5+;
(iii) at least about 50% of the T cells express CD19 CAR, such as the CD19 CAR having an amino acid sequence set forth in SEQ ID NO: 78 or SEQ ID NO: 138.
(iv) less than about 5% of the T cells are HLA-I+;
(v) the ratio of % TCR+ to % CAR+ T cells is less than about 0.008;
(vi) less than about 5% of total alleles have translocation between TRAC and B2M;
(vii) at least about 20,000,000 viable CD2+CD5+CD19 CAR+ cells per mL of the composition;
(viii) no detected bacterial growth;
(ix) no more than about 13.3 EU/mL endotoxin;
(x) no detected mycoplasma;
(xi) no cytokine-independent growth;
(xii) no significant unexpected karyotype; or
(xiii) negative for the presence of HIV, HTLV, HAV, HBV, HCV, CMV, EBV, HHV6, HHV7, HHV8, and B19.

In certain embodiments, a composition comprises a population of T cells, wherein the T cells are from a single healthy donor aged 18 to 35 years old and having a body mass index (BMI) less than 30 kg/m², and wherein the composition has one or more, two or more, three or more, four or more, five or more, six or more, seven or more, eight or more, nine or more, ten or more, eleven or more, twelve or more, or all the following attributes:
(i) at least about 70% of the T cells are viable;
(ii) at least about 90% of the T cells are CD2+CD5+;
(iii) at least about 50% of the T cells express CD19 CAR, such as the CD19 CAR having an amino acid sequence set forth in SEQ ID NO: 78 or SEQ ID NO: 138;
(iv) less than about 5% of the T cells are HLA-I+;
(v) the ratio of % TCR+ to % CAR+ T cells is less than about 0.008;
(vi) less than about 5% of total alleles have translocation between TRAC and B2M;
(vii) at least about 20,000,000 viable CD2+CD5+CD19 CAR+ cells per mL of the composition;
(viii) no detected bacterial growth;
(ix) no more than about 13.3 EU/mL endotoxin;
(x) no detected mycoplasma;
(xi) no cytokine-independent growth;
(xii) no significant unexpected karyotype; or
(xiii) negative for the presence of HIV, HTLV, HAV, HBV, HCV, CMV, EBV, HHV6, HHV7, HHV8, and B19, and wherein the composition has one or more of the following attributes:
(a) at least about 70% of total alleles, at least about 75% of total alleles, at least about 80% of total alleles, at least about 85% of total alleles, at least about 90% of total alleles, at least about 95% of total alleles, at least about 96% of total alleles, at least about 97% of total alleles, at least about 98% of total alleles, at least about 99% of total alleles, or 100% of total alleles have edited TRAC loci;
(b) less than about 0.1%, less than about 0.2%, less than about 0.3%, less than about 0.4%, less than about 0.5%, less than about 0.6%, less than about 0.7%, less than about 0.8%, less than about 0.9%, or less than about 1% of the T cells are TCR+ T cells;
(c) at least about 70% of total alleles, at least about 75% of total alleles, at least about 80% of total alleles, at least about 85% of total alleles, at least about 90% of total alleles, at least about 95% of total alleles, at least about 96% of total alleles, at least about 97% of total alleles, at least about 98% of total alleles, at least about 99% of total alleles, or 100% of total alleles have edited B2M loci;
(d) at least about 25% of total alleles, at least about 30% of total alleles, at least about 35% of total alleles, at least about 40% of total alleles, at least about 45% of total alleles, at least about 50% of total alleles, at least about 55% of total alleles, at least about 60% of total alleles, at least about 65% of total alleles, at least about 70% of total alleles, at least about 75% of total alleles, at least about 80% of total alleles, at least about 85% of total alleles, at least about 90% of total alleles, at least about 95% of total alleles, at least about 96% of total alleles, at least about 97% of total alleles, at least about 98% of total alleles, at least about 99% of total alleles, or 100% of total alleles have a nucleic acid encoding HLA-E(A) knocked-in at the B2M locus, such as a nucleic acid comprising the sequence set forth in SEQ ID NO: 86, or a nucleic acid encoding HLA-E(A) having an amino acid sequence set forth in SEQ ID NO: 81 or SEQ ID NO: 132;
(e) at least about 25% of total alleles, at least about 30% of total alleles, at least about 35% of total alleles, at least about 40% of total alleles, at least about 45% of total alleles, at least about 50% of total alleles, at least about 55% of total alleles, at least about 60% of total alleles, at least about 65% of total alleles, at least about 70% of total alleles, at least about 75% of total alleles, at least about 80% of total alleles, at least about 85% of total alleles, at least about 90% of total alleles, at least about 95% of total alleles, at least about 96% of total alleles, at least about 97% of total alleles, at least about 98% of total alleles, at least about 99% of total alleles, or 100% of total alleles have a nucleic acid encoding CD19 CAR knocked-in at the TRAC locus, such as a nucleic acid comprising the sequence set forth in SEQ ID NO: 136, or a nucleic acid encoding a CD19 CAR having an amino acid sequence set forth in SEQ ID NO: 78 or SEQ ID NO: 138;
(f) less than about 1 ng, less than about 2 ng, less than about 3 ng, less than about 4 ng, less than about 5 ng, less than about 6 ng, less than about 7 ng, less than about 8 ng, less than about 9 ng, less than about 10 ng, less than about 11 ng, less than about 12 ng, less than about 13 ng, less than about 14 ng, less than about 15 ng, less than about 16 ng, less than about 17 ng, less than about 18 ng, less than about 19 ng, or less than about 20 ng of Cas9 or Cas12a per mL of the composition;
(g) less than about 0.01 fg, less than about 0.02 fg, less than about 0.03 fg, less than about 0.04 fg, less than about 0.05 fg, less than about 0.06 fg, less than about 0.07 fg, less than about 0.08 fg, less than about 0.09 fg, or less than about 1.0 fg of Cas9 or Cas12a per T cell;
(h) less than about 1×10¹⁰, less than about 5×10¹⁰, less than about 1×10¹¹, less than about 5×10¹¹, or less than about 1×10¹² AAV6 capsids per mL of the composition;
(i) ten or fewer, nine or fewer, eight or fewer, seven or fewer, six or fewer, five or fewer, four or fewer, three or fewer, two or fewer, one or fewer, or no verified off-target indel sites at a genomic locus other than TRAC and B2M;
(j) less than about 0.5% of total alleles, less than about 1.0% of total alleles, less than about 1.5% of total alleles, less than about 2.0% of total alleles, less than about 2.5% of total alleles, less than about 3.0% of total alleles, less than about 3.5% of total alleles, or less than about 4.0% of total alleles have AAV6 integration at TRAC locus;
(k) less than about 0.5% of total alleles, less than about 0.6% of total alleles, less than about 0.7% of total alleles, less than about 0.8% of total alleles, less than about 0.9% of total alleles, less than about 1.0% of total alleles, less than about 1.1% of total alleles, less than about 1.2% of total alleles, less than about 1.3% of total alleles, less than about 1.4% of total alleles, less than about 1.5% of total alleles, less than about 1.6% of total alleles, less than about 1.7% of total alleles, less than about 1.8% of total alleles, less than about 1.9% of total alleles, or less than about 2.0% of total alleles have AAV6 integration at B2M locus;
(l) undetectable AAV6 integration at loci other than TRAC and B2M; or
(m) less than about 100 ng, less than about 150 ng, less than about 200 ng, less than about 250 ng, less than about 300 ng, less than about 350 ng, less than about 400 ng, less than about 450 ng, less than about 500 ng, less than about 550 ng, less than about 600 ng, less than about 650 ng, less than about 700 ng, less than about 750 ng, less than about 800 ng, less than about 850 ng, less than about 900 ng, less than about 950 ng, less than about 1000 ng, less than about 1100 ng, less than about 1200 ng, less than about 1300 ng, less than about 1400 ng, or less than about 1500 ng of Strep-Tactin Multimer activation reagent per mL of the composition.

In certain embodiments, a composition comprises a population of T cells, wherein the T cells are from a single healthy donor aged 18 to 35 years old and having a body mass index (BMI) less than 30 kg/m², and wherein the composition has one or more, two or more, three or more, four or more, five or more, six or more, seven or more, eight or more, nine or more, ten or more, eleven or more, twelve or more, or all the following attributes:
(i) at least about 70% of the T cells are viable;
(ii) at least about 90% of the T cells are CD2+CD5+;
(iii) at least about 50% of the T cells express CD19 CAR, such as the CD19 CAR having an amino acid sequence set forth in SEQ ID NO: 78 or SEQ ID NO: 138;
(iv) less than about 5% of the T cells are HLA-I+;
(v) the ratio of % TCR+ to % CAR+ T cells is less than about 0.008;
(vi) less than about 5% of total alleles have translocation between TRAC and B2M;
(vii) at least about 20,000,000 viable CD2+CD5+CD19 CAR+ cells per mL of the composition;
(viii) no detected bacterial growth;
(ix) no more than about 13.3 EU/mL endotoxin;
(x) no detected mycoplasma;
(xi) no cytokine-independent growth;
(xii) no significant unexpected karyotype; or
(xiii) negative for the presence of HIV, HTLV, HAV, HBV, HCV, CMV, EBV, HHV6, HHV7, HHV8, and B19, and wherein the composition has one or more of the following attributes:
(a) at least about 90% of total alleles have edited TRAC loci;
(b) less than about 0.5% of the T cells are TCR+ T cells;
(c) at least about 90% of total alleles have edited B2M loci;
(d) at least about 50% of total alleles have a nucleic acid encoding HLA-E(A) knocked-in at the B2M locus, such as a nucleic acid comprising the sequence set forth in SEQ ID NO: 86, or a nucleic acid encoding HLA-E(A) having an amino acid sequence set forth in SEQ ID NO: 81 or SEQ ID NO: 132;
(e) at least about 50% of total alleles have a nucleic acid encoding CD19 CAR knocked-in at the TRAC locus, such as a nucleic acid comprising the sequence set forth in SEQ ID NO: 136, or a nucleic acid encoding a CD19 CAR having an amino acid sequence set forth in SEQ ID NO: 78 or SEQ ID NO: 138;
(f) less than about 2 ng of Cas9 or Cas12a per mL of the composition;
(g) less than about 0.05 fg of Cas9 or Cas12a per T cell;
(h) less than about 5×10¹¹ AAV6 capsids per mL of the composition;
(i) no verified off-target indel sites at a genomic locus other than TRAC and B2M;
(j) less than about 2.0% of total alleles have AAV6 integration at TRAC locus;
(k) less than about 1.0% of total alleles have AAV6 integration at B2M locus;
(l) undetectable AAV6 integration at loci other than TRAC and B2M; or
(m) less than about 900 ng of Strep-Tactin Multimer activation reagent per mL of the composition.

In certain embodiments, a composition comprises a population of T cells, wherein the T cells are from a single healthy donor aged 18 to 35 years old and having a body mass index (BMI) less than 30 kg/m², and wherein the composition has one or more, two or more, three or more, four or more, five or more, six or more, seven or more, eight or more, nine or more, ten or more, eleven or more, twelve or more, or all the following attributes:
(i) at least about 80%, at least about 85%, or at least about 90% of the T cells are viable;
(ii) at least about 95%, at least about 96%, at least about 97%, at least about 98%, or at least about 99% of the T cells are CD2+CD5+;
(iii) at least about 50% of the T cells express CD19 CAR, such as the CD19 CAR having an amino acid sequence set forth in SEQ ID NO: 78 or SEQ ID NO: 138;
(iv) less than about 5% of the T cells are HLA-I+;
(v) the ratio of % TCR+ to % CAR+ T cells is less than about 0.008;
(vi) less than about 5% of total alleles have translocation between TRAC and B2M;
(vii) at least about 20,000,000 viable CD2+CD5+CD19 CAR+ cells per mL of the composition;
(viii) no detected bacterial growth;
(ix) no more than about 13.3 EU/mL endotoxin;
(x) no detected mycoplasma;
(xi) no cytokine-independent growth;
(xii) no significant unexpected karyotype; or
(xiii) negative for the presence of HIV, HTLV, HAV, HBV, HCV, CMV, EBV, HHV6, HHV7, HHV8, and B19.

In certain embodiments, a composition comprises a population of T cells, wherein the T cells are from a single healthy donor aged 18 to 35 years old and having a body mass index (BMI) less than 30 kg/m², and wherein the composition has one or more, two or more, three or more, four or more, five or more, six or more, seven or more, eight or more, nine or more, ten or more, eleven or more, twelve or more, or all the following attributes:
(i) at least about 80%, at least about 85%, or at least about 90% of the T cells are viable;
(ii) at least about 95%, at least about 96%, at least about 97%, at least about 98%, or at least about 99% of the T cells are CD2+CD5+;
(iii) at least about 50% of the T cells express CD19 CAR, such as the CD19 CAR having an amino acid sequence set forth in SEQ ID NO: 78 or SEQ ID NO: 138;
(iv) less than about 5% of the T cells are HLA-I+;
(v) the ratio of % TCR+ to % CAR+ T cells is less than about 0.008;
(vi) less than about 5% of total alleles have translocation between TRAC and B2M;
(vii) at least about 20,000,000 viable CD2+CD5+CD19 CAR+ cells per mL of the composition;
(viii) no detected bacterial growth;
(ix) no more than about 13.3 EU/mL endotoxin;
(x) no detected mycoplasma;
(xi) no cytokine-independent growth;
(xii) no significant unexpected karyotype; or
(xiii) negative for the presence of HIV, HTLV, HAV, HBV, HCV, CMV, EBV, HHV6, HHV7, HHV8, and B19, and wherein the composition has one or more of the following attributes:
(a) at least about 70% of total alleles, at least about 75% of total alleles, at least about 80% of total alleles, at least about 85% of total alleles, at least about 90% of total alleles, at least about 95% of total alleles, at least about 96% of total alleles, at least about 97% of total alleles, at least about 98% of total alleles, at least about 99% of total alleles, or 100% of total alleles have edited TRAC loci;
(b) less than about 0.1%, less than about 0.2%, less than about 0.3%, less than about 0.4%, less than about 0.5%, less than about 0.6%, less than about 0.7%, less than about 0.8%, less than about 0.9%, or less than about 1% of the T cells are TCR+ T cells;
(c) at least about 70% of total alleles, at least about 75% of total alleles, at least about 80% of total alleles, at least about 85% of total alleles, at least about 90% of total alleles, at least about 95% of total alleles, at least about 96% of total alleles, at least about 97% of total alleles, at least about 98% of total alleles, at least about 99% of total alleles, or 100% of total alleles have edited B2M loci;
(d) at least about 25% of total alleles, at least about 30% of total alleles, at least about 35% of total alleles, at least about 40% of total alleles, at least about 45% of total alleles, at least about 50% of total alleles, at least about 55% of total alleles, at least about 60% of total alleles, at least about 65% of total alleles, at least about 70% of total alleles, at least about 75% of total alleles, at least about 80% of total alleles, at least about 85% of total alleles, at least about 90% of total alleles, at least about 95% of total alleles, at least about 96% of total alleles, at least about 97% of total alleles, at least about 98% of total alleles, at least about 99% of total alleles, or 100% of total alleles have a nucleic acid encoding HLA-E(A) knocked-in at the B2M locus, such as a nucleic acid comprising the sequence set forth in SEQ ID NO: 86, or a nucleic acid encoding HLA-E(A) having an amino acid sequence set forth in SEQ ID NO: 81 or SEQ ID NO: 132;
(e) at least about 25% of total alleles, at least about 30% of total alleles, at least about 35% of total alleles, at least about 40% of total alleles, at least about 45% of total alleles, at least about 50% of total alleles, at least about 55% of total alleles, at least about 60% of total alleles, at least about 65% of total alleles, at least about 70% of total alleles, at least about 75% of total alleles, at least about 80% of total alleles, at least about 85% of total alleles, at least about 90% of total alleles, at least about 95% of total alleles, at least about 96% of total alleles, at least about 97% of total alleles, at least about 98% of total alleles, at least about 99% of total alleles, or 100% of total alleles have a nucleic acid encoding CD19 CAR knocked-in at the TRAC locus, such as a nucleic acid comprising the sequence set forth in SEQ ID NO: 136, or a nucleic acid encoding a CD19 CAR having an amino acid sequence set forth in SEQ ID NO: 78 or SEQ ID NO: 138;
(f) less than about 1 ng, less than about 2 ng, less than about 3 ng, less than about 4 ng, less than about 5 ng, less than about 6 ng, less than about 7 ng, less than about 8 ng, less than about 9 ng, less than about 10 ng, less than about 11 ng, less than about 12 ng, less than about 13 ng, less than about 14 ng, less than about 15 ng, less than about 16 ng, less than about 17 ng, less than about 18 ng, less than about 19 ng, or less than about 20 ng of Cas9 or Cas12a per mL of the composition;
(g) less than about 0.01 fg, less than about 0.02 fg, less than about 0.03 fg, less than about 0.04 fg, less than about 0.05 fg, less than about 0.06 fg, less than about 0.07 fg, less than about 0.08 fg, less than about 0.09 fg, or less than about 1.0 fg of Cas9 or Cas12a per T cell;
(h) less than about 1×10¹⁰, less than about 5×10¹⁰, less than about 1×10¹¹, less than about 5×10¹¹, or less than about 1×10¹² AAV6 capsids per mL of the composition;
(i) ten or fewer, nine or fewer, eight or fewer, seven or fewer, six or fewer, five or fewer, four or fewer, three or fewer, two or fewer, one or fewer, or no verified off-target indel sites at a genomic locus other than TRAC and B2M;
(j) less than about 0.5% of total alleles, less than about 1.0% of total alleles, less than about 1.5% of total alleles, less than about 2.0% of total alleles, less than about 2.5% of total alleles, less than about 3.0% of total alleles, less than about 3.5% of total alleles, or less than about 4.0% of total alleles have AAV6 integration at TRAC locus;
(k) less than about 0.5% of total alleles, less than about 0.6% of total alleles, less than about 0.7% of total alleles, less than about 0.8% of total alleles, less than about 0.9% of total alleles, less than about 1.0% of total alleles, less than about 1.1% of total alleles, less than about 1.2% of total alleles, less than about 1.3% of total alleles, less than about 1.4% of total alleles, less than about 1.5% of total alleles, less than about 1.6% of total alleles, less than about 1.7% of total alleles, less than about 1.8% of total alleles, less than about 1.9% of total alleles, or less than about 2.0% of total alleles have AAV6 integration at B2M locus;
(l) undetectable AAV6 integration at loci other than TRAC and B2M; or
(m) less than about 100 ng, less than about 150 ng, less than about 200 ng, less than about 250 ng, less than about 300 ng, less than about 350 ng, less than about 400 ng, less than about 450 ng, less than about 500 ng, less than about 550 ng, less than about 600 ng, less than about 650 ng, less than about 700 ng, less than about 750 ng, less than about 800 ng, less than about 850 ng, less than about 900 ng, less than about 950 ng, less than about 1000 ng, less than about 1100 ng, less than about 1200 ng, less than about 1300 ng, less than about 1400 ng, or less than about 1500 ng of Strep-Tactin Multimer activation reagent per mL of the composition.

In certain embodiments, a composition comprises a population of T cells, wherein the T cells are from a single healthy donor aged 18 to 35 years old and having a body mass index (BMI) less than 30 kg/m², and wherein the composition has one or more, two or more, three or more, four or more, five or more, six or more, seven or more, eight or more, nine or more, ten or more, eleven or more, twelve or more, or all the following attributes:
(i) at least about 70% of the T cells are viable;
(ii) at least about 90% of the T cells are CD2+CD5+;
(iii) at least about 50% of the T cells express CD19 CAR, such as the CD19 CAR having an amino acid sequence set forth in SEQ ID NO: 78 or SEQ ID NO: 138;
(iv) less than about 5% of the T cells are HLA-I+;
(v) the ratio of % TCR+ to % CAR+ T cells is less than about 0.008;
(vi) less than about 5% of total alleles have translocation between TRAC and B2M; or
(vii) at least about 20,000,000 viable CD2+CD5+CD19 CAR+ cells per mL of the composition;
(viii) no detected bacterial growth;
(ix) no more than about 13.3 EU/mL endotoxin;
(x) no detected mycoplasma;
(xi) no cytokine-independent growth;
(xii) no significant unexpected karyotype; or
(xiii) negative for the presence of HIV, HTLV, HAV, HBV, HCV, CMV, EBV, HHV6, HHV7, HHV8, and B19, and wherein the composition has one or more of the following attributes:
(a) at least about 90% of total alleles have edited TRAC loci;
(b) less than about 0.5% of the T cells are TCR+ T cells;
(c) at least about 90% of total alleles have edited B2M loci;
(d) at least about 50% of total alleles have a nucleic acid encoding HLA-E(A) knocked-in at the B2M locus, such as a nucleic acid comprising the sequence set forth in SEQ ID NO: 86, or a nucleic acid encoding HLA-E(A) having an amino acid sequence set forth in SEQ ID NO: 81 or SEQ ID NO: 132;
(e) at least about 50% of total alleles have a nucleic acid encoding CD19 CAR knocked-in at the TRAC locus, such as a nucleic acid comprising the sequence set forth in SEQ ID NO: 136, or a nucleic acid encoding a CD19 CAR having an amino acid sequence set forth in SEQ ID NO: 78 or SEQ ID NO: 138.
(f) less than about 2 ng of Cas9 or Cas12a per mL of the composition;
(g) less than about 0.05 fg of Cas9 or Cas12a per T cell;
(h) less than about 5×10¹¹ AAV6 capsids per mL of the composition;
(i) no verified off-target indel sites at a genomic locus other than TRAC and B2M;
(j) less than about 2.0% of total alleles have AAV6 integration at TRAC locus;
(k) less than about 1.0% of total alleles have AAV6 integration at B2M locus;
(l) undetectable AAV6 integration at loci other than TRAC and B2M; or
(m) less than about 900 ng of Strep-Tactin Multimer activation reagent per mL of the composition.

In certain embodiments, a composition comprises a population of T cells, wherein the T cells are from a single healthy donor aged 18 to 35 years old and having a body mass index (BMI) less than 30 kg/m², and wherein the composition has one or more, two or more, three or more, four or more, five or more, six or more, seven or more, eight or more, nine or more, ten or more, eleven or more, twelve or more, or all the following attributes:
(i) at least about 80%, at least about 85%, or at least about 90% of the T cells are viable;
(ii) at least about 95%, at least about 96%, at least about 97%, at least about 98%, or at least about 99% of the T cells are CD2+CD5+;
(iii) at least about 50% of the T cells express CD19 CAR, such as the CD19 CAR having an amino acid sequence set forth in SEQ ID NO: 78 or SEQ ID NO: 138;
(iv) less than about 5% of the T cells are HLA-I+;
(v) the ratio of % TCR+ to % CAR+ T cells is less than about 0.008;
(vi) less than about 5% of total alleles have translocation between TRAC and B2M;
(vii) at least about 20,000,000 viable CD2+CD5+CD19 CAR+ cells per mL of the composition;
(viii) no detected bacterial growth;
(ix) no more than about 13.3 EU/mL endotoxin;
(x) no detected mycoplasma;
(xi) no cytokine-independent growth;
(xii) no significant unexpected karyotype; or
(xiii) negative for the presence of HIV, HTLV, HAV, HBV, HCV, CMV, EBV, HHV6, HHV7, HHV8, and B19, and wherein the composition has one or more of the following attributes:
(a) at least about 90% of total alleles have edited TRAC loci;
(b) less than about 0.5% of the T cells are TCR+ T cells;
(c) at least about 90% of total alleles have edited B2M loci;
(d) at least about 50% of total alleles have a nucleic acid encoding HLA-E(A) knocked-in at the B2M locus, such as a nucleic acid comprising the sequence set forth in SEQ ID NO: 86, or a nucleic acid encoding HLA-E(A) having an amino acid sequence set forth in SEQ ID NO: 81 or SEQ ID NO: 132;
(e) at least about 50% of total alleles have a nucleic acid encoding CD19 CAR knocked-in at the TRAC locus, such as a nucleic acid comprising the sequence set forth in SEQ ID NO: 136, or a nucleic acid encoding a CD19 CAR having an amino acid sequence set forth in SEQ ID NO: 78 or SEQ ID NO: 138;
(f) less than about 2 ng of Cas9 or Cas12a per mL of the composition;
(g) less than about 0.05 fg of Cas9 or Cas12a per T cell;
(h) less than about 5×10¹¹ AAV6 capsids per mL of the composition;
(i) no verified off-target indel sites at a genomic locus other than TRAC and B2M;
(j) less than about 2.0% of total alleles have AAV6 integration at TRAC locus;
(k) less than about 1.0% of total alleles have AAV6 integration at B2M locus;
(l) undetectable AAV6 integration at loci other than TRAC and B2M; or
(m) less than about 900 ng of Strep-Tactin Multimer activation reagent per mL of the composition.

In any of the embodiments above relating to a composition comprising a population of T cells, the T cells can comprise (i) a modified TRAC locus comprising a transgene sequence comprising the sequence set forth in SEQ ID NO: 136, and (ii) a modified B2M locus comprising a transgene sequence comprising the sequence set forth in SEQ ID NO: 86.

In any of the embodiments above relating to a composition comprising a population of T cells, the T cells can comprise (i) a modified TRAC locus comprising a transgene sequence comprising the sequence set forth in SEQ ID NO: 94, and (ii) a modified B2M locus comprising a transgene sequence comprising the sequence set forth in SEQ ID NO: 137.

In certain embodiments, a composition comprises a population of T cells, wherein the composition has the following attributes:
(i) at least about 50%, at least about 55%, at least about 60%, at least about 65%, at least about 70%, at least about 75%, at least about 80%, at least about 85%, at least about 90%, or at least about 95% of the T cells are viable;
(ii) at least about 70%, at least about 75%, at least about 80%, at least about 85%, or at least about 90% of the T cells are CD2+CD5+;
(iii) at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the T cells express CD19 CAR, such as the CD19 CAR having an amino acid sequence set forth in SEQ ID NO: 78 or SEQ ID NO: 138;
(iv) less than about 2%, less than about 3%, less than about 4%, less than about 5%, less than about 6%, less than about 7%, less than about 8%, less than about 9%, or less than about 10% of the T cells are HLA-I+;
(v) the ratio of % TCR+ to % CAR+ T cells is less than about 0.005, less than about 0.006, less than about 0.007, less than about 0.008, less than about 0.009, or less than about 0.01;
(vi) less than about 1% of total alleles, less than about 2% of total alleles, less than about 3% of total alleles, less than about 4% of total alleles, less than about 5% of total alleles, less than about 6% of total alleles, less than about 7% of total alleles, less than about 8% of total alleles, less than about 9% of total alleles, or less than about 10% of total alleles have translocation between TRAC and B2M; and
(vii) at least about 5,000,000, at least about 10,000,000, at least about 15,000,000, at least about 20,000,000, at least about 25,000,000, at least about 30,000,000, at least about 35,000,000, at least about 40,000,000, at least about 45,000,000, or at least about 50,000,000 viable CD2+CD5+CD19 CAR+ cells per mL of the composition; and
wherein the T cells (i) are from a single healthy donor aged 18 to 35 years old and having a body mass index (BMI) less than 30 kg/m², and (ii) comprise (a) a modified TRAC locus comprising a transgene sequence comprising the sequence set forth in SEQ ID NO: 136, and (b) a modified B2M locus comprising a transgene sequence comprising the sequence set forth in SEQ ID NO: 86.

In certain embodiments, a composition comprises a population of T cells, wherein the composition has the following attributes:
(i) at least about 50%, at least about 55%, at least about 60%, at least about 65%, at least about 70%, at least about 75%, at least about 80%, at least about 85%, at least about 90%, or at least about 95% of the T cells are viable;
(ii) at least about 70%, at least about 75%, at least about 80%, at least about 85%, or at least about 90% of the T cells are CD2+CD5+;
(iii) at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the T cells express CD19 CAR, such as the CD19 CAR having an amino acid sequence set forth in SEQ ID NO: 78 or SEQ ID NO: 138;
(iv) less than about 2%, less than about 3%, less than about 4%, less than about 5%, less than about 6%, less than about 7%, less than about 8%, less than about 9%, or less than about 10% of the T cells are HLA-I+;
(v) the ratio of % TCR+ to % CAR+ T cells is less than about 0.005, less than about 0.006, less than about 0.007, less than about 0.008, less than about 0.009, or less than about 0.01;
(vi) less than about 1% of total alleles, less than about 2% of total alleles, less than about 3% of total alleles, less than about 4% of total alleles, less than about 5% of total alleles, less than about 6% of total alleles, less than about 7% of total alleles, less than about 8% of total alleles, less than about 9% of total alleles, or less than about 10% of total alleles have translocation between TRAC and B2M; and
(vii) at least about 5,000,000, at least about 10,000,000, at least about 15,000,000, at least about 20,000,000, at least about 25,000,000, at least about 30,000,000, at least about 35,000,000, at least about 40,000,000, at least about 45,000,000, or at least about 50,000,000 viable CD2+CD5+CD19 CAR+ cells per mL of the composition; wherein the T cells (i) are from a single healthy donor aged 18 to 35 years old and having a body mass index (BMI) less than 30 kg/m², and (ii) comprise (a) a modified TRAC locus comprising a transgene sequence comprising the sequence set forth in SEQ ID NO: 94, and (b) a modified B2M locus comprising a transgene sequence comprising the sequence set forth in SEQ ID NO: 137.

In certain embodiments, a composition comprises a population of T cells, wherein the composition has the following attributes:
(i) at least about 70% of the T cells are viable;
(ii) at least about 90% of the T cells are CD2+CD5+;
(iii) at least about 50% of the T cells express a CD19 CAR having an amino acid sequence set forth in SEQ ID NO: 78 or SEQ ID NO: 138;
(iv) less than about 5% of the T cells are HLA-I+;
(v) the ratio of % TCR+ to % CAR+ T cells is less than about 0.008;
(vi) less than about 5% of total alleles have translocation between TRAC and B2M; and
(vii) at least about 20,000,000 viable CD2+CD5+CD19 CAR+ cells per mL of the composition; wherein the T cells (i) are from a single healthy donor aged 18 to 35 years old and having a body mass index (BMI) less than 30 kg/m², and (ii) comprise (a) a modified TRAC locus comprising a transgene sequence comprising the sequence set forth in SEQ ID NO: 136, and (b) a modified B2M locus comprising a transgene sequence comprising the sequence set forth in SEQ ID NO: 86.

In certain embodiments, a composition comprises a population of T cells, wherein the composition has the following attributes:
(i) at least about 70% of the T cells are viable;
(ii) at least about 90% of the T cells are CD2+CD5+;
(iii) at least about 50% of the T cells express a CD19 CAR having an amino acid sequence set forth in SEQ ID NO: 78 or SEQ ID NO: 138;
(iv) less than about 5% of the T cells are HLA-I+;
(v) the ratio of % TCR+ to % CAR+ T cells is less than about 0.008;
(vi) less than about 5% translocation between TRAC and B2M; and
(vii) at least about 20,000,000 viable CD2+CD5+CD19 CAR+ cells per mL of the composition; wherein the T cells (i) are from a single healthy donor between aged 18 to 35 years old and having a body mass index (BMI) less than 30 kg/m², and (ii) comprise (a) a modified TRAC locus comprising a transgene sequence comprising the sequence set forth in SEQ ID NO: 94, and (b) a modified B2M locus comprising a transgene sequence comprising the sequence set forth in SEQ ID NO: 137.

In certain embodiments, a composition comprises a population of T cells, wherein the composition has the following attributes:
(i) at least about 80%, at least about 85%, or at least about 90% of the T cells are viable;
(ii) at least about 95%, at least about 96%, at least about 97%, at least about 98%, or at least about 99% of the T cells are CD2+CD5+;
(iii) at least about 50% of the T cells express CD19 CAR, such as the CD19 CAR having an amino acid sequence set forth in SEQ ID NO: 78 or SEQ ID NO: 138;
(iv) less than about 5% of the T cells are HLA-I+;
(v) the ratio of % TCR+ to % CAR+ T cells is less than about 0.008;
(vi) less than about 5% of total alleles have translocation between TRAC and B2M; and
(vii) at least about 20,000,000 viable CD2+CD5+CD19 CAR+ cells per mL of the composition; wherein the T cells (i) are from a single healthy donor aged 18 to 35 years old and having a body mass index (BMI) less than 30 kg/m², and (ii) comprise (a) a modified TRAC locus comprising a transgene sequence comprising the sequence set forth in SEQ ID NO: 94, and (b) a modified B2M locus comprising a transgene sequence comprising the sequence set forth in SEQ ID NO: 137.

In certain embodiments, a composition comprises a population of T cells, wherein the composition has the following attributes:
(i) at least about 80%, at least about 85%, or at least about 90% of the T cells are viable;
(ii) at least about 95%, at least about 96%, at least about 97%, at least about 98%, or at least about 99% of the T cells are CD2+CD5+;
(iii) at least about 50% of the T cells express CD19 CAR, such as the CD19 CAR having an amino acid sequence set forth in SEQ ID NO: 78 or SEQ ID NO: 138;
(iv) less than about 5% of the T cells are HLA-I+;
(v) the ratio of % TCR+ to % CAR+ T cells is less than about 0.008;
(vi) less than about 5% of total alleles have translocation between TRAC and B2M; and
(vii) at least about 20,000,000 viable CD2+CD5+CD19 CAR+ cells per mL of the composition; wherein the T cells (i) are from a single healthy donor aged 18 to 35 years old and having a body mass index (BMI) less than 30 kg/m², and (ii) comprise (a) a modified TRAC locus comprising a transgene sequence comprising the sequence set forth in SEQ ID NO: 94, and (b) a modified B2M locus comprising a transgene sequence comprising the sequence set forth in SEQ ID NO: 137; and wherein the composition has one or more of the following attributes:
(a) at least about 90% of total alleles have edited TRAC loci;
(b) less than about 0.5% of the T cells are TCR+ T cells;
(c) at least about 90% of total alleles have edited B2M loci;
(d) at least about 50% of total alleles have a nucleic acid encoding HLA-E(A) knocked-in at the B2M locus, such as a nucleic acid comprising the sequence set forth in SEQ ID NO: 86, or a nucleic acid encoding HLA-E(A) having an amino acid sequence set forth in SEQ ID NO: 81 or SEQ ID NO: 132;
(e) at least about 50% of total alleles have a nucleic acid encoding CD19 CAR knocked-in at the TRAC locus, such as a nucleic acid comprising the sequence set forth in SEQ ID NO: 136, or a nucleic acid encoding a CD19 CAR having an amino acid sequence set forth in SEQ ID NO: 78 or SEQ ID NO: 138;
(f) less than about 2 ng of Cas9 or Cas12a per mL of the composition;
(g) less than about 0.05 fg of Cas9 or Cas12a per T cell;
(h) less than about 5×10¹¹ AAV6 capsids per mL of the composition;
(i) no verified off-target indel sites at a genomic locus other than TRAC and B2M;
(j) less than about 2.0% of total alleles have AAV6 integration at TRAC locus;
(k) less than about 1.0% of total alleles have AAV6 integration at B2M locus;
(l) undetectable AAV6 integration at loci other than TRAC and B2M; or
(m) less than about 900 ng of Strep-Tactin Multimer activation reagent per mL of the composition.

In certain embodiments, a composition comprises a population of T cells, wherein the composition has one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, twenty-four or more, twenty-five or more, or all the following attributes:
(i) at least about 80%, at least about 85%, or at least about 90% of the T cells are viable;
(ii) at least about 95%, at least about 96%, at least about 97%, at least about 98%, or at least about 99% of the T cells are CD2+CD5+;
(iii) at least about 50% of the T cells express CD19 CAR, such as the CD19 CAR having an amino acid sequence set forth in SEQ ID NO: 78 or SEQ ID NO: 138;
(iv) less than about 5% of the T cells are HLA-I+;
(v) the ratio of % TCR+ to % CAR+ T cells is less than about 0.008;
(vi) less than about 5% of total alleles have translocation between TRAC and B2M;
(vii) at least about 20,000,000 viable CD2+CD5+CD19 CAR+ cells per mL of the composition;
(viii) no detected bacterial growth;
(ix) no more than about 13.3 EU/mL endotoxin;
(x) no detected mycoplasma;
(xi) no cytokine-independent growth;
(xii) no significant unexpected karyotype;
(xiii) negative for the presence of HIV, HTLV, HAV, HBV, HCV, CMV, EBV, HHV6, HHV7, HHV8, and B19;
(xiv) at least about 90% of total alleles have edited TRAC loci;
(xv) less than about 0.5% of the T cells are TCR+ T cells;
(xvi) at least about 90% of total alleles have edited B2M loci;
(xvii) at least about 50% of total alleles have a nucleic acid encoding HLA-E(A) knocked-in at the B2M locus, such as a nucleic acid comprising the sequence set forth in SEQ ID NO: 86, or a nucleic acid encoding HLA-E(A) having an amino acid sequence set forth in SEQ ID NO: 81 or SEQ ID NO: 132;
(xviii) at least about 50% of total alleles have a nucleic acid encoding CD19 CAR knocked-in at the TRAC locus, such as a nucleic acid comprising the sequence set forth in SEQ ID NO: 136, or a nucleic acid encoding a CD19 CAR having an amino acid sequence set forth in SEQ ID NO: 78 or SEQ ID NO: 138;
(xix) less than about 2 ng of Cas9 or Cas12a per mL of the composition;
(xx) less than about 0.05 fg of Cas9 or Cas12a per T cell;
(xxi) less than about 5×10¹¹ AAV6 capsids per mL of the composition;
(xxii) no verified off-target indel sites at a genomic locus other than TRAC and B2M;
(xxiii) less than about 2.0% of total alleles have AAV6 integration at TRAC locus;
(xxiv) less than about 1.0% of total alleles have AAV6 integration at B2M locus;
(xxv) undetectable AAV6 integration at loci other than TRAC and B2M; or
(xxvi) less than about 900 ng of Strep-Tactin Multimer activation reagent per mL of the composition; wherein the T cells (i) are from a single healthy donor aged 18 to 35 years old and having a body mass index (BMI) less than 30 kg/m², and (ii) comprise (a) a modified TRAC locus comprising a transgene sequence comprising the sequence set forth in SEQ ID NO: 94, and (b) a modified B2M locus comprising a transgene sequence comprising the sequence set forth in SEQ ID NO: 137.

In any of the embodiments above relating to a composition comprising a population of T cells, the composition can be negative for HIV-1, HIV-2, HTLV-1, HTLV-2, HAV, HBV, HCV, CMV, EBV, HHV6, HHV7, HHV8 and B19 viruses.

In certain embodiments, a composition comprises a population of T cells, wherein the composition has one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, or all the following attributes:
(i) at least about 70% of the T cells are viable;
(ii) at least about 10% of total alleles have edited TRAC loci;
(iii) at least about 10% of total alleles have edited B2M loci;
(iv) at least about 10% of total alleles have a nucleic acid encoding HLA-E(A) knocked-in at the B2M locus, such as a nucleic acid comprising the sequence set forth in SEQ ID NO: 86, or a nucleic acid encoding HLA-E(A) having an amino acid sequence set forth in SEQ ID NO: 81 or SEQ ID NO: 132;
(v) at least about 10% of total alleles have a nucleic acid encoding CD19 CAR knocked-in at the TRAC locus, such as a nucleic acid comprising the sequence set forth in SEQ ID NO: 136, or a nucleic acid encoding a CD19 CAR having an amino acid sequence set forth in SEQ ID NO: 78 or SEQ ID NO: 138;
(vi) at least about 90% of the T cells are CD2+CD5+;
(vii) at least about 50% of the T cells are CD2+CD5+ and express CD19 CAR, such as the CD19 CAR having an amino acid sequence set forth in SEQ ID NO: 78 or SEQ ID NO: 138;
(viii) at least about 7,500,000 viable CD2+CD5+CD19 CAR+ cells per mL of the composition;
(ix) no detected bacterial growth;
(x) less than about 5 EU/mL endotoxin;
(xi) no detected mycoplasma;
(xii) less than about 70,000 TCR+ cells/kg;
(xiii) no cytokine-independent growth;
(xiv) no significant unexpected karyotype (xv) less than about 5% of total alleles have translocation between TRAC and B2M;
(xvi) negative for the presence of HIV-1, HIV-2, HTLV-1, HTLV-2, HAV, HBV, HCV, CMV, EBV, HHV6, HHV7, HHV8, and B19;
(xvii) the T cells are from a healthy donor aged 18 to 35 years old; or
(xviii) the T cells are from a single healthy donor having a body mass index (BMI) less than 30 kg/m².

In certain embodiments, a composition comprises a population of T cells, wherein the T cells are from a healthy donor aged 18 to 35 years old and having a body mass index (BMI) less than 30 kg/m², and wherein the composition has one or more, two or more, three or more, four or more, five or more, six or more, seven or more, eight or more, nine or more, ten or more, eleven or more, twelve or more, thirteen or more, fourteen or more, fifteen or more, or all the following attributes:
(i) at least about 70% of the T cells are viable;
(ii) at least about 10% of total alleles have edited TRAC loci;
(iii) at least about 10% of total alleles have edited B2M loci;
(iv) at least about 10% of total alleles have a nucleic acid encoding HLA-E(A) knocked-in at the B2M locus, such as a nucleic acid comprising the sequence set forth in SEQ ID NO: 86, or a nucleic acid encoding HLA-E(A) having an amino acid sequence set forth in SEQ ID NO: 81 or SEQ ID NO: 132;
(v) at least about 10% of total alleles have a nucleic acid encoding CD19 CAR knocked-in at the TRAC locus, such as a nucleic acid comprising the sequence set forth in SEQ ID NO: 136, or a nucleic acid encoding a CD19 CAR having an amino acid sequence set forth in SEQ ID NO: 78 or SEQ ID NO: 138;
(vi) at least about 90% of the T cells are CD2+CD5+;
(vii) at least about 50% of the T cells are CD2+CD5+ and express CD19 CAR, such as the CD19 CAR having an amino acid sequence set forth in SEQ ID NO: 78 or SEQ ID NO: 138;
(viii) at least about 7,500,000 viable CD2+CD5+CD19 CAR+ cells per mL of the composition;
(ix) no detected bacterial growth;
(x) less than about 5 EU/mL endotoxin;
(xi) no detected mycoplasma;
(xii) less than about 70,000 TCR+ cells/kg;
(xiii) no cytokine-independent growth;
(xiv) no significant unexpected karyotype
(xv) less than about 5% of total alleles have translocation between TRAC and B2M; or
(xvi) negative for the presence of HIV-1, HIV-2, HTLV-1, HTLV-2, HAV, HBV, HCV, CMV, EBV, HHV6, HHV7, HHV8, and B19.

In certain embodiments, a composition comprises a population of T cells, wherein the T cells are from a healthy donor aged 18 to 35 years old and having a body mass index (BMI) less than 30 kg/m², and wherein the composition has one or more, two or more, three or more, four or more, five or more, six or more, seven or more, eight or more, nine or more, ten or more, eleven or more, twelve or more, thirteen or more, fourteen or more, fifteen or more, or all the following attributes:
(i) at least about 70% of the T cells are viable;
(ii) at least about 10% of total alleles have edited TRAC loci;
(iii) at least about 10% of total alleles have edited B2M loci;
(iv) at least about 10% of total alleles have a nucleic acid encoding HLA-E(A) knocked-in at the B2M locus, such as a nucleic acid comprising the sequence set forth in SEQ ID NO: 86, or a nucleic acid encoding HLA-E(A) having an amino acid sequence set forth in SEQ ID NO: 81 or SEQ ID NO: 132;
(v) at least about 10% of total alleles have a nucleic acid encoding CD19 CAR knocked-in at the TRAC locus, such as a nucleic acid comprising the sequence set forth in SEQ ID NO: 136, or a nucleic acid encoding a CD19 CAR having an amino acid sequence set forth in SEQ ID NO: 78 or SEQ ID NO: 138;
(vi) at least about 90% of the T cells are CD2+CD5+;
(vii) at least about 50% of the T cells are CD2+CD5+ and express CD19 CAR, such as the CD19 CAR having an amino acid sequence set forth in SEQ ID NO: 78 or SEQ ID NO: 138;
(viii) at least about 7,500,000 viable CD2+CD5+CD19 CAR+ cells per mL of the composition;
(ix) no detected bacterial growth;
(x) less than about 5 EU/mL endotoxin;
(xi) no detected mycoplasma;
(xii) less than about 70,000 TCR+ cells/kg;
(xiii) no cytokine-independent growth;
(xiv) no significant unexpected karyotype
(xv) less than about 5% of total alleles have translocation between TRAC and B2M; or
(xvi) negative for the presence of HIV-1, HIV-2, HTLV-1, HTLV-2, HAV, HBV, HCV, CMV, EBV, HHV6, HHV7, HHV8, and B19, and wherein the composition has one or more of the following attributes:
(a) less than about 0.1%, less than about 0.2%, less than about 0.3%, less than about 0.4%, less than about 0.5%, less than about 0.6%, less than about 0.7%, less than about 0.8%, less than about 0.9%, or less than about 1% of the T cells are TCR+ T cells;
(b) less than about 1 ng, less than about 2 ng, less than about 3 ng, less than about 4 ng, less than about 5 ng, less than about 6 ng, less than about 7 ng, less than about 8 ng, less than about 9 ng, less than about 10 ng, less than about 11 ng, less than about 12 ng, less than about 13 ng, less than about 14 ng, less than about 15 ng, less than about 16 ng, less than about 17 ng, less than about 18 ng, less than about 19 ng, or less than about 20 ng of Cas9 or Cas12a per mL of the composition;
(c) less than about 0.01 fg, less than about 0.02 fg, less than about 0.03 fg, less than about 0.04 fg, less than about 0.05 fg, less than about 0.06 fg, less than about 0.07 fg, less than about 0.08 fg, less than about 0.09 fg, or less than about 1.0 fg of Cas9 or Cas12a per T cell;
(d) less than about 1×10¹⁰, less than about 5×10¹⁰, less than about 1×10¹¹, less than about 5×10¹¹, or less than about 1×10¹² AAV6 capsids per mL of the composition;
(e) ten or fewer, nine or fewer, eight or fewer, seven or fewer, six or fewer, five or fewer, four or fewer, three or fewer, two or fewer, one or fewer, or no verified off-target indel sites at a genomic locus other than TRAC and B2M;
(f) less than about 0.5% of total alleles, less than about 1.0% of total alleles, less than about 1.5% of total alleles, less than about 2.0% of total alleles, less than about 2.5% of total alleles, less than about 3.0% of total alleles, less than about 3.5% of total alleles, or less than about 4.0% of total alleles have AAV6 integration at TRAC locus;
(g) less than about 0.5% of total alleles, less than about 0.6% of total alleles, less than about 0.7% of total alleles, less than about 0.8% of total alleles, less than about 0.9% of total alleles, less than about 1.0% of total alleles, less than about 1.1% of total alleles, less than about 1.2% of total alleles, less than about 1.3% of total alleles, less than about 1.4% of total alleles, less than about 1.5% of total alleles, less than about 1.6% of total alleles, less than about 1.7% of total alleles, less than about 1.8% of total alleles, less than about 1.9% of total alleles, or less than about 2.0% of total alleles have AAV6 integration at B2M locus;
(h) undetectable AAV6 integration at loci other than TRAC and B2M; or
(i) less than about 100 ng, less than about 150 ng, less than about 200 ng, less than about 250 ng, less than about 300 ng, less than about 350 ng, less than about 400 ng, less than about 450 ng, less than about 500 ng, less than about 550 ng, less than about 600 ng, less than about 650 ng, less than about 700 ng, less than about 750 ng, less than about 800 ng, less than about 850 ng, less than about 900 ng, less than about 950 ng, less than about 1000 ng, less than about 1100 ng, less than about 1200 ng, less than about 1300 ng, less than about 1400 ng, or less than about 1500 ng of Strep-Tactin Multimer activation reagent per mL of the composition.

In certain embodiments, a composition comprises a population of T cells, wherein the T cells are from a healthy donor aged 18 to 35 years old and having a body mass index (BMI) less than 30 kg/m², and wherein the composition has one or more, two or more, three or more, four or more, five or more, six or more, seven or more, eight or more, nine or more, ten or more, eleven or more, twelve or more, thirteen or more, fourteen or more, fifteen or more, or all the following attributes:
(i) at least about 70% of the T cells are viable;
(ii) at least about 10% of total alleles have edited TRAC loci;
(iii) at least about 10% of total alleles have edited B2M loci;
(iv) at least about 10% of total alleles have a nucleic acid encoding HLA-E(A) knocked-in at the B2M locus, such as a nucleic acid comprising the sequence set forth in SEQ ID NO: 86, or a nucleic acid encoding HLA-E(A) having an amino acid sequence set forth in SEQ ID NO: 81 or SEQ ID NO: 132;
(v) at least about 10% of total alleles have a nucleic acid encoding CD19 CAR knocked-in at the TRAC locus, such as a nucleic acid comprising the sequence set forth in SEQ ID NO: 136, or a nucleic acid encoding a CD19 CAR having an amino acid sequence set forth in SEQ ID NO: 78 or SEQ ID NO: 138;
(vi) at least about 90% of the T cells are CD2+CD5+;
(vii) at least about 50% of the T cells are CD2+CD5+ and express CD19 CAR, such as the CD19 CAR having an amino acid sequence set forth in SEQ ID NO: 78 or SEQ ID NO: 138;
(viii) at least about 7,500,000 viable CD2+CD5+CD19 CAR+ cells per mL of the composition;
(ix) no detected bacterial growth;
(x) less than about 5 EU/mL endotoxin;
(xi) no detected mycoplasma;
(xii) less than about 70,000 TCR+ cells/kg;
(xiii) no cytokine-independent growth;
(xiv) no significant unexpected karyotype
(xv) less than about 5% of total alleles have translocation between TRAC and B2M; or
(xvi) negative for the presence of HIV-1, HIV-2, HTLV-1, HTLV-2, HAV, HBV, HCV, CMV, EBV, HHV6, HHV7, HHV8, and B19, and wherein the composition has one or more of the following attributes:
(a) less than about 0.5% of the T cells are TCR+ T cells;
(b) less than about 2 ng of Cas9 or Cas12a per mL of the composition;
(c) less than about 0.05 fg of Cas9 or Cas12a per T cell;
(d) less than about 5×10¹¹ AAV6 capsids per mL of the composition;
(e) no verified off-target indel sites at a genomic locus other than TRAC and B2M;
(f) less than about 2.0% of total alleles have AAV6 integration at TRAC locus;
(g) less than about 1.0% of total alleles have AAV6 integration at B2M locus;
(h) undetectable AAV6 integration at loci other than TRAC and B2M; or
(i) less than about 900 ng of Strep-Tactin Multimer activation reagent per mL of the composition.

In certain embodiments, a composition comprises a population of T cells, wherein the composition has one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, or all the following attributes:
(i) at least about 70% of the T cells are viable determined by fluorescent microscopy;
(ii) at least about 10% of total alleles have edited TRAC loci determined by ddPCR;
(iii) at least about 10% of total alleles have edited B2M loci determined by ddPCR;
(iv) at least about 10% of total alleles have a nucleic acid encoding HLA-E(A) knocked-in at the B2M locus, such as a nucleic acid comprising the sequence set forth in SEQ ID NO: 86, or a nucleic acid encoding HLA-E(A) having an amino acid sequence set forth in SEQ ID NO: 81 or SEQ ID NO: 132, determined by ddPCR;
(v) at least about 10% of total alleles have a nucleic acid encoding CD19 CAR knocked-in at the TRAC locus, such as a nucleic acid comprising the sequence set forth in SEQ ID NO: 136, or a nucleic acid encoding a CD19 CAR having an amino acid sequence set forth in SEQ ID NO: 78 or SEQ ID NO: 138, determined by ddPCR;
(vi) at least about 90% of the T cells are CD2+CD5+ determined by flow cytometry;
(vii) at least about 50% of the T cells are CD2+CD5+ and express CD19 CAR, such as the CD19 CAR having an amino acid sequence set forth in SEQ ID NO: 78 or SEQ ID NO: 138, determined by flow cytometry;
(viii) at least about 7,500,000 viable CD2+CD5+CD19 CAR+ cells per mL of the composition determined by flow cytometry;
(ix) no detected bacterial growth determined by BacT/ALERT 3D;
(x) less than about 5 EU/mL endotoxin determined by Limulus Amoebocyte Lysate (LAL);
(xi) no detected mycoplasma determined by qPCR;
(xii) less than about 70,000 TCR+ cells/kg determined by flow cytometry and calculated by multiplying the % TCR+ cells by viable cell number to get % TCR cells/mL, then multiplying the % TCR cells/mL by volume to obtain total number of TCR+ cells, then dividing the total number TCR+ cells by 60 kg patient weight;
(xiii) no cytokine-independent growth between day 31 and day 70 in a cell-based assay having a limit of detection (LOD) of about 1.5E5 cells/mL;
(xiv) no significant unexpected karyotype determined by microscopy, wherein a significant unexpected karyotype is the same specific aberration or aberrant ploidy in more than 6 cells and occurs in two out of three test replicates or three out of three test replicates;
(xv) less than about 5% of total alleles have translocation between TRAC and B2M determined by ddPCR;
(xvi) negative for the presence of HIV-1, HIV-2, HTLV-1, HTLV-2, HAV, HBV, HCV, CMV, EBV, HHV6, HHV7, HHV8, and B19 determined by PCR;
(xvii) the T cells are from a healthy donor aged 18 to 35 years old; or
(xviii) the T cells are from a single healthy donor having a body mass index (BMI) less than 30 kg/m².

In certain embodiments, a composition comprises a population of T cells, wherein the T cells are from a healthy donor aged 18 to 35 years old and having a body mass index (BMI) less than 30 kg/m², and wherein the composition has one or more, two or more, three or more, four or more, five or more, six or more, seven or more, eight or more, nine or more, ten or more, eleven or more, twelve or more, thirteen or more, fourteen or more, fifteen or more, or all the following attributes:
(i) at least about 70% of the T cells are viable determined by fluorescent microscopy;
(ii) at least about 10% of total alleles have edited TRAC loci determined by ddPCR;
(iii) at least about 10% of total alleles have edited B2M loci determined by ddPCR;
(iv) at least about 10% of total alleles have a nucleic acid encoding HLA-E(A) knocked-in at the B2M locus, such as a nucleic acid comprising the sequence set forth in SEQ ID NO: 86, or a nucleic acid encoding HLA-E(A) having an amino acid sequence set forth in SEQ ID NO: 81 or SEQ ID NO: 132, determined by ddPCR;
(v) at least about 10% of total alleles have a nucleic acid encoding CD19 CAR knocked-in at the TRAC locus, such as a nucleic acid comprising the sequence set forth in SEQ ID NO: 136, or a nucleic acid encoding a CD19 CAR having an amino acid sequence set forth in SEQ ID NO: 78 or SEQ ID NO: 138, determined by ddPCR;
(vi) at least about 90% of the T cells are CD2+CD5+ determined by flow cytometry;
(vii) at least about 50% of the T cells are CD2+CD5+ and express CD19 CAR, such as the CD19 CAR having an amino acid sequence set forth in SEQ ID NO: 78 or SEQ ID NO: 138, determined by flow cytometry;
(viii) at least about 7,500,000 viable CD2+CD5+CD19 CAR+ cells per mL of the composition determined by flow cytometry;
(ix) no detected bacterial growth determined by BacT/ALERT 3D;
(x) less than about 5 EU/mL endotoxin determined by Limulus Amoebocyte Lysate (LAL);
(xi) no detected mycoplasma determined by qPCR;
(xii) less than about 70,000 TCR+ cells/kg determined by flow cytometry and calculated by multiplying the % TCR+ cells by viable cell number to get % TCR cells/mL, then multiplying the % TCR cells/mL by volume to obtain total number of TCR+ cells, then dividing the total number TCR+ cells by 60 kg patient weight;
(xiii) no cytokine-independent growth between day 31 and day 70 in a cell-based assay having a limit of detection (LOD) of about 1.5E5 cells/mL;
(xiv) no significant unexpected karyotype determined by microscopy, wherein a significant unexpected karyotype is the same specific aberration or aberrant ploidy in more than 6 cells and occurs in two out of three test replicates or three out of three test replicates;
(xv) less than about 5% of total alleles have translocation between TRAC and B2M determined by ddPCR; or
(xvi) negative for the presence of HIV-1, HIV-2, HTLV-1, HTLV-2, HAV, HBV, HCV, CMV, EBV, HHV6, HHV7, HHV8, and B19 determined by PCR.

In certain embodiments, a composition comprises a population of T cells, wherein the T cells are from a healthy donor aged 18 to 35 years old and having a body mass index (BMI) less than 30 kg/m², and wherein the composition has one or more, two or more, three or more, four or more, five or more, six or more, seven or more, eight or more, nine or more, ten or more, eleven or more, twelve or more, thirteen or more, fourteen or more, fifteen or more, or all the following attributes:
(i) at least about 70% of the T cells are viable determined by fluorescent microscopy;
(ii) at least about 10% of total alleles have edited TRAC loci determined by ddPCR;
(iii) at least about 10% of total alleles have edited B2M loci determined by ddPCR;
(iv) at least about 10% of total alleles have a nucleic acid encoding HLA-E(A) knocked-in at the B2M locus, such as a nucleic acid comprising the sequence set forth in SEQ ID NO: 86, or a nucleic acid encoding HLA-E(A) having an amino acid sequence set forth in SEQ ID NO: 81 or SEQ ID NO: 132, determined by ddPCR;
(v) at least about 10% of total alleles have a nucleic acid encoding CD19 CAR knocked-in at the TRAC locus, such as a nucleic acid comprising the sequence set forth in SEQ ID NO: 136, or a nucleic acid encoding a CD19 CAR having an amino acid sequence set forth in SEQ ID NO: 78 or SEQ ID NO: 138, determined by ddPCR;
(vi) at least about 90% of the T cells are CD2+CD5+ determined by flow cytometry;
(vii) at least about 50% of the T cells are CD2+CD5+ and express CD19 CAR, such as the CD19 CAR having an amino acid sequence set forth in SEQ ID NO: 78 or SEQ ID NO: 138, determined by flow cytometry;
(viii) at least about 7,500,000 viable CD2+CD5+CD19 CAR+ cells per mL of the composition determined by flow cytometry;
(ix) no detected bacterial growth determined by BacT/ALERT 3D;
(x) less than about 5 EU/mL endotoxin determined by Limulus Amoebocyte Lysate (LAL);
(xi) no detected mycoplasma determined by qPCR;
(xii) less than about 70,000 TCR+ cells/kg determined by flow cytometry and calculated by multiplying the % TCR+ cells by viable cell number to get % TCR cells/mL, then multiplying the % TCR cells/mL by volume to obtain total number of TCR+ cells, then dividing the total number TCR+ cells by 60 kg patient weight;
(xiii) no cytokine-independent growth between day 31 and day 70 in a cell-based assay having a limit of detection (LOD) of about 1.5E5 cells/mL;
(xiv) no significant unexpected karyotype determined by microscopy, wherein a significant unexpected karyotype is the same specific aberration or aberrant ploidy in more than 6 cells and occurs in two out of three test replicates or three out of three test replicates;
(xv) less than about 5% of total alleles have translocation between TRAC and B2M determined by ddPCR; or
(xvi) negative for the presence of HIV-1, HIV-2, HTLV-1, HTLV-2, HAV, HBV, HCV, CMV, EBV, HHV6, HHV7, HHV8, and B19 determined by PCR, and wherein the composition has one or more of the following attributes:
(a) less than about 0.1%, less than about 0.2%, less than about 0.3%, less than about 0.4%, less than about 0.5%, less than about 0.6%, less than about 0.7%, less than about 0.8%, less than about 0.9%, or less than about 1% of the T cells are TCR+ T cells;
(b) less than about 1 ng, less than about 2 ng, less than about 3 ng, less than about 4 ng, less than about 5 ng, less than about 6 ng, less than about 7 ng, less than about 8 ng, less than about 9 ng, less than about 10 ng, less than about 11 ng, less than about 12 ng, less than about 13 ng, less than about 14 ng, less than about 15 ng, less than about 16 ng, less than about 17 ng, less than about 18 ng, less than about 19 ng, or less than about 20 ng of Cas9 or Cas12a per mL of the composition;
(c) less than about 0.01 fg, less than about 0.02 fg, less than about 0.03 fg, less than about 0.04 fg, less than about 0.05 fg, less than about 0.06 fg, less than about 0.07 fg, less than about 0.08 fg, less than about 0.09 fg, or less than about 1.0 fg of Cas9 or Cas12a per T cell;
(d) less than about 1×10¹⁰, less than about 5×10¹⁰, less than about 1×10¹¹, less than about 5×10¹¹, or less than about 1×10¹² AAV6 capsids per mL of the composition;
(e) ten or fewer, nine or fewer, eight or fewer, seven or fewer, six or fewer, five or fewer, four or fewer, three or fewer, two or fewer, one or fewer, or no verified off-target indel sites at a genomic locus other than TRAC and B2M;
(f) less than about 0.5% of total alleles, less than about 1.0% of total alleles, less than about 1.5% of total alleles, less than about 2.0% of total alleles, less than about 2.5% of total alleles, less than about 3.0% of total alleles, less than about 3.5% of total alleles, or less than about 4.0% of total alleles have AAV6 integration at TRAC locus;
(g) less than about 0.5% of total alleles, less than about 0.6% of total alleles, less than about 0.7% of total alleles, less than about 0.8% of total alleles, less than about 0.9% of total alleles, less than about 1.0% of total alleles, less than about 1.1% of total alleles, less than about 1.2% of total alleles, less than about 1.3% of total alleles, less than about 1.4% of total alleles, less than about 1.5% of total alleles, less than about 1.6% of total alleles, less than about 1.7% of total alleles, less than about 1.8% of total alleles, less than about 1.9% of total alleles, or less than about 2.0% of total alleles have AAV6 integration at B2M locus;
(h) undetectable AAV6 integration at loci other than TRAC and B2M; or
(i) less than about 100 ng, less than about 150 ng, less than about 200 ng, less than about 250 ng, less than about 300 ng, less than about 350 ng, less than about 400 ng, less than about 450 ng, less than about 500 ng, less than about 550 ng, less than about 600 ng, less than about 650 ng, less than about 700 ng, less than about 750 ng, less than about 800 ng, less than about 850 ng, less than about 900 ng, less than about 950 ng, less than about 1000 ng, less than about 1100 ng, less than about 1200 ng, less than about 1300 ng, less than about 1400 ng, or less than about 1500 ng of Strep-Tactin Multimer activation reagent per mL of the composition.

In certain embodiments, a composition comprises a population of T cells, wherein the T cells are from a healthy donor aged 18 to 35 years old and having a body mass index (BMI) less than 30 kg/m², and wherein the composition has one or more, two or more, three or more, four or more, five or more, six or more, seven or more, eight or more, nine or more, ten or more, eleven or more, twelve or more, thirteen or more, fourteen or more, fifteen or more, or all the following attributes:

(i) at least about 70% of the T cells are viable determined by fluorescent microscopy;
(ii) at least about 10% of total alleles have edited TRAC loci determined by ddPCR;
(iii) at least about 10% of total alleles have edited B2M loci determined by ddPCR;
(iv) at least about 10% of total alleles have a nucleic acid encoding HLA-E(A) knocked-in at the B2M locus, such as a nucleic acid comprising the sequence set forth in SEQ ID NO: 86, or a nucleic acid encoding HLA-E(A) having an amino acid sequence set forth in SEQ ID NO: 81 or SEQ ID NO: 132, determined by ddPCR;
(v) at least about 10% of total alleles have a nucleic acid encoding CD19 CAR knocked-in at the TRAC locus, such as a nucleic acid comprising the sequence set forth in SEQ ID NO: 136, or a nucleic acid encoding a CD19 CAR having an amino acid sequence set forth in SEQ ID NO: 78 or SEQ ID NO: 138, determined by ddPCR;
(vi) at least about 90% of the T cells are CD2+CD5+ determined by flow cytometry;
(vii) at least about 50% of the T cells are CD2+CD5+ and express CD19 CAR, such as the CD19 CAR having an amino acid sequence set forth in SEQ ID NO: 78 or SEQ ID NO: 138, determined by flow cytometry;
(viii) at least about 7,500,000 viable CD2+CD5+CD19 CAR+ cells per mL of the composition determined by flow cytometry;
(ix) no detected bacterial growth determined by BacT/ALERT 3D;
(x) less than about 5 EU/mL endotoxin determined by Limulus Amoebocyte Lysate (LAL);
(xi) no detected mycoplasma determined by qPCR;
(xii) less than about 70,000 TCR+ cells/kg determined by flow cytometry and calculated by multiplying the % TCR+ cells by viable cell number to get % TCR cells/mL, then multiplying the % TCR cells/mL by volume to obtain total number of TCR+ cells, then dividing the total number TCR+ cells by 60 kg patient weight;
(xiii) no cytokine-independent growth between day 31 and day 70 in a cell-based assay having a limit of detection (LOD) of about 1.5E5 cells/mL;
(xiv) no significant unexpected karyotype determined by microscopy, wherein a significant unexpected karyotype is the same specific aberration or aberrant ploidy in more than 6 cells and occurs in two out of three test replicates or three out of three test replicates;
(xv) less than about 5% of total alleles have translocation between TRAC and B2M determined by ddPCR; or
(xvi) negative for the presence of HIV-1, HIV-2, HTLV-1, HTLV-2, HAV, HBV, HCV, CMV, EBV, HHV6, HHV7, HHV8, and B19 determined by PCR, and wherein the composition has one or more of the following attributes:
(a) less than about 0.5% of the T cells are TCR+ T cells;
(b) less than about 2 ng of Cas9 or Cas12a per mL of the composition;
(c) less than about 0.05 fg of Cas9 or Cas12a per T cell;
(d) less than about 5×10¹¹ AAV6 capsids per mL of the composition;
(e) no verified off-target indel sites at a genomic locus other than TRAC and B2M;
(f) less than about 2.0% of total alleles have AAV6 integration at TRAC locus;
(g) less than about 1.0% of total alleles have AAV6 integration at B2M locus;
(h) undetectable AAV6 integration at loci other than TRAC and B2M; or
(i) less than about 900 ng of Strep-Tactin Multimer activation reagent per mL of the composition.

In any of the embodiments above relating to a composition comprising a population of T cells, the T cells can have the following attributes:
(i) at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% of the T cells are CD4+CD45RA+CCR7+;
(ii) less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, less than about 75%, less than about 80%, less than about 85%, less than about 90%, less than about 95%, or less than about 99% of the T cells are CD8+CD45RA-CCR7−;
(iii) less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, or less than about 25% of the T cells are CD3+aCAS3+;
(iv) at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% of the T cells are CD8+CCR7+; and
(v) at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% of the T cells are CD4+CD27+CD28+.

In any of the embodiments above relating to a composition comprising a population of T cells, the T cells can have the following attributes:
(i) at least about 13% to at least about 80% of the T cells are CD4+CD45RA+CCR7+;
(ii) less than about 3% to less than about 90% of the T cells are CD8+CD45RA-CCR7−;
(iii) less than about 1% to less than about 15% of the T cells are CD3+aCAS3+;
(iv) at least about 2% to at least about 93% of the T cells are CD8+CCR7+; and
(v) at least about 55% to at least about 98% of the T cells are CD4+CD27+CD28+.

In any of the embodiments above relating to a composition comprising a population of T cells, the T cells can have the following attributes:
(i) at least about 10% of the T cells are CD4+CD45RA+CCR7+;
(ii) less than about 25% of the T cells are CD8+CD45RA-CCR7−;
(iii) less than about 3.7% of the T cells are CD3+aCAS3+;
(iv) at least about 50% of the T cells are CD8+CCR7+; and
(v) at least about 89% of the T cells are CD4+CD27+CD28+.

In any of the embodiments above relating to a composition comprising a population of T cells, the attributes can be determined by analytical procedures known in the art. In certain aspects, the following attributes can be determined by analytical procedures:
(i) determining the % T cell viability by image cytometry cell counting;
(ii) determining the % T cells are CD2+CD5+ by flow cytometry;
(iii) determining the % T cells expressing CD19 CAR by flow cytometry;
(iv) determining the % T cells are HLA-I+ by flow cytometry;
(v) determining the ratio of % TCR+ to % CAR+ T cells by flow cytometry;
(vi) determining the % of total alleles having translocation between TRAC and B2M by polymerase chain reaction (PCR) or digital droplet PCR (ddPCR);
(vii) determining the number of viable CD2+CD5+CD19 CAR+ cells per mL of the composition by flow cytometry and/or image cytometry;
(viii) determining the % of total alleles having TRAC loci edited by ddPCR;
(ix) determining the % TCR+ T cells by flow cytometry;
(x) determining the % of total alleles having B2M loci edited by ddPCR;
(xi) determining the % of alleles having a nucleic acid encoding HLA-E(A), such as the HLA-E(A) having the amino acid sequence set forth in SEQ ID NO: 81 or SEQ ID NO: 132, knocked-in at B2M locus by ddPCR;
(xii) determining the % of alleles having a nucleic acid encoding CD19 CAR, such as a nucleic acid comprising the sequence set forth in SEQ ID NO: 136, or a nucleic acid encoding a CD19 CAR having an amino acid sequence set forth in SEQ ID NO: 78 or SEQ ID NO: 138, knocked-in at the TRAC locus by ddPCR;
(xiii) determining the amount of Cas9 or Cas12a per mL of the composition by ELISA;
(xiv) determining the amount of Cas9 or Cas12a per cell by dividing (the amount of Cas9 or Cas12a per mL of the composition by ELISA (numerator)) by (the number of cells per mL of the composition (denominator));
(xv) determining the amount of AAV6 capsids per mL of the composition by ELISA;
(xvi) determining the number of verified off-target indel sites at a genomic locus other than TRAC and B2M by next-generation sequencing (NGS);
(xvii) determining the % of total alleles having AAV6 integration at TRAC locus by ddPCR;
(xviii) determining the % of total alleles having AAV6 integration at B2M locus by ddPCR;
(xix) determining AAV6 integration at loci other than TRAC and B2M by NGS, such as a NGS-based method that identifies the genomic location of insertions of AAV6 inverted terminal repeats (ITRs);
(xx) determining the amount of Strep-Tactin Multimer activation reagent per mL of composition by ELISA;
(xxi) detecting bacterial growth by measuring the amount of CO₂ present in media;
(xxii) determining the amount of endotoxin by compendial testing, such as by the Limulus Amebocyte Lysate (LAL) Test;
(xxiii) detecting mycoplasma by PCR;
(xxiv) detecting cytokine-independent growth by a cell-based assay, such as by culturing cells in cytokine-free media and determining cell concentration and timepoints;
(xxv) detecting a significant unexpected karyotype by microscopy, such as by staining chromosomes and assessing them microscopically using G-banding technique; or
(xxvi) detecting HIV, HTLV, HAV, HBV, HCV, CMV, EBV, HHV6, HHV7, HHV8, and B19 by PCR, such as by RT-qPCR.

In any of the embodiments above relating to a composition comprising a population of T cells, frequency of TRAC indel, B2M indel, CD19 CAR knock-in, and HLA-E(A) knock-in can be determined by ddPCR using target-specific primers and probes, and a common external reference gene primer-probe set. In certain aspects, indel frequency can be determined using a first probe that binds an unedited region of the target and second probe that binds the edited region of the wild-type target but cannot bind to RNP-edited target sites due to nucleotide mismatch. The external reference primer-probe can be used to calculate total indels in the context of total genome copies. In certain embodiments, knock-in frequency can be determined using (i) a first primer-probe set that amplifies across the junction of the endogenous target locus and the inserted transgene to assess the percentage of knock-in, and (ii) a second primer-probe that binds and detects the CD19 CAR and/or HLA-E[A] insert to confirm its identity. In certain aspects, the external reference primer-probe set can be used to calculate the percentage of knock-in in the context of total genome copies.

In any of the embodiments above relating to a composition comprising a population of T cells, frequency of AAV integration can be determined using an NGS-based method that identifies the genomic location of insertions of specific AAV vector inverted terminal repeats (ITRs). In certain aspects, a method of determining frequency of AAV integration comprises (i) enzymatically fragmenting genomic DNA from paired edited and unedited donors and ligating custom Y-forked adapters to both ends of the DNA, (ii) subjecting end-repaired Y-adapter-ligated DNA fragments to a first round of PCR using ITR- and adapter-specific primers for targeted amplification of the genomic DNA junction and inserted vector ITR sequence, (iii) PCR amplifying adapters to each amplicon, and (iv) paired-end sequencing the library.

In any of the embodiments above relating to a composition comprising a population of T cells, cytokine-independent growth can be determined by the following assay in which Jurkat (cytokine-independent) cells serve as an assay positive-growth control, and NK92 (IL-2-dependent) cells serve as an assay negative-growth control. The viable cell concentration (VCC; cells/mL) of each sample population of T cells are assessed at day 14, 21, 31, 42, 56, and 70 and cells are counted in triplicate at each timepoint. Sample is deemed to pass the “no cytokine-independent growth” attribute and can be terminated on day 31, 42, 56, or 70 when the mean VCC of both replicate wells fall below the LoD. If the mean VCC of either one or both replicate well(s) are above LoD on day 70, the sample is deemed to fail the “no cytokine-independent growth” attribute. VCC of both assay controls are assessed on day 14, and the mean VCC of each replicate well conforms to assay acceptance criteria.

In any of the embodiments above relating to a composition comprising a population of T cells, detecting a significant unexpected karyotype by microscopy, such as by staining chromosomes and assessing them microscopically using G-banding technique, can be performed by the following method. Fifty cells are assessed in triplicate metaphase spreads for each sample population of T cells, and the occurrence and band identification of chromosomal rearrangements, aneuploidy, chromothripsis, and other aberrations are analyzed and quantitated. For each 50-cell sampling replicate each specific aberration or aberrant ploidy is expected to be present in <6 cells, the cut-off threshold for statistical significance based on Fisher's Exact test using a theoretical Zero Event sample as the reference. If at least 2 of 3 replicates do not contain any specific events greater than or equal to 6 cells, the acceptance criteria are met and results are deemed to pass the “no significant unexpected karyotype” attribute or “no specific aberration or aberrant ploidy observed above the limit” attribute.

In any of the embodiments above relating to a composition comprising a population of T cells, a dose of the composition can comprise about 25×10⁶ of the T cells. In certain embodiments, a dose of the composition can comprise about 30×10⁶ of the T cells. In certain embodiments, a dose of the composition can comprise about 35×10⁶ of the T cells. In certain embodiments, a dose of the composition can comprise about 40×10⁶ of the T cells. In certain embodiments, a dose of the composition can comprise about 45×10⁶ of the T cells. In certain embodiments, a dose of the composition can comprise about 50×10⁶ of the T cells. In certain embodiments, a dose of the composition can comprise about 55×10⁶ of the T cells. In certain embodiments, a dose of the composition can comprise about 60×10⁶ of the T cells. In certain embodiments, a dose of the composition can comprise about 65×10⁶ of the T cells. In certain embodiments, a dose of the composition can comprise about 70×10⁶ of the T cells. In certain embodiments, a dose of the composition can comprise about 75×10⁶ of the T cells. In certain embodiments, a dose of the composition can comprise about 100×10⁶ of the T cells. In certain embodiments, a dose of the composition can comprise about 150×10⁶ of the T cells. In certain embodiments, a dose of the composition can comprise about 200×10⁶ of the T cells. In certain embodiments, a dose of the composition can comprise about 250×10⁶ of the T cells. In certain embodiments, a dose of the composition can comprise about 300×10⁶ of the T cells. In certain embodiments, a dose of the composition can comprise about 350×10⁶ of the T cells. In certain embodiments, a dose of the composition can comprise about 400×10⁶ of the T cells. In certain embodiments, a dose of the composition can comprise about 450×10⁶ of the T cells. In certain embodiments, a dose of the composition can comprise about 500×10⁶ of the T cells. In certain embodiments, a dose of the composition can comprise about 550×10⁶ of the T cells. In certain embodiments, a dose of the composition can comprise about 600×10⁶ of the T cells. In certain embodiments, the dose is provided as a suspension for administration in a single intravenous (IV) infusion.

In any of the embodiments above relating to a composition comprising a population of T cells, the composition can comprise about 25×10⁶ of the T cells per mL of the composition. In certain embodiments, the composition can comprise about 30×10⁶ of the T cells per mL of the composition. In certain embodiments, the composition can comprise about 35×10⁶ of the T cells per mL of the composition. In certain embodiments, the composition can comprise about 40×10⁶ of the T cells per mL of the composition. In certain embodiments, the composition can comprise about 45×10⁶ of the T cells per mL of the composition. In certain embodiments, the composition can comprise about 50×10⁶ of the T cells per mL of the composition. In certain embodiments, the composition can comprise about 55×10⁶ of the T cells per mL of the composition. In certain embodiments, the composition can comprise about 60×10⁶ of the T cells per mL of the composition. In certain embodiments, the composition can comprise about 65×10⁶ of the T cells per mL of the composition. In certain embodiments, the composition can comprise about 70×10⁶ of the T cells per mL of the composition. In certain embodiments, the composition can comprise about 75×10⁶ of the T cells per mL of the composition. In certain embodiments, the composition can comprise about 100×10⁶ of the T cells per mL of the composition. In certain embodiments, the composition can comprise about 150×10⁶ of the T cells per mL of the composition. In certain embodiments, the composition can comprise about 200×10⁶ of the T cells per mL of the composition. In certain embodiments, the composition can comprise about 250×10⁶ of the T cells per mL of the composition. In certain embodiments, the composition can comprise about 300×10⁶ of the T cells per mL of the composition. In certain embodiments, the composition can comprise about 350×10⁶ of the T cells per mL of the composition. In certain embodiments, the composition can comprise about 400×10⁶ of the T cells per mL of the composition. In certain embodiments, the composition can comprise about 450×10⁶ of the T cells per mL of the composition. In certain embodiments, the composition can comprise about 500×10⁶ of the T cells per mL of the composition. In certain embodiments, the composition can comprise about 550×10⁶ of the T cells per mL of the composition. In certain embodiments, the composition can comprise about 600×10⁶ of the T cells per mL of the composition. In certain embodiments, the composition is provided as a suspension for administration in a single intravenous (IV) infusion.

### V. METHODS AND USES

Also provided are methods of using and uses of the provided genetically engineered T cells, and pharmaceutical compositions and formulations thereof, such as in the treatment of diseases, conditions, and disorders. Such methods and uses include therapeutic methods and uses, for example, involving administration of the genetically engineered T cells, or compositions containing the same, to a subject having a disease, condition, or disorder.

In some embodiments, the CAR of the genetically engineered T cell is directed against CD19, and the diseases, conditions and disorders include any in which CD19 is expressed. Such methods and uses include therapeutic methods and uses, for example, involving administration of the genetically engineered T cells to a subject having a disease, condition, or disorder associated with CD19 such as a disease, condition, or disorder associated with CD19 expression, and/or in which cells or tissues express, e.g., specifically express, CD19. Such methods and uses include therapeutic methods and uses, for example, involving administration of the genetically engineered T cells, or compositions containing the same, to a subject having a disease, condition, or disorder associated with CD19 such as a disease, condition, or disorder associated with CD19 expression, and/or in which cells or tissues express, e.g., specifically express, CD19.

In some embodiments, the genetically engineered T cells are administered in an effective amount to effect treatment of the disease or disorder. Provided herein are uses of the recombinant receptors (e.g., CARs), and cells (e.g., engineered cells) in such methods and treatments, and in the preparation of a medicament in order to carry out such therapeutic methods. In some embodiments, the methods are carried out by administering the binding molecules or cells, or compositions comprising the same, to the subject having, having had, or suspected of having the disease or condition. In some embodiments, the methods thereby treat the disease or condition or disorder in the subject. Also provided herein are use of any of the compositions, such as pharmaceutical compositions provided herein, for the treatment of a disease or disorder associated with CD19, such as use in a treatment regimen. Also provided herein are use of any of the compositions, such as pharmaceutical compositions provided herein, for the treatment of a disease or disorder associated with CD19, such as use in a treatment regimen. Also provided herein are use of any of the compositions, such as pharmaceutical compositions provided herein, for the treatment of a disease or disorder associated with CD19, such as use in a treatment regimen.

Provided herein are methods of treatment that involve administering engineered cells or compositions containing engineered cells, such as engineered T cells, including methods for the treatment of subjects with systemic autoimmune diseases. In some embodiments, provided herein are methods and use of genetically engineered cells t cells (e.g., CD19-directed CAR engineered T cells) and/or compositions thereof, including methods for the treatment of subjects with systemic autoimmune diseases. In some embodiments, the systemic autoimmune disease is a severe and/or moderate systemic autoimmune diseases that have failed at least two or more prior therapies. In particular embodiments, the method includes administering to the subject a dose of T cells that includes CD4+ and CD8+ T cells, wherein the T cells comprises a chimeric antigen receptor (CAR) that specifically binds to an antigen associated with cells of the condition. In some embodiments, the CAR specifically binds to CD19. In particular embodiments, the method includes administering to the subject a dose of T cells that includes CD4+ and CD8+ T cells, wherein the T cells comprises a chimeric antigen receptor (CAR) that specifically binds to CD19.

In some embodiment, the methods provided herein are used to treat autoimmune diseases caused by, associated with and/or specific to cells expressing an antigen targeted by the CAR, such as CD19. In some embodiment, the methods provided herein are used to treat autoimmune diseases caused by, associated with and/or specific to cells expressing CD19.

In some embodiments, the autoimmune disease include, but are not limited to, Addison's disease, allergies, ankylosing spondylitis, asthma, atherosclerosis, autoimmune diseases of the ear, autoimmune diseases of the eye, autoimmune hepatitis, autoimmune parotitis, colitis, coronary heart disease, diabetes, including Type 1 and/or Type 2 diabetes, epididymitis, glomerulonephritis, Graves' disease, Guillain-Barre syndrome, Hashimoto's disease, hemolytic anemia, idiopathic thrombocytopenic purpura, inflammatory bowel disease, immune response to recombinant drug products, myasthenia gravis, pemphigus, psoriasis, rheumatic fever, rheumatoid arthritis, sarcoidosis, scleroderma, spondyloarthropathies, thyroiditis, transplant rejection, vasculitis, AIDS, atopic allergy, bronchial asthma, eczema, leprosy, schizophrenia, chronic fatigue syndrome, Alzheimer's disease, Parkinson's disease, myocardial infarction, stroke, autism, epilepsy, Arthus's phenomenon, and anaphylaxis.

In some embodiments, the systemic autoimmune diseases include systemic lupus erythematosus (SLE) and severe SLE, rheumatoid arthritis (RA), and systemic sclerosis (SSc). In some embodiments, the systemic autoimmune diseases may include Systemic Lupus Erythematosus (SLE), Sjogren's' syndrome, progressive systemic sclerosis (i.e., scleroderma), idiopathic inflammatory myositis (IIM, including dermatomyositis, polymyositis and necrotizing myositis), mixed connective tissue disorder (MCTD), relapsing-remitting multiple sclerosis, ANCA-associated vasculitis (AAV), Crohn's disease, myasthenia gravis, Behçet's, rheumatoid arthritis, multiple sclerosis, primary progressive MS, IgA nephropathy, pemphigus vulgaris, myasthernia gravis, autoimmune hemolytic anemia, immune thrombocytopenia, IgG4-related diseases, membranous nephropathy, cutaneous lupus erythematosus, sarcoidosis, light chain amyloidosis, acute respiratory distress syndrome, atopic eczema, hereditary angioedema, hidradenitis suppurative, inclusion-body myositis, inflammatory bowel disease, mastocytosis, multifocal motor neuropathy, necrotizing myopathy, neuromyelitis optica spectrum disorder, mixed connective tissue disorder, POEMS syndrome, primary biliary cholangitis, psoriasis, rhesus hemolytic disease, Still's disease, type 1 diabetes, urticaria, capillary leakage syndrome, cytokine release syndrome, erythema multiforme, pyoderma gangrenosum, x-linked agammaglobulinemia, antiphospholipid syndrome, and chronic inflammatory demyelinating polyneuropathy.and chronic inflammatory demyelinating polyneuropathy.

In some embodiment, the methods provided herein are used to treat autoimmune diseases such as, SLE, IMM, SSc, AAV, systemic sclerosis, multiple sclerosis, highly active replapsing remitting multiple sclerosis (MS), primary progressive MS, IgA nephropathy, pemphigus vulgaris, myasthernia gravis, demyelinating polyradiculoneuropathy, autoimmune hemolytic anemia, immune thrombocytopenia, IgG4-related diseases, membranous nephropathy, Primary Sjorgren's Syndrom, cutaneous lupus erythematosus, sarcoidosis, light chain amyloidosis, rheumatoid arthritis, bullous pemphigoid, acute respiratory distress syndrome, atopic eczema, hereditary angioedema, hidradenitis suppurative, inclusion-body myositis, inflammatory bowel disease, mastocytosis, multifocal motor neuropathy, necrotizing myopathy, neuromyelitis optica spectrum disorder, mixed connective tissue disorder, POEMS syndrome, primary biliary cholangitis, psoriasis, rhesus hemolytic disease, Still's disease, type 1 diabetes, urticaria, capillary leakage syndrome, cytokine release syndrome, erythema multiforme, pyoderma gangrenosum, antiphospholipid syndrome, or x-linked agammaglobulinemia. In some embodiments, the methods provided herein are used to treat of SLE, IM, AAV, systemic sclerosis, highly active replapsing remitting multiple sclerosis (MS), primary progressive MS, IgA nephropathy, pemphigus vulgaris, or myasthernia gravis. In some embodiments, the methods provided herein are used to treate SLE. In some embodiments, the methods provided herein are used to treate IIM. In some embodiments, the methods provided herein are used to treate SSc. In some embodiments, the methods provided herein are used to treat MS.

In some embodiments, the systemic autoimmune disease is SLE, such as a moderate SLE or severe refractory SLE, idiopathic inflammatory myopathy, systemic sclerosis, rheumatoid arthritis (RA), or multiple sclerosis. Among provided methods are methods of treatment that involve administering engineered cells or compositions containing engineered cells, such as engineered T cells to subjects with SLE, including severe refractory SLE. Also provided are methods and uses of provided CD19-directed CAR engineered cells (e.g., T cells) and/or compositions thereof, including methods for the treatment of subjects having a SLE, including severe refractory SLE, that involves administration of the engineered cells and/or compositions thereof. In certain embodiments, the subject has severe refractory SLE. In some embodiments, the subject is selected for or identified as having severe refractory SLE, such as by the presence of certain features or clinical manifestations that indicate the presence of severe refractory SLE. In some embodiments, the methods and use of provided genetically engineered T cells, and compositions thereof, include methods for the treatment of subjects with severe refractory SLE that have failed at least two or more prior therapies. In particular embodiments, the method includes administering to the subject a dose of T cells that includes CD4+ and CD8+ T cells, wherein the T cells comprise a chimeric antigen receptor (CAR) that specifically binds to an antigen associated with the SLE, such as CD19.

In some embodiments, the disease or disorder is a B cell-related disorder. In some of any of the provided embodiments of the provided methods, the disease or disorder is an autoimmune disease or disorder. In some of any of the provided embodiments of the provided methods, the autoimmune disease or disorder is systemic lupus erythematosus (SLE), lupus nephritis, inflammatory bowel disease, rheumatoid arthritis, ANCA associated vasculitis, idiopathic thrombocytopenia purpura (ITP), thrombotic thrombocytopenia purpura (TTP), autoimmune thrombocytopenia, Chagas' disease, Grave's disease, Wegener's granulomatosis, poly-arteritis nodosa, Sjogren's syndrome, pemphigus vulgaris, scleroderma, Crohn's disease, asthma, multiple sclerosis, psoriasis, IgA nephropathy, IgM polyneuropathies, vasculitis, diabetes mellitus, Reynaud's syndrome, anti-phospholipid syndrome, Goodpasture's disease, Kawasaki disease, autoimmune hemolytic anemia, myasthenia gravis, progressive glomerulonephritis, and/or a disease or condition associated with transplant.

In some embodiments, the disease, disorder or condition to be treated is a tumor, cancer, malignancy, neoplasm, or other proliferative disease or disorder. In some embodiments, the disease or condition is a B cell malignancy. Such diseases include but are not limited to leukemia, lymphoma, and multiple myeloma (MM). In some embodiments, the B cell malignancy is selected from among acute lymphoblastic leukemia (ALL), adult ALL, pro-lymphocytic leukemias, hairy cell leukemias, small lymphocytic lymphoma (SLL), common acute lymphocytic leukemias, chronic lymphoblastic leukemia (CLL), Null-acute lymphoblastic leukemias, follicular lymphoma, splenic lymphoma, marginal zone lymphoma, mantle cell lymphoma, indolent B cell lymphoma, Anaplastic large cell lymphoma (ALCL), Hodgkin lymphoma, non-Hodgkin lymphoma (NHL), and Diffuse Large B-Cell Lymphoma (DLBCL). In some embodiments, the disease or condition is NHL. In some embodiments, the NHL is selected from the group consisting of aggressive NHL, diffuse large B cell lymphoma (DLBCL), NOS (de novo and transformed from indolent), primary mediastinal large B cell lymphoma (PMBCL), T cell/histocyte-rich large B cell lymphoma (TCHRBCL), Burkitt's lymphoma, mantle cell lymphoma (MCL), and/or follicular lymphoma (FL), or refractory follicular lymphoma, optionally, follicular lymphoma Grade 3B (FL3B).

### VI. DOSES AND REGIMENS

In certain embodiments, a dose of a composition comprising a plurality of T cells described herein comprises about 25×10⁶ of the T cells. In certain embodiments, a dose of a composition comprising a plurality of T cells described herein comprises about 50×10⁶ of the T cells. In certain embodiments, a dose of a composition comprising a plurality of T cells described herein comprises about 100×10⁶ of the T cells. In certain embodiments, a dose of a composition comprising a plurality of T cells described herein comprises about 150×10⁶ of the T cells. In certain embodiments, a dose of a composition comprising a plurality of T cells described herein comprises about 200×10⁶ of the T cells. In certain embodiments, a dose of a composition comprising a plurality of T cells described herein comprises about 250×10⁶ of the T cells. In certain embodiments, a dose of a composition comprising a plurality of T cells described herein comprises about 300×10⁶ of the T cells. In certain embodiments, a dose of a composition comprising a plurality of T cells described herein comprises about 350×10⁶ of the T cells. In certain embodiments, a dose of a composition comprising a plurality of T cells described herein comprises about 400×10⁶ of the T cells. In certain embodiments, a dose of a composition comprising a plurality of T cells described herein comprises about 450×10⁶ of the T cells. In certain embodiments, a dose of a composition comprising a plurality of T cells described herein comprises about 500×10⁶ of the T cells. In certain embodiments, a dose of a composition comprising a plurality of T cells described herein comprises about 550×10⁶ of the T cells. In certain embodiments, a dose of a composition comprising a plurality of T cells described herein comprises about 600×10⁶ of the T cells. In certain embodiments, the dose is provided as a suspension for administration in a single intravenous (IV) infusion.

In certain embodiments, a method of treating autoimmune disease in a subject comprises administering to the subject a composition comprising about 25×10⁶ T cells described herein. In certain embodiments, a method of treating autoimmune disease in a subject comprises administering to the subject a composition comprising about 50×10⁶ T cells described herein. In certain embodiments, a method of treating autoimmune disease in a subject comprises administering to the subject a composition comprising about 100×10⁶ T cells described herein. In certain embodiments, a method of treating autoimmune disease in a subject comprises administering to the subject a composition comprising about 150×10⁶ T cells described herein. In certain embodiments, a method of treating autoimmune disease in a subject comprises administering to the subject a composition comprising about 200×10⁶ T cells described herein. In certain embodiments, a method of treating autoimmune disease in a subject comprises administering to the subject a composition comprising about 250×10⁶ T cells described herein. In certain embodiments, a method of treating autoimmune disease in a subject comprises administering to the subject a composition comprising about 300×10⁶ T cells described herein. In certain embodiments, a method of treating autoimmune disease in a subject comprises administering to the subject a composition comprising about 350×10⁶ T cells described herein. In certain embodiments, a method of treating autoimmune disease in a subject comprises administering to the subject a composition comprising about 400×10⁶ T cells described herein. In certain embodiments, a method of treating autoimmune disease in a subject comprises administering to the subject a composition comprising about 450×10⁶ T cells described herein. In certain embodiments, a method of treating autoimmune disease in a subject comprises administering to the subject a composition comprising about 500×10⁶ T cells described herein. In certain embodiments, a method of treating autoimmune disease in a subject comprises administering to the subject a composition comprising about 550×10⁶ T cells described herein. In certain embodiments, a method of treating autoimmune disease in a subject comprises administering to the subject a composition comprising about 600×10⁶ T cells described herein. In certain embodiments, about 25×10⁶′ about 30×10⁶, about 35×10⁶, about 40×10⁶, about 45×10⁶, about 50×10⁶, about 55×10⁶, about 60×10⁶, 65×10⁶, about 70×10⁶, about 75×10⁶, about 80×10⁶, about 85×10⁶, about 90×10⁶, about 95×10⁶, about 100×10⁶, about 125×10⁶, about 150×10⁶, about 175×10⁶, about 200×10⁶, about 225×10⁶, about 250×10⁶, about 275×10⁶, about 300×10⁶, about 325×10⁶, about 350×10⁶, about 375×10⁶, about 400×10⁶, about 425×10⁶, about 450×10⁶, about 475×10⁶, about 500×10⁶, about 525×10⁶, about 550×10⁶, about 575×10⁶, or about 600×10⁶ T cells are administered to the subject. In certain embodiments, the autoimmune disease is systemic lupus erythematosus (SLE), idiopathic inflammatory myopathy (IIM), systemic sclerosis (SSc), or rheumatoid arthritis (RA). In certain embodiments, the T cells are administered to the subject in a single intravenous (IV) infusion.

In certain embodiments, a method of treating systemic lupus erythematosus (SLE) in a subject comprises administering to the subject a composition comprising about 25×10⁶ T cells described herein, a composition comprising about 50×10⁶ T cells described herein, a composition comprising about 100×10⁶ T cells described herein, a composition comprising about 150×10⁶ T cells described herein, a composition comprising about 200×10⁶ T cells described herein, a composition comprising about 250×10⁶ T cells described herein, a composition comprising about 300×10⁶ T cells described herein, a composition comprising about 350×10⁶ T cells described herein, a composition comprising about 400×10⁶ T cells described herein, a composition comprising about 450×10⁶ T cells described herein, a composition comprising about 500×10⁶ T cells described herein, a composition comprising about 550×10⁶ T cells described herein, or a composition comprising about 600×10⁶ T cells described herein. In certain embodiments, about 25×10⁶′ about 30×10⁶, about 35×10⁶, about 40×10⁶, about 45×10⁶, about 50×10⁶, about 55×10⁶, about 60×10⁶, 65×10⁶, about 70×10⁶, about 75×10⁶, about 80×10⁶, about 85×10⁶, about 90×10⁶, about 95×10⁶, about 100×10⁶, about 125×10⁶, about 150×10⁶, about 175×10⁶, about 200×10⁶, about 225×10⁶, about 250×10⁶, about 275×10⁶, about 300×10⁶, about 325×10⁶, about 350×10⁶, about 375×10⁶, about 400×10⁶, about 425×10⁶, about 450×10⁶, about 475×10⁶, about 500×10⁶, about 525×10⁶, about 550×10⁶, about 575×10⁶, or about 600×10⁶ T cells are administered to the subject. In certain embodiments, administering the aforementioned compositions results in remission of SLE according to DORIS definition, achievement of Lupus Low Disease Activity State (LLDAS), an improvement in proteinuria, and/or an improvement in Health Assessment Questionnaire-Disability Index (HAQ-DI). In certain aspects, remission of SLE according to DORIS definition is the criteria described in van Vollenhoven R F, Bertsias G, Doria A, et al. 2021 DORIS definition of remission in SLE: Final recommendations from an international task force, Lupus Sci Med 2022; 9: e000634; Lupus Sci Med. 2021; 8:e000538, which is incorporated by reference in its entirety. In certain embodiments, remission of SLE according to DORIS is the criteria is based on the Systemic Lupus Erythematosus Disease Activity Index-2000 (SLEDAI-2K), the Physician's Global Assessment of Disease Activity (PhGA) (0-3), and achieving stable lupus-specific therapies irrespective of persistent serology. In certain embodiments, SLE remission according to DORIS definition is the 2021 DORIS definition of remission. In certain embodiments, remission of SLE comprises a) Clinical SLEDAI=0; b) PhGA<0.5 (from the 0-3 visual analog scale (VAS) scale) irrespective of serologies; and c) the subject may be on antimalarials, low-dose glucocorticoids (prednisolone≤5 mg/day), and/or stable immunosuppressives including biologics. In certain embodiments, LLDAS is the following criteria: (1) SLEDAI-2K≤4, with no activity in major organ systems (renal, CNS, cardiopulmonary, vasculitis, fever) and no hemolytic anemia or gastrointestinal activity; (2) No new lupus disease activity compared with the previous assessment; (3) A Safety of Estrogens in Lupus Erythematosus National Assessment (SELENA)-SLEDAI physician global assessment (scale 0 to 3)≤1; (4) A current prednisolone (or equivalent) dose≤7.5 mg daily; and/or (5) well-tolerated standard maintenance doses of immunosuppressive drugs and approved biological agents. In certain embodiments, the HAQ-DI comprises 20 questions in 8 categories of functioning, which represent a comprehensive set of functional activities—dressing, rising, eating, walking, hygiene, reach, grip, and usual activities, wherein the questions asks the subjects if they are able to perform a particular task and the subjects' responses are made on a scale from 0 (no disability) to 3 (completely disabled). In certain embodiments, the T cells are administered to the subject in a single intravenous (IV) infusion.

In certain embodiments, a method of treating systemic sclerosis (SSc) in a subject comprises administering to the subject a composition comprising about 25×10⁶ T cells described herein, a composition comprising about 50×10⁶ T cells described herein, a composition comprising about 100×10⁶ T cells described herein, a composition comprising about 150×10⁶ T cells described herein, a composition comprising about 200×10⁶ T cells described herein, a composition comprising about 250×10⁶ T cells described herein, a composition comprising about 300×10⁶ T cells described herein, a composition comprising about 350×10⁶ T cells described herein, a composition comprising about 400×10⁶ T cells described herein, a composition comprising about 450×10⁶ T cells described herein, a composition comprising about 500×10⁶ T cells described herein, a composition comprising about 550×10⁶ T cells described herein, or a composition comprising about 600×10⁶ T cells described herein. In certain embodiments, about 25×10⁶′ about 30×10⁶, about 35×10⁶, about 40×10⁶, about 45×10⁶, about 50×10⁶, about 55×10⁶, about 60×10⁶, 65×10⁶, about 70×10⁶, about 75×10⁶, about 80×10⁶, about 85×10⁶, about 90×10⁶, about 95×10⁶, about 100×10⁶, about 125×10⁶, about 150×10⁶, about 175×10⁶, about 200×10⁶, about 225×10⁶, about 250×10⁶, about 275×10⁶, about 300×10⁶, about 325×10⁶, about 350×10⁶, about 375×10⁶, about 400×10⁶, about 425×10⁶, about 450×10⁶, about 475×10⁶, about 500×10⁶, about 525×10⁶, about 550×10⁶, about 575×10⁶, or about 600×10⁶ T cells are administered to the subject. In certain embodiments, administering the aforementioned compositions results in the subject achieving a minimal clinically important differences (MCID) of 24%, an improvement from baseline of the modified Rodnan Skin Score (mRSS), an improvement in HAQ-DI, and/or an improvement from baseline of the Revised CRISS. In certain embodiments, the mRSS comprises measuring skin thickness. In certain embodiments, the improvement from baseline of the Revised CRISS comprises improvement in at least one, at least two, at least three, at least four, or five core set measures: (1) modified Rodnan skin score (mRSS), (2) percent predicted forced vital capacity (FVC %), (3) health assessment questionnaire-disability index (HAQ-DI), (4) patient global assessments (PtGA), and (5) physician global assessments (PhGA). Details of the Revised Criss are described in Khanna D., et al. New composite endpoint in early diffuse cutaneous systemic sclerosis: revisiting the provisional American College of Rheumatology Composite Response Index in Systemic Sclerosis [published correction appears in Ann Rheum Dis 2021; 80:e154]. Ann Rheum Dis 2021; 80:641-50, which is incorporated by reference in its entirety. In certain embodiments, the T cells are administered to the subject in a single intravenous (IV) infusion.

In certain embodiments, a method of treating idiopathic inflammatory myopathy (IIM) in a subject comprises administering to the subject a composition comprising about 25×10⁶ T cells described herein, a composition comprising about 50×10⁶ T cells described herein, a composition comprising about 100×10⁶ T cells described herein, a composition comprising about 150×10⁶ T cells described herein, a composition comprising about 200×10⁶ T cells described herein, a composition comprising about 250×10⁶ T cells described herein, a composition comprising about 300×10⁶ T cells described herein, a composition comprising about 350×10⁶ T cells described herein, a composition comprising about 400×10⁶ T cells described herein, a composition comprising about 450×10⁶ T cells described herein, a composition comprising about 500×10⁶ T cells described herein, a composition comprising about 550×10⁶ T cells described herein, or a composition comprising about 600×10⁶ T cells described herein. In certain embodiments, about 25×10⁶′ about 30×10⁶, about 35×10⁶, about 40×10⁶, about 45×10⁶, about 50×10⁶, about 55×10⁶, about 60×10⁶, 65×10⁶, about 70×10⁶, about 75×10⁶, about 80×10⁶, about 85×10⁶, about 90×10⁶, about 95×10⁶, about 100×10⁶, about 125×10⁶, about 150×10⁶, about 175×10⁶, about 200×10⁶, about 225×10⁶, about 250×10⁶, about 275×10⁶, about 300×10⁶, about 325×10⁶, about 350×10⁶, about 375×10⁶, about 400×10⁶, about 425×10⁶, about 450×10⁶, about 475×10⁶, about 500×10⁶, about 525×10⁶, about 550×10⁶, about 575×10⁶, or about 600×10⁶ T cells are administered to the subject. In certain embodiments, administering the aforementioned compositions results in an improvement of Myositis Response Criteria (MRC) Total Improvement Score (TIS) (MRC-TIS), an improvement in HAQ-DI, and/or an improvement in Cutaneous Dermatomyositis Disease Area and Severity Index (CDASI). In certain embodiments, improvement in MRC-TIS comprises an improvement in MMT-8, PhGA (0 to 10), PtGA (0 to 10), HAQ-DI, MDAAT Extramuscular Global Assessment and creatine kinase (CK). In certain embodiments, improvement in MRC-TIS comprises at least a 20 point increase, at least a 40 point increase, or at least a 60 point increase in the IMACS definition of improvement based on MMT-8, PhGA (0 to 10), PtGA (0 to 10), HAQ-DI, MDAAT Extramuscular Global Assessment and creatine kinase (CK). Details of MRC-TIS and IMACS are described in Rider LG., et al. Defining clinical improvement in adult and juvenile myositis. J Rheumatol 2003; 30:603-17, which is incorporated by reference in its entirety. In certain embodiments, the T cells are administered to the subject in a single intravenous (IV) infusion.

In certain embodiments, a method of treating rheumatoid arthritis (RA) in a subject comprises administering to the subject a composition comprising about 25×10⁶ T cells described herein, a composition comprising about 50×10⁶ T cells described herein, a composition comprising about 100×10⁶ T cells described herein, a composition comprising about 150×10⁶ T cells described herein, a composition comprising about 200×10⁶ T cells described herein, a composition comprising about 250×10⁶ T cells described herein, a composition comprising about 300×10⁶ T cells described herein, a composition comprising about 350×10⁶ T cells described herein, a composition comprising about 400×10⁶ T cells described herein, a composition comprising about 450×10⁶ T cells described herein, a composition comprising about 500×10⁶ T cells described herein, a composition comprising about 550×10⁶ T cells described herein, or a composition comprising about 600×10⁶ T cells described herein. In certain embodiments, about 25×10⁶′ about 30×10⁶, about 35×10⁶, about 40×10⁶, about 45×10⁶, about 50×10⁶, about 55×10⁶, about 60×10⁶, 65×10⁶, about 70×10⁶, about 75×10⁶, about 80×10⁶, about 85×10⁶, about 90×10⁶, about 95×10⁶, about 100×10⁶, about 125×10⁶, about 150×10⁶, about 175×10⁶, about 200×10⁶, about 225×10⁶, about 250×10⁶, about 275×10⁶, about 300×10⁶, about 325×10⁶, about 350×10⁶, about 375×10⁶, about 400×10⁶, about 425×10⁶, about 450×10⁶, about 475×10⁶, about 500×10⁶, about 525×10⁶, about 550×10⁶, about 575×10⁶, or about 600×10⁶ T cells are administered to the subject. In certain embodiments, administering the aforementioned compositions results in an improvement in the Disease Activity Score 28-CRP (DAS-28) and/or an improvement in HAQ-DI. Details of DAS-28 are described in Greenmyer JR., et al. DAS28-CRP Cutoffs for High Disease Activity and Remission Are Lower Than DAS28-ESR in Rheumatoid Arthritis. ACR Open Rheumatol. 2020 September; 2(9):507-511, which is incorporated by reference in its entirety. In certain embodiments, the T cells are administered to the subject in a single intravenous (IV) infusion.

In certain embodiments, a method of treating cancer in a subject comprises administering to the subject a composition comprising about 25×10⁶ T cells described herein. In certain embodiments, a method of treating cancer in a subject comprises administering to the subject a composition comprising about 50×10⁶ T cells described herein. In certain embodiments, a method of treating cancer in a subject comprises administering to the subject a composition comprising about 100×10⁶ T cells described herein. In certain embodiments, a method of treating cancer in a subject comprises administering to the subject a composition comprising about 150×10⁶ T cells described herein. In certain embodiments, a method of treating cancer in a subject comprises administering to the subject a composition comprising about 200×10⁶ T cells described herein. In certain embodiments, a method of treating cancer in a subject comprises administering to the subject a composition comprising about 250×10⁶ T cells described herein. In certain embodiments, a method of treating cancer in a subject comprises administering to the subject a composition comprising about 300×10⁶ T cells described herein. In certain embodiments, a method of treating cancer in a subject comprises administering to the subject a composition comprising about 350×10⁶ T cells described herein. In certain embodiments, a method of treating cancer in a subject comprises administering to the subject a composition comprising about 400×10⁶ T cells described herein. In certain embodiments, a method of treating cancer in a subject comprises administering to the subject a composition comprising about 450×10⁶ T cells described herein. In certain embodiments, a method of treating cancer in a subject comprises administering to the subject a composition comprising about 500×10⁶ T cells described herein. In certain embodiments, a method of treating cancer in a subject comprises administering to the subject a composition comprising about 550×10⁶ T cells described herein. In certain embodiments, a method of treating cancer in a subject comprises administering to the subject a composition comprising about 600×10⁶ T cells described herein. In certain embodiments, about 25×10⁶, about 30×10⁶, about 35×10⁶, about 40×10⁶, about 45×10⁶, about 50×10⁶, about 55×10⁶, about 60×10⁶, 65×10⁶, about 70×10⁶, about 75×10⁶, about 80×10⁶, about 85×10⁶, about 90×10⁶, about 95×10⁶, about 100×10⁶, about 125×10⁶, about 150×10⁶, about 175×10⁶, about 200×10⁶, about 225×10⁶, about 250×10⁶, about 275×10⁶, about 300×10⁶, about 325×10⁶, about 350×10⁶, about 375×10⁶, about 400×10⁶, about 425×10⁶, about 450×10⁶, about 475×10⁶, about 500×10⁶, about 525×10⁶, about 550×10⁶, about 575×10⁶, or about 600×10⁶ T cells are administered to the subject. In certain embodiments, the cancer is a B cell malignancy. In certain embodiments, the cancer is leukemia, lymphoma, or multiple myeloma (MM). In some embodiments, the B cell malignancy is selected from among acute lymphoblastic leukemia (ALL), adult ALL, pro-lymphocytic leukemias, hairy cell leukemias, small lymphocytic lymphoma (SLL), common acute lymphocytic leukemias, chronic lymphoblastic leukemia (CLL), Null-acute lymphoblastic leukemias, follicular lymphoma, splenic lymphoma, marginal zone lymphoma, mantle cell lymphoma, indolent B cell lymphoma, Anaplastic large cell lymphoma (ALCL), Hodgkin lymphoma, non-Hodgkin lymphoma (NHL), and Diffuse Large B-Cell Lymphoma (DLBCL). In certain embodiments, the T cells are administered to the subject in a single intravenous (IV) infusion.

### VII. ARTICLES OF MANUFACTURE OR KITS

Also provided are articles of manufacture or kits containing the provided recombinant receptors (e.g., CARs), genetically engineered cells, and/or compositions comprising the same. The articles of manufacture may include a container and a label or package insert on or associated with the container. Suitable containers include, for example, bottles, vials, syringes, test tubes, IV solution bags, etc. The containers may be formed from a variety of materials such as glass or plastic. In some embodiments, the container has a sterile access port. Exemplary containers include an intravenous solution bags, vials, including those with stoppers pierceable by a needle for injection. The article of manufacture or kit may further include a package insert indicating that the compositions can be used to treat a particular condition such as a condition described herein (e.g., a cancer). Alternatively, or additionally, the article of manufacture or kit may further include another or the same container comprising a pharmaceutically-acceptable buffer. It may further include other materials such as other buffers, diluents, filters, needles, and/or syringes.

The label or package insert may indicate that the composition is used for treating the CD19-expressing or CD19-associated disease, disorder or condition in an individual. The label or a package insert, which is on or associated with the container, may indicate directions for reconstitution and/or use of the formulation. The label or package insert may further indicate that the formulation is useful or intended for subcutaneous, intravenous, or other modes of administration for treating or preventing a CD19-expressing or CD19-associated disease, disorder or condition in an individual.

The container in some embodiments holds a composition which is by itself or combined with another composition effective for treating, preventing and/or diagnosing the condition. The article of manufacture or kit may include (a) a first container with a composition contained therein (i.e., first medicament), wherein the composition includes the CAR; and (b) a second container with a composition contained therein (i.e., second medicament), wherein the composition includes a further agent, such as a cytotoxic or otherwise therapeutic agent, and which article or kit further comprises instructions on the label or package insert for treating the subject with the second medicament, in an effective amount.

### VIII. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

As used herein, the term “endogenous” refers to a referenced molecule, such as a polynucleotide (e.g. gene), or polypeptide, that is present in a native or unmodified cell, such as a T cell. For instance, the term when used in reference to expression of an endogenous gene refers to expression of a gene encoded by an endogenous nucleic acid contained within a T cell isolated from a subject.

As used herein, a “gene,” includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions. The sequence of a gene is typically present at a fixed chromosomal position or locus on a chromosome in the cell.

As used herein, the term “locus” or “loci” refers to a fixed position on a chromosome where a particular gene or genetic marker is located. Reference to a “gene loci” refers to a particular locus of a desired gene in which it is desired to target a genetic modification, such as a gene edit or integration of a transgene.

As used herein “genetic disruption” with reference to an endogenous gene loci refers to a loci that has been modified using gene editing for directed DNA cleavage so that no functional endogenous gene product is produced from the disrupted genetic loci. In certain embodiments, the disruption results in expression of non-functional protein products, including but not limited to truncations, deletions, point mutations and insertions. In other embodiments, the disruption results in no protein expression from the endogenous gene loci.

As used herein, the term “expression” with reference to a gene or “gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or can be a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristoylation, and glycosylation. Hence, reference to expression or gene expression includes protein (or polypeptide) expression or expression of a transcribable product of a gene such as mRNA. The protein expression may include intracellular expression or surface expression of a protein. Typically, expression of a gene product, such as mRNA or protein, is at a level that is detectable in the cell.

As used herein, a “detectable” expression level, means a level that is detectable by standard techniques known to a skilled artisan, and include for example, differential display, RT (reverse transcriptase)-coupled polymerase chain reaction (PCR), Northern Blot, and/or RNase protection analyses as well as immunoaffinity-based methods for protein detection, such as flow cytometry, ELISA, or western blot. The degree of expression levels need only be large enough to be visualized or measured via standard characterization techniques.

As used herein, the term “transgene” with reference to a polynucleotide is intended to mean that the referenced molecule is introduced into the cell of interest such that it is transferred into the cell. Typically, a transgene sequence is not normally expressed by the cells. In some cases, the transgene sequence is thus heterologous or exogenous to the cell and introduced, for example, by introduction of an exogenous encoding nucleic acid into the genetic material of the cells such as by integration into a chromosome or as non-chromosomal genetic material such as a plasmid or expression vector. Therefore, the term as it is used in reference to expression of an encoding nucleic acid refers to introduction of the encoding nucleic acid in an expressible form into the cell.

In some embodiments, “operably linked” may include the association of components, such as a DNA sequence, e.g. a heterologous nucleic acid) and a regulatory sequence(s), in such a way as to permit gene expression when the appropriate molecules (e.g. transcriptional activator proteins) are bound to the regulatory sequence. Hence, it means that the components described are in a relationship permitting them to function in their intended manner.

The terms “polypeptide” and “protein” are used interchangeably to refer to a polymer of amino acid residues, and are not limited to a minimum length. Polypeptides, including the antibodies and antibody chains and other peptides, e.g., linkers, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

As used herein, “percent (%) amino acid sequence identity” and “percent identity” and “sequence identity” when used with respect to an amino acid sequence (reference polypeptide sequence) is defined as the percentage of amino acid residues in a candidate sequence (e.g., the subject antibody or fragment) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

An amino acid substitution may include replacement of one amino acid in a polypeptide with another amino acid. Amino acid substitutions may be introduced into a binding molecule, e.g., antibody, of interest and the products screened for a desired activity, e.g., retained/improved antigen binding, or decreased immunogenicity.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, the singular forms “a,” “an,” and “the” include plural referents unless the context clearly dictates otherwise. For example, “a” or “an” means “at least one” or “one or more.” It is understood that aspects, embodiments, and variations described herein include “comprising,” “consisting,” and/or “consisting essentially of” aspects, embodiments and variations.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

“Preventing,” as used herein, includes providing prophylaxis with respect to the occurrence or recurrence of a disease in a subject that may be predisposed to the disease but has not yet been diagnosed with the disease. In some embodiments, the provided molecules and compositions are used to delay development of a disease or to slow the progression of a disease.

An “effective amount” of an agent, e.g., a pharmaceutical formulation, binding molecule, antibody, cells, or composition, in the context of administration, refers to an amount effective, at dosages/amounts and for periods of time necessary, to achieve a desired result, such as a therapeutic or prophylactic result.

As used herein, a “subject” or an “individual” is a mammal. In some embodiments, a “mammal” includes humans, non-human primates, domestic and farm animals, and zoo, sports, or pet animals, such as dogs, horses, rabbits, cattle, pigs, hamsters, gerbils, mice, ferrets, rats, cats, monkeys, etc. In some embodiments, the subject is human.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

As used herein, a “composition” refers to any mixture of two or more products, substances, or compounds, including cells. It may be a solution, a suspension, liquid, powder, a paste, aqueous, non-aqueous or any combination thereof.

As used herein, the recitation of a numerical range for a variable is intended to convey that the invention may be practiced with the variable equal to any of the values within that range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values>0 and <2 if the variable is inherently continuous.

### IX. EXEMPLARY EMBODIMENTS

1. A genetically engineered T cell comprising:
(a) a first genetic disruption in the endogenous TRAC gene;
(b) a second genetic disruption in the endogenous B-2 microglobulin (B2M) gene;
(c) a nucleotide sequence comprising a transgene encoding a single chain HLA-E fusion protein; and
(d) a nucleotide sequence encoding a chimeric antigen receptor (CAR).

2. The genetically engineered T cell of embodiment 1, wherein the chimeric antigen receptor is directed against CD19.

3. The genetically engineered T cell of embodiment 2, further comprising a third genetic disruption.

4. The genetically engineered T cell of any of embodiments 1-3, wherein each genetic disruption is by a gene editing technique.

5. The genetically engineered T cell of embodiment 4, wherein the gene editing technique is or comprises a CRISPR-Cas system.

6. The genetically engineered T cell of embodiment 5, wherein the Cas is a Cas9.

7. The genetically engineered T cell of embodiment 6, wherein the Cas is a S. pyogenes Cas9 (spCas9).

8. The genetically engineered T cell of embodiment 5, wherein the Cas is a Cas12a.

9. The genetically engineered T cell of embodiment 8, wherein the Cas12 as is Francisella novicida Cas12a (FnCas12a), Lachnospiraceae bacterium Cas12a (LbCas12a), Acidaminococcus sp. Cas12a (AsCas12a).

10. The genetically engineered T cell of any of embodiments 1-9, wherein the first genetic disruption is by a CRISPR-Cas system that comprises a Cas protein and a guide RNA (gRNA) targeting the endogenous TRAC gene that comprises a spacer sequence that is complementary to a target site sequence in the endogenous TRAC gene, optionally wherein the Cas protein is a Cas9.

11. The genetically engineered T cell of any of embodiments 1-10, wherein the first genetic disruption in the endogenous TRAC gene is in a target site sequence in exon 1 of the TRAC gene.

12. The genetically engineered T cell of embodiment 11, wherein the target site sequence in exon 1 of the endogenous TRAC gene is located within a TRAC genome region at contiguous positions within the hg38 genomic region chr14:22,547,506-22,547,778

13. The genetically engineered T cell of embodiment 11 or embodiment 12, wherein the target site sequence in exon 1 of the endogenous TRAC gene is located at hg38 genomic coordinates chr14:22,547,576-22,547,595.

14. The genetically engineered T cell of any of embodiments 11-13, wherein the target site sequence in exon 1 of the endogenous TRAC gene has the sequence set forth in SEQ ID NO: 84, a contiguous portion thereof of at least 14 nucleotides (nt), or a complementary sequence of the foregoing.

15. The genetically engineered T cell of any of embodiments 11-14, wherein the target site sequence in exon 1 of the endogenous TRAC gene has the sequence set forth in SEQ ID NO: 84.

16. The genetically engineered T cell of any of embodiments 1-15, wherein the first genetic disruption is by a CRISPR-Cas system that comprises a Cas9 protein and a guide RNA (gRNA) comprising a spacer sequence comprising the nucleic acid sequence of SEQ ID NO: 87, or a contiguous portion thereof of at least 14 nt.

17. The genetically engineered T cell of any of embodiments 10-16, wherein the CRISPR-Cas system is a ribonucleoprotein (RNP) complex comprising the Cas9 protein and the gRNA.

18. The genetically engineered T cell of any of embodiments 1-17, wherein the first genetic disruption disrupts one or more alleles of the endogenous TRAC gene.

19. The genetically engineered T cell of any of embodiments 1-18, wherein the first genetic disruption disrupts all alleles of the endogenous TRAC gene.

20. The genetically engineered T cell of any of embodiments 1-19, wherein the first genetic disruption reduces protein expression of TCR alpha chain encoded from the endogenous TRAC gene, optionally protein expression of the TCR alpha chain on the surface of the T cell, more optionally wherein there is no detectable expression of TCR alpha chain in the T cell.

21. The genetically engineered T cell of any of embodiments 1-20, wherein the genetically engineered cell has reduced expression of CD3 on the cell surface, optionally wherein the genetically engineered cell does not express detectable CD3 on the cell surface.

22. The genetically engineered T cell of any of embodiments 1-21, wherein the second genetic disruption is by a CRISPR-Cas system that comprises a Cas protein and a guide RNA (gRNA) targeting the endogenous B2M gene that comprises a spacer sequence that is complementary to a target site sequence in the endogenous B2M gene, optionally wherein the Cas protein is a Cas12a.

23. The genetically engineered T cell of any of embodiments 1-22, wherein the second genetic disruption in the endogenous B2M gene is in a target site sequence in exon 2 of the B2M gene.

24. The genetically engineered T cell of embodiment 23, wherein the target site sequence in exon 2 of the endogenous B2M gene is located within a B2M genome region at contiguous positions within hg38 the genomic region 44,715,423-44,715,701.

25. The genetically engineered T cell of embodiment 23 or embodiment 24, wherein the target site sequence in exon 2 of the endogenous B2M gene is located at hg38 genomic coordinates chr15:44,715,614-44,715,634.

26. The genetically engineered T cell of any of embodiments 23-25, wherein the target site sequence in exon 2 of the endogenous B2M gene has the sequence set forth in SEQ ID NO: 85, a contiguous portion thereof of at least 14 nucleotides (nt), or a complementary sequence of the foregoing.

27. The genetically engineered T cell of any of embodiments 23-26, wherein the target site sequence has the sequence set forth in SEQ ID NO: 85.

28. The genetically engineered T cell of any of embodiments 1-25, wherein the second genetic disruption is by a CRISPR-Cas system that comprises a Cas12a protein and a guide RNA (gRNA) comprising a spacer sequence comprising the nucleic acid sequence SEQ ID NO: 105, or a contiguous portion thereof of at least 14 nt.

29. The genetically engineered T cell of any of embodiments 22-28, wherein the CRISPR-Cas system is a ribonucleoprotein (RNP) complex comprising the Cas12a protein and the gRNA.

30. The genetically engineered T cell of any of embodiments 1-29, wherein the second genetic disruption disrupts one or more alleles of the endogenous B2M gene.

31. The genetically engineered T cell of any of embodiments 1-30, wherein the second genetic disruption disrupts all alleles of the endogenous B2M gene.

32. The genetically engineered T cell of any of embodiments 1-31, wherein the second genetic disruption reduces protein expression of B2M encoded from the endogenous B2M gene, optionally wherein there is no detectable expression of endogenous B2M in the T cell.

33. The genetically engineered T cell of any of embodiments 1-32, wherein the genetically engineered cell has reduced expression of one or more HLA class I molecules (e.g., HLA-A class I, HLA-B class I and/or HLA-C class I) on the cell surface, optionally wherein the genetically engineered cell has no detectable expression of one or more HLA class I molecules (e.g., HLA-A class I, HLA-B class I and/or HLA-C class I) on the cell surface.

34. The genetically engineered T cell of any of embodiments 1-33, wherein the genetically engineered cell has no detectable expression of HLA-A class I, HLA-B class I and HLA-C class I on the cell surface.

35. The genetically engineered T cell of any of embodiments 10-34, wherein each gRNA independently comprises a spacer sequence between 14 nt and 24 nt, or between 16 nt and 22 nt in length.

36. The genetically engineered T cell of any of embodiments 10-35, wherein the gRNA independently comprises a spacer sequence that is 18 nt, 19 nt, 20 nt, 21 nt, or 22 nt in length.

37. The genetically engineered T cell of any of embodiments 10-36, wherein each gRNA further comprises a scaffold sequence for binding the respective Cas protein.

38. The genetically engineered T cell of embodiment 37, wherein the Cas protein is Cas9 or Cas12a.

39. The genetically engineered T cell of any of embodiments 10-38, wherein the gRNA is modified by one or more modified nucleotides, wherein the one or more modified nucleotides are for increased stability of the gRNA.

40. The genetically engineered T cells of any of embodiments 10-39, wherein the gRNA targeting the endogenous TRAC gene comprises the sequence set forth in SEQ ID NO: 82 or SEQ ID NO: 92.

41. The genetically engineered T cells of any of embodiments 22-40, wherein the gRNA targeting the endogenous B2M gene comprises the sequence set forth in SEQ ID NO: 83.

42. The genetically engineered T cell of any of embodiments 1-41, wherein the nucleotide sequence encoding the single chain HLA-E fusion protein is present in the disrupted B2M gene in the T cell under the operable control of a promoter.

43. The genetically engineered T cell of embodiment 42, wherein the promoter is the endogenous promoter of the B2M gene.

44. The genetically engineered T cell of embodiment 42, wherein the promoter is a heterologous promoter of the B2M gene.

45. The genetically engineered T cell of any of embodiments 42-44, wherein the nucleotide sequence has been integrated in the disrupted B2M gene by homology directed repair (HDR).

46. The genetically engineered T cell of any of embodiments 1-45, wherein the single chain HLA-E fusion protein comprises at least a portion of the B2M protein linked to at least a portion of an HLA-E class I chain.

47. The genetically engineered T cell of embodiment 46, wherein the at least a portion of the B2M protein is linked to at least a portion of an HLA-E class I chain by a peptide linker.

48. The genetically engineered T cell of embodiment 46 or embodiment 47, wherein the single chain HLA-E fusion protein further comprises a peptide linked to the fusion protein comprising at least a portion of the B2M and at least a portion of an HLA-E.

49. The genetically engineered T cell of embodiment 48, wherein the peptide is a peptide epitope that is presented by the single chain HLA-E fusion protein when expressed on the cell surface, optionally wherein presentation of the peptide on the cell surface ensures proper folding of the single chain fusion on the cell surface.

50. The genetically engineered T cell of any of embodiments 47-49, wherein the peptide is a portion of a signal sequence from an MHC class I molecule.

51. The genetically engineered T cell of embodiment 49 or embodiment 50, wherein the peptide is VMAPRTLVL (SEQ ID NO: 107), VMAPRTLLL (SEQ ID NO: 108), VMAPRTVLL (SEQ ID NO: 109), VMAPRTLFL (SEQ ID NO: 110), or VMAPRTLIL (SEQ ID NO: 111).

52. The genetically engineered T cell of any of embodiments 49-51, wherein the peptide is

| (SEQ ID NO: 107) | |
|---|---|
| | VMAPRTLVL |

53. The genetically engineered T cell of any of embodiments 49-52, wherein the peptide is linked to the fusion protein comprising at least a portion of the B2M protein and at least a portion of an HLA-E class I chain by a peptide linker.

54. The genetically engineered T cell of any of embodiments 47-53, wherein the peptide linker is a GS linker, optionally wherein the GS linker is 4 to 25 amino acids in length, optionally wherein the GS linker is 12 to 20 amino acids in length, more optionally at or about 15 amino acids in length.

55. The genetically engineered T cell of embodiment 54, wherein the GS linker is a (G4S)x3 linker is a GGGGSGGGGSGGGGS (SEQ ID NO: 112).

56. The genetically engineered T cell of any of embodiments 1-55, wherein the single chain HLA-E fusion protein comprises the sequence of amino acids set forth in SEQ ID NO: 81 or a sequence of amino acids that has at least about 85%, 86%, 87%, 88%, 89%, 90%, 91%, 92%, 93%, 94%, 95%, 96%, 97%, 98%, or 99% sequence identity to SEQ ID NO: 81.

57. The genetically engineered T cell of any of embodiments 1-56, wherein the single chain HLA-E fusion protein comprises the sequence of amino acids set forth in SEQ ID NO: 81

58. The genetically engineered T cell of any of embodiments 1-57, wherein the single chain fusion HLA-E fusion protein is capable of engaging inhibitory receptors on the surface of NK cells.

59. The genetically engineered T cell of any of embodiments 1-58, wherein the nucleotide sequence encoding the CAR is present in the disrupted TRAC gene in the T cell under the operable control of a promoter.

60. The genetically engineered T cell of embodiment 59, wherein the promoter is a heterologous promoter of the TRAC gene.

61. The genetically engineered T cell of embodiment 60, wherein the heterologous promoter is or comprises a human elongation factor 1 alpha (EF1α) promoter or a variant thereof.

62. The genetically engineered T cell of embodiment 59, wherein the promoter is the endogenous promoter of the TRAC gene.

63. The genetically engineered T cell of any of embodiments 59-62, wherein the nucleotide sequence has been integrated in the disrupted TRAC gene by homology directed repair (HDR).

64. The genetically engineered T cell of any of embodiments 1-63, wherein the CAR is directed against human or cynomolgus CD19.

65. The genetically engineered T cell of embodiment 64, wherein the CAR is directed against human CD19.

66. The genetically engineered T cell of any of embodiments 1-65, wherein the CAR comprises an extracellular domain, a spacer, a transmembrane domain, and an intracellular signaling domain.

67. The genetically engineered T cell of embodiment 66, wherein the extracellular domain comprises a CD19-binding domain comprising a V_{H} region and a V_{L} region.

68. The genetically engineered T cell of embodiment 67, wherein:
(i) the V_{H} region comprises CDR-H1, CDR-H2, and CDR-H3 sequences having the sequences of SEQ ID NOs: 3, 4, and 5, respectively; and the V_{L} region comprises CDR-L1, CDR-L2, and CDR-L3 sequences having the sequences of SEQ ID NOs: 7, 8, and 10, respectively;
(ii) the V_{H} region comprises CDR-H1, CDR-H2, and CDR-H3 sequences having the sequences of SEQ ID NOs: 3, 4, and 5, respectively; and the V_{L} region comprises CDR-L1, CDR-L2, and CDR-L3 sequences having the sequences of SEQ ID NOs: 7, 9, and 10, respectively;
(iii) the V_{H} region comprises CDR-H1, CDR-H2, and CDR-H3 sequences having the sequences of SEQ ID NOs: 3, 4, and 6, respectively; and the V_{L} region comprises CDR-L1, CDR-L2, and CDR-L3 sequences having the sequences of SEQ ID NOs: 7, 8, and 10, respectively; or
(iv) the V_{H} region comprises CDR-H1, CDR-H2, and CDR-H3 sequences having the sequences of SEQ ID NOs: 3, 4, and 6, respectively; and the V_{L} region comprises CDR-L1, CDR-L2, and CDR-L3 sequences having the sequences of SEQ ID NOs: 7, 9, and 10, respectively.

69. The genetically engineered T cell of embodiment 67 or 68, wherein the extracellular domain comprises in order from the amino- to carboxy-terminus:
(i) the V_{L} region of the CD19-binding domain and the V_{H} region of the CD19-binding domain; or
(ii) the V_{H} region of the CD19-binding domain and the V_{L} region of the CD19-binding domain.

70. The genetically engineered T cell of any of embodiments 67-69, wherein:
(i) the V_{H} region of the CD19-binding domain comprises the sequences set forth in, or a sequence that is at least 90%, 91%, 92%, 93%, 94%, 95%, 96%, 97%, 98% or 99% identical to, SEQ ID NO: 1; and
(ii) the V_{L} region of the CD19-binding domain comprises the sequences set forth in, or a sequence that is at least 90%, 91%, 92%, 93%, 94%, 95%, 96%, 97%, 98% or 99% identical to, SEQ ID NO: 2.

71. The genetically engineered T cell of any one of embodiments 67-70, wherein the V_{H} region of the CD19-binding domain comprises the sequences set forth in SEQ ID NO: 1; and the V_{L} region of the CD19-binding domain comprises the sequences set forth in SEQ ID NO: 2.

72. The genetically engineered T cell of any of embodiments 67-71, wherein the V_{H} region of the CD19-binding domain is joined to the V_{L} region of the CD19-binding domain via a linker.

73. The genetically engineered T cell of embodiment 72, wherein the linker is a flexible linker.

74. The genetically engineered T cell of embodiment 72 or 73, wherein the linker is 5 to 25 amino acids in length, optionally wherein the linker is 12 to 18 amino acids in length.

75. The genetically engineered T cell of any of embodiments 72-74, wherein the linker comprises the sequence set forth in SEQ ID NO: 18 or the sequence set forth in SEQ ID NO: 19.

76. The genetically engineered T cell of any of embodiments 66-75, wherein the spacer comprises a hinge region sequence, optionally wherein the hinge region sequence is a hinge region of an immunoglobulin or a variant thereof.

77. The genetically engineered T cell of embodiment 76, wherein the hinge region of an immunoglobulin is an IgG4 hinge region, optionally a human IgG4 hinge region, or a variant thereof.

78. The genetically engineered T cell of any one of embodiments 66-77, wherein the spacer comprises a variant IgG4 hinge region comprising substitution of amino acids CPSC to CPPC compared to the wild-type IgG4 hinge region.

79. The genetically engineered T cell of any one of embodiments 66-78, wherein the spacer is between 12 and 15 amino acids in length.

80. The genetically engineered T cell of any one of embodiments 66-79, wherein the spacer comprises an amino acid sequence having at least at or about 85%, 86%, 87%, 88%, 89%, 90%, 91%, 92%, 93%, 94%, 95%, 96%, 97%, 98% or 99% identity to SEQ ID NO: 12, optionally wherein the spacer has the sequence set forth in SEQ ID NO: 12.

81. The genetically engineered T cell of any one of embodiments 66-80, wherein the spacer is between 200 and 250 amino acids in length, or between 220 and 240 amino acids in length.

82. The genetically engineered T cell of any one of embodiments 66-78 and 81, wherein the spacer comprises a hinge region of an immunoglobulin, a CH2 region of an immunoglobulin or a chimeric CH2 region of two different immunoglobulins, and a CH3 region of an immunoglobulin.

83. The genetically engineered T cell of embodiment 82, wherein the spacer comprises an IgG4 hinge region or a variant thereof, a chimeric CH2 region comprising a portion of an IgG4 CH2 and a portion of an IgG2 CH2 (IgG2/4 CH2 region), and an IgG4 CH3 region.

84. The genetically engineered T cell of any one of embodiments 66-78 and 81-83, wherein the spacer comprises an amino acid sequence having at least at or about 85%, 86%, 87%, 88%, 89%, 90%, 91%, 92%, 93%, 94%, 95%, 96%, 97%, 98% or 99% identity to SEQ ID NO: 13, optionally wherein the spacer has the sequence set forth in SEQ ID NO: 13.

85. The genetically engineered T cell of any one of embodiments 66-84 wherein the transmembrane domain comprises a transmembrane domain from CD28, optionally a human CD28.

86. The genetically engineered T cell of any one of embodiments 66-85, wherein the transmembrane domain is or comprises SEQ ID NO: 15 or an amino acid sequence having at least at or about 90%, 91%, 92%, 93%, 94%, 95%, 96%, 97%, 98% or 99% sequence identity to SEQ ID NO: 15.

87. The genetically engineered T cell of any of embodiments 66-86, wherein the intracellular signaling domain is a cytoplasmic signaling domain of a CD3-zeta (CD3ζ) chain, optionally a human CD3ζ chain.

88. The genetically engineered T cell of any one of embodiments 66-87, wherein the intracellular signaling domain comprises the sequence set forth in SEQ ID NO: 17, or an amino acid sequence having at least at or about 90%, 91%, 92%, 93%, 94%, 95%, 96%, 97%, 98% or 99% sequence identity to SEQ ID NO: 17.

89. The genetically engineered T cell of any one of embodiments 66-88, wherein the intracellular signaling region further comprises a costimulatory signaling region.

90. The genetically engineered T cell of embodiment 89, wherein the costimulatory signaling region comprises an intracellular signaling domain of a T cell costimulatory molecule or a signaling portion thereof.

91. The genetically engineered T cell of embodiment 89 or embodiment 90, wherein the costimulatory signaling region comprises an intracellular signaling domain of 4-1BB, optionally a human 4-1BB.

92. The genetically engineered T cell of any of embodiments 66-91, wherein the costimulatory signaling region comprises the sequence set forth in SEQ ID NO: 16 or an amino acid sequence having at least at or about 90%, 91%, 92%, 93%, 94%, 95%, 96%, 97%, 98% or 99% sequence identity to SEQ ID NO: 16.

93. The genetically engineered T cell of any of embodiments 66-92, wherein the CAR comprises the amino acid sequence set forth in SEQ ID NO: 78 or SEQ ID NO: 138, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 78 or SEQ ID NO: 138.

94. The genetically engineered T cell of any of embodiments 1-93, wherein the genetically engineered T cell comprises one or more further genetic disruptions to reduce cell surface expression of one or more HLA class TT molecules.

95. The genetically engineered T cell of embodiment 94, wherein the one or more further genetic disruptions is a genetic disruption in the CIITA gene.

96. The genetically engineered T cell of any of embodiments 1-95, wherein the T cell is a primary T cell.

97. The genetically engineered T cell of embodiment 96, wherein the primary T cell is from a human donor.

98. The genetically engineered T cell of embodiment 97, wherein the human donor is a healthy donor aged 18 to 35 years old, a healthy donor having a body mass index (BMI) less than 30 kg/m², or a healthy donor aged 18 to 35 years old and having a body mass index (BMI) less than 30 kg/m².

99. A method of producing a genetically engineered T cell, the method comprising:
(a) introducing, into a T cell, a first agent for inducing a first genetic disruption at a target site sequence in an endogenous B-2 microglobulin (B2M) gene;
(b) introducing into the T cell a second agent for inducing a second genetic disruption at a target site sequence in an endogenous T cell receptor alpha constant (TRAC) gene;
(c) introducing into the T cell a polynucleotide comprising a transgene encoding a single chain HLA-E fusion protein; and
(e) introducing into the T cell a polynucleotide comprising a transgene encoding a chimeric antigen receptor (CAR).

100. The method of embodiment 99, wherein the CAR is directed against CD19.

101. The method of embodiment 100, wherein the method further comprises (e) introducing into the T cell a third agent for inducing a third genetic disruption at a target site within a endogenous CIITA gene.

102. The method of any of embodiments 99-101, wherein each genetic disruption is by a gene editing technique.

103. The method of embodiment 102, wherein each introduced agent mediates the gene editing technique and is or comprises a CRISPR-Cas system comprising a guide RNA (gRNA) comprising a spacer sequence that binds to the target site and a Cas protein.

104. The method of embodiment 103, wherein each CRISPR-Cas system is a ribonucleoprotein (RNP) complex comprising the Cas protein and the gRNA.

105. The method of any of embodiments 99-104, wherein the first agent is a first CRISPR-Cas system comprising a guide RNA (gRNA) targeting the endogenous TRAC gene comprising a spacer sequence that is complementary to the target site in the endogenous TRAC gene, and a Cas9 protein.

106. The method of embodiment 105, wherein the CRISPR-Cas system is a ribonucleoprotein (RNP) complex comprising the Cas9 protein and the gRNA.

107. The method of any of embodiments 103-106, wherein the Cas is a S. pyogenes Cas9 (spCas9).

108. The method of any of embodiments 99-107, wherein the target site sequence in the endogenous T cell receptor alpha constant (TRAC) gene is in exon 1 of the TRAC gene.

109. The method of any of embodiments 99-108, wherein the target site sequence in the endogenous TRAC gene is located within a TRAC genome region at contiguous positions within the hg38 genomic region chr14:22,547,506-22,547,778.

110. The method of any of embodiments 99-109, wherein the target site sequence in the endogenous TRAC gene is located at hg38 genomic coordinates chr14:22,547,576-22,547,595.

111. The method of any of embodiments 99-110, wherein the target site sequence in the endogenous TRAC gene has the sequence set forth in SEQ ID NO: 84, a contiguous portion thereof of at least 14 nucleotides (nt), or a complementary sequence of the foregoing.

112. The method of any of embodiments 99-111, wherein the target site sequence in the endogenous TRAC gene has the sequence set forth in SEQ ID NO: 84.

113. The method of any of embodiments 105-112, wherein the gRNA comprises a spacer sequence comprising the nucleic acid sequence of SEQ ID NO: 87, or a contiguous portion thereof of at least 14 nt.

114. The method of any of embodiments 99-113, wherein the first genetic disruption disrupts one or more alleles of the endogenous TRAC gene.

115. The method of any of embodiments 99-114, wherein the first genetic disruption disrupts all alleles of the endogenous TRAC gene.

116. The method of any of embodiments 99-115, wherein introducing the first agent into the T cell reduces protein expression of TCR alpha chain encoded from the endogenous TRAC gene, optionally protein expression of the TCR alpha chain on the surface of the T cell, more optionally wherein there is no detectable expression of TCR alpha chain in the T cell.

117. The method of any of embodiments 99-116, wherein introducing the first agent into the T cell reduces expression of CD3 on the cell surface, optionally where there is no detectable CD3 on the cell surface.

118. The method of any of embodiments 99-117, wherein the second agent is a second CRISPR-Cas system comprising a guide RNA (gRNA) targeting the endogenous B2M gene comprising a spacer sequence that is complementary to the target site in the endogenous B2M gene, and a Cas12a protein.

119. The method of embodiment 118, wherein the CRISPR-Cas system is a ribonucleoprotein (RNP) complex comprising the Cas12a protein and the gRNA.

120. The method of embodiment 103, embodiment 104, embodiment 118 or embodiment 119, wherein the Cas is a Cas12a is Francisella novicida Cas12a (FnCas12a), Lachnospiraceae bacterium Cas12a (LbCas12a), Acidaminococcus sp. Cas12a (AsCas12a).

121. The method of any of embodiments 99-120, wherein the target site sequence in the endogenous B2M gene is in exon 2 of the B2M gene.

122. The method of any of embodiments 99-121, wherein the target site sequence in the endogenous B2M gene is located within a B2M genome region at contiguous positions within hg38 the genomic region 44,715,423-44,715,701.

123. The method of any of embodiments 99-121, wherein the target site sequence in the endogenous B2M gene is located at hg38 genomic coordinates chr15:44,715,614-44,715,634.

124. The method of any of embodiments 99-123, wherein the target site sequence in the endogenous B2M gene has the sequence set forth in SEQ ID NO: 85, a contiguous portion thereof of at least 14 nucleotides (nt), or a complementary sequence of the foregoing.

125. The method of any of embodiments 99-124, wherein the target site sequence in the endogenous B2M gene has the sequence set forth in SEQ ID NO: 85.

126. The method of any of embodiments 118-125, wherein the gRNA comprises a spacer sequence comprising the nucleic acid sequence of SEQ ID NO: 105, or a contiguous portion thereof of at least 14 nt.

127. The method of any of embodiments 99-126, wherein the second genetic disruption disrupts one or more alleles of the endogenous B2M gene.

128. The method of any of embodiments 99-127, wherein the second genetic disruption disrupts all alleles of the endogenous B2M gene.

129. The method of any of embodiments 99-128, wherein introducing the second agent into the T cell reduces protein expression of B2M encoded from the endogenous B2M gene, optionally wherein there is no detectable expression of B2M in the T cell.

130. The method of any of embodiments 99-129, wherein introducing the second agent into the T cell reduces expression of one or more HLA class I molecules (e.g., HLA-A class I, HLA-B class I and/or HLA-C class I) on the cell surface, optionally wherein there is no detectable expression of one or more HLA class I molecules (e.g., HLA-A class I, HLA-B class I and/or HLA-C class I) on the cell surface.

131. The method of any of embodiments 99-130, wherein introducing the second agent into the T cell results in no detectable expression of HLA-A class I, HLA-B class I and HLA-C class I on the cell surface.

132. The method of any of embodiments 101-131, wherein the third agent is a third CRISPR-Cas system comprising a guide RNA (gRNA) targeting the endogenous CIITA gene comprising a spacer sequence that is complementary to the target site in the endogenous CIITA gene, and a Cas12a protein.

133. The method of embodiment 132, wherein the CRISPR-Cas system is a ribonucleoprotein (RNP) complex comprising the Cas12a protein and the gRNA.

134. The method of embodiments 103, embodiment 104, embodiment 132 or embodiment

133, wherein the Cas is a Cas12a is Francisella novicida Cas12a (FnCas12a), Lachnospiraceae bacterium Cas12a (LbCas12a), Acidaminococcus sp. Cas12a (AsCas12a).

135. The method of any of embodiments 103-134, wherein each gRNA independently comprises a spacer sequence between 14 nt and 24 nt, or between 16 nt and 22 nt in length.

136. The method of any of embodiments 103-135, wherein each gRNA independently comprises a spacer sequence that is 18 nt, 19 nt, 20 nt, 21 nt, or 22 nt in length.

137. The method of any of embodiments 103-136, wherein each gRNA further comprises a scaffold sequence for binding the respective Cas protein.

138. The method of any of embodiments 103-137, wherein the gRNA is modified by one or more modified nucleotides, wherein the one or more modified nucleotides are for increased stability of the gRNA.

139. The method of any of embodiments 105-138, wherein the gRNA targeting the endogenous TRAC gene comprises the sequence set forth in SEQ ID NO: 82 or SEQ ID NO: 92.

140. The method of any of embodiments 118-139, wherein the gRNA targeting the endogenous B2M gene comprises the sequence set forth in SEQ ID NO: 83.

141. The method of any of embodiments 103-140, wherein the gRNA targeting the endogenous TRAC gene and/or the gRNA targeting the endogenous B2M gene induces a double strand break.

142. The method of any of embodiments 99-141, wherein the transgene encoding a single chain HLA-E fusion protein is integrated via homology directed repair (HDR) at the target site in the B2M gene.

143. The method of any of embodiments 99-142, wherein the polynucleotide encoding the single chain HLA-E fusion protein further comprises one or more homology arm(s) linked to the transgene, wherein the one or more homology arm(s) comprise a sequence homologous to nucleic acid sequences surrounding the target site sequence in the endogenous B2M gene.

144. The method of embodiment 143, wherein the polynucleotide encoding the single chain HLA-E fusion protein comprises the structure [5′ homology arm]-[transgene]-[3′ homology arm], wherein the 5′ homology arm and 3′ homology arm comprises nucleic acid sequences homologous to the nucleic acid sequences surrounding the target site sequence in the endogenous B2M gene.

145. The method of embodiment 143 or embodiment 144, wherein the 5′ homology arm and 3′ homology arm independently are at or about 200, 300, 400, 500, 600, 700 or 800 nucleotides in length, or any value between any of the foregoing.

146. The method of any of embodiments 143-145, wherein the 5′ homology arm comprises SEQ ID NO: 79 or a sequence that has at least at or about 90%, 91%, 92%, 93%, 94%, 95%, 96%, 97%, 98% or 99% sequence identity to SEQ ID NO: 79 or a partial sequence thereof, and/or the 3′ homology arm comprises SEQ ID NO: 80, a sequence that has at least at or about 90%, 91%, 92%, 93%, 94%, 95%, 96%, 97%, 98% or 99% sequence identity to SEQ ID NO: 80 or a partial sequence thereof.

147. The method of any of embodiments 144-146, wherein the 5′ homology arm comprises SEQ ID NO: 79 and the 3′ homology arm comprises SEQ ID NO: 80.

148. The method of any of embodiments 99-147, wherein the single chain HLA-E fusion protein comprises at least a portion of the B2M protein linked to at least a portion of an HLA-E class I chain, optionally via a peptide linker.

149. The method of embodiment 148, wherein the single chain HLA-E fusion comprises a peptide sequence, wherein the peptide is a peptide epitope that is presented by the single chain HLA-E fusion protein when expressed on the cell surface.

150. The method of embodiment 149, wherein the peptide is VMAPRTLVL (SEQ ID NO: 107), VMAPRTLLL (SEQ ID NO: 108), VMAPRTVLL (SEQ ID NO: 109), VMAPRTLFL (SEQ ID NO: 110), or VMAPRTLIL (SEQ ID NO: 111), optionally wherein the peptide is VMAPRTLVL (SEQ ID NO: 107).

151. The method of any of embodiments 99-150, wherein the single chain HLA-E fusion protein comprises the sequence of amino acids set forth in SEQ ID NO: 81 or a sequence of amino acids that has at least about 85%, 86%, 87%, 88%, 89%, 90%, 91%, 92%, 93%, 94%, 95%, 96%, 97%, 98%, or 99% sequence identity to SEQ ID NO: 81.

152. The method of any of embodiments 99-151, wherein the transgene encoding the CAR is integrated via homology directed repair (HDR) at the target site in the TRAC gene.

153. The method of any of embodiments 99-152, wherein the polynucleotide encoding the CAR further comprises one or more homology arm(s) linked to the transgene, wherein the one or more homology arm(s) comprise a sequence homologous to nucleic acid sequences surrounding the target site sequence in the endogenous TRAC gene.

154. The method of embodiment 153, wherein the polynucleotide encoding the CAR comprises the structure [5′ homology arm]-[transgene]-[3′ homology arm], wherein the 5′ homology arm and 3′ homology arm comprises nucleic acid sequences homologous to the nucleic acid sequences surrounding the target site sequence in the endogenous TRAC gene.

155. The method of embodiment 153 or embodiment 154, wherein the 5′ homology arm and 3′ homology arm independently are at or about 200, 300, 400, 500, 600, 700 or 800 nucleotides in length, or any value between any of the foregoing.

156. The method of any of embodiments 153-155, wherein the 5′ homology arm comprises SEQ ID NO: 76 or a sequence that has at least at or about 90%, 91%, 92%, 93%, 94%, 95%, 96%, 97%, 98% or 99% sequence identity to SEQ ID NO: 76 or a partial sequence thereof, and/or the 3′ homology arm comprises SEQ ID NO: 77, a sequence that has at least at or about 90%, 91%, 92%, 93%, 94%, 95%, 96%, 97%, 98% or 99% sequence identity to SEQ ID NO: 77 or a partial sequence thereof.

157. The method of any of embodiments 153-156, wherein the 5′ homology arm comprises SEQ ID NO: 76 and the 3′ homology arm comprises SEQ ID NO: 77.

158. The method of any of embodiments 99-157, wherein the CAR is directed against human or cynomolgus CD19.

159. The method of embodiment 158, wherein the CAR is directed against human CD19.

160. The method of any of embodiments 99-159, wherein the CAR comprises an extracellular domain, a spacer, a transmembrane domain, and an intracellular signaling domain.

161. The method of embodiment 160, wherein the extracellular domain comprises a CD19-binding domain comprising a V_{H} region and a V_{L} region.

162. The method of embodiment 161, wherein
(i) the V_{H} region comprises CDR-H1, CDR-H2, and CDR-H3 sequences having the sequences of SEQ ID NOs: 3, 4, and 5, respectively; and the V_{L} region comprises CDR-L1, CDR-L2, and CDR-L3 sequences having the sequences of SEQ ID NOs: 7, 8, and 10, respectively;
(ii) the V_{H} region comprises CDR-H1, CDR-H2, and CDR-H3 sequences having the sequences of SEQ ID NOs: 3, 4, and 5, respectively; and the V_{L} region comprises CDR-L1, CDR-L2, and CDR-L3 sequences having the sequences of SEQ ID NOs: 7, 9, and 10, respectively;
(iii) the V_{H} region comprises CDR-H1, CDR-H2, and CDR-H3 sequences having the sequences of SEQ ID NOs: 3, 4, and 6, respectively; and the V_{L} region comprises CDR-L1, CDR-L2, and CDR-L3 sequences having the sequences of SEQ ID NOs: 7, 8, and 10, respectively; or
(iv) the V_{H} region comprises CDR-H1, CDR-H2, and CDR-H3 sequences having the sequences of SEQ ID NOs: 3, 4, and 6, respectively; and the V_{L} region comprises CDR-L1, CDR-L2, and CDR-L3 sequences having the sequences of SEQ ID NOs: 7, 9, and 10, respectively.

163. The method of embodiment 161 or embodiment 162, wherein the extracellular domain comprises in order from the amino- to carboxy-terminus:
(i) the V_{L} region of the CD19-binding domain and the V_{H} region of the CD19-binding domain; or
(ii) the V_{H} region of the CD19-binding domain and the V_{L} region of the CD19-binding domain.

164. The method of any of embodiments 161-163, wherein:
(i) the V_{H} region of the CD19-binding domain comprises the sequences set forth in, or a sequence that is at least 90%, 91%, 92%, 93%, 94%, 95%, 96%, 97%, 98% or 99% identical to, SEQ ID NO: 1; and
(ii) the V_{L} region of the CD19-binding domain comprises the sequences set forth in, or a sequence that is at least 90%, 91%, 92%, 93%, 94%, 95%, 96%, 97%, 98% or 99% identical to, SEQ ID NO: 2.

165. The method of any one of embodiments 161-164, wherein the V_{H} region of the CD19-binding domain comprises the sequences set forth in SEQ ID NO: 1; and the V_{L} region of the CD19-binding domain comprises the sequences set forth in SEQ ID NO: 2.

166. The method of any of embodiments 160-165, wherein the spacer comprises a hinge region sequence, optionally wherein the hinge region sequence is a hinge region of an immunoglobulin or a variant thereof.

167. The method of any one of embodiments 160-166 wherein the transmembrane domain comprises a transmembrane domain from CD28, optionally a human CD28.

168. The method of any of embodiments 160-167, wherein the intracellular signaling domain is a cytoplasmic signaling domain of a CD3-zeta (CD3ζ) chain, optionally a human CD3ζ chain.

169. The method of any one of embodiments 160-168, wherein the intracellular signaling region further comprises a costimulatory signaling region comprising an intracellular signaling domain of a T cell costimulatory molecule or a signaling portion thereof.

170. The method of embodiment 169, wherein the costimulatory signaling region comprises an intracellular signaling domain of 4-1BB, optionally a human 4-1BB.

171. The method of any of embodiments 99-170, wherein the CAR comprises the amino acid sequence set forth in SEQ ID NO: 78 or SEQ ID NO: 138, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 78 or SEQ ID NO: 138.

172. The method of any of embodiments 142-171, wherein the transgene encoding the single chain HLA-E fusion is integrated to be under the operable control of the endogenous B2M promoter, optionally wherein the transgene encoding the single chain HLA-E fusion protein comprises one or more multicistronic element(s) positioned upstream of the nucleotide sequence encoding the single chain HLA-E fusion, more optionally wherein the one or more multicistronic element is or comprises aT2A, a P2A, an E2A, or an F2A element.

173. The method of any of embodiments 153-172, wherein the transgene encoding the CAR is operably linked to a heterologous promoter to control expression of the CAR.

174. The method of embodiment 173, wherein the heterologous promoter is or comprises a human elongation factor 1 alpha (EF1α) promoter or a variant thereof.

175. The method of any of embodiments 99-174, wherein:
the introducing of the polynucleotide comprising a transgene encoding the single chain HLA-E fusion protein is by transduction of a first viral vector comprising the polynucleotide encoding the single chain HLA-E fusion; and/or
the introducing of the polynucleotide comprising a transgene encoding the CAR is by transduction of a second viral vector comprising the polynucleotide comprising a transgene encoding the CAR.

176. The method of embodiment 175, wherein a mixture comprising a first viral vector and a second viral vector are introduced into the T cell.

177. The method of embodiment 175 or embodiment 176, wherein the first viral vector and the second viral vector is an AAV vector, optionally wherein the AAV vector is an AAV6 vector.

178. The method of any of embodiments 99-177, wherein the first agent, the second agent and the third agent are introduced into the T cell via electroporation.

179. The method of embodiment 178, wherein each of the first agent, second agent and third agent are independently introduced as a ribonucleoprotein complex (RNP) and the total concentration of the RNPs introduced into the T cell is between at or about 1 μM and at or about 5 μM, between at or about 1.5 μM and at or about 2.5 μM, between at or about 1.7 μM and at or about 2.5 μM, or between at or about 2 μM and at or about 2.5 μM, optionally at or about 1.0 μM, at or about 1.5 μM, at or about 1.7 μM, at or about 2 μM, at or about 2.2 μM, or at or about 2.5 μM.

180. The method of embodiment 178, wherein each of the first agent and second agent are independently introduced as a ribonucleoprotein complex (RNP) and the total concentration of the RNPs introduced into the T cell is between at or about 1 μM and at or about 5 μM, between at or about 1.5 μM and at or about 2.5 μM, between at or about 1.7 μM and at or about 2.5 μM, or between at or about 2 μM and at or about 2.5 μM, optionally at or about 1.0 μM, at or about 1.5 μM, at or about 1.7 μM, at or about 2 μM, at or about 2.2 μM, or at or about 2.5 μM.

181. The method of any of embodiments 178-180, wherein the first agent is introduced as a RNP at a concentration of about 0.0625 μM to 0.5 μM RNP and the second agent is introduced as a RNP at a concentration of about 1.7 μM to 2.5 μM.

182. The method of embodiment 181, wherein the first agent is introduced as a RNP at a concentration of about 0.0625 μM to 0.5 μM RNP and the second agent is introduced as a RNP at a concentration of about 1.7 μM to 2.5 μM.

183. The method of embodiment 182, wherein the first agent is introduced as a RNP at a concentration of about 0.125 μM and the second agent is introduced as a RNP at a concentration of about 2 μM.

184. The method of any of embodiments 99-183, wherein the first agent and second agent are introduced simultaneously.

185. The method of any of embodiments 178-184, wherein after the electroporation, the method comprises introducing the polynucleotides by transducing the T cells with a mixture of viral vectors, wherein the mixture of viral vectors comprises a first viral vector comprising the polynucleotide encoding the single chain HLA-E fusion and a second viral vector comprising the polynucleotide comprising a transgene encoding the CAR.

186. The method of embodiment 185, wherein the first viral vector and the second viral vector is an AAV vector, optionally wherein the AAV vector is an AAV6 vector.

187. The method of embodiment 185 or embodiment 186, wherein transducing is within about 15 minutes, within about 30 minutes, within about 60 minutes, or within about 2 hours, after the introductions.

188. The method of any of embodiments 185-187, wherein after the transducing the method further comprises incubating the cell under static conditions in serum free media for a period of time for recovery of the cells.

189. The method of any of embodiments 185-188, wherein the method further comprises expanding the T cells 2 to 8 doublings or 6 to 7 doublings, optionally expanding the T cells in the presence of one or more recombinant cytokines, optionally in the presence of one or more recombinant IL-2, IL-7 and/or IL-15.

190. The method of embodiment 189, wherein the expanding is carried out with perfusion.

191. The method of any of embodiments 99-190, wherein prior to each of the introducing, the method comprises stimulating the T cells with one or more stimulatory agent(s) under conditions to stimulate or activate the T cells, optionally wherein the one or more stimulatory agent(s) comprises anti-CD3 and/or anti-CD28 antibodies, optionally anti-CD3/anti-CD28 Fabs.

192. The method of any of embodiments 99-191, wherein the T cell is a primary T cell.

193. The method of embodiment 192, wherein the primary T cell is from a human donor.

194. The method of embodiment 193, wherein the human donor is a healthy donor aged 18 to 35 years old, a healthy donor having a body mass index (BMI) less than 30 kg/m², or a healthy donor aged 18 to 35 years old and having a body mass index (BMI) less than 30 kg/m².

195. The method of any of embodiments 99-194, wherein the method is performed ex vivo.

196. The method of any of embodiments 99-195, wherein the method is performed in vitro.

197. The method of any of embodiments 99-196, further comprising harvesting T cells produced by the method.

198. The method of embodiment 197, further comprising depleting CD3+ T cells from the harvested T cells.

199. The method of embodiment 197 or embodiment 198, further comprising formulating the harvested T cells with a cryoprotectant.

200. A genetically engineered T cell produced by the method of any of embodiments 99-199.

201. A system for engineering a T cell, comprising:
(a) a first agent for inducing a first genetic disruption at a target site sequence in an endogenous endogenous B-2 microglobulin (B2M) gene;
(b) a second agent for inducing a second genetic disruption at a target site sequence in a endogenous T cell receptor alpha constant (TRAC) gene;
(c) a polynucleotide comprising a transgene encoding a single chain HLA-E fusion protein; and
(d) a polynucleotide comprising a transgene encoding a chimeric antigen receptor (CAR).

202. The system of embodiment 201, wherein the chimeric antigen receptor (CAR) is directed against CD19.

203. The system of embodiment 202, further comprising a third agent for inducing a third genetic disruption.

204. The system of embodiment 201-203, wherein the first agent, second agent and/or third agent is or comprises a CRISPR-Cas system comprising a guide RNA (gRNA) comprising a spacer sequence that binds to the target site and a Cas protein.

205. The system of embodiment 204, wherein each CRISPR-Cas system is a ribonucleoprotein (RNP) complex comprising the Cas protein and the gRNA.

206. The system of any of embodiments 201-205, wherein the first agent is a ribonucleoprotein complex comprising a guide RNA (gRNA) targeting the endogenous TRAC gene comprising a spacer sequence that is complementary to the target site in the endogenous TRAC gene, and a Cas9 protein, optionally wherein the Cas is a S. pyogenes Cas9 (spCas9).

207. The system of any of embodiments 201-206, wherein the target site sequence in the endogenous T cell receptor alpha constant (TRAC) gene is in exon 1 of the TRAC gene.

208. The system of any of embodiments 201-207, wherein the target site sequence in the endogenous TRAC gene has the sequence set forth in SEQ ID NO: 84, a contiguous portion thereof of at least 14 nucleotides (nt), or a complementary sequence of the foregoing.

209. The system of any of embodiments 204-208, wherein the gRNA comprises a spacer sequence comprising the nucleic acid sequence of SEQ ID NO: 87, or a contiguous portion thereof of at least 14 nt.

210. The system of any of embodiments 201-209, wherein the second agent is a ribonucleoprotein complex comprising a guide RNA (gRNA) targeting the endogenous B2M gene comprising a spacer sequence that is complementary to the target site in the endogenous B2M gene, and a Cas12a protein.

211. The system of any of embodiments 201-210, wherein the target site sequence in the endogenous B2M gene is in exon 2 of the B2M gene.

212. The system of any of embodiments 201-211, wherein the target site sequence in the endogenous B2M gene has the sequence set forth in SEQ ID NO: 85, a contiguous portion thereof of at least 14 nucleotides (nt), or a complementary sequence of the foregoing.

213. The system of any of embodiments 210-212, wherein the gRNA comprises a spacer sequence comprising the nucleic acid sequence of SEQ ID NO: 105, or a contiguous portion thereof of at least 14 nt.

214. The system of any of embodiments 201-213, wherein each gRNA further comprises a scaffold sequence for binding the respective Cas protein.

215. The system of any of embodiments 201-214, wherein the gRNA is modified by one or more modified nucleotides, wherein the one or more modified nucleotides are for increased stability of the gRNA.

216. The system of any of embodiments 201-215, wherein the gRNA targeting the endogenous TRAC gene comprises the sequence set forth in SEQ ID NO: 82 or SEQ ID NO: 92.

217. The system of any of embodiments 201-216, wherein the gRNA targeting the endogenous B2M gene comprises the sequence set forth in SEQ ID NO: 83.

218. The system of any of embodiments 201-217, wherein the polynucleotide encoding the single chain HLA-E fusion protein further comprises one or more homology arm(s) linked to the transgene, wherein the one or more homology arm(s) comprise a 5′ homology arm and a 3′ homology arm comprises nucleic acid sequences homologous to the nucleic acid sequences surrounding the target site sequence in the endogenous B2M gene.

219. The system of embodiment 218, wherein the 5′ homology arm comprises SEQ ID NO: 79 or a sequence that has at least at or about 90%, 91%, 92%, 93%, 94%, 95%, 96%, 97%, 98% or 99% sequence identity to SEQ ID NO: 79 or a partial sequence thereof, and/or the 3′ homology arm comprises SEQ ID NO: 80, a sequence that has at least at or about 90%, 91%, 92%, 93%, 94%, 95%, 96%, 97%, 98% or 99% sequence identity to SEQ ID NO: 80 or a partial sequence thereof.

220. The system of any of embodiments 201-219, wherein the single chain HLA-E fusion protein comprises the sequence of amino acids set forth in SEQ ID NO: 81 or a sequence of amino acids that has at least about 85%, 86%, 87%, 88%, 89%, 90%, 91%, 92%, 93%, 94%, 95%, 96%, 97%, 98%, or 99% sequence identity to SEQ ID NO: 81.

221. The system of any of embodiments 201-220, wherein the polynucleotide encoding the CAR further comprises one or more homology arm(s) linked to the transgene, wherein the one or more homology arm(s) comprise a 5′ homology arm and a 3′ homology arm comprising nucleic acid sequences homologous to the nucleic acid sequences surrounding the target site sequence in the endogenous TRAC gene.

222. The system of embodiment 221, wherein the 5′ homology arm comprises SEQ ID NO: 76 or a sequence that has at least at or about 90%, 91%, 92%, 93%, 94%, 95%, 96%, 97%, 98% or 99% sequence identity to SEQ ID NO: 76 or a partial sequence thereof, and/or the 3′ homology arm comprises SEQ ID NO: 77, a sequence that has at least at or about 90%, 91%, 92%, 93%, 94%, 95%, 96%, 97%, 98% or 99% sequence identity to SEQ ID NO: 77 or a partial sequence thereof.

223. The system of any of embodiments 201-222, wherein the CAR comprises an extracellular domain, a spacer, a transmembrane domain, and an intracellular signaling domain, wherein the extracellular domain comprises a CD19-binding domain that binds to CD19 comprising a V_{H} region and a V_{L} region.

224. The system of embodiment 223, wherein:
(i) the V_{H} region comprises CDR-H1, CDR-H2, and CDR-H3 sequences having the sequences of SEQ ID NOs: 3, 4, and 5, respectively; and the V_{L} region comprises CDR-L1, CDR-L2, and CDR-L3 sequences having the sequences of SEQ ID NOs: 7, 8, and 10, respectively;
(ii) the V_{H} region comprises CDR-H1, CDR-H2, and CDR-H3 sequences having the sequences of SEQ ID NOs: 3, 4, and 5, respectively; and the V_{L} region comprises CDR-L1, CDR-L2, and CDR-L3 sequences having the sequences of SEQ ID NOs: 7, 9, and 10, respectively;
(iii) the V_{H} region comprises CDR-H1, CDR-H2, and CDR-H3 sequences having the sequences of SEQ ID NOs: 3, 4, and 6, respectively; and the V_{L} region comprises CDR-L1, CDR-L2, and CDR-L3 sequences having the sequences of SEQ ID NOs: 7, 8, and 10, respectively;
(iv) the V_{H} region comprises CDR-H1, CDR-H2, and CDR-H3 sequences having the sequences of SEQ ID NOs: 3, 4, and 6, respectively; and the V_{L} region comprises CDR-L1, CDR-L2, and CDR-L3 sequences having the sequences of SEQ ID NOs: 7, 9, and 10, respectively; or
(v) the V_{H} region of the CD19-binding domain comprises the sequences set forth in, or a sequence that is at least 90%, 91%, 92%, 93%, 94%, 95%, 96%, 97%, 98% or 99% identical to, SEQ ID NO: 1; and the V_{L} region of the CD19-binding domain comprises the sequences set forth in, or a sequence that is at least 90%, 91%, 92%, 93%, 94%, 95%, 96%, 97%, 98% or 99% identical to, SEQ ID NO: 2.

225. The system of embodiment 223 or embodiment 224, wherein the extracellular domain comprises in order from the amino- to carboxy-terminus:
(i) the V_{L} region of the CD19-binding domain and the V_{H} region of the CD19-binding domain; or
(ii) the V_{H} region of the CD19-binding domain and the V_{L} region of the CD19-binding domain.

226. The system of any of embodiments 223-225, wherein the CAR comprises the amino acid sequence set forth in SEQ ID NO: 78 or SEQ ID NO: 138, or an amino acid sequence that is at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98% or at or about 99% sequence identity to the amino acid sequence set forth in SEQ ID NO: 78 or SEQ ID NO: 138.

227. The system of any of embodiments 201-226, wherein:
the polynucleotide comprising a transgene encoding the single chain HLA-E fusion protein is comprised in a first viral vector comprising the polynucleotide encoding the single chain HLA-E fusion; and/or
the polynucleotide comprising a transgene encoding the CAR is comprised in a second viral vector comprising the polynucleotide comprising a transgene encoding the CAR.

228. The system of embodiment 227, wherein the system comprises a mixture comprising a first viral vector and a second viral vector.

229. The system of embodiment 227 or embodiment 228, wherein the first viral vector and the second viral vector is an AAV vector, optionally wherein the AAV vector is an AAV6 vector.

230. A kit comprising the system of any of embodiments 201-229, and optionally instructions for using the system to genetically engineer a T cell.

231. A method of producing a genetically engineered T cell, the method comprising introducing the first agent, the second agent, the third agent, the polynucleotide comprising a transgene encoding an HLA-E fusion protein and the polynucleotide encoding the CAR of the system of any of embodiments 201-230 into a T cell.

232. A method of producing a genetically engineered T cell, the method comprising introducing the first agent, the second agent, the polynucleotide comprising a transgene encoding an HLA-E fusion protein and the polynucleotide encoding the CAR of the system of any of embodiments 201-230 into a T cell.

233. A composition comprising a population of genetically engineered T cells of any of embodiments 1-98.

234. A composition comprising a population of genetically engineered T cells produced by the method of any of embodiments 99-199.

235. The composition of embodiment 233 or embodiment 234, wherein the composition is a pharmaceutical composition comprising a pharmaceutically acceptable excipient.

236. The composition of any of embodiments 233-235, wherein the composition comprises a cyroprotectant, optionally wherein the cryoprotectant is DMSO.

237. The composition of any of embodiments 233-236, wherein at least at or about 70%, 75%, 80%, 85%, 90%, or 95% of the engineered T cells, or of the total cells or total T cells, in the composition comprise a genetic disruption in the endogenous TRAC gene.

238. The composition of any of embodiments 233-237, wherein at least at or about 70%, 75%, 80%, 85%, 90%, or 95% of the engineered T cells, or of the total cells or total T cells, in the composition comprise no detectable expression of TCR alpha chain in the T cell.

239. The composition of any of embodiments 233-238, wherein at least at or about 70%, 75%, 80%, 85%, 90%, or 95% of the engineered T cells, or of the total cells or total T cells, in the composition comprise no detectable expression of CD3 on the cell surface of the T cell.

240. The composition of any of embodiments 233-239, wherein at least at or about 95%, 96%, 97% or 98% of the engineered T cells, or of the total cells or total T cells, in the composition comprise no detectable expression of CD3 on the cell surface of the T cell.

241. The composition of any of embodiments 233-240, wherein at least at or about 70%, 75%, 80%, 85%, 90%, or 95% of the engineered T cells, or of the total cells or total T cells, in the composition comprise a genetic disruption in the endogenous B2M gene.

242. The composition of any of embodiments 233-241, wherein at least at or about 70%, 75%, 80%, 85%, 90%, or 95% of the engineered T cells, or of the total cells or total T cells, in the composition comprise no detectable expression of B2M in the T cell.

243. The composition of any of embodiments 233-242, wherein at least at or about 70%, 75%, 80%, 85%, 90%, or 95% of the engineered T cells, or of the total cells or total T cells, in the composition comprise no detectable expression of one or more HLA class I molecules (e.g., HLA-A class I, HLA-B class I and/or HLA-C class I) on the cell surface.

244. The composition of any of embodiments 233-243, wherein at least at or about 70%, 75%, 80%, 85%, 90%, or 95% of the engineered T cells, or of the total cells or total T cells, in the composition comprise no detectable expression of HLA-A class I, HLA-B class I and HLA-C class I on the cell surface.

245. The composition of any of embodiments 233-244, wherein at least at or about 50%, 60%, 65%, 70%, 75%, 80%, 85%, 90%, or 95% of the engineered T cells, or of the total cells or total T cells, in the composition express the single chain HLA-E fusion.

246. The composition of any of embodiments 233-245, wherein at least at or about 50%, 60%, 65%, 70%, 75%, 80%, 85%, 90%, or 95% of the engineered T cells, or of the total cells or total T cells, in the composition express the CAR.

247. The composition of any of embodiments 233-246, wherein at least at or about 50%, 60%, 65%, 70%, 75%, 80%, 85%, 90%, or 95% of the engineered T cells, or of the total cells or total T cells, in the composition express the single chain HLA-E fusion and the CAR.

248. The composition of any of embodiments 233-247, wherein at least 90% of the total cells in the composition comprise a genetic disruption in the endogenous TRAC gene, at least 90% of the total cells in the composition comprise a genetic disruption in the endogenous B2M gene, at least 50% of the total cells in the composition express the HLA-E fusion and at least 50% of the total cells in the composition express the CAR.

249. The composition of any of embodiments 233-247, wherein at least at or about 95%, 96%, 97% or 98% of the total cells in the composition comprise no detectable expression of CD3 on the cell surface of the T cell.

250. The composition of any of embodiments 233-249, wherein at least at or about 95%, 96%, 97% or 98% of the total cells in the composition comprise no detectable expression of HLA-A class I, HLA-B class I and HLA-C class I on the cell surface.

251. The composition of any of embodiments 233-250, wherein at least at or about 90%, 91%, 92%, 93%, 94%, 95%, 96%, 97%, 98%, or 99% of the cells in the composition are T cells.

252. The composition of any of embodiments 233-251, wherein the composition comprises CD4+ T cells and CD8+ T cells.

253. The composition of embodiment 252, wherein the ratio of CD4+ T cells to CD8+ T cells is from at or about 1:5 to at or about 5:1, optionally from at or about 1:3 to at or about 3:1.

254. The composition of any of embodiments 233-253, wherein at least at or about 50%, 55%, 60%, 65%, 70%, 75%, 80%, 85%, 90%, or 95% of the engineered T cells, or of the total cells or total T cells, in the composition are viable cells.

255. The composition of any of embodiments 233-254, wherein at least at or about 90%, 91%, 92%, 93%, 94%, 95%, 96%, 97%, 98%, or 99% of the cells in the composition are engineered T cells comprising a genetic disruption of one or more endogenous genes and expression of one or more transgene.

256. The composition of any of embodiments 233-255, wherein the population of T cells are from a healthy donor aged 18 to 35 years old and having a body mass index (BMI) less than 30 kg/m², wherein the T cells comprise (i) a modified TRAC locus comprising a transgene sequence encoding a CD19 CAR having an amino acid sequence set forth in SEQ ID NO: 138, and (ii) a modified B2M locus comprising a transgene sequence comprising the sequence set forth in SEQ ID NO: 132.

257. The composition of any of embodiments 233-255, wherein the population of T cells from a healthy donor aged 18 to 35 years old and having a body mass index (BMI) less than 30 kg/m², wherein the T cells comprise (i) a modified TRAC locus comprising a transgene sequence encoding a CD19 CAR having an amino acid sequence set forth in SEQ ID NO: 78, and (ii) a modified B2M locus comprising a transgene sequence comprising the sequence set forth in SEQ ID NO: 81.

258. The composition of any of embodiments 233-257, wherein the composition has one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, twenty-four or more, twenty-five or more, twenty-six or more, twenty-seven or more, or all the following attributes:
(i) at least about 50%, at least about 55%, at least about 60%, at least about 65%, at least about 70%, at least about 75%, at least about 80%, at least about 85%, at least about 90%, or at least about 95%, at least about 96%, at least about 97%, at least about 98%, at least about 99%, or 100% of the T cells are viable;
(ii) at least about 70%, at least about 75%, at least about 80%, at least about 85%, at least about 90%, at least about 95%, at least about 96%, at least about 97%, at least about 98%, at least about 99%, or 100% of the T cells are CD2+CD5+;
(iii) at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, at least about 96%, at least about 97%, at least about 98%, at least about 99%, or 100% of the T cells express CD19 CAR, such as the CD19 CAR having an amino acid sequence set forth in SEQ ID NO: 78 or SEQ ID NO: 138;
(iv) less than about 1%, less than about 2%, less than about 3%, less than about 4%, less than about 5%, less than about 6%, less than about 7%, less than about 8%, less than about 9%, or less than about 10% of the T cells are HLA-I+;
(v) the ratio of % TCR+ to % CAR+ T cells is less than about 0.005, less than about 0.006, less than about 0.007, less than about 0.008, less than about 0.009, or less than about 0.01;
(vi) less than about 1% of total alleles, less than about 2% of total alleles, less than about 3% of total alleles, less than about 4% of total alleles, less than about 5% of total alleles, less than about 6% of total alleles, less than about 7% of total alleles, less than about 8% of total alleles, less than about 9% of total alleles, or less than about 10% of total alleles have translocation between TRAC and B2M;
(vii) at least about 5,000,000, at least about 5,500,000, at least about 6,000,000, at least about 6,500,000, at least about 7,000,000, at least about 7,500,000, at least about 8,000,000, at least about 8,500,000, at least about 9,000,000, at least about 9,500,000, at least about 10,000,000, at least about 11,000,000, at least about 12,000,000, at least about 13,000,000, at least about 14,000,000, at least about 15,000,000, at least about 20,000,000, at least about 25,000,000, at least about 30,000,000, at least about 35,000,000, at least about 40,000,000, at least about 45,000,000, or at least about 50,000,000 viable CD2+CD5+CD19 CAR+ cells per mL of the composition;
(viii) the T cells are from a single healthy donor aged 18 to 35 years old;
(ix) the T cells are from a single healthy donor having a body mass index (BMI) less than 30 kg/m²;
(x) at least about 5% of total alleles, at least about 10% of total alleles, at least about 15% of total alleles, at least about 20% of total alleles, at least about 25% of total alleles, at least about 30% of total alleles, at least about 35% of total alleles, at least about 40% of total alleles, at least about 45% of total alleles, at least about 50% of total alleles, at least about 55% of total alleles, at least about 60% of total alleles, at least about 65% of total alleles, at least about 70% of total alleles, at least about 75% of total alleles, at least about 80% of total alleles, at least about 85% of total alleles, at least about 90% of total alleles, at least about 95% of total alleles, at least about 96% of total alleles, at least about 97% of total alleles, at least about 98% of total alleles, at least about 99% of total alleles, or 100% of total alleles have edited TRAC loci;
(xi) less than about 0.1%, less than about 0.2%, less than about 0.3%, less than about 0.4%, less than about 0.5%, less than about 0.6%, less than about 0.7%, less than about 0.8%, less than about 0.9%, or less than about 1% of the T cells are TCR+ T cells;
(xii) at least about 5% of total alleles, at least about 10% of total alleles, at least about 15% of total alleles, at least about 20% of total alleles, at least about 25% of total alleles, at least about 30% of total alleles, at least about 35% of total alleles, at least about 40% of total alleles, at least about 45% of total alleles, at least about 50% of total alleles, at least about 55% of total alleles, at least about 60% of total alleles, at least about 65% of total alleles, at least about 70% of total alleles, at least about 75% of total alleles, at least about 80% of total alleles, at least about 85% of total alleles, at least about 90% of total alleles, at least about 95% of total alleles, at least about 96% of total alleles, at least about 97% of total alleles, at least about 98% of total alleles, at least about 99% of total alleles, or 100% of total alleles have edited B2M loci;
(xiii) at least about 5% of total alleles, at least about 10% of total alleles, at least about 15% of total alleles, at least about 20% of total alleles, at least about 25% of total alleles, at least about 30% of total alleles, at least about 35% of total alleles, at least about 40% of total alleles, at least about 45% of total alleles, at least about 50% of total alleles, at least about 55% of total alleles, at least about 60% of total alleles, at least about 65% of total alleles, at least about 70% of total alleles, at least about 75% of total alleles, at least about 80% of total alleles, at least about 85% of total alleles, at least about 90% of total alleles, at least about 95% of total alleles, at least about 96% of total alleles, at least about 97% of total alleles, at least about 98% of total alleles, at least about 99% of total alleles, or 100% of total alleles have a nucleic acid encoding HLA-E(A) knocked-in at the B2M locus, such as a nucleic acid comprising the sequence set forth in SEQ ID NO: 86, or a nucleic acid encoding HLA-E(A) having an amino acid sequence set forth in SEQ ID NO: 81 or SEQ ID NO: 132;
(xiv) at least about 5% of total alleles, at least about 10% of total alleles, at least about 15% of total alleles, at least about 20% of total alleles, at least about 25% of total alleles, at least about 30% of total alleles, at least about 35% of total alleles, at least about 40% of total alleles, at least about 45% of total alleles, at least about 50% of total alleles, at least about 55% of total alleles, at least about 60% of total alleles, at least about 65% of total alleles, at least about 70% of total alleles, at least about 75% of total alleles, at least about 80% of total alleles, at least about 85% of total alleles, at least about 90% of total alleles, at least about 95% of total alleles, at least about 96% of total alleles, at least about 97% of total alleles, at least about 98% of total alleles, at least about 99% of total alleles, or 100% of total alleles have a nucleic acid encoding CD19 CAR knocked-in at the TRAC locus, such as a nucleic acid comprising the sequence set forth in SEQ ID NO: 136, or a nucleic acid encoding a CD19 CAR having an amino acid sequence set forth in SEQ ID NO: 78 or SEQ ID NO: 138;
(xv) less than about 1 ng, less than about 2 ng, less than about 3 ng, less than about 4 ng, less than about 5 ng, less than about 6 ng, less than about 7 ng, less than about 8 ng, less than about 9 ng, less than about 10 ng, less than about 11 ng, less than about 12 ng, less than about 13 ng, less than about 14 ng, less than about 15 ng, less than about 16 ng, less than about 17 ng, less than about 18 ng, less than about 19 ng, or less than about 20 ng of Cas9 or Cas12a per mL of the composition;
(xvi) less than about 0.01 fg, less than about 0.02 fg, less than about 0.03 fg, less than about 0.04 fg, less than about 0.05 fg, less than about 0.06 fg, less than about 0.07 fg, less than about 0.08 fg, less than about 0.09 fg, or less than about 1.0 fg of Cas9 or Cas12a per T cell;
(xvii) less than about 1×10¹⁰, less than about 5×10¹⁰, less than about 1×10¹¹, less than about 5×10¹¹, or less than about 1×10¹² AAV6 capsids per mL of the composition;
(xviii) ten or fewer, nine or fewer, eight or fewer, seven or fewer, six or fewer, five or fewer, four or fewer, three or fewer, two or fewer, one or fewer, or no verified off-target indel sites at a genomic locus other than TRAC and B2M;
(xix) less than about 0.5% of total alleles, less than about 1.0% of total alleles, less than about 1.5% of total alleles, less than about 2.0% of total alleles, less than about 2.5% of total alleles, less than about 3.0% of total alleles, less than about 3.5% of total alleles, or less than about 4.0% of total alleles have AAV6 integration at TRAC locus;
(xx) less than about 0.5% of total alleles, less than about 0.6% of total alleles, less than about 0.7% of total alleles, less than about 0.8% of total alleles, less than about 0.9% of total alleles, less than about 1.0% of total alleles, less than about 1.1% of total alleles, less than about 1.2% of total alleles, less than about 1.3% of total alleles, less than about 1.4% of total alleles, less than about 1.5% of total alleles, less than about 1.6% of total alleles, less than about 1.7% of total alleles, less than about 1.8% of total alleles, less than about 1.9% of total alleles, or less than about 2.0% of total alleles have AAV6 integration at B2M locus;
(xxi) undetectable AAV6 integration at loci other than TRAC and B2M;
(xxii) less than about 100 ng, less than about 150 ng, less than about 200 ng, less than about 250 ng, less than about 300 ng, less than about 350 ng, less than about 400 ng, less than about 450 ng, less than about 500 ng, less than about 550 ng, less than about 600 ng, less than about 650 ng, less than about 700 ng, less than about 750 ng, less than about 800 ng, less than about 850 ng, less than about 900 ng, less than about 950 ng, less than about 1000 ng, less than about 1100 ng, less than about 1200 ng, less than about 1300 ng, less than about 1400 ng, or less than about 1500 ng of Strep-Tactin Multimer activation reagent per mL of the composition;
(xxiii) no detected bacterial growth;
(xxiv) no more than about 1 EU/mL, no more than about 2 EU/mL, no more than about 3 EU/mL, no more than about 4 EU/mL, no more than about 5 EU/mL, no more than about 6 EU/mL, no more than about 7 EU/mL, no more than about 8 EU/mL, no more than about 9 EU/mL, no more than about 10 EU/mL, no more than about 11 EU/mL, no more than about 12 EU/mL, no more than about 13 EU/mL, no more than about 14 EU/mL, or no more than about 15 EU/mL endotoxin;
(xxv) no detected mycoplasma
(xxvi) no cytokine-independent growth;
(xxvii) no significant unexpected karyotype; or
(xxviii) negative for the presence of HIV, HTLV, HAV, HBV, HCV, CMV, EBV, HHV6, HHV7, HHV8, and B19.

259. The composition of any one of embodiments 233-258, wherein the composition has one or more, two or more, three or more, four or more, five or more, six or more, seven or more, eight or more, nine or more, ten or more, eleven or more, twelve or more, thirteen or more, fourteen or more, or all the following attributes:
(i) at least about 50%, at least about 55%, at least about 60%, at least about 65%, at least about 70%, at least about 75%, at least about 80%, at least about 85%, at least about 90%, or at least about 95%, at least about 96%, at least about 97%, at least about 98%, at least about 99%, or 100% of the T cells are viable;
(ii) at least about 70%, at least about 75%, at least about 80%, at least about 85%, at least about 90%, at least about 95%, at least about 96%, at least about 97%, at least about 98%, at least about 99%, or 100% of the T cells are CD2+CD5+;
(iii) at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, at least about 96%, at least about 97%, at least about 98%, at least about 99%, or 100% of the T cells express CD19 CAR, such as the CD19 CAR having an amino acid sequence set forth in SEQ ID NO: 78 or SEQ ID NO: 138;
(iv) less than about 1%, less than about 2%, less than about 3%, less than about 4%, less than about 5%, less than about 6%, less than about 7%, less than about 8%, less than about 9%, or less than about 10% of the T cells are HLA-I+;
(v) the ratio of % TCR+ to % CAR+ T cells is less than about 0.005, less than about 0.006, less than about 0.007, less than about 0.008, less than about 0.009, or less than about 0.01;
(vi) less than about 1% of total alleles, less than about 2% of total alleles, less than about 3% of total alleles, less than about 4% of total alleles, less than about 5% of total alleles, less than about 6% of total alleles, less than about 7% of total alleles, less than about 8% of total alleles, less than about 9% of total alleles, or less than about 10% of total alleles have translocation between TRAC and B2M;
(vii) at least about 5,000,000, at least about 5,500,000, at least about 6,000,000, at least about 6,500,000, at least about 7,000,000, at least about 7,500,000, at least about 8,000,000, at least about 8,500,000, at least about 9,000,000, at least about 9,500,000, at least about 10,000,000, at least about 11,000,000, at least about 12,000,000, at least about 13,000,000, at least about 14,000,000, at least about 15,000,000, at least about 20,000,000, at least about 25,000,000, at least about 30,000,000, at least about 35,000,000, at least about 40,000,000, at least about 45,000,000, or at least about 50,000,000 viable CD2+CD5+CD19 CAR+ cells per mL of the composition;
(viii) no detected bacterial growth;
(ix) no more than about 1 EU/mL, no more than about 2 EU/mL, no more than about 3 EU/mL, no more than about 4 EU/mL, no more than about 5 EU/mL, no more than about 6 EU/mL, no more than about 7 EU/mL, no more than about 8 EU/mL, no more than about 9 EU/mL, no more than about 10 EU/mL, no more than about 11 EU/mL, no more than about 12 EU/mL, no more than about 13 EU/mL, no more than about 14 EU/mL, or no more than about 15 EU/mL endotoxin;
(x) no detected mycoplasma;
(xi) no cytokine-independent growth;
(xii) no significant unexpected karyotype;
(xiii) negative for the presence of HIV, HTLV, HAV, HBV, HCV, CMV, EBV, HHV6, HHV7, HHV8, and B19.
(xiv) the T cells are from a single healthy donor aged 18 to 35 years old; or
(xv) the T cells are from a single healthy donor having a body mass index (BMI) less than 30 kg/n².

260. The composition of any of embodiments 233-259, wherein the composition comprises a population of T cells wherein the T cells are from a single healthy donor aged 18 to 35 years old and having a body mass index (BMI) less than 30 kg/m², and wherein the composition has one or more, two or more, three or more, four or more, five or more, six or more, seven or more, eight or more, nine or more, ten or more, eleven or more, twelve or more, or all the following attributes:
(i) at least about 50%, at least about 55%, at least about 60%, at least about 65%, at least about 70%, at least about 75%, at least about 80%, at least about 85%, at least about 90%, or at least about 95%, at least about 96%, at least about 97%, at least about 98%, at least about 99%, or 100% of the T cells are viable;
(ii) at least about 70%, at least about 75%, at least about 80%, at least about 85%, at least about 90%, at least about 95%, at least about 96%, at least about 97%, at least about 98%, at least about 99%, or 100% of the T cells are CD2+CD5+;
(iii) at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, at least about 96%, at least about 97%, at least about 98%, at least about 99%, or 100% of the T cells express CD19 CAR, such as the CD19 CAR having an amino acid sequence set forth in SEQ ID NO: 78 or SEQ ID NO: 138;
(iv) less than about 1%, less than about 2%, less than about 3%, less than about 4%, less than about 5%, less than about 6%, less than about 7%, less than about 8%, less than about 9%, or less than about 10% of the T cells are HLA-I+;
(v) the ratio of % TCR+ to % CAR+ T cells is less than about 0.005, less than about 0.006, less than about 0.007, less than about 0.008, less than about 0.009, or less than about 0.01;
(vi) less than about 1% of total alleles, less than about 2% of total alleles, less than about 3% of total alleles, less than about 4% of total alleles, less than about 5% of total alleles, less than about 6% of total alleles, less than about 7% of total alleles, less than about 8% of total alleles, less than about 9% of total alleles, or less than about 10% of total alleles have translocation between TRAC and B2M;
(vii) at least about 5,000,000, at least about 5,500,000, at least about 6,000,000, at least about 6,500,000, at least about 7,000,000, at least about 7,500,000, at least about 8,000,000, at least about 8,500,000, at least about 9,000,000, at least about 9,500,000, at least about 10,000,000, at least about 11,000,000, at least about 12,000,000, at least about 13,000,000, at least about 14,000,000, at least about 15,000,000, at least about 20,000,000, at least about 25,000,000, at least about 30,000,000, at least about 35,000,000, at least about 40,000,000, at least about 45,000,000, or at least about 50,000,000 viable CD2+CD5+CD19 CAR+ cells per mL of the composition;
(viii) no detected bacterial growth;
(ix) no more than about 1 EU/mL, no more than about 2 EU/mL, no more than about 3 EU/mL, no more than about 4 EU/mL, no more than about 5 EU/mL, no more than about 6 EU/mL, no more than about 7 EU/mL, no more than about 8 EU/mL, no more than about 9 EU/mL, no more than about 10 EU/mL, no more than about 11 EU/mL, no more than about 12 EU/mL, no more than about 13 EU/mL, no more than about 14 EU/mL, or no more than about 15 EU/mL endotoxin;
(x) no detected mycoplasma;
(xi) no cytokine-independent growth;
(xii) no significant unexpected karyotype; or
(xiii) negative for the presence of HIV, HTLV, HAV, HBV, HCV, CMV, EBV, HHV6, HHV7, HHV8, and B19.

261. The composition of any of embodiments 233-260, wherein the composition comprises a population of T cells wherein the T cells are from a single healthy donor aged 18 to 35 years old and having a body mass index (BMI) less than 30 kg/m², and wherein the composition has one or more, two or more, three or more, four or more, five or more, six or more, seven or more, eight or more, nine or more, ten or more, eleven or more, twelve or more, or all the following attributes:
(i) at least about 50%, at least about 55%, at least about 60%, at least about 65%, at least about 70%, at least about 75%, at least about 80%, at least about 85%, at least about 90%, or at least about 95%, at least about 96%, at least about 97%, at least about 98%, at least about 99%, or 100% of the T cells are viable;
(ii) at least about 70%, at least about 75%, at least about 80%, at least about 85%, at least about 90%, at least about 95%, at least about 96%, at least about 97%, at least about 98%, at least about 99%, or 100% of the T cells are CD2+CD5+;
(iii) at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, at least about 96%, at least about 97%, at least about 98%, at least about 99%, or 100% of the T cells express CD19 CAR, such as the CD19 CAR having an amino acid sequence set forth in SEQ ID NO: 78 or SEQ ID NO: 138;
(iv) less than about 1%, less than about 2%, less than about 3%, less than about 4%, less than about 5%, less than about 6%, less than about 7%, less than about 8%, less than about 9%, or less than about 10% of the T cells are HLA-I+;
(v) the ratio of % TCR+ to % CAR+ T cells is less than about 0.005, less than about 0.006, less than about 0.007, less than about 0.008, less than about 0.009, or less than about 0.01;
(vi) less than about 1% of total alleles, less than about 2% of total alleles, less than about 3% of total alleles, less than about 4% of total alleles, less than about 5% of total alleles, less than about 6% of total alleles, less than about 7% of total alleles, less than about 8% of total alleles, less than about 9% of total alleles, or less than about 10% of total alleles have translocation between TRAC and B2M;
(vii) at least about 5,000,000, at least about 5,000,000, at least about 5,500,000, at least about 6,000,000, at least about 6,500,000, at least about 7,000,000, at least about 7,500,000, at least about 8,000,000, at least about 8,500,000, at least about 9,000,000, at least about 9,500,000, at least about 10,000,000, at least about 11,000,000, at least about 12,000,000, at least about 13,000,000, at least about 14,000,000, at least about 15,000,000, at least about 20,000,000, at least about 25,000,000, at least about 30,000,000, at least about 35,000,000, at least about 40,000,000, at least about 45,000,000, or at least about 50,000,000 viable CD2+CD5+CD19 CAR+ cells per mL of the composition;
(viii) no detected bacterial growth;
(ix) no more than about 1 EU/mL, no more than about 2 EU/mL, no more than about 3 EU/mL, no more than about 4 EU/mL, no more than about 5 EU/mL, no more than about 6 EU/mL, no more than about 7 EU/mL, no more than about 8 EU/mL, no more than about 9 EU/mL, no more than about 10 EU/mL, no more than about 11 EU/mL, no more than about 12 EU/mL, no more than about 13 EU/mL, no more than about 14 EU/mL, or no more than about 15 EU/mL endotoxin;
(x) no detected mycoplasma;
(xi) no cytokine-independent growth;
(xii) no significant unexpected karyotype; or
(xiii) negative for the presence of HIV, HTLV, HAV, HBV, HCV, CMV, EBV, HHV6, HHV7, HHV8, and B19, and wherein the composition has one or more of the following attributes:
(a) at least about 5% of total alleles, at least about 10% of total alleles, at least about 15% of total alleles, at least about 20% of total alleles, at least about 25% of total alleles, at least about 30% of total alleles, at least about 35% of total alleles, at least about 40% of total alleles, at least about 45% of total alleles, at least about 50% of total alleles, at least about 55% of total alleles, at least about 60% of total alleles, at least about 65% of total alleles, at least about 70% of total alleles, at least about 75% of total alleles, at least about 80% of total alleles, at least about 85% of total alleles, at least about 90% of total alleles, at least about 95% of total alleles, at least about 96% of total alleles, at least about 97% of total alleles, at least about 98% of total alleles, at least about 99% of total alleles, or 100% of total alleles have edited TRAC loci;
(b) less than about 0.1%, less than about 0.2%, less than about 0.3%, less than about 0.4%, less than about 0.5%, less than about 0.6%, less than about 0.7%, less than about 0.8%, less than about 0.9%, or less than about 1% of the T cells are TCR+ T cells;
(c) at least about 5% of total alleles, at least about 10% of total alleles, at least about 15% of total alleles, at least about 20% of total alleles, at least about 25% of total alleles, at least about 30% of total alleles, at least about 35% of total alleles, at least about 40% of total alleles, at least about 45% of total alleles, at least about 50% of total alleles, at least about 55% of total alleles, at least about 60% of total alleles, at least about 65% of total alleles, at least about 70% of total alleles, at least about 75% of total alleles, at least about 80% of total alleles, at least about 85% of total alleles, at least about 90% of total alleles, at least about 95% of total alleles, at least about 96% of total alleles, at least about 97% of total alleles, at least about 98% of total alleles, at least about 99% of total alleles, or 100% of total alleles have edited B2M loci;
(d) at least about 5% of total alleles, at least about 10% of total alleles, at least about 15% of total alleles, at least about 20% of total alleles, at least about 25% of total alleles, at least about 30% of total alleles, at least about 35% of total alleles, at least about 40% of total alleles, at least about 45% of total alleles, at least about 50% of total alleles, at least about 55% of total alleles, at least about 60% of total alleles, at least about 65% of total alleles, at least about 70% of total alleles, at least about 75% of total alleles, at least about 80% of total alleles, at least about 85% of total alleles, at least about 90% of total alleles, at least about 95% of total alleles, at least about 96% of total alleles, at least about 97% of total alleles, at least about 98% of total alleles, at least about 99% of total alleles, or 100% of total alleles have a nucleic acid encoding HLA-E(A) knocked-in at the B2M locus, such as a nucleic acid comprising the sequence set forth in SEQ ID NO: 86, or a nucleic acid encoding HLA-E(A) having an amino acid sequence set forth in SEQ ID NO: 81 or SEQ ID NO: 132;
(e) at least about 5% of total alleles, at least about 10% of total alleles, at least about 15% of total alleles, at least about 20% of total alleles, at least about 25% of total alleles, at least about 30% of total alleles, at least about 35% of total alleles, at least about 40% of total alleles, at least about 45% of total alleles, at least about 50% of total alleles, at least about 55% of total alleles, at least about 60% of total alleles, at least about 65% of total alleles, at least about 70% of total alleles, at least about 75% of total alleles, at least about 80% of total alleles, at least about 85% of total alleles, at least about 90% of total alleles, at least about 95% of total alleles, at least about 96% of total alleles, at least about 97% of total alleles, at least about 98% of total alleles, at least about 99% of total alleles, or 100% of total alleles have a nucleic acid encoding CD19 CAR knocked-in at the TRAC locus, such as a nucleic acid comprising the sequence set forth in SEQ ID NO: 136, or a nucleic acid encoding a CD19 CAR having an amino acid sequence set forth in SEQ ID NO: 78 or SEQ ID NO: 138;
(f) less than about 1 ng, less than about 2 ng, less than about 3 ng, less than about 4 ng, less than about 5 ng, less than about 6 ng, less than about 7 ng, less than about 8 ng, less than about 9 ng, less than about 10 ng, less than about 11 ng, less than about 12 ng, less than about 13 ng, less than about 14 ng, less than about 15 ng, less than about 16 ng, less than about 17 ng, less than about 18 ng, less than about 19 ng, or less than about 20 ng of Cas9 or Cas12a per mL of the composition;
(g) less than about 0.01 fg, less than about 0.02 fg, less than about 0.03 fg, less than about 0.04 fg, less than about 0.05 fg, less than about 0.06 fg, less than about 0.07 fg, less than about 0.08 fg, less than about 0.09 fg, or less than about 1.0 fg of Cas9 or Cas12a per T cell;
(h) less than about 1×10¹⁰, less than about 5×10¹⁰, less than about 1×10¹¹, less than about 5×10¹¹, or less than about 1×10¹² AAV6 capsids per mL of the composition;
(i) ten or fewer, nine or fewer, eight or fewer, seven or fewer, six or fewer, five or fewer, four or fewer, three or fewer, two or fewer, one or fewer, or no verified off-target indel sites at a genomic locus other than TRAC and B2M;
(j) less than about 0.5% of total alleles, less than about 1.0% of total alleles, less than about 1.5% of total alleles, less than about 2.0% of total alleles, less than about 2.5% of total alleles, less than about 3.0% of total alleles, less than about 3.5% of total alleles, or less than about 4.0% of total alleles have AAV6 integration at TRAC locus;
(k) less than about 0.5% of total alleles, less than about 0.6% of total alleles, less than about 0.7% of total alleles, less than about 0.8% of total alleles, less than about 0.9% of total alleles, less than about 1.0% of total alleles, less than about 1.1% of total alleles, less than about 1.2% of total alleles, less than about 1.3% of total alleles, less than about 1.4% of total alleles, less than about 1.5% of total alleles, less than about 1.6% of total alleles, less than about 1.7% of total alleles, less than about 1.8% of total alleles, less than about 1.9% of total alleles, or less than about 2.0% of total alleles have AAV6 integration at B2M locus;
(l) undetectable AAV6 integration at loci other than TRAC and B2M; or
(m) less than about 100 ng, less than about 150 ng, less than about 200 ng, less than about 250 ng, less than about 300 ng, less than about 350 ng, less than about 400 ng, less than about 450 ng, less than about 500 ng, less than about 550 ng, less than about 600 ng, less than about 650 ng, less than about 700 ng, less than about 750 ng, less than about 800 ng, less than about 850 ng, less than about 900 ng, less than about 950 ng, less than about 1000 ng, less than about 1100 ng, less than about 1200 ng, less than about 1300 ng, less than about 1400 ng, or less than about 1500 ng of Strep-Tactin Multimer activation reagent per mL of the composition.

262. The composition of any of embodiments 233-261, wherein the composition comprises a population of T cells wherein the composition has one or more, two or more, three or more, four or more, five or more, six or more, seven or more, eight or more, nine or more, ten or more, eleven or more, twelve or more, thirteen or more, fourteen or more, or all the following attributes:
(i) at least about 70% of the T cells are viable;
(ii) at least about 90% of the T cells are CD2+CD5+;
(iii) at least about 50% of the T cells express CD19 CAR, such as the CD19 CAR having an amino acid sequence set forth in SEQ ID NO: 78 or SEQ ID NO: 138;
(iv) less than about 5% of the T cells are HLA-I+;
(v) the ratio of % TCR+ to % CAR+ T cells is less than about 0.008;
(vi) less than about 5% of total alleles have translocation between TRAC and B2M; or
(vii) at least about 20,000,000 viable CD2+CD5+CD19 CAR+ cells per mL of the composition;
(viii) the T cells are from a single healthy donor aged 18 to 35 years old;
(ix) the T cells are from a single healthy donor having a body mass index (BMI) less than 30 kg/m²;
(x) no detected bacterial growth;
(xi) no more than about 13.3 EU/mL endotoxin;
(xii) no detected mycoplasma;
(xiii) no cytokine-independent growth;
(xiv) no significant unexpected karyotype;
(xv) negative for the presence of HIV, HTLV, HAV, HBV, HCV, CMV, EBV, HHV6, HHV7, HHV8, and B19.

263. The composition of any of embodiments 233-262, wherein the composition comprises a population of T cells, wherein the T cells are from a single healthy donor aged 18 to 35 years old and having a body mass index (BMI) less than 30 kg/m², and wherein the composition has one or more, two or more, three or more, four or more, five or more, six or more, seven or more, eight or more, nine or more, ten or more, eleven or more, twelve or more, or all the following attributes:
(i) at least about 70% of the T cells are viable;
(ii) at least about 90% of the T cells are CD2+CD5+;
(iii) at least about 50% of the T cells express CD19 CAR, such as the CD19 CAR having an amino acid sequence set forth in SEQ ID NO: 78 or SEQ ID NO: 138.
(iv) less than about 5% of the T cells are HLA-I+;
(v) the ratio of % TCR+ to % CAR+ T cells is less than about 0.008;
(vi) less than about 5% of total alleles have translocation between TRAC and B2M;
(vii) at least about 20,000,000 viable CD2+CD5+CD19 CAR+ cells per mL of the composition;
(viii) no detected bacterial growth;
(ix) no more than about 13.3 EU/mL endotoxin;
(x) no detected mycoplasma;
(xi) no cytokine-independent growth;
(xii) no significant unexpected karyotype; or
(xiii) negative for the presence of HIV, HTLV, HAV, HBV, HCV, CMV, EBV, HHV6, HHV7, HHV8, and B19.

264. The composition of any of embodiments 233-263, wherein the composition comprises a population of T cells, wherein the T cells are from a single healthy donor aged 18 to 35 years old and having a body mass index (BMI) less than 30 kg/m², and wherein the composition has one or more, two or more, three or more, four or more, five or more, six or more, seven or more, eight or more, nine or more, ten or more, eleven or more, twelve or more, or all the following attributes:
(i) at least about 70% of the T cells are viable;
(ii) at least about 90% of the T cells are CD2+CD5+;
(iii) at least about 50% of the T cells express CD19 CAR, such as the CD19 CAR having an amino acid sequence set forth in SEQ ID NO: 78 or SEQ ID NO: 138;
(iv) less than about 5% of the T cells are HLA-I+;
(v) the ratio of % TCR+ to % CAR+ T cells is less than about 0.008;
(vi) less than about 5% of total alleles have translocation between TRAC and B2M;
(vii) at least about 20,000,000 viable CD2+CD5+CD19 CAR+ cells per mL of the composition;
(viii) no detected bacterial growth;
(ix) no more than about 13.3 EU/mL endotoxin;
(x) no detected mycoplasma;
(xi) no cytokine-independent growth;
(xii) no significant unexpected karyotype; or
(xiii) negative for the presence of HIV, HTLV, HAV, HBV, HCV, CMV, EBV, HHV6, HHV7, HHV8, and B19, and wherein the composition has one or more of the following attributes:
(a) at least about 70% of total alleles, at least about 75% of total alleles, at least about 80% of total alleles, at least about 85% of total alleles, at least about 90% of total alleles, at least about 95% of total alleles, at least about 96% of total alleles, at least about 97% of total alleles, at least about 98% of total alleles, at least about 99% of total alleles, or 100% of total alleles have edited TRAC loci;
(b) less than about 0.1%, less than about 0.2%, less than about 0.3%, less than about 0.4%, less than about 0.5%, less than about 0.6%, less than about 0.7%, less than about 0.8%, less than about 0.9%, or less than about 1% of the T cells are TCR+ T cells;
(c) at least about 70% of total alleles, at least about 75% of total alleles, at least about 80% of total alleles, at least about 85% of total alleles, at least about 90% of total alleles, at least about 95% of total alleles, at least about 96% of total alleles, at least about 97% of total alleles, at least about 98% of total alleles, at least about 99% of total alleles, or 100% of total alleles have edited B2M loci;
(d) at least about 25% of total alleles, at least about 30% of total alleles, at least about 35% of total alleles, at least about 40% of total alleles, at least about 45% of total alleles, at least about 50% of total alleles, at least about 55% of total alleles, at least about 60% of total alleles, at least about 65% of total alleles, at least about 70% of total alleles, at least about 75% of total alleles, at least about 80% of total alleles, at least about 85% of total alleles, at least about 90% of total alleles, at least about 95% of total alleles, at least about 96% of total alleles, at least about 97% of total alleles, at least about 98% of total alleles, at least about 99% of total alleles, or 100% of total alleles have a nucleic acid encoding HLA-E(A) knocked-in at the B2M locus, such as a nucleic acid comprising the sequence set forth in SEQ ID NO: 86, or a nucleic acid encoding HLA-E(A) having an amino acid sequence set forth in SEQ ID NO: 81 or SEQ ID NO: 132;
(e) at least about 25% of total alleles, at least about 30% of total alleles, at least about 35% of total alleles, at least about 40% of total alleles, at least about 45% of total alleles, at least about 50% of total alleles, at least about 55% of total alleles, at least about 60% of total alleles, at least about 65% of total alleles, at least about 70% of total alleles, at least about 75% of total alleles, at least about 80% of total alleles, at least about 85% of total alleles, at least about 90% of total alleles, at least about 95% of total alleles, at least about 96% of total alleles, at least about 97% of total alleles, at least about 98% of total alleles, at least about 99% of total alleles, or 100% of total alleles have a nucleic acid encoding CD19 CAR knocked-in at the TRAC locus, such as a nucleic acid comprising the sequence set forth in SEQ ID NO: 136, or a nucleic acid encoding a CD19 CAR having an amino acid sequence set forth in SEQ ID NO: 78 or SEQ ID NO: 138;
(f) less than about 1 ng, less than about 2 ng, less than about 3 ng, less than about 4 ng, less than about 5 ng, less than about 6 ng, less than about 7 ng, less than about 8 ng, less than about 9 ng, less than about 10 ng, less than about 11 ng, less than about 12 ng, less than about 13 ng, less than about 14 ng, less than about 15 ng, less than about 16 ng, less than about 17 ng, less than about 18 ng, less than about 19 ng, or less than about 20 ng of Cas9 or Cas12a per mL of the composition;
(g) less than about 0.01 fg, less than about 0.02 fg, less than about 0.03 fg, less than about 0.04 fg, less than about 0.05 fg, less than about 0.06 fg, less than about 0.07 fg, less than about 0.08 fg, less than about 0.09 fg, or less than about 1.0 fg of Cas9 or Cas12a per T cell;
(h) less than about 1×10¹⁰, less than about 5×10¹⁰, less than about 1×10¹¹, less than about 5×10¹¹, or less than about 1×10¹² AAV6 capsids per mL of the composition;
(i) ten or fewer, nine or fewer, eight or fewer, seven or fewer, six or fewer, five or fewer, four or fewer, three or fewer, two or fewer, one or fewer, or no verified off-target indel sites at a genomic locus other than TRAC and B2M;
(j) less than about 0.5% of total alleles, less than about 1.0% of total alleles, less than about 1.5% of total alleles, less than about 2.0% of total alleles, less than about 2.5% of total alleles, less than about 3.0% of total alleles, less than about 3.5% of total alleles, or less than about 4.0% of total alleles have AAV6 integration at TRAC locus;
(k) less than about 0.5% of total alleles, less than about 0.6% of total alleles, less than about 0.7% of total alleles, less than about 0.8% of total alleles, less than about 0.9% of total alleles, less than about 1.0% of total alleles, less than about 1.1% of total alleles, less than about 1.2% of total alleles, less than about 1.3% of total alleles, less than about 1.4% of total alleles, less than about 1.5% of total alleles, less than about 1.6% of total alleles, less than about 1.7% of total alleles, less than about 1.8% of total alleles, less than about 1.9% of total alleles, or less than about 2.0% of total alleles have AAV6 integration at B2M locus;
(l) undetectable AAV6 integration at loci other than TRAC and B2M; or
(m) less than about 100 ng, less than about 150 ng, less than about 200 ng, less than about 250 ng, less than about 300 ng, less than about 350 ng, less than about 400 ng, less than about 450 ng, less than about 500 ng, less than about 550 ng, less than about 600 ng, less than about 650 ng, less than about 700 ng, less than about 750 ng, less than about 800 ng, less than about 850 ng, less than about 900 ng, less than about 950 ng, less than about 1000 ng, less than about 1100 ng, less than about 1200 ng, less than about 1300 ng, less than about 1400 ng, or less than about 1500 ng of Strep-Tactin Multimer activation reagent per mL of the composition.

265. The composition of any of embodiments 233-264, wherein the composition comprises a population of T cells, wherein the T cells are from a single healthy donor aged 18 to 35 years old and having a body mass index (BMI) less than 30 kg/m², and wherein the composition has one or more, two or more, three or more, four or more, five or more, six or more, seven or more, eight or more, nine or more, ten or more, eleven or more, twelve or more, or all the following attributes:
(i) at least about 70% of the T cells are viable;
(ii) at least about 90% of the T cells are CD2+CD5+;
(iii) at least about 50% of the T cells express CD19 CAR, such as the CD19 CAR having an amino acid sequence set forth in SEQ ID NO: 78 or SEQ ID NO: 138;
(iv) less than about 5% of the T cells are HLA-I+;
(v) the ratio of % TCR+ to % CAR+ T cells is less than about 0.008;
(vi) less than about 5% of total alleles have translocation between TRAC and B2M;
(vii) at least about 20,000,000 viable CD2+CD5+CD19 CAR+ cells per mL of the composition;
(viii) no detected bacterial growth;
(ix) no more than about 13.3 EU/mL endotoxin;
(x) no detected mycoplasma;
(xi) no cytokine-independent growth;
(xii) no significant unexpected karyotype; or
(xiii) negative for the presence of HIV, HTLV, HAV, HBV, HCV, CMV, EBV, HHV6, HHV7, HHV8, and B19, and wherein the composition has one or more of the following attributes:
(a) at least about 90% of total alleles have edited TRAC loci;
(b) less than about 0.5% of the T cells are TCR+ T cells;
(c) at least about 90% of total alleles have edited B2M loci;
(d) at least about 50% of total alleles have a nucleic acid encoding HLA-E(A) knocked-in at the B2M locus, such as a nucleic acid comprising the sequence set forth in SEQ ID NO: 86, or a nucleic acid encoding HLA-E(A) having an amino acid sequence set forth in SEQ ID NO: 81 or SEQ ID NO: 132;
(e) at least about 50% of total alleles have a nucleic acid encoding CD19 CAR knocked-in at the TRAC locus, such as a nucleic acid comprising the sequence set forth in SEQ ID NO: 136, or a nucleic acid encoding a CD19 CAR having an amino acid sequence set forth in SEQ ID NO: 78 or SEQ ID NO: 138;
(f) less than about 2 ng of Cas9 or Cas12a per mL of the composition;
(g) less than about 0.05 fg of Cas9 or Cas12a per T cell;
(h) less than about 5×10¹¹ AAV6 capsids per mL of the composition;
(i) no verified off-target indel sites at a genomic locus other than TRAC and B2M;
(j) less than about 2.0% of total alleles have AAV6 integration at TRAC locus;
(k) less than about 1.0% of total alleles have AAV6 integration at B2M locus;
(l) undetectable AAV6 integration at loci other than TRAC and B2M; or
(m) less than about 900 ng of Strep-Tactin Multimer activation reagent per mL of the composition.

266. The composition of any of embodiments 233-265, wherein the composition comprises a population of T cells, wherein the T cells are from a single healthy donor aged 18 to 35 years old and having a body mass index (BMI) less than 30 kg/m², and wherein the composition has one or more, two or more, three or more, four or more, five or more, six or more, seven or more, eight or more, nine or more, ten or more, eleven or more, twelve or more, or all the following attributes:
(i) at least about 80%, at least about 85%, or at least about 90% of the T cells are viable;
(ii) at least about 95%, at least about 96%, at least about 97%, at least about 98%, or at least about 99% of the T cells are CD2+CD5+;
(iii) at least about 50% of the T cells express CD19 CAR, such as the CD19 CAR having an amino acid sequence set forth in SEQ ID NO: 78 or SEQ ID NO: 138;
(iv) less than about 5% of the T cells are HLA-I+;
(v) the ratio of % TCR+ to % CAR+ T cells is less than about 0.008;
(vi) less than about 5% of total alleles have translocation between TRAC and B2M;
(vii) at least about 20,000,000 viable CD2+CD5+CD19 CAR+ cells per mL of the composition;
(viii) no detected bacterial growth;
(ix) no more than about 13.3 EU/mL endotoxin;
(x) no detected mycoplasma;
(xi) no cytokine-independent growth;
(xii) no significant unexpected karyotype; or
(xiii) negative for the presence of HIV, HTLV, HAV, HBV, HCV, CMV, EBV, HHV6, HHV7, HHV8, and B19.

267. The composition of any of embodiments 233-266, wherein the composition comprises a population of T cells, wherein the T cells are from a single healthy donor aged 18 to 35 years old and having a body mass index (BMI) less than 30 kg/m², and wherein the composition has one or more, two or more, three or more, four or more, five or more, six or more, seven or more, eight or more, nine or more, ten or more, eleven or more, twelve or more, or all the following attributes:
(i) at least about 80%, at least about 85%, or at least about 90% of the T cells are viable;
(ii) at least about 95%, at least about 96%, at least about 97%, at least about 98%, or at least about 99% of the T cells are CD2+CD5+;
(iii) at least about 50% of the T cells express CD19 CAR, such as the CD19 CAR having an amino acid sequence set forth in SEQ ID NO: 78 or SEQ ID NO: 138;
(iv) less than about 5% of the T cells are HLA-I+;
(v) the ratio of % TCR+ to % CAR+ T cells is less than about 0.008;
(vi) less than about 5% of total alleles have translocation between TRAC and B2M;
(vii) at least about 20,000,000 viable CD2+CD5+CD19 CAR+ cells per mL of the composition;
(viii) no detected bacterial growth;
(ix) no more than about 13.3 EU/mL endotoxin;
(x) no detected mycoplasma;
(xi) no cytokine-independent growth;
(xii) no significant unexpected karyotype; or
(xiii) negative for the presence of HIV, HTLV, HAV, HBV, HCV, CMV, EBV, HHV6, HHV7, HHV8, and B19, and wherein the composition has one or more of the following attributes:
(a) at least about 70% of total alleles, at least about 75% of total alleles, at least about 80% of total alleles, at least about 85% of total alleles, at least about 90% of total alleles, at least about 95% of total alleles, at least about 96% of total alleles, at least about 97% of total alleles, at least about 98% of total alleles, at least about 99% of total alleles, or 100% of total alleles have edited TRAC loci;
(b) less than about 0.1%, less than about 0.2%, less than about 0.3%, less than about 0.4%, less than about 0.5%, less than about 0.6%, less than about 0.7%, less than about 0.8%, less than about 0.9%, or less than about 1% of the T cells are TCR+ T cells;
(c) at least about 70% of total alleles, at least about 75% of total alleles, at least about 80% of total alleles, at least about 85% of total alleles, at least about 90% of total alleles, at least about 95% of total alleles, at least about 96% of total alleles, at least about 97% of total alleles, at least about 98% of total alleles, at least about 99% of total alleles, or 100% of total alleles have edited B2M loci;
(d) at least about 25% of total alleles, at least about 30% of total alleles, at least about 35% of total alleles, at least about 40% of total alleles, at least about 45% of total alleles, at least about 50% of total alleles, at least about 55% of total alleles, at least about 60% of total alleles, at least about 65% of total alleles, at least about 70% of total alleles, at least about 75% of total alleles, at least about 80% of total alleles, at least about 85% of total alleles, at least about 90% of total alleles, at least about 95% of total alleles, at least about 96% of total alleles, at least about 97% of total alleles, at least about 98% of total alleles, at least about 99% of total alleles, or 100% of total alleles have a nucleic acid encoding HLA-E(A) knocked-in at the B2M locus, such as a nucleic acid comprising the sequence set forth in SEQ ID NO: 86, or a nucleic acid encoding HLA-E(A) having an amino acid sequence set forth in SEQ ID NO: 81 or SEQ ID NO: 132;
(e) at least about 25% of total alleles, at least about 30% of total alleles, at least about 35% of total alleles, at least about 40% of total alleles, at least about 45% of total alleles, at least about 50% of total alleles, at least about 55% of total alleles, at least about 60% of total alleles, at least about 65% of total alleles, at least about 70% of total alleles, at least about 75% of total alleles, at least about 80% of total alleles, at least about 85% of total alleles, at least about 90% of total alleles, at least about 95% of total alleles, at least about 96% of total alleles, at least about 97% of total alleles, at least about 98% of total alleles, at least about 99% of total alleles, or 100% of total alleles have a nucleic acid encoding CD19 CAR knocked-in at the TRAC locus, such as a nucleic acid comprising the sequence set forth in SEQ ID NO: 136, or a nucleic acid encoding a CD19 CAR having an amino acid sequence set forth in SEQ ID NO: 78 or SEQ ID NO: 138;
(f) less than about 1 ng, less than about 2 ng, less than about 3 ng, less than about 4 ng, less than about 5 ng, less than about 6 ng, less than about 7 ng, less than about 8 ng, less than about 9 ng, less than about 10 ng, less than about 11 ng, less than about 12 ng, less than about 13 ng, less than about 14 ng, less than about 15 ng, less than about 16 ng, less than about 17 ng, less than about 18 ng, less than about 19 ng, or less than about 20 ng of Cas9 or Cas12a per mL of the composition;
(g) less than about 0.01 fg, less than about 0.02 fg, less than about 0.03 fg, less than about 0.04 fg, less than about 0.05 fg, less than about 0.06 fg, less than about 0.07 fg, less than about 0.08 fg, less than about 0.09 fg, or less than about 1.0 fg of Cas9 or Cas12a per T cell;
(h) less than about 1×10¹⁰, less than about 5×10¹⁰, less than about 1×10¹¹, less than about 5×10¹¹, or less than about 1×10¹² AAV6 capsids per mL of the composition;
(i) ten or fewer, nine or fewer, eight or fewer, seven or fewer, six or fewer, five or fewer, four or fewer, three or fewer, two or fewer, one or fewer, or no verified off-target indel sites at a genomic locus other than TRAC and B2M;
(j) less than about 0.5% of total alleles, less than about 1.0% of total alleles, less than about 1.5% of total alleles, less than about 2.0% of total alleles, less than about 2.5% of total alleles, less than about 3.0% of total alleles, less than about 3.5% of total alleles, or less than about 4.0% of total alleles have AAV6 integration at TRAC locus;
(k) less than about 0.5% of total alleles, less than about 0.6% of total alleles, less than about 0.7% of total alleles, less than about 0.8% of total alleles, less than about 0.9% of total alleles, less than about 1.0% of total alleles, less than about 1.1% of total alleles, less than about 1.2% of total alleles, less than about 1.3% of total alleles, less than about 1.4% of total alleles, less than about 1.5% of total alleles, less than about 1.6% of total alleles, less than about 1.7% of total alleles, less than about 1.8% of total alleles, less than about 1.9% of total alleles, or less than about 2.0% of total alleles have AAV6 integration at B2M locus;
(l) undetectable AAV6 integration at loci other than TRAC and B2M; or
(m) less than about 100 ng, less than about 150 ng, less than about 200 ng, less than about 250 ng, less than about 300 ng, less than about 350 ng, less than about 400 ng, less than about 450 ng, less than about 500 ng, less than about 550 ng, less than about 600 ng, less than about 650 ng, less than about 700 ng, less than about 750 ng, less than about 800 ng, less than about 850 ng, less than about 900 ng, less than about 950 ng, less than about 1000 ng, less than about 1100 ng, less than about 1200 ng, less than about 1300 ng, less than about 1400 ng, or less than about 1500 ng of Strep-Tactin Multimer activation reagent per mL of the composition.

268. The composition of any of embodiments 233-267, wherein the composition comprises a population of T cells, wherein the T cells are from a single healthy donor aged 18 to 35 years old and having a body mass index (BMI) less than 30 kg/m², and wherein the composition has one or more, two or more, three or more, four or more, five or more, six or more, seven or more, eight or more, nine or more, ten or more, eleven or more, twelve or more, or all the following attributes:
(i) at least about 80%, at least about 85%, or at least about 90% of the T cells are viable;
(ii) at least about 95%, at least about 96%, at least about 97%, at least about 98%, or at least about 99% of the T cells are CD2+CD5+;
(iii) at least about 50% of the T cells express CD19 CAR, such as the CD19 CAR having an amino acid sequence set forth in SEQ ID NO: 78 or SEQ ID NO: 138;
(iv) less than about 5% of the T cells are HLA-I+;
(v) the ratio of % TCR+ to % CAR+ T cells is less than about 0.008;
(vi) less than about 5% of total alleles have translocation between TRAC and B2M;
(vii) at least about 20,000,000 viable CD2+CD5+CD19 CAR+ cells per mL of the composition;
(viii) no detected bacterial growth;
(ix) no more than about 13.3 EU/mL endotoxin;
(x) no detected mycoplasma;
(xi) no cytokine-independent growth;
(xii) no significant unexpected karyotype; or
(xiii) negative for the presence of HIV, HTLV, HAV, HBV, HCV, CMV, EBV, HHV6, HHV7, HHV8, and B19, and wherein the composition has one or more of the following attributes:
(a) at least about 90% of total alleles have edited TRAC loci;
(b) less than about 0.5% of the T cells are TCR+ T cells;
(c) at least about 90% of total alleles have edited B2M loci;
(d) at least about 50% of total alleles have a nucleic acid encoding HLA-E(A) knocked-in at the B2M locus, such as a nucleic acid comprising the sequence set forth in SEQ ID NO: 86, or a nucleic acid encoding HLA-E(A) having an amino acid sequence set forth in SEQ ID NO: 81 or SEQ ID NO: 132;
(e) at least about 50% of total alleles have a nucleic acid encoding CD19 CAR knocked-in at the TRAC locus, such as a nucleic acid comprising the sequence set forth in SEQ ID NO: 136, or a nucleic acid encoding a CD19 CAR having an amino acid sequence set forth in SEQ ID NO: 78 or SEQ ID NO: 138;
(f) less than about 2 ng of Cas9 or Cas12a per mL of the composition;
(g) less than about 0.05 fg of Cas9 or Cas12a per T cell;
(h) less than about 5×10¹¹ AAV6 capsids per mL of the composition;
(i) no verified off-target indel sites at a genomic locus other than TRAC and B2M;
(j) less than about 2.0% of total alleles have AAV6 integration at TRAC locus;
(k) less than about 1.0% of total alleles have AAV6 integration at B2M locus;
(l) undetectable AAV6 integration at loci other than TRAC and B2M; or
(m) less than about 900 ng of Strep-Tactin Multimer activation reagent per mL of the composition.

269. The composition of any of embodiments 233-268, wherein the T cells comprise (i) a modified TRAC locus comprising a transgene sequence comprising the sequence set forth in SEQ ID NO: 136, and (ii) a modified B2M locus comprising a transgene sequence comprising the sequence set forth in SEQ ID NO: 86.

270. The composition of any of embodiments 233-268, wherein the T cells comprise (i) a modified TRAC locus comprising a transgene sequence comprising the sequence set forth in SEQ ID NO: 94, and (ii) a modified B2M locus comprising a transgene sequence comprising the sequence set forth in SEQ ID NO: 137.

271. A composition comprising a population of T cells, wherein the composition has the following attributes:
(i) at least about 50%, at least about 55%, at least about 60%, at least about 65%, at least about 70%, at least about 75%, at least about 80%, at least about 85%, at least about 90%, or at least about 95% of the T cells are viable;
(ii) at least about 70%, at least about 75%, at least about 80%, at least about 85%, or at least about 90% of the T cells are CD2+CD5+;
(iii) at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the T cells express CD19 CAR, such as the CD19 CAR having an amino acid sequence set forth in SEQ ID NO: 78 or SEQ ID NO: 138;
(iv) less than about 2%, less than about 3%, less than about 4%, less than about 5%, less than about 6%, less than about 7%, less than about 8%, less than about 9%, or less than about 10% of the T cells are HLA-I+;
(v) the ratio of % TCR+ to % CAR+ T cells is less than about 0.005, less than about 0.006, less than about 0.007, less than about 0.008, less than about 0.009, or less than about 0.01;
(vi) less than about 1% of total alleles, less than about 2% of total alleles, less than about 3% of total alleles, less than about 4% of total alleles, less than about 5% of total alleles, less than about 6% of total alleles, less than about 7% of total alleles, less than about 8% of total alleles, less than about 9% of total alleles, or less than about 10% of total alleles have translocation between TRAC and B2M; and
(vii) at least about 5,000,000, at least about 10,000,000, at least about 15,000,000, at least about 20,000,000, at least about 25,000,000, at least about 30,000,000, at least about 35,000,000, at least about 40,000,000, at least about 45,000,000, or at least about 50,000,000 viable CD2+CD5+CD19 CAR+ cells per mL of the composition;
wherein the T cells (i) are from a single healthy donor aged 18 to 35 years old and having a body mass index (BMI) less than 30 kg/m², and (ii) comprise (a) a modified TRAC locus comprising a transgene sequence comprising the sequence set forth in SEQ ID NO: 136, and (b) a modified B2M locus comprising a transgene sequence comprising the sequence set forth in SEQ ID NO: 86.

272. A composition comprising a population of T cells, wherein the composition has the following attributes:
(i) at least about 50%, at least about 55%, at least about 60%, at least about 65%, at least about 70%, at least about 75%, at least about 80%, at least about 85%, at least about 90%, or at least about 95% of the T cells are viable;
(ii) at least about 70%, at least about 75%, at least about 80%, at least about 85%, or at least about 90% of the T cells are CD2+CD5+;
(iii) at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% of the T cells express CD19 CAR, such as the CD19 CAR having an amino acid sequence set forth in SEQ ID NO: 78 or SEQ ID NO: 138;
(iv) less than about 2%, less than about 3%, less than about 4%, less than about 5%, less than about 6%, less than about 7%, less than about 8%, less than about 9%, or less than about 10% of the T cells are HLA-I+;
(v) the ratio of % TCR+ to % CAR+ T cells is less than about 0.005, less than about 0.006, less than about 0.007, less than about 0.008, less than about 0.009, or less than about 0.01;
(vi) less than about 1% of total alleles, less than about 2% of total alleles, less than about 3% of total alleles, less than about 4% of total alleles, less than about 5% of total alleles, less than about 6% of total alleles, less than about 7% of total alleles, less than about 8% of total alleles, less than about 9% of total alleles, or less than about 10% of total alleles have translocation between TRAC and B2M; and
(vii) at least about 5,000,000, at least about 10,000,000, at least about 15,000,000, at least about 20,000,000, at least about 25,000,000, at least about 30,000,000, at least about 35,000,000, at least about 40,000,000, at least about 45,000,000, or at least about 50,000,000 viable CD2+CD5+CD19 CAR+ cells per mL of the composition; wherein the T cells (i) are from a single healthy donor aged 18 to 35 years old and having a body mass index (BMI) less than 30 kg/m², and (ii) comprise (a) a modified TRAC locus comprising a transgene sequence comprising the sequence set forth in SEQ ID NO: 94, and (b) a modified B2M locus comprising a transgene sequence comprising the sequence set forth in SEQ ID NO: 137.

273. A composition comprising a population of T cells, wherein the composition has the following attributes:
(i) at least about 70% of the T cells are viable;
(ii) at least about 90% of the T cells are CD2+CD5+;
(iii) at least about 50% of the T cells express a CD19 CAR having an amino acid sequence set forth in SEQ ID NO: 78 or SEQ ID NO: 138;
(iv) less than about 5% of the T cells are HLA-I+;
(v) the ratio of % TCR+ to % CAR+ T cells is less than about 0.008;
(vi) less than about 5% of total alleles have translocation between TRAC and B2M; and
(vii) at least about 20,000,000 viable CD2+CD5+CD19 CAR+ cells per mL of the composition; wherein the T cells (i) are from a single healthy donor aged 18 to 35 years old and having a body mass index (BMI) less than 30 kg/m², and (ii) comprise (a) a modified TRAC locus comprising a transgene sequence comprising the sequence set forth in SEQ ID NO: 136, and (b) a modified B2M locus comprising a transgene sequence comprising the sequence set forth in SEQ ID NO: 86.

274. A composition comprising a population of T cells, wherein the composition has the following attributes:
(i) at least about 70% of the T cells are viable;
(ii) at least about 90% of the T cells are CD2+CD5+;
(iii) at least about 50% of the T cells express a CD19 CAR having an amino acid sequence set forth in SEQ ID NO: 78 or SEQ ID NO: 138;
(iv) less than about 5% of the T cells are HLA-I+;
(v) the ratio of % TCR+ to % CAR+ T cells is less than about 0.008;
(vi) less than about 5% translocation between TRAC and B2M; and
(vii) at least about 20,000,000 viable CD2+CD5+CD19 CAR+ cells per mL of the composition; wherein the T cells (i) are from a single healthy donor between aged 18 to 35 years old and having a body mass index (BMI) less than 30 kg/m², and (ii) comprise (a) a modified TRAC locus comprising a transgene sequence comprising the sequence set forth in SEQ ID NO: 94, and (b) a modified B2M locus comprising a transgene sequence comprising the sequence set forth in SEQ ID NO: 137.

275. A composition comprising a population of T cells, wherein the composition has the following attributes:
(i) at least about 80%, at least about 85%, or at least about 90% of the T cells are viable;
(ii) at least about 95%, at least about 96%, at least about 97%, at least about 98%, or at least about 99% of the T cells are CD2+CD5+;
(iii) at least about 50% of the T cells express CD19 CAR, such as the CD19 CAR having an amino acid sequence set forth in SEQ ID NO: 78 or SEQ ID NO: 138;
(iv) less than about 5% of the T cells are HLA-I+;
(v) the ratio of % TCR+ to % CAR+ T cells is less than about 0.008;
(vi) less than about 5% of total alleles have translocation between TRAC and B2M; and
(vii) at least about 20,000,000 viable CD2+CD5+CD19 CAR+ cells per mL of the composition; wherein the T cells (i) are from a single healthy donor aged 18 to 35 years old and having a body mass index (BMI) less than 30 kg/m², and (ii) comprise (a) a modified TRAC locus comprising a transgene sequence comprising the sequence set forth in SEQ ID NO: 94, and (b) a modified B2M locus comprising a transgene sequence comprising the sequence set forth in SEQ ID NO: 137.

276. A composition comprising a population of T cells, wherein the composition has the following attributes:
(i) at least about 80%, at least about 85%, or at least about 90% of the T cells are viable;
(ii) at least about 95%, at least about 96%, at least about 97%, at least about 98%, or at least about 99% of the T cells are CD2+CD5+;
(iii) at least about 50% of the T cells express CD19 CAR, such as the CD19 CAR having an amino acid sequence set forth in SEQ ID NO: 78 or SEQ ID NO: 138;
(iv) less than about 5% of the T cells are HLA-I+;
(v) the ratio of % TCR+ to % CAR+ T cells is less than about 0.008;
(vi) less than about 5% of total alleles have translocation between TRAC and B2M; and
(vii) at least about 20,000,000 viable CD2+CD5+CD19 CAR+ cells per mL of the composition; wherein the T cells (i) are from a single healthy donor aged 18 to 35 years old and having a body mass index (BMI) less than 30 kg/m², and (ii) comprise (a) a modified TRAC locus comprising a transgene sequence comprising the sequence set forth in SEQ ID NO: 94, and (b) a modified B2M locus comprising a transgene sequence comprising the sequence set forth in SEQ ID NO: 137; and wherein the composition has one or more of the following attributes:
(a) at least about 90% of total alleles have edited TRAC loci;
(b) less than about 0.5% of the T cells are TCR+ T cells;
(c) at least about 90% of total alleles have edited B2M loci;
(d) at least about 50% of total alleles have a nucleic acid encoding HLA-E(A) knocked-in at the B2M locus, such as a nucleic acid comprising the sequence set forth in SEQ ID NO: 86, or a nucleic acid encoding HLA-E(A) having an amino acid sequence set forth in SEQ ID NO: 81 or SEQ ID NO: 132;
(e) at least about 50% of total alleles have a nucleic acid encoding CD19 CAR knocked-in at the TRAC locus, such as a nucleic acid comprising the sequence set forth in SEQ ID NO: 136, or a nucleic acid encoding a CD19 CAR having an amino acid sequence set forth in SEQ ID NO: 78 or SEQ ID NO: 138;
(f) less than about 2 ng of Cas9 or Cas12a per mL of the composition;
(g) less than about 0.05 fg of Cas9 or Cas12a per T cell;
(h) less than about 5×10¹¹ AAV6 capsids per mL of the composition;
(i) no verified off-target indel sites at a genomic locus other than TRAC and B2M;
(j) less than about 2.0% of total alleles have AAV6 integration at TRAC locus;
(k) less than about 1.0% of total alleles have AAV6 integration at B2M locus;
(l) undetectable AAV6 integration at loci other than TRAC and B2M; or
(m) less than about 900 ng of Strep-Tactin Multimer activation reagent per mL of the composition.

277. A composition comprising a population of T cells, wherein the composition has one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, twenty-four or more, twenty-five or more, or all the following attributes:
(i) at least about 80%, at least about 85%, or at least about 90% of the T cells are viable;
(ii) at least about 95%, at least about 96%, at least about 97%, at least about 98%, or at least about 99% of the T cells are CD2+CD5+;
(iii) at least about 50% of the T cells express CD19 CAR, such as the CD19 CAR having an amino acid sequence set forth in SEQ ID NO: 78 or SEQ ID NO: 138;
(iv) less than about 5% of the T cells are HLA-I+;
(v) the ratio of % TCR+ to % CAR+ T cells is less than about 0.008;
(vi) less than about 5% of total alleles have translocation between TRAC and B2M;
(vii) at least about 20,000,000 viable CD2+CD5+CD19 CAR+ cells per mL of the composition;
(viii) no detected bacterial growth;
(ix) no more than about 13.3 EU/mL endotoxin;
(x) no detected mycoplasma;
(xi) no cytokine-independent growth;
(xii) no significant unexpected karyotype;
(xiii) negative for the presence of HIV, HTLV, HAV, HBV, HCV, CMV, EBV, HHV6, HHV7, HHV8, and B19;
(xiv) at least about 90% of total alleles have edited TRAC loci;
(xv) less than about 0.5% of the T cells are TCR+ T cells;
(xvi) at least about 90% of total alleles have edited B2M loci;
(xvii) at least about 50% of total alleles have a nucleic acid encoding HLA-E(A) knocked-in at the B2M locus, such as a nucleic acid comprising the sequence set forth in SEQ ID NO: 86, or a nucleic acid encoding HLA-E(A) having an amino acid sequence set forth in SEQ ID NO: 81 or SEQ ID NO: 132;
(xviii) at least about 50% of total alleles have a nucleic acid encoding CD19 CAR knocked-in at the TRAC locus, such as a nucleic acid comprising the sequence set forth in SEQ ID NO: 136, or a nucleic acid encoding a CD19 CAR having an amino acid sequence set forth in SEQ ID NO: 78 or SEQ ID NO: 138;
(xix) less than about 2 ng of Cas9 or Cas12a per mL of the composition;
(xx) less than about 0.05 fg of Cas9 or Cas12a per T cell;
(xxi) less than about 5×10¹¹ AAV6 capsids per mL of the composition;
(xxii) no verified off-target indel sites at a genomic locus other than TRAC and B2M;
(xxiii) less than about 2.0% of total alleles have AAV6 integration at TRAC locus;
(xxiv) less than about 1.0% of total alleles have AAV6 integration at B2M locus;
(xxv) undetectable AAV6 integration at loci other than TRAC and B2M; or
(xxvi) less than about 900 ng of Strep-Tactin Multimer activation reagent per mL of the composition; wherein the T cells (i) are from a single healthy donor aged 18 to 35 years old and having a body mass index (BMI) less than 30 kg/m², and (ii) comprise (a) a modified TRAC locus comprising a transgene sequence comprising the sequence set forth in SEQ ID NO: 94, and (b) a modified B2M locus comprising a transgene sequence comprising the sequence set forth in SEQ ID NO: 137.

278. A composition comprising a population of T cells, wherein the composition has one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, or all the following attributes:
(i) at least about 70% of the T cells are viable;
(ii) at least about 10% of total alleles have edited TRAC loci;
(iii) at least about 10% of total alleles have edited B2M loci;
(iv) at least about 10% of total alleles have a nucleic acid encoding HLA-E(A) knocked-in at the B2M locus, such as a nucleic acid comprising the sequence set forth in SEQ ID NO: 86, or a nucleic acid encoding HLA-E(A) having an amino acid sequence set forth in SEQ ID NO: 81 or SEQ ID NO: 132;
(v) at least about 10% of total alleles have a nucleic acid encoding CD19 CAR knocked-in at the TRAC locus, such as a nucleic acid comprising the sequence set forth in SEQ ID NO: 136, or a nucleic acid encoding a CD19 CAR having an amino acid sequence set forth in SEQ ID NO: 78 or SEQ ID NO: 138;
(vi) at least about 90% of the T cells are CD2+CD5+;
(vii) at least about 50% of the T cells are CD2+CD5+ and express CD19 CAR, such as the CD19 CAR having an amino acid sequence set forth in SEQ ID NO: 78 or SEQ ID NO: 138;
(viii) at least about 7,500,000 viable CD2+CD5+CD19 CAR+ cells per mL of the composition;
(ix) no detected bacterial growth;
(x) less than about 5 EU/mL endotoxin;
(xi) no detected mycoplasma;
(xii) less than about 70,000 TCR+ cells/kg;
(xiii) no cytokine-independent growth;
(xiv) no significant unexpected karyotype
(xv) less than about 5% of total alleles have translocation between TRAC and B2M;
(xvi) negative for the presence of HIV-1, HIV-2, HTLV-1, HTLV-2, HAV, HBV, HCV, CMV, EBV, HHV6, HHV7, HHV8, and B19;
(xvii) the T cells are from a healthy donor aged 18 to 35 years old; or
(xviii) the T cells are from a single healthy donor having a body mass index (BMI) less than 30 kg/m².

279. A composition comprising a population of T cells, wherein the T cells are from a healthy donor aged 18 to 35 years old and having a body mass index (BMI) less than 30 kg/m², and wherein the composition has one or more, two or more, three or more, four or more, five or more, six or more, seven or more, eight or more, nine or more, ten or more, eleven or more, twelve or more, thirteen or more, fourteen or more, fifteen or more, or all the following attributes:
(i) at least about 70% of the T cells are viable;
(ii) at least about 10% of total alleles have edited TRAC loci;
(iii) at least about 10% of total alleles have edited B2M loci;
(iv) at least about 10% of total alleles have a nucleic acid encoding HLA-E(A) knocked-in at the B2M locus, such as a nucleic acid comprising the sequence set forth in SEQ ID NO: 86, or a nucleic acid encoding HLA-E(A) having an amino acid sequence set forth in SEQ ID NO: 81 or SEQ ID NO: 132;
(v) at least about 10% of total alleles have a nucleic acid encoding CD19 CAR knocked-in at the TRAC locus, such as a nucleic acid comprising the sequence set forth in SEQ ID NO: 136, or a nucleic acid encoding a CD19 CAR having an amino acid sequence set forth in SEQ ID NO: 78 or SEQ ID NO: 138;
(vi) at least about 90% of the T cells are CD2+CD5+;
(vii) at least about 50% of the T cells are CD2+CD5+ and express CD19 CAR, such as the CD19 CAR having an amino acid sequence set forth in SEQ ID NO: 78 or SEQ ID NO: 138;
(viii) at least about 7,500,000 viable CD2+CD5+CD19 CAR+ cells per mL of the composition;
(ix) no detected bacterial growth;
(x) less than about 5 EU/mL endotoxin;
(xi) no detected mycoplasma;
(xii) less than about 70,000 TCR+ cells/kg;
(xiii) no cytokine-independent growth;
(xiv) no significant unexpected karyotype
(xv) less than about 5% of total alleles have translocation between TRAC and B2M; or
(xvi) negative for the presence of HIV-1, HIV-2, HTLV-1, HTLV-2, HAV, HBV, HCV, CMV, EBV, HHV6, HHV7, HHV8, and B19.

280. A composition comprising a population of T cells, wherein the T cells are from a healthy donor aged 18 to 35 years old and having a body mass index (BMI) less than 30 kg/m², and wherein the composition has one or more, two or more, three or more, four or more, five or more, six or more, seven or more, eight or more, nine or more, ten or more, eleven or more, twelve or more, thirteen or more, fourteen or more, fifteen or more, or all the following attributes:
(i) at least about 70% of the T cells are viable;
(ii) at least about 10% of total alleles have edited TRAC loci;
(iii) at least about 10% of total alleles have edited B2M loci;
(iv) at least about 10% of total alleles have a nucleic acid encoding HLA-E(A) knocked-in at the B2M locus, such as a nucleic acid comprising the sequence set forth in SEQ ID NO: 86, or a nucleic acid encoding HLA-E(A) having an amino acid sequence set forth in SEQ ID NO: 81 or SEQ ID NO: 132;
(v) at least about 10% of total alleles have a nucleic acid encoding CD19 CAR knocked-in at the TRAC locus, such as a nucleic acid comprising the sequence set forth in SEQ ID NO: 136, or a nucleic acid encoding a CD19 CAR having an amino acid sequence set forth in SEQ ID NO: 78 or SEQ ID NO: 138;
(vi) at least about 90% of the T cells are CD2+CD5+;
(vii) at least about 50% of the T cells are CD2+CD5+ and express CD19 CAR, such as the CD19 CAR having an amino acid sequence set forth in SEQ ID NO: 78 or SEQ ID NO: 138;
(viii) at least about 7,500,000 viable CD2+CD5+CD19 CAR+ cells per mL of the composition;
(ix) no detected bacterial growth;
(x) less than about 5 EU/mL endotoxin;
(xi) no detected mycoplasma;
(xii) less than about 70,000 TCR+ cells/kg;
(xiii) no cytokine-independent growth;
(xiv) no significant unexpected karyotype
(xv) less than about 5% of total alleles have translocation between TRAC and B2M; or
(xvi) negative for the presence of HIV-1, HIV-2, HTLV-1, HTLV-2, HAV, HBV, HCV, CMV, EBV, HHV6, HHV7, HHV8, and B19, and wherein the composition has one or more of the following attributes:
(a) less than about 0.1%, less than about 0.2%, less than about 0.3%, less than about 0.4%, less than about 0.5%, less than about 0.6%, less than about 0.7%, less than about 0.8%, less than about 0.9%, or less than about 1% of the T cells are TCR+ T cells;
(b) less than about 1 ng, less than about 2 ng, less than about 3 ng, less than about 4 ng, less than about 5 ng, less than about 6 ng, less than about 7 ng, less than about 8 ng, less than about 9 ng, less than about 10 ng, less than about 11 ng, less than about 12 ng, less than about 13 ng, less than about 14 ng, less than about 15 ng, less than about 16 ng, less than about 17 ng, less than about 18 ng, less than about 19 ng, or less than about 20 ng of Cas9 or Cas12a per mL of the composition;
(c) less than about 0.01 fg, less than about 0.02 fg, less than about 0.03 fg, less than about 0.04 fg, less than about 0.05 fg, less than about 0.06 fg, less than about 0.07 fg, less than about 0.08 fg, less than about 0.09 fg, or less than about 1.0 fg of Cas9 or Cas12a per T cell;
(d) less than about 1×10¹⁰, less than about 5×10¹⁰, less than about 1×10¹¹, less than about 5×10¹¹, or less than about 1×10¹² AAV6 capsids per mL of the composition;
(e) ten or fewer, nine or fewer, eight or fewer, seven or fewer, six or fewer, five or fewer, four or fewer, three or fewer, two or fewer, one or fewer, or no verified off-target indel sites at a genomic locus other than TRAC and B2M;
(f) less than about 0.5% of total alleles, less than about 1.0% of total alleles, less than about 1.5% of total alleles, less than about 2.0% of total alleles, less than about 2.5% of total alleles, less than about 3.0% of total alleles, less than about 3.5% of total alleles, or less than about 4.0% of total alleles have AAV6 integration at TRAC locus;
(g) less than about 0.5% of total alleles, less than about 0.6% of total alleles, less than about 0.7% of total alleles, less than about 0.8% of total alleles, less than about 0.9% of total alleles, less than about 1.0% of total alleles, less than about 1.1% of total alleles, less than about 1.2% of total alleles, less than about 1.3% of total alleles, less than about 1.4% of total alleles, less than about 1.5% of total alleles, less than about 1.6% of total alleles, less than about 1.7% of total alleles, less than about 1.8% of total alleles, less than about 1.9% of total alleles, or less than about 2.0% of total alleles have AAV6 integration at B2M locus;
(h) undetectable AAV6 integration at loci other than TRAC and B2M; or
(i) less than about 100 ng, less than about 150 ng, less than about 200 ng, less than about 250 ng, less than about 300 ng, less than about 350 ng, less than about 400 ng, less than about 450 ng, less than about 500 ng, less than about 550 ng, less than about 600 ng, less than about 650 ng, less than about 700 ng, less than about 750 ng, less than about 800 ng, less than about 850 ng, less than about 900 ng, less than about 950 ng, less than about 1000 ng, less than about 1100 ng, less than about 1200 ng, less than about 1300 ng, less than about 1400 ng, or less than about 1500 ng of Strep-Tactin Multimer activation reagent per mL of the composition.

281. A composition comprising a population of T cells, wherein the T cells are from a healthy donor aged 18 to 35 years old and having a body mass index (BMI) less than 30 kg/m², and wherein the composition has one or more, two or more, three or more, four or more, five or more, six or more, seven or more, eight or more, nine or more, ten or more, eleven or more, twelve or more, thirteen or more, fourteen or more, fifteen or more, or all the following attributes:
(i) at least about 70% of the T cells are viable;
(ii) at least about 10% of total alleles have edited TRAC loci;
(iii) at least about 10% of total alleles have edited B2M loci;
(iv) at least about 10% of total alleles have a nucleic acid encoding HLA-E(A) knocked-in at the B2M locus, such as a nucleic acid comprising the sequence set forth in SEQ ID NO: 86, or a nucleic acid encoding HLA-E(A) having an amino acid sequence set forth in SEQ ID NO: 81 or SEQ ID NO: 132;
(v) at least about 10% of total alleles have a nucleic acid encoding CD19 CAR knocked-in at the TRAC locus, such as a nucleic acid comprising the sequence set forth in SEQ ID NO: 136, or a nucleic acid encoding a CD19 CAR having an amino acid sequence set forth in SEQ ID NO: 78 or SEQ ID NO: 138;
(vi) at least about 90% of the T cells are CD2+CD5+;
(vii) at least about 50% of the T cells are CD2+CD5+ and express CD19 CAR, such as the CD19 CAR having an amino acid sequence set forth in SEQ ID NO: 78 or SEQ ID NO: 138;
(viii) at least about 7,500,000 viable CD2+CD5+CD19 CAR+ cells per mL of the composition;
(ix) no detected bacterial growth;
(x) less than about 5 EU/mL endotoxin;
(xi) no detected mycoplasma;
(xii) less than about 70,000 TCR+ cells/kg;
(xiii) no cytokine-independent growth;
(xiv) no significant unexpected karyotype
(xv) less than about 5% of total alleles have translocation between TRAC and B2M; or
(xvi) negative for the presence of HIV-1, HIV-2, HTLV-1, HTLV-2, HAV, HBV, HCV, CMV, EBV, HHV6, HHV7, HHV8, and B19, and wherein the composition has one or more of the following attributes:
(a) less than about 0.5% of the T cells are TCR+ T cells;
(b) less than about 2 ng of Cas9 or Cas12a per mL of the composition;
(c) less than about 0.05 fg of Cas9 or Cas12a per T cell;
(d) less than about 5×10¹¹ AAV6 capsids per mL of the composition;
(e) no verified off-target indel sites at a genomic locus other than TRAC and B2M;
(f) less than about 2.0% of total alleles have AAV6 integration at TRAC locus;
(g) less than about 1.0% of total alleles have AAV6 integration at B2M locus;
(h) undetectable AAV6 integration at loci other than TRAC and B2M; or
(i) less than about 900 ng of Strep-Tactin Multimer activation reagent per mL of the composition.

282. A composition comprising a population of T cells, wherein the composition has one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, or all the following attributes:
(i) at least about 70% of the T cells are viable determined by fluorescent microscopy;
(ii) at least about 10% of total alleles have edited TRAC loci determined by ddPCR;
(iii) at least about 10% of total alleles have edited B2M loci determined by ddPCR;
(iv) at least about 10% of total alleles have a nucleic acid encoding HLA-E(A) knocked-in at the B2M locus, such as a nucleic acid comprising the sequence set forth in SEQ ID NO: 86, or a nucleic acid encoding HLA-E(A) having an amino acid sequence set forth in SEQ ID NO: 81 or SEQ ID NO: 132, determined by ddPCR;
(v) at least about 10% of total alleles have a nucleic acid encoding CD19 CAR knocked-in at the TRAC locus, such as a nucleic acid comprising the sequence set forth in SEQ ID NO: 136, or a nucleic acid encoding a CD19 CAR having an amino acid sequence set forth in SEQ ID NO: 78 or SEQ ID NO: 138, determined by ddPCR;
(vi) at least about 90% of the T cells are CD2+CD5+ determined by flow cytometry;
(vii) at least about 50% of the T cells are CD2+CD5+ and express CD19 CAR, such as the CD19 CAR having an amino acid sequence set forth in SEQ ID NO: 78 or SEQ ID NO: 138, determined by flow cytometry;
(viii) at least about 7,500,000 viable CD2+CD5+CD19 CAR+ cells per mL of the composition determined by flow cytometry;
(ix) no detected bacterial growth determined by BacT/ALERT 3D;
(x) less than about 5 EU/mL endotoxin determined by Limulus Amoebocyte Lysate (LAL);
(xi) no detected mycoplasma determined by qPCR;
(xii) less than about 70,000 TCR+ cells/kg determined by flow cytometry and calculated by multiplying the % TCR+ cells by viable cell number to get % TCR cells/mL, then multiplying the % TCR cells/mL by volume to obtain total number of TCR+ cells, then dividing the total number TCR+ cells by 60 kg patient weight;
(xiii) no cytokine-independent growth between day 31 and day 70 in a cell-based assay having a limit of detection (LOD) of about 1.5E5 cells/mL;
(xiv) no significant unexpected karyotype determined by microscopy, wherein a significant unexpected karyotype is the same specific aberration or aberrant ploidy in more than 6 cells and occurs in two out of three test replicates or three out of three test replicates;
(xv) less than about 5% of total alleles have translocation between TRAC and B2M determined by ddPCR;
(xvi) negative for the presence of HIV-1, HIV-2, HTLV-1, HTLV-2, HAV, HBV, HCV, CMV, EBV, HHV6, HHV7, HHV8, and B19 determined by PCR;
(xvii) the T cells are from a healthy donor aged 18 to 35 years old; or
(xviii) the T cells are from a single healthy donor having a body mass index (BMI) less than 30 kg/m².

283. A composition comprising a population of T cells, wherein the T cells are from a healthy donor aged 18 to 35 years old and having a body mass index (BMI) less than 30 kg/m², and wherein the composition has one or more, two or more, three or more, four or more, five or more, six or more, seven or more, eight or more, nine or more, ten or more, eleven or more, twelve or more, thirteen or more, fourteen or more, fifteen or more, or all the following attributes:
(i) at least about 70% of the T cells are viable determined by fluorescent microscopy;
(ii) at least about 10% of total alleles have edited TRAC loci determined by ddPCR;
(iii) at least about 10% of total alleles have edited B2M loci determined by ddPCR;
(iv) at least about 10% of total alleles have a nucleic acid encoding HLA-E(A) knocked-in at the B2M locus, such as a nucleic acid comprising the sequence set forth in SEQ ID NO: 86, or a nucleic acid encoding HLA-E(A) having an amino acid sequence set forth in SEQ ID NO: 81 or SEQ ID NO: 132, determined by ddPCR;
(v) at least about 10% of total alleles have a nucleic acid encoding CD19 CAR knocked-in at the TRAC locus, such as a nucleic acid comprising the sequence set forth in SEQ ID NO: 136, or a nucleic acid encoding a CD19 CAR having an amino acid sequence set forth in SEQ ID NO: 78 or SEQ ID NO: 138, determined by ddPCR;
(vi) at least about 90% of the T cells are CD2+CD5+ determined by flow cytometry;
(vii) at least about 50% of the T cells are CD2+CD5+ and express CD19 CAR, such as the CD19 CAR having an amino acid sequence set forth in SEQ ID NO: 78 or SEQ ID NO: 138, determined by flow cytometry;
(viii) at least about 7,500,000 viable CD2+CD5+CD19 CAR+ cells per mL of the composition determined by flow cytometry;
(ix) no detected bacterial growth determined by BacT/ALERT 3D;
(x) less than about 5 EU/mL endotoxin determined by Limulus Amoebocyte Lysate (LAL);
(xi) no detected mycoplasma determined by qPCR;
(xii) less than about 70,000 TCR+ cells/kg determined by flow cytometry and calculated by multiplying the % TCR+ cells by viable cell number to get % TCR cells/mL, then multiplying the % TCR cells/mL by volume to obtain total number of TCR+ cells, then dividing the total number TCR+ cells by 60 kg patient weight;
(xiii) no cytokine-independent growth between day 31 and day 70 in a cell-based assay having a limit of detection (LOD) of about 1.5E5 cells/mL;
(xiv) no significant unexpected karyotype determined by microscopy, wherein a significant unexpected karyotype is the same specific aberration or aberrant ploidy in more than 6 cells and occurs in two out of three test replicates or three out of three test replicates;
(xv) less than about 5% of total alleles have translocation between TRAC and B2M determined by ddPCR; or
(xvi) negative for the presence of HIV-1, HIV-2, HTLV-1, HTLV-2, HAV, HBV, HCV, CMV, EBV, HHV6, HHV7, HHV8, and B19 determined by PCR.

284. A composition comprising a population of T cells, wherein the T cells are from a healthy donor aged 18 to 35 years old and having a body mass index (BMI) less than 30 kg/m², and wherein the composition has one or more, two or more, three or more, four or more, five or more, six or more, seven or more, eight or more, nine or more, ten or more, eleven or more, twelve or more, thirteen or more, fourteen or more, fifteen or more, or all the following attributes:
(i) at least about 70% of the T cells are viable determined by fluorescent microscopy;
(ii) at least about 10% of total alleles have edited TRAC loci determined by ddPCR;
(iii) at least about 10% of total alleles have edited B2M loci determined by ddPCR;
(iv) at least about 10% of total alleles have a nucleic acid encoding HLA-E(A) knocked-in at the B2M locus, such as a nucleic acid comprising the sequence set forth in SEQ ID NO: 86, or a nucleic acid encoding HLA-E(A) having an amino acid sequence set forth in SEQ ID NO: 81 or SEQ ID NO: 132, determined by ddPCR;
(v) at least about 10% of total alleles have a nucleic acid encoding CD19 CAR knocked-in at the TRAC locus, such as a nucleic acid comprising the sequence set forth in SEQ ID NO: 136, or a nucleic acid encoding a CD19 CAR having an amino acid sequence set forth in SEQ ID NO: 78 or SEQ ID NO: 138, determined by ddPCR;
(vi) at least about 90% of the T cells are CD2+CD5+ determined by flow cytometry;
(vii) at least about 50% of the T cells are CD2+CD5+ and express CD19 CAR, such as the CD19 CAR having an amino acid sequence set forth in SEQ ID NO: 78 or SEQ ID NO: 138, determined by flow cytometry;
(viii) at least about 7,500,000 viable CD2+CD5+CD19 CAR+ cells per mL of the composition determined by flow cytometry;
(ix) no detected bacterial growth determined by BacT/ALERT 3D;
(x) less than about 5 EU/mL endotoxin determined by Limulus Amoebocyte Lysate (LAL);
(xi) no detected mycoplasma determined by qPCR;
(xii) less than about 70,000 TCR+ cells/kg determined by flow cytometry and calculated by multiplying the % TCR+ cells by viable cell number to get % TCR cells/mL, then multiplying the % TCR cells/mL by volume to obtain total number of TCR+ cells, then dividing the total number TCR+ cells by 60 kg patient weight;
(xiii) no cytokine-independent growth between day 31 and day 70 in a cell-based assay having a limit of detection (LOD) of about 1.5E5 cells/mL;
(xiv) no significant unexpected karyotype determined by microscopy, wherein a significant unexpected karyotype is the same specific aberration or aberrant ploidy in more than 6 cells and occurs in two out of three test replicates or three out of three test replicates;
(xv) less than about 5% of total alleles have translocation between TRAC and B2M determined by ddPCR; or
(xvi) negative for the presence of HIV-1, HIV-2, HTLV-1, HTLV-2, HAV, HBV, HCV, CMV, EBV, HHV6, HHV7, HHV8, and B19 determined by PCR, and wherein the composition has one or more of the following attributes:
(a) less than about 0.1%, less than about 0.2%, less than about 0.3%, less than about 0.4%, less than about 0.5%, less than about 0.6%, less than about 0.7%, less than about 0.8%, less than about 0.9%, or less than about 1% of the T cells are TCR+ T cells;
(b) less than about 1 ng, less than about 2 ng, less than about 3 ng, less than about 4 ng, less than about 5 ng, less than about 6 ng, less than about 7 ng, less than about 8 ng, less than about 9 ng, less than about 10 ng, less than about 11 ng, less than about 12 ng, less than about 13 ng, less than about 14 ng, less than about 15 ng, less than about 16 ng, less than about 17 ng, less than about 18 ng, less than about 19 ng, or less than about 20 ng of Cas9 or Cas12a per mL of the composition;
(c) less than about 0.01 fg, less than about 0.02 fg, less than about 0.03 fg, less than about

0.04 fg, less than about 0.05 fg, less than about 0.06 fg, less than about 0.07 fg, less than about 0.08 fg, less than about 0.09 fg, or less than about 1.0 fg of Cas9 or Cas12a per T cell;
(d) less than about 1×10¹⁰, less than about 5×10¹⁰, less than about 1×10¹¹, less than about 5×10¹¹, or less than about 1×10¹² AAV6 capsids per mL of the composition;
(e) ten or fewer, nine or fewer, eight or fewer, seven or fewer, six or fewer, five or fewer, four or fewer, three or fewer, two or fewer, one or fewer, or no verified off-target indel sites at a genomic locus other than TRAC and B2M;
(f) less than about 0.5% of total alleles, less than about 1.0% of total alleles, less than about 1.5% of total alleles, less than about 2.0% of total alleles, less than about 2.5% of total alleles, less than about 3.0% of total alleles, less than about 3.5% of total alleles, or less than about 4.0% of total alleles have AAV6 integration at TRAC locus;
(g) less than about 0.5% of total alleles, less than about 0.6% of total alleles, less than about 0.7% of total alleles, less than about 0.8% of total alleles, less than about 0.9% of total alleles, less than about 1.0% of total alleles, less than about 1.1% of total alleles, less than about 1.2% of total alleles, less than about 1.3% of total alleles, less than about 1.4% of total alleles, less than about 1.5% of total alleles, less than about 1.6% of total alleles, less than about 1.7% of total alleles, less than about 1.8% of total alleles, less than about 1.9% of total alleles, or less than about 2.0% of total alleles have AAV6 integration at B2M locus;
(h) undetectable AAV6 integration at loci other than TRAC and B2M; or
(i) less than about 100 ng, less than about 150 ng, less than about 200 ng, less than about 250 ng, less than about 300 ng, less than about 350 ng, less than about 400 ng, less than about 450 ng, less than about 500 ng, less than about 550 ng, less than about 600 ng, less than about 650 ng, less than about 700 ng, less than about 750 ng, less than about 800 ng, less than about 850 ng, less than about 900 ng, less than about 950 ng, less than about 1000 ng, less than about 1100 ng, less than about 1200 ng, less than about 1300 ng, less than about 1400 ng, or less than about 1500 ng of Strep-Tactin Multimer activation reagent per mL of the composition.

285. A composition comprising a population of T cells, wherein the T cells are from a healthy donor aged 18 to 35 years old and having a body mass index (BMI) less than 30 kg/m², and wherein the composition has one or more, two or more, three or more, four or more, five or more, six or more, seven or more, eight or more, nine or more, ten or more, eleven or more, twelve or more, thirteen or more, fourteen or more, fifteen or more, or all the following attributes:
(i) at least about 70% of the T cells are viable determined by fluorescent microscopy;
(ii) at least about 10% of total alleles have edited TRAC loci determined by ddPCR;
(iii) at least about 10% of total alleles have edited B2M loci determined by ddPCR;
(iv) at least about 10% of total alleles have a nucleic acid encoding HLA-E(A) knocked-in at the B2M locus, such as a nucleic acid comprising the sequence set forth in SEQ ID NO: 86, or a nucleic acid encoding HLA-E(A) having an amino acid sequence set forth in SEQ ID NO: 81 or SEQ ID NO: 132, determined by ddPCR;
(v) at least about 10% of total alleles have a nucleic acid encoding CD19 CAR knocked-in at the TRAC locus, such as a nucleic acid comprising the sequence set forth in SEQ ID NO: 136, or a nucleic acid encoding a CD19 CAR having an amino acid sequence set forth in SEQ ID NO: 78 or SEQ ID NO: 138, determined by ddPCR;
(vi) at least about 90% of the T cells are CD2+CD5+ determined by flow cytometry;
(vii) at least about 50% of the T cells are CD2+CD5+ and express CD19 CAR, such as the CD19 CAR having an amino acid sequence set forth in SEQ ID NO: 78 or SEQ ID NO: 138, determined by flow cytometry;
(viii) at least about 7,500,000 viable CD2+CD5+CD19 CAR+ cells per mL of the composition determined by flow cytometry;
(ix) no detected bacterial growth determined by BacT/ALERT 3D;
(x) less than about 5 EU/mL endotoxin determined by Limulus Amoebocyte Lysate (LAL);
(xi) no detected mycoplasma determined by qPCR;
(xii) less than about 70,000 TCR+ cells/kg determined by flow cytometry and calculated by multiplying the % TCR+ cells by viable cell number to get % TCR cells/mL, then multiplying the % TCR cells/mL by volume to obtain total number of TCR+ cells, then dividing the total number TCR+ cells by 60 kg patient weight;
(xiii) no cytokine-independent growth between day 31 and day 70 in a cell-based assay having a limit of detection (LOD) of about 1.5E5 cells/mL;
(xiv) no significant unexpected karyotype determined by microscopy, wherein a significant unexpected karyotype is the same specific aberration or aberrant ploidy in more than 6 cells and occurs in two out of three test replicates or three out of three test replicates;
(xv) less than about 5% of total alleles have translocation between TRAC and B2M determined by ddPCR; or
(xvi) negative for the presence of HIV-1, HIV-2, HTLV-1, HTLV-2, HAV, HBV, HCV, CMV, EBV, HHV6, HHV7, HHV8, and B19 determined by PCR, and wherein the composition has one or more of the following attributes:
(a) less than about 0.5% of the T cells are TCR+ T cells;
(b) less than about 2 ng of Cas9 or Cas12a per mL of the composition;
(c) less than about 0.05 fg of Cas9 or Cas12a per T cell;
(d) less than about 5×10¹¹ AAV6 capsids per mL of the composition;
(e) no verified off-target indel sites at a genomic locus other than TRAC and B2M;
(f) less than about 2.0% of total alleles have AAV6 integration at TRAC locus;
(g) less than about 1.0% of total alleles have AAV6 integration at B2M locus;
(h) undetectable AAV6 integration at loci other than TRAC and B2M; or
(i) less than about 900 ng of Strep-Tactin Multimer activation reagent per mL of the composition.

286. The composition of any of embodiments 278-285, wherein the T cells (i) are from a single healthy donor aged 18 to 35 years old and having a body mass index (BMI) less than 30 kg/m², and (ii) comprise (a) a modified TRAC locus comprising a transgene sequence comprising the sequence set forth in SEQ ID NO: 94, and (b) a modified B2M locus comprising a transgene sequence comprising the sequence set forth in SEQ ID NO: 137.

287. The composition of any of embodiments 278-285, wherein the T cells (i) are from a single healthy donor aged 18 to 35 years old and having a body mass index (BMI) less than 30 kg/m², and (ii) comprise (a) a modified TRAC locus comprising a transgene sequence comprising the sequence set forth in SEQ ID NO: 136, and (b) a modified B2M locus comprising a transgene sequence comprising the sequence set forth in SEQ ID NO: 86.

288. The composition of any of embodiments 258-287, wherein the attributes have been determined or detected by a method comprising:
(i) determining the % T cell viability by image cytometry cell counting;
(ii) determining the % T cells are CD2+CD5+ by flow cytometry;
(iii) determining the % T cells expressing CD19 CAR by flow cytometry;
(iv) determining the % T cells are HLA-I+ by flow cytometry;
(v) determining the ratio of % TCR+ to % CAR+ T cells by flow cytometry;
(vi) determining the % of total alleles having translocation between TRAC and B2M by polymerase chain reaction (PCR) or digital droplet PCR (ddPCR);
(vii) determining the number of viable CD2+CD5+CD19 CAR+ cells per mL of the composition by flow cytometry and/or image cytometry;
(viii) determining the % of total alleles having TRAC loci edited by ddPCR;
(ix) determining the % TCR+ T cells by flow cytometry;
(x) determining the % of total alleles having B2M loci edited by ddPCR;
(xi) determining the % of alleles having a nucleic acid encoding HLA-E(A), such as the HLA-E(A) having the amino acid sequence set forth in SEQ ID NO: 81 or SEQ ID NO: 132, knocked-in at B2M locus by ddPCR;
(xii) determining the % of alleles having a nucleic acid encoding CD19 CAR, such as a nucleic acid comprising the sequence set forth in SEQ ID NO: 136, or a nucleic acid encoding a CD19 CAR having an amino acid sequence set forth in SEQ ID NO: 78 or SEQ ID NO: 138, knocked-in at the TRAC locus by ddPCR;
(xiii) determining the amount of Cas9 or Cas12a per mL of the composition by ELISA;
(xiv) determining the amount of Cas9 or Cas12a per cell by dividing (the amount of Cas9 or Cas12a per mL of the composition by ELISA (numerator)) by (the number of cells per mL of the composition (denominator));
(xv) determining the amount of AAV6 capsids per mL of the composition by ELISA;
(xvi) determining the number of verified off-target indel sites at a genomic locus other than TRAC and B2M by next-generation sequencing (NGS);
(xvii) determining the % of total alleles having AAV6 integration at TRAC locus by ddPCR;
(xviii) determining the % of total alleles having AAV6 integration at B2M locus by ddPCR;
(xix) determining AAV6 integration at loci other than TRAC and B2M by NGS, such as a NGS-based method that identifies the genomic location of insertions of AAV6 inverted terminal repeats (ITRs);
(xx) determining the amount of Strep-Tactin Multimer activation reagent per mL of composition by ELISA;
(xxi) detecting bacterial growth by measuring the amount of CO₂ present in media;
(xxii) determining the amount of endotoxin by compendial testing, such as by the Limulus Amebocyte Lysate (LAL) Test;
(xxiii) detecting mycoplasma by PCR;
(xxiv) detecting cytokine-independent growth by a cell-based assay, such as by culturing cells in cytokine-free media and determining cell concentration and timepoints;
(xxv) detecting a significant unexpected karyotype by microscopy, such as by staining chromosomes and assessing them microscopically using G-banding technique; or
(xxvi) detecting HIV, HTLV, HAV, HBV, HCV, CMV, EBV, HHV6, HHV7, HHV8, and B19 by PCR, such as by RT-qPCR.

289. The composition of any of embodiments 233-288, wherein the T cells have the following attributes:
(i) at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% of the T cells are CD4+CD45RA+CCR7+;
(ii) less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, less than about 75%, less than about 80%, less than about 85%, less than about 90%, less than about 95%, or less than about 99% of the T cells are CD8+CD45RA-CCR7−;
(iii) less than about 1%, less than about 2%, less than about 3%, less than about 4%, less than about 5%, less than about 6%, less than about 7%, less than about 8%, less than about 9%, less than about 10%, less than about 11%, less than about 12%, less than about 13%, less than about 14%, less than about 15%, less than about 16%, less than about 17%, less than about 18%, less than about 19%, less than about 20%, less than about 21%, less than about 22%, less than about 23%, less than about 24%, or less than about 25% of the T cells are CD3+aCAS3+;
(iv) at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% of the T cells are CD8+CCR7+; and
(v) at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% of the T cells are CD4+CD27+CD28+. 290. The composition of any of embodiments 233-288, wherein the T cells have the following attributes:
(i) at least about 13% to at least about 80% of the T cells are CD4+CD45RA+CCR7+;
(ii) less than about 3% to less than about 90% of the T cells are CD8+CD45RA-CCR7−;
(iii) less than about 1% to less than about 15% of the T cells are CD3+aCAS3+;
(iv) at least about 2% to at least about 93% of the T cells are CD8+CCR7+; and
(v) at least about 55% to at least about 98% of the T cells are CD4+CD27+CD28+. 291. The composition of any of embodiments 233-288, wherein the T cells have the following attributes:
(i) at least about 10% of the T cells are CD4+CD45RA+CCR7+;
(ii) less than about 25% of the T cells are CD8+CD45RA-CCR7−;
(iii) less than about 3.7% of the T cells are CD3+aCAS3+;
(iv) at least about 50% of the T cells are CD8+CCR7+; and
(v) at least about 89% of the T cells are CD4+CD27+CD28+. 292. The composition of any of embodiments 289-291, wherein the percent of CD4+CD45RA+CCR7+, CD8+CD45RA-CCR7−, CD3+aCAS3+, CD8+CCR7+, or CD4+CD27+CD28+ T cells is determined by flow cytometry.

293. The composition of any of embodiments 233-292, wherein the composition comprises about 25×10⁶, about 30×10⁶, about 35×10⁶, about 40×10⁶, about 45×10⁶, about 50×10⁶, about 55×10⁶, about 60×10⁶, 65×10⁶, about 70×10⁶, about 75×10⁶, about 100×10⁶, about 150×10⁶, about 200×10⁶, about 250×10⁶, about 300×10⁶, about 350×10⁶, about 400×10⁶, about 450×10⁶, about 500×10⁶, about 550×10⁶, or about 600×10⁶ of the T cells per mL of the composition.

294. A genetically modified T cell from the composition of any of embodiments 233-293.

295. A method of treatment, the method comprising administering the cell of any one of embodiments 1-98 and 294 or the composition of any one of embodiments 233-293 to a subject having a disease or disorder.

296. The method of embodiment 295, wherein the disease or disorder is associated with an antigen targeted by the CAR.

297. The method of embodiment 296, wherein the antigen is CD19.

298. The method of any of embodiments 295-297, wherein the disease or disorder is an autoimmune disease.

299. The method of embodiment 298, wherein the autoimmune disease is systemic lupus erythematosus (SLE), idiopathic inflammatory myopathies (IIM), multiple sclerosis (MS), systemic sclerosis (SSc), or rheumatoid arthritis (RA).

300. The method of any of embodiments 295-297, wherein the disease or disorder is cancer.

301. The method of embodiment 300, wherein the cancer is a lymphoma or a leukemia.

302. The method of embodiment 300, wherein the cancer is a lymphoma that is a large B cell lymphoma.

303. The method of embodiment 301, wherein the lymphoma is a non-Hodgkin lymphoma.

304. The method of any of embodiments 295-303, wherein about 25×10⁶′ about 30×10⁶, about 35×10⁶, about 40×10⁶, about 45×10⁶, about 50×10⁶, about 55×10⁶, about 60×10⁶, 65×10⁶, about 70×10⁶, about 75×10⁶, about 80×10⁶, about 85×10⁶, about 90×10⁶, about 95×10⁶, about 100×10⁶, about 125×10⁶, about 150×10⁶, about 175×10⁶, about 200×10⁶, about 225×10⁶, about 250×10⁶, about 275×10⁶, about 300×10⁶, about 325×10⁶, about 350×10⁶, about 375×10⁶, about 400×10⁶, about 425×10⁶, about 450×10⁶, about 475×10⁶, about 500×10⁶, about 525×10⁶, about 550×10⁶, about 575×10⁶, or about 600×10⁶ of the T cells are administered to the subject.

305. The method of any of embodiments 295-304, wherein less than about 5×10⁴ TCRαβ+ T cells/kg patient weight, less than about 6×10⁴ TCRαβ+ T cells/kg patient weight, or less than about 7×10⁴ TCRαβ+ T cells/kg patient weight are administered to the subject.

### X. FURTHER EXEMPLARY EMBODIMENTS

1. A genetically engineered T cell comprising:
(a) a genetic disruption in the endogenous TRAC gene, wherein the genetic disruption in the endogenous TRAC gene is in a target site in exon 1 of the TRAC gene, and wherein the target site in exon 1 of the TRAC gene has the sequence set forth in SEQ ID NO: 84, a contiguous portion thereof of at least 12 nucleotides (nt), or a complementary sequence of the foregoing;
(b) a transgene encoding a CD19 chimeric antigen receptor (CAR) comprising the amino acid sequence set forth in SEQ ID NO: 78, wherein the transgene encoding the CD19 CAR is integrated at the target site in exon 1 of the TRAC gene;
(c) a genetic disruption in the endogenous B-2 microglobulin (B2M) gene; wherein the genetic disruption in the endogenous B2M gene is in a target site in exon 2 of the B2M gene, and wherein the target site in exon 2 of the B2M gene has the sequence set forth in SEQ ID NO: 85, a contiguous portion thereof of at least 12 nucleotides (nt), or a complementary sequence of the foregoing; and
(d) a transgene encoding a single chain HLA-E fusion protein comprising the amino acid sequence set forth in SEQ ID NO: 81, wherein the transgene encoding the single chain HLA-E fusion protein is integrated at the target site in exon 2 of the B2M gene.

2. The genetically engineered T cell of embodiment 1, wherein one or more alleles of the endogenous TRAC gene are disrupted.

3. The genetically engineered T cell of embodiment 1 or 2, wherein all alleles of the endogenous TRAC gene are disrupted.

4. The genetically engineered T cell of any of embodiments 1-3, wherein the genetically engineered T cell has reduced protein expression of TCR alpha chain encoded from the endogenous TRAC gene, optionally wherein the genetically engineered T cell has reduced protein expression of the TCR alpha chain on the surface of the T cell, more optionally wherein the genetically engineered T cell does not express detectable TCR alpha chain.

5. The genetically engineered T cell of any of embodiments 1-4, wherein the genetically engineered T cell has reduced expression of CD3 on the cell surface, optionally wherein the genetically engineered T cell does not express detectable CD3 on the cell surface.

6. The genetically engineered T cell of any of embodiments 1-5, wherein one or more alleles of the endogenous B2M gene are disrupted.

7. The genetically engineered T cell of any of embodiments 1-6, wherein all alleles of the endogenous B2M gene are disrupted.

8. The genetically engineered T cell of any of embodiments 1-7, wherein the genetically engineered T cell has reduced protein expression of B2M encoded from the endogenous B2M gene, optionally wherein the genetically engineered T cell does not express detectable B2M.

9. The genetically engineered T cell of any of embodiments 1-8, wherein the genetically engineered T cell has reduced expression of one or more HLA class I molecules (e.g., HLA-A class I, HLA-B class I and/or HLA-C class I) on the cell surface, optionally wherein the genetically engineered T cell has no detectable expression of one or more HLA class I molecules (e.g., HLA-A class I, HLA-B class I and/or HLA-C class I) on the cell surface.

10. The genetically engineered T cell of any of embodiments 1-9, wherein the genetically engineered T cell has no detectable expression of HLA-A class I, HLA-B class I and HLA-C class I on the cell surface.

11. The genetically engineered T cell of any of embodiments 1-10, wherein the single chain HLA-E fusion protein is capable of engaging inhibitory receptors on the surface of NK cells.

12. The genetically engineered T cell of any of embodiments 1-11, wherein the transgene encoding the CD19 CAR is present in the disrupted TRAC gene in the T cell under the operable control of a promoter.

13. The genetically engineered T cell of embodiment 12, wherein the promoter is a heterologous promoter of the TRAC gene.

14. The genetically engineered T cell of embodiment 13, wherein the heterologous promoter is or comprises a human elongation factor 1 alpha (EF1α) promoter or a variant thereof.

15. The genetically engineered T cell of any of embodiments 1-14, wherein the transgene encoding the CD19 CAR comprises the sequence set forth in SEQ ID NO: 136.

16. The genetically engineered T cell of any of embodiments 1-15, wherein the transgene encoding the single chain HLA-E fusion protein comprises the sequence set forth in SEQ ID NO: 86.

17. The genetically engineered T cell of any of embodiments 1-16, wherein the transgene encoding the CD19 CAR comprises the sequence set forth in SEQ ID NO: 136 and the transgene encoding the single chain HLA-E fusion protein comprises the sequence set forth in SEQ ID NO: 86.

18. The genetically engineered T cell of any of embodiments 1-17, wherein the T cell is a primary T cell.

19. The genetically engineered T cell of embodiment 18, wherein the primary T cell is from a human donor.

20. The genetically engineered T cell of embodiment 19, wherein the human donor is a healthy donor aged 18 to 35 years old and having a body mass index (BMI) less than 30 kg/m².

21. The genetically engineered T cell of embodiment 20, wherein the healthy donor is male.

22. The genetically engineered T cell of embodiment 20, wherein the healthy donor is female.

23. The genetically engineered T cell of embodiment 22, wherein the female is nulliparous and non-pregnant.

24. A method of producing a genetically engineered T cell, the method comprising:
(a) introducing into a T cell a first CRISPR-Cas system comprising a Cas protein and a guide RNA (gRNA) for inducing a genetic disruption at a target site in exon 1 of an endogenous T cell receptor alpha constant (TRAC) gene; wherein the target site in exon 1 of the endogenous TRAC gene has the sequence set forth in SEQ ID NO: 84, a contiguous portion thereof of at least 12 nucleotides (nt), or a complementary sequence of the foregoing, and the gRNA comprises a spacer sequence that is complementary to the target site;
(b) introducing into the T cell a second CRISPR-Cas system comprising a Cas protein and a guide RNA (gRNA) for inducing a genetic disruption at a target site in exon 2 of an endogenous B-2 microglobulin (B2M) gene, wherein the target site in exon 2 of the B2M gene has the sequence set forth in SEQ ID NO: 85, a contiguous portion thereof of at least 12 nucleotides (nt), or a complementary sequence of the foregoing, and the gRNA comprises a spacer sequence that is complementary to the target site;
(c) introducing into the T cell a polynucleotide comprising a transgene encoding a CD19 chimeric antigen receptor (CAR) comprising the amino acid sequence set forth in SEQ ID NO: 78; and
(d) introducing into the T cell a polynucleotide comprising a transgene encoding a single chain HLA-E fusion protein comprising the amino acid sequence set forth in SEQ ID NO: 81.

25. The method of embodiment 24, wherein each CRISPR-Cas system is a ribonucleoprotein (RNP) complex comprising the Cas protein and the gRNA.

26. The method of embodiment 25, wherein the first CRISPR-Cas system is a ribonucleoprotein (RNP) complex comprising a Cas9 protein and the gRNA.

27. The method of any of embodiments 24-26, wherein the Cas is a S. pyogenes Cas9 (spCas9).

28. The method of any of embodiments 24-27, wherein the spacer sequence of the gRNA complementary to the target site in exon 1 of the endogenous TRAC gene comprises the nucleic acid sequence of SEQ ID NO: 87, or a contiguous portion thereof of at least 12 nt.

29. The method of any of embodiments 24-28, wherein introducing the first CRISPR-Cas system disrupts one or more alleles of the endogenous TRAC gene.

30. The method of any of embodiments 24-29, wherein introducing the first CRISPR-Cas system disrupts all alleles of the endogenous TRAC gene.

31. The method of any of embodiments 24-30, wherein introducing the first CRISPR-Cas system into the T cell reduces protein expression of TCR alpha chain encoded from the endogenous TRAC gene, optionally protein expression of the TCR alpha chain on the surface of the T cell, more optionally wherein there is no detectable expression of TCR alpha chain in the T cell.

32. The method of any of embodiments 24-31, wherein the second CRISPR-Cas system is a ribonucleoprotein (RNP) complex comprising a Cas12a protein and the gRNA.

33. The method of embodiment 32, wherein the Cas12a is Francisella novicida Cas12a (FnCas12a), Lachnospiraceae bacterium Cas12a (LbCas12a), Acidaminococcus sp. Cas12a (AsCas12a).

34. The method of any of embodiments 24-33, wherein the spacer sequence of the gRNA complementary to the target site in exon 2 of the endogenous B2M gene comprises the nucleic acid sequence of SEQ ID NO: 105, or a contiguous portion thereof of at least 12 nt.

35. The method of any of embodiments 24-34, wherein introducing the second CRISPR-Cas system disrupts one or more alleles of the endogenous B2M gene.

36. The method of any of embodiments 24-34, wherein introducing the second CRISPR-Cas system disrupts all alleles of the endogenous B2M gene.

37. The method of any of embodiments 24-36, wherein introducing the second CRISPR-Cas system reduces protein expression of B2M encoded from the endogenous B2M gene, optionally wherein there is no detectable expression of B2M in the T cell.

38. The method of any of embodiments 24-37, wherein introducing the second CRISPR-Cas system reduces expression of one or more HLA class I molecules (e.g., HLA-A class I, HLA-B class I and/or HLA-C class I) on the cell surface, optionally wherein there is no detectable expression of one or more HLA class I molecules (e.g., HLA-A class I, HLA-B class I and/or HLA-C class I) on the cell surface.

39. The method of any of embodiments 24-38, wherein introducing the second CRISPR-Cas system results in no detectable expression of HLA-A class I, HLA-B class I and HLA-C class I on the cell surface.

40. The method of any of embodiments 24-39, wherein each gRNA independently comprises a spacer sequence between 14 nt and 24 nt, or between 16 nt and 22 nt in length.

41. The method of any of embodiments 24-40, wherein each gRNA independently comprises a spacer sequence that is 18 nt, 19 nt, 20 nt, 21 nt, or 22 nt in length.

42. The method of any of embodiments 24-41, wherein each gRNA further comprises a scaffold sequence for binding the respective Cas protein.

43. The method of any of embodiments 24-42, wherein the gRNA is modified by one or more modified nucleotides, wherein the one or more modified nucleotides are for increased stability of the gRNA.

44. The method of any of embodiments 24-43, wherein the gRNA targeting the endogenous TRAC gene comprises the sequence set forth in SEQ ID NO: 82 or SEQ ID NO: 92.

45. The method of any of embodiments 24-44, wherein the gRNA targeting the endogenous B2M gene comprises the sequence set forth in SEQ ID NO: 83.

46. The method of any of embodiments 24-45, wherein the gRNA targeting the endogenous TRAC gene and/or the gRNA targeting the endogenous B2M gene induces a double strand break.

47. The method of any of embodiments 24-46, wherein the transgene encoding a single chain HLA-E fusion protein is integrated via homology directed repair (HDR) at the target site in the B2M gene.

48. The method of any of embodiments 24-47, wherein the polynucleotide comprising the transgene encoding the single chain HLA-E fusion protein further comprises one or more homology arm(s) linked to the transgene, wherein the one or more homology arm(s) comprise a sequence homologous to nucleic acid sequences surrounding the target site sequence in the endogenous B2M gene.

49. The method of embodiment 48, wherein the polynucleotide comprising the transgene encoding the single chain HLA-E fusion protein comprises the structure [5′ homology arm]-[transgene]-[3′ homology arm], wherein the 5′ homology arm and 3′ homology arm comprises nucleic acid sequences homologous to the nucleic acid sequences surrounding the target site sequence in the endogenous B2M gene.

50. The method of embodiment 48 or embodiment 49, wherein the 5′ homology arm and 3′ homology arm independently are at or about 200, 300, 400, 500, 600, 700 or 800 nucleotides in length, or any value between any of the foregoing.

51. The method of any of embodiments 48-50, wherein the 5′ homology arm comprises the sequence set forth in SEQ ID NO: 79 or a sequence that has at least at or about 90%, 91%, 92%, 93%, 94%, 95%, 96%, 97%, 98% or 99% sequence identity to SEQ ID NO: 79 or a partial sequence thereof, and/or the 3′ homology arm comprises the sequence set forth in SEQ ID NO: 80, a sequence that has at least at or about 90%, 91%, 92%, 93%, 94%, 95%, 96%, 97%, 98% or 99% sequence identity to SEQ ID NO: 80 or a partial sequence thereof.

52. The method of any of embodiments 48-51, wherein the 5′ homology arm comprises the sequence set forth in SEQ ID NO: 79 and the 3′ homology arm comprises the sequence set forth in SEQ ID NO: 80.

53. The method of any of embodiments 24-52, wherein the transgene encoding the CD19 CAR is integrated via homology directed repair (HDR) at the target site in the TRAC gene.

54. The method of any of embodiments 24-53, wherein the polynucleotide comprising the transgene encoding the CD19 CAR further comprises one or more homology arm(s) linked to the transgene, wherein the one or more homology arm(s) comprise a sequence homologous to nucleic acid sequences surrounding the target site sequence in the endogenous TRAC gene.

55. The method of embodiment 54, wherein the polynucleotide comprising the transgene encoding the CD19 CAR comprises the structure [5′ homology arm]-[transgene]-[3′ homology arm], wherein the 5′ homology arm and 3′ homology arm comprises nucleic acid sequences homologous to the nucleic acid sequences surrounding the target site sequence in the endogenous TRAC gene.

56. The method of embodiment 54 or embodiment 55, wherein the 5′ homology arm and 3′ homology arm independently are at or about 200, 300, 400, 500, 600, 700 or 800 nucleotides in length, or any value between any of the foregoing.

57. The method of any of embodiments 54-56, wherein the 5′ homology arm comprises the sequence set forth in SEQ ID NO: 76 or a sequence that has at least at or about 90%, 91%, 92%, 93%, 94%, 95%, 96%, 97%, 98% or 99% sequence identity to SEQ ID NO: 76 or a partial sequence thereof, and/or the 3′ homology arm comprises the sequence set forth in SEQ ID NO: 77, a sequence that has at least at or about 90%, 91%, 92%, 93%, 94%, 95%, 96%, 97%, 98% or 99% sequence identity to SEQ ID NO: 77 or a partial sequence thereof.

58. The method of any of embodiments 54-57, wherein the 5′ homology arm comprises the sequence set forth in SEQ ID NO: 76 and the 3′ homology arm comprises the sequence set forth in SEQ ID NO: 77.

59. The method of any of embodiments 47-58, wherein the transgene encoding the single chain HLA-E fusion is integrated to be under the operable control of the endogenous B2M promoter, optionally wherein the transgene encoding the single chain HLA-E fusion protein comprises one or more multicistronic element(s) positioned upstream of the nucleotide sequence encoding the single chain HLA-E fusion, more optionally wherein the one or more multicistronic element is or comprises aT2A, a P2A, an E2A, or an F2A element.

60. The method of any of embodiments 53-59, wherein the transgene encoding the CD19 CAR is operably linked to a heterologous promoter to control expression of the CD19 CAR.

61. The method of embodiment 60, wherein the heterologous promoter is or comprises a human elongation factor 1 alpha (EF1α) promoter or a variant thereof.

62. The method of any of embodiments 24-61, wherein:
the introducing of the polynucleotide comprising the transgene encoding the CD19 CAR is by transduction of a first viral vector comprising the polynucleotide encoding the CD19 CAR; and
the introducing of the polynucleotide comprising the transgene encoding the single chain HLA-E fusion protein is by transduction of a second viral vector comprising the polynucleotide comprising the transgene encoding the single chain HLA-E fusion protein.

63. The method of embodiment 62, wherein a mixture comprising the first viral vector and the second viral vector are introduced into the T cell.

64. The method of embodiment 62 or embodiment 63, wherein the first viral vector is an AAV vector and the second viral vector is an AAV vector, optionally wherein the AAV vector is an AAV6 vector.

65. The method of any of embodiments 24-64, wherein the first CRISPR-Cas system and the second CRISPR-Cas system are introduced into the T cell via electroporation.

66. The method of any of embodiments 24-65, wherein the polynucleotide comprising the transgene encoding the CD19 CAR comprises the nucleotide sequence set forth in SEQ ID NO: 94.

67. The method of any of embodiments 24-66, wherein the polynucleotide comprising the transgene encoding the single chain HLA-E fusion protein comprises the nucleotide sequence set forth in SEQ ID NO: 137.

68. The method of any of embodiments 24-67, wherein the polynucleotide comprising the transgene encoding the CD19 CAR comprises the nucleotide sequence set forth in SEQ ID NO: 94 and the polynucleotide comprising the transgene encoding the single chain HLA-E fusion protein comprises the nucleotide sequence set forth in SEQ ID NO: 137.

69. The method of any of embodiments 24-68, wherein the transgene encoding the CD19 CAR comprises the nucleotide sequence set forth in SEQ ID NO: 136.

70. The method of any of embodiments 24-69, wherein the transgene encoding the single chain HLA-E fusion protein comprises the nucleotide sequence set forth in SEQ ID NO: 86.

71. The method of any of embodiments 24-70, wherein the first CRISPR-Cas system and second CRISPR-Cas system are introduced simultaneously.

72. The method of any of embodiments 65-71, wherein after the electroporation, the method comprises introducing the polynucleotides by transducing the T cells with a mixture of viral vectors, wherein the mixture of viral vectors comprises a first viral vector comprising the polynucleotide comprising the transgene encoding the CD19 CAR and a second viral vector comprising the polynucleotide comprising the transgene encoding the single chain HLA-E fusion protein.

73. The method of embodiment 72, wherein the first viral vector is an AAV vector and the second viral vector is an AAV vector, optionally wherein the AAV vector is an AAV6 vector.

74. The method of embodiment 72 or embodiment 73, wherein transducing is within about 15 minutes, within about 30 minutes, within about 60 minutes, or within about 2 hours, after the electroporation.

75. The method of any of embodiments 72-74, wherein after the transducing the method further comprises incubating the cell under static conditions in serum free media for a period of time for recovery of the cells.

76. The method of any of embodiments 72-75, wherein the method further comprises expanding the T cells 2 to 8 doublings or 6 to 7 doublings, optionally expanding the T cells in the presence of one or more recombinant cytokines, optionally in the presence of one or more recombinant IL-2, IL-7 and/or IL-15.

77. The method of embodiment 76, wherein the expanding is carried out with perfusion.

78. The method of any of embodiments 24-77, wherein prior to each of the introducing, the method comprises stimulating the T cells with one or more stimulatory agent(s) under conditions to stimulate or activate the T cells, optionally wherein the one or more stimulatory agent(s) comprises anti-CD3 and/or anti-CD28 antibodies, optionally anti-CD3/anti-CD28 Fabs.

79. The method of any of embodiments 24-78, wherein the T cell is a primary T cell.

80. The method of embodiment 79, wherein the primary T cell is from a human donor.

81. The method of embodiment 80, wherein the human donor is a healthy donor aged 18 to 35 years old and having a body mass index (BMI) less than 30 kg/m².

82. The method of embodiment 81, wherein the healthy donor is male.

83. The method of embodiment 81, wherein the healthy donor is female.

84. The method of embodiment 83, wherein the female is nulliparous and non-pregnant.

85. The method of any of embodiments 24-84, wherein the method is performed ex vivo.

86. The method of any of embodiments 24-85, wherein the method is performed in vitro.

87. The method of any of embodiments 24-86, further comprising harvesting T cells produced by the method.

88. The method of embodiment 87, further comprising depleting CD3+ T cells from the harvested T cells.

89. The method of embodiment 87 or embodiment 88, further comprising formulating the harvested T cells with a cryoprotectant.

90. A genetically engineered T cell produced by the method of any of embodiments 24-89.

91. A composition comprising a population of genetically engineered T cells of any of embodiments 1-23.

92. A composition comprising a population of genetically engineered T cells produced by the method of any of embodiments 24-89.

93. The composition of embodiment 91 or embodiment 92, wherein the composition is a pharmaceutical composition comprising a pharmaceutically acceptable excipient.

94. The composition of any of embodiments 91-93, wherein the composition comprises a cyroprotectant, optionally wherein the cryoprotectant is DMSO.

95. The composition of any of embodiments 91-94, wherein the composition comprises CD4+ T cells and CD8+ T cells.

96. The composition of embodiment 95, wherein the ratio of CD4+ T cells to CD8+ T cells is from at or about 1:5 to at or about 5:1, optionally from at or about 1:3 to at or about 3:1.

97. The composition of any of embodiments 91-96, wherein at least about 50% of the T cells are viable.

98. The composition of any of embodiments 91-96, wherein at least about 60% of the T cells are viable.

99. The composition of any of embodiments 91-96, wherein at least about 70% of the T cells are viable.

100. The composition of any of embodiments 91-96, wherein at least about 80% of the T cells are viable.

101. The composition of any of embodiments 91-96, wherein at least about 90% of the T cells are viable.

102. The composition of any of embodiments 91-101, wherein at least about 10% of total alleles have edited TRAC loci.

103. The composition of any of embodiments 91-101, wherein at least about 20% of total alleles have edited TRAC loci.

104. The composition of any of embodiments 91-101, wherein at least about 30% of total alleles have edited TRAC loci.

105. The composition of any of embodiments 91-101, wherein at least about 40% of total alleles have edited TRAC loci.

106. The composition of any of embodiments 91-101, wherein at least about 50% of total alleles have edited TRAC loci.

107. The composition of any of embodiments 91-101, wherein at least about 60% of total alleles have edited TRAC loci.

108. The composition of any of embodiments 91-101, wherein at least about 70% of total alleles have edited TRAC loci.

109. The composition of any of embodiments 91-101, wherein at least about 80% of total alleles have edited TRAC loci.

110. The composition of any of embodiments 91-101, wherein at least about 90% of total alleles have edited TRAC loci.

111. The composition of any of embodiments 91-110, wherein at least about 10% of total alleles have edited B2M loci.

112. The composition of any of embodiments 91-110, wherein at least about 20% of total alleles have edited B2M loci.

113. The composition of any of embodiments 91-110, wherein at least about 30% of total alleles have edited B2M loci.

114. The composition of any of embodiments 91-110, wherein at least about 40% of total alleles have edited B2M loci.

115. The composition of any of embodiments 91-110, wherein at least about 50% of total alleles have edited B2M loci.

116. The composition of any of embodiments 91-110, wherein at least about 60% of total alleles have edited B2M loci.

117. The composition of any of embodiments 91-110, wherein at least about 70% of total alleles have edited B2M loci.

118. The composition of any of embodiments 91-110, wherein at least about 80% of total alleles have edited B2M loci.

119. The composition of any of embodiments 91-110, wherein at least about 90% of total alleles have edited B2M loci.

120. The composition of any of embodiments 91-119, wherein at least about 10% of total alleles have the transgene encoding the CD19 CAR integrated in the TRAC locus.

121. The composition of any of embodiments 91-119, wherein at least about 20% of total alleles have the transgene encoding the CD19 CAR integrated in the TRAC locus.

122. The composition of any of embodiments 91-119, wherein at least about 30% of total alleles have the transgene encoding the CD19 CAR integrated in the TRAC locus.

123. The composition of any of embodiments 91-119, wherein at least about 40% of total alleles have the transgene encoding the CD19 CAR integrated in the TRAC locus.

124. The composition of any of embodiments 91-119, wherein at least about 50% of total alleles have the transgene encoding the CD19 CAR integrated in the TRAC locus.

125. The composition of any of embodiments 91-119, wherein at least about 60% of total alleles have the transgene encoding the CD19 CAR integrated in the TRAC locus.

126. The composition of any of embodiments 91-119, wherein at least about 70% of total alleles have the transgene encoding the CD19 CAR integrated in the TRAC locus.

127. The composition of any of embodiments 91-119, wherein at least about 80% of total alleles have the transgene encoding the CD19 CAR integrated in the TRAC locus.

128. The composition of any of embodiments 91-119, wherein at least about 90% of total alleles have the transgene encoding the CD19 CAR integrated in the TRAC locus.

129. The composition of any of embodiments 91-128, wherein at least about 10% of total alleles have the transgene encoding the single chain HLA-E fusion protein integrated in the B2M locus.

130. The composition of any of embodiments 91-128, wherein at least about 20% of total alleles have the transgene encoding the single chain HLA-E fusion protein integrated in the B2M locus.

131. The composition of any of embodiments 91-128, wherein at least about 30% of total alleles have the transgene encoding the single chain HLA-E fusion protein integrated in the B2M locus.

132. The composition of any of embodiments 91-128, wherein at least about 40% of total alleles have the transgene encoding the single chain HLA-E fusion protein integrated in the B2M locus.

133. The composition of any of embodiments 91-128, wherein at least about 50% of total alleles have the transgene encoding the single chain HLA-E fusion protein integrated in the B2M locus.

134. The composition of any of embodiments 91-128, wherein at least about 60% of total alleles have the transgene encoding the single chain HLA-E fusion protein integrated in the B2M locus.

135. The composition of any of embodiments 91-128, wherein at least about 70% of total alleles have the transgene encoding the single chain HLA-E fusion protein integrated in the B2M locus.

136. The composition of any of embodiments 91-128, wherein at least about 80% of total alleles have the transgene encoding the single chain HLA-E fusion protein integrated in the B2M locus.

137. The composition of any of embodiments 91-128, wherein at least about 90% of total alleles have the transgene encoding the single chain HLA-E fusion protein integrated in the B2M locus.

138. The composition of any of embodiments 91-137, wherein at least about 50% of the T cells are CD2+CD5+.

139. The composition of any of embodiments 91-137, wherein at least about 60% of the T cells are CD2+CD5+.

140. The composition of any of embodiments 91-137, wherein at least about 70% of the T cells are CD2+CD5+.

141. The composition of any of embodiments 91-137, wherein at least about 80% of the T cells are CD2+CD5+.

142. The composition of any of embodiments 91-137, wherein at least about 90% of the T cells are CD2+CD5+.

143. The composition of any of embodiments 91-142, wherein at least about 10% of the T cells are CD2+CD5+ and express the CD19 CAR.

144. The composition of any of embodiments 91-142, wherein at least about 20% of the T cells are CD2+CD5+ and express the CD19 CAR.

145. The composition of any of embodiments 91-142, wherein at least about 30% of the T cells are CD2+CD5+ and express the CD19 CAR.

146. The composition of any of embodiments 91-142, wherein at least about 40% of the T cells are CD2+CD5+ and express the CD19 CAR.

147. The composition of any of embodiments 91-142, wherein at least about 50% of the T cells are CD2+CD5+ and express the CD19 CAR.

148. The composition of any of embodiments 91-142, wherein at least about 60% of the T cells are CD2+CD5+ and express the CD19 CAR.

149. The composition of any of embodiments 91-142, wherein at least about 70% of the T cells are CD2+CD5+ and express the CD19 CAR.

150. The composition of any of embodiments 91-142, wherein at least about 80% of the T cells are CD2+CD5+ and express the CD19 CAR.

151. The composition of any of embodiments 91-142, wherein at least about 90% of the T cells are CD2+CD5+ and express the CD19 CAR.

152. The composition of any of embodiments 91-151, wherein the composition comprises at least about 5,000,000 viable CD2+CD5+CD19 CAR+ cells per mL of the composition.

153. The composition of any of embodiments 91-151, wherein the composition comprises at least about 5,500,000 viable CD2+CD5+CD19 CAR+ cells per mL of the composition.

154. The composition of any of embodiments 91-151, wherein the composition comprises at least about 6,000,000 viable CD2+CD5+CD19 CAR+ cells per mL of the composition.

155. The composition of any of embodiments 91-151, wherein the composition comprises at least about 6,500,000 viable CD2+CD5+CD19 CAR+ cells per mL of the composition.

156. The composition of any of embodiments 91-151, wherein the composition comprises at least about 7,000,000 viable CD2+CD5+CD19 CAR+ cells per mL of the composition.

157. The composition of any of embodiments 91-151, wherein the composition comprises at least about 7,500,000 viable CD2+CD5+CD19 CAR+ cells per mL of the composition.

158. The composition of any of embodiments 91-151, wherein the composition comprises at least about 8,000,000 viable CD2+CD5+CD19 CAR+ cells per mL of the composition.

159. The composition of any of embodiments 91-151, wherein the composition comprises at least about 8,500,000 viable CD2+CD5+CD19 CAR+ cells per mL of the composition.

160. The composition of any of embodiments 91-151, wherein the composition comprises at least about 9,000,000 viable CD2+CD5+CD19 CAR+ cells per mL of the composition.

161. The composition of any of embodiments 91-151, wherein the composition comprises at least about 9,500,000 viable CD2+CD5+CD19 CAR+ cells per mL of the composition.

162. The composition of any of embodiments 91-151, wherein the composition comprises at least about 10,000,000 viable CD2+CD5+CD19 CAR+ cells per mL of the composition.

163. The composition of any of embodiments 91-151, wherein the composition comprises at least about 11,000,000 viable CD2+CD5+CD19 CAR+ cells per mL of the composition.

164. The composition of any of embodiments 91-151, wherein the composition comprises at least about 12,000,000 viable CD2+CD5+CD19 CAR+ cells per mL of the composition.

165. The composition of any of embodiments 91-151, wherein the composition comprises at least about 13,000,000 viable CD2+CD5+CD19 CAR+ cells per mL of the composition.

166. The composition of any of embodiments 91-151, wherein the composition comprises at least about 14,000,000 viable CD2+CD5+CD19 CAR+ cells per mL of the composition.

167. The composition of any of embodiments 91-151, wherein the composition comprises at least about 15,000,000 viable CD2+CD5+CD19 CAR+ cells per mL of the composition.

168. The composition of any of embodiments 91-151, wherein the composition comprises at least about 16,000,000 viable CD2+CD5+CD19 CAR+ cells per mL of the composition.

169. The composition of any of embodiments 91-151, wherein the composition comprises at least about 17,000,000 viable CD2+CD5+CD19 CAR+ cells per mL of the composition.

170. The composition of any of embodiments 91-151, wherein the composition comprises at least about 18,000,000 viable CD2+CD5+CD19 CAR+ cells per mL of the composition.

171. The composition of any of embodiments 91-151, wherein the composition comprises at least about 19,000,000 viable CD2+CD5+CD19 CAR+ cells per mL of the composition.

172. The composition of any of embodiments 91-151, wherein the composition comprises at least about 20,000,000 viable CD2+CD5+CD19 CAR+ cells per mL of the composition.

173. The composition of any of embodiments 91-172, wherein the composition has less than about 1 EU/mL endotoxin.

174. The composition of any of embodiments 91-172, wherein the composition has less than about 2 EU/mL endotoxin.

175. The composition of any of embodiments 91-172, wherein the composition has less than about 3 EU/mL endotoxin.

176. The composition of any of embodiments 91-172, wherein the composition has less than about 4 EU/mL endotoxin.

177. The composition of any of embodiments 91-172, wherein the composition has less than about 5 EU/mL endotoxin.

178. The composition of any of embodiments 91-172, wherein the composition has less than about 6 EU/mL endotoxin.

179. The composition of any of embodiments 91-172, wherein the composition has less than about 7 EU/mL endotoxin.

180. The composition of any of embodiments 91-172, wherein the composition has less than about 8 EU/mL endotoxin.

181. The composition of any of embodiments 91-172, wherein the composition has less than about 9 EU/mL endotoxin.

182. The composition of any of embodiments 91-172, wherein the composition has less than about 10 EU/mL endotoxin.

183. The composition of any of embodiments 91-172, wherein the composition has less than about 11 EU/mL endotoxin.

184. The composition of any of embodiments 91-172, wherein the composition has less than about 12 EU/mL endotoxin.

185. The composition of any of embodiments 91-172, wherein the composition has less than about 13 EU/mL endotoxin.

186. The composition of any of embodiments 91-172, wherein the composition has less than about 14 EU/mL endotoxin.

187. The composition of any of embodiments 91-172, wherein the composition has less than about 15 EU/mL endotoxin.

188. The composition of any of embodiments 91-187, wherein the composition has less than about 100,000 TCR+ cells/kg patient weight.

189. The composition of any of embodiments 91-187, wherein the composition has less than about 90,000 TCR+ cells/kg patient weight.

190. The composition of any of embodiments 91-187, wherein the composition has less than about 80,000 TCR+ cells/kg patient weight.

191. The composition of any of embodiments 91-187, wherein the composition has less than about 70,000 TCR+ cells/kg patient weight.

192. The composition of any of embodiments 91-187, wherein the composition has less than about 60,000 TCR+ cells/kg patient weight.

193. The composition of any of embodiments 91-187, wherein the composition has less than about 50,000 TCR+ cells/kg patient weight.

194. The composition of any of embodiments 91-187, wherein the composition has less than about 40,000 TCR+ cells/kg patient weight.

195. The composition of any of embodiments 91-194, wherein less than about 10% of total alleles have translocation between TRAC and B2M.

196. The composition of any of embodiments 91-194, wherein less than about 9% of total alleles have translocation between TRAC and B2M.

197. The composition of any of embodiments 91-194, wherein less than about 8% of total alleles have translocation between TRAC and B2M.

198. The composition of any of embodiments 91-194, wherein less than about 7% of total alleles have translocation between TRAC and B2M.

199. The composition of any of embodiments 91-194, wherein less than about 6% of total alleles have translocation between TRAC and B2M.

200. The composition of any of embodiments 91-194, wherein less than about 5% of total alleles have translocation between TRAC and B2M.

201. The composition of any of embodiments 91-194, wherein less than about 4% of total alleles have translocation between TRAC and B2M.

202. The composition of any of embodiments 91-194, wherein less than about 3% of total alleles have translocation between TRAC and B2M.

203. The composition of any of embodiments 91-194, wherein less than about 2% of total alleles have translocation between TRAC and B2M.

204. The composition of any of embodiments 91-194, wherein less than about 1% of total alleles have translocation between TRAC and B2M.

205. The composition of any of embodiments 91-204, wherein the composition has no detected bacterial growth.

206. The composition of any of embodiments 91-205, wherein the composition has no detected mycoplasma.

207. The composition of any of embodiments 91-206, wherein the composition has no cytokine-independent growth.

208. The composition of any of embodiments 91-207, wherein the composition has no significant unexpected karyotype.

209. The composition of any of embodiments 91-208, wherein the composition is negative for the presence of HIV-1, HIV-2, HTLV-1, HTLV-2, HAV, HBV, HCV, CMV, EBV, HHV6, HHV7, HHV8, and/or B19.

210. The composition of any of embodiments 97-209, wherein the percentage of T cells that are viable is determined by determined by fluorescent microscopy.

211. The composition of any of embodiments 102-210, wherein the percentage of total alleles that have edited TRAC loci is determined by ddPCR.

212. The composition of any of embodiment 111-211, wherein the percentage of total alleles that have edited B2M loci is determined by ddPCR.

213. The composition of any of embodiment 120-212, wherein the percentage of total alleles that have a transgene encoding the CD19 CAR integrated in the TRAC locus is determined by ddPCR.

214. The composition of any of embodiment 129-213, wherein the percentage of total alleles that have a transgene encoding the single chain HLA-E fusion protein integrated in the B2M locus is determined by ddPCR.

215. The composition of any of embodiment 138-214, wherein the percentage of T cells that are CD2+CD5+ is determined by flow cytometry.

216. The composition of any of embodiment 143-215, wherein the percentage of T cells that are CD2+CD5+ and express the CD19 CAR is determined by flow cytometry.

217. The composition of any of embodiment 152-216, wherein the number of viable CD2+CD5+CD19 CAR+ cells per mL of the composition is determined by flow cytometry.

218. The composition of any of embodiment 173-217, wherein the endotoxin level is determined by Limulus Amoebocyte Lysate (LAL).

219. The composition of any of embodiment 188-218, wherein the number of TCR+ cells/kg is determined by flow cytometry and calculated by multiplying the % TCR+ cells by viable cell number to get % TCR cells/mL, then multiplying the % TCR cells/mL by volume to obtain total number of TCR+ cells, then dividing the total number TCR+ cells by 60 kg patient weight.

220. The composition of any of embodiment 195-219, wherein the percentage of total alleles that have translocation between TRAC and B2M is determined by ddPCR.

221. The composition of any of embodiment 205-220, wherein the detection of bacterial growth is determined by BacT/ALERT 3D.

222. The composition of any of embodiment 206-221, wherein the detection of mycoplasma is determined by qPCR.

223. The composition of any of embodiment 207-222, wherein the no cytokine independent growth is determined between day 31 and day 70 in a cell-based assay having a limit of detection (LOD) of about 1.5E5 cells/mL.

224. The composition of any of embodiment 208-223, wherein the no significant unexpected karyotype is determined by microscopy, wherein a significant unexpected karyotype is the same specific aberration or aberrant ploidy in more than 6 cells and occurs in two out of three test replicates or three out of three test replicates.

225. The composition of any of embodiment 209-224, wherein the presence of HIV-1, HIV-2, HTLV-1, HTLV-2, HAV, HBV, HCV, CMV, EBV, HHV6, HHV7, HHV8, and/or B19 is determined by PCR.

226. A composition comprising a population of T cells, wherein the T cells are from a healthy donor aged 18 to 35 years old and having a body mass index (BMI) less than 30 kg/m², and wherein the composition has the following attributes:
(i) at least about 70% of the T cells are viable;
(ii) at least about 10% of total alleles have edited TRAC loci;
(iii) at least about 10% of total alleles have edited B2M loci;
(iv) at least about 10% of total alleles have a transgene encoding a single chain HLA-E fusion protein integrated in the B2M locus, such as a transgene comprising the sequence set forth in SEQ ID NO: 86, or a transgene encoding a single chain HLA-E fusion protein having an amino acid sequence set forth in SEQ ID NO: 81;
(v) at least about 10% of total alleles have a transgene encoding a CD19 CAR integrated in the TRAC locus, such as a transgene comprising the sequence set forth in SEQ ID NO: 136 or a transgene encoding a CD19 CAR having an amino acid sequence set forth in SEQ ID NO: 78;
(vi) at least about 90% of the T cells are CD2+CD5+;
(vii) at least about 50% of the T cells are CD2+CD5+ and express the CD19 CAR, such as the CD19 CAR having an amino acid sequence set forth in SEQ ID NO: 78 or SEQ ID NO: 138;
(viii) at least about 7,500,000 viable CD2+CD5+CD19 CAR+ cells per mL of the composition;
(ix) no detected bacterial growth;
(x) less than about 5 EU/mL endotoxin;
(xi) no detected mycoplasma;
(xii) less than about 70,000 TCR+ cells/kg;
(xiii) no cytokine-independent growth;
(xiv) no significant unexpected karyotype
(xv) less than about 5% of total alleles have translocation between TRAC and B2M; and
(xvi) negative for the presence of HIV-1, HIV-2, HTLV-1, HTLV-2, HAV, HBV, HCV, CMV, EBV, HHV6, HHV7, HHV8, and/or B19.

227. A composition comprising a population of T cells, wherein the T cells are from a healthy donor aged 18 to 35 years old and having a body mass index (BMI) less than 30 kg/m², and wherein the composition has the following attributes:
(i) at least about 70% of the T cells are viable determined by fluorescent microscopy;
(ii) at least about 10% of total alleles have edited TRAC loci determined by ddPCR;
(iii) at least about 10% of total alleles have edited B2M loci determined by ddPCR;
(iv) at least about 10% of total alleles have a transgene encoding a single chain HLA-E fusion protein integrated in the B2M locus, such as a transgene comprising the sequence set forth in SEQ ID NO: 86, or a transgene encoding a single chain HLA-E fusion protein having an amino acid sequence set forth in SEQ ID NO: 81, determined by ddPCR;
(v) at least about 10% of total alleles have a transgene encoding a CD19 CAR integrated in the TRAC locus, such as a transgene comprising the sequence set forth in SEQ ID NO: 136 or a transgene encoding a CD19 CAR having an amino acid sequence set forth in SEQ ID NO: 78, determined by ddPCR;
(vi) at least about 90% of the T cells are CD2+CD5+ determined by flow cytometry;
(vii) at least about 50% of the T cells are CD2+CD5+ and express CD19 CAR, such as the CD19 CAR having an amino acid sequence set forth in SEQ ID NO: 78 or SEQ ID NO: 138, determined by flow cytometry;
(viii) at least about 7,500,000 viable CD2+CD5+CD19 CAR+ cells per mL of the composition determined by flow cytometry;
(ix) no detected bacterial growth determined by BacT/ALERT 3D;
(x) less than about 5 EU/mL endotoxin determined by Limulus Amoebocyte Lysate (LAL);
(xi) no detected mycoplasma determined by qPCR;
(xii) less than about 70,000 TCR+ cells/kg determined by flow cytometry and calculated by multiplying the % TCR+ cells by viable cell number to get % TCR cells/mL, then multiplying the % TCR cells/mL by volume to obtain total number of TCR+ cells, then dividing the total number TCR+ cells by 60 kg patient weight;
(xiii) no cytokine-independent growth between day 31 and day 70 in a cell-based assay having a limit of detection (LOD) of about 1.5E5 cells/mL;
(xiv) no significant unexpected karyotype determined by microscopy, wherein a significant unexpected karyotype is the same specific aberration or aberrant ploidy in more than 6 cells and occurs in two out of three test replicates or three out of three test replicates;
(xv) less than about 5% of total alleles have translocation between TRAC and B2M determined by ddPCR; and
(xvi) negative for the presence of HIV-1, HIV-2, HTLV-1, HTLV-2, HAV, HBV, HCV, CMV, EBV, HHV6, HHV7, HHV8, and/or B19 determined by PCR.

228. The composition of any of embodiments 91-227, wherein the composition comprises about 25×10⁶, about 30×10⁶, about 35×10⁶, about 40×10⁶, about 45×10⁶, about 50×10⁶, about 55×10⁶, about 60×10⁶, 65×10⁶, about 70×10⁶, about 75×10⁶, about 100×10⁶, about 150×10⁶, about 200×10⁶, about 250×10⁶, about 300×10⁶, about 350×10⁶, about 400×10⁶, about 450×10⁶, about 500×10⁶, about 550×10⁶, or about 600×10⁶ of the T cells per mL of the composition.

229. A method of treatment, the method comprising administering the T cell of any one of embodiments 1-23 or the composition of any one of embodiments 91-228 to a subject having a disease or disorder associated with CD19.

230. The method of embodiment 229, wherein the disease or disorder is an autoimmune disease.

231. The method of embodiment 230, wherein the autoimmune disease is systemic lupus erythematosus (SLE), idiopathic inflammatory myopathies (IIM), multiple sclerosis (MS), systemic sclerosis (SSc), or rheumatoid arthritis (RA).

232. The method of any of embodiments 229-231, wherein about 25×10⁶′ about 30×10⁶, about 35×10⁶, about 40×10⁶, about 45×10⁶, about 50×10⁶, about 55×10⁶, about 60×10⁶, 65×10⁶, about 70×10⁶, about 75×10⁶, about 80×10⁶, about 85×10⁶, about 90×10⁶, about 95×10⁶, about 100×10⁶, about 125×10⁶, about 150×10⁶, about 175×10⁶, about 200×10⁶, about 225×10⁶, about 250×10⁶, about 275×10⁶, about 300×10⁶, about 325×10⁶, about 350×10⁶, about 375×10⁶, about 400×10⁶, about 425×10⁶, about 450×10⁶, about 475×10⁶, about 500×10⁶, about 525×10⁶, about 550×10⁶, about 575×10⁶, or about 600×10⁶ of the T cells are administered to the subject.

233. The method of any of embodiments 229-232, wherein less than about 5×10⁴ TCRαβ+ T cells/kg patient weight, less than about 6×10⁴ TCRαβ+ T cells/kg patient weight, or less than about 7×10⁴ TCRαβ+ T cells/kg patient weight are administered to the subject.

### XI. EXAMPLES

The following examples are included for illustrative purposes only and are not intended to limit the scope of the invention.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

### Example 1 Assessment of the Relationship Between Healthy Donor Ame and Body Mass Index on T Cell Differentiation

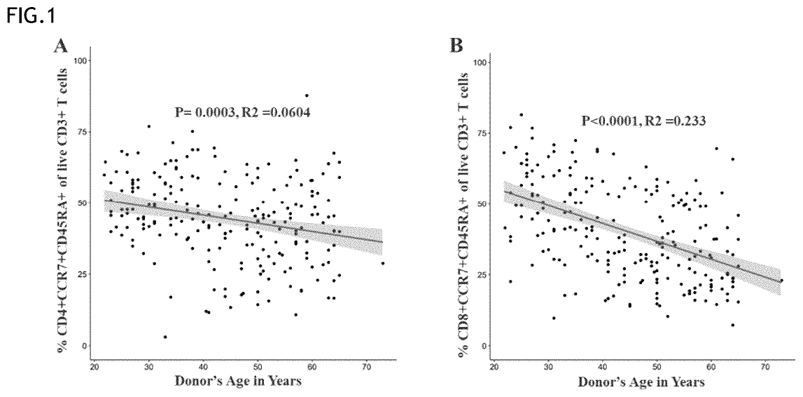

216 unique healthy donors from a range of ages were assessed for a relationship between age and frequencies of naive CD4+ and CD8+ T cells in apheresis material. As shown in FIG. 1, donors' age holds significant relationships with the frequencies of naïve CD4+ and CD8+ T cells in apheresis material. P values were derived by performing a t test of the simple linear regression slope. Donors' age showed inverse correlative relationship with the frequencies of less differentiated T cells in starting materials.

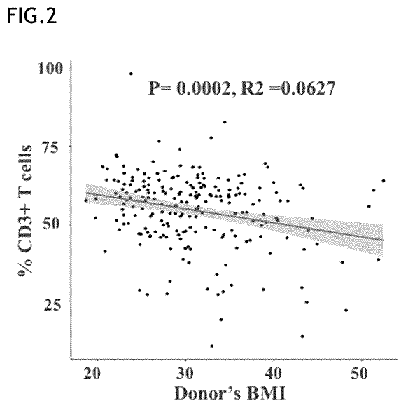

214 unique healthy donors having a range of body mass index (BMI) were assessed for a relationship between BMI and frequencies of CD3+ T cells in donors' apheresis materials. As shown in FIG. 2, a higher BMI holds negative relationship with the frequencies of CD3+ T cells in donors' apheresis materials.

### Example 2 Generation and Assessment of Healthy Donor Allogeneic CD19 CAR T Cells

Primary human T cells from healthy donors between the age of 18 to 35 years old and having a body mass index (BMI) less than 30 kg/m² were engineered to produce an allogeneic T cell composition by a strategy to reduce graft vs. Host Disease (GvHD), host-versus-graft (HvG) rejections, and lack-of-self recognition and NK cell-mediated rejection.

Mononuclear cells from healthy donors between the age of 18 to 35 years old and having a body mass index (BMI) less than 30 kg/m² were collected via leukapheresis, washed, concentrated, and cryopreserved for engineering starting material.

The engineering strategy used clustered regularly interspaced short palindromic repeats (CRISPR) technology to knock out (KO) T-cell receptor alpha constant (TRAC) and beta-2 microglobulin (B2M) loci to abrogate T-cell receptor (TCR) and human leukocyte antigen (HLA) class I expression, respectively, in order to mitigate graft versus host and host versus graft interactions; and introducing a histocompatibility antigen alpha chain E (HLA-E)-based molecule by knock-in (KI) at the B2M locus to reduce natural killer (NK) cell mediated rejection. The sequence encoding a CAR targeting CD19 was knocked in (KI) at the TRAC locus. Both the CAR and the HLA-E molecules were delivered using recombinant adeno-associated viral vector serotype 6 (rAAV6 vector)-mediated homology directed recombination (HDR).

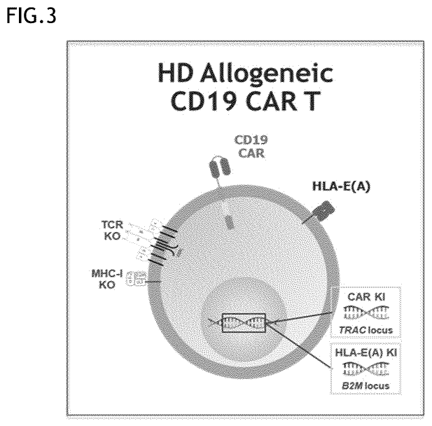

Table E1 and FIG. 3 provide a summary of the allogeneic strategy.

TABLE E1
Allogeneic T Cell Functional Edits

| Locus Edit | Purpose | Function |
|---|---|---|
| KO TCR alpha constant | Knock-out TRAC locus disrupting | Prevent GvHD |
| (TRAC) | endogenous TCR expression | |
| KI CD19 CAR at TRAC locus | Express CD19 CAR | Target B-cell associated |
| | | disease |
| KO Beta 2 microglobulin | Knock-out B2M locus eliminating HLA | Limit rejection of CAR T |
| (B2M) | Class I expression | by host T cells (HvG |
| | | reaction) |
| KI HLA-E in B2M locus | Express HLA-E NK cell inhibitory | Limit rejection of CAR T |
| | ligand | by host NK cells (HvG |
| | | reaction) |

Abbreviations: B2M, beta 2 microglobulin; CAR, chimeric antigen receptor; GvHD, graft versus host disease; HDR, homology-directed repair; HLA, human leukocyte antigen; HLA-E, HLA class I histocompatibility antigen, alpha chain E; HvG, host versus graft; KI, knock-in, KO, knock-out; NK, natural killer; TCR, T-cell receptor; TRAC, TCR alpha constant.

a. Genomic Editing Strategy and Generation of Healthy Donor Allogeneic Engineered T cell Product

A systematic and comprehensive approach was used to select and characterize the specificity of the sgRNAs used to knock-out (KO) the TRAC and B2M loci. Candidate gRNA target sites were selected according to the protospacer adjacent motif (PAM) sequence for the respective Cas. Each gRNA further comprised a scaffold sequence for the respective Cas and included base modifications.

CRISPR-associated protein (Cas)9-based TRAC gRNA-A (SEQ ID NO: 82) was selected for the disruption of the target site sequence GAGAATCAAAATCGGTGAAT (SEQ ID NO: 84) at the TRAC locus. The gRNA included the spacer sequence GAGAAUCAAAAUCGGUGAAU SEQ ID NO: 87, a scaffold sequence (SEQ ID NO: 88) for S, pyogenes Cas9 (spCas9) and included certain base modifications.

Cas12a-based B2M gRNA-B (SEQ ID NO: 83) was selected for disruption of the target site sequence AGTGGGGGTGAATTCAGTGTA (SEQ ID NO: 85) at the B2M locus. The gRNA included a spacer sequence and a Cas12a binding scaffold sequence set forth in SEQ ID NO: 89 added to the 5′-terminus of the spacer targeting domain, and was further modified with a DNA extension set forth in SEQ ID NO: 90 and base modifications.

The cryopreserved leukapheresis starting material described above was positively selected for CD4 and CD8 positive T cells using a CliniMACS® Plus selection instrument in which a magnetic field positively selected the bead-bound CD8+ T cells from the mixture, followed by positively selected CD4+ T cells using CD4 selection beads. The CD4+ and CD8+ selected fractions were mixed to a 1:1 ratio and activated by addition of Expamer soluble Fab CD3/CD28 reagent, composed of the Fab agents on a streptavidin mutein backbone (Strep-Tactin Multimer) (see e.g., PCT publication No. WO2018/197949) at 37° C. in media. Within about 48 hours, D-Biotin was added to reversibly dissociate the Fab reagents from the backbone to disrupt the stimulation, and the stimulatory reagent was removed by washing the cells.

For gene editing, the activated T cells were electroporated with ribonucleoprotein (RNP) complexes containing the respective nuclease and the gRNA. Specifically, for introducing a genetic disruption at the endogenous TRAC locus, the cells were electroporated with RNP complexes containing Streptococcus pyogenes Cas9 and TRAC-targeting gRNA set forth in SEQ ID NO: 82. For introducing a genetic disruption at the endogenous B2M locus, the cells were electroporated with RNP complexes containing Cas12a and B2M-targeting gRNA set forth in SEQ ID NO: 83. The total concentration of RNPs in the electroporation solution was 1.7 to 2.5 μM RNP complex containing the TRAC-targeting gRNA and 0.0625 to 0.5 μM RNP complex containing the B2M-targeting gRNA.

After electroporation, a mixture of rAAV6 preparations each containing donor templates either for targeted integration (KI) of transgene sequences for the CAR at the TRAC locus or for HLA-E at the B2M locus were added to the cells in the presence of a DNA-PK inhibitor. The general structure of the donor templates were as follows: [5′ homology arm]-[transgene sequences]-[3′ homology arm].

Specifically, the donor template for KI of the CAR into the TRAC locus included 600 base pair nucleic acid sequences homologous to sequences surrounding the target integration site in exon 1 of the human TRAC gene (5′ homology arm sequence set forth in SEQ ID NO: 76; 3′ homology arm sequence set forth in SEQ ID NO: 77) and the CAR transgene nucleic acid sequence set forth in SEQ ID NO: 136 encoding the CD19 CAR set forth in SEQ ID NO: 78 (SEQ ID NO: 138 without the signal peptide). The transgene sequence encoding the CAR also included a heterologous EF1a-HTLV1R promoter (SEQ ID NO: 91) operably connected to the nucleotide sequence encoding the CAR to control expression of the CAR when expressed from a cell introduced with the donor template by HDR KI into the disrupted TRAC locus.

The donor template for KI of an HLA-E fusion protein into the B2M locus included ˜800 base pair nucleic acid sequences homologous to sequences surrounding the target integration site of the human B2M gene (5′ homology arm sequence set forth in SEQ ID NO: 79; 3′ homology arm sequence set forth in SEQ ID NO: 80) and an HLA-E fusion transgene nucleic acid sequence set forth in SEQ ID NO: 86 encoding the HLA-E fusion construct set forth in SEQ ID NO: 81. The HLA-E fusion protein transgene sequence is inserted in the RNP site in exon2 of B2M in frame with the endogenous amino acid sequence and driven by the endogenous B2M promoter. The HLA-E fusion protein contains a 5′ T2A cleavable sequence to remove endogenous B2M sequence amino acids. The remaining HLA-E fusion protein sequence encodes a single chain fusion protein with HLA-E specific binding peptide and B2M covalently attached to HLA-E through linkers. The encoded HLA-E fusion protein contained a B2M leader sequence (amino acids 1-20 of SEQ ID NO: 81), a specific HLA-E binding peptide (amino acids 21-29 of SEQ ID NO: 81), a (G4S)x3 linker (amino acids 30 to 44 of SEQ ID NO: 81), B2M coding sequence without signal peptide (amino acids 45 to 143 of SEQ ID NO: 81), (G4S)x4 linker (SEQ ID NO: 144 to 163 of SEQ ID NO:81), and HLA-E coding sequence without signal peptide (amino acids 164 to 500 of SEQ ID NO:81). When expressed, the B2M leader is cleaved from the fusion protein.

Following electroporation and AAV transduction, cells were incubated for recovery under static conditions before expansion in serum-free media. Expansion was carried out using a Xuri Cell Expansion System W25 (Xuri) with perfusion. The cells were expanded between 6 to 7 cumulative fold doublings.

Once harvest criteria of 6 to 7 cumulative fold doublings was achieved, the harvested material was sampled, and cell counting was performed to calculate the total number of T cells prior to CD3+ depletion. The cells were then contacted with anti-CD3 depletion reagent beads to label residual T cells expressing surface TCRαβ complex. Cells with beads bound were removed using CliniMACS® Plus selection instrument where a magnetic field binds the bead-bound CD3+ T cells and removes them from the mixture. The depleted product was then formulated with 7.5% DMSO by adding CryoStor® CS10 Cryopreservation Freeze Medium.

### B. Characterization of Allogeneic CAR-Engineered T Cells

Engineered allogenic cells generated using methods described in part A above from 3 healthy human donors were assessed for expression of knock-in and knock-out efficiencies. HLA-E and CAR expression were assessed by flow cytometry. Knock-out of TRAC and B2M was assessed by droplet digital PCR (ddPCR).

Results are shown in Table E2 below. The engineered cells exhibited efficient knock-out of TRAC (as determined from low percent CD3+ surface expression) and B2M (as determined low percent positive HLA-ABC expression) in the primary T cells, as well as high level expression of the recombinant CAR and HLA-E fusion protein. The results also demonstrated a high level of viability, indicating that the methods of engineering and editing the cells did not impact cell survival. The results support the generation of T cells with high efficiency expression of an exemplary recombinant CAR and HLA-E when integrated at the endogenous TRAC and B2M loci, respectively.

TABLE E2
Knock-out and Knock-in Efficiency

| | | | CAR+ | | | | | | |
|---|---|---|---|---|---|---|---|---|---|
| | CAR | HLA- | HLA- | | HLA- | | | | |
| | KI | E(A)+ | E(A)+ | CD3+ | ABC+ | TRAC | B2M | Viability | CD4:CD8 |
| Donor | (%) | (%) | (%) | (%) | (%) | KO | KO | (%) | Ratio |
| 1 (Batch 1) | 90 | 80 | 74 | 0.46 | 1.17 | 99.2 | 98.9 | 74.6 | 0.42 |
| 2 (Batch 1) | 95 | 86 | 83 | 0.51 | 0.74 | 99.2 | 99.0 | 86.1 | 0.98 |
| 3 (Batch 1) | 85 | 69 | 62 | 0.68 | 1.61 | 99.0 | 98.8 | 79.3 | 0.59 |
| 1 (Batch 2) | 80 | 86 | 71 | 0.023 | 0.28 | 100 | 99.9 | 90.9 | 0.65 |
| 2 (Batch 2) | 84 | 87 | 75 | 0.019 | 0.24 | 100 | 99.9 | 94.7 | 1.46 |

Viability, CAR, HLA-E(A), CD3, and HLA-ABC expression levels were determined by flow cytometry where cryopreserved batches were thawed and stained to determine KO & KI efficiencies.
TRAC and B2M KO rates were determined by ddPCR.

### Example 3 Functionality of Recombinant CD19 CAR in Engineered Allogeneic T Cells

The engineered allogeneic T cells expressing CD19 CAR generated in Example 2 were tested for cytotoxic activity against target cells in a 3D tumor model using a spheroid killing assay.

Raji Burkitt's lymphoma cells (5,000 cells) were incubated for 24 h to allow formation of 3-D spheroid tumors. T cells were incubated with 3-D spheroid tumors at an effector to target (E:T) cell ratio of 1:2. Total integrated fluorescence intensity was measured in co-cultures (Panel A) by monitoring NucLight Red fluorescence for 6 days using IncuCyte® S3 Live-Cell Analysis System with IncuCyte® software (Essen Biosciences, Inc., Ann Arbor, MI). To determine target-cell lysis, total integrated intensity was plotted and the area under the curve (AUC) calculated using Prism software (GraphPad Software Inc., La Jolla, CA). Specific lysis was calculated using the formula % lysis=(100−[AUC for CAR T cells÷AUC for mock T cells×100]). Data are plotted for each donor. Proinflammatory cytokines were measured in supernatants collected 24 hours post co-culture with HD Allo CD19 CAR-T cells and 3-D spheroid tumors using an electrochemiluminescence cytokine immunoassay (Meso Scale Discovery, Rockville MD). IFN-γ, IL-2, and TNF-α cytokine concentrations were interpolated from a standard curve.

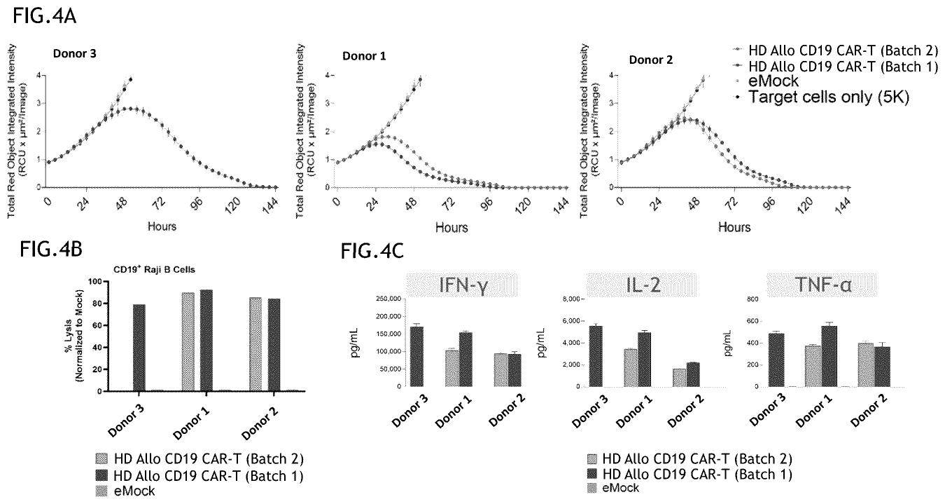

As shown in FIG. 4A, cytotoxic activity was observed for healthy donor allogeneic CD19 CAR T cells (HD Allo CD19 CAR-T cells) generated from the three individual donors, as compared to tumor cells alone. The percent lysis of Raji cells after 6 days of co-culture with Donor 1 (Batch 1), Donor 2 (Batch 1), and Donor 3 ranged from 79% to 92% (FIG. 4B). A similar range of cytotoxicity (85 to 90%) was observed with Donor 1 (Batch 2) and Donor 2 (Batch 2) (FIG. 4C). Similar levels of proinflammatory cytokines were measured following 24-hour co-culture with the HD Allo CD19 CAR-T cells from the three donors (FIG. 4C). A donor-dependent range in cytokine concentrations was observed.

### Example 4 In Vitro Cytolytic Activity of HD Allo CD19 CAR-T Cells Against B Cell Targets Isolated from SLE and Healthy Donor PBMCs

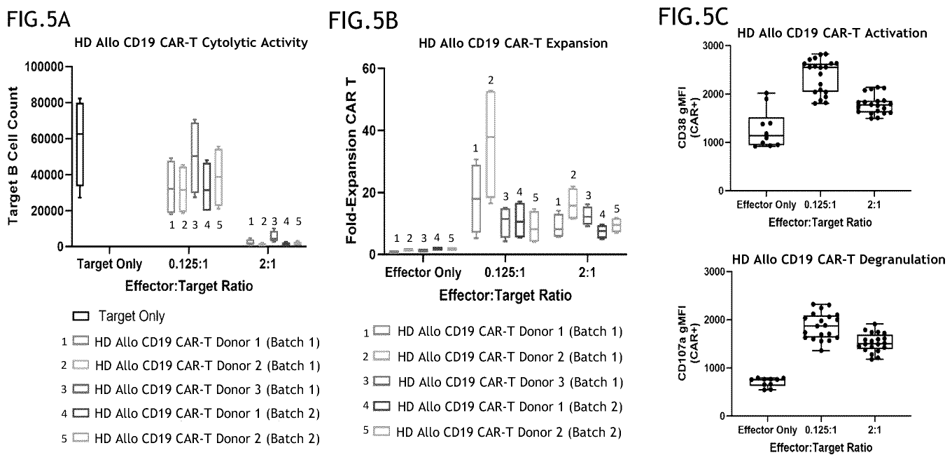

A flow-based in vitro cytotoxicity assay was performed with HD Allo CD19 CAR-T cells from Example 2 using B cell targets isolated from 3 different healthy donors and 1 SLE patient. B cells isolated from healthy and SLE donor material (3 HD; 1SLE) were fluorescently labeled with Carboxyfluorescein Diacetate Succinimidyl Ester (CFSE). Then the B cells were co-cultured with HD Allo CD19 CAR-T cells in vitro at an effector to target cell ratio of 0.125:1 (12.5K effectors and 100K targets) and 2:1 (200K effectors and 100K targets) for 72 h. Target B cell counts (Panel A) and fold-expansion of HD Allo CD19 CAR-T cells (Panel B) were measured by flow cytometry and quantified using CountBright Absolute Counting Beads. Target B cell counts were gated on CSFE+/Caspase3− cells and fold expansion of HD Allo CD19 CAR-T cells were gated on CFSE−/CAR+ cells. Fold expansion of HD Allo CD19 CAR-T cells was calculated by dividing the CAR-T cells counts at 72 h by CAR-T counts at 0 h. HD Allo CD19 CAR-T cell activation and degranulation (Panel C) were measured by quantification of CD38+CAR+gMFI and CD107a+CAR+gMFI, respectively. As shown in FIG. 5A, HD Allo CD19 CAR-T cells, in a dose-dependent manner, effectively depleted B cell targets from healthy and SLE donors. Also, B cell-dependent expansion of HD Allo CD19 CAR-T effector cells was also observed (FIG. 5B). In addition, increased HD Allo CD19 CAR-T cell activation and degranulation were observed by flow cytometry (FIG. 5C).

### Example 5 Assessment of TRAC Knock-Out in Engineered Allogeneic T Cells Against Graft-Vs-Host Disease (GvHD)

The main cellular mediators of both graft rejection (host vs graft, HvG) and GvH reactions are T cells that recognize non-self HLA molecules on allogeneic cells. To protect allogeneic T cells from rejection, HLA class I expression was eliminated by knocking out B2M, a universal component of all HLA class I molecules. To minimize GvH alloreactivity of the patient's tissues, the endogenous locus encoding the TRAC domain was disrupted using CRISPR-Cas9. Subsequently, the CD19 CAR was KI at the TRAC locus using HDR integration of the transgene sequence and delivered to T cells using an rAAV6 vector.

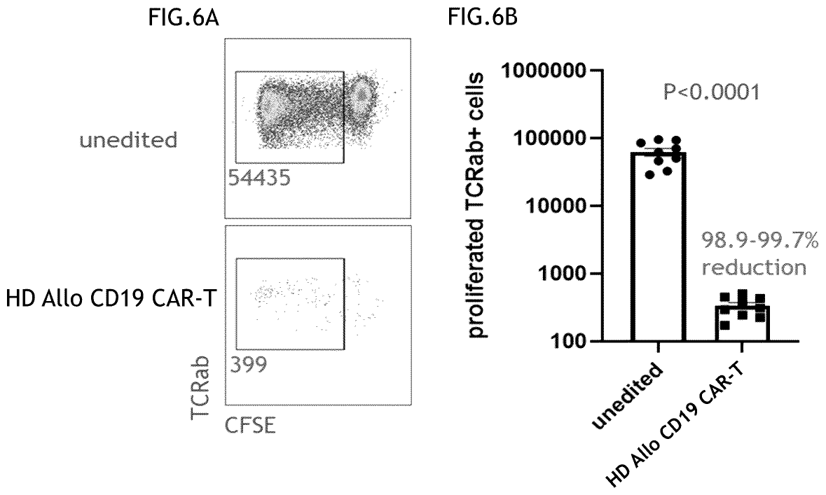

To assess the effect of TRAC KO on GvH alloreactivity, engineered allogeneic T cells generated as described in Example 2 were labeled with carboxyfluorescein succinimidyl ester (CSFE) as responder cells. CSFE-labeled, unedited mock T cells or HD Allo CD19 CAR-T cells were incubated with HLA-mismatched host dendritic cells in vitro for 6 days and the number of divided TCRab+ cells was measured by flow cytometry. Alloreactivity was determined by CSFE dilution (FIG. 6A).

Comparison of HD Allo CD19 CAR-T cells with paired unedited T cells from 3 healthy T cell donors in response to 3 allogeneic dendritic cell (DC) donors showed reduction of GvH alloreactivity of HD Allo CD19 CAR-T cells, ranging from 98.9% to 99.7% alloreactivity reduction (FIG. 6B). Attenuated T cell alloreactivity of the allogeneic T cells in response to HLA-mismatched dendritic cells indicates a reduced risk for development of graft-versus-host disease (GvHD).

### Example 6 Assessment of HLA-E Knock-In in Engineered Allogeneic T Cells Against NK-Mediated Host-vs-Graft Disease (GvHD)

Loss of class I MHC molecules on target cells has the ability to activate natural killer (NK) cells, leading to target cell killing and the potential for NK-mediated Host-vs-Graft (HvG) disease. Natural killer cells (NK) play an important role in nonadaptive immune responses by targeting cells that are infected, transformed, stressed, or recognized as “non-self”. They express a myriad of activating and inhibitory receptors that recognize the altered expression of proteins on target cells and regulate NK cytolytic function. The absence of class I MHC on target cells leads to the loss of NK inhibitory signals resulting in subsequent NK activation and target cell killing. The knock-out of B2M, and therefore the loss of HLA-I, on the engineered allogeneic T cells generated by methods described in Example 2 could result in NK-targeted killing of the allogeneic T cells.

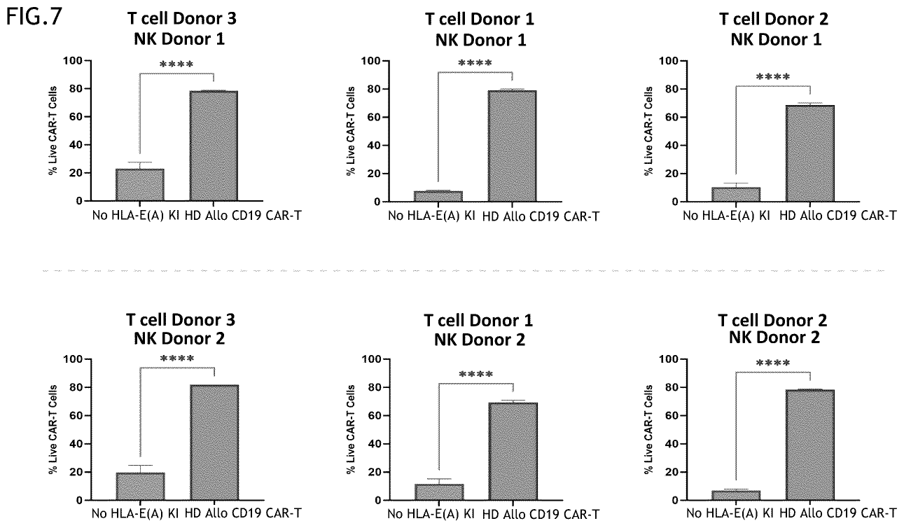

In this Example, experiments were performed to determine if knock-in of HLA-E at the B2M locus on the allogeneic T cells had the ability to counteract the NK cell activation against the engineered T cells. Engineered allogeneic T cells generated as described in Example 2 and control T cells (CAR+B2M KO/no HLA-E KI) were generated from 3 healthy donors and were plated at a 0.6:1 ratio with primary NK cells from 2 donors for 72 hours. After co-culture for 72 hours, the cells were analyzed by multicolor flow cytometry to identify the percentage of live CD5+/CD56−/CAR+ T cells. As shown in FIG. 7, allogeneic T cells with the knock-in of HLA-E had higher percentages of live cells after co-culture with NK cells, as compared to the control T cells (B2M KO/no HLA-E KI).

These results demonstrate the protective capacity of the HLA-E knock in against NK-mediated HvG disease.

### Example 7 In Vivo Activity of HD Allogeneic CD19 CAR T Cells

Engineered allogeneic T cells from two healthy donors generated as described in Example 2 were tested in a CD19-expressing Raji Burkitt's lymphoma xenograft tumor model in NOD.Cg-Prkdc^{scid}IL-^{2rgtm1Wj1}/SzJ immunodeficient mice (NSG). Two sub-curative doses of CAR T cells were selected to evaluated differential responses. NSG mice were injected intravenously via lateral tail vein injection on Study Day −7 with 5.0×10⁵ Raji Burkitt's lymphoma cells genetically engineered to express red-shifted Italian firefly luciferase (rFluc) to facilitate in vivo bioluminescent imaging. On Study Day −1, xenograft mice were strata rank-random allocated into groups (n=8 per group) based on tumor burden measured by whole-body bioluminescence imaging (BLI). The following day (Study Day 1), a single dose of 3.0×10⁶ or 1.0×10⁶ HD Allo CD19 CAR-T cells generated as described in Example 2 derived were administered by lateral tail vein infusion. Tumor growth was assessed in mice by imaging Raji firefly luciferase-positive bioluminescence and tumor burden was measured twice-weekly until study end.

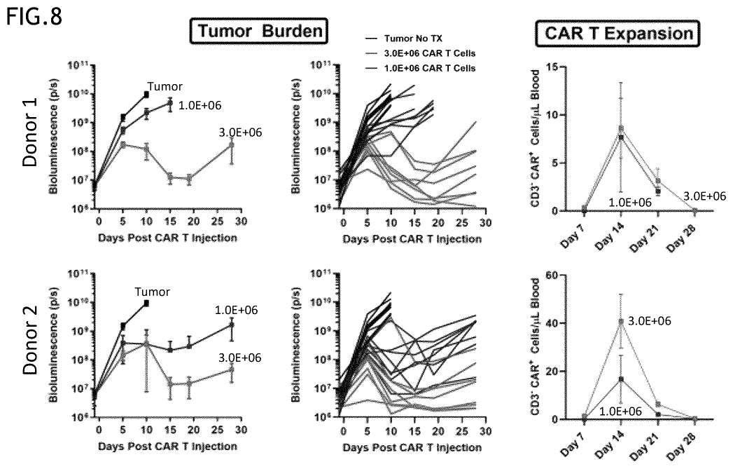

Changes in bioluminescence radiance (total flux [photons/second, p/s]; y-axis) are shown as individual tumor burden for all mice (n=8) in each dose group (FIG. 8). The number of live circulating CD45+/CD3−/CAR+ T cells in peripheral blood was measured using multicolor flow cytometry on study day 7, 14, 21, and 28. Data are displayed as group means±SEM. Statistical analysis was performed in GraphPad Prism 9.4, GraphPad Software, La Jolla, CA.

As shown in FIG. 8, the engineered allogeneic T cells from two different donors showed potent anti-tumor activity at 3.0×10⁶ CAR T dose, killing of CD19-expressing cells, and CAR T cell expansion kinetics.

### Example 8 Assessment of Residual Strep-Tactin Multimer Levels in Allogeneic CD19 CAR T Cell Product

Six lots of HD Allo CD19 CAR-T cell product were generated as described in Example 2. The Expamer activation reagent is made of three components: Strep-tactin backbone, CD3 Fabs, and CD28 Fabs. The amount of residual Strep-Tactin Multimer present in the final allogeneic HD Allo CD19 CAR-T cell product drug product was measured by an ELISA based assay using an anti-Streptavidin antibody that binds to Strep-Tactin Multimer (large streptavidin multimer). The amount of Strep-Tactin Multimer was assessed in cell lysate to quantitate total residual Strep-Tactin Multimer present in the final product. In one group of 3 lots, the range from low to high concentration of residual Strep-Tactin was 381-504 ng/mL. In another group of 3 lots, the range from low to high concentration of residual Strep-Tactin was 761-916 ng/mL.

### Example 9 Assessment of Residual Cas9 and AAV6 Levels in Allogeneic CD19 CAR T Cell Product

In the manufacture of HD Allo CD19 CAR-T cell product, cellular edits were introduced using the CRISPR-Cas9/Cas12a system and AAV6 vectors to generate 2 knockout events and 2 knock-in events as described in Example 2. After gene editing, the edited cells underwent 6 to 7 cumulative fold doublings and expansion during which perfusion and media exchange facilitated the removal of residuals. At harvest, cells were further washed prior to final formulation and cryopreservation.

Residual Cas9 and AAV6 were measured in the final drug product using target specific antibodies against the Cas9 and AAV6 capsid protein, respectively, using ELISA-based assays. In the Cas9 ELISA, anti-Cas9 antibodies were capable of binding both free Cas9 protein and complexed Cas9 (intact RNP). Similarly, the AAV6 ELISA was capable of detecting both full and empty AAV6 capsids. Input levels of Cas9 RNP (2.0 μM) were compared with Cas9 levels in cryopreserved product (2 ng/mL). Between input and final formulation, residual Cas9 underwent 2.21 logs of clearance. The concentration of residual Cas9 detected across six lots was below the limit of quantification of <2 ng/mL, which translated to <0.05 femtograms (fg)/cell. Clearance of AAV was calculated by comparing the input AAV capsid levels with capsid levels detected in lysates and supernatant prepared from final cryopreserved product. AAV6 underwent a 2.7 log clearance. The maximum concentration of residual AAV6 capsids detected across three cryopreserved product lots was 5.14×10¹¹ cp/mL.

### Example 10 Assessment of TRAC-B2M Translocations in Allogeneic CD19 CAR T Cell Product

Allogeneic CD19 CAR T cell product generated as described in Example 2 was assessed for on-target translocations between TRAC and B2M loci using a ddPCR assay. The ddPCR assay used target-specific primer-probe sets to detect each sub-type of TRAC-B2M translocation: balanced (1 and 2), acentric, and dicentric. Additionally, an external reference primer-probe set was utilized to calculate the percentage of translocation events out of total genome copies. The rates of the four sub-types were summed together to generate the total TRAC-B2M translocation frequency. In three representative lots of HD Allo CD19 CAR T cell product generated as described in Example 2, the total on-target TRAC-B2M translocations occurred on an average at 0.83%, with a range of <0.76%-0.89%.

### Example 11 Assessment of TRAC and B2M AAV6 Integration in Allogeneic CD19 CAR T Cell Product

HD Allo CD19 CAR T cell product generated as described in Example 2 was assessed for frequency of on-target rAAV6 integration at TRAC and B2M loci using a ddPCR assay. The ddPCR assay used target-specific primers and probes along with an AAV-specific inverted terminal repeat (ITR) primer. The ITR primer was designed to amplify across the junction of genomic DNA and integrated rAAV6 sequence. Since the ITR sequence was present in both rAAV6 vectors used in the manufacture of the allogeneic CD19 CAR T cell product, the assay was capable of detecting rAAV6 transgene at the on-target TRAC or B2M locus. Additionally, an external reference primer-probe set was utilized to calculate the percentage of rAAV6 integration out of total genome copies. On-target rAAV6 integration was quantified via ddPCR in 3 representative allogeneic CD19 CAR T cell product lots. On-target rAAV6 integration at the TRAC and B2M loci occurred on average at 1.72% and 0.93%, respectively.

### Example 12 Assessment of Off-Target Gene Editing in Allogeneic CD19 CAR T Cell Product

A genome-wide strategy was implemented to identify potential off-target sites for gene editing (indels) in HD Allo CD19 CAR T cell product generated as described in Example 2. The strategy involved a combination of three orthogonal genome-wide assays for each RNP. Potential candidate off-target sites, which included off-target sites identified by Digenome-Seq, GUIDE-Seq, in silico off-target sites with canonical protospacer-adjacent motif (PAM) with up to 3 mismatches, and off-target sites residing within coding regions, were evaluated. Each potential off-target site was analyzed in two lots from separate donors with paired unedited controls using rhAmpSeq. Editing frequency was calculated as the percentage of total reads containing an indel within a 10 bp window on either side of the predicted cut site. A site was considered off-target if the indel frequency was >0.2% and had a false discovery rate (FDR)<0.05 in the edited versus the unedited control sample. No off-target sites were confirmed in either lot of HD Allo CD19 CAR T cell product.

### Example 13 Assessment of Off-Target AAV Integration in Allogeneic CD19 CAR T Cell Product

Genome-wide AAV integration was qualitatively assessed using a modified version of the ITR-Seq method, an NGS-based method that identifies the genomic location of insertions of specific AAV vector inverted terminal repeats (ITRs). Genomic DNA from paired edited and unedited donors was enzymatically fragmented. Custom Y-forked adapters were ligated to both ends of the DNA. Next, end-repaired Y-adapter-ligated DNA fragments were subjected to two rounds of PCR. The first PCR used ITR- and adapter-specific primers for targeted amplification of the genomic DNA junction and inserted vector ITR sequence. The second round of PCR incorporated adapters to each amplicon for universal amplification. NGS libraries were size selected, quantified by qPCR, and paired-end sequenced. No off-target AAV integration was observed at genomic sites homologous to the gRNA targeting sequences.

### Example 14 Phase 1 Study to Determine the Safety, Tolerability, and Preliminary Efficacy of Healthy Donor Allogeneic CD19 CAR T Cell Product in Subjects with Severe, Refractory Autoimmune Diseases

An open-label, Phase 1 study to determine the optimal dose, safety, tolerability, and preliminary efficacy of HD Allo CD19 CAR T cell product is evaluated in subjects with severe, refractory autoimmune diseases. A basket study design is used to test the safety and preliminary efficacy of CD19-targeted allogeneic CAR T cells in treating severe, life-threatening cases of systemic lupus erythematosus (SLE), idiopathic inflammatory myopathy (IIM), systemic sclerosis (SSc), or rheumatoid arthritis (RA).

Patients diagnosed with SLE and SLE disease activity are eligible to be included in the study. SLE is diagnosed as:
Fulfilling the 2019 ACR/EULAR classification criteria of SLE, described in Aringer M, et al. 2019 European League Against Rheumatism/American College of Rheumatology Classification Criteria for Systemic Lupus Erythematosus. Arthritis Rheumatol 2019; 71:1400-12, which is incorporated by reference in its entirety.
Presence of anti-dsDNA, anti-histone, anti-chromatin, or anti-Sm antibodies at screening.

SLE disease activity is diagnosed as:
Active disease at screening, defined as ≥1 major organ system with a British Isles Lupus Assessment (BILAG A) or ≥2 BILAG B scores. The BILAG index is a computerized index for measuring clinical disease activity of lupus over the past 4 weeks. It includes 97 features, divided into 9 organ/body systems. All organ systems carry equal weight and are answered as 0=not present, 1=improving, 2=same, 3=worse, and 4=new. Then, a computer program facilitates scoring from numerical to alphabetical score for each system (Grade A-E) where Grade A represents very active disease and Grade E indicates no current disease activity. BILAG is performed at the screening visit.
Inadequate response to glucocorticoids and at least 2 of the following treatments, used for at least 3 months each: cyclophosphamide, mycophenolic acid or its derivatives, belimumab, azathioprine, anifrolumab, methotrexate, rituximab, binutuzumab, cyclosporin, tacrolimus, or voclosporin.

Patients diagnosed with IIM and IIM disease activity are eligible to be included in the study. IIM is diagnosed as:
Fulfilling the 2017 ACR/EULAR classification criteria for probable or definite IIM, described in Lundberg I E, et al., 2017 European League Against Rheumatism/American College of Rheumatology classification criteria for adult and juvenile idiopathic inflammatory myopathies and their major subgroups. Ann Rheum Dis. 2017 December; 76(12):1955-1964, which is incorporated by reference in its entirety.
Subject diagnosed with one of the following IIM subgroups:
(i) dermatomyositis (DM): history of heliotrope rash or Gottron's papules or sign;
(ii) immune-mediated necrotizing myopathy (IMNM): muscle biopsy consistent with necrotizing myopathy or prior history of either anti-HMGCR or anti-SRP autoantibody;
(iii) antisynthetase syndrome (AsyS): defined as the history of anti-synthetase autoantibody and at least 12 of the following: progressive ILD, inflammatory myositis, or inflammatory polyarthritis;
(iv) polymyositis (PM): defined as patients with symmetric proximal more than distal muscle weakness or upper and lower extremity, CK elevation, and other features like myopathic EMG findings, evidence of myositis on muscle MRI, and an appropriate muscle biopsy, who do not have characteristics of other form of IIMs.
Presence of at least 1 myositis specific antibody (MSA), myositis associated antibody (MAA), or antinuclear antibody (ANA) at screening or prior to screening.

IIM disease activity is diagnosed as (1) and (2) below:
(1) Severe muscle AND/OR skin involvement defined as: Manual Muscle Testing-8 (MMT-8)<130 or <136 (severe) or CDASI of >14 or >19 for skin AND MMT-8<142; and either (A) evidence of active disease as documented by any one of the following:
(i) active dermatomyositis-associated rashes (Gottron's papules or heliotrope rash, and other DM rashes) and CDASI-A≥6 OR
(ii) a recent muscle biopsy (within 3 months prior to signing informed consent), magnetic resonance imaging, or electromyogram demonstrating active disease (documentation must be obtained and retained by the site) OR
(iii) an elevated CK>4 times the ULN at screening with no alternate explanation or cause; or (B) participants diagnosed with IIM and progressive interstitial lung disease (ILD).
(2) Inadequate response to glucocorticoids and at least 2 of the following treatments used for at least 3 months: azathioprine, methotrexate, cyclosporin A, tacrolimus, mycophenolate mofetil (MMF), cyclophosphamide, intravenous immunoglobulin (IVIG), JAK inhibitors, and rituximab, wherein at least one of the treatments is MMF, tacrolimus, cyclosporin A, cyclophosphamide, or rituximab.

Patients diagnosed with SSc and SSc disease activity are eligible to be included in the study. SSc is diagnosed as:
(1) Fulfilling the 2013 ACR/EULAR classification criteria for SSc, described in van den Hoogen F., et al. 2013 classification criteria for systemic sclerosis: an American College of Rheumatology/European League Against Rheumatism Collaborative Initiative. Ann Rheum Dis 2013; 72:1747-55, which is incorporated by reference in its entirety.
(2) Presence of one or more of the following antibodies at screening: anti-nuclear (ANA), anticentromere, anti-topoisomerase (anti-Scl-70), or anti-RNA polymerase III

SSc disease is diagnosed as either (1) or (2), and (3) below:
(1) Disease duration≤7 years (from onset of first non-Raynaud manifestation), modified Rodnan Skin Score (mRSS)≥15 at screening AND mRSS increase≥3 units or involvement of 1 new body area, mRSS increase≥2 units in 1 body area over 6 months, mRSS>35 at screening, and C-reactive protein (CRP)≥10.0 mg/L, erythrocyte sedimentation rate≥28 mm/h, or HAQ-DI>1.0.
(2) Participants diagnosed with limited or diffuse cutaneous SSc AND progressive ILD with disease duration≤7 years (from onset of first nonRaynaud manifestation).
(3) Inadequate response to at least 1 of the following treatments used for at least 3 months: methotrexate, nintedanib, mycophenolate, cyclophosphamide, rituximab, or tocilizumab.

Patients diagnosed with RA and RA disease activity are eligible to be included in the study. RA is diagnosed as follows:
Fulfilling 2010 ACR/EULAR criteria for RA.
ACPA and/or RF positivity at screening or prior to screening.

RA disease is diagnosed as (1), either (2) or (3), and (4) below:
(1) Minimum of 3 swollen (SJC) and 3 tender joints (TJC) on a 66/68 joint count at the screening and baseline visit (SJC/TJC) AND
(2) 28-joint disease activity score-C reactive protein (DAS-CRP)
   Moderate DAS-CRP (>3.2), with extraarticular manifestations, including but not limited to RA-ILD OR
   High DAS-CRP (>5.1) with or without extraarticular manifestations; or
(3) participants diagnosed with progressive ILD; and
(4) Inadequate response to at least 2 biologic disease-modifying anti-rheumatic drug(s) (bDMARDs) or targeted synthetic disease-modifying anti-rheumatic drug(s) (tsDMARDs) with different mechanisms of action after failing conventional synthetic DMARD. An inadequate disease response requires a minimum of 3 months of a given therapy. Biologic DMARDs and targeted synthetic DMARDs as defined by EULAR.

The study consists of 2 parts: a dose escalation (Part A) and a dose-expansion part (Part B). Part A evaluates the safety and tolerability of increasing doses of CD19-targeted allogeneic CAR T cells in a single administration to establish the dose for expansion. Part B further evaluates the safety, PK/pharmacodynamics, and preliminary efficacy of CD19-targeted allogeneic CAR T cells at one or more recommended Phase 2 doses (RP2Ds). One or more doses is chosen from Part A as preliminary RP2D(s) for further evaluation in Part B in each indication. The study has 3 periods: Screening (Pre-treatment), Treatment, and Post-treatment Follow-up.

In the treatment period, subjects with severe, refractory systemic lupus erythematosus (SLE), idiopathic inflammatory myopathy (IIM), systemic sclerosis (SSc), and rheumatoid arthritis (RA) are treated with fludarabine IV (30 mg/m²/day) and cyclophosphamide IV (300 mg/m²/day) for 3 days prior to infusion of CD19-targeted allogeneic CAR T cells (Days −5, −4, and −3). In the dose escalation part of the study, 4 escalating dose levels (100×10⁶, 200×10⁶, 300×10⁶, and 450×10⁶ CD19-targeted allogeneic CAR T cells) are administered by IV on Day 1 to different cohorts. A dose level of 50×10⁶ CD19-targeted allogeneic CAR T cells may also be administered.

Efficacy of CD19-targeted allogeneic CAR T cells is evaluated in subjects having SLE, IIM, SSc, or RA with ILD with no worsening of pulmonary function, including FVC decline>10% from baseline to Week 24.

Efficacy of CD19-targeted allogeneic CAR T cells is evaluated in subjects with severe, refractory SLE by the following:
Proportion of subjects achieving DORIS remission at Week 24.
Proportion of subjects achieving LLDAS at Week 24.
Change in proteinuria (UPCR) from baseline to Week 24.
Change in HAQ-DI at Week 24 from baseline.

### DORIS

The DORIS is the product of an effort to define remission in SLE. It was achieved by the DORIS Task Force which first convened in 2015. It was based on systematic literature reviews and other analyses including analysis of observational cohorts, registries, and clinical trial data sets. The DORIS criteria is based on the Systemic Lupus Erythematosus Disease Activity Index-2000 (SLEDAI-2K), the Physician's Global Assessment of Disease Activity (PhGA) (0-3), and achieving stable lupus-specific therapies irrespective of persistent serology. The 2021 DORIS definition of remission in SLE is a) Clinical SLEDAI=0; b) PhGA<0.5 (from the 0-3 visual analog scale (VAS) scale) irrespective of serologies; and c) the subject may be on antimalarials, low-dose glucocorticoids (prednisolone≤5 mg/day), and/or stable immunosuppressives including biologics. The DORIS has been further subdivided into the “DORIS clinical remission on-treatment” and the “DORIS complete remission.” The former refers to the initial definition while the latter allows for the following modifications of the definition: a) no serological activity is allowed; b) no glucocorticoids allowed; and c) maintenance antimalarials are allowed, but no immunosuppressives and/or biologics are allowed. In this study, the DORIS remission is calculated using both definitions.

### Lupus Low Disease Activity State (LLDAS)

The development of the LLDAS was driven by the recognition that a treatment-related goal similar to that used in for RA was desirable for use with subjects with SLE. The LLDAS definition was achieved with a consensus generated using Delphi and nominal group techniques.

LLDAS is:
SLEDAI-2K≤4, with no activity in major organ systems (renal, CNS, cardiopulmonary, vasculitis, fever) and no hemolytic anemia or gastrointestinal activity
No new lupus disease activity compared with the previous assessment
A Safety of Estrogens in Lupus Erythematosus National Assessment (SELENA)-SLEDAI physician global assessment (scale 0 to 3)≤1
A current prednisolone (or equivalent) dose≤7.5 mg daily
Well-tolerated standard maintenance doses of immunosuppressive drugs and approved biological agents.

### Systemic Lupus Erythematosus Disease Activity Index-2000 (SLEDAI2K)

The SLEDAI is a clinical index for the assessment of lupus disease activity in the past 10 days. It consists of 24 weighted clinical and laboratory variables of 9 organ systems. The scores of the descriptors range from 1 to 8, and the total possible score for all 24 descriptors is 105. In this study, the SLEDAI-2K version is used.

### Physician's Global Assessment of Disease Activity (PhGA), 0-3 VAS

The physician is asked to place a vertical line on a 0-3 cm VAS on which the left-hand boundary represents “no disease activity,” and the right-hand boundary represents “maximum disease activity.” The distance from the mark to the left-hand boundary is recorded.

### Health Assessment Questionnaire-Disability Index (HAQ-DI)

The HAQ-DI assesses a subject's level of functional ability and includes questions of fine movements of the upper extremity, locomotor activities of the lower extremity, and activities that involve both upper and lower extremities. There are 20 questions in 8 categories of functioning, which represent a comprehensive set of functional activities—dressing, rising, eating, walking, hygiene, reach, grip, and usual activities. The stem of each item asks for the past week “Are you able to . . . ” perform a particular task. The subject's responses are made on a scale from 0 (no disability) to 3 (completely disabled). Each category contains at least 2 specific component questions. The HAQ-DI has been validated for use in many diseases including in patients with autoimmune diseases. The same instrument is used for assessment in subjects with SLE, IIM, SSc or RA.

Efficacy of CD19-targeted allogeneic CAR T cells is evaluated in subjects with systemic sclerosis by the following:
Proportion of subjects achieving a minimal clinically important differences (MCID) of 24% change from baseline of the mRSS at Week 24.
Change in HAQ-DI at Week 24 from baseline.
Improvement from baseline of the Revised CRISS at Week 24.

Efficacy of SSc is assessed by the extent and severity of skin involvement using a SSc-specific instrument, the mRSS, along with other measures. Due to the frequency and clinical impact of lung involvement, assessments of lung function are included. Most of these comprise the measures for calculating the Revised Composite Response Index in Systemic Sclerosis (CRISS) composite measure.

Modified Rodnan Skin Score (mRSS)

The mRSS is a measure of skin thickness used in subjects with SSc using a 0 to 3 scale in 17 body areas (scale 0 to 51). The body areas have been chosen since they were easy to perform, did not require equipment (e.g., ultrasound), cover the affected body areas, and have acceptable inter-observer variability. The scale ranges from normal skin to severe skin thickness with inability to make skin folds between 2 examining fingers. The minimal clinically important differences (MCID) is an improvement of 24%.

### Revised CRISS

The revised CRISS is a composite endpoint to assess the likelihood of improvement in diffuse systemic sclerosis. It addresses the limitation of the mRSS and other outcomes measured used in SSc trials. This was updated when ceiling and floor effects that could impact responsiveness to change were identified. The revised CRISS is a global measure based on a probability score of 0.0 (no improvement) to 1.0 (marked improvement). It includes 2 steps. In step 1, worsening or incident cases of cardio-pulmonary-renal involvement are assessed and given a score of 0.0. For those who are not considered to have worsened in Step 1, a weighted probability score is calculated in Step 2. This score incorporates changes from the 5 core measures: mRSS, forced vital capacity (FVC) % predicted (from pulmonary function test (PFTs)), HAQ-DI, subject global assessment, and physician global assessment. Improvement as a percentage of baseline values is determined. A cut-off of ≥25% for mRSS, Physician Global Assessments (PhGA) (0 to 10), PtGA (1 to 10), and HAQ-DI and ≥5% for FVC, change from baselines, in Step 2, is used. Subjects are considered to have met the definition of improvement if there is improvement from baseline for at least 2 of 5 of the core set measures and not more than 1 core measure worsening by the same criteria.

Physician Global Assessments (PhGA), 0 to 10 VAS measures the global evaluation by the treating physician of the overall disease activity of the subject at the time of the assessment using a 10-cm VAS that can be performed by the Investigator or sub-investigator. This has been validated for use in subjects with SSc. Responders globally assess overall disease activity or the way that SSc has affected the subject. A change of 1 unit is considered as the MCID on the 0 to 10 scale. The PhGA (0 to 10) used for subjects with SSc is similar to that used in subjects with IIM.

Patient Global Assessment of Disease Activity (PtGA; 0 to 10), measures the subjects' global evaluation of their overall disease activity at the time of the assessment using a continuous VAS that is 10 cm (or 0 to 10) long. The PtGA (0 or 10) is used for subjects with IIM, SSc, or RA.

Efficacy of CD19-targeted allogeneic CAR T cells is evaluated in subjects with severe, refractory IIM by the following:
Proportion of subjects achieving MRC TIS Moderate or Major Response at Week 24.
Change in IMACS outcome measure set for disease activity at Week 24 from baseline.
Change in CDASI (DM subjects only) at Week 24 from baseline.

Efficacy outcomes for subjects with IIM is assessed by the International Myositis Outcome Assessment Collaborative Study Group (IMACS) outcome measure set: MMT-8, PhGA (0 to 10), Patient Global Assessment of Disease Activity (PtGA) (0 to 10), HAQ-DI, Myositis Disease Activity Assessment Tool (MDAAT) Extramuscular Global Assessment, and creatine kinase (CK). Skin disease in subjects with dermatomyositis (DM) is assessed using the CDASI instrument. Lung function is assessed with pulmonary function test (PFT) and high-resolution computed tomography (HRCT). Autoantibodies seen in subjects with IIM is assessed.

### Myositis Response Criteria (MRC) Total Improvement Score (TIS)

The MRC algorithm generates a continuous total improvement score (range 0 to 100) based on the sum of the absolute percent change in the 6 core domains (weighted) used in the IMACS definition of improvement (DOI): MMT-8, PhGA (0 to 10), PtGA (0 to 10), HAQ-DI, MDAAT Extramuscular Global Assessment and creatine kinase (CK). It also defines cut points for minimal (≥20 point increase), moderate (≥40 points), and major (≥60 points) improvement in disease.

### Cutaneous Dermatomyositis Disease Area and Severity Index (CDASI)

This tool assesses improvement in skin disease activity in subjects with dermatomyositis (DM). Four domains are evaluated, including: 1) disease activity and damage in anatomic locations, 2) presence of Gottron's papules on the hands, 3) periungual changes, and 4) alopecia.

Efficacy of CD19-targeted allogeneic CAR T cells is evaluated in subjects with severe, refractory RA by the following:
Proportion of subjects achieving low DAS28-CRP at Week 24 from baseline.
Change in HAQ-DI at Week 24 from baseline.

### Disease Activity Score in Rheumatoid Arthritis

The Disease Activity Score (DAS) can be used to assess subjects RA disease activity, determine whether it is under control and if any treatment adjustments are required. It can also assist in establishing a target score to aim for, help inform treatment decisions, and optimize disease management. DAS28-CRP are composite outcome assessments that measure:
How many joints in the hands (including metacarpophalangeal and proximal interphalangeal joints but excluding distal interphalangeal joints), wrists, elbows, shoulders, and knees are swollen and/or tender over a total of 28.
CRP, or erythrocyte sedimentation rate (ESR), in the blood to measure the degree of inflammation.
PtGA of disease activity.
The results are combined to produce the DAS28-CRP (or DAS28-ESR) score, which correlates with the extent of disease activity:
   <2.6: Disease remission
   2.6 to 3.2: Low disease activity
   3.2 to 5.1: Moderate disease activity
   >5.1: High disease activity

Myositis Disease Activity Assessment Tool (MDAAT) Extramuscular Global Assessment is a combined tool that captures the physician's assessment of disease activity of various organ systems using (1) a 0 to 4 scale, and (2) a VAS. The Extramuscular Global Assessment VAS evaluates overall disease activity in all the extramuscular organ systems and excludes muscle disease activity. The overall (global) assessment of the ongoing disease activity over the past 4 weeks is done on the 0 cm to 10 cm VAS scale by drawing a vertical mark on the 10-cm line.

SEQUENCE TABLE

| SEQ | | |
|---|---|---|
| ID | | |
| NO: | Sequence | Description |
| 1 | EVKLQESGPGLVAPSQSLSVTCTVSGVSLPDYGVSWIRQPPRKGLEWLGVIWGSETTYYNSALKSRLTIIK | CD19 VH |
| | DNSKSQVFLKMNSLQTDDTAIYYCAKHYYYGGSYAMDYWGQGTSVTVSS | |
| 2 | DIQMTQTTSSLSASLGDRVTISCRASQDISKYLNWYQQKPDGTVKLLIYHTSRLHSGVPSRFSGSGSGTDY | CD19 VL |
| | SLTISNLEQEDIATYFCQQGNTLPYTFGGGTKLEIT | |
| 3 | DYGVS | CD19 CDR-H1 |
| 4 | VIWGSETTYYNSALKS | CD19 CDR-H2 |
| 5 | YAMDYWG | CD19 CDR-H3 |
| 6 | HYYYGGSYAMDY | CD19 CDR-H3 |
| 7 | RASQDISKYLN | CD19 CDR-L1 |
| 8 | SRLHSGV | CD19 CDR-L2 |
| 9 | HTSRLHS | CD19 CDR-L2 |
| 10 | GNTLPYTFG | CD19 CDR-L3 |
| 11 | QQGNTLPYT | CD19 CDR-L3 |
| 12 | ESKYGPPCPPCPM | Short Spacer |
| | | (IgG4 hinge) |
| | | (aa) |
| 13 | ESKYGPPCPPCPAPPVAGPSVFLFPPKPKDTLMISRTPEVTCVVVDVSQEDPEVQFNWYVDGVEVHNAKTK | Long spacer |
| | PREEQFQSTYRVVSVLTVLHQDWLNGKEYKCKVSNKGLPSSIEKTISKAKGQPREPQVYTLPPSQEEMTKN | (IgG4/IgG2 |
| | QVSLTCLVKGFYPSDIAVEWESNGQPENNYKTTPPVLDSDGSFFLYSRLTVDKSRWQEGNVFSCSVMHEAL | hinge- |
| | HNHYTQKSLSLSLGK | IgG2/IgG4 |
| | | CH2-IgG4 |
| | | CH3 spacer |
| 14 | gaatctaagtacggaccgccctgccctccctgccctgctcctcctgtggctggaccaagcgtgttcctgtt | IgG4/IgG2 |
| | tccacctaagcctaaagataccctgatgatttcccgcacacctgaagtgacttgcgtggtcgtggacgtga | hinge- |
| | gccaggaggatccagaagtgcagttcaactggtacgtggacggcgtggaagtccacaatgctaagactaaa | IgG2/IgG4 |
| | ccccgagaggaacagtttcagtcaacttaccgggtcgtgagcgtgctgaccgtcctgcatcaggattggct | CH2-IgG4 |
| | gaacgggaaggagtataagtgcaaagtgtctaataagggactgcctagctccatcgagaaaacaattagta | CH3 spacer |
| | aggcaaaagggcagcctcgagaaccacaggtgtataccctgccccctagccaggaggaaatgaccaagaac | (nt) |
| | caggtgtccctgacatgtctggtcaaaggcttctatccaagtacatcgccgtggagtgggaatcaaatggg | |
| | cagcccgagaacaattacaagaccacaccacccgtgctggactctgatggaagtttctttctgtattccag | |
| | gctgaccgtggataaatctcgctggcaggagggcaacgtgttctcttgcagtgtcatgcacgaagccctgc | |
| | acaatcattatacacagaagtcactgagcctgtccctgggcaaa | |
| 15 | FWVLVVVGGVLACYSLLVTVAFIIFWV | CD28 |
| | | transmembrane |
| | | domain (aa) |
| 16 | KRGRKKLLYIFKQPFMRPVQTTQEEDGCSCRFPEEEEGGCEL | 4-1BB-derived |
| | | intracellular |
| | | costimulatory |
| | | domain(aa) |
| 17 | RVKFSRSADAPAYQQGQNQLYNELNLGRREEYDVLDKRRGRDPEMGGKPRRKNPQEGLYNELQKDKMAEAY | CD3-zeta |
| | SEIGMKGERRRGKGHDGLYQGLSTATKDTYDALHMQALPPR | derived |
| | | intracellular |
| | | signaling |
| | | domain (aa) |
| 18 | GGGGSGGGGSGGGGS | Linker |
| 19 | GSTSGSGKPGSGEGSTKG | Linker |
| 20 | GGGGS | Linker |
| 21 | GGGGSGGGGS | Linker |
| 22 | GGGGSGGGGSGGGGSGGGGS | Linker |
| 23 | EVQLVESGGGLVQPGRSLRLSCAASGFTFDDYAMHWVRQAPGKGLEWVSGISWNSGRIGYADSVKGRFTIS | CD19 clone 5 |
| | RDNAKNSLFLQMNSLRAEDTAVYYCARDQGYHYYDSAEHAFDIWGQGTMVTVSS | V_{H}; |
| | | CD19 clone 17 |
| | | V_{H} |
| 24 | EVQLVESGGGLVQPGRSLRLSCAASGFTFDDYAMHWVRQAPGKGLEWVSGISWNSGRIGYADSVKGRFTIS | CD19 clone 18 |
| | RDNAKNSLFLQMNSLRAEDTAVYYCARDQGYHYYDSAEHAFDIWGQGTVVTVSS | V_{H}; |
| | | CD19 clone |
| | | 18B V_{H}; |
| | | CD19 clone 76 |
| | | V_{H} |
| 25 | SYELTQDPAVSVALGQTVRITCQGDSLRSYYASWYQQKPGQAPVLVIYDKNNRPSGIPDRFSGSSSGNTAS | CD19 clone 5 |
| | LTITGAQAEDEADYYCNSRDSSGNNWVFGGGTKLTVL | V_{L} |
| 26 | QSALTQPASVSGSPGQSITIFCTGTSSDVGGYNYVSWYQQLPGTAPKLLIYSNNQRPSGVPDRFSGSKSGT | CD19 clone 17 |
| | SASLAISGLRSEDEADYYCAAWDDSLSVVFGGGTKLTVL | V_{L} |
| 27 | QSALTQPRSVSGFPGQSVTISCTGTTSDDVSWYQQHPGKAPQLMLYDVSKRPSGVPHRFSGSRSGRAASLI | CD19 clone 18 |
| | ISGLQTEDEADYFCCSYAGRYNSVLFGGGTKLTVL | V_{L} |
| 28 | QSALTQPRSVSGFPGQSVTISCTGTTSDDVSWYQQHPGKAPQLMLYDVSKRPSGVPHRFSGSRSGRAASLI | CD19 clone |
| | ISGLQTEDEADYFCSSYAGRYNSVLFGGGTKLTVL | 18B V_{L} |
| 29 | QSVLTQPPSVSAAPGQEVTISCSGSSSNIGNNYVSWYQQLPGTAPKLLIYDNDKRPSGIPDRFSGSKSGTS | CD19 clone 76 |
| | ATLGITGLQTGDEADYYCGTWDGNLSAVFGGGTKVTVL | V_{L} |
| 30 | EVQLVESGGGLVQPGRSLRLSCAASGFTFDDYAMHWVRQAPGKGLEWVSGISWNSGRIGYADSVKGRFTIS | CD19 clone 5 |
| | RDNAKNSLFLQMNSLRAEDTAVYYCARDQGYHYYDSAEHAFDIWGQGTMVTVSSGGGGSGGGGSGGGGSSY | scFv |
| | ELTQDPAVSVALGQTVRITCQGDSLRSYYASWYQQKPGQAPVLVIYDKNNRPSGIPDRFSGSSSGNTASLT | |
| | ITGAQAEDEADYYCNSRDSSGNNWVFGGGTKLTVL | |
| 31 | EVQLVESGGGLVQPGRSLRLSCAASGFTFDDYAMHWVRQAPGKGLEWVSGISWNSGRIGYADSVKGRFTIS | CD19 clone 17 |
| | RDNAKNSLFLQMNSLRAEDTAVYYCARDQGYHYYDSAEHAFDIWGQGTMVTVSSGGGGSGGGGSGGGGSQS | scFv |
| | ALTQPASVSGSPGQSITIFCTGTSSDVGGYNYVSWYQQLPGTAPKLLIYSNNQRPSGVPDRFSGSKSGTSA | |
| | SLAISGLRSEDEADYYCAAWDDSLSVVFGGGTKLTVL | |
| 32 | EVQLVESGGGLVQPGRSLRLSCAASGFTFDDYAMHWVRQAPGKGLEWVSGISWNSGRIGYADSVKGRFTIS | CD19 clone 18 |
| | RDNAKNSLFLQMNSLRAEDTAVYYCARDQGYHYYDSAEHAFDIWGQGTVVTVSSGGGGSGGGGSGGGGSQS | scFv |
| | ALTQPRSVSGFPGQSVTISCTGTTSDDVSWYQQHPGKAPQLMLYDVSKRPSGVPHRFSGSRSGRAASLIIS | |
| | GLQTEDEADYFCCSYAGRYNSVLFGGGTKLTVL | |
| 33 | EVQLVESGGGLVQPGRSLRLSCAASGFTFDDYAMHWVRQAPGKGLEWVSGISWNSGRIGYADSVKGRFTIS | CD19 clone |
| | RDNAKNSLFLQMNSLRAEDTAVYYCARDQGYHYYDSAEHAFDIWGQGTVVTVSSGGGGSGGGGSGGGGSQS | 18B scFv |
| | ALTQPRSVSGFPGQSVTISCTGTTSDDVSWYQQHPGKAPQLMLYDVSKRPSGVPHRFSGSRSGRAASLIIS | |
| | GLQTEDEADYFCSSYAGRYNSVLFGGGTKLTVL | |
| 34 | EVQLVESGGGLVQPGRSLRLSCAASGFTFDDYAMHWVRQAPGKGLEWVSGISWNSGRIGYADSVKGRFTIS | CD19 clone 76 |
| | RDNAKNSLFLQMNSLRAEDTAVYYCARDQGYHYYDSAEHAFDIWGQGTVVTVSSGGGGSGGGGSGGGGSQS | scFv |
| | VLTQPPSVSAAPGQEVTISCSGSSSNIGNNYVSWYQQLPGTAPKLLIYDNDKRPSGIPDRFSGSKSGTSAT | |
| | LGITGLQTGDEADYYCGTWDGNLSAVFGGGTKVTVL | |
| 35 | DYAMH | CD19 clone 5, |
| | | 17, 18, 18B, 76 |
| | | VH CDR1 |
| 36 | GISWNSGRIGYADSVKG | CD19 clone 5, |
| | | 17, 18, 18B, 76 |
| | | VH CDR2 |
| 37 | DQGYHYYDSAEHAFDI | CD19 clone 5, |
| | | 17, 18, 18B, 76 |
| | | VH CDR3 |
| 38 | QGDSLRSYYAS | CD19 clone 5 |
| | | VL CDR1 |
| 39 | DKNNRPS | CD19 clone 5 |
| | | VL CDR2 |
| 40 | NSRDSSGNNWV | CD19 clone 5 |
| | | VL CDR3 |
| 41 | TGTSSDVGGYNYVS | CD19 clone 17 |
| | | VL CDR1 |
| 42 | SNNQRPS | CD19 clone 17 |
| | | VL CDR2 |
| 43 | AAWDDSLSVV | CD19 clone 17 |
| | | VL CDR3 |
| 44 | TGTTSDDVS | CD19 clone 18 |
| | | VL CDR1; |
| | | CD19 clone |
| | | 18B VL CDR1 |
| 45 | DVSKRPS | CD19 clone 18 |
| | | VL CDR2; |
| | | CD19 clone |
| | | 18B VL CDR2 |
| 46 | CSYAGRYNSVL | CD19 clone 18 |
| | | VL CDR3 |
| 47 | SSYAGRYNSVL | CD19 clone |
| | | 18B VL CDR3 |
| 48 | SGSSSNIGNNYVS | CD19 clone 76 |
| | | VL CDR1 |
| 49 | DNDKRPS | CD19 clone 76 |
| | | VL CDR2 |
| 50 | GTWDGNLSAV | CD19 clone 76 |
| | | VL CDR3 |
| 51 | X1PPX2P, X1 is glycine, cysteine or arginine and X2 is cysteine or | Hinge |
| | threonine | consensus |
| 52 | ASTKGPSVFPLAPCSRSTSESTAALGCLVKDYFPEPVTVSWNSGALTSGVHTFPAVLQSSGLYSLSSVVTV | Human IgG2 |
| | PSSNFGTQTYTCNVDHKPSNTKVDKTVERKCCVECPPCPAPPVAGPSVFLFPPKPKDTLMISRTPEVTCVV | Fc (Uniprot |
| | VDVSHEDPEVQFNWYVDGVEVHNAKTKPREEQFNSTFRVVSVLTVVHQDWLNGKEYKCKVSNKGLPAPIEK | P01859) |
| | TISKTKGQPREPQVYTLPPSREEMTKNQVSLTCLVKGFYPSDISVEWESNGQPENNYKTTPPMLDSDGSFF | |
| | LYSKLTVDKSRWQQGNVFSCSVMHEALHNHYTQKSLSLSPGK | |
| 53 | ASTKGPSVFPLAPCSRSTSESTAALGCLVKDYFPEPVTVSWNSGALTSGVHTFPAVLQSSGLYSLSSVVTV | Human IgG4 |
| | PSSSLGTKTYTCNVDHKPSNTKVDKRVESKYGPPCPSCPAPEFLGGPSVFLFPPKPKDTLMISRTPEVTCV | Fc (Uniprot |
| | VVDVSQEDPEVQFNWYVDGVEVHNAKTKPREEQFNSTYRVVSVLTVLHQDWLNGKEYKCKVSNKGLPSSIE | P01861) |
| | KTISKAKGQPREPQVYTLPPSQEEMTKNQVSLTCLVKGFYPSDIAVEWESNGQPENNYKTTPPVLDSDGSF | |
| | FLYSRLTVDKSRWQEGNVFSCSVMHEALHNHYTQKSLSLSLGK | |
| 54 | MLLLVTSLLLCELPHPAFLLIP | GM-CSF |
| | | signal peptide |
| 55 | MPLLLLLPLLWAGALA | CD33 signal |
| | | peptide |
| 56 | ggatctgcgatcgctccggtgcccgtcagtgggcagagcgcacatcgcccacagtccccgagaagttgggg | EF1alpha |
| | ggaggggtcggcaattgaaccggtgcctagagaaggtggcgcggggtaaactgggaaagtgatgtcgtgta | promoter with |
| | ctggctccgcctttttcccgaggggggggagaaccgtatataagtgcagtagtcgccgtgaacgttctttt | HTLV1 |
| | tcgcaacgggtttgccgccagaacacagctgaagcttegaggggctcgcatctctccttcacgegcccgcc | enhancer |
| | gccctacctgaggccgccatccacgccggttgagtcgcgttctgccgcctcccgcctgtggtgcctcctga | |
| | actgcgtccgccgtctaggtaagtttaaagctcaggtcgagaccgggcctttgtccggcgctcccttggag | |
| | cctacctagactcagccggctctccacgctttgcctgaccctgcttgctcaactctacgtctttgtttcgt | |
| | tttctgttctgcgccgttacagatccaagctgtgaccggcgcctac | |
| 57 | aatcaacctctggattacaaaatttgtgaaagattgactggtattcttaactatgttgctccttttacgct | Woodchuck |
| | atgtggatacgctgctttaatgcctttgtatcatgctattgcttcccgtatggctttcattttctcctcct | Hepatitis Virus |
| | tgtataaatcctggttgctgtctctttatgaggagttgtggcccgttgtcaggcaacgtggcgtggtgtgc | (WHP) |
| | actgtgtttgctgacgcaacccccactggttggggcattgccaccacctgtcagctcctttccgggacttt | Posttranscrip- |
| | cgctttccccctccctattgccacggcggaactcatcgccgcctgccttgcccgctgctggacaggggctc | tional |
| | ggctgttgggcactgacaattccgtggtgttgtcggggaaatcatcgtcctttccttggctgctcgcctgt | Regulatory |
| | gttgccacctggattctgcgcgggacgtccttctgctacgtcccttcggccctcaatccagcggaccttcc | Element (WPRE) |
| | ttcccgcggcctgctgccggctctgeggcctcttccgcgtcttcgccttcgccctcagacgagtcggatct | |
| | ccctttgggcegcctccccgc | |
| 58 | VKQTLNFDLLKLAGDVESNPGP | F2A peptide |
| 59 | GSGVKQTLNFDLLKLAGDVESNPGP | F2A peptide |
| 60 | QCTNYALLKLAGDVESNPGP | E2A peptide |
| 61 | GSGQCTNYALLKLAGDVESNPGP | E2A peptide |
| 62 | LEGGGEGRGSLLTCGDVEENPGPR | T2A peptide |
| 63 | EGRGSLLTCGDVEENPGP | T2A peptide |
| 64 | GSGEGRGSLLTCGDVEENPGP | T2A peptide |
| 65 | GSGATNFSLLKQAGDVEENPGP | P2A peptide |
| 66 | ATNFSLLKQAGDVEENPGP | P2A peptide |
| 67 | DIQMTQTTSSLSASLGDRVTISCRASQDISKYLNWYQQKPDGTVKLLIYHTSRLHSGVPSRFSGSGSGTDY | CD19 scFv |
| | SLTISNLEQEDIATYFCQQGNTLPYTFGGGTKLEITGSTSGSGKPGSGEGSTKGEVKLQESGPGLVAPSQS | |
| | LSVTCTVSGVSLPDYGVSWIRQPPRKGLEWLGVIWGSETTYYNSALKSRLTIIKDNSKSQVFLKMNSLQTD | |
| | DTAIYYCAKHYYYGGSYAMDYWGQGTSVTVSS | |
| 68 | KASQNVGTNVA | CDR L1 |
| 69 | SATYRNS | CDR L2 |
| 70 | QQYNRYPYT | CDR L3 |
| 71 | SYWMN | CDR H1 |
| 72 | QIYPGDGDTNYNGKFKG | CDR H2 |
| 73 | KTISSVVDFYFDY | CDR H3 |
| 74 | EVKLQQSGAELVRPGSSVKISCKASGYAFSSYWMNWVKQRPGQGLEWIGQIYPGDGDTNYNGKFKGQATLT | V_{H} |
| | ADKSSSTAYMQLSGLTSEDSAVYFCARKTISSVVDFYFDYWGQGTTVTVSS | |
| 75 | DIELTQSPKFMSTSVGDRVSVTCKASQNVGTNVAWYQQKPGQSPKPLIYSATYRNSGVPDRFTGSGSGTDF | V_{L} |
| | TLTITNVQSKDLADYFCQQYNRYPYTSGGGTKLEIKR | |
| 76 | ctctatcaatgagagagcaatctcctggtaatgtgatagatttcccaacttaatgccaacataccataaac | TRAC 5′ |
| | ctcccattctgctaatgcccagcctaagttggggagaccactccagattccaagatgtacagtttgctttg | homology arm |
| | ctgggcctttttcccatgcctgcctttactctgccagagttatattgctggggttttgaagaagatcctat | |
| | taaataaaagaataagcagtattattaagtagccctgcatttcaggtttccttgagtggcaggccaggcct | |
| | ggccgtgaacgttcactgaaatcatggcctcttggccaagattgatagcttgtgcctgtccctgagtccca | |
| | gtccatcacgagcagctggtttctaagatgctatttcccgtataaagcatgagaccgtgacttgccagccc | |
| | cacagagccccgcccttgtccatcactggcatctggactccagcctgggttggggcaaagagggaaatgag | |
| | atcatgtcctaaccctgatcctcttgtcccacagatatccagaaccctgaccctgccgtgtaccagctgag | |
| | agactctaaatccagtgacaagtctgtctgcctattcaccgat | |
| 77 | tttgattctcaaacaaatgtgtcacaaagtaaggattctgatgtgtatatcacagacaaaactgtgctaga | TRAC 3′ |
| | catgaggtctatggacttcaagagcaacagtgctgtggcctggagcaacaaatctgactttgcatgtgcaa | homology arm |
| | acgccttcaacaacagcattattccagaagacaccttcttccccagcccaggtaagggcagctttggtgcc | |
| | ttcgcaggctgtttccttgcttcaggaatggccaggttctgcccagagctctggtcaatgatgtctaaaac | |
| | tcctctgattggtggtctcggccttatccattgccaccaaaaccctctttttactaagaaacagtgagcct | |
| | tgttctggcagtccagagaatgacacgggaaaaaagcagatgaagagaaggtggcaggagagggcacgtgg | |
| | cccagcctcagtctctccaactgagttcctgcctgcctgcctttgctcagactgtttgccccttactgctc | |
| | ttctaggcctcattctaagccccttctccaagttgcctctccttatttctccctgtctgccaaaaaatctt | |
| | tcccagctcactaagtcagtctcacgcagtcactcattaacccaccaatcactgattgtg | |
| 78 | MLLLVTSLLLCELPHPAFLLIPDIQMTQTTSSLSASLGDRVTISCRASQDISKYLNWYQQKPDGTVKLLIY | HD Allo CD19 |
| | HTSRLHSGVPSRFSGSGSGTDYSLTISNLEQEDIATYFCQQGNTLPYTFGGGTKLEITGSTSGSGKPGSGE | CAR |
| | GSTKGEVKLQESGPGLVAPSQSLSVTCTVSGVSLPDYGVSWIRQPPRKGLEWLGVIWGSETTYYNSALKSR | (amino acid |
| | LTIIKDNSKSQVFLKMNSLQTDDTAIYYCAKHYYYGGSYAMDYWGQGTSVTVSSESKYGPPCPPCPMFWVL | sequence) |
| | VVVGGVLACYSLLVTVAFIIFWVKRGRKKLLYIFKQPFMRPVQTTQEEDGCSCRFPEEEEGGCELRVKFSR | |
| | SADAPAYQQGQNQLYNELNLGRREEYDVLDKRRGRDPEMGGKPRRKNPQEGLYNELQKDKMAEAYSEIGMK | |
| | GERRRGKGHDGLYQGLSTATKDTYDALHMQALPPR | |
| 79 | AGCAATTGCTATGTCCCAGGCACTCTACTAGACACTTCATACAGTTTAGAAAATCAGATGGGTGTAGATCA | B2M 5′ |
| | AGGCAGGAGCAGGAACCAAAAAGAAAGGCATAAACATAAGAAAAAAAATGGAAGGGGTGGAAACAGAGTAC | homology arm |
| | AATAACATGAGTAATTTGATGGGGGCTATTATGAACTGAGAAATGAACTTTGAAAAGTATCTTGGGGCCAA | (nt) |
| | ATCATGTAGACTCTTGAGTGATGTGTTAAGGAATGCTATGAGTGCTGAGAGGGCATCAGAAGTCCTTGAGA | |
| | GCCTCCAGAGAAAGGCTCTTAAAAATGCAGCGCAATCTCCAGTGACAGAAGATACTGCTAGAAATCTGCTA | |
| | GAAAAAAAACAAAAAAGGCATGTATAGAGGAATTATGAGGGAAAGATACCAAGTCACGGTTTATTCTTCAA | |
| | AATGGAGGTGGCTTGTTGGGAAGGTGGAAGCTCATTTGGCCAGAGTGGAAATGGAATTGGGAGAAATCGAT | |
| | GACCAAATGTAAACACTTGGTGCCTGATATAGCTTGACACCAAGTTAGCCCCAAGTGAAATACCCTGGCAA | |
| | TATTAATGTGTCTTTTCCCGATATTCCTCAGGTACTCCAAAGATTCAGGTTTACTCACGTCATCCAGCAGA | |
| | GAATGGAAAGTCAAATTTCCTGAATTGCTATGTGTCTGGGTTTCATCCATCCGACATTGAAGTTGACTTAC | |
| | TGAAGAATGGAGAGAGAATTGAAAAAGTGGAGCATTCAGACTTGTCTTTCAGCAAGGACTGGTCTTTCTAT | |
| | CTCTTGTACTACACTGAA | |
| 80 | ACTGAAAAAGATGAGTATGCCTGCCGTGTGAACCATGTGACTTTGTCACAGCCCAAGATAGTTAAGTGGGG | B2M 3′ |
| | TAAGTCTTACATTCTTTTGTAAGCTGCTGAAAGTTGTGTATGAGTAGTCATATCATAAAGCTGCTTTGATA | homology arm |
| | TAAAAAAGGTCTATGGCCATACTACCCTGAATGAGTCCCATCCCATCTGATATAAACAATCTGCATATTGG | (nt) |
| | GATTGTCAGGGAATGTTCTTAAAGATCAGATTAGTGGCACCTGCTGAGATACTGATGCACAGCATGGTTTC | |
| | TGAACCAGTAGTTTCCCTGCAGTTGAGCAGGGAGCAGCAGCAGCACTTGCACAAATACATATACACTCTTA | |
| | ACACTTCTTACCTACTGGCTTCCTCTAGCTTTTGTGGCAGCTTCAGGTATATTTAGCACTGAACGAACATC | |
| | TCAAGAAGGTATAGGCCTTTGTTTGTAAGTCCTGCTGTCCTAGCATCCTATAATCCTGGACTTCTCCAGTA | |
| | CTTTCTGGCTGGATTGGTATCTGAGGCTAGTAGGAAGGGCTTGTTCCTGCTGGGTAGCTCTAAACAATGTA | |
| | TTCATGGGTAGGAACAGCAGCCTATTCTGCCAGCCTTATTTCTAACCATTTTAGACATTTGTTAGTACATG | |
| | GTATTTTAAAAGTAAAACTTAATGTCTTCCTTTTTTTTCTCCACTGTCTTTTTCATAGATCGAGACATGTA | |
| | AGCAGCATCATGGAGGTAAGTTTTTGACCTTGAGAAAATGTTTTTGTTTCACTGTCCTGAGGACTATTTAT | |
| | AGACAGCTCTAACATGATA | |
| 81 | MSRSVALAVLALLSLSGLEAVMAPRTLVLGGGGSGGGGSGGGGSIQRTPKIQVYSRHPAENGKSNFLNCYV | HLA-E single |
| | SGFHPSDIEVDLLKNGERIEKVEHSDLSFSKDWSFYLLYYTEFTPTEKDEYACRVNHVTLSQPKIVKWDRD | chain |
| | MGGGGSGGGGSGGGGSGGGGSGSHSLKYFHTSVSRPGRGEPRFISVGYVDDTQFVRFDNDAASPRMVPRAP | fusion(amino |
| | WMEQEGSEYWDRETRSARDTAQIFRVNLRTLRGYYNQSEAGSHTLQWMHGCELGPDGRFLRGYEQFAYDGK | acid sequence) |
| | DYLTLNEDLRSWTAVDTAAQISEQKSNDASEAEHQRAYLEDTCVEWLHKYLEKGKETLLHLEPPKTHVTHH | |
| | PISDHEATLRCWALGFYPAEITLTWQQDGEGHTQDTELVETRPAGDGTFQKWAAVVVPSGEEQRYTCHVQH | |
| | EGLPEPVTLRWKPASQPTIPIVGIIAGLVLLGSVVSGAVVAAVIWRKKSSGGKGGSYYKAEWSDSAQGSES | |
| | HSL | |
| 82 | mG*mA*mG*rArArUrCrArArArArUrCrGrGrUrGrArArUrGrUrUrUrUrArGrArGrCrUrArGrA | TRAC gRNA-A |
| | rArArUrArGrCrArArGrUrUrArArArArUrArArGrGrCrUrArGrUrCrCrGrUrUrArUrCrArAr | m = 2′O-methyl |
| | CrUrUrGrArArArArArGrUrGrGrCrArCrCrGrArGrUrCrGrGrUrGrCrU*mU*mU*mU | RNA base; |
| | | * = phosphoro- |
| | | tiate; |
| | | r = ribonucleo- |
| | | tide |
| 83 | ATGTGTTTTTGTCAAAAGACCTTTTrUrArArUrUrUrCrUrArCrUrCrUrUrGrUrArGrArUrArGrU | B2M gRNA-B |
| | rGrGrGrGrGrUrGrArArUrUrCrArGrUrGrUrA | r = ribonucleo- |
| | | tide |
| 84 | gagaatcaaaatcggtgaat | TRAC target |
| | | site |
| 85 | agtgggggtgaattcagtgta | B2M target site |
| 86 | atgtcaagatctgtcgctctggctgtcctggccctgctgtctctgtctggacttgaagcagtgatggcccc | HLA-E single |
| | tagaacactggtgcttggaggcggaggatctggcggaggtggaagtggcggaggcggatctatccaacgga | chain fusion |
| | cacctaagatccaagtgtacagcagacaccccgccgagaacggcaagagcaacttcctgaactgctacgtg | (DNA) |
| | tccggcttccacccctctgacattgaggtggacctgctgaagaacggcgagcggatcgagaaggtggaaca | |
| | cagcgatctgagcttctccaaggattggtccttctacctgctctactacaccgagttcactcctaccgaga | |
| | aggacgagtacgcatgtagagtgaaccacgtgacactgagccaacctaagatcgtgaaatgggacagagat | |
| | atgggaggcggcggtagtggcggcggaggaagcggaggcggaggttcaggtggtggtggatctggaagcca | |
| | cagcctgaagtactttcacacctccgtgtccagacctggcagaggcgagcctagattcatcagcgtgggct | |
| | acgtggacgacacccagttcgtcagattcgacaacgacgccgcctctcctcggatggttcctagagcaccc | |
| | tggatggaacaagagggcagcgagtactgggatcgcgagacaagaagcgccagagacacagcccagatctt | |
| | ccgcgtgaacctgagaaccctgcggggctactacaatcagtctgaggccggctctcacaccctgcagtgga | |
| | tgcatggatgtgaactgggccccgacggacggttcctgagaggctatgagcagttcgcctacgacggcaag | |
| | gactacctgacactgaacgaggacctgagaagctggaccgccgtggatacagccgctcagatcagcgagca | |
| | gaagtctaacgacgccagcgaggccgaacaccagagagcctatctggaagatacctgcgtggaatggctgc | |
| | acaagtacctggaaaagggcaaagagacactgctgcacctggaaccacctaagacacatgtgacccaccat | |
| | cctatcagcgaccacgaggccacactgagatgttgggccctgggcttttaccctgccgagatcacactgac | |
| | atggcagcaggatggcgagggccacacacaggatacagagctggtggaaacaagacctgccggcgacggca | |
| | ccttccagaaatgggctgctgtggtggttcccagcggcgaggaacagagatacacctgtcacgtgcagcac | |
| | gagggactgcctgaacctgtgactctgagatggaagcctgccagccagccaacaatccccatcgtgggaat | |
| | cattgccggcctggtgctgctgggatctgtggtttctggtgcagtggtggccgccgtgatttggagaaaga | |
| | agtcctctggcggcaaaggcggctcctactataaggccgagtggagcgattctgcccagggctctgaaagc | |
| | cactctctg | |
| 87 | gagaaucaaaaucggugaau | TRAC gRNA- |
| | | A spacer |
| | | sequence |
| 88 | guuuuagagcuagaaauagcaaguuaaaauaaggcuaguccguuaucaacuugaaaaaguggcacc | Cas9 gRNA |
| | gagucggugc | scaffold |
| | | sequence |
| 89 | uaauuucuacucuuguagau | Cas12a gRNA |
| | | scaffold |
| | | sequence |
| 90 | atgtgtttttgtcaaaagacctttt | DNA extension |
| 91 | ggatctgcgatcgctccggtgcccgtcagtgggcagagcgcacatcgcccacagtccccgagaagttgggg | EF1a-HTLV1R |
| | ggaggggtcggcaattgaaccggtgcctagagaaggtggcgcggggtaaactgggaaagtgatgtcgtgta | promoter |
| | ctggctccgcctttttcccgaggggggggagaaccgtatataagtgcagtagtcgccgtgaacgttctttt | |
| | tcgcaacgggtttgccgccagaacacagctgaagcttcgaggggctcgcatctctccttcacgegcccgcc | |
| | gccctacctgaggccgccatccacgccggttgagtcgcgttctgccgcctcccgcctgtggtgcctcctga | |
| | actgcgtccgccgtctaggtaagtttaaagctcaggtcgagaccgggcctttgtccggcgctcccttggag | |
| | cctacctagactcagccggctctccacgctttgcctgaccctgcttgctcaactctacgtctttgtttcgt | |
| | tttctgttctgcgccgttacagatccaagctgtgaccggcgcctac | |
| 92 | gagaaucaaaaucggugaauguuuuagagcuagaaauagcaaguuaaaauaaggcuaguccguuaucaacu | TRAC full |
| | ugaaaaaguggcaccgagucggugcuuuu | gRNA |
| 93 | aatataagtggaggcgtcgcgctggcgggcattcctgaagctgacagcattcgggccgagatgtctcgctc | Homo sapiens |
| | cgtggccttagctgtgctcgcgctactctctctttctggcctggaggctatccagcgtactccaaagattc | beta-2- |
| | aggtttactcacgtcatccagcagagaatggaaagtcaaatttcctgaattgctatgtgtctgggtttcat | microglobulin |
| | ccatccgacattgaagttgacttactgaagaatggagagagaattgaaaaagtggagcattcagacttgtc | (B2M), |
| | tttcagcaaggactggtctttctatctcttgtactacactgaattcacccccactgaaaaagatgagtatg | nucleic acid |
| | cctgccgtgtgaaccatgtgactttgtcacagcccaagatagttaagtgggatcgagacatgtaagcagca | NCBI |
| | tcatggaggtttgaagatgccgcatttggattggatgaattccaaattctgcttgcttgctttttaatatt | Reference |
| | gatatgcttatacacttacactttatgcacaaaatgtagggttataataatgttaacatggacatgatctt | Sequence: |
| | ctttataattctactttgagtgctgtctccatgtttgatgtatctgagcaggttgctccacaggtagctct | NM_004048.2 |
| | aggagggctggcaacttagaggtggggagcagagaattctcttatccaacatcaacatcttggtcagattt | |
| | gaactcttcaatctcttgcactcaaagcttgttaagatagttaagcgtgcataagttaacttccaatttac | |
| | atactctgcttagaatttgggggaaaatttagaaatataattgacaggattattggaaatttgttataatg | |
| | aatgaaacattttgtcatataagattcatatttacttcttatacatttgataaagtaaggcatggttgtgg | |
| | ttaatctggtttatttttgttccacaagttaaataaatcataaaacttgatgtgttatctctta | |
| 94 | ctctatcaatgagagagcaatctcctggtaatgtgatagatttcccaacttaatgccaacataccataaac | HD Allo CD19 |
| | ctcccattctgctaatgcccagcctaagttggggagaccactccagattccaagatgtacagtttgctttg | CAR TRAC KI |
| | ctgggcctttttcccatgcctgcctttactctgccagagttatattgctggggttttgaagaagatcctat | template (DNA |
| | taaataaaagaataagcagtattattaagtagccctgcatttcaggtttccttgagtggcaggccaggcct | sequence) |
| | ggccgtgaacgttcactgaaatcatggcctcttggccaagattgatagcttgtgcctgtccctgagtccca | |
| | gtccatcacgagcagctggtttctaagatgctatttcccgtataaagcatgagaccgtgacttgccagccc | |
| | cacagagccccgcccttgtccatcactggcatctggactccagcctgggttggggcaaagagggaaatgag | |
| | atcatgtcctaaccctgatcctcttgtcccacagatatccagaaccctgaccctgccgtgtaccagctgag | |
| | agactctaaatccagtgacaagtctgtctgcctattcaccgatggatccggatctgcgatcgctccggtgc | |
| | ccgtcagtgggcagagcgcacatcgcccacagtccccgagaagttggggggaggggtcggcaattgaaccg | |
| | gtgcctagagaaggtggcgcggggtaaactgggaaagtgatgtcgtgtactggctccgcctttttcccgag | |
| | gggggggagaaccgtatataagtgcagtagtcgccgtgaacgttctttttcgcaacgggtttgccgccaga | |
| | acacagctgaagcttcgaggggctcgcatctctccttcacgcgcccgccgccctacctgaggccgccatcc | |
| | acgccggttgagtcgcgttctgccgcctcccgcctgtggtgcctcctgaactgcgtccgccgtctaggtaa | |
| | gtttaaagctcaggtcgagaccgggcctttgtccggcgctcccttggagcctacctagactcagccggctc | |
| | tccacgctttgcctgaccctgcttgctcaactctacgtctttgtttcgttttctgttctgcgccgttacag | |
| | atccaagctgtgaccggcgcctacggctagcgccgccaccatgctgctgctggtgaccagcctgctgctgt | |
| | gcgagctgccccaccccgcctttctgctgatccccgacatccagatgacccagaccacctccagcctgagc | |
| | gccagcctgggcgaccgggtgaccatcagctgccgggccagccaggacatcagcaagtacctgaactggta | |
| | tcagcagaagcccgacggcaccgtcaagctgctgatctaccacaccagccggctgcacagcggcgtgccca | |
| | gccggtttagcggcagcggctccggcaccgactacagcctgaccatctccaacctggaacaggaagatatc | |
| | gccacctacttttgccagcagggcaacacactgccctacacctttggcggcggaacaaagctggaaatcac | |
| | cggcagcacctccggcagcggcaagcctggcagcggcgagggcagcaccaagggcgaggtgaagctgcagg | |
| | aaagcggccctggcctggtggcccccagccagagcctgagcgtgacctgcaccgtgagcggcgtgagcctg | |
| | cccgactacggcgtgagctggatccggcagccccccaggaagggcctggaatggctgggcgtgatctgggg | |
| | cagcgagaccacctactacaacagcgccctgaagagccggctgaccatcatcaaggacaacagcaagagcc | |
| | aggtgttcctgaagatgaacagcctgcagaccgacgacaccgccatctactactgcgccaagcactactac | |
| | tacggcggcagctacgccatggactactggggccagggcaccagcgtgaccgtgagcagcgaatctaagta | |
| | cggaccgccctgccccccttgccctatgttctgggtgctggtggtggtcggaggcgtgctggcctgctaca | |
| | gcctgctggtcaccgtggccttcatcatcttttgggtgaaacggggcagaaagaaactcctgtatatattc | |
| | aaacaaccatttatgagaccagtacaaactactcaagaggaagatggctgtagctgccgatttccagaaga | |
| | agaagaaggaggatgtgaactgcgggtgaagttcagcagaagcgccgacgcccctgcctaccagcagggcc | |
| | agaatcagctgtacaacgagctgaacctgggcagaagggaagagtacgacgtcctggataagcggagaggc | |
| | cgggaccctgagatgggggcaagcctcggcggaagaacccccaggaaggcctgtataacgaactgcagaaa | |
| | gacaagatggccgaggcctacagcgagatcggcatgaagggcgagcggaggcggggcaagggccacgacgg | |
| | cctgtatcagggcctgtccaccgccaccaaggatacctacgacgccctgcacatgcaggccctgcccccaa | |
| | ggtgaactagtgtcgactgctttatttgtgaaatttgtgatgctattgctttatttgtaaccattataagc | |
| | tgcaataaacaagttaacaacaacaattgcattcattttatgtttcaggttcagggggaggtgtgggaggt | |
| | tttttaaagggccctttgattctcaaacaaatgtgtcacaaagtaaggattctgatgtgtatatcacagac | |
| | aaaactgtgctagacatgaggtctatggacttcaagagcaacagtgctgtggcctggagcaacaaatctga | |
| | ctttgcatgtgcaaacgccttcaacaacagcattattccagaagacaccttcttccccagcccaggtaagg | |
| | gcagctttggtgccttcgcaggctgtttccttgcttcaggaatggccaggttctgcccagagctctggtca | |
| | atgatgtctaaaactcctctgattggtggtctcggccttatccattgccaccaaaaccctctttttactaa | |
| | gaaacagtgagccttgttctggcagtccagagaatgacacgggaaaaaagcagatgaagagaaggtggcag | |
| | gagagggcacgtggcccagcctcagtctctccaactgagttcctgcctgcctgcctttgctcagactgttt | |
| | gccccttactgctcttctaggcctcattctaagccccttctccaagttgcctctccttatttctccctgtc | |
| | tgccaaaaaatctttcccagctcactaagtcagtctcacgcagtcactcattaacccaccaatcactgatt | |
| | gtg | |
| 95 | VKQTLNFDLLKLAGDVESNPGP | F2A |
| 96 | QCTNYALLKLAGDVESNPGP | E2A |
| 97 | LEGGGEGRGSLLTCGDVEENPGPR | T2A |
| 98 | EGRGSLLTCGDVEENPGP | T2A |
| 99 | GSGATNFSLLKQAGDVEENPGP | P2A |
| 100 | ATNFSLLKQAGDVEENPGP | P2A |
| 101 | (G₄S)₃₋₄ | Linker, |
| | | repeated 3-4 |
| | | times |
| 102 | (G₄S)₂₋₃ | Linker, |
| | | repeated 2-3 |
| | | times |
| 103 | GGGAS(G₄S)₂ | Linker |
| 104 | GCGASGGGGSGGGGS | Linker with |
| | | cysteine trap |
| 105 | agugggggugaauucagugua | B2M gRNA |
| | | spacer |
| | | sequence |
| 106 | atatccagaaccctgaccctgccgtgtaccagctgagagactctaaatccagtgacaagtctgtctgccta | Human TCR |
| | ttcaccgattttgattctcaaacaaatgtgtcacaaagtaaggattctgatgtgtatatcacagacaaaac | alpha constant |
| | tgtgctagacatgaggtctatggacttcaagagcaacagtgctgtggcctggagcaacaaatctgactttg | (TRAC) |
| | catgtgcaaacgccttcaacaacagcattattccagaagacaccttcttccccagcccaggtaagggcagc | NCBI |
| | tttggtgccttcgcaggctgtttccttgcttcaggaatggccaggttctgcccagagctctggtcaatgat | Reference |
| | gtctaaaactcctctgattggtggtctcggccttatccattgccaccaaaaccctctttttactaagaaac | Sequence: |
| | agtgagccttgttctggcagtccagagaatgacacgggaaaaaagcagatgaagagaaggtggcaggagag | NG_001332.3, |
| | ggcacgtggcccagcctcagtctctccaactgagttcctgcctgcctgcctttgctcagactgtttgcccc | TRAC |
| | ttactgctcttctaggcctcattctaagccccttctccaagttgcctctccttatttctccctgtctgcca | |
| | aaaaatctttcccagctcactaagtcagtctcacgcagtcactcattaacccaccaatcactgattgtgcc | |
| | ggcacatgaatgcaccaggtgttgaagtggaggaattaaaaagtcagatgaggggtgtgcccagaggaagc | |
| | accattctagttgggggagcccatctgtcagctgggaaaagtccaaataacttcagattggaatgtgtttt | |
| | aactcagggttgagaaaacagctaccttcaggacaaaagtcagggaagggctctctgaagaaatgctactt | |
| | gaagataccagccctaccaagggcagggagaggaccctatagaggcctgggacaggagctcaatgagaaag | |
| | gagaagagcagcaggcatgagttgaatgaaggaggcagggccgggtcacagggccttctaggccatgagag | |
| | ggtagacagtattctaaggacgccagaaagctgttgatcggcttcaagcaggggagggacacctaatttgc | |
| | ttttcttttttttttttttttttttttttttttttgagatggagttttgctcttgttgcccaggctggagt | |
| | gcaatggtgcatcttggctcactgcaacctccgcctcccaggttcaagtgattctcctgcctcagcctccc | |
| | gagtagctgagattacaggcacccgccaccatgcctggctaattttttgtatttttagtagagacagggtt | |
| | tcactatgttggccaggctggtctcgaactcctgacctcaggtgatccacccgcttcagcctcccaaagtg | |
| | ctgggattacaggcgtgagccaccacacccggcctgcttttcttaaagatcaatctgagtgctgtacggag | |
| | agtgggttgtaagccaagagtagaagcagaaagggagcagttgcagcagagagatgatggaggcctgggca | |
| | gggtggtggcagggaggtaaccaacaccattcaggtttcaaaggtagaaccatgcagggatgagaaagcaa | |
| | agaggggatcaaggaaggcagctggattttggcctgagcagctgagtcaatgatagtgccgtttactaaga | |
| | agaaaccaaggaaaaaatttggggtgcagggatcaaaactttttggaacatatgaaagtacgtgtttatac | |
| | tctttatggcccttgtcactatgtatgcctcgctgcctccattggactctagaatgaagccaggcaagagc | |
| | agggtctatgtgtgatggcacatgtggccagggtcatgcaacatgtactttgtacaaacagtgtatattga | |
| | gtaaatagaaatggtgtccaggagccgaggtatcggtcctgccagggccaggggctctccctagcaggtgc | |
| | tcatatgctgtaagttccctccagatctctccacaaggaggcatggaaaggctgtagttgttcacctgccc | |
| | aagaactaggaggtctggggtgggagagtcagcctgctctggatgctgaaagaatgtctgtttttcctttt | |
| | agaaagttcctgtgatgtcaagctggtcgagaaaagctttgaaacaggtaagacaggggtctagcctgggt | |
| | ttgcacaggattgcggaagtgatgaacccgcaataaccctgcctggatgagggagtgggaagaaattagta | |
| | gatgtgggaatgaatgatgaggaatggaaacagcggttcaagacctgcccagagctgggggggtctctcct | |
| | gaatccctctcaccatctctgactttccattctaagcactttgaggatgagtttctagcttcaatagacca | |
| | aggactctctcctaggcctctgtattcctttcaacagctccactgtcaagagagccagagagagcttctgg | |
| | gtggcccagctgtgaaatttctgagtcccttagggatagccctaaacgaaccagatcatcctgaggacagc | |
| | caagaggttttgccttctttcaagacaagcaacagtactcacataggctgtgggcaatggtcctgtctctc | |
| | aagaatcccctgccactcctcacacccaccctgggcccatattcatttccatttgagttgttcttattgag | |
| | tcatccttcctgtggtagcggaactcactaaggggcccatctggacccgaggtattgtgatgataaattct | |
| | gagcacctaccccatccccagaagggctcagaaataaaataagagccaagtctagtcggtgtttcctgtct | |
| | tgaaacacaatactgttggccctggaagaatgcacagaatctgtttgtaaggggatatgcacagaagctgc | |
| | aagggacaggaggtgcaggagctgcaggcctcccccacccagcctgctctgccttggggaaaaccgtgggt | |
| | gtgtcctgcaggccatgcaggcctgggacatgcaagcccataaccgctgtggcctcttggttttacagata | |
| | cgaacctaaactttcaaaacctgtcagtgattgggttccgaatcctcctcctgaaagtggccgggtttaat | |
| | ctgctcatgacgctgcggctgtggtccagctgaggtgaggggccttgaagctgggagtggggtttagggac | |
| | gcgggtctctgggtgcatcctaagctctgagagcaaacctccctgcagggtcttgcttttaagtccaaagc | |
| | ctgagcccaccaaactctcctacttcttcctgttacaaattcctcttgtgcaataataatggcctgaaacg | |
| | ctgtaaaatatcctcatttcagccgcctcagttgcacttctcccctatgaggtaggaagaacagttgttta | |
| | gaaacgaagaaactgaggccccacagctaatgagtggaggaagagagacacttgtgtacaccacatgcctt | |
| | gtgttgtacttctctcaccgtgtaacctcctcatgtcctctctccccagtacggctctcttagctcagtag | |
| | aaagaagacattacactcatattacaccccaatcctggctagagtctccgcaccctcctcccccagggtcc | |
| | ccagtcgtcttgctgacaactgcatcctgttccatcaccatcaaaaaaaaactccaggctgggtgcggggg | |
| | ctcacacctgtaatcccagcactttgggaggcagaggcaggaggagcacaggagctggagaccagcctggg | |
| | caacacagggagaccccgcctctacaaaaagtgaaaaaattaaccaggtgtggtgctgcacacctgtagtc | |
| | ccagctacttaagaggctgagatgggaggatcgcttgagccctggaatgttgaggctacaatgagctgtga | |
| | ttgcgtcactgcactccagcctggaagacaaagcaagatcctgtctcaaataataaaaaaaataagaactc | |
| | cagggtacatttgctcctagaactctaccacatagccccaaacagagccatcaccatcacatccctaacag | |
| | tcctgggtcttcctcagtgtccagcctgacttctgttcttcctcattccagatctgcaagattgtaagaca | |
| | gcctgtgctccctcgctccttcctctgcattgcccctcttctccctctccaaacagagggaactctcctac | |
| | ccccaaggaggtgaaagctgctaccacctctgtgcccccccggcaatgccaccaactggatcctacccgaa | |
| | tttatgattaagattgctgaagagctgccaaacactgctgccaccccctctgttcccttattgctgcttgt | |
| | cactgcctgacattcacggcagaggcaaggctgctgcagcctcccctggctgtgcacattccctcctgctc | |
| | cccagagactgcctccgccatcccacagatgatggatcttcagtgggttctcttgggctctaggtcctgca | |
| | gaatgttgtgaggggtttatttttttttaatagtgttcataaagaaatacatagtattcttcttctcaaga | |
| | cgtggggggaaattatctcattatcgaggccctgctatgctgtgtatctgggcgtgttgtatgtcctgctg | |
| | ccgatgccttc | |
| 107 | VMAPRTLVL | HLA-E fusion |
| | | protein peptide |
| 108 | VMAPRTLLL | HLA-E fusion |
| | | protein peptide |
| 109 | VMAPRTVL | HLA-E fusion |
| | | protein peptide |
| 110 | VMAPRTLFL | HLA-E fusion |
| | | protein peptide |
| 111 | VMAPRTLIL | HLA-E fusion |
| | | protein peptide |
| 112 | (G4S)3 | Linker, |
| | | repeated 3 |
| | | times |
| 113 | ESKYGPPCPPCP | Short Spacer |
| | | (IgG4 hinge) |
| | | (aa) |
| 114 | gaatctaagtacggaccgccctgccccccttgccct | spacer |
| | | (IgG4hinge) |
| | | (nt) |
| | | homo sapiens |
| 115 | ESKYGPPCPPCPGQPREPQVYTLPPSQEEMTKNQVSLTCLVKGFYPSDIAVEWESNGQPENNYKTTPPVLD | Hinge-CH3 |
| | SDGSFFLYSRLTVDKSRWQEGNVFSCSVMHEALHNHYTQKSLSLSLGK | spacer |
| | | Homo sapiens |
| 116 | RWPESPKAQASSVPTAQPQAEGSLAKATTAPATTRNTGRGGEEKKKEKEKEEQEERETKTPECPSHTQPLG | IgD-hinge-Fc |
| | VYLLTPAVQDLWLRDKATFTCFVVGSDLKDAHLTWEVAGKVPTGGVEEGLLERHSNGSQSQHSRLTLPRSL | Homo sapiens |
| | WNAGTSVTCTLNHPSLPPQRLMALREPAAQAPVKLSLNLLASSDPPEAASWLLCEVSGFSPPNILLMWLED | |
| | QREVNTSGFAPARPPPQPGSTTFWAWSVLRVPAPPSPQPATYTCVVSHEDSRTLLNASRSLEVSYVTDH | |
| 117 | EPKSCDKTHTCPPCP | Hinge |
| 118 | ERKCCVECPPCP | Hinge |
| 119 | ELKTPLGDTHTCPRCPEPKSCDTPPPCPRCPEPKSCDTPPPCPRCPEPKSCDTPPPCPRCP | Hinge |
| 120 | ESKYGPPCPSCP | Hinge |
| 121 | ESKYGPPCPPCP | Hinge |
| 122 | YGPPCPPCP | Hinge |
| 123 | KYGPPCPPCP | Hinge |
| 124 | EVVVKYGPPCPPCP | Hinge |
| 125 | IEVMYPPPYLDNEKSNGTIIHVKGKHLCPSPLFPGPSKPFWVLVVVGGVLACYSLLVTVAFIIFWV | CD28 (amino |
| | | acids 114-179 |
| | | of Accession |
| | | No. P10747) |
| | | Homo sapiens |
| 126 | IYIWAPLAGTCGVLLLSLVITLYC | CD8α |
| | | transmembrane |
| | | domain |
| 127 | TTTPAPRPPTPAPTIASQPLSLRPEACRPAAGGAVHTRGLDFACD | CD8α hinge |
| | | domain |
| 128 | IEVMYPPPYLDNEKSNGTIIHVKGKHLCPSPLFPGPSKP | CD28 hinge |
| | | domain |
| 129 | AAAIEVMYPPPYLDNEKSNGTIIHVKGKHLCPSPLFPGPSKP | CD28 hinge |
| | | domain |
| 130 | RSKRSRLLHSDYMNMTPRRPGPTRKHYQPYAPPRDFAAYRS | CD28 (amino |
| | | acids 180-220 |
| | | of P10747) |
| | | Homo sapiens |
| 131 | RSKRSRGGHSDYMNMTPRRPGPTRKHYQPYAPPRDFAAYRS | CD28 (LL to |
| | | GG) |
| | | Homo sapiens |
| 132 | VMAPRTLVLGGGGSGGGGSGGGGSIQRTPKIQVYSRHPAENGKSNFLNCYVSGFHPSDIEVDLLKNGERIE | mature HLA-E |
| | KVEHSDLSFSKDWSFYLLYYTEFTPTEKDEYACRVNHVTLSQPKIVKWDRDMGGGGSGGGGSGGGGSGGGG | coding |
| | SGSHSLKYFHTSVSRPGRGEPRFISVGYVDDTQFVRFDNDAASPRMVPRAPWMEQEGSEYWDRETRSARDT | sequence |
| | AQIFRVNLRTLRGYYNQSEAGSHTLQWMHGCELGPDGRFLRGYEQFAYDGKDYLTLNEDLRSWTAVDTAAQ | without signal |
| | ISEQKSNDASEAEHQRAYLEDTCVEWLHKYLEKGKETLLHLEPPKTHVTHHPISDHEATLRCWALGFYPAE | peptide |
| | ITLTWQQDGEGHTQDTELVETRPAGDGTFQKWAAVVVPSGEEQRYTCHVQHEGLPEPVTLRWKPASQPTIP | |
| | IVGIIAGLVLLGSVVSGAVVAAVIWRKKSSGGKGGSYYKAEWSDSAQGSESHSL | |
| 133 | GSHSMRYFSAAVSRPGRGEPRFIAMGYVDDTQFVRFDSDSACPRMEPRAPWVEQEGPEYWEEETRNTKAHA | HLA class I |
| | QTDRMNLQTLRGYYNQSEASSHTLQWMIGCDLGSDGRLLRGYEQYAYDGKDYLALNEDLRSWTAADTAAQI | histo- |
| | SKRKCEAANVAEQRRAYLEGTCVEWLHRYLENGKEMLQRADPPKTHVTHHPVFDYEATLRCWALGFYPAEI | compatibility |
| | ILTWQRDGEDQTQDVELVETRPAGDGTFQKWAAVVVPSGEEQRYTCHVQHEGLPEPLMLRWKQSSLPTIPI | antigen, |
| | MGIVAGLVVLAAVVTGAAVAAVLWRKKSSD | alpha chain G |
| | | (NP_002118.1) |
| | | without signal |
| | | peptide |
| 134 | QRTPKIQVYSRHPAENGKSNFLNCYVSGFHPSDIEVDLLKNGERIEKVEHSDLSFSKDWSFYLLYYTEFTP | beta-2- |
| | TEKDEYACRVNHVTLSQPKIVKWDRDM | microglobulin |
| | | mature peptide |
| | | (NCBI |
| | | Reference |
| | | Sequence: |
| | | NP_004039.1) |
| | | (without signal |
| | | peptide) |
| 135 | QLLFNKTKSVEFTFCNDTVVIPCFVTNMEAQNTTEVYVKWKFKGRDIYTFDGALNKSTVPTDFSSAKIEVS | Human CD47 |
| | QLLKGDASLKMDKSDAVSHTGNYTCEVTELTREGETIIELKYRVVSWFSPNENILIVIFPIFAILLFWGQF | (aa) |
| | GIKTLKYRSGGMDEKTIALLVAGLVITVIVIVGAILFVPGEYSLKNATGLGLIVTSTGILILLHYYVFSTA | |
| | IGLTSFVIAILVIQVIAYILAVVGLSLCIAACIPMHGPLLISGLSILALAQLLGLVYMKFVASNQKTIQPP | |
| | RKAVEEPLNAFKESKGMMNDE | |
| 136 | atgctgctgctggtgaccagcctgctgctgtgcgagctgccccaccccgcctttctgctgatccccgacat | HD Allo CD19 |
| | ccagatgacccagaccacctccagcctgagcgccagcctgggcgaccgggtgaccatcagctgccgggcca | CAR (nucleic |
| | gccaggacatcagcaagtacctgaactggtatcagcagaagcccgacggcaccgtcaagctgctgatctac | acid) |
| | cacaccagccggctgcacagcggcgtgcccagccggtttagcggcagcggctccggcaccgactacagcct | |
| | gaccatctccaacctggaacaggaagatatcgccacctacttttgccagcagggcaacacactgccctaca | |
| | cctttggcggcggaacaaagctggaaatcaccggcagcacctccggcagcggcaagcctggcagcggcgag | |
| | ggcagcaccaagggcgaggtgaagctgcaggaaagcggccctggcctggtggcccccagccagagcctgag | |
| | cgtgacctgcaccgtgagcggcgtgagcctgcccgactacggcgtgagctggatccggcagccccccagga | |
| | agggcctggaatggctgggcgtgatctggggcagcgagaccacctactacaacagcgccctgaagagccgg | |
| | ctgaccatcatcaaggacaacagcaagagccaggtgttcctgaagatgaacagcctgcagaccgacgacac | |
| | cgccatctactactgcgccaagcactactactacggcggcagctacgccatggactactggggccagggca | |
| | ccagcgtgaccgtgagcagcgaatctaagtacggaccgccctgccccccttgccctatgttctgggtgctg | |
| | gtggtggtcggaggcgtgctggcctgctacagcctgctggtcaccgtggccttcatcatcttttgggtgaa | |
| | acggggcagaaagaaactcctgtatatattcaaacaaccatttatgagaccagtacaaactactcaagagg | |
| | aagatggctgtagctgccgatttccagaagaagaagaaggaggatgtgaactgcgggtgaagttcagcaga | |
| | agcgccgacgcccctgcctaccagcagggccagaatcagctgtacaacgagctgaacctgggcagaaggga | |
| | agagtacgacgtcctggataagcggagaggccgggaccctgagatgggcggcaagcctcggeggaagaacc | |
| | cccaggaaggcctgtataacgaactgcagaaagacaagatggccgaggcctacagcgagatcggcatgaag | |
| | ggcgagcggaggcggggcaagggccacgacggcctgtatcagggcctgtccaccgccaccaaggataccta | |
| | cgacgccctgcacatgcaggccctgcccccaagg | |
| 137 | agcaattgctatgtcccaggcactctactagacacttcatacagtttagaaaatcagatgggtgtagatca | HLA-E single |
| | aggcaggagcaggaaccaaaaagaaaggcataaacataagaaaaaaaatggaaggggtggaaacagagtac | chain fusion |
| | aataacatgagtaatttgatgggggctattatgaactgagaaatgaactttgaaaagtatcttggggccaa | HDR template |
| | atcatgtagactcttgagtgatgtgttaaggaatgctatgagtgctgagagggcatcagaagtccttgaga | (nucleic acid) |
| | gcctccagagaaaggctcttaaaaatgcagcgcaatctccagtgacagaagatactgctagaaatctgcta | |
| | gaaaaaaaacaaaaaaggcatgtatagaggaattatgagggaaagataccaagtcacggtttattcttcaa | |
| | aatggaggtggcttgttgggaaggtggaagctcatttggccagagtggaaatggaattgggagaaatcgat | |
| | gaccaaatgtaaacacttggtgcctgatatagcttgacaccaagttagccccaagtgaaataccctggcaa | |
| | tattaatgtgtcttttcccgatattcctcaggtactccaaagattcaggtttactcacgtcatccagcaga | |
| | gaatggaaagtcaaatttcctgaattgctatgtgtctgggtttcatccatccgacattgaagttgacttac | |
| | tgaagaatggagagagaattgaaaaagtggagcattcagacttgtctttcagcaaggactggtctttctat | |
| | ctcttgtactacactgaaggatccggcagcggcgagggcagaggcagcctgctgacctgcggcgacgtgga | |
| | ggagaaccccggccccatgtcaagatctgtcgctctggctgtcctggccctgctgtctctgtctggacttg | |
| | aagcagtgatggcccctagaacactggtgcttggaggcggaggatctggcggaggtggaagtggcggaggc | |
| | ggatctatccaacggacacctaagatccaagtgtacagcagacaccccgccgagaacggcaagagcaactt | |
| | cctgaactgctacgtgtccggcttccacccctctgacattgaggtggacctgctgaagaacggcgagcgga | |
| | tcgagaaggtggaacacagcgatctgagcttctccaaggattggtccttctacctgctctactacaccgag | |
| | ttcactcctaccgagaaggacgagtacgcatgtagagtgaaccacgtgacactgagccaacctaagatcgt | |
| | gaaatgggacagagatatgggaggcggcggtagtggcggcggaggaagcggaggcggaggttcaggtggtg | |
| | gtggatctggaagccacagcctgaagtactttcacacctccgtgtccagacctggcagaggcgagcctaga | |
| | ttcatcagcgtgggctacgtggacgacacccagttcgtcagattcgacaacgacgccgcctctcctcggat | |
| | ggttcctagagcaccctggatggaacaagagggcagcgagtactgggatcgcgagacaagaagcgccagag | |
| | acacagcccagatcttccgcgtgaacctgagaaccctgcggggctactacaatcagtctgaggccggctct | |
| | cacaccctgcagtggatgcatggatgtgaactgggccccgacggacggttcctgagaggctatgagcagtt | |
| | cgcctacgacggcaaggactacctgacactgaacgaggacctgagaagctggaccgccgtggatacagccg | |
| | ctcagatcagcgagcagaagtctaacgacgccagcgaggccgaacaccagagagcctatctggaagatacc | |
| | tgcgtggaatggctgcacaagtacctggaaaagggcaaagagacactgctgcacctggaaccacctaagac | |
| | acatgtgacccaccatcctatcagcgaccacgaggccacactgagatgttgggccctgggcttttaccctg | |
| | ccgagatcacactgacatggcagcaggatggcgagggccacacacaggatacagagctggtggaaacaaga | |
| | cctgccggcgacggcaccttccagaaatgggctgctgtggtggttcccagcggcgaggaacagagatacac | |
| | ctgtcacgtgcagcacgagggactgcctgaacctgtgactctgagatggaagcctgccagccagccaacaa | |
| | tccccatcgtgggaatcattgccggcctggtgctgctgggatctgtggtttctggtgcagtggtggccgcc | |
| | gtgatttggagaaagaagtcctctggcggcaaaggggctcctactataaggccgagtggagcgattctgcc | |
| | cagggctctgaaagccactctctgtgaactagtgtcgactgctttatttgtgaaatttgtgatgctattgc | |
| | tttatttgtaaccattataagctgcaataaacaagttaacaacaacaattgcattcattttatgtttcagg | |
| | ttcagggggaggtgtgggaggttttttaaagggcccactgaaaaagatgagtatgcctgccgtgtgaacca | |
| | tgtgactttgtcacagcccaagatagttaagtggggtaagtcttacattcttttgtaagctgctgaaagtt | |
| | gtgtatgagtagtcatatcataaagctgctttgatataaaaaaggtctatggccatactaccctgaatgag | |
| | tcccatcccatctgatataaacaatctgcatattgggattgtcagggaatgttcttaaagatcagattagt | |
| | ggcacctgctgagatactgatgcacagcatggtttctgaaccagtagtttccctgcagttgagcagggagc | |
| | agcagcagcacttgcacaaatacatatacactcttaacacttcttacctactggcttcctctagcttttgt | |
| | ggcagcttcaggtatatttagcactgaacgaacatctcaagaaggtataggcctttgtttgtaagtcctgc | |
| | tgtcctagcatcctataatcctggacttetccagtactttctggctggattggtatctgaggctagtagga | |
| | agggcttgttcctgctgggtagctctaaacaatgtattcatgggtaggaacagcagcctattctgccagcc | |
| | ttatttctaaccattttagacatttgttagtacatggtattttaaaagtaaaacttaatgtcttccttttt | |
| | tttctccactgtctttttcatagatcgagacatgtaagcagcatcatggaggtaagtttttgaccttgaga | |
| | aaatgtttttgtttcactgtcctgaggactatttatagacagctctaacatgata | |
| 138 | DIQMTQTTSSLSASLGDRVTISCRASQDISKYLNWYQQKPDGTVKLLIYHTSRLHSGVPSRFSGSGSGTDY | HD Allo CD19 |
| | SLTISNLEQEDIATYFCQQGNTLPYTFGGGTKLEITGSTSGSGKPGSGEGSTKGEVKLQESGPGLVAPSQS | CAR w/o |
| | LSVTCTVSGVSLPDYGVSWIRQPPRKGLEWLGVIWGSETTYYNSALKSRLTIIKDNSKSQVFLKMNSLQTD | signal peptide |
| | DTAIYYCAKHYYYGGSYAMDYWGQGTSVTVSSESKYGPPCPPCPMFWVLVVVGGVLACYSLLVTVAFIIFW | (amino acid) |
| | VKRGRKKLLYIFKQPFMRPVQTTQEEDGCSCRFPEEEEGGCELRVKFSRSADAPAYQQGQNQLYNELNLGR | |
| | REEYDVLDKRRGRDPEMGGKPRRKNPQEGLYNELQKDKMAEAYSEIGMKGERRRGKGHDGLYQGLSTATKD | |
| | TYDALHMQALPPR | |

What is claimed:

1. A genetically engineered T cell comprising:
(a) a genetic disruption in the endogenous TRAC gene, wherein the genetic disruption in the endogenous TRAC gene is in a target site in exon 1 of the TRAC gene, and wherein the target site in exon 1 of the TRAC gene has the sequence set forth in SEQ ID NO: 84, a contiguous portion thereof of at least 12 nucleotides (nt), or a complementary sequence of the foregoing;
(b) a transgene encoding a CD19 chimeric antigen receptor (CAR) comprising the amino acid sequence set forth in SEQ ID NO: 78, wherein the transgene encoding the CD19 CAR is integrated at the target site in exon 1 of the TRAC gene;
(c) a genetic disruption in the endogenous B-2 microglobulin (B2M) gene; wherein the genetic disruption in the endogenous B2M gene is in a target site in exon 2 of the B2M gene, and wherein the target site in exon 2 of the B2M gene has the sequence set forth in SEQ ID NO: 85, a contiguous portion thereof of at least 12 nucleotides (nt), or a complementary sequence of the foregoing; and
(d) a transgene encoding a single chain HLA-E fusion protein comprising the amino acid sequence set forth in SEQ ID NO: 81, wherein the transgene encoding the single chain HLA-E fusion protein is integrated at the target site in exon 2 of the B2M gene;
wherein the T cell is a primary T cell from a human donor aged 18 to 35 years old and having a body mass index (BMI) less than 30 kg/m².

2. The genetically engineered T cell of claim 1, wherein one or more alleles of the endogenous TRAC gene are disrupted.

3. The genetically engineered T cell of claim 2, wherein the genetically engineered T cell has reduced protein expression of TCR alpha chain encoded from the endogenous TRAC gene.

4. The genetically engineered T cell of claim 1, wherein one or more alleles of the endogenous B2M gene are disrupted.

5. The genetically engineered T cell of claim 4, wherein the genetically engineered T cell has reduced protein expression of B2M encoded from the endogenous B2M gene.

6. The genetically engineered T cell of claim 5, wherein the genetically engineered cell has reduced expression of one or more HLA class I molecules on the cell surface.

7. The genetically engineered T cell of claim 1, wherein the transgene encoding the CD19 CAR comprises the sequence set forth in SEQ ID NO: 136 and the transgene encoding the single chain HLA-E fusion protein comprises the sequence set forth in SEQ ID NO: 86.

8. The genetically engineered T cell of claim 1, wherein the human donor is male or a nulliparous and non-pregnant female.

9. A method of producing a genetically engineered T cell, the method comprising:
(a) introducing into a T cell via electroporation a first CRISPR-Cas system comprising a Cas protein and a guide RNA (gRNA) for inducing a genetic disruption at a target site in exon 1 of an endogenous T cell receptor alpha constant (TRAC) gene; wherein the target site in exon 1 of the endogenous TRAC gene has the sequence set forth in SEQ ID NO: 84, a contiguous portion thereof of at least 12 nucleotides (nt), or a complementary sequence of the foregoing, and the gRNA comprises a spacer sequence that is complementary to the target site;
(b) introducing into the T cell via electroporation a second CRISPR-Cas system comprising a Cas protein and a guide RNA (gRNA) for inducing a genetic disruption at a target site in exon 2 of an endogenous B-2 microglobulin (B2M) gene, wherein the target site in exon 2 of the B2M gene has the sequence set forth in SEQ ID NO: 85, a contiguous portion thereof of at least 12 nucleotides (nt), or a complementary sequence of the foregoing, and the gRNA comprises a spacer sequence that is complementary to the target site;
(c) introducing into the T cell a polynucleotide comprising a transgene encoding a CD19 chimeric antigen receptor (CAR) comprising the amino acid sequence set forth in SEQ ID NO: 78, wherein the introducing is by transduction of a first AAV viral vector comprising the polynucleotide encoding the CD19 CAR; and
(d) introducing into the T cell a polynucleotide comprising a transgene encoding a single chain HLA-E fusion protein comprising the amino acid sequence set forth in SEQ ID NO: 81, wherein the introducing is by transduction of a second AAV viral vector comprising the polynucleotide comprising the transgene encoding the single chain HLA-E fusion protein;
wherein the T cell is a primary T cell from a human donor aged 18 to 35 years old and having a body mass index (BMI) less than 30 kg/m².

10. The method of claim 9, wherein the first CRISPR-Cas system is a ribonucleoprotein (RNP) complex comprising a Cas9 protein and the gRNA.

11. The method of claim 10, wherein the Cas is a S. pyogenes Cas9 (spCas9).

12. The method of claim 9, wherein the spacer sequence of the gRNA complementary to the target site in exon 1 of the endogenous TRAC gene comprises the nucleic acid sequence of SEQ ID NO: 87, or a contiguous portion thereof of at least 12 nt.

13. The method of claim 9, wherein introducing the first CRISPR-Cas system disrupts one or more alleles of the endogenous TRAC gene.

14. The method of claim 13, wherein introducing the first CRISPR-Cas system into the T cell reduces protein expression of TCR alpha chain encoded from the endogenous TRAC gene.

15. The method of claim 9, wherein the second CRISPR-Cas system is a ribonucleoprotein (RNP) complex comprising a Cas12a protein and the gRNA.

16. The method of claim 15, wherein the Cas12a is Francisella novicida Cas12a (FnCas12a), Lachnospiraceae bacterium Cas12a (LbCas12a), Acidaminococcus sp. Cas12a (AsCas12a).

17. The method of claim 9, wherein the spacer sequence of the gRNA complementary to the target site in exon 2 of the endogenous B2M gene comprises the nucleic acid sequence of SEQ ID NO: 105, or a contiguous portion thereof of at least 12 nt.

18. The method of claim 9, wherein introducing the second CRISPR-Cas system disrupts one or more alleles of the endogenous B2M gene.

19. The method of claim 18, wherein introducing the second CRISPR-Cas system reduces protein expression of B2M encoded from the endogenous B2M gene.

20. The method of claim 19, wherein introducing the second CRISPR-Cas system reduces expression of one or more HLA class I molecules on the cell surface.

21. The method of claim 9, wherein the gRNA targeting the endogenous TRAC gene comprises the sequence set forth in SEQ ID NO: 82 or SEQ ID NO: 92.

22. The method of claim 9, wherein the gRNA targeting the endogenous B2M gene comprises the sequence set forth in SEQ ID NO: 83.

23. The method of any claim 9, wherein the transgene encoding a single chain HLA-E fusion protein is integrated via homology directed repair (HDR) at the target site in the B2M gene.

24. The method of claim 9, wherein the polynucleotide comprising the transgene encoding the single chain HLA-E fusion protein further comprises a 5′ homology arm and a 3′ homology arm linked to the transgene, wherein the homology arms comprise a sequence homologous to nucleic acid sequences surrounding the target site sequence in the endogenous B2M gene.

25. The method of claim 24, wherein the 5′ homology arm comprises the sequence set forth in SEQ ID NO: 79 and the 3′ homology arm comprises the sequence set forth in SEQ ID NO: 80.

26. The method of claim 9, wherein the transgene encoding the CD19 CAR is integrated via homology directed repair (HDR) at the target site in the TRAC gene.

27. The method of claim 9, wherein the polynucleotide comprising the transgene encoding the CD19 CAR further comprises a 5′ homology arm and a 3′ homology arm linked to the transgene, wherein the homology arms comprise a sequence homologous to nucleic acid sequences surrounding the target site sequence in the endogenous TRAC gene.

28. The method of claim 27, wherein the 5′ homology arm comprises the sequence set forth in SEQ ID NO: 76 and the 3′ homology arm comprises the sequence set forth in SEQ ID NO: 77.

29. The method of claim 9, wherein a mixture comprising the first AAV vector and the second AAV vector are introduced into the T cell.

30. The method of claim 9, wherein the first viral vector is an AAV6 vector and the second viral vector is an AAV6 vector.

31. The method of claim 9, wherein the polynucleotide comprising the transgene encoding the CD19 CAR comprises the nucleotide sequence set forth in SEQ ID NO: 94.

32. The method of claim 9, wherein the polynucleotide comprising the transgene encoding the single chain HLA-E fusion protein comprises the nucleotide sequence set forth in SEQ ID NO: 137.

33. The method of claim 9, wherein the polynucleotide comprising the transgene encoding the CD19 CAR comprises the nucleotide sequence set forth in SEQ ID NO: 94 and the polynucleotide comprising the transgene encoding the single chain HLA-E fusion protein comprises the nucleotide sequence set forth in SEQ ID NO: 137.

34. The method of claim 9, wherein the transgene encoding the CD19 CAR comprises the nucleotide sequence set forth in SEQ ID NO: 136.

35. The method of claim 9, wherein the transgene encoding the single chain HLA-E fusion protein comprises the nucleotide sequence set forth in SEQ ID NO: 86.

36. The method of claim 9, wherein the first CRISPR-Cas system and second CRISPR-Cas system are electroporated simultaneously.

37. The method of claim 36, wherein after the electroporation, the T cell is transduced with a mixture of the first AAV viral vector and the second AAV viral vector.

38. The method of claim 9, wherein the donor is male or a nulliparous and non-pregnant female.

39. A composition comprising a population of genetically engineered T cells of claim 1.

40. A composition comprising a population of genetically engineered T cells produced by the method of claim 9.

41. The composition of claim 39, wherein:
(i) at least about 70% of the T cells are viable;
(ii) at least about 10% of total alleles have edited TRAC loci;
(iii) at least about 10% of total alleles have edited B2M loci;
(iv) at least about 10% of total alleles have the transgene encoding the CD19 CAR integrated in the TRAC locus;
(v) at least about 10% of total alleles have the transgene encoding the single chain HLA-E fusion protein integrated in the B2M locus;
(vi) at least about 90% of the T cells are CD2+CD5+;
(vii) at least about 50% of the T cells are CD2+CD5+ and express the CD19 CAR;
(viii) the composition comprises at least about 7,500,000 viable CD2+CD5+CD19 CAR+ cells per mL of the composition;
(ix) the composition has less than about 5 EU/mL endotoxin;
(x) the composition has less than about 70,000 TCR+ cells/kg patient weight;
(xi) less than about 5% of total alleles have translocation between TRAC and B2M;
(xii) the composition has no detected bacterial growth;
(xiii) the composition has no detected mycoplasma;
(xiv) the composition has no cytokine-independent growth;
(xv) the composition has no significant unexpected karyotype; and/or
(xvi) the composition is negative for the presence of HIV-1, HIV-2, HTLV-1, HTLV-2, HAV, HBV, HCV, CMV, EBV, HHV6, HHV7, HHV8, and/or B19.

42. The composition of claim 40, wherein:
(i) at least about 70% of the T cells are viable;
(ii) at least about 10% of total alleles have edited TRAC loci;
(iii) at least about 10% of total alleles have edited B2M loci;
(iv) at least about 10% of total alleles have the transgene encoding the CD19 CAR integrated in the TRAC locus;
(v) at least about 10% of total alleles have the transgene encoding the single chain HLA-E fusion protein integrated in the B2M locus;
(vi) at least about 90% of the T cells are CD2+CD5+;
(vii) at least about 50% of the T cells are CD2+CD5+ and express the CD19 CAR;
(viii) the composition comprises at least about 7,500,000 viable CD2+CD5+CD19 CAR+ cells per mL of the composition;
(ix) the composition has less than about 5 EU/mL endotoxin;
(x) the composition has less than about 70,000 TCR+ cells/kg patient weight;
(xi) less than about 5% of total alleles have translocation between TRAC and B2M;
(xii) the composition has no detected bacterial growth;
(xiii) the composition has no detected mycoplasma;
(xiv) the composition has no cytokine-independent growth;
(xv) the composition has no significant unexpected karyotype; and/or
(xvi) the composition is negative for the presence of HIV-1, HIV-2, HTLV-1, HTLV-2, HAV, HBV, HCV, CMV, EBV, HHV6, HHV7, HHV8, and/or B19.

43. A method of treatment comprising administering the genetically engineered T cell of claim 1 to a subject having an autoimmune disease.

44. The method of claim 43, wherein the autoimmune disease is systemic lupus erythematosus (SLE), idiopathic inflammatory myopathies (IIM), multiple sclerosis (MS), systemic sclerosis (SSc), or rheumatoid arthritis (RA).

45. The method of claim 44, wherein about 50×10⁶, about 100×10⁶, about 200×10⁶, about 300×10⁶, or about 450×10⁶ of the genetically engineered T cells are administered to the subject.

46. A method of treatment comprising administering the composition of claim 40 to a subject having an autoimmune disease.

47. The method of claim 46, wherein the autoimmune disease is systemic lupus erythematosus (SLE), idiopathic inflammatory myopathies (IIM), multiple sclerosis (MS), systemic sclerosis (SSc), or rheumatoid arthritis (RA).

48. The method of claim 47, wherein about 50×10⁶, about 100×10⁶, about 200×10⁶, about 300×10⁶, or about 450×10⁶ of the genetically engineered T cells are administered to the subject.

49. A method of treatment comprising administering the composition of claim 41 to a subject having an autoimmune disease.

50. The method of claim 49, wherein the autoimmune disease is systemic lupus erythematosus (SLE), idiopathic inflammatory myopathies (IIM), multiple sclerosis (MS), systemic sclerosis (SSc), or rheumatoid arthritis (RA).

51. The method of claim 50, wherein about 50×10⁶, about 100×10⁶, about 200×10⁶, about 300×10⁶, or about 450×10⁶ of the genetically engineered T cells are administered to the subject.


SEQUENCE LISTING (138 sequences)

SEQ ID NO: 1 - protein, 120 aa, synthetic construct
  REGION 1..120: note = CD19 VH
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 2 - protein, 107 aa, synthetic construct
  REGION 1..107: note = CD19 VL
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 3 - protein, 5 aa, synthetic construct
  REGION 1..5: note = CD19 CDR-H1
DYGVS

SEQ ID NO: 4 - protein, 16 aa, synthetic construct
  REGION 1..16: note = CD19 CDR-H2
VIWGSETTYYNSALKS

SEQ ID NO: 5 - protein, 7 aa, synthetic construct
  REGION 1..7: note = CD19 CDR-H3
YAMDYWG

SEQ ID NO: 6 - protein, 12 aa, synthetic construct
  REGION 1..12: note = CD19 CDR-H3
HYYYGGSYAMDY

SEQ ID NO: 7 - protein, 11 aa, synthetic construct
  REGION 1..11: note = CD19 CDR-L1
RASQDISKYLN

SEQ ID NO: 8 - protein, 7 aa, synthetic construct
  REGION 1..7: note = CD19 CDR-L2
SRLHSGV

SEQ ID NO: 9 - protein, 7 aa, synthetic construct
  REGION 1..7: note = CD19 CDR-L2
HTSRLHS

SEQ ID NO: 10 - protein, 9 aa, synthetic construct
  REGION 1..9: note = CD19 CDR-L3
GNTLPYTFG

SEQ ID NO: 11 - protein, 9 aa, synthetic construct
  REGION 1..9: note = CD19 CDR-L3
QQGNTLPYT

SEQ ID NO: 12 - protein, 13 aa, synthetic construct
  REGION 1..13: note = Short Spacer (IgG4 hinge)
ESKYGPPCPPCPM

SEQ ID NO: 13 - protein, 228 aa, synthetic construct
  REGION 1..228: note = Long spacer (IgG4/IgG2 hinge- IgG2/IgG4 CH2- IgG4 CH3 spacer
ESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - DNA, 683 bp, synthetic construct
  source 1..683: mol_type = other DNA
  misc_feature 1..683: note = IgG4/IgG2 hinge- IgG2/IgG4 CH2- IgG4 CH3 spacer
gaatctaagtacggaccgccctgccctccctgccctgctcctcctgtggctggaccaagcgtgttcctgtttccacctaagcctaaagataccctgatgatttcccgcacacctgaagtgacttgcgtggtcgtggacgtgagccaggaggatccagaagtgcagttcaactggtacgtggacggcgtggaagtccacaatgctaagactaaaccccgagaggaacagtttcagtcaacttaccgggtcgtgagcgtgctgaccgtcctgcatcaggattggctgaacgggaaggagtataagtgcaaagtgtctaataagggactgcctagctccatcgagaaaacaattagtaaggcaaaagggcagcctcgagaaccacaggtgtataccctgccccctagccaggaggaaatgaccaagaaccaggtgtccctgacatgtctggtcaaaggcttctatccaagtacatcgccgtggagtgggaatcaaatgggcagcccgagaacaattacaagaccacaccacccgtgctggactctgatggaagtttctttctgtattccaggctgaccgtggataaatctcgctggcaggagggcaacgtgttctcttgcagtgtcatgcacgaagccctgcacaatcattatacacagaagtcactgagcctgtccctgggcaaa

SEQ ID NO: 15 - protein, 27 aa, synthetic construct
  REGION 1..27: note = CD28 transmembrane domain (aa)
FWVLVVVGGVLACYSLLVTVAFIIFWV

SEQ ID NO: 16 - protein, 42 aa, synthetic construct
  REGION 1..42: note = 4-1BB-derived intracellular costimulatory domain(aa)
KRGRKKLLYIFKQPFMRPVQTTQEEDGCSCRFPEEEEGGCEL

SEQ ID NO: 17 - protein, 112 aa, synthetic construct
  REGION 1..112: note = CD3-zeta derived intracellular signaling domain (aa)
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 15 aa, synthetic construct
  REGION 1..15: note = Linker
GGGGSGGGGSGGGGS

SEQ ID NO: 19 - protein, 18 aa, synthetic construct
  REGION 1..18: note = Linker
GSTSGSGKPGSGEGSTKG

SEQ ID NO: 20 - protein, 5 aa, synthetic construct
  REGION 1..5: note = Linker
GGGGS

SEQ ID NO: 21 - protein, 10 aa, synthetic construct
  REGION 1..10: note = Linker
GGGGSGGGGS

SEQ ID NO: 22 - protein, 20 aa, synthetic construct
  REGION 1..20: note = Linker
GGGGSGGGGSGGGGSGGGGS

SEQ ID NO: 23 - protein, 125 aa, synthetic construct
  REGION 1..125: note = CD19 clone 5 VH; CD19 clone 17 VH
EVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTMVTVSS

SEQ ID NO: 24 - protein, 125 aa, synthetic construct
  REGION 1..125: note = CD19 clone 18 VH; CD19 clone 18B VH; CD19 clone 76 VH
EVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTVVTVSS

SEQ ID NO: 25 - protein, 108 aa, synthetic construct
  REGION 1..108: note = CD19 clone 5 VL
SYELTQDPAVSVALGQTVRITCQGDSLRSYYASWYQQKPGQAPVLVIYDKNNRPSGIPDRFSGSSSGNTASLTITGAQAEDEADYYCNSRDSSGNNWVFGGGTKLTVL

SEQ ID NO: 26 - protein, 110 aa, synthetic construct
  REGION 1..110: note = CD19 clone 17 VL
QSALTQPASVSGSPGQSITIFCTGTSSDVGGYNYVSWYQQLPGTAPKLLIYSNNQRPSGVPDRFSGSKSGTSASLAISGLRSEDEADYYCAAWDDSLSVVFGGGTKLTVL

SEQ ID NO: 27 - protein, 106 aa, synthetic construct
  REGION 1..106: note = CD19 clone 18 VL
QSALTQPRSVSGFPGQSVTISCTGTTSDDVSWYQQHPGKAPQLMLYDVSKRPSGVPHRFSGSRSGRAASLIISGLQTEDEADYFCCSYAGRYNSVLFGGGTKLTVL

SEQ ID NO: 28 - protein, 106 aa, synthetic construct
  REGION 1..106: note = CD19 clone 18B VL
QSALTQPRSVSGFPGQSVTISCTGTTSDDVSWYQQHPGKAPQLMLYDVSKRPSGVPHRFSGSRSGRAASLIISGLQTEDEADYFCSSYAGRYNSVLFGGGTKLTVL

SEQ ID NO: 29 - protein, 109 aa, synthetic construct
  REGION 1..109: note = CD19 clone 76 VL
QSVLTQPPSVSAAPGQEVTISCSGSSSNIGNNYVSWYQQLPGTAPKLLIYDNDKRPSGIPDRFSGSKSGTSATLGITGLQTGDEADYYCGTWDGNLSAVFGGGTKVTVL

SEQ ID NO: 30 - protein, 248 aa, synthetic construct
  REGION 1..248: note = CD19 clone 5 scFv
EVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTMVTVSSGGGGSGGGGSGGGGSSYELTQDPAVSVALGQTVRITCQGDSLRSYYASWYQQKPGQAPVLVIYDKNNRPSGIPDRFSGSSSGNTASLTITGAQAEDEADYYCNSRDSSGNNWVFGGGTKLTVL

SEQ ID NO: 31 - protein, 250 aa, synthetic construct
  REGION 1..250: note = CD19 clone 17 scFv
EVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTMVTVSSGGGGSGGGGSGGGGSQSALTQPASVSGSPGQSITIFCTGTSSDVGGYNYVSWYQQLPGTAPKLLIYSNNQRPSGVPDRFSGSKSGTSASLAISGLRSEDEADYYCAAWDDSLSVVFGGGTKLTVL

SEQ ID NO: 32 - protein, 246 aa, synthetic construct
  REGION 1..246: note = CD19 clone 18 scFv
EVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTVVTVSSGGGGSGGGGSGGGGSQSALTQPRSVSGFPGQSVTISCTGTTSDDVSWYQQHPGKAPQLMLYDVSKRPSGVPHRFSGSRSGRAASLIISGLQTEDEADYFCCSYAGRYNSVLFGGGTKLTVL

SEQ ID NO: 33 - protein, 246 aa, synthetic construct
  REGION 1..246: note = CD19 clone 18B scFv
EVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTVVTVSSGGGGSGGGGSGGGGSQSALTQPRSVSGFPGQSVTISCTGTTSDDVSWYQQHPGKAPQLMLYDVSKRPSGVPHRFSGSRSGRAASLIISGLQTEDEADYFCSSYAGRYNSVLFGGGTKLTVL

SEQ ID NO: 34 - protein, 249 aa, synthetic construct
  REGION 1..249: note = CD19 clone 76 scFv
EVQLVESGGGLVQPGRSLRLSCAASGFTFDDYAMHWVRQAPGKGLEWVSGISWNSGRIGYADSVKGRFTISRDNAKNSLFLQMNSLRAEDTAVYYCARDQGYHYYDSAEHAFDIWGQGTVVTVSSGGGGSGGGGSGGGGSQSVLTQPPSVSAAPGQEVTISCSGSSSNIGNNYVSWYQQLPGTAPKLLIYDNDKRPSGIPDRFSGSKSGTSATLGITGLQTGDEADYYCGTWDGNLSAVFGGGTKVTVL

SEQ ID NO: 35 - protein, 5 aa, synthetic construct
  REGION 1..5: note = CD19 clone 5, 17, 18, 18B, 76 VH CDR1
DYAMH

SEQ ID NO: 36 - protein, 17 aa, synthetic construct
  REGION 1..17: note = CD19 clone 5, 17, 18, 18B, 76 VH CDR2
GISWNSGRIGYADSVKG

SEQ ID NO: 37 - protein, 16 aa, synthetic construct
  REGION 1..16: note = CD19 clone 5, 17, 18, 18B, 76 VH CDR3
DQGYHYYDSAEHAFDI

SEQ ID NO: 38 - protein, 11 aa, synthetic construct
  REGION 1..11: note = CD19 clone 5 VL CDR1
QGDSLRSYYAS

SEQ ID NO: 39 - protein, 7 aa, synthetic construct
  REGION 1..7: note = CD19 clone 5 VL CDR2
DKNNRPS

SEQ ID NO: 40 - protein, 11 aa, synthetic construct
  REGION 1..11: note = CD19 clone 5 VL CDR3
NSRDSSGNNWV

SEQ ID NO: 41 - protein, 14 aa, synthetic construct
  REGION 1..14: note = CD19 clone 17 VL CDR1
TGTSSDVGGYNYVS

SEQ ID NO: 42 - protein, 7 aa, synthetic construct
  REGION 1..7: note = CD19 clone 17 VL CDR2
SNNQRPS

SEQ ID NO: 43 - protein, 10 aa, synthetic construct
  REGION 1..10: note = CD19 clone 17 VL CDR3
AAWDDSLSVV

SEQ ID NO: 44 - protein, 9 aa, synthetic construct
  REGION 1..9: note = CD19 clone 18 VL CDR1; CD19 clone 18B VL CDR1
TGTTSDDVS

SEQ ID NO: 45 - protein, 7 aa, synthetic construct
  REGION 1..7: note = CD19 clone 18 VL CDR2; CD19 clone 18B VL CDR2
DVSKRPS

SEQ ID NO: 46 - protein, 11 aa, synthetic construct
  REGION 1..11: note = CD19 clone 18 VL CDR3
CSYAGRYNSVL

SEQ ID NO: 47 - protein, 11 aa, synthetic construct
  REGION 1..11: note = CD19 clone 18B VL CDR3
SSYAGRYNSVL

SEQ ID NO: 48 - protein, 13 aa, synthetic construct
  REGION 1..13: note = CD19 clone 76 VL CDR1
SGSSSNIGNNYVS

SEQ ID NO: 49 - protein, 7 aa, synthetic construct
  REGION 1..7: note = CD19 clone 76 VL CDR2
DNDKRPS

SEQ ID NO: 50 - protein, 10 aa, synthetic construct
  REGION 1..10: note = CD19 clone 76 VL CDR3
GTWDGNLSAV

SEQ ID NO: 51 - skipped

SEQ ID NO: 52 - protein, 326 aa, synthetic construct
  REGION 1..326: note = Human IgG2 Fc (Uniprot P01859)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 53 - protein, 327 aa, synthetic construct
  REGION 1..327: note = Human IgG4 Fc (Uniprot P01861)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 54 - protein, 22 aa, synthetic construct
  REGION 1..22: note = GM-CSF signal peptide
MLLLVTSLLLCELPHPAFLLIP

SEQ ID NO: 55 - protein, 16 aa, synthetic construct
  REGION 1..16: note = CD33 signal peptide
MPLLLLLPLLWAGALA

SEQ ID NO: 56 - DNA, 544 bp, synthetic construct
  source 1..544: mol_type = other DNA
  misc_feature 1..544: note = EF1alpha promoter with HTLV1 enhancer
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 57 - DNA, 589 bp, synthetic construct
  source 1..589: mol_type = other DNA
  misc_feature 1..589: note = Woodchuck Hepatitis Virus (WHP) Posttranscriptional Regulatory Element (WPRE)
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 58 - protein, 22 aa, synthetic construct
  REGION 1..22: note = F2A peptide
VKQTLNFDLLKLAGDVESNPGP

SEQ ID NO: 59 - protein, 25 aa, synthetic construct
  REGION 1..25: note = F2A peptide
GSGVKQTLNFDLLKLAGDVESNPGP

SEQ ID NO: 60 - protein, 20 aa, synthetic construct
  REGION 1..20: note = E2A peptide
QCTNYALLKLAGDVESNPGP

SEQ ID NO: 61 - protein, 23 aa, synthetic construct
  REGION 1..23: note = E2A peptide
GSGQCTNYALLKLAGDVESNPGP

SEQ ID NO: 62 - protein, 24 aa, synthetic construct
  REGION 1..24: note = T2A peptide
LEGGGEGRGSLLTCGDVEENPGPR

SEQ ID NO: 63 - protein, 18 aa, synthetic construct
  REGION 1..18: note = T2A peptide
EGRGSLLTCGDVEENPGP

SEQ ID NO: 64 - protein, 21 aa, synthetic construct
  REGION 1..21: note = T2A peptide
GSGEGRGSLLTCGDVEENPGP

SEQ ID NO: 65 - protein, 22 aa, synthetic construct
  REGION 1..22: note = P2A peptide
GSGATNFSLLKQAGDVEENPGP

SEQ ID NO: 66 - protein, 19 aa, synthetic construct
  REGION 1..19: note = P2A peptide
ATNFSLLKQAGDVEENPGP

SEQ ID NO: 67 - protein, 245 aa, synthetic construct
  REGION 1..245: note = CD19 scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 68 - protein, 11 aa, synthetic construct
  source 1..11: note = CDR L1
KASQNVGTNVA

SEQ ID NO: 69 - protein, 7 aa, synthetic construct
  REGION 1..7: note = CDR L2
SATYRNS

SEQ ID NO: 70 - protein, 9 aa, synthetic construct
  REGION 1..9: note = CDR L3
QQYNRYPYT

SEQ ID NO: 71 - protein, 5 aa, synthetic construct
  REGION 1..5: note = CDR H1
SYWMN

SEQ ID NO: 72 - protein, 17 aa, synthetic construct
  REGION 1..17: note = CDR H2
QIYPGDGDTNYNGKFKG

SEQ ID NO: 73 - protein, 13 aa, synthetic construct
  REGION 1..13: note = CDR H3
KTISSVVDFYFDY

SEQ ID NO: 74 - protein, 122 aa, synthetic construct
  REGION 1..122: note = VH
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSS

SEQ ID NO: 75 - protein, 108 aa, synthetic construct
  REGION 1..108: note = VL
DIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 76 - DNA, 611 bp, synthetic construct
  source 1..611: mol_type = other DNA
  misc_feature 1..611: note = TRAC 5' homology arm
ctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgat

SEQ ID NO: 77 - DNA, 628 bp, synthetic construct
  source 1..628: mol_type = other DNA
  misc_feature 1..628: note = TRAC 3' homology arm
tttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtg

SEQ ID NO: 78 - protein, 461 aa, synthetic construct
  REGION 1..461: note = HD Allo CD19 CAR (amino acid sequence)
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 79 - DNA, 799 bp, synthetic construct
  source 1..799: mol_type = other DNA
  misc_feature 1..799: note = B2M 5' homology arm (nt)
agcaattgctatgtcccaggcactctactagacacttcatacagtttagaaaatcagatgggtgtagatcaaggcaggagcaggaaccaaaaagaaaggcataaacataagaaaaaaaatggaaggggtggaaacagagtacaataacatgagtaatttgatgggggctattatgaactgagaaatgaactttgaaaagtatcttggggccaaatcatgtagactcttgagtgatgtgttaaggaatgctatgagtgctgagagggcatcagaagtccttgagagcctccagagaaaggctcttaaaaatgcagcgcaatctccagtgacagaagatactgctagaaatctgctagaaaaaaaacaaaaaaggcatgtatagaggaattatgagggaaagataccaagtcacggtttattcttcaaaatggaggtggcttgttgggaaggtggaagctcatttggccagagtggaaatggaattgggagaaatcgatgaccaaatgtaaacacttggtgcctgatatagcttgacaccaagttagccccaagtgaaataccctggcaatattaatgtgtcttttcccgatattcctcaggtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaa

SEQ ID NO: 80 - DNA, 800 bp, synthetic construct
  source 1..800: mol_type = other DNA
  misc_feature 1..800: note = B2M 3' homology arm (nt)
actgaaaaagatgagtatgcctgccgtgtgaaccatgtgactttgtcacagcccaagatagttaagtggggtaagtcttacattcttttgtaagctgctgaaagttgtgtatgagtagtcatatcataaagctgctttgatataaaaaaggtctatggccatactaccctgaatgagtcccatcccatctgatataaacaatctgcatattgggattgtcagggaatgttcttaaagatcagattagtggcacctgctgagatactgatgcacagcatggtttctgaaccagtagtttccctgcagttgagcagggagcagcagcagcacttgcacaaatacatatacactcttaacacttcttacctactggcttcctctagcttttgtggcagcttcaggtatatttagcactgaacgaacatctcaagaaggtataggcctttgtttgtaagtcctgctgtcctagcatcctataatcctggacttctccagtactttctggctggattggtatctgaggctagtaggaagggcttgttcctgctgggtagctctaaacaatgtattcatgggtaggaacagcagcctattctgccagccttatttctaaccattttagacatttgttagtacatggtattttaaaagtaaaacttaatgtcttccttttttttctccactgtctttttcatagatcgagacatgtaagcagcatcatggaggtaagtttttgaccttgagaaaatgtttttgtttcactgtcctgaggactatttatagacagctctaacatgata

SEQ ID NO: 81 - protein, 500 aa, synthetic construct
  REGION 1..500: note = HLA-E single chain fusion (amino acid sequence)
MSRSVALAVLALLSLSGLEAVMAPRTLVLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYYKAEWSDSAQGSESHSL

SEQ ID NO: 82 - RNA, 100 nt, synthetic construct
  source 1..100: mol_type = other RNA
  misc_feature 1..100: note = TRAC gRNA-A
  misc_feature 1..3: note = 2' O-methyl RNA base
  misc_feature 98..100: note = 2' O-methyl RNA base
  misc_feature 1..3: note = 3 prime phosphorothioate linkage
  misc_feature 97..99: note = 3 prime phosphorothioate linkage
gagaatcaaaatcggtgaatgttttagagctagaaatagcaagttaaaataaggctagtccgttatcaacttgaaaaagtggcaccgagtcggtgctttt

SEQ ID NO: 83 - DNA, 66 bp, synthetic construct
  misc_feature 1..25: note = DNA
  misc_feature 26..66: note = RNA
  source 1..66: mol_type = other DNA
  misc_feature 1..66: note = B2M gRNA-B
atgtgtttttgtcaaaagacctttttaatttctactcttgtagatagtgggggtgaattcagtgta

SEQ ID NO: 84 - DNA, 20 bp, synthetic construct
  source 1..20: mol_type = other DNA
  misc_feature 1..20: note = TRAC target site
gagaatcaaaatcggtgaat

SEQ ID NO: 85 - DNA, 21 bp, synthetic construct
  source 1..21: mol_type = other DNA
  misc_feature 1..21: note = B2M target site
agtgggggtgaattcagtgta

SEQ ID NO: 86 - DNA, 1500 bp, synthetic construct
  source 1..1500: mol_type = other DNA
  misc_feature 1..1500: note = HLA-E single chain fusion (DNA)
atgtcaagatctgtcgctctggctgtcctggccctgctgtctctgtctggacttgaagcagtgatggcccctagaacactggtgcttggaggcggaggatctggcggaggtggaagtggcggaggcggatctatccaacggacacctaagatccaagtgtacagcagacaccccgccgagaacggcaagagcaacttcctgaactgctacgtgtccggcttccacccctctgacattgaggtggacctgctgaagaacggcgagcggatcgagaaggtggaacacagcgatctgagcttctccaaggattggtccttctacctgctctactacaccgagttcactcctaccgagaaggacgagtacgcatgtagagtgaaccacgtgacactgagccaacctaagatcgtgaaatgggacagagatatgggaggcggcggtagtggcggcggaggaagcggaggcggaggttcaggtggtggtggatctggaagccacagcctgaagtactttcacacctccgtgtccagacctggcagaggcgagcctagattcatcagcgtgggctacgtggacgacacccagttcgtcagattcgacaacgacgccgcctctcctcggatggttcctagagcaccctggatggaacaagagggcagcgagtactgggatcgcgagacaagaagcgccagagacacagcccagatcttccgcgtgaacctgagaaccctgcggggctactacaatcagtctgaggccggctctcacaccctgcagtggatgcatggatgtgaactgggccccgacggacggttcctgagaggctatgagcagttcgcctacgacggcaaggactacctgacactgaacgaggacctgagaagctggaccgccgtggatacagccgctcagatcagcgagcagaagtctaacgacgccagcgaggccgaacaccagagagcctatctggaagatacctgcgtggaatggctgcacaagtacctggaaaagggcaaagagacactgctgcacctggaaccacctaagacacatgtgacccaccatcctatcagcgaccacgaggccacactgagatgttgggccctgggcttttaccctgccgagatcacactgacatggcagcaggatggcgagggccacacacaggatacagagctggtggaaacaagacctgccggcgacggcaccttccagaaatgggctgctgtggtggttcccagcggcgaggaacagagatacacctgtcacgtgcagcacgagggactgcctgaacctgtgactctgagatggaagcctgccagccagccaacaatccccatcgtgggaatcattgccggcctggtgctgctgggatctgtggtttctggtgcagtggtggccgccgtgatttggagaaagaagtcctctggcggcaaaggcggctcctactataaggccgagtggagcgattctgcccagggctctgaaagccactctctg

SEQ ID NO: 87 - RNA, 20 nt, synthetic construct
  source 1..20: mol_type = other RNA
  misc_feature 1..20: note = TRAC gRNA-A spacer sequence
gagaatcaaaatcggtgaat

SEQ ID NO: 88 - RNA, 76 nt, synthetic construct
  source 1..76: mol_type = other RNA
  misc_feature 1..76: note = Cas9 gRNA scaffold sequence
gttttagagctagaaatagcaagttaaaataaggctagtccgttatcaacttgaaaaagtggcaccgagtcggtgc

SEQ ID NO: 89 - RNA, 20 nt, synthetic construct
  source 1..20: mol_type = other RNA
  misc_feature 1..20: note = Cas12a gRNA scaffold sequence
taatttctactcttgtagat

SEQ ID NO: 90 - DNA, 25 bp, synthetic construct
  source 1..25: mol_type = other DNA
  misc_feature 1..25: note = DNA extension
atgtgtttttgtcaaaagacctttt

SEQ ID NO: 91 - DNA, 544 bp, synthetic construct; identical to SEQ ID NO: 56
  source 1..544: mol_type = other DNA
  misc_feature 1..544: note = EF1a-HTLV1R promoter

SEQ ID NO: 92 - RNA, 100 nt, synthetic construct; identical to SEQ ID NO: 82
  source 1..100: mol_type = other RNA
  misc_feature 1..100: note = TRAC full gRNA

SEQ ID NO: 93 - DNA, 987 bp, Homo sapiens
  source 1..987: mol_type = other DNA
  misc_feature 1..987: note = Homo sapiens beta-2-microglobulin (B2M), nucleic acid NCBI Reference Sequence: NM_004048.2
aatataagtggaggcgtcgcgctggcgggcattcctgaagctgacagcattcgggccgagatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaagcagcatcatggaggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaacttagaggtggggagcagagaattctcttatccaacatcaacatcttggtcagatttgaactcttcaatctcttgcactcaaagcttgttaagatagttaagcgtgcataagttaacttccaatttacatactctgcttagaatttgggggaaaatttagaaatataattgacaggattattggaaatttgttataatgaatgaaacattttgtcatataagattcatatttacttcttatacatttgataaagtaaggcatggttgtggttaatctggtttatttttgttccacaagttaaataaatcataaaacttgatgtgttatctctta

SEQ ID NO: 94 - DNA, 3342 bp, synthetic construct
  source 1..3342: mol_type = other DNA
  misc_feature 1..3342: note = HD Allo CD19 CAR TRAC KI template (DNA sequence)
ctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgatggatccggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggctagcgccgccaccatgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggtgaactagtgtcgactgctttatttgtgaaatttgtgatgctattgctttatttgtaaccattataagctgcaataaacaagttaacaacaacaattgcattcattttatgtttcaggttcagggggaggtgtgggaggttttttaaagggccctttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtg

SEQ ID NO: 95 - protein, 22 aa, synthetic construct
  REGION 1..22: note = F2A
VKQTLNFDLLKLAGDVESNPGP

SEQ ID NO: 96 - protein, 20 aa, synthetic construct
  REGION 1..20: note = E2A
QCTNYALLKLAGDVESNPGP

SEQ ID NO: 97 - protein, 24 aa, synthetic construct
  REGION 1..24: note = T2A
LEGGGEGRGSLLTCGDVEENPGPR

SEQ ID NO: 98 - protein, 18 aa, synthetic construct
  REGION 1..18: note = T2A
EGRGSLLTCGDVEENPGP

SEQ ID NO: 99 - protein, 22 aa, synthetic construct
  REGION 1..22: note = P2A
GSGATNFSLLKQAGDVEENPGP

SEQ ID NO: 100 - protein, 19 aa, synthetic construct
  REGION 1..19: note = P2A
ATNFSLLKQAGDVEENPGP

SEQ ID NO: 101 - protein, 5 aa, synthetic construct
  REPEAT 1..5: note = (G4S)3-4; Linker, repeated 3-4 times
GGGGS

SEQ ID NO: 102 - protein, 5 aa, synthetic construct
  REPEAT 1..5: note = (G4S)2-3; Linker, repeated 2-3 times
GGGGS

SEQ ID NO: 103 - protein, 15 aa, synthetic construct
  REGION 1..15: note = Linker
GGGASGGGGSGGGGS

SEQ ID NO: 104 - protein, 15 aa, synthetic construct
  REGION 1..15: note = Linker with cysteine trap
GCGASGGGGSGGGGS

SEQ ID NO: 105 - skipped

SEQ ID NO: 106 - DNA, 4627 bp, Homo sapiens
  source 1..4627: mol_type = genomic DNA
  misc_feature 1..4627: note = Human TCR alpha constant (TRAC) NCBI Reference Sequence: NG_001332.3, TRAC
atatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacagtattctaaggacgccagaaagctgttgatcggcttcaagcaggggagggacacctaatttgcttttcttttttttttttttttttttttttttttttgagatggagttttgctcttgttgcccaggctggagtgcaatggtgcatcttggctcactgcaacctccgcctcccaggttcaagtgattctcctgcctcagcctcccgagtagctgagattacaggcacccgccaccatgcctggctaattttttgtatttttagtagagacagggtttcactatgttggccaggctggtctcgaactcctgacctcaggtgatccacccgcttcagcctcccaaagtgctgggattacaggcgtgagccaccacacccggcctgcttttcttaaagatcaatctgagtgctgtacggagagtgggttgtaagccaagagtagaagcagaaagggagcagttgcagcagagagatgatggaggcctgggcagggtggtggcagggaggtaaccaacaccattcaggtttcaaaggtagaaccatgcagggatgagaaagcaaagaggggatcaaggaaggcagctggattttggcctgagcagctgagtcaatgatagtgccgtttactaagaagaaaccaaggaaaaaatttggggtgcagggatcaaaactttttggaacatatgaaagtacgtgtttatactctttatggcccttgtcactatgtatgcctcgctgcctccattggactctagaatgaagccaggcaagagcagggtctatgtgtgatggcacatgtggccagggtcatgcaacatgtactttgtacaaacagtgtatattgagtaaatagaaatggtgtccaggagccgaggtatcggtcctgccagggccaggggctctccctagcaggtgctcatatgctgtaagttccctccagatctctccacaaggaggcatggaaaggctgtagttgttcacctgcccaagaactaggaggtctggggtgggagagtcagcctgctctggatgctgaaagaatgtctgtttttccttttagaaagttcctgtgatgtcaagctggtcgagaaaagctttgaaacaggtaagacaggggtctagcctgggtttgcacaggattgcggaagtgatgaacccgcaataaccctgcctggatgagggagtgggaagaaattagtagatgtgggaatgaatgatgaggaatggaaacagcggttcaagacctgcccagagctgggtggggtctctcctgaatccctctcaccatctctgactttccattctaagcactttgaggatgagtttctagcttcaatagaccaaggactctctcctaggcctctgtattcctttcaacagctccactgtcaagagagccagagagagcttctgggtggcccagctgtgaaatttctgagtcccttagggatagccctaaacgaaccagatcatcctgaggacagccaagaggttttgccttctttcaagacaagcaacagtactcacataggctgtgggcaatggtcctgtctctcaagaatcccctgccactcctcacacccaccctgggcccatattcatttccatttgagttgttcttattgagtcatccttcctgtggtagcggaactcactaaggggcccatctggacccgaggtattgtgatgataaattctgagcacctaccccatccccagaagggctcagaaataaaataagagccaagtctagtcggtgtttcctgtcttgaaacacaatactgttggccctggaagaatgcacagaatctgtttgtaaggggatatgcacagaagctgcaagggacaggaggtgcaggagctgcaggcctcccccacccagcctgctctgccttggggaaaaccgtgggtgtgtcctgcaggccatgcaggcctgggacatgcaagcccataaccgctgtggcctcttggttttacagatacgaacctaaactttcaaaacctgtcagtgattgggttccgaatcctcctcctgaaagtggccgggtttaatctgctcatgacgctgcggctgtggtccagctgaggtgaggggccttgaagctgggagtggggtttagggacgcgggtctctgggtgcatcctaagctctgagagcaaacctccctgcagggtcttgcttttaagtccaaagcctgagcccaccaaactctcctacttcttcctgttacaaattcctcttgtgcaataataatggcctgaaacgctgtaaaatatcctcatttcagccgcctcagttgcacttctcccctatgaggtaggaagaacagttgtttagaaacgaagaaactgaggccccacagctaatgagtggaggaagagagacacttgtgtacaccacatgccttgtgttgtacttctctcaccgtgtaacctcctcatgtcctctctccccagtacggctctcttagctcagtagaaagaagacattacactcatattacaccccaatcctggctagagtctccgcaccctcctcccccagggtccccagtcgtcttgctgacaactgcatcctgttccatcaccatcaaaaaaaaactccaggctgggtgcgggggctcacacctgtaatcccagcactttgggaggcagaggcaggaggagcacaggagctggagaccagcctgggcaacacagggagaccccgcctctacaaaaagtgaaaaaattaaccaggtgtggtgctgcacacctgtagtcccagctacttaagaggctgagatgggaggatcgcttgagccctggaatgttgaggctacaatgagctgtgattgcgtcactgcactccagcctggaagacaaagcaagatcctgtctcaaataataaaaaaaataagaactccagggtacatttgctcctagaactctaccacatagccccaaacagagccatcaccatcacatccctaacagtcctgggtcttcctcagtgtccagcctgacttctgttcttcctcattccagatctgcaagattgtaagacagcctgtgctccctcgctccttcctctgcattgcccctcttctccctctccaaacagagggaactctcctacccccaaggaggtgaaagctgctaccacctctgtgcccccccggcaatgccaccaactggatcctacccgaatttatgattaagattgctgaagagctgccaaacactgctgccaccccctctgttcccttattgctgcttgtcactgcctgacattcacggcagaggcaaggctgctgcagcctcccctggctgtgcacattccctcctgctccccagagactgcctccgccatcccacagatgatggatcttcagtgggttctcttgggctctaggtcctgcagaatgttgtgaggggtttatttttttttaatagtgttcataaagaaatacatagtattcttcttctcaagacgtggggggaaattatctcattatcgaggccctgctatgctgtgtatctgggcgtgttgtatgtcctgctgccgatgccttc

SEQ ID NO: 107 - protein, 9 aa, synthetic construct
  REGION 1..9: note = HLA-E fusion protein peptide
VMAPRTLVL

SEQ ID NO: 108 - protein, 9 aa, synthetic construct
  REGION 1..9: note = HLA-E fusion protein peptide
VMAPRTLLL

SEQ ID NO: 109 - protein, 8 aa, synthetic construct
  REGION 1..8: note = HLA-E fusion protein peptide
VMAPRTVL

SEQ ID NO: 110 - protein, 9 aa, synthetic construct
  REGION 1..9: note = HLA-E fusion protein peptide
VMAPRTLFL

SEQ ID NO: 111 - protein, 9 aa, synthetic construct
  REGION 1..9: note = HLA-E fusion protein peptide
VMAPRTLIL

SEQ ID NO: 112 - protein, 15 aa, synthetic construct
  REGION 1..15: note = G4S linker repeated 3 times
GGGGSGGGGSGGGGS

SEQ ID NO: 113 - protein, 12 aa, synthetic construct
  REGION 1..12: note = Short Spacer (IgG4 hinge) (aa)
ESKYGPPCPPCP

SEQ ID NO: 114 - DNA, 36 bp, synthetic construct
  source 1..36: mol_type = other DNA
  misc_feature 1..36: note = spacer (IgG4hinge) (nt) homo sapiens
gaatctaagtacggaccgccctgccccccttgccct

SEQ ID NO: 115 - protein, 119 aa, synthetic construct
  REGION 1..119: note = Hinge-CH3 spacer Homo sapiens
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 116 - protein, 282 aa, synthetic construct
  REGION 1..282: note = IgD-hinge-Fc Homo sapiens
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 117 - protein, 15 aa, synthetic construct
  REGION 1..15: note = Hinge
EPKSCDKTHTCPPCP

SEQ ID NO: 118 - protein, 12 aa, synthetic construct
  REGION 1..12: note = Hinge
ERKCCVECPPCP

SEQ ID NO: 119 - protein, 61 aa, synthetic construct
  REGION 1..61: note = Hinge
ELKTPLGDTHTCPRCPEPKSCDTPPPCPRCPEPKSCDTPPPCPRCPEPKSCDTPPPCPRCP

SEQ ID NO: 120 - protein, 12 aa, synthetic construct
  REGION 1..12: note = Hinge
ESKYGPPCPSCP

SEQ ID NO: 121 - protein, 12 aa, synthetic construct
  REGION 1..12: note = Hinge
ESKYGPPCPPCP

SEQ ID NO: 122 - protein, 9 aa, synthetic construct
  REGION 1..9: note = Hinge
YGPPCPPCP

SEQ ID NO: 123 - protein, 10 aa, synthetic construct
  REGION 1..10: note = Hinge
KYGPPCPPCP

SEQ ID NO: 124 - protein, 14 aa, synthetic construct
  REGION 1..14: note = Hinge
EVVVKYGPPCPPCP

SEQ ID NO: 125 - protein, 66 aa, synthetic construct
  REGION 1..66: note = CD28 (amino acids 114-179 of Accession No. P10747) Homo sapiens
IEVMYPPPYLDNEKSNGTIIHVKGKHLCPSPLFPGPSKPFWVLVVVGGVLACYSLLVTVAFIIFWV

SEQ ID NO: 126 - protein, 24 aa, synthetic construct
  REGION 1..24: note = CD8alpha transmembrane domain
IYIWAPLAGTCGVLLLSLVITLYC

SEQ ID NO: 127 - protein, 45 aa, synthetic construct
  REGION 1..45: note = CD8alpha hinge domain
TTTPAPRPPTPAPTIASQPLSLRPEACRPAAGGAVHTRGLDFACD

SEQ ID NO: 128 - protein, 39 aa, synthetic construct
  REGION 1..39: note = CD28 hinge domain
IEVMYPPPYLDNEKSNGTIIHVKGKHLCPSPLFPGPSKP

SEQ ID NO: 129 - protein, 42 aa, synthetic construct
  REGION 1..42: note = CD28 hinge domain
AAAIEVMYPPPYLDNEKSNGTIIHVKGKHLCPSPLFPGPSKP

SEQ ID NO: 130 - protein, 41 aa, synthetic construct
  REGION 1..41: note = CD28 (amino acids 180-220 of P10747) Homo sapiens
RSKRSRLLHSDYMNMTPRRPGPTRKHYQPYAPPRDFAAYRS

SEQ ID NO: 131 - protein, 41 aa, synthetic construct
  REGION 1..41: note = CD28 (LL to GG) Homo sapiens
RSKRSRGGHSDYMNMTPRRPGPTRKHYQPYAPPRDFAAYRS

SEQ ID NO: 132 - protein, 480 aa, synthetic construct
  REGION 1..337: note = mature HLA-E coding sequence without signal peptide
VMAPRTLVLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYYKAEWSDSAQGSESHSL

SEQ ID NO: 133 - protein, 314 aa, synthetic construct
  REGION 1..314: note = HLA class I histocompatibility antigen, alpha chain G (NP_002118.1) without signal peptide
GSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 134 - protein, 98 aa, synthetic construct
  REGION 1..98: note = beta-2-microglobulin mature peptide (NCBI Reference Sequence: NP_004039.1) (without signal peptide)
QRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 135 - protein, 305 aa, synthetic construct
  REGION 1..305: note = Human CD47 (aa)
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 136 - DNA, 1383 bp, synthetic construct
  source 1..1383: mol_type = other DNA
  misc_feature 1..1383: note = HD Allo CD19 CAR (nucleic acid)
atgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 137 - DNA, 3322 bp, synthetic construct
  source 1..3322: mol_type = other DNA
  misc_feature 1..3322: note = HLA-E single chain fusion HDR template (nucleic acid)
agcaattgctatgtcccaggcactctactagacacttcatacagtttagaaaatcagatgggtgtagatcaaggcaggagcaggaaccaaaaagaaaggcataaacataagaaaaaaaatggaaggggtggaaacagagtacaataacatgagtaatttgatgggggctattatgaactgagaaatgaactttgaaaagtatcttggggccaaatcatgtagactcttgagtgatgtgttaaggaatgctatgagtgctgagagggcatcagaagtccttgagagcctccagagaaaggctcttaaaaatgcagcgcaatctccagtgacagaagatactgctagaaatctgctagaaaaaaaacaaaaaaggcatgtatagaggaattatgagggaaagataccaagtcacggtttattcttcaaaatggaggtggcttgttgggaaggtggaagctcatttggccagagtggaaatggaattgggagaaatcgatgaccaaatgtaaacacttggtgcctgatatagcttgacaccaagttagccccaagtgaaataccctggcaatattaatgtgtcttttcccgatattcctcaggtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaaggatccggcagcggcgagggcagaggcagcctgctgacctgcggcgacgtggaggagaaccccggccccatgtcaagatctgtcgctctggctgtcctggccctgctgtctctgtctggacttgaagcagtgatggcccctagaacactggtgcttggaggcggaggatctggcggaggtggaagtggcggaggcggatctatccaacggacacctaagatccaagtgtacagcagacaccccgccgagaacggcaagagcaacttcctgaactgctacgtgtccggcttccacccctctgacattgaggtggacctgctgaagaacggcgagcggatcgagaaggtggaacacagcgatctgagcttctccaaggattggtccttctacctgctctactacaccgagttcactcctaccgagaaggacgagtacgcatgtagagtgaaccacgtgacactgagccaacctaagatcgtgaaatgggacagagatatgggaggcggcggtagtggcggcggaggaagcggaggcggaggttcaggtggtggtggatctggaagccacagcctgaagtactttcacacctccgtgtccagacctggcagaggcgagcctagattcatcagcgtgggctacgtggacgacacccagttcgtcagattcgacaacgacgccgcctctcctcggatggttcctagagcaccctggatggaacaagagggcagcgagtactgggatcgcgagacaagaagcgccagagacacagcccagatcttccgcgtgaacctgagaaccctgcggggctactacaatcagtctgaggccggctctcacaccctgcagtggatgcatggatgtgaactgggccccgacggacggttcctgagaggctatgagcagttcgcctacgacggcaaggactacctgacactgaacgaggacctgagaagctggaccgccgtggatacagccgctcagatcagcgagcagaagtctaacgacgccagcgaggccgaacaccagagagcctatctggaagatacctgcgtggaatggctgcacaagtacctggaaaagggcaaagagacactgctgcacctggaaccacctaagacacatgtgacccaccatcctatcagcgaccacgaggccacactgagatgttgggccctgggcttttaccctgccgagatcacactgacatggcagcaggatggcgagggccacacacaggatacagagctggtggaaacaagacctgccggcgacggcaccttccagaaatgggctgctgtggtggttcccagcggcgaggaacagagatacacctgtcacgtgcagcacgagggactgcctgaacctgtgactctgagatggaagcctgccagccagccaacaatccccatcgtgggaatcattgccggcctggtgctgctgggatctgtggtttctggtgcagtggtggccgccgtgatttggagaaagaagtcctctggcggcaaaggcggctcctactataaggccgagtggagcgattctgcccagggctctgaaagccactctctgtgaactagtgtcgactgctttatttgtgaaatttgtgatgctattgctttatttgtaaccattataagctgcaataaacaagttaacaacaacaattgcattcattttatgtttcaggttcagggggaggtgtgggaggttttttaaagggcccactgaaaaagatgagtatgcctgccgtgtgaaccatgtgactttgtcacagcccaagatagttaagtggggtaagtcttacattcttttgtaagctgctgaaagttgtgtatgagtagtcatatcataaagctgctttgatataaaaaaggtctatggccatactaccctgaatgagtcccatcccatctgatataaacaatctgcatattgggattgtcagggaatgttcttaaagatcagattagtggcacctgctgagatactgatgcacagcatggtttctgaaccagtagtttccctgcagttgagcagggagcagcagcagcacttgcacaaatacatatacactcttaacacttcttacctactggcttcctctagcttttgtggcagcttcaggtatatttagcactgaacgaacatctcaagaaggtataggcctttgtttgtaagtcctgctgtcctagcatcctataatcctggacttctccagtactttctggctggattggtatctgaggctagtaggaagggcttgttcctgctgggtagctctaaacaatgtattcatgggtaggaacagcagcctattctgccagccttatttctaaccattttagacatttgttagtacatggtattttaaaagtaaaacttaatgtcttccttttttttctccactgtctttttcatagatcgagacatgtaagcagcatcatggaggtaagtttttgaccttgagaaaatgtttttgtttcactgtcctgaggactatttatagacagctctaacatgata

SEQ ID NO: 138 - protein, 439 aa, synthetic construct
  REGION 1..439: note = HD Allo CD19 CAR w/o signal peptide (amino acid)
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR